(19) United States
(12) Patent Application Publication
(10) Pub. No.: US 2026/0146049 A1
(43) Pub. Date: May 28, 2026

(54) SPIROCYCLIC ANNULATED 2-AMINO-3-CYANO THIOPHENES AND DERIVATIVES FOR THE TREATMENT OF CANCER

(71) Applicants: Boehringer Ingelheim International GmbH, Ingelheim am Rhein (DE); Vanderbilt University, Nashville, TN (US)

(72) Inventors: Juergen RAMHARTER, Vienna (AT); Tobias BIBERGER, Vienna (AT); Julian FUCHS, Vienna (AT); Bernhard KLUGER, Korneuburg (AT); Sorin-Claudiu ROSCA, Vienna (AT); Dhruba SARKAR, Bangalore (IN); Philipp SCHMALHORST, Vienna (AT); Heinz STADTMUELLER, Weigelsdorf (AT); Alex WATERSON, Murfreesboro, TN (US)

(21) Appl. No.: 19/401,444

(22) Filed: Nov. 26, 2025

Related U.S. Application Data
(60) Provisional application No. 63/790,120, filed on Apr. 17, 2025.

(30) Foreign Application Priority Data
Nov. 27, 2024 (EP) 24215734.5

(51) Int. Cl. C07D 519/00; A61K 31/52; A61K 31/5386; A61K 31/553; A61P 35/00

(52) U.S. Cl. CPC C07D 519/00; A61K 31/52; A61K 31/5386; A61K 31/553; A61P 35/00

(57) ABSTRACT

The present invention encompasses compounds of formula (1)

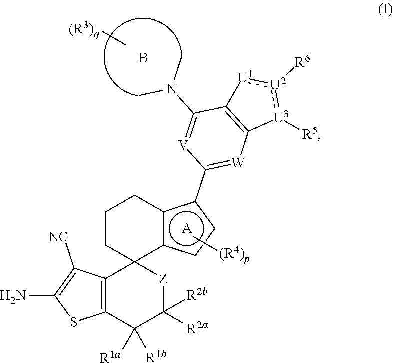

wherein R^{1a}, R^{1b}, R^{2a}, R^{2b}, Z, R³ to R⁶, ring A, ring B, p, q, U¹, U², U³, V and W have the meanings given in the claims and specification, their use as inhibitors of KRAS mutated in position 12, preferrably G12D, pharmaceutical compositions and preparations containing such compounds and their use as medicaments/medical uses, especially as agents for treatment and/or prevention of oncological diseases.

### RELATED APPLICATIONS

The present invention relates to and claims benefit of priority to U.S. Provisional Application No. 63/790,120 filed on Apr. 17, 2025, and European Appl. No. EP24215734.5 filed Nov. 27, 2024, the contents of both are which are incorporated by reference in their entirety herein

### FIELD OF THE INVENTION

The present invention relates to annulated 2-amino-3-cyano thiophenes and derivatives of formula (I)

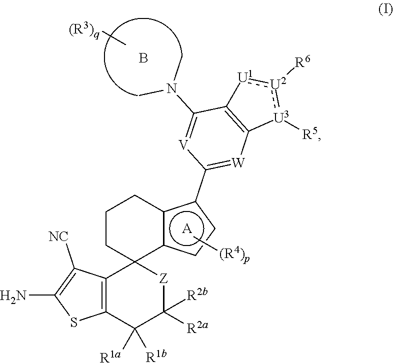

wherein R^{1a}, R^{1b}, R^{2a}, R^{2b}, Z, R³ to R⁶, ring A, ring B, p, q, U¹, U², U³, V and W have the meanings given in the claims and specification, their use as inhibitors of KRAS mutated in position 12, preferably G12D, pharmaceutical compositions and preparations containing such compounds and their use as medicaments/medical uses, especially as agents for treatment and/or prevention of oncological diseases, e.g. cancer.

### BACKGROUND OF THE INVENTION

V-Ki-ras2 Kirsten rat sarcoma viral oncogene homolog (KRAS) is a small GTPase of the Ras family of proteins that exists in cells in either GTP-bound or GDP-bound states (McCormick et al., J. Mol. Med. (Berl)., 2016, 94(3):253-8; Nimnual et al., Sci. STKE., 2002, 2002(145):pe36).

KRAS mutations (e.g. amino acids G12, G13, Q61, A146) are found in a variety of human cancers including lung cancer, colorectal cancer and pancreatic cancer (Cox et al., Nat. Rev. Drug Discov., 2014, 13(11):828-51). Alterations (e.g. mutation, over-expression, gene amplification) in Ras family proteins/Ras genes have also been described as a resistance mechanism against cancer drugs such as the EGFR antibodies cetuximab and panitumumab (Leto et al., J. Mol. Med. (Berl). 2014 July; 92(7):709-22) and the EGFR tyrosine kinase inhibitor osimertinib/AZD9291 (Ortiz-Cuaran et al., Clin. Cancer Res., 2016, 22(19):4837-47; Eberlein et al., Cancer Res., 2015, 7 5(12):2489-500).

Genetic alterations affecting e.g. codon 12 of KRAS substitute the glycine residue naturally occurring at this position for different amino acids such as aspartic acid (the G12D mutation or KRAS G12D), cysteine (the G12C mutation or KRAS G12C), valine (the G12V mutation or KRAS G12V) among others. Similarly, mutations within codons 13, 61 and 146 of KRAS are commonly found in the KRAS gene. Altogether KRAS mutations are detectable in 35% of lung, 45% of colorectal and up to 90% of pancreatic cancers (Herdeis et al., Curr Opin Struct Biol., 2021, 71:136-147). Here especially the KRAS G12D mutation was associated with shorter survival in lung cancer and pancreatic cancer (Ricciuti, B. et al. Ann. Oncol., 2022, 33:1029-1040; Rachakonda, P. S. et al., PLoS One. 2013; 8(4): e60870).

Therefore, binders/inhibitors of mutated KRAS especially G12D are expected to deliver a clear clinical benefit. So far, only the covalent G12C inhibitors sotorasib (AMG510) and adagrasib (MRTX849) have received accelerated approval whereas development of other (K)RAS inhibitors remains a challenge. Due to this high unmet medical need numerous efforts have been reported on the one hand for G12D selective inhibitors (WO2021041671 or WO2023060253) but also KRAS wild type inhibitors (WO2023099624) and on the other hand inhibitors active over a broader range of mutations including the whole RAS family (WO2021091956).

Therefore, the need remains high for safer and efficacious new compounds in the treatment of cancers mediated by KRAS, especially G12D mutation. KRAS inhibitors with a high target selectivity for G12D over RAS and/or KRAS wild type inhibitors are expected to be advantageous. Selective inhibitors may reduce toxicity as high target selectivity may lead to a reduced number of side effects. Additionally desired are beneficial pharmacological properties include but are not limited to oral availability, metabolic stability and permeability.

### SUMMARY OF THE INVENTION

The present invention relates to annulated 2-amino-3-cyano thiophenes and derivatives of formula (I)

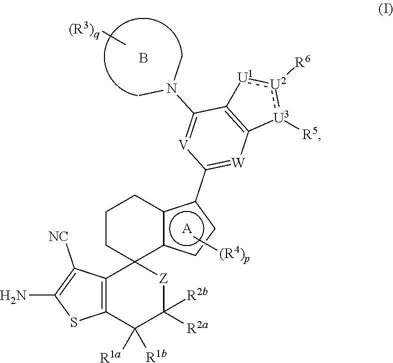

wherein R^{1a}, R^{1b}, R^{2a}, R^{2b}, Z, R³ to R⁶, ring A, ring B, p, q, U¹, U², U³, V and W have the meanings given in the claims and specification, their use as inhibitors of KRAS mutated in position 12, preferably G12D, pharmaceutical compositions and preparations containing such compounds and their use as medicaments/medical uses, especially as agents for treatment and/or prevention of oncological diseases, e.g. cancer.

Surprisingly, the compounds described herein have been found to possess anti-tumour activity, being useful in inhibiting the uncontrolled cellular proliferation which arises from malignant diseases. It is believed that this anti-tumor activity is, inter alia, derived from inhibition of KRAS mutated in position 12, preferably G12D. Advantageously, the compounds are selective for certain KRAS mutants, preferably KRAS G12D. The compounds show at least a 10-fold, preferably a 100-fold, target selectivity for G12D over pan-RAS and/or KRAS wild type inhibitors.

In addition, the compounds of the invention advantageously possess desirable pharmacological properties, including but not limited to low clearance or volume of distribution to provide efficacious treatments.

### DETAILED DESCRIPTION OF THE INVENTION

It has now been found that, surprisingly, compounds of formula (I)

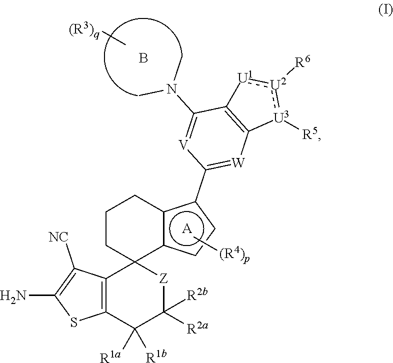

wherein
R^{1a}, R¹¹, R^{2a}, R^{2b}, Z, R³ to R⁶, ring A, ring B, p, q, U¹, U², U³, V and W have the meanings given herein after act as inhibitors of KRAS and are involved in controlling cell proliferation. Thus, the compounds according to the invention may be used for example for the treatment of diseases characterized by excessive or abnormal cell proliferation.

Surprisingly, the compounds described herein have been found to possess anti-tumour activity, being useful in inhibiting the uncontrolled cellular proliferation which arises from malignant diseases. It is believed that this anti-tumor activity is, inter alia, derived from inhibition of KRAS mutated in position 12, preferably G12D. Advantageously, the compounds can be selective for certain KRAS mutants, preferably KRAS G12D. The compounds show at least a 10-fold, preferably a 100-fold, target selectivity for G12D over pan-RAS and/or KRAS wild type inhibitors.

In addition, the compounds of the invention advantageously possess desirable pharmacological properties, including but not limited to low clearance or volume of distribution to provide efficacious treatments.

Thus, in a first aspect, the present invention relates to a compound of formula (I)

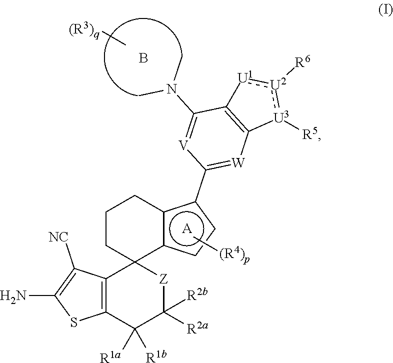

wherein
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl; and/or, optionally, one of R^{1a} or R^{1b} and one of R^{2a} or R^{2b} together with the carbon atoms they are attached to form a cyclopropane ring;
Z is —(CR^{7a}R^{7b})ₙ—;
each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
or R^{7a} and R^{7b} together with the carbon atom they are attached to form a cyclopropane ring;
n is selected from the group consisting of 0, 1 and 2;
W is nitrogen (—N═) or —CH═; V is nitrogen (—N═) or —CH═;
ring A is a ring selected from the group consisting of pyrrole, furan, thiophene, imidazole, pyrazole, oxazole, isoxazole, thiazole, isothiazole and triazole;
each R⁴, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
p is selected from the group consisting of 0, 1, 2 and 3;
U¹ is nitrogen (—N═) or sulphur (—S—);
U² is nitrogen (—N═) or —CR⁶═;
U³ is nitrogen (—NR⁵—) or —CR⁵═;
ring B is a 4-11 membered heterocycle;
each R³, if present, is independently selected from the group consisting of R⁸ and R⁹;
   each R⁸ is independently selected from the group consisting of —OR⁹, —NR⁹R⁹, halogen, and —CN;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl; wherein the C₁₋₆alkyl, C₃₋₁₀cycloalkyl, and 3-11 membered heterocyclyl are all optionally substituted with one or more identical or different R¹²
   each R¹² is independently selected from the group consisting of halogen, C₁₋₆alkyl, C₁₋₆alkoxy, C₁₋₆haloalkyl;
q is selected from the group consisting of 0, 1, 2, and 3;
R⁵ is selected from the group consisting of C₆₋₁₀aryl, and 5-13 membered heteroaryl; wherein the C₆₋₁₀aryl, and 5-13 membered heteroaryl are all optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
each R¹⁴ is independently selected from the group consisting of halogen, —CN, C₁₋₆alkyl, C₁₋₆haloalkyl, and C₃₋₁₀cycloalkyl;
R⁶ is a selected from the group consisting of hydrogen, C₁₋₆alkyl, C₁₋₆haloalkyl, C₃₋₁₀cycloalkyl and 3-11 membered heterocyclyl; wherein C₁₋₆alkyl, C₁₋₆haloalkyl, C₃₋₁₀cycloalkyl and 3-11 membered heterocyclyl are all optionally substituted with one or more, identical or different R¹⁵;
each R¹⁵ is independently selected from the group consisting of halogen, C₁₋₆alkyl, and C₁₋₆haloalkyl;
or a salt thereof.

In another aspect, the invention relates to the compound of the formula (I), or a salt thereof, wherein V and W are nitrogen (—N═).

In another aspect, the invention relates to the compound of the formula (I), or a salt thereof, wherein at least one of V and W is nitrogen (—N═).

In another aspect, the invention relates to the compound of the formula (I), or a salt thereof, wherein
V is —CH═;
W is nitrogen (—N═).

In another aspect, the invention relates to the compound of formula (I), or a salt thereof, wherein
V is nitrogen (—N═),
W is —CH═.

In another aspect, the invention relates to the compound of the formula (I), or a salt thereof, wherein V and W are —CH═.

In another aspect, the invention relates to the compound of the formula (I), or a salt thereof, wherein
U¹ is sulphur (—S—);
U² is —CR⁶═; and
U³ is —CR⁵.

In another aspect, the invention relates to the compound of the formula (I), or a salt thereof, wherein
U¹ is nitrogen (—N═);
U² is nitrogen (—N═); and
U³ is nitrogen (—NR⁵—).

In another aspect, the invention relates to the compound of the formula (I), or a salt thereof, wherein
U¹ is nitrogen (—N═);
U² is —CR⁶═; and
U³ is nitrogen (—NR⁵—).

In another aspect, the invention relates to the compound of the formula (II), or a salt thereof,

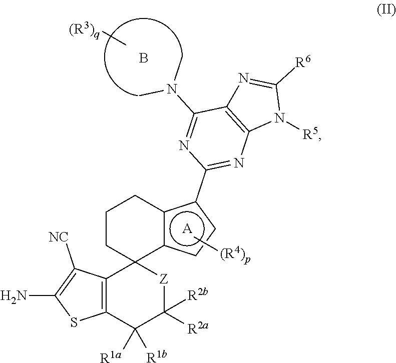

wherein
R^{1a}, R^{1b}, R^{2a}, R^{2b}, R³, R⁴, R⁵, R⁶ Z, ring A, ring B, p and q are as defined herein above or below.

In another aspect, the present invention relates to a compound of the formula (II*) or a salt thereof

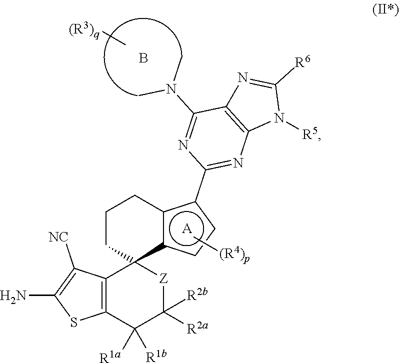

wherein
R^{1a}, R^{1b}, R^{2a}, R^{2b}, Z, R³ to R⁶, ring A, ring B, p, and q are as defined herein above or below.

In another aspect, the invention relates to the compound of the formula (II), or a salt thereof, wherein R^{1a} and R^{1b} are both independently selected from the group consisting of hydrogen and C₁₋₄alkyl.

In another aspect, the invention relates to the compound of the formula (II), or a salt thereof, wherein R^{2a} and R^{2b} are both independently selected from the group consisting of hydrogen and halogen.

In another aspect, the invention relates to the compound of the formula (II), or a salt thereof, wherein R^{1a} and R^{1b} are both independently selected from the group consisting of hydrogen and methyl.

In another aspect, the invention relates to the compound of the formula (II), or a salt thereof, wherein R^{2a} and R^{2b} are both independently selected from the group consisting of hydrogen and fluorine.

In another aspect, the invention relates to the compound of the formula (II), or a salt thereof, wherein R^{1a}, R^{1b}, R^{2a} and R^{2b} are hydrogen.

In another aspect, the invention relates to the compound of the formula (II), or a salt thereof, wherein n is 0.

In another aspect, the invention relates to the compound of the formula (II), or a salt thereof, wherein n is 1; and each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄ alkyl), —N(C₁₋₄alkyl)₂, C₃₋₅cycloalkyl and 3-5 membered heterocyclyl.

In another aspect, the invention relates to the compound of the formula (II), or a salt thereof, wherein Z is —CH₂—.

In another aspect, the invention relates to the compound of the formula (I), or a salt thereof, wherein n is 2; and each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl.

In another aspect, the invention relates to the compound of the formula (II), or a salt thereof, wherein p is 0.

In another aspect the present invention relates to a compound of formula (IIa) or a salt thereof

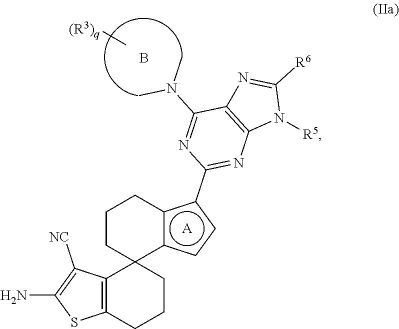

wherein
ring A, ring B, R³, R⁵, R⁶, and q are as defined herein.

In another aspect the present invention relates to a compound of formula (IIb) or a salt thereof

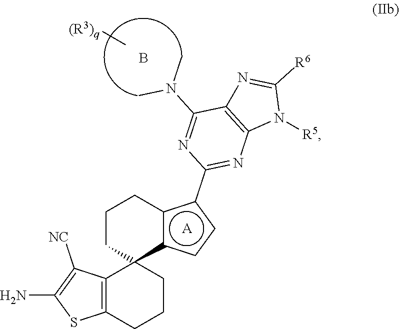

wherein
ring A, ring B, R³, R⁵, R⁶, and q are as defined herein.

In another aspect, the invention relates to the compound of the invention, or a salt thereof, wherein ring A is a ring selected from the group consisting of imidazole, pyrazole, oxazole, isoxazole, thiazole, isothiazole and triazole.

In another aspect, the invention relates to the compound of the invention, or a salt thereof, wherein ring A is a ring selected from the group consisting of pyrrole, furan, thiophene, imidazole, pyrazole, isoxazole, isothiazole and triazole.

In another aspect, the invention relates to the compound of the invention, or a salt thereof, wherein ring A is selected from the group consisting of

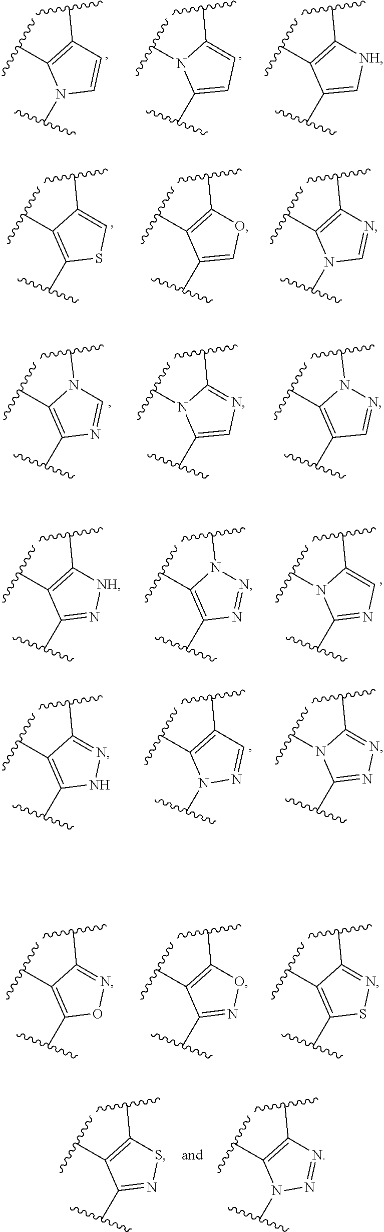

In another aspect, the invention relates to the compound of the invention, or a salt thereof, wherein ring A is isoxazole or isothiazole.

In another aspect, the invention relates to the compound of the invention, or a salt thereof, wherein ring A is selected from

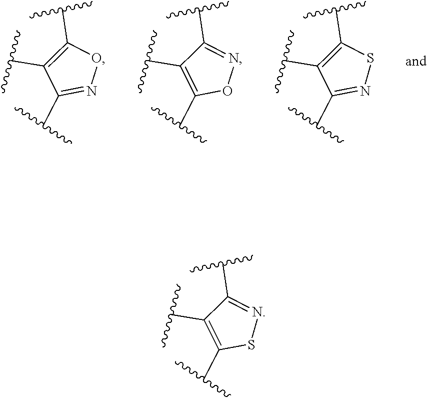 [SMILES: *c1noc(*)c1* ; *c1noc(*)c1* ; *c1nsc(*)c1* ; *c1nsc(*)c1*]

In another aspect, the invention relates to the compound of the invention, or a salt thereof, wherein ring A is

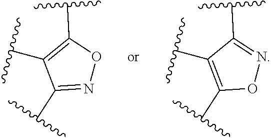 [SMILES: *c1noc(*)c1* ; *c1noc(*)c1*]

In another aspect the invention relates to a compound of formula (IIc) or a salt thereof

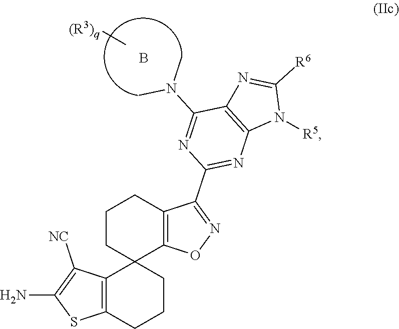

wherein
ring B, R³, R⁵, R⁶, and q are as defined herein.

In another aspect the invention relates to a compound of formula (IId) or a salt thereof

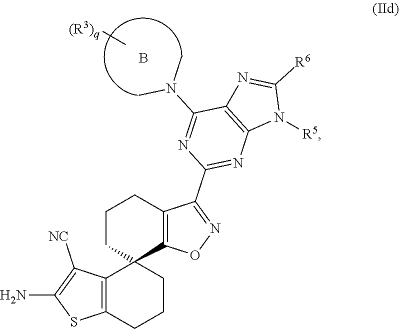

wherein
ring B, R³, R⁵, R⁶, and q are as defined herein.

In another aspect the invention relates to a compound of formula (IIe) or a salt thereof

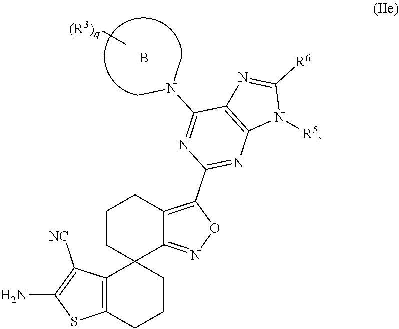

wherein
ring B, R³, R⁵, R⁶, and q are as defined herein.

In another aspect the invention relates to a compound of formula (IIf) or a salt thereof

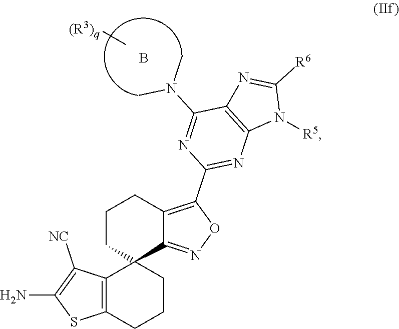

wherein
ring B, R³, R⁵, R⁶, and q are as defined herein.

In another aspect, the invention relates to the compound of the formula (I), (II), (II*), (IIa), (IIb), (IIc), (IId), (IIe) or (IIf), or a salt thereof, wherein ring B is a 4-11 membered heterocycle comprising at least one nitrogen.

In another aspect, the invention relates to the compound of the formula (I), (II), (II*), (IIa), (IIb), (IIc), (IId), (IIe) or (IIf), or a salt thereof, wherein ring B is a 4-11 membered heterocycle comprising at least two nitrogen.

In another aspect, the invention relates to the compound of the formula (I), (II), (II*), (IIa), (IIb), (IIc), (IId), (IIe) or (IIf), or a salt thereof, wherein ring B is a 4-8 membered heterocycle.

In another aspect, the invention relates to the compound of the formula (I), (II), (II*), (IIa), (IIb), (IIc), (IId), (IIe) or (IIf), or a salt thereof, wherein ring B is a 4-8 membered heterocycle comprising at least one nitrogen.

In another aspect, the invention relates to the compound of the formula (I), (II), (II*), (IIa), (IIb), (IIc), (IId), (IIe) or (IIf), or a salt thereof, wherein ring B is a 4-8 membered heterocycle comprising at least two nitrogen.

In another aspect, the invention relates to the compound of the formula (I), (II), (II*), (IIa), (IIb), (IIc), (IId), (IIe) or (IIf), or a salt thereof, wherein ring B is a ring selected from the group consisting of azetidine, pyrrolidine, imidazolidine, piperidine, piperazine, morpholin, azepane, diazepane, oxazepane, 2-azabicyclo[2.2.1]heptane and 4,7-diazaspiro[2.5]octane.

In another aspect, the invention relates to the compound of the formula (I), (II), (II*), (IIa), (IIb), (IIc), (IId), (IIe) or (IIf), or a salt thereof, wherein ring B is a ring selected from the group consisting of azetidine, pyrrolidine, piperazine, 2-azabicyclo[2.2.1]heptane and 4,7-diazaspiro[2.5]octane

In another aspect, the invention relates to the compound of the formula (I), (II), (II*), (IIa), (IIb), (IIc), (IId), (IIe) or (IIf), or a salt thereof, wherein ring B is azetidine.

In another aspect, the invention relates to the compound of the formula (I), (II), (II*), (IIa), (IIb), (IIc), (IId), (IIe) or (IIf), or a salt thereof, wherein ring B is pyrrolidine.

In another aspect, the invention relates to the compound of the formula (I), (II), (II*), (IIa), (IIb), (IIc), (IId), (IIe) or (IIf), or a salt thereof, wherein ring B is piperazine.

In another aspect, the invention relates to the compound of the formula (I), (II), (II*), (IIa), (IIb), (IIc), (IId), (IIe) or (IIf), or a salt thereof, wherein ring B is 2-azabicyclo[2.2.1]heptane.

In another aspect, the invention relates to the compound of the formula (I), (II), (II*), (IIa), (IIb), (IIc), (IId), (IIe) or (IIf), or a salt thereof, wherein each R³, if present, is independently selected from the group consisting of R⁸ and R⁹;
each R⁸ is independently selected from the group consisting of —OR⁹ and —NR⁹R⁹;
each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
each R¹⁰ is independently selected from the group consisting of —OR¹¹, —NR¹¹R¹¹ and halogen;
each R¹¹ is independently selected from the group consisting of C₁₋₆alkyl, C₃₋₁₀cycloalkyl, and 3-11 membered heterocyclyl; wherein the C₁₋₆alkyl, C₃₋₁₀cycloalkyl, and 3-11 membered heterocyclyl are all optionally substituted with one or more identical or different R¹²;
each R¹² is independently selected from the group consisting of halogen, C₁₋₆alkyl, C₁₋₆alkoxy, C₁₋₆haloalkyl.

In another aspect, the invention relates to the compound of the formula (I), (II), (II*), (IIa), (IIb), (IIc), (IId), (IIe) or (IIf), or a salt thereof, wherein
each R³, if present, is independently selected from the group consisting of R⁸ and R⁹;
   each R⁸ is independently selected from the group consisting of —OR⁹ and —NR⁹R⁹;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl.

In another aspect, the invention relates to the compound of the formula (I), (II), (II*), (IIa), (IIb), (IIc), (IId), (IIe) or (IIf), or a salt thereof, wherein q is selected from the group consisting of 0, 1 and 2.

In another aspect, the invention relates to the compound of the formula (I), (II), (II*), (IIa), (IIb), (IIc), (IId), (IIe) or (IIf), or a salt thereof, wherein q is selected from the group consisting of 1 and 2.

In another aspect, the invention relates to the compound of the formula (I), (II), (II*), (IIa), (IIb), (IIc), (IId), (IIe) or (IIf), or a salt thereof, wherein q is of 0.

In another aspect, the invention relates to the compound of the formula (I), (II), (II*), (IIa), (IIb), (IIc), (IId), (IIe) or (IIf), or a salt thereof, wherein q is 1.

In another aspect, the invention relates to the compound of the formula (I), (II), (II*), (IIa), (IIb), (IIc), (IId), (IIe) or (IIf), or a salt thereof, wherein q is 2.

In another aspect, the invention relates to the compound of the formula (I), (II), (II*), (IIa), (IIb), (IIc), (IId), (IIe) or (IIf), or a salt thereof, wherein ring B—(R³)q is selected from the group consisting of

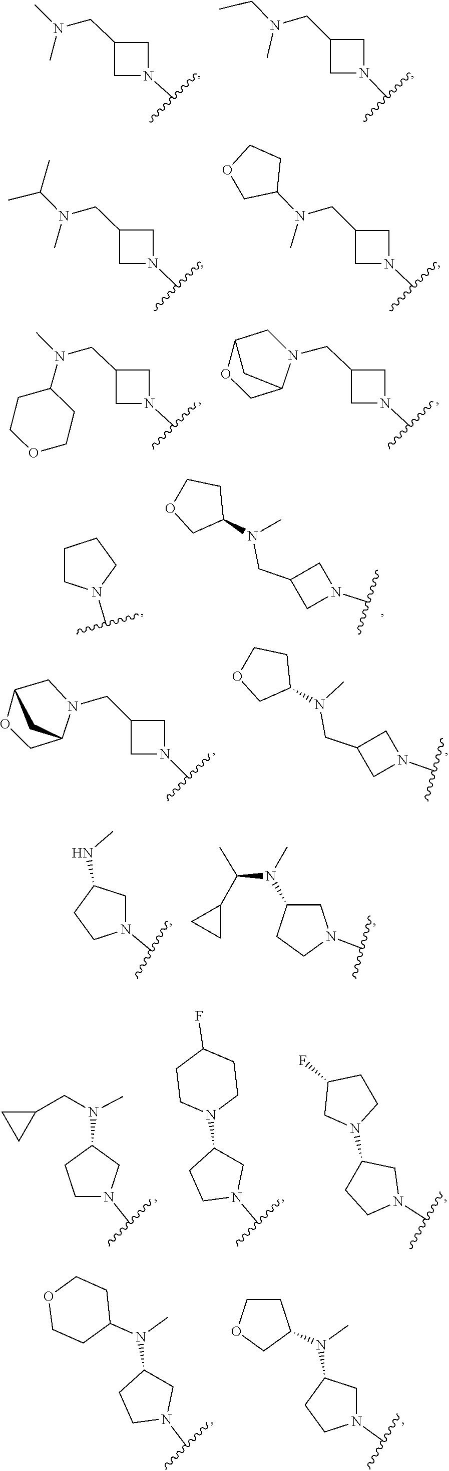

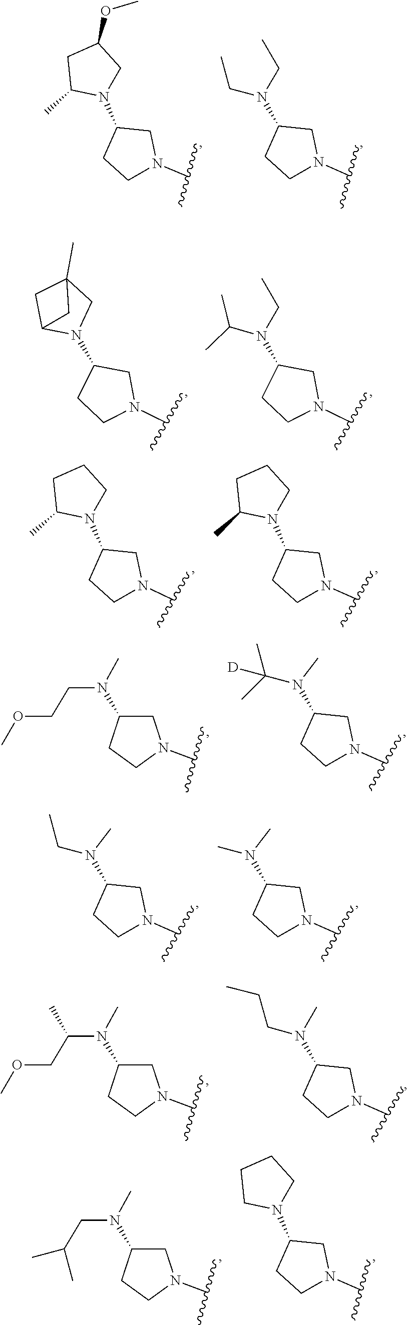

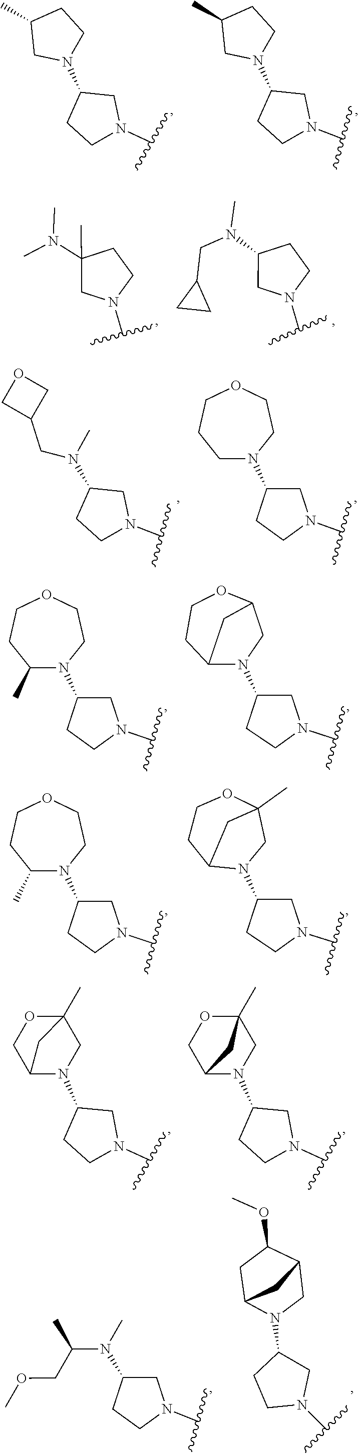

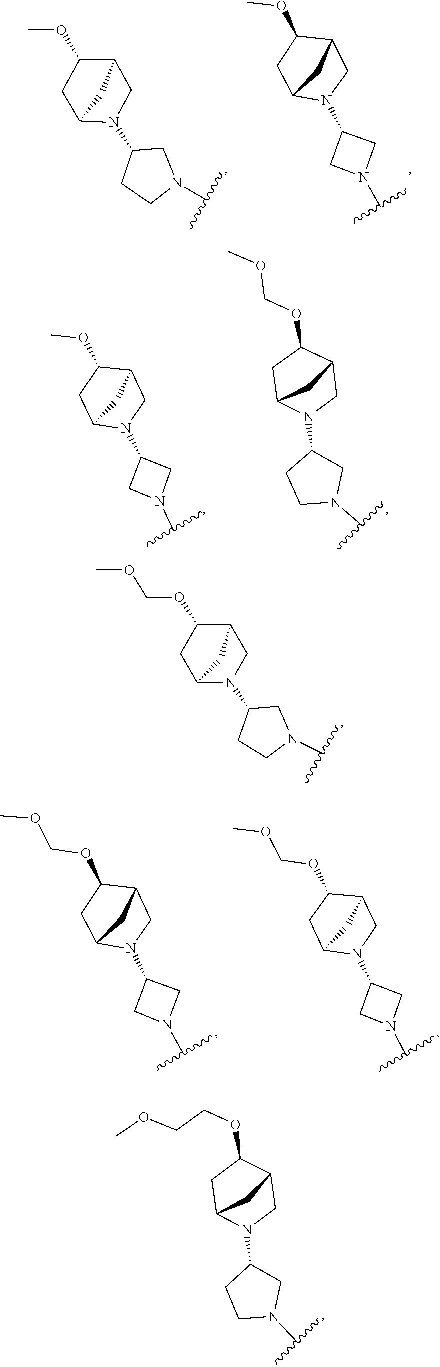

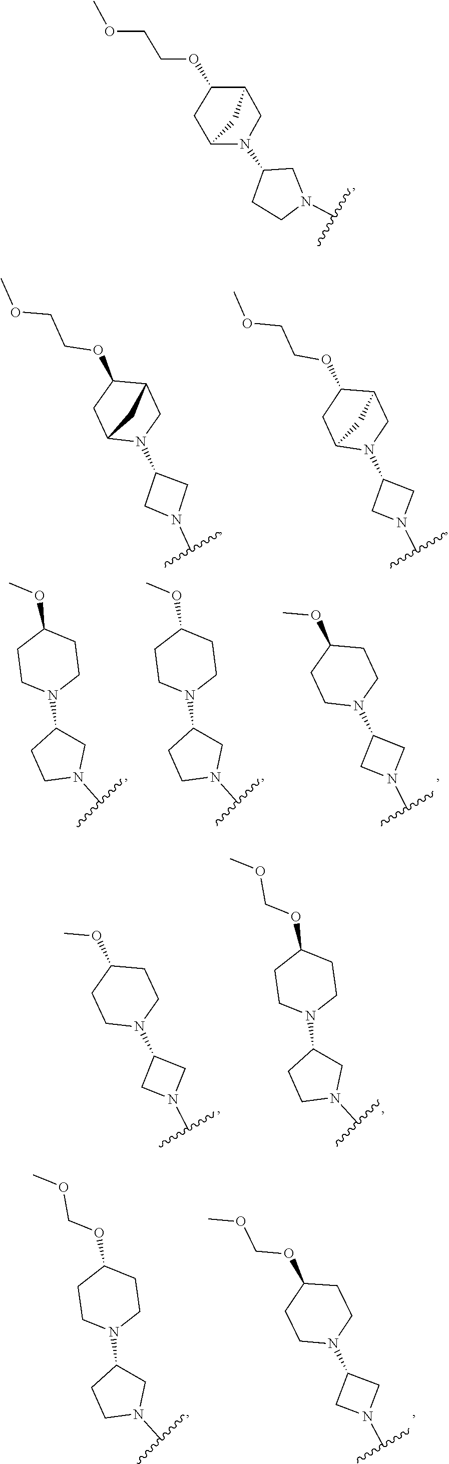

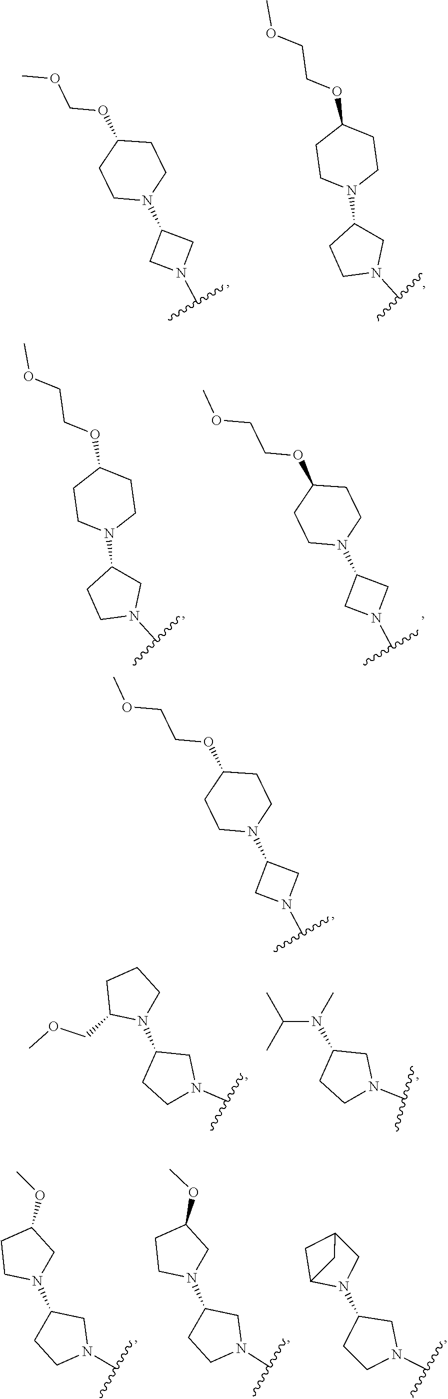

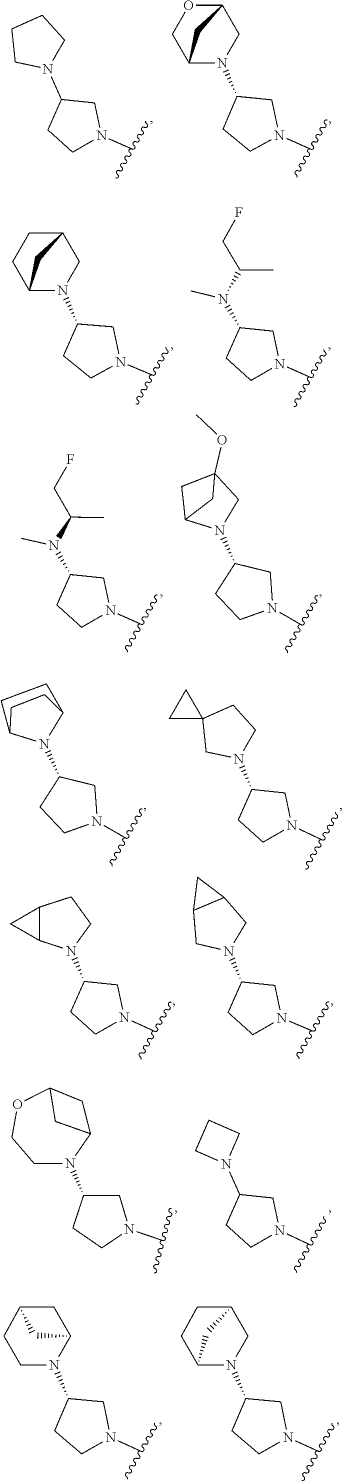

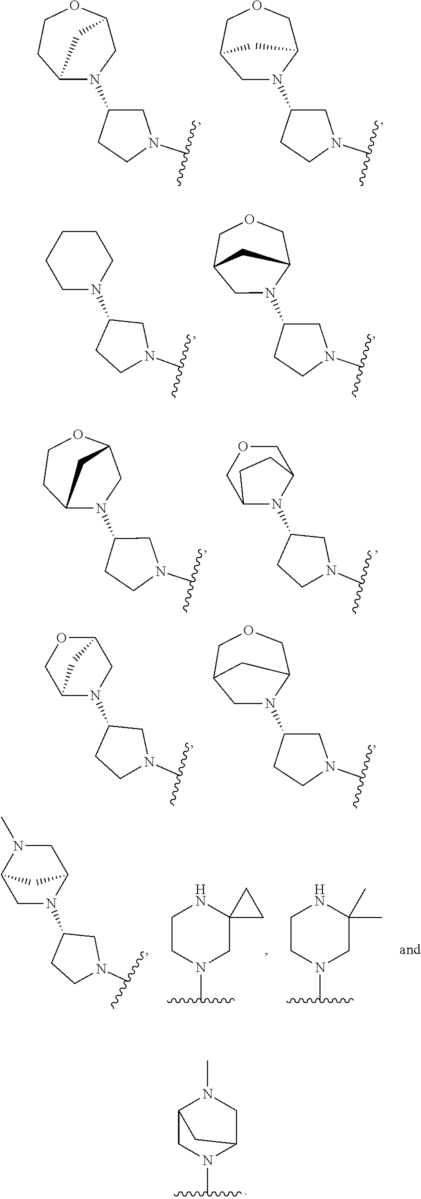

In another aspect, the invention relates to the compound of the formula (I), (II), (II*), (IIa), (IIb), (IIc), (IId), (IIe) or (IIf), or a salt thereof, wherein
R⁵ is C₆₋₁₀aryl, wherein the C₆₋₁₀aryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the invention relates to the compound of the formula (I), (II), (II*), (IIa), (IIb), (IIc), (IId), (IIe) or (IIf), or a salt thereof, wherein
R⁵ is C₆₋₁₀aryl, wherein the C₆₋₁₀aryl is optionally substituted with one or more, identical or different R¹³;
   each R¹³ is independently selected from the group consisting of halogen, —CN, C₁₋₆alkyl, and C₁₋₆haloalkyl; wherein the C₁₋₆alkyl, and C₁₋₆haloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the invention relates to the compound of the formula (I), (II), (II*), (IIa), (IIb), (IIc), (IId), (IIe) or (IIf), or a salt thereof, wherein
R⁵ is phenyl, wherein the phenyl is optionally substituted with one or more, identical or different R¹³;
   each R¹³ is independently selected from the group consisting of halogen, —CN, C₁₋₆alkyl, and C₁₋₆haloalkyl; wherein the C₁₋₆alkyl, and C₁₋₆haloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the invention relates to the compound of the formula (I), (II), (II*), (IIa), (IIb), (IIc), (IId), (IIe) or (IIf), or a salt thereof, wherein
R⁵ is a 5-13 membered heteroaryl; wherein the 5-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the invention relates to the compound of the formula (I), (II), (II*), (IIa), (IIb), (IIc), (IId), (IIe) or (IIf), or a salt thereof, wherein
R⁵ is a 5-6 membered heteroaryl; wherein the 5-6 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the invention relates to the compound of the formula (I), (II), (II*), (IIa), (IIb), (IIc), (IId), (IIe) or (IIf), or a salt thereof, wherein
R⁵ is a 7-13 membered heteroaryl; wherein the 7-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the invention relates to the compound of the formula (I), (II), (II*), (IIa), (IIb), (IIc), (IId), (IIe) or (IIf), or a salt thereof, wherein
R⁵ is a 7-9 membered heteroaryl; wherein the 7-9 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl.

In another aspect, the invention relates to the compound of the formula (I), (II), (II*), (IIa), (IIb), (IIc), (IId), (IIe) or (IIf), or a salt thereof, wherein R⁵ is selected from the group consisting of

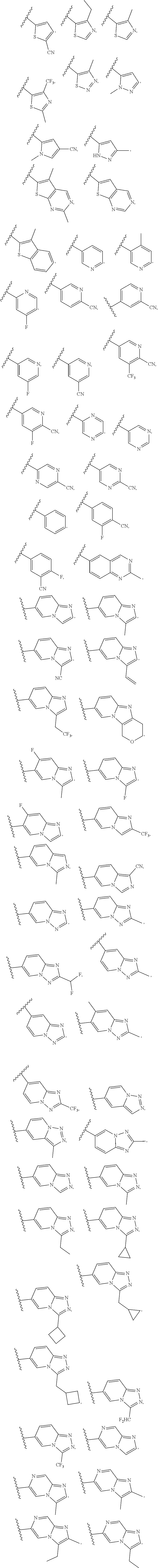

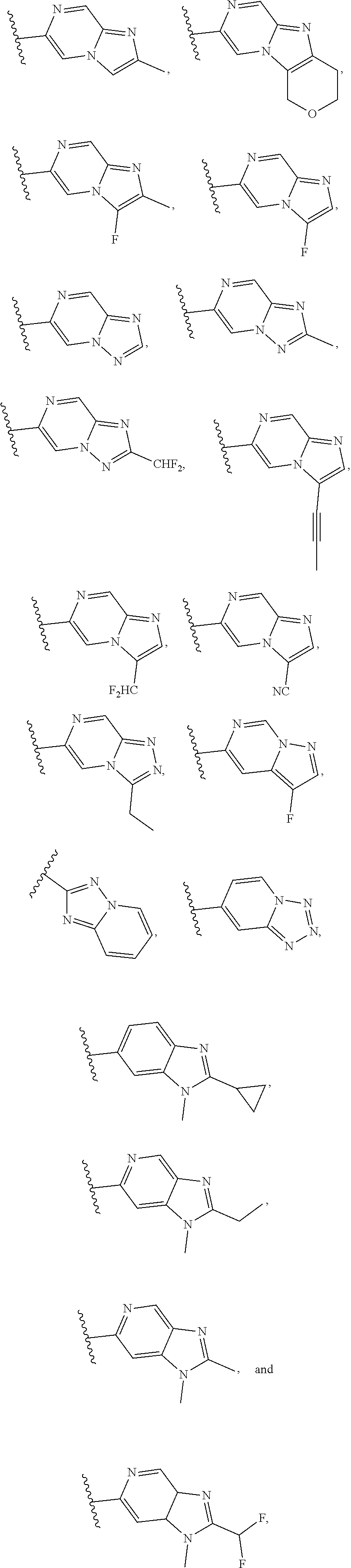

In another aspect, the invention relates to the compound of the formula (I), (II), (II*), (IIa), (IIb), (IIc), (IId), (IIe) or (IIf), or a salt thereof, wherein R⁶ is a selected from the group consisting of C₁₋₆alkyl, C₁₋₆haloalkyl, and C₃₋₁₀cycloalkyl; wherein C₁₋₆alkyl, C₁haloalkyl, and C₃₋₁₀cycloalkyl are all optionally substituted with one or more, identical or different R¹¹;
each R¹⁵ is independently selected from the group consisting of halogen, C₁₋₆alkyl, and C₁₋₆haloalkyl.

In another aspect, the invention relates to the compound of the formula (I), (II), (II*), (IIa), (IIb), (IIc), (IId), (IIe) or (IIf), or a salt thereof, wherein R⁶ is selected from the group consisting of

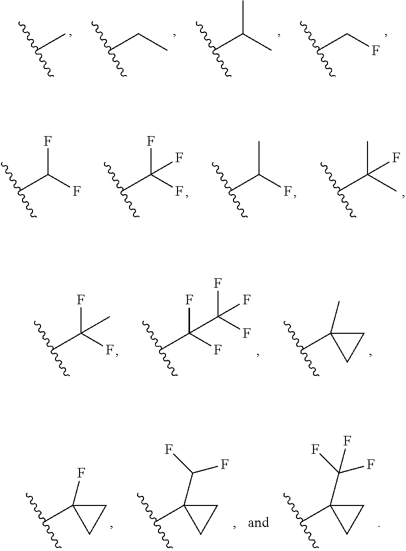

In another aspect, the invention relates to the compound of the formula (IIc), (IId), (IIe) or (IIf), or a salt thereof, wherein
ring B is a 4-8 membered heterocycle;
each R³, if present, is independently selected from the group consisting of R⁸ and R⁹;
   each R⁸ is independently selected from the group consisting of —OR⁹, —NR⁹R⁹, halogen, and —CN;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl;
q is selected from the group consisting of 1 and 2;
R⁵ is a 5-13 membered heteroaryl; wherein the 5-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is independently selected from the group consisting of halogen, —CN, C₁₋₆alkyl, C₁₋₆haloalkyl, and C₃₋₁₀cycloalkyl;
R⁶ is a selected from the group consisting of hydrogen, C₁₋₆alkyl, C₁₋₆haloalkyl, C₃₋₁₀cycloalkyl and 3-11 membered heterocyclyl; wherein C₁₋₆alkyl, C₁₋₆haloalkyl, C₃₋₁₀cycloalkyl and 3-11 membered heterocyclyl are all optionally substituted with one or more, identical or different R¹⁵;
   each R¹⁵ is independently selected from the group consisting of halogen, C₁₋₆alkyl, and C₁₋₆haloalkyl;

In another aspect, the invention relates to the compound of the formula (IIc), (IId), (IIe) or (IIf), or a salt thereof, wherein
ring B is a 4-8 membered heterocycle;
each R³, if present, is independently selected from the group consisting of R⁸ and R⁹;
   each R⁸ is independently selected from the group consisting of —OR⁹, and —NR⁹R⁹;
   each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
   each R¹⁰ is independently selected from the group consisting of —OR¹¹, —NR¹¹R¹¹ and halogen;
   each R¹¹ is independently selected from the group consisting of C₁₋₆alkyl, C₃₋₁₀cycloalkyl, and 3-11 membered heterocyclyl;
   q is selected from the group consisting of 1 and 2;
R⁵ is a 5-13 membered heteroaryl; wherein the 5-13 membered heteroaryl is optionally substituted with one or more, identical or different R¹³;
   each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
   each R¹⁴ is C₃₋₁₀cycloalkyl;
R⁶ is C₁₋₆haloalkyl.

In another aspect, the invention relates to the compound of the formula (IIc), (IId), (IIe) or (IIf), or a salt thereof, wherein
ring B is a 4-8 membered heterocycle;
each R³, if present, is independently selected from the group consisting of R⁸ and R⁹;
   each R⁸ is independently selected from the group consisting of —OR⁹, and —NR⁹R⁹;
   each R⁹ is independently selected from the group consisting of hydrogen, C₁₋₆alkyl, C₁₋₆alkoxy, C₃₋₁₀cycloalkyl, and 3-11 membered heterocyclyl, wherein the C₁₋₆alkyl, C₁₋₆alkoxy, C₃₋₁₀cycloalkyl and 3-11 membered heterocyclyl, are all optionally substituted with one or more, identical or different R¹⁰ and/or R¹¹;
   each R¹⁰ is independently selected from the group consisting of —NR¹¹R¹¹ and halogen;
   each R¹¹ is independently selected from the group consisting of C₁₋₆alkyl, and 3-11 membered heterocyclyl;
q is selected from the group consisting of 1 and 2;
R⁵ is a 5-13 membered heteroaryl; wherein the 5-13 membered heteroaryl is optionally substituted with one or more, identical or different R¹³;
   each R¹³ is independently selected from the group consisting of halogen, —C₁₋₆alkyl, C₁₋₆haloalkyl, and C₃₋₁₀cycloalkyl,
R⁶ is C₁₋₆haloalkyl.

In another aspect, the invention relates to the compound of the formula (IIc), (IId), (IIe) or (IIf), or a salt thereof, wherein
ring B is pyrrolidine;
each R³, if present, is independently selected from the group consisting of R⁸ and R⁹;
   each R⁸ is independently selected from the group consisting of —OR⁹, and —NR⁹R⁹;
   each R⁹ is independently selected from the group consisting of hydrogen, C₁₋₆alkyl, C₁₋₆alkoxy, C₃₋₁₀cycloalkyl, and 3-11 membered heterocyclyl, wherein the C₁₋₆alkyl, C₁₋₆alkoxy, C₃₋₁₀cycloalkyl and 3-11 membered heterocyclyl, are all optionally substituted with one or more, identical or different R¹⁰ and/or R¹¹;
   each R¹⁰ is independently selected from the group consisting of —NR¹¹R¹¹ and halogen;
   each R¹¹ is independently selected from the group consisting of C₁₋₆alkyl, and 3-11 membered heterocyclyl;
q is 1;
R⁵ is a 5-13 membered heteroaryl; wherein the 5-13 membered heteroaryl is optionally substituted with one or more, identical or different R¹³;
   each R¹³ is independently selected from the group consisting of halogen, —C₁₋₆alkyl, C₁₋₆haloalkyl, and C₃₋₁₀cycloalkyl,
R⁶ is C₁₋₆haloalkyl.

Preferred embodiments of the invention are example compounds II-1 to II-197 and any subset thereof.

It is to be understood that any two or more aspects and/or preferred embodiments of formula (I)—or subformulas thereof—may be combined in any way leading to a chemically stable structure to obtain further aspects and/or preferred embodiments of formula (I)—or subformulas thereof.

The present invention further relates to hydrates, solvates, polymorphs, metabolites, derivatives, stereoisomers and prodrugs of a compound of formula (I), (II), (II*), (IIa), (IIb), (IIc), (IId), (IIe) or (IIf), or a salt thereof, (including all embodiments thereof).

The present invention further relates to a hydrate of a compound of formula (I), (II), (II*), (IIa), (IIb), (IIc), (IId), (IIe) or (IIf), or a salt thereof, (including all embodiments thereof).

The present invention further relates to a solvate of a compound of formula (I), (II), (II*), (IIa), (IIb), (IIc), (IId), (IIe) or (IIf), or a salt thereof, (including all embodiments thereof).

Compounds of formula (I), (II), (II*), (IIa), (IIb), (IIc), (IId), (IIe) or (IIf), or a salt thereof, (including all embodiments thereof) which e.g. bear ester groups are potential prodrugs the ester being cleaved under physiological conditions and are also part of the invention.

The present invention further relates to a pharmaceutically acceptable salt of a compound of formula (I), (II), (II*), (IIa), (IIb), (IIc), (IId), (IIe) or (IIf), or a salt thereof, (including all embodiments thereof).

The present invention further relates to a pharmaceutically acceptable salt of a compound of formula (I), (II), (II*), (IIa), (IIb), (IIc), (IId), (IIe) or (IIf), or a salt thereof, (including all embodiments thereof) with an organic or organic acids or bases.

### Pharmaceutical Compositions

A further object of the invention is a pharmaceutical composition comprising a compound of formula (I), (II), (II*), (IIa), (IIb), (IIc), (IId), (IIe) or (IIf), or a salt thereof, or a pharmaceutically acceptable salt thereof—and one or more pharmaceutically acceptable excipient(s).

In one aspect, said pharmaceutical composition optionally comprises one or more other pharmacologically active substance(s). Said one or more other pharmacologically active substance(s) may be the pharmacologically active substances or combination partners as defined herein.

Suitable pharmaceutical compositions for administering the compounds of formula (I), (II), (II*), (IIa), (IIb), (IIc), (IId), (IIe) or (IIf), or a salt thereof, according to the invention will be apparent to those with ordinary skill in the art and include for example tablets, pills, capsules, suppositories, lozenges, troches, solutions, suspensions—particularly solutions, suspensions or other mixtures for injection (s.c., i.v., i.m.) and infusion (injectables)—elixirs, syrups, sachets, emulsions, inhalatives or dispersible powders. The content of the compounds of formula ((I), (II), (II*), (IIa), (IIb), (IIc), (IId), (IIe) or (IIf), or a salt thereof, should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the compounds of formula (I), (II), (II*), (IIa), (IIb), (IIc), (IId), (IIe) or (IIf), or a salt thereof, with known pharmaceutically acceptable excipients, for example inert diluents, carriers, disintegrants, adjuvants, surfactants, binders and/or lubricants. The tablets may also comprise several layers.

Coated tablets may be prepared accordingly by coating cores produced analogously to the tablets with excipients normally used for tablet coatings, for example collidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups or elixirs containing one or more compounds of formula (I), (II), (II*), (IIa), (IIb), (IIc), (IId), (IIe) or (IIf), or a salt thereof, or combinations with one or more other pharmaceutically active substance(s) may additionally contain excipients like a sweetener such as saccharine, cyclamate, glycerol or sugar and a flavour enhancer, e.g. a flavouring such as vanillin or orange extract. They may also contain excipients like suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of excipients like isotonic agents, preservatives such as p-hydroxybenzoates, or stabilisers such as alkali metal salts of ethylenediamine tetra acetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more compounds of formula (I), (II), (II*), (IIa), (IIb), (IIc), (IId), (IIe) or (IIf), or a salt thereof, or combinations with one or more other pharmaceutically active substance(s) may for example be prepared by mixing the compounds/active substance(s) with inert excipients such as lactose or sorbitol and packing them into gelatine capsules.

Suitable suppositories may be made for example by mixing with excipients provided for this purpose such as neutral fats or polyethylene glycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulfate).

The pharmaceutical compositions are administered by the usual methods, preferably by oral or transdermal route, most preferably by oral route. For oral administration the tablets may of course contain, apart from the above-mentioned excipients, additional excipients such as sodium citrate, calcium carbonate and dicalcium phosphate together with various excipients such as starch, preferably potato starch, gelatine and the like. Moreover, lubricants such as magnesium stearate, sodium lauryl sulfate and talc may be used at the same time for the tabletting process. In the case of aqueous suspensions, the active substances may be combined with various flavour enhancers or colourings in addition to the excipients mentioned above.

For parenteral use, solutions of the active substances with suitable liquid excipients may be used.

The dosage range of the compounds of formula (I), (II), (II*), (IIa), (IIb), (IIc), (IId), (IIe) or (IIf), or a salt thereof, applicable per day is usually from 1 mg to 2000 mg, preferably from 250 to 1250 mg.

However, it may sometimes be necessary to depart from the amounts specified, depending on the body weight, age, the route of administration, severity of the disease, the individual response to the drug, the nature of its formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering large amounts, it may be advisable to divide them up into a number of smaller doses spread over the day.

Thus, in a further aspect the invention relates to a pharmaceutical composition comprising at least one (preferably one) compound of formula (I), (II), (II*), (IIa), (IIb), (IIc), (IId), (IIe) or (IIf), or a salt thereof, or a pharmaceutically acceptable salt thereof—and one or more pharmaceutically acceptable excipient(s).

The compounds of formula (I), (II), (II*), (IIa), (IIb), (IIc), (IId), (IIe) or (IIf), or a salt thereof, or the pharmaceutically acceptable salts thereof—and the pharmaceutical compositions comprising such compound and salts may also be co-administered with other pharmacologically active substances, e.g. with other anti-neoplastic compounds (e.g. chemotherapy), i.e. used in combination (see combination treatment further below).

The elements of such combinations may be administered (whether dependently or independently) by methods customary to the skilled person and as they are used in monotherapy, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable excipients appropriate for each route of administration.

Thus, in a further aspect the invention also relates to a pharmaceutical composition comprising a compound of formula (I), (II), (II*), (IIa), (IIb), (IIc), (IId), (IIe) or (IIf), or a salt thereof, or a pharmaceutically acceptable salt thereof—and one or more (preferably one or two, most preferably one) other pharmacologically active substance(s).

In a further aspect the invention also relates to a pharmaceutical preparation comprising a compound of formula (I), (II), (II*), (IIa), (IIb), (IIc), (IId), (IIe) or (IIf), or a salt thereof, or a pharmaceutically acceptable salt thereof—and one or more (preferably one or two, most preferably one) other pharmacologically active substance(s).

Pharmaceutical compositions to be co-administered or used in combination can also be provided in the form of a kit.

Thus, in a further aspect the invention also relates to a kit comprising
a first pharmaceutical composition or dosage form comprising a compound of formula (I), (II), (II*), (IIa), (IIb), (IIc), (IId), (IIe) or (IIf), or a salt thereof, and, optionally, one or more pharmaceutically acceptable excipient(s), and
a second pharmaceutical composition or dosage form comprising another pharmacologically active substance and, optionally, one or more pharmaceutically acceptable excipient(s).

In one aspect such kit comprises a third pharmaceutical composition or dosage form comprising still another pharmacologically active substance and, optionally, one or more pharmaceutically acceptable excipient(s).

### Medical Uses—Methods of Treatment

### Indications—Patient Populations

The present invention is directed to compounds inhibiting KRAS, preferably KRAS mutated at residue 12, such inhibitors selective for KRAS G12D. In particular, compounds of formula (I) (including all embodiments thereof) are potentially useful in the treatment and/or prevention of diseases and/or conditions mediated by KRAS, preferably by KRAS mutated at residue 12, preferably KRAS G12D.

Thus, in a further aspect the invention relates to a compound of formula (I), (II), (II*), (IIa), (IIb), (IIc), (IId), (IIe) or (IIf), or a pharmaceutically acceptable salt thereof—for use as a medicament.

In a further aspect the invention relates to a compound of formula (I), (II), (II*), (IIa), (IIb), (IIc), (IId), (IIe) or (IIf), or a pharmaceutically acceptable salt thereof—for use in a method of treatment of the human or animal body.

In a further aspect the invention relates to a compound of formula (I), (II), (II*), (IIa), (IIb), (IIc), (IId), (IIe) or (IIf), or a pharmaceutically acceptable salt thereof—for use in the treatment and/or prevention of a disease and/or condition mediated by KRAS, preferably by KRAS mutated at residue 12, preferably KRAS G12D.

In a further aspect the invention relates to the use of a compound of formula (I), (II), (II*), (IIa), (IIb), (IIc), (IId), (IIe) or (IIf), or a pharmaceutically acceptable salt thereof—in the manufacture of a medicament for the treatment and/or prevention of a disease and/or condition mediated by KRAS, preferably by KRAS mutated at residue 12, preferably KRAS G12D.

In a further aspect the invention relates to a method for the treatment and/or prevention of a disease and/or condition mediated by KRAS, preferably by KRAS mutated at residue 12, preferably KRAS G12D comprising administering a therapeutically effective amount of a compound of formula (I), (II), (II*), (IIa), (IIb), (IIc), (IId), (IIe) or (IIf), or a pharmaceutically acceptable salt thereof—to a human being.

In a further aspect the invention relates to a compound of formula ((I), (II), (II*), (IIa), (IIb), (IIc), (IId), (IIe) or (IIf), or a pharmaceutically acceptable salt thereof—for use in the treatment and/or prevention of cancer.

In a further aspect the invention relates to a compound of formula (I), (II), (II*), (IIa), (IIb), (IIc), (IId), (IIe) or (IIf), or a pharmaceutically acceptable salt thereof—for use in a method of treatment and/or prevention of cancer in the human or animal body.

In a further aspect the invention relates to the use of a compound of formula (I), (II), (II*), (IIa), (IIb), (IIc), (IId), (IIe) or (IIf), or a pharmaceutically acceptable salt thereof—in the manufacture of a medicament for the treatment and/or prevention of cancer.

In a further aspect the invention relates to a method for the treatment and/or prevention of cancer comprising administering a therapeutically effective amount of a compound of formula (I), (II), (II*), (IIa), (IIb), (IIc), (IId), (IIe) or (IIf), or a pharmaceutically acceptable salt thereof—to a human being.

Preferably, the cancer as defined herein (above or below) comprises a KRAS aberration. In particular, KRAS aberrations include e.g. aberrations of the KRAS gene and/or of the KRAS protein, such as overexpressed KRAS, amplified KRAS or KRAS, KRAS mutated at residue 12, KRAS mutated at residue 13, KRAS mutated at residue 61, KRAS mutated at residue 146, in particular KRAS G12A, KRAS G12C, KRAS G12D, KRAS G12V, KRAS G12S, KRAS G13C, KRAS G13D, KRAS G13V, KRAS Q61H, KRAS Q61E, KRAS Q61P, KRAS A146P, KRAS A146T, KRAS A146V. KRAS may present one or more of these mutations/alterations.

Preferably, the cancer as defined herein (above or below) comprises a BRAF aberration in addition to the KRAS aberration. Said BRAF aberration is in particular a class Ill BRAF mutation, e.g. as defined in Z. Yao, Nature, 2017, 548, 234-238.

In a further aspect the invention relates to a compound of formula (I), (II), (II*), (IIa), (IIb), (IIc), (IId), (IIe) or (IIf), or a pharmaceutically acceptable salt thereof—for use in the treatment and/or prevention of cancer, wherein the cancer comprises a KRAS aberration, said KRAS aberration being preferably KRAS G12D.

In a further aspect the invention relates to the use of a compound of formula (I), (II), (II*), (IIa), (IIb), (IIc), (IId), (IIe) or (IIf), or a pharmaceutically acceptable salt thereof—in the manufacture of a medicament for the treatment and/or prevention of cancer, wherein the cancer comprises a KRAS mutation, said KRAS mutation being preferably KRAS G12D.

In a further aspect the invention relates to a method for the treatment and/or prevention of cancer comprising administering a therapeutically effective amount of a compound of formula (I), (II), (II*), (IIa), (IIb), (IIc), (IId), (IIe) or (IIf), or a pharmaceutically acceptable salt thereof—to a human being, wherein the cancer comprises a KRAS mutation, said KRAS mutation being preferably KRAS G12D.

In a further aspect the invention relates to a compound of formula (I), (II), (II*), (IIa), (IIb), (IIc), (IId), (IIe) or (IIf), or a pharmaceutically acceptable salt thereof—for use in the treatment and/or prevention of cancer, wherein the cancer comprises a KRAS G12D mutation.

In a further aspect the invention relates to a compound of the invention for use in a method of inhibiting KRAS, wherein KRAS can be aberrant, mutated, or overexpressed as defined herein.

In a further aspect the invention relates to the use of a compound of the invention in the manufacture of a medicament for use in a method of inhibiting KRAS, wherein KRAS can be aberrant, mutated or overexpressed as herein defined.

In a further aspect the invention relates to a method for inhibiting KRAS, wherein KRAS can be aberrant, mutated or overexpressed as herein defined, comprising administering a therapeutically effective amount of a compound of the invention to a human being.

According to one aspect, there is provided a method for selecting a patient for treatment with a compound of formula (I), (II), (II*), (IIa), (IIb), (IIc), (IId), (IIe) or (IIf), the method comprising
providing a tumor cell-containing sample from a patient;
determining whether the KRAS gene in the patient's tumor cell-containing sample encodes for mutant (aspartic acid, valine, at position 12) KRAS protein; and
selecting a patient for treatment with a compound of formula (I), (II), (II*), (IIa), (IIb), (IIc), (IId), (IIe) or (IIf), based thereon.

The method may include or exclude the actual patient sample isolation step.

According to another aspect, there is provided a compound of formula (I), (II), (II*), (IIa), (IIb), (IIc), (IId), (IIe) or (IIf), or a pharmaceutically acceptable salt thereof—for use in treating a cancer with tumor cells harbouring a KRAS mutation.

According to another aspect, there is provided a compound of formula (I), (II), (II*), (IIa), (IIb), (IIc), (IId), (IIe) or (IIf), or a pharmaceutically acceptable salt thereof—for use in treating a cancer with tumor cells harbouring a G12D mutant.

According to another aspect, there is provided a compound of (I), (II), (II*), (IIa), (IIb), (IIc), (IId), (IIe) or (IIf), or a pharmaceutically acceptable salt thereof—for use in treating a cancer with tumor cells harbouring a G12D mutant.

According to another aspect, there is provided a compound of formula (I), (II), (II*), (IIa), (IIb), (IIc), (IId), (IIe) or (IIf), or a pharmaceutically acceptable salt thereof—for use in treating a cancer with tumor cells harbouring a G12D mutant KRAS gene.

According to another aspect, there is provided a method of treating a cancer with tumor cells harbouring a G12D mutant KRAS gene comprising administering an effective amount of a compound of formula (I), (II), (II*), (IIa), (IIb), (IIc), (IId), (IIe) or (IIf), or a pharmaceutically acceptable salt thereof—to a human being.

According to another aspect, there is provided a method of treating a cancer with tumor cells harbouring a G12D mutant comprising administering an effective amount of a compound of formula (I), (II), (II*), (IIa), (IIb), (IIc), (IId), (IIe) or (IIf), or a pharmaceutically acceptable salt thereof.

Methods for detecting a mutation in a KRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12D KRAS mutations by real-time PCR. In real-time PCR, fluorescent probes specific for the KRAS G12D mutation are used. When a mutation is present, the probe binds and fluorescence is detected. In some embodiments, the KRAS G12D mutation is identified using a direct sequencing method of specific regions (e.g. exon 2 and/or exon 3) in the KRAS gene. This technique will identify all possible mutations in the region sequenced. Methods for detecting a mutation in a KRAS protein are known by those of skill in the art. These methods include, but are not limited to, detection of a KRAS mutant using a binding agent (e.g. an antibody) specific for the mutant protein, protein electrophoresis, Western blotting and direct peptide sequencing.

Methods for determining whether a tumor or cancer comprises a KRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA. In some embodiments the sample is a liquid biopsy and the test is done on a sample of blood to look for cancer cells from a tumor that are circulating in the blood or for pieces of DNA from tumor cells that are in the blood.

Preferably, the disease/condition/cancer/tumors/cancer cells to be treated/prevented with a compound of formula (I), (II), (II*), (IIa), (IIb), (IIc), (IId), (IIe) or (IIf), or a pharmaceutically acceptable salt thereof—according to the methods and uses as herein (above and below) defined and disclosed is selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute lymphoblastic leukemia, acute myeloid leukaemia, bladder cancer, urothelial cancer, gastric cancer, cervical cancer, head and neck squamous cell carcinoma, diffuse large B cell lymphoma, oesophageal cancer, gastroesophageal cancer, chronic lymphocytic leukaemia, hepatocellular cancer, breast cancer, ovarian cancer, prostate cancer, glioblastoma, renal cancer and sarcomas.

Preferably, the disease/condition/cancer/tumors/cancer cells to be treated/prevented with a compound of formula (I), (II), (II*), (IIa), (IIb), (IIc), (IId), (IIe) or (IIf), or a pharmaceutically acceptable salt thereof—according to the methods and uses as herein (above and below) defined and disclosed is selected from the group consisting of pancreatic cancer, lung cancer, ovarian cancer, colorectal cancer (CRC), gastric cancer, gastroesophageal junction cancer (GEJC) and esophageal cancer. In another aspect, the disease/condition/cancer/tumors/cancer cells to be treated/prevented with a compound of formula (I), (II), (II*), (IIa), (IIb), (IIc), (IId), (IIe) or (IIf), or a pharmaceutically acceptable salt thereof—according to the methods and uses as herein (above and below) defined and disclosed is selected from the group consisting of pancreatic cancer (preferably pancreatic ductal adenocarcinoma (PDAC)), lung cancer (preferably non-small cell lung cancer (NSCLC)), gastric cancer, cholangiocarcinoma and colorectal cancer (preferably colorectal adenocarcinoma). Preferably, said pancreatic cancer, lung cancer, cholangiocarcinoma, colorectal cancer (CRC), pancreatic ductal adenocarcinoma (PDAC), non-small cell lung cancer (NSCLC) or colorectal adenocarcinoma comprises a KRAS mutation, in particular a KRAS G12D mutation. Preferably (in alternative or in combination with the previous preferred embodiment), said non-small cell lung cancer (NSCLC) comprises a mutation (in particular a loss-of-function mutation) in the NF1 gene.

In another aspect, the disease/condition/cancer/tumors/cancer cells to be treated/prevented with a compound of formula (I), (II), (II*), (IIa), (IIb), (IIc), (IId), (IIe) or (IIf), or a pharmaceutically acceptable salt thereof—according to the methods and uses as herein (above and below) defined and disclosed is gastric cancer, ovarian cancer, colorectal cancer (CRC), or esophageal cancer, said gastric cancer or esophageal cancer being preferably selected from the group consisting of: gastric adenocarcinoma (GAC), esophageal adenocarcinoma (EAC) and gastroesophageal junction cancer (GEJC). Preferably, said gastric cancer, ovarian cancer, esophageal cancer, gastric adenocarcinoma (GAC), esophageal adenocarcinoma (EAC) or gastroesophageal junction cancer (GEJC) comprises a KRAS mutation or wildtype amplified KRAS.

Particularly preferred, the cancer to be treated/prevented with a compound of formula (I), (I), (II), (II*), (IIa), (IIb), (IIc), (IId), (IIe) or (IIf), or a pharmaceutically acceptable salt thereof—according to the methods and uses as herein (above and below) defined and disclosed is selected from the group consisting of:
lung adenocarcinoma (preferably non-small cell lung cancer (NSCLC)) harbouring a KRAS mutation at position 12 (preferably a G12D);
colorectal adenocarcinoma harbouring a KRAS mutation at position 12 (preferably a G12D);
pancreatic adenocarcinoma (preferably pancreatic ductal adenocarcinoma (PDAC)) harbouring a RAS mutation at position 12 (preferably a KRAS and preferably a G12D).

Preferably, “cancer” as used herein (above or below) includes drug-resistant cancer and cancer that has failed one, two or more lines of mono- or combination therapy with one or more anti-cancer agents. In particular, “cancer” (and any embodiment thereof) refers to any cancer (especially the cancer species defined hereinabove and hereinbelow) that is resistant to treatment with a KRAS inhibitor, such as KRAS G12C inhibitor.

Different resistance mechanisms have already been reported. For example, the following articles describe resistance in patients following treatment with a KRAS G12C inhibitor: (i) Awad M M, Liu S, Rybkin, II, Arbour K C, Dilly J, Zhu V W, et al. Acquired resistance to KRAS(G12C) inhibition in cancer. N Engl J Med 2021; 384:2382-93 and (ii) Tanaka N, Lin J J, Li C, Ryan M B, Zhang J, Kiedrowski L A, et al. Clinical acquired resistance to KRAS(G12C) inhibition through a novel KRAS switch-II pocket mutation and polyclonal alterations converging on RAS-MAPK reactivation. Cancer Discov 2021; 11:1913-22.

Additionally, the following cancers, tumors and other proliferative diseases may be treated with compounds of formula (I), (II), (II*), (IIa), (IIb), (IIc), (IId), (IIe) or (IIf), or a pharmaceutically acceptable salt thereof—without being restricted thereto:
cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus (e.g. esophageal cancer, gastroesophageal junction cancer), stomach (e.g. gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas, gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP);
All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas;
The compounds of the invention may be used in therapeutic regimens in the context of first line, second line, or any further line treatments.

The compounds of the invention may be used for the prevention, short-term or long-term treatment of the above-mentioned diseases/conditions/cancers/tumors, optionally also in combination with radiotherapy and/or surgery.

The methods of treatment, methods, uses and compounds for use as disclosed herein (above and below) can be performed with any compound of formula (I), (II), (II*), (IIa), (IIb), (IIc), (IId), (IIe) or (IIf), or a pharmaceutically acceptable salt thereof—as disclosed or defined herein and with any pharmaceutical composition or kit comprising a compound of formula (I), (II), (II*), (IIa), (IIb), (IIc), (IId), (IIe) or (IIf), or a pharmaceutically acceptable salt thereof (each including all individual embodiments or generic subsets of compounds of formula (I), (II), (II*), (IIa), (IIb), (IIc), (IId), (IIe) or (IIf).

### Combination Treatment

The compounds of formula (I), (II), (II*), (IIa), (IIb), (IIc), (IId), (IIe) or (IIf), or the pharmaceutically acceptable salts thereof—and the pharmaceutical compositions comprising such compounds or salts may also be co-administered with other pharmacologically active substances, e.g. with other anti-neoplastic compounds (e.g. chemotherapy), or used in combination with other treatments, such as radiation or surgical intervention, either as an adjuvant prior to surgery or post-operatively. Preferably, the pharmacologically active substance(s) for co-administration is/are (an) anti-neoplastic compound(s).

Thus, in a further aspect the invention relates to a compound of (I), (II), (II*), (IIa), (IIb), (IIc), (IId), (IIe) or (IIf), or a pharmaceutically acceptable salt thereof—for use as hereinbefore defined wherein said compound is administered before, after or together with one or more other pharmacologically active substance(s).

In a further aspect the invention relates to a compound of formula (I), (II), (II*), (IIa), (IIb), (IIc), (IId), (IIe) or (IIf), or a pharmaceutically acceptable salt thereof—for use as hereinbefore defined, wherein said compound is administered in combination with one or more other pharmacologically active substance(s).

In a further aspect the invention relates to the use of a compound of formula (I), (II), (II*), (IIa), (IIb), (IIc), (IId), (IIe) or (IIf), or a pharmaceutically acceptable salt thereof—as hereinbefore defined wherein said compound is to be administered before, after or together with one or more other pharmacologically active substance(s).

In a further aspect the invention relates to a method (e.g. a method for the treatment and/or prevention) as hereinbefore defined wherein the compound of formula (I), (II), (II*), (IIa), (IIb), (IIc), (IId), (IIe) or (IIf), or a pharmaceutically acceptable salt thereof—is administered before, after or together with a therapeutically effective amount of one or more other pharmacologically active substance(s).

In a further aspect the invention relates to a method (e.g. a method for the treatment and/or prevention) as hereinbefore defined wherein the compound of formula (I), (II), (II*), (IIa), (IIb), (IIc), (IId), (IIe) or (IIf), or a pharmaceutically acceptable salt thereof—is administered in combination with a therapeutically effective amount of one or more other pharmacologically active substance(s).

In a further aspect the invention relates to a method for the treatment and/or prevention of cancer comprising administering to a patient in need thereof a therapeutically effective amount of a compound of formula (I), (II), (II*), (IIa), (IIb), (IIc), (IId), (IIe) or (IIf), or a pharmaceutically acceptable salt thereof—and a therapeutically effective amount of one or more other pharmacologically active substance(s), wherein the compound of formula (I), (II), (II*), (IIa), (IIb), (IIc), (IId), (IIe) or (IIf), or a pharmaceutically acceptable salt thereof—is administered simultaneously, concurrently, sequentially, successively, alternately or separately with the one or more other pharmacologically active substance(s).

In a further aspect the invention relates to a compound of formula (I), (II), (II*), (IIa), (IIb), (IIc), (IId), (IIe) or (IIf), or a pharmaceutically acceptable salt thereof—for use in the treatment and/or prevention of cancer, wherein the compound of formula (I), (II), (II*), (IIa), (IIb), (IIc), (IId), (IIe) or (IIf), or a pharmaceutically acceptable salt thereof—is administered simultaneously, concurrently, sequentially, successively, alternately or separately with the one or more other pharmacologically active substance(s).

In a further aspect the invention relates to a kit comprising
a first pharmaceutical composition or dosage form comprising a compound of formula (I), (II), (II*), (IIa), (IIb), (IIc), (IId), (IIe) or (IIf), or a pharmaceutically acceptable salt thereof—and, optionally, one or more pharmaceutically acceptable excipient(s), and
a second pharmaceutical composition or dosage form comprising another pharmacologically active substance, and, optionally, one or more pharmaceutically acceptable excipient(s),
   for use in the treatment and/or prevention of cancer, wherein the first pharmaceutical composition is to be administered simultaneously, concurrently, sequentially, successively, alternately or separately with the second and/or additional pharmaceutical composition or dosage form.

In one aspect such kit for said use comprises a third pharmaceutical composition or dosage form comprising a third pharmaceutical composition or dosage form comprising still another pharmacologically active substance, and, optionally, one or more pharmaceutically acceptable excipient(s)

In a further embodiment of the invention the components (i.e. the combination partners) of the combinations, kits, uses, methods and compounds for use according to the invention (including all embodiments) are administered simultaneously.

In a further embodiment of the invention the components (i.e. the combination partners) of the combinations, kits, uses, methods and compounds for use according to the invention (including all embodiments) are administered concurrently.

In a further embodiment of the invention the components (i.e. the combination partners) of the combinations, kits, uses, methods and compounds for use according to the invention (including all embodiments) are administered sequentially.

In a further embodiment of the invention the components (i.e. the combination partners) of the combinations, kits, uses, methods and compounds for use according to the invention (including all embodiments) are administered successively.

In a further embodiment of the invention the components (i.e. the combination partners) of the combinations, kits, uses, methods and compounds for use according to the invention (including all embodiments) are administered alternately.

In a further embodiment of the invention the components (i.e. the combination partners) of the combinations, kits, uses, methods and compounds for use according to the invention (including all embodiments) are administered separately.

The pharmacologically active substance(s) to be used together/in combination with the compound of formula (I) or a pharmaceutically acceptable salt thereof—(including all individual embodiments or generic subsets of compounds) or in the medical uses, uses, methods of treatment and/or prevention, pharmaceutical compositions as herein (above and below) defined can be selected from any one or more of the following (preferably there is one or two additional pharmacologically active substance used in all these embodiments):
1. an inhibitor of EGFR and/or ErbB2 (HER2) and/or ErbB3 (HER3) and/or ErbB4 (HER4) or of any mutants thereof
   a. irreversible inhibitors: e.g. afatinib, dacomitinib, canertinib, neratinib, avitinib, poziotinib, AV 412, PF-6274484, HKI 357, olmutinib, osimertinib, almonertinib, nazartinib, lazertinib, pelitinib, zongertinib;
   b. reversible inhibitors: e.g. erlotinib, gefitinib, icotinib, sapitinib, lapatinib, varlitinib, vandetanib, TAK-285, AEE788, BMS599626/AC-480, GW 583340;
   c. anti-EGFR antibodies: e.g. necitumumab, panitumumab, cetuximab, amivantamab;
   d. anti-HER2 antibodies: e.g. pertuzumab, trastuzumab,
   e. HER2 ADC (antibody drug conjugate): e.g. trastuzumab emtansine, trastuzumab deruxtecan;
   f. inhibitors of mutant EGFR;
   g. an inhibitor of HER2 with exon 20 mutations; e.g. zongertinib;
   h. preferred irreversible inhibitor is afatinib;
   i. preferred anti-EGFR antibody is cetuximab.
2. an inhibitor of MEK and/or of mutants thereof
   a. e.g. trametinib, cobimetinib, binimetinib, selumetinib, refametinib;
   b. preferred is trametinib
   c. a MEK inhibitor as disclosed in WO 2013/136249;
   d. a MEK inhibitor as disclosed in WO 2013/136254
3. an inhibitor of SOS1 and/or of any mutants thereof (i.e. a compound that modulates/inhibits the GEF functionality of SOS1, e.g. by binding to SOS1 and preventing protein-protein interaction between SOS1 and a (mutant) Ras protein, e.g. KRAS)
   a. e.g. BAY-293;
   b. a SOS1 inhibitor as disclosed in WO 2018/115380;
   c. a SOS1 inhibitor as disclosed in WO 2019/122129;
   d. a SOS1 inhibitor as disclosed in WO 2020/180768, WO 2020/180770, WO 2018/172250, WO 2019/201848, WO2022/146698 and WO2023/118250.
4. an inhibitor of Ras protein and/or of any mutants thereof
   a. a. e.g. a RAS inhibitor
   b. b. preferred RMC-6236
   c. c. e.g. a KRAS inhibitor
   d. preferred a KRAS G12C or G12D inhibitor
5. an inhibitor of YAP1, WWTR1, TEAD1, TEAD2, TEAD3 and/or TEAD4
   a. reversible inhibitors of TEAD transcription factors (e.g. disclosed in WO 2018/204532);
   b. irreversible inhibitors of TEAD transcription factors (e.g. disclosed in WO 2020/243423);
   c. protein-protein interaction inhibitors of the YAP/TAZ::TEAD interaction (e.g. disclosed in WO 2021/186324);
   d. inhibitors of TEAD palmitoylation.
6. an oncolytic virus
7. a RAS vaccine
   a. e.g. Targovax
8. a cell cycle inhibitor
   a. e.g. inhibitors of CDK4/6 and/or of any mutants therof
      i. e.g. palbociclib, ribociclib, abemaciclib, trilaciclib, ebvaciclib;
      ii. preferred are palbociclib and abemaciclib;
      iii. most preferred is abemaciclib.
   b. e.g. vinca alkaloids
      i. e.g. vinorelbine.
   c. e.g. inhibitors of Aurora kinase and/or of any mutants thereof
      i. e.g. alisertib, barasertib.
9. an inhibitor of PTK2 (=FAK) and/or of any mutants thereof
   a. e.g. TAE226, BI 853520.
10. an inhibitor of SHP2 and/or of any mutants thereof
   a. e.g. SHP099, TNO155, RMC-4550, RMC-4630, IACS-13909.
11. an inhibitor of PI3 kinase (=PI3K) and/or of any mutants thereof
   a. e.g. inhibitors of PI3Kα and/or of any mutants thereof
      i. e.g. alpelisib, serabelisib, Inavolisib, HH-CYH33, AMG 511, buparlisib, dactolisib, pictilisib, taselisib.
12. an inhibitor of FGFR1 and/or FGFR2 and/or FGFR3 and/or of any mutants thereof
   a. e.g. ponatinib, infigratinib, nintedanib.
13. an inhibitor of AXL and/or of any mutants thereof
14. a taxane
   a. e.g. paclitaxel, nab-paclitaxel, docetaxel;
   b. preferred is paclitaxel.
15. a platinum-containing compound
   a. e.g. cisplatin, carboplatin, oxaliplatin
   b. preferred is oxaliplatin.
16. an anti-metabolite
   a. e.g. 5-fluorouracil, capecitabine, floxuridine, cytarabine, gemcitabine, pemetrexed, combination of trifluridine and tipiracil (=TAS102);
   b. preferred is 5-fluorouracil.
17. an immunotherapeutic agent
   a. e.g. an immune checkpoint inhibitor
      i. e.g. an anti-CTLA4 mAb, anti-PD1 mAb, anti-PD-L1 mAb, anti-PD-L2 mAb, anti-LAG3 mAb, anti-TIM3 mAb;
      ii. preferred is an anti-PD1 mAb;
      iii. e.g. ipilimumab, nivolumab, pembrolizumab, tislelizumab atezolizumab, avelumab, durvalumab, pidilizumab, spartalizumab, AMG-404, ezabenlimab;
      iv. preferred are nivolumab, pembrolizumab, ezabenlimab and spartalizumab;
      v. most preferred is ezabenlimab, pembrolizumab and nivolumab.
   b. e.g. T cell engagers
      i. e.g. STING agonists;
      ii. preferred a STING agonist as disclosed in WO2022229341
18. a topoisomerase inhibitor
   a. e.g. irinotecan, liposomal irinotecan (nal-IRI), topotecan, etoposide;
   b. most preferred is irinotecan and liposomal irinotecan (nal-IRI).
19. an inhibitor of A-Raf and/or B-Raf and/or C-Raf and/or of any mutants thereof
   a. e.g. encorafenib, dabrafenib, vemurafenib, PLX-8394, RAF-709 (=example 131 in WO 2014/151616), Naporafenib, sorafenib, LY-3009120 (=example 1 in WO 2013/134243), lifirafenib, TAK-632, agerafenib, CCT196969, Avutometinib, RAF265.
20. an inhibitor of mTOR
   a. e.g. rapamycin, temsirolimus, everolimus, ridaforolimus, zotarolimus, sapanisertib, Torin 1, dactolisib, GDC-0349, VS-5584, vistusertib, AZD8055.
21. an epigenetic regulator
   a. e.g. a BET inhibitor
      i. e.g. JQ-1, Molibresib, OTX-015, Pelabresib, TEN-010, OTX-015, PLX51107, Mivebresib, ABBV-744, BMS986158, TGI-1601, Trotabresib, AZD5153, I-BET151, amredobresib;
22. an inhibitor of IGF1/2 and/or of IGF1-R and/or of any mutants thereof
   a. e.g. xentuzumab (antibody 60833 in WO 2010/066868), dusigitumab, linsitinib.
23. an inhibitor of a Src family kinase and/or of any mutants thereof
   a. e.g. an inhibitor of a kinase of the SrcA subfamily and/or of any mutants thereof, i.e. an inhibitor of Src, Yes, Fyn, Fgr and/or of any mutants thereof;
   b. e.g. an inhibitor of a kinase of the SrcB subfamily and/or of any mutants thereof, i.e. an inhibitor of Lck, Hck, Blk, Lyn and/or of any mutants thereof;
   c. e.g. an inhibitor of a kinase of the Frk subfamily and/or of any mutants thereof, i.e. an inhibitor of Frk and/or of any mutants thereof;
   d. e.g. dasatinib, ponatinib, bosutinib, vandetanib, KX-01, saracatinib, KX2-391, SU 6656, WH-4-023.
24. an apoptosis regulator
   a. e.g. an MDM2 inhibitor, e.g. an inhibitor of the interaction between p53 (preferably functional p53, most preferably wt p53) and MDM2 and/or of any mutants thereof;
      i. e.g. Siremadlin, NVP-CGM097, RG-7112, MK-8242, Idasanutlin, SAR405838, AMG-232, Milademetan, RG-7775, alrizomadlin;
      ii. preferred are HDM-201, RG-7388 and AMG-232;
      iii. an MDM2 inhibitor as disclosed in WO 2015/155332;
      iv. an MDM2 inhibitor as disclosed in WO 2016/001376;
      v. an MDM2 inhibitor as disclosed in WO 2016/026937;
      vi. an MDM2 inhibitor as disclosed in WO 2017/060431;
   b. e.g. a PARP inhibitor;
   c. e.g. an MCL-1 inhibitor;
      i. e.g. AZD-5991, AMG-176, murizatoclax, S64315, S63845, A-1210477;
25. an inhibitor of c-MET and/or of any mutants thereof
   a. e.g. savolitinib, cabozantinib, foretinib;
   b. MET antibodies, e.g. emibetuzumab, amivantamab;
26. an inhibitor of ERK and/or of any mutants thereof
   a. e.g. ulixertinib, LTT462;
27. an inhibitor of farnesyl transferase and/or of any mutants thereof
   a. e.g. tipifarnib;
28. an inhibitor of VEFG and/or of any mutants thereof
   a. e.g. ramucirumab;
29. a bi- or trispecific antibody
   a. bispecific antibody, e.g. EGFR:LGR5 (z.B. petosemtamab), EGFR:Met (z.B. amivantamab), EGFR-HER3, PD1-VEGF.

In a further embodiment of the (combined) use and method (e.g. method for the treatment and/or prevention) as hereinbefore described one other pharmacologically active substance is to be administered before, after or together with the compound of formula (I)—or a pharmaceutically acceptable salt thereof—wherein said one other pharmacologically active substance is
a SOS1 inhibitor; or
a MEK inhibitor; or
trametinib, or
an anti-PD-1 antibody; or
ezabenlimab; or
cetuximab; or
afatinib; or
standard of care (SoC) in a given indication; or
a PI3 kinase inhibitor; or
an inhibitor of TEAD palmitoylation; or
a YAP/TAZ TEAD inhibitor.

In a further embodiment of the (combined) use and method (e.g., method for the treatment and/or prevention) as hereinbefore described one other pharmacologically active substance is to be administered in combination with the compound of formula (I) or a pharmaceutically acceptable salt thereof—wherein said one other pharmacologically active substance is
a SOS1 inhibitor; or
a MEK inhibitor; or
trametinib; or
an anti-PD-1 antibody; or
ezabenlimab; or
cetuximab; or
afatinib; or
standard of care (SoC) in a given indication; or
a PI3 kinase inhibitor; or
an inhibitor of TEAD palmitoylation; or
a YAP/TAZ TEAD inhibitor.

In a further aspect of the (combined) use and method (e.g. method for the treatment and/or prevention) as hereinbefore described two other pharmacologically active substances are to be administered before, after or together with the compound of formula (I)—or a pharmaceutically acceptable salt thereof—wherein said two other pharmacologically active substances are
a MEK inhibitor and a SOS1 inhibitor; or
trametinib and a SOS1 inhibitor; or
an anti-PD-1 antibody (preferably ezabenlimab) and an anti-LAG-3 antibody; or
an anti-PD-1 antibody (preferably ezabenlimab) and a SOS1 inhibitor; or
a MEK inhibitor and an inhibitor selected from the group consisting of an EGFR inhibitor and/or ErbB2 (HER2) inhibitor and/or inhibitor of any mutants thereof; or
a SOS1 inhibitor and an inhibitor selected from the group consisting of an EGFR inhibitor and/or ErbB2 (HER2) inhibitor and/or inhibitor of any mutants thereof; or
a MEK inhibitor and afatinib; or
a MEK inhibitor and cetuximab; or
trametinib and afatinib; or
trametinib and cetuximab; or
a SOS1 inhibitor and afatinib; or
a SOS1 inhibitor and cetuximab; or
a SOS1 inhibitor and an inhibitor of TEAD palmitoylation; or
a SOS1 inhibitor and a YAP/TAZ TEAD inhibitor.

In a further aspect of the (combined) use and method (e.g. method for the treatment and/or prevention) as hereinbefore described two other pharmacologically active substances are to be administered in combination with the compound of formula (I) or a pharmaceutically acceptable salt thereof—wherein said two other pharmacologically active substances are
a MEK inhibitor and a SOS1 inhibitor; or
trametinib and a SOS1 inhibitor; or
an anti-PD-1 antibody (preferably ezabenlimab) and an anti-LAG-3 antibody; or
an anti-PD-1 antibody (preferably ezabenlimab) and a SOS1 inhibitor; or
a MEK inhibitor and an inhibitor selected from the group consisting of an EGFR inhibitor and/or ErbB2 (HER2) inhibitor and/or inhibitor of any mutants thereof; or
a SOS1 inhibitor and an inhibitor selected from the group consisting of an EGFR inhibitor and/or ErbB2 (HER2) inhibitor and/or inhibitor of any mutants thereof; or
a MEK inhibitor and afatinib; or
a MEK inhibitor and cetuximab; or
trametinib and afatinib; or
trametinib and cetuximab; or
a SOS1 inhibitor and afatinib; or
a SOS1 inhibitor and cetuximab; or
a SOS1 inhibitor and an inhibitor of TEAD palmitoylation; or
a SOS1 inhibitor and a YAP/TAZ TEAD inhibitor.

Additional pharmacologically active substance(s) which can also be used together/in combination with the compound of formula (I), (II), (II*), (IIa), (IIb), (IIc), (IId), (IIe) or (IIf), or a pharmaceutically acceptable salt thereof—(including all individual embodiments or generic subsets of compounds of formula (I) or in the medical uses, uses, methods of treatment and/or prevention, pharmaceutical compositions, kits as herein (above and below) defined include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor (HGF) and/or their corresponding receptors), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib, bevacizumab and trastuzumab); antimetabolites (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP inhibitors/SMAC mimetics, Mcl-1, MDM2/MDMX), MEK inhibitors, ERK inhibitors, FLT3 inhibitors, BRD4 inhibitors, IGF-1R inhibitors, TRAILR2 agonists, Bcl-xL inhibitors, Bcl-2 inhibitors (e.g. venetoclax), Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors (e.g. carfilzomib), immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), t-cell engagers (e.g. bi-specific T-cell engagers (BiTEs®) like e.g. CD3×BCMA, CD3×CD33, CD3×CD19), PSMA×CD3), tumor vaccines, immunomodulator, e.g. STING agonist, and various chemotherapeutic agents such as amifostin, anagrelid, clodronat, filgrastin, interferon, interferon alpha, leucovorin, procarbazine, levamisole, mesna, mitotane, pamidronate and porfimer.

It is to be understood that the combinations, compositions, kits, methods, uses, pharmaceutical compositions or compounds for use according to this invention may envisage the simultaneous, concurrent, sequential, successive, alternate or separate administration of the active ingredients or components. It will be appreciated that the compound of formula (I), (II), (II*), (IIa), (IIb), (IIc), (IId), (IIe) or (IIf), or a pharmaceutically acceptable salt thereof—and the one or more other pharmacologically active substance(s) can be administered formulated either dependently or independently, such as e.g. the compound of formula (I), (II), (II*), (IIa), (IIb), (IIc), (IId), (IIe) or (IIf), or a pharmaceutically acceptable salt thereof—and the one or more other pharmacologically active substance(s) may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active ingredient and includes both fixed and non-fixed (e.g. free) combinations (including kits) and uses, such as e.g. the simultaneous, concurrent, sequential, successive, alternate or separate use of the components or ingredients. The term “fixed combination” means that the active ingredients are administered to a patient simultaneously in the form of a single entity or dosage. The term “non-fixed combination” means that the active ingredients are administered to a patient as separate entities either simultaneously, concurrently or sequentially with no specific time limits, wherein such administration provides therapeutically effective levels of the compounds in the body of the patient.

The administration of the compound of formula (I), (II), (II*), (IIa), (IIb), (IIc), (IId), (IIe) or (IIf), or a pharmaceutically acceptable salt thereof—and the one or more other pharmacologically active substance(s) may take place by co-administering the active components or ingredients, such as e.g. by administering them simultaneously or concurrently in one single or in two or more separate formulations or dosage forms. Alternatively, the administration of the compound of formula (I), (II), (II*), (IIa), (IIb), (IIc), (IId), (IIe) or (IIf), or a pharmaceutically acceptable salt thereof—and the one or more other pharmacologically active substance(s) may take place by administering the active components or ingredients sequentially or in alternation, such as e.g. in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to:

The use of the prefix C_{x-y}, wherein x and y each represent a positive integer (x<y), indicates that the chain or ring structure or combination of chain and ring structure as a whole, specified and mentioned in direct association, may consist of a maximum of y and a minimum of x carbon atoms.

The indication of the number of members in groups that contain one or more heteroatom(s) (e.g. heteroaryl, heteroarylalkyl, heterocyclyl, heterocycylalkyl) relates to the total number of atoms of all the ring members or the total of all the ring and carbon chain members.

The indication of the number of carbon atoms in groups that consist of a combination of carbon chain and carbon ring structure (e.g. cycloalkylalkyl, arylalkyl) relates to the total number of carbon atoms of all the carbon ring and carbon chain members. Obviously, a ring structure has at least three members.

In general, for groups comprising two or more subgroups (e.g. heteroarylalkyl, heterocycylalkyl, cycloalkylalkyl, arylalkyl) the last-named subgroup is the radical attachment point, for example, the substituent aryl-C₁₋₆alkyl means an aryl group which is bound to a C₁₋₆alkyl group, the latter of which is bound to the core or to the group to which the substituent is attached.

In groups like HO, H₂N, (O)S, (O)₂S, NC (cyano), HOOC, F₃C or the like, the skilled artisan can see the radical attachment point(s) to the molecule from the free valences of the group itself.

The expression “compound of the invention” and grammatical variants thereof comprises compounds of formula (I), including all salts, aspects and preferred embodiments thereof as herein defined. Any reference to a compound of the invention or to a compound of formula (I) is intended to include a reference to the respective (sub)aspects and embodiments.

Alkyl denotes monovalent, saturated hydrocarbon chains, which may be present in both straight-chain (unbranched) and branched form. If an alkyl is substituted, the substitution may take place independently of one another, by mono- or polysubstitution in each case, on all the hydrogen-carrying carbon atoms.

The term “C₁₋₅alkyl” includes for example H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

Further examples of alkyl are methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃)CH₂CH₃), 2-methyl-2-propyl (tert-butyl; t-Bu; —C(CH₃)₃), 1-pentyl (n-pentyl; —CH₂CH₂CH₂CH₂CH₃), 2-pentyl (—CH(CH₃)CH₂CH₂CH₃), 3-pentyl (—CH(CH₂CH₃)₂), 3-methyl-1-butyl (iso-pentyl; —CH₂CH₂CH(CH₃)₂), 2-methyl-2-butyl (—C(CH₃)₂CH₂CH₃), 3-methyl-2-butyl (—CH(CH₃)CH(CH₃)₂), 2,2-dimethyl-1-propyl (neo-pentyl; —CH₂C(CH₃)₃), 2-methyl-1-butyl (—CH₂CH(CH₃)CH₂CH₃), 1-hexyl (n-hexyl; —CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 2,3-dimethyl-1-butyl (—CH₂CH(CH₃)CH(CH₃)CH₃), 2,2-dimethyl-1-butyl (—CH₂C(CH₃)₂CH₂CH₃), 3,3-dimethyl-1-butyl (—CH₂CH₂C(CH₃)₃), 2-methyl-1-pentyl (—CH₂CH(CH₃)CH₂CH₂CH₃), 3-methyl-1-pentyl (—CH₂CH₂CH(CH₃)CH₂CH₃), 1-heptyl (n-heptyl), 2-methyl-1-hexyl, 3-methyl-1-hexyl, 2,2-dimethyl-1-pentyl, 2,3-dimethyl-1-pentyl, 2,4-dimethyl-1-pentyl, 3,3-dimethyl-1-pentyl, 2,2,3-trimethyl-1-butyl, 3-ethyl-1-pentyl, 1-octyl (n-octyl), 1-nonyl (n-nonyl); 1-decyl (n-decyl) etc.

By the terms propyl, butyl, pentyl, hexyl, heptyl, octyl, nonyl, decyl etc. without any further definition are meant saturated hydrocarbon groups with the corresponding number of carbon atoms, wherein all isomeric forms are included.

The above definition for alkyl also applies if alkyl is a part of another (combined) group such as for example C_{x-y}alkylamino or C_{x-y}alkyloxy.

The term alkylene can also be derived from alkyl. Alkylene is bivalent, unlike alkyl, and requires two binding partners. Formally, the second valency is produced by removing a hydrogen atom in an alkyl. Corresponding groups are for example —CH₃ and —CH₂—, —CH₂CH₃ and —CH₂CH₂— or >CHCH₃ etc.

The term “C₁₋₄alkylene” includes for example —(CH₂)—, —(CH₂—CH₂)—, —(CH(CH₃))—, —(CH₂—CH₂—CH₂)—, —(C(CH₃)₂)—, —(CH(CH₂CH₃))—, —(CH(CH₃)—CH₂)—, —(CH₂—CH(CH₃))—, —(CH₂—CH₂—CH₂—CH₂)—, —(CH₂—CH₂—CH(CH₃))—, —(CH(CH₃)—CH₂—CH₂)—, —(CH₂—CH(CH₃)—CH₂)—, —(CH₂—C(CH₃)₂)—, —(C(CH₃)₂—CH₂)—, —(CH(CH₃)—CH(CH₃))—, —(CH₂—CH(CH₂CH₃))—, —(CH(CH₂CH₃)—CH₂)—, —(CH(CH₂CH₂CH₃))—, —(CH(CH(CH₃))₂)—and —C(CH₃)(CH₂CH₃)—.

Other examples of alkylene are methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene, hexylene etc.

By the generic terms propylene, butylene, pentylene, hexylene etc. without any further definition are meant all the conceivable isomeric forms with the corresponding number of carbon atoms, i.e. propylene includes 1-methylethylene and butylene includes 1-methylpropylene, 2-methylpropylene, 1,1-dimethylethylene and 1,2-dimethylethylene.

The above definition for alkylene also applies if alkylene is part of another (combined) group such as for example in HO-C_{x-y}alkyleneamino or H₂N-C_{x-y}alkyleneoxy.

Unlike alkyl, alkenyl consists of at least two carbon atoms, wherein at least two adjacent carbon atoms are joined together by a C—C double bond and a carbon atom can only be part of one C—C double bond. If in an alkyl as hereinbefore defined having at least two carbon atoms, two hydrogen atoms on adjacent carbon atoms are formally removed and the free valencies are saturated to form a second bond, the corresponding alkenyl is formed.

Examples of alkenyl are vinyl (ethenyl), prop-1-enyl, allyl (prop-2-enyl), isopropenyl, but-1-enyl, but-2-enyl, but-3-enyl, 2-methyl-prop-2-enyl, 2-methyl-prop-1-enyl, 1-methyl-prop-2-enyl, 1-methyl-prop-1-enyl, 1-methylidenepropyl, pent-1-enyl, pent-2-enyl, pent-3-enyl, pent-4-enyl, 3-methyl-but-3-enyl, 3-methyl-but-2-enyl, 3-methyl-but-1-enyl, hex-1-enyl, hex-2-enyl, hex-3-enyl, hex-4-enyl, hex-5-enyl, 2,3-dimethyl-but-3-enyl, 2,3-dimethyl-but-2-enyl, 2-methylidene-3-methylbutyl, 2,3-dimethyl-but-1-enyl, hexa-1,3-dienyl, hexa-1,4-dienyl, penta-1,4-dienyl, penta-1,3-dienyl, buta-1,3-dienyl, 2,3-dimethylbuta-1,3-diene etc.

By the generic terms propenyl, butenyl, pentenyl, hexenyl, butadienyl, pentadienyl, hexadienyl, heptadienyl, octadienyl, nonadienyl, decadienyl etc. without any further definition are meant all the conceivable isomeric forms with the corresponding number of carbon atoms, i.e. propenyl includes prop-1-enyl and prop-2-enyl, butenyl includes but-1-enyl, but-2-enyl, but-3-enyl, 1-methyl-prop-1-enyl, 1-methyl-prop-2-enyl etc.

Alkenyl may optionally be present in the cis or trans or E or Z orientation with regard to the double bond(s).

The above definition for alkenyl also applies when alkenyl is part of another (combined) group such as for example in C_{x-y}alkenylamino or C_{x-y}alkenyloxy.

Unlike alkyl, alkynyl consists of at least two carbon atoms, wherein at least two adjacent carbon atoms are joined together by a C—C triple bond. If in an alkyl as hereinbefore defined having at least two carbon atoms, two hydrogen atoms in each case at adjacent carbon atoms are formally removed and the free valencies are saturated to form two further bonds, the corresponding alkynyl is formed.

Examples of alkynyl are ethynyl, prop-1-ynyl, prop-2-ynyl, but-1-ynyl, but-2-ynyl, but-3-ynyl, 1-methyl-prop-2-ynyl, pent-1-ynyl, pent-2-ynyl, pent-3-ynyl, pent-4-ynyl, 3-methyl-but-1-ynyl, hex-1-ynyl, hex-2-ynyl, hex-3-ynyl, hex-4-ynyl, hex-5-ynyl etc.

By the generic terms propynyl, butynyl, pentynyl, hexynyl, heptynyl, octynyl, nonynyl, decynyl etc. without any further definition are meant all the conceivable isomeric forms with the corresponding number of carbon atoms, i.e. propynyl includes prop-1-ynyl and prop-2-ynyl, butynyl includes but-1-ynyl, but-2-ynyl, but-3-ynyl, 1-methyl-prop-1-ynyl, 1-methyl-prop-2-ynyl, etc.

If a hydrocarbon chain carries both at least one double bond and also at least one triple bond, by definition it belongs to the alkynyl subgroup.

The above definition for alkynyl also applies if alkynyl is part of another (combined) group, as for example in C_{x-y}alkynylamino or C_{x-y}alkynyloxy.

By heteroatoms are meant oxygen, nitrogen and sulphur atoms.

Haloalkyl (haloalkenyl, haloalkynyl) is derived from the previously defined alkyl (alkenyl, alkynyl) by replacing one or more hydrogen atoms of the hydrocarbon chain independently of one another by halogen atoms, which may be identical or different. If a haloalkyl (haloalkenyl, haloalkynyl) is to be further substituted, the substitutions may take place independently of one another, in the form of mono- or polysubstitutions in each case, on all the hydrogen-carrying carbon atoms.

Examples of haloalkyl (haloalkenyl, haloalkynyl) are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —C≡C—CF₃, —CHFCH₂CH₃, —CHFCH₂CF₃ etc.

From the previously defined haloalkyl (haloalkenyl, haloalkynyl) are also derived the terms haloalkylene (haloalkenylene, haloalkynylene). Haloalkylene (haloalkenylene, haloalkynylene), unlike haloalkyl (haloalkenyl, haloalkynyl), is bivalent and requires two binding partners. Formally, the second valency is formed by removing a hydrogen atom from a haloalkyl (haloalkenyl, haloalkynyl).

Corresponding groups are for example —CH₂F and —CHF—, —CHFCH₂F and —CHFCHF— or >CFCH₂F etc.

The above definitions also apply if the corresponding halogen-containing groups are part of another (combined) group.

Halogen denotes fluorine, chlorine, bromine and/or iodine atoms.

Cycloalkyl is made up of the subgroups monocyclic cycloalkyl, bicyclic cycloalkyl and spiro-cycloalkyl. The ring systems are saturated and formed by linked carbon atoms. In bicyclic cycloalkyl two rings are joined together so that they have at least two carbon atoms in common Forming either fused or bridged ring systems. In spiro-cycloalkyl one carbon atom (spiroatom) belongs to two rings together.

If a cycloalkyl is to be substituted, the substitutions may take place independently of one another, in the form of mono- or polysubstitutions in each case, on all the hydrogen-carrying carbon atoms. Cycloalkyl itself may be linked as a substituent to the molecule via every suitable position of the ring system.

Examples of cycloalkyl are cyclopropyl, cyclobutyl, cyclopentyl, cyclohexyl, cycloheptyl, bicyclo[2.2.0]hexyl, bicyclo[3.2.0]heptyl, bicyclo[3.2.1]octyl, bicyclo[2.2.2]octyl, bicyclo[4.3.0]nonyl (octahydroindenyl), bicyclo[4.4.0]decyl (decahydronaphthyl), bicyclo[2.2.1]heptyl (norbornyl), bicyclo[4.1.0]heptyl (norcaranyl), bicyclo[3.1.1]heptyl (pinanyl), spiro[2.5]octyl, spiro[3.3]heptyl etc.

The above definition for cycloalkyl also applies if cycloalkyl is part of another (combined) group as for example in C_{x-y}cycloalkylamino, C_{x-y}cycloalkyloxy or C_{x-y}cycloalkylalkyl.

If the free valency of a cycloalkyl is saturated, then an alicycle is obtained.

Aryl denotes mono-, bi- or tricyclic carbocycles with at least one aromatic carbocycle.

Preferably, it denotes a monocyclic group with six carbon atoms (phenyl) or a bicyclic group with nine or ten carbon atoms (two six-membered rings or one six-membered ring with a five-membered ring), wherein the second ring may also be aromatic or, however, may also be partially saturated.

If an aryl is to be substituted, the substitutions may take place independently of one another, in the form of mono- or polysubstitutions in each case, on all the hydrogen-carrying carbon atoms. Aryl itself may be linked as a substituent to the molecule via every suitable position of the ring system.

Examples of aryl are phenyl, naphthyl, indanyl (2,3-dihydroindenyl), indenyl, anthracenyl, phenanthrenyl, tetrahydronaphthyl (1,2,3,4-tetrahydronaphthyl, tetralinyl), dihydronaphthyl (1,2-dihydronaphthyl), fluorenyl etc. Most preferred is phenyl.

The above definition of aryl also applies if aryl is part of another (combined) group as for example in arylamino, aryloxy or arylalkyl.

If the free valency of an aryl is saturated, then an arene is obtained.

Heterocyclyl denotes ring systems, which are derived from the previously defined cycloalkyl, cycloalkenyl and aryl by replacing one or more of the groups —CH₂— independently of one another in the hydrocarbon rings by the groups —O—, —S— or —NH— or by replacing one or more of the groups ═CH— by the group ═N—, wherein a total of not more than five heteroatoms may be present, at least one carbon atom must be present between two oxygen atoms and between two sulphur atoms or between an oxygen and a sulphur atom and the ring as a whole must have chemical stability. Heteroatoms may optionally be present in all the possible oxidation stages (sulphur 4 sulfoxide —SO—, sulphone —SO₂—; nitrogen 4 N-oxide). In a heterocyclyl there is no heteroaromatic ring, i.e. no heteroatom is part of an aromatic system.

A direct result of the derivation from cycloalkyl, cycloalkenyl and aryl is that heterocyclyl is made up of the subgroups monocyclic heterocyclyl, bicyclic heterocyclyl, tricyclic heterocyclyl and spiro-heterocyclyl, which may be present in saturated or unsaturated form.

By unsaturated is meant that there is at least one double bond in the ring system in question, but no heteroaromatic system is formed. In bicyclic heterocyclyl two rings are linked together so that they have at least two (hetero)atoms in common forming either fused or bridged ring systems. In spiro-heterocyclyl one carbon atom (spiroatom) belongs to two rings together.

If a heterocyclyl is substituted, the substitutions may take place independently of one another, in the form of mono- or polysubstitutions in each case, on all the hydrogen-carrying carbon and/or nitrogen atoms. Heterocyclyl itself may be linked as a substituent to the molecule via every suitable position of the ring system. Substituents on heterocyclyl do not count for the number of members of a heterocyclyl.

Examples of heterocyclyl are tetrahydrofuryl, pyrrolidinyl, pyrrolinyl, imidazolidinyl, thiazolidinyl, imidazolinyl, pyrazolidinyl, pyrazolinyl, piperidinyl, piperazinyl, oxiranyl, aziridinyl, azetidinyl, 1,4-dioxanyl, azepanyl, diazepanyl, morpholinyl, thiomorpholinyl, homomorpholinyl, homopiperidinyl, homopiperazinyl, homothiomorpholinyl, thiomorpholinyl-S-oxide, thiomorpholinyl-S,S-dioxide, 1,3-dioxolanyl, tetrahydropyranyl, tetrahydrothiopyranyl, [1,4]-oxazepanyl, tetrahydrothienyl, homothiomorpholinyl-S,S-dioxide, oxazolidinonyl, dihydropyrazolyl, dihydropyrrolyl, dihydropyrazinyl, dihydropyridyl, dihydro-pyrimidinyl, dihydrofuryl, dihydropyranyl, tetrahydrothienyl-S-oxide, tetrahydrothienyl-S,S-dioxide, homothiomorpholinyl-S-oxide, 2,3-dihydroazet, 2H-pyrrolyl, 4H-pyranyl, 1,4-dihydropyridinyl, 8-aza-bicyclo[3.2.1]octyl, 8-aza-bicyclo[5.1.0]octyl, 2-oxa-5-azabicyclo[2.2.1]heptyl, 8-oxa-3-aza-bicyclo[3.2.1]octyl, 3,8-diaza-bicyclo[3.2.1]octyl, 2,5-diaza-bicyclo[2.2.1]heptyl, 1-aza-bicyclo[2.2.2]octyl, 3,8-diaza-bicyclo[3.2.1]octyl, 3,9-diaza-bicyclo[4.2.1]nonyl, 2,6-diaza-bicyclo[3.2.2]nonyl, 1,4-dioxa-spiro[4.5]decyl, 1-oxa-3,8-diaza-spiro[4.5]decyl, 2,6-diaza-spiro[3.3]heptyl, 2,7-diaza-spiro[4.4]nonyl, 2,6-diaza-spiro[3.4]octyl, 3,9-diaza-spiro[5.5]undecyl, 2,8-diaza-spiro[4,5]decyl etc.

Further examples are the structures illustrated below, which may be attached via each hydrogen-carrying atom (exchanged for hydrogen):

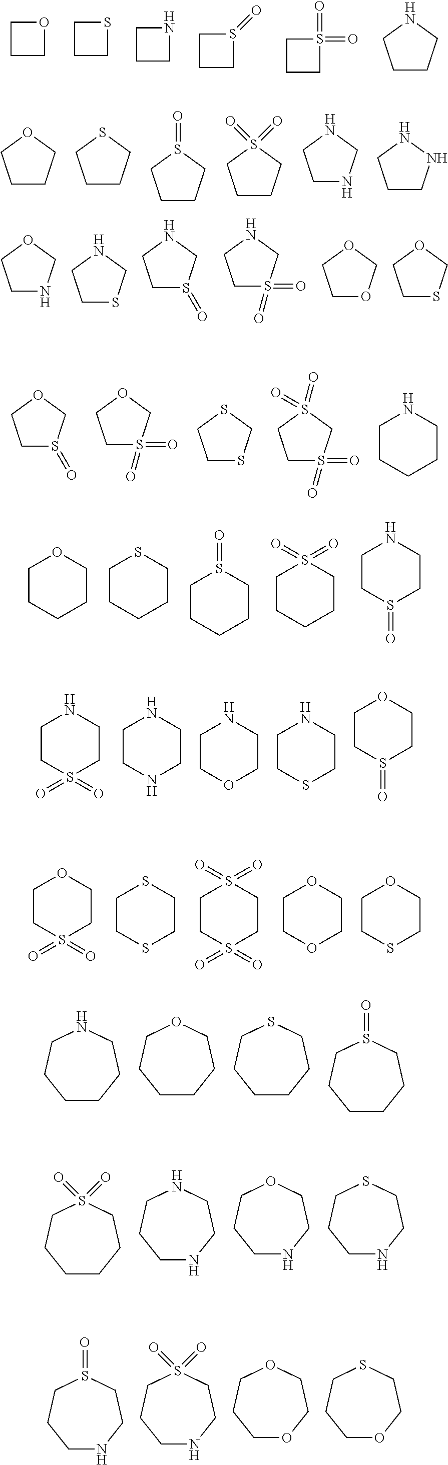

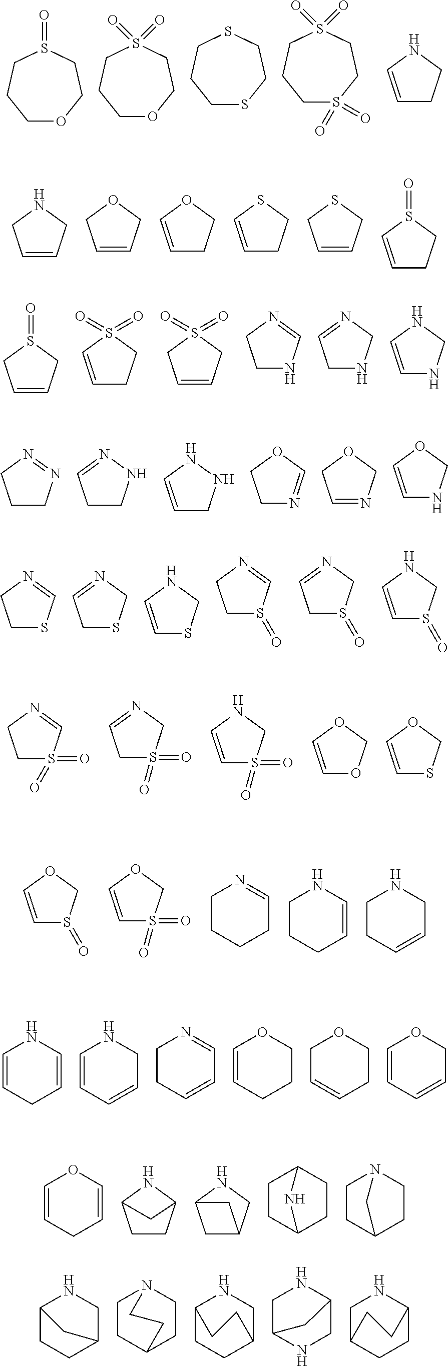

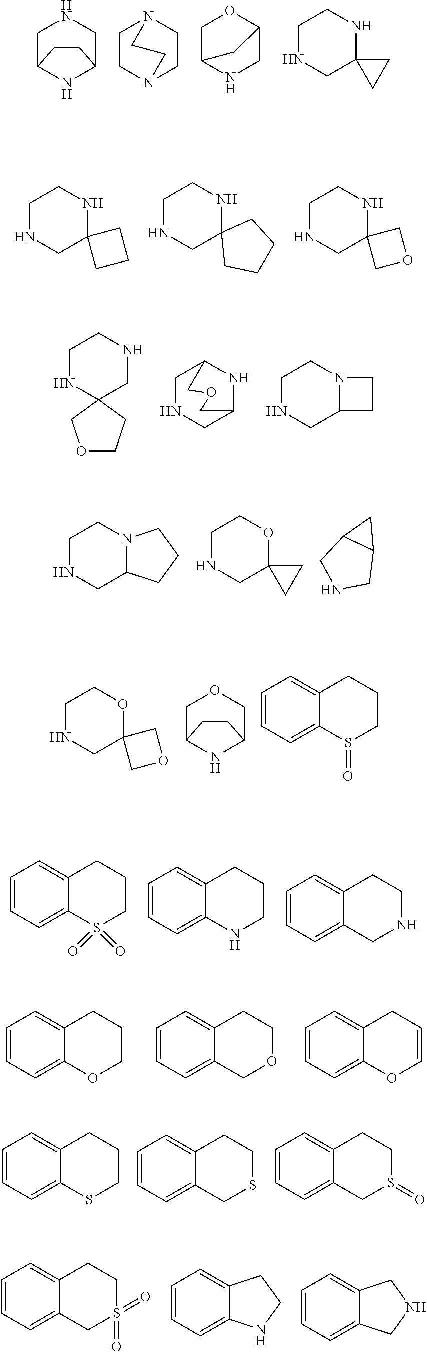

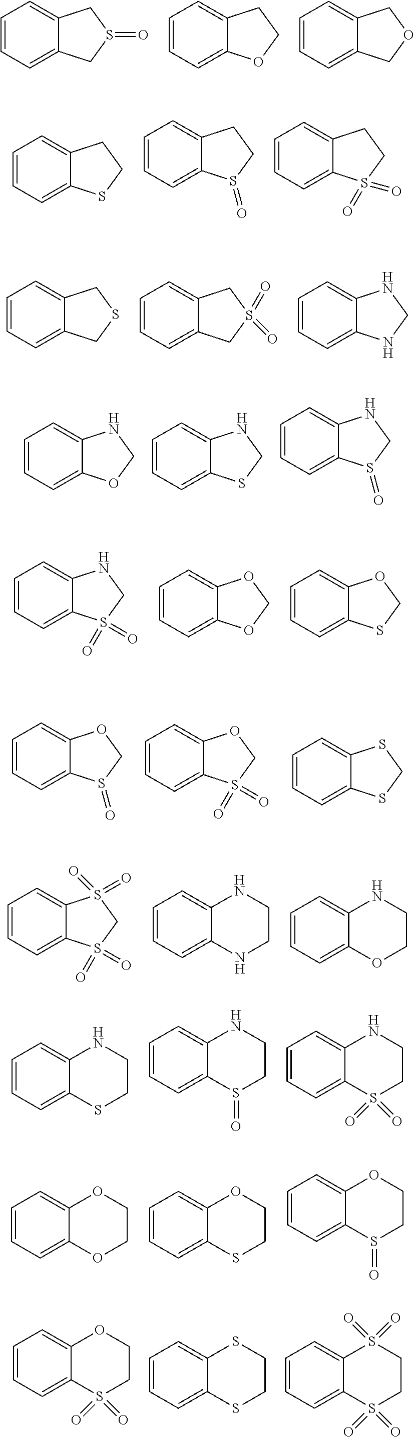

Preferred monocyclic heterocyclyl is 4 to 7 membered and has one or two heteroatoms independently selected from oxygen, nitrogen and sulfur.

Preferred monocyclic heterocyclyls are: piperazinyl, piperidinyl, morpholinyl, pyrrolidinyl, and azetidinyl.

Preferred bicyclic heterocyclyl is 6 to 10 membered and has one or two heteroatoms independently selected from oxygen, nitrogen and sulfur.

Preferred tricyclic heterocyclyl is 9 membered and has one or two heteroatoms independently selected from oxygen, nitrogen and sulfur.

Preferred spiro-heterocyclyl is 7 to 11 membered and has one or two heteroatoms independently selected from oxygen, nitrogen and sulfur.

The above definition of heterocyclyl also applies if heterocyclyl is part of another (combined) group as for example in heterocyclylamino, heterocyclyloxy or heterocyclylalkyl.

If the free valency of a heterocyclyl is saturated, then a heterocycle is obtained.

Heteroaryl denotes monocyclic heteroaromatic rings or polycyclic rings with at least one heteroaromatic ring, which compared with the corresponding aryl or cycloalkyl (cycloalkenyl) contain, instead of one or more carbon atoms, one or more identical or different heteroatoms, selected independently of one another from among nitrogen, sulphur and oxygen, wherein the resulting group must be chemically stable. The prerequisite for the presence of heteroaryl is a heteroatom and a heteroaromatic system.

If a heteroaryl is to be substituted, the substitutions may take place independently of one another, in the form of mono- or polysubstitutions in each case, on all the hydrogen-carrying carbon and/or nitrogen atoms. Heteroaryl itself may be linked as a substituent to the molecule via every suitable position of the ring system, both carbon and nitrogen. Substituents on heteroaryl do not count for the number of members of a heteroaryl.

Examples of heteroaryl are furyl, thienyl, pyrrolyl, oxazolyl, thiazolyl, isoxazolyl, isothiazolyl, pyrazolyl, imidazolyl, triazolyl, tetrazolyl, oxadiazolyl, thiadiazolyl, pyridyl, pyrimidyl, pyridazinyl, pyrazinyl, triazinyl, pyridyl-N-oxide, pyrrolyl-N-oxide, pyrimidinyl-N-oxide, pyridazinyl-N-oxide, pyrazinyl-N-oxide, imidazolyl-N-oxide, isoxazolyl-N-oxide, oxazolyl-N-oxide, thiazolyl-N-oxide, oxadiazolyl-N-oxide, thiadiazolyl-N-oxide, triazolyl-N-oxide, tetrazolyl-N-oxide, indolyl, isoindolyl, benzofuryl, benzothienyl, benzoxazolyl, benzothiazolyl, benzisoxazolyl, benzisothiazolyl, benzimidazolyl, indazolyl, isoquinolinyl, quinolinyl, quinoxalinyl, cinnolinyl, phthalazinyl, quinazolinyl, benzotriazinyl, indolizinyl, oxazolopyridyl, imidazopyridyl, naphthyridinyl, benzoxazolyl, pyridopyridyl, pyrimidopyridyl, purinyl, pteridinyl, benzothiazolyl, imidazopyridyl, imidazothiazolyl, quinolinyl-N-oxide, indolyl-N-oxide, isoquinolyl-N-oxide, quinazolinyl-N-oxide, quinoxalinyl-N-oxide, phthalazinyl-N-oxide, indolizinyl-N-oxide, indazolyl-N-oxide, benzothiazolyl-N-oxide, benzimidazolyl-N-oxide etc.

Further examples are the structures illustrated below, which may be attached via each hydrogen-carrying atom (exchanged for hydrogen):

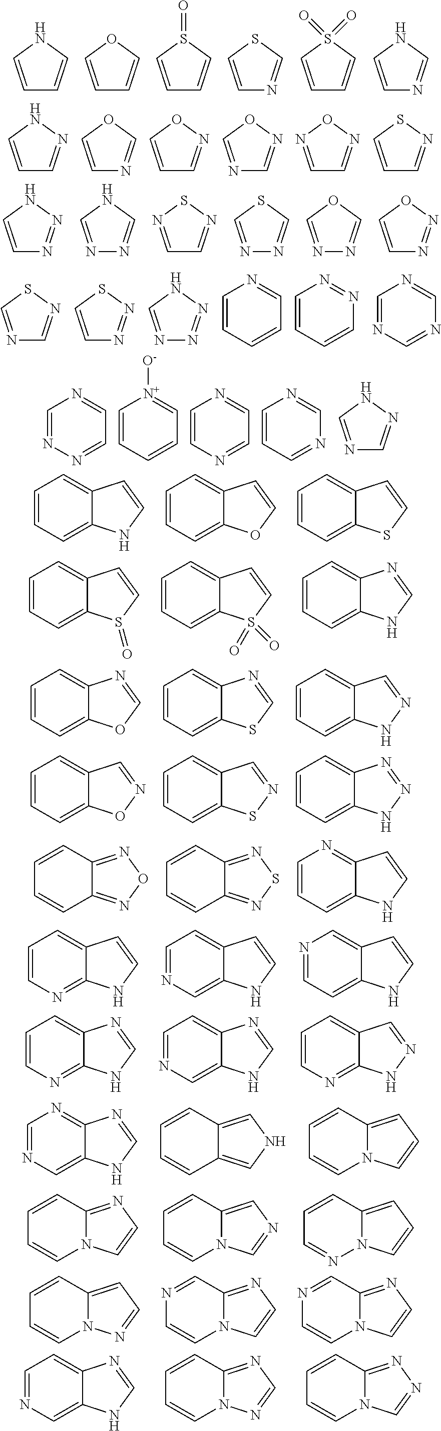

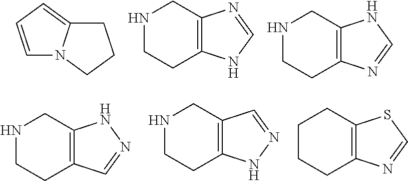

Preferably, heteroaryls are 5-6 membered monocyclic or 9-10 membered bicyclic, each with 1 to 4 heteroatoms independently selected from oxygen, nitrogen and sulfur.

The above definition of heteroaryl also applies if heteroaryl is part of another (combined) group as for example in heteroarylamino, heteroaryloxy or heteroarylalkyl.

If the free valency of a heteroaryl is saturated, a heteroarene is obtained.

By substituted is meant that a hydrogen atom which is bound directly to the atom under consideration, is replaced by another atom or another group of atoms (substituent). Depending on the starting conditions (number of hydrogen atoms) mono- or polysubstitution may take place on one atom. Substitution with a particular substituent is only possible if the permitted valencies of the substituent and of the atom that is to be substituted correspond to one another and the substitution leads to a stable compound (i.e. to a compound which is not converted spontaneously, e.g. by rearrangement, cyclisation or elimination).

Bivalent substituents such as ═S, ═NR, ═NOR, ═NNRR, ═NN(R)C(O)NRR, ═N₂ or the like, may only be substituents on carbon atoms, whereas the bivalent substituents ═O and ═NR may also be a substituent on sulphur. Generally, substitution may be carried out by a bivalent substituent only at ring systems and requires replacement of two geminal hydrogen atoms, i.e. hydrogen atoms that are bound to the same carbon atom that is saturated prior to the substitution. Substitution by a bivalent substituent is therefore only possible at the group —CH₂— or sulphur atoms (═O group or ═NR group only, one or two ═O groups possible or, e.g., one ═O group and one ═NR group, each group replacing a free electron pair) of a ring system.

Isotopes: It is to be understood that all disclosures of an atom or compound of the invention include all suitable isotopic variations. In particular, a reference to hydrogen also includes deuterium.

Stereochemistry/solvates/hydrates: Unless specifically indicated, throughout the specification and appended claims, a given chemical formula or name shall encompass tautomers and all stereo, optical and geometrical isomers (e.g. enantiomers, diastereomers, E/Z isomers, etc.) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases, or by resolution of a racemic mixture using an appropriate resolving agent, e.g. by means of diastereomeric salt formation of the racemic compound with optically active acids or bases, subsequent resolution of the salts and release of the desired compound from the salt, or by derivatization of the corresponding racemic compounds with optically active chiral auxiliary reagents, subsequent diastereomer separation and removal of the chiral auxiliary group, or by kinetic resolution of a racemate (e.g. by enzymatic resolution); by enantioselective crystallization from a conglomerate of enantiomorphous crystals under suitable conditions, or by (fractional) crystallization from a suitable solvent in the presence of an optically active chiral auxiliary.

Salts: The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication and commensurate with a reasonable benefit/risk ratio.

As used herein “pharmaceutically acceptable salts” refers to derivatives of the disclosed compounds wherein the parent compound is modified by making acid or base salts thereof.

Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts), also comprise a part of the invention.

In a representation such as for example

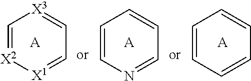

the letter A has the function of a ring designation in order to make it easier, for example, to indicate the attachment of the ring in question to other rings.

For bivalent groups in which it is crucial to determine which adjacent groups they bind and with which valency, the corresponding binding partners are indicated in brackets where necessary for clarification purposes, as in the following representations:

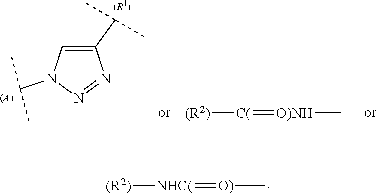

If such a clarification is missing then the bivalent group can bind in both directions, i.e., e.g., —C(═O)NH— also includes —NHC(═O)— (and vice versa).

Groups or substituents are frequently selected from among a number of alternative groups/substituents with a corresponding group designation (e.g. R^{a}, R^{b} etc). If such a group is used repeatedly to define a compound according to the invention in different parts of the molecule, it is pointed out that the various uses are to be regarded as totally independent of one another.

By a therapeutically effective amount for the purposes of this invention is meant a quantity of substance that is capable of obviating symptoms of illness or of preventing or alleviating these symptoms, or which prolong the survival of a treated patient.

List of abbreviations

| | |
|---|---|
| Ac | acetyl |
| ACN | acetonitrile |
| aq. | aquatic, aqueous |
| ATP | adenosine triphosphate |
| Bn | benzyl |
| Boc | tert-butyloxycarbonyl |
| Bu | butyl |
| c | concentration |
| CDI | 1,1′-carbonyldiimidazole |
| d | day(s) |
| DBU | Diazabicycloundecen |
| DCM | dichloromethane |
| DEA | Diethylamine |
| DIPEA | N-ethyl-N,N-diisopropylamine (Hunig's base) |
| DMAP | 4-N,N-dimethylaminopyridine |
| DME | 1,2-dimethoxyethane |
| DMF | N,N-dimethylformamide |
| DMSO | Dimethyl sulfoxide |
| dppf | 1.1′-bis(diphenylphosphino)ferrocene |
| equiv. | equivalent(s) |
| ESI | electron spray ionization |
| Et | ethyl |
| Et₂O | diethyl ether |
| EtOAc | ethyl acetate |
| EtOH | ethanol |
| h | hour(s) |
| HATU | O-(7-azabenzotriazol-1-yl)-N,N,N′,N′-tetramethyl-uronium |
| | hexafluorophosphate |
| HPLC | high performance liquid chromatography |
| i | iso |
| conc. | concentrated |
| LC | liquid chromatography |
| LiHMDS | lithium bis(trimethylsilyl)amide |
| m-CPBA | meta-chloroperoxybenzoic acid |
| Me | methyl |
| MeOH | methanol |
| min | minute(s) |
| MS | mass spectrometry |
| NP | normal phase |
| n.a. | not available |
| PBS | phosphate-buffered saline |
| Ph | phenyl |
| Pr | propyl |
| Py | pyridine |
| rac | racemic |
| red. | reduction |
| Rf (R_{f}) | retention factor |
| RP | reversed phase |
| rt | ambient temperature |
| s | second(s) |
| sat. | saturated |
| SFC | supercritical fluid chromatography |
| S_{N} | nucleophilic substitution |
| tBu | tert-butyl |
| TEA | triethyl amine |
| temp. | temperature |
| tert | tertiary |
| Tf | triflate |
| TFA | trifluoroacetic acid |
| THF | tetrahydrofuran |
| TLC | thin layer chromatography |
| t_{Ret.} | retention time (HPLC) |
| Ts | tosylate |
| UPLC | ultra performance liquid chromatography |
| UV | ultraviolet |

### EXAMPLES

Features and advantages of the present invention will become apparent from the following detailed examples which illustrate the principles of the invention by way of example without restricting its scope:

### Preparation of the Compounds According to the Invention

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

If a compound is to be represented both by a structural formula and by its nomenclature, in the event of a conflict the structural formula is decisive.

Microwave reactions are carried out in an initiator/reactor made by Biotage or in an Explorer made by CEM or in Synthos 3000 or Monowave 3000 made by Anton Paar in sealed containers (preferably 2, 5 or 20 mL), preferably with stirring.

### Chromatography

The thin layer chromatography is carried out on ready-made silica gel 60 TLC plates on glass (with fluorescence indicator F-254) made by Merck.

The preparative high pressure chromatography (RP HPLC) of the example compounds according to the invention is carried out on Agilent or Gilson systems with columns made by Waters (names: SunFire™ Prep C18, OBD™ 10 μm, 50×150 mm or SunFire™ Prep C18 OBD™ 5 μm, 30×50 mm or XBridge™ Prep C18, OBD™ 10 μm, 50×150 mm or XBridge™ Prep C18, OBD™ 5 μm, 30×150 mm or XBridge™ Prep C18, OBD™ 5 μm, 30×50 mm) and YMC (names: Actus-Triart Prep C18, 5 μm, 30×50 mm).

Different gradients of H₂O/ACN are used to elute the compounds, while for Agilent systems 5% acidic modifier (20 mL HCOOH to 1 L H₂O/ACN (1/1)) is added to the water (acidic conditions). For Gilson systems to water is added 0.1% HCOOH.

For the chromatography under basic conditions for Agilent systems H₂O/ACN gradients are used as well, while the water is made alkaline by addition of 5% basic modifier (50 g NH₄HCO₃+50 mL NH₃ (25% in H₂O) to 1 L with H₂O). For Gilson systems the water is made alkaline as follows: 5 mL NH₄HCO₃ solution (158 g in 1 L H₂O) and 2 mL NH₃ (28% in H₂O) are replenished to 1 L with H₂O.

The supercritical fluid chromatography (SFC) of the intermediates and example compounds according to the invention is carried out on a JASCO SFC-system with the following colums: Chiralcel OJ (250×20 mm, 5 μm), Chiralpak AD (250×20 mm, 5 μm), Chiralpak AS (250×20 mm, 5 μm), Chiralpak IC (250×20 mm, 5 μm), Chiralpak IA (250×20 mm, 5 μm), Chiralcel OJ (250×20 mm, 5 μm), Chiralcel OD (250×20 mm, 5 μm), Phenomenex Lux C2 (250×20 mm, 5 μm).

The analytical HPLC (reaction control) of intermediate and final compounds is carried out using columns made by Waters (names: XBridge™ C18, 2.5 μm, 2.1×20 mm or XBridge™ C18, 2.5 μm, 2.1×30 mm or Aquity UPLC BEH C18, 1.7 μm, 2.1×50 mm) and YMC (names: Triart C18, 3.0 μm, 2.0×30 mm) and Phenomenex (names: Luna C18, 5.0 μm, 2.0×30 mm). The analytical equipment is also equipped with a mass detector in each case.

### HPLC-Mass Spectroscopy/UV-Spectrometry

The retention times/MS-ESI⁺ for characterizing the example compounds according to the invention are produced using an HPLC-MS apparatus (high performance liquid chromatography with mass detector). Compounds that elute at the injection peak are given the retention time t_{Ret.}=0.00.

### Method A

| HPLC | Agilent 1200 system | |
|---|---|---|
| MS | 1200Series LC/MSD(API-ES +/− | |
| | 3000 V, Quadrupol, G6140) | |
| MSD signal settings | Scan pos/neg 120-900 m/z | |
| Detection signal | 315 nm (bandwidth 170 nm, reference off) | |
| Spectrum range | 230-400 nm | |
| Peak width | <0.01 min | |
| Column | Waters, Xbridge C18, 2.5 μm, | |
| | 2.1 × 20 mm column | |
| Column temperature | 60° C. | |
| Solvent | A: 20 mM aq. NH₄HCO₃/NH₃ pH 9 | |
| | B: ACN HPLC grade | |
| Flow | 1.00 mL/min | |
| Gradient | 0.00-1.50 min | 10% to 95% B |
| | 1.50-2.00 min | 95% B |
| | 2.00-2.10 min | 95% to 10% B |

### Method B

| HPLC | Agilent 1100/1200/1260 system | |
|---|---|---|
| MS | 1200 Series LC/MSD (MM-ES + | |
| | APCI +/− 3000 V, | |
| | Quadrupol, G6130B) | |
| MSD signal | Scan pos/neg | |
| settings | 50-500 | |
| Column | Waters, Part. No. 186003389, | |
| | XBridge BEH C18, | |
| | 2.5 × 30 mm) column | |
| Solvent | A: 5 mM NH₄HCO₃/ | |
| | 18 mM NH₃ (pH = 9.2) | |
| | B: acetonitrile (HPLC grade) | |
| Detection signal | UV 254 nm, 230 nm, 214 nm | |
| | (bandwidth 8, reference off) | |
| Spectrum | range: 190-400 nm; slit: 4 nm | |
| Peak width | >0.0031 min (0.063 s response time, 80 Hz) | |
| Injection | 0.5 μL standard injection | |
| Flow | 1.4 mL/min | |
| Column temperature | 45° C. | |
| Gradient | 0.0-1.0 min | 15% → 95% B |
| | 1.0-1.1 min | 95% B |
| | Stop time: | |
| | 1.3 min | |

### Method C

| | |
|---|---|
| HPLC | Agilent 1260 system |
| MS | 1200 Series LC/MSD (MM-ES + APCI +/− |
| | 3000 V, Quadrupol, G6130) |
| Detection | UV: 254 nm (bandwidth 8, reference off) |
| | UV: 230 nm (bandwidth 8, reference off) |
| | UV spectrum range: 190-400 nm; step: 4 nm |
| | MS: positive and negative mode |
| Mass range | 100-800 m/z |
| Column | Waters; Part. No. 186003389; |
| | XBridge BEH C18, 2.5 μm, |
| | 30 × 2.1 mm |
| Column temperature | 45° C. |
| Solvent | A: 5 mM NH4HCO3/19 mM NH3 in |
| | H2O; B: ACN (HPLC grade) |
| Flow | 1.40 mL/min |
| Gradient | 0.00-1.00 min: 5% B to 100% B |
| | 1.00-1.37 min: 100% B |
| | 1.37-1.40 min: 100% B to 5% B |

### Method D

| LC-MS | Waters Arc-HPLC-SQ Detector-2 | |
|---|---|---|
| MSD signal settings | ESI Scan pos & neg | |
| | Capillary Voltage 3.50 Kv cone | |
| | voltage 30 V Disolvation gas | |
| | 750 L/hr Disolvation Temp 350° C. | |
| Column | X-Bridge C18, 4.6 × 75 mm, 3.5 μm | |
| Column temperature | 40° C. | |
| Solvent | A: 10 mM Ammonium Acetate in water | |
| | B: ACN | |
| Flow | 1.2 mL/min | |
| Gradient | 0.0-0.3 min | 2% B |
| | 0.3-1.3 min | 2% to 98% B |
| | 1.3-4.5 min | 98% B |
| | 3.0-4.8 min | 100% B |

### Method F

| HPLC | Agilent 1100/1200 system | |
|---|---|---|
| MS | 1200 Series LC/MSD (API-ES +/− | |
| | 3000/3500 V, Quadrupol, G6140A) | |
| MSD signal settings | Scan pos/neg 150-750 | |
| Detection signal | UV 254 nm, 230 nm, 214 nm | |
| | (bandwidth 10, reference off) | |
| Spectrum | range: 190-400 nm; slit: 4 nm | |
| Peak width | >0.0031 min (0.063 s response time, 80 Hz) | |
| Column | YMC; Part. No. TA12S03-0302WT; Triart | |
| | C18, 3 μm, 12 nm; 30 × 2.0 mm column | |
| Column temperature | 45° C. | |
| Solvent | A: H2O + 0.11% formic acid | |
| | B: ACN + 0.1% formic acid (HPLC grade) | |
| Flow | 1.4 mL/min | |
| Gradient | 0.0-1.0 min | 15% to 95% B |
| | 1.0-1.1 min | 95% B |

| | |
|---|---|
| Stop time: | 1.23 min |

### SFC

### Method SFC-1

| Make | Waters UPC²-MS | | |
|---|---|---|---|
| Soft | Empower3 | | |
| MS | QDa | | |
| Column | CHIRALCEL OX-3(4.6*150 MM) 3 μm | | |
| A-Solvent | CO₂ | | |
| B-solvent | ACN | | |
| Total Flow | 3 g/min | | |
| % of Co-Solvent | 15 | | |
| ABPR | 1500 psi | | |
| Colum temp | 30° C. | | |
| PDA range | 200 nm to 400 nm | | |
| Resolution | 1.2 nm | | |
| MS Parameters | — | | |
| QDa MS scan range | 100 Da to 1000 Da | | |
| | Cone voltage | | |
| Positive scan | 20 V | | |
| Negative Scan | 15 V | | |
| | 12 min | 1% B | 2 mL/min |

| | |
|---|---|
| Pressure limit | 20-550 bar |

### Method SFC-2

| | |
|---|---|
| Make | Waters UPC²-MS |
| Column | CHIRALPAK AD H(4.6*250 MM) 5 μm |
| A-Solvent | CO₂ |
| B-solvent | 0.5% Methanolic Ammonia in Methanol |
| Total Flow | 3 mL/min |
| % of Co-Solvent | 10 |
| ABPR | 1500 psi |
| Colum temp | 30° C. |
| PDA range | 200 nm to 400 nm |
| MS Parameters | — |

### Method X

| UPLC-MS | Waters Acquity-Binary Solvent | |
|---|---|---|
| | Manager-UPLC-SQ Detector-2 | |
| MSD signal settings | Scan pos & neg 100-1500, | |
| | Source Voltage: Capillary | |
| | Vol(kV)- 3.50, Cone(V): 50 | |
| | Source Temp: Desolvation Temp(° C.): 350 | |
| | Source Gas Flow: Desolvation(L/Hr): | |
| | 750, Cone (L/hr): 50 | |
| Column | XBridge BEH C18 2.5 μm, 2.1 × 50 mm | |
| Eluent | A: 0.07% formic acid in Acetonitrile | |
| | B: 0.07% formic acid in water | |
| Detection signal | Diode Array | |
| Spectrum | Range: 200-400 nm; Resolution: 1.2 nm | |
| Sampling rate | 20 point/sec | |
| Injection | 0.5 μL standard injection | |
| Flow | 0.7 mL/min | |
| Column temperature | 35° C. | |
| Gradient | 0.0-0.40 min | 95% B |
| | 0.40-1.0 min | 95% → 5% B |
| | 1.00-2.00 min | 5% B |
| | 2.00-2.10 min | 5% → 95% B |
| | 2.10-2.50 min | 95% B |

### Method Y

| UPLC-MS | Waters Acquity-UPLC-SQ Detector-2 | |
|---|---|---|
| MSD signal settings | Scan pos & neg 100-1500, | |
| | Source Voltage: Capillary Vol(kV)- | |
| | 3.50, Cone(V): 50 | |
| | Source Temp: Desolvation Temp(° C.): 350 | |
| | Source Gas Flow: Desolvation(L/Hr): | |
| | 700, Cone (L/hr): 50 | |
| Column | AQUITY UPLC BEH C18 1.7 μm, 2.1 × 50 mm | |
| Eluent | A: 0.07% formic acid in Acetonitrile | |
| | B: 0.07% formic acid in water | |
| Detection signal | Diode Array | |
| Spectrum | Range: 200-400 nm; Resolution: 1.2 nm | |
| Sampling rate | 20 point/sec | |
| Injection | 0.5 μL standard injection | |
| Flow | 0.6 mL/min | |
| Column temperature | 35° C. | |
| Gradient | 0.0-0.40 min | 97% B |
| | 0.40-2.50 min | 97% → 2% B |
| | 2.50-3.40 min | 2% B |
| | 3.40-3.50 min | 2% → 97% B |
| | 3.50-4.00 min | 97% B |

| | |
|---|---|
| ELSD Parameters: - | GAS: 40 psi, Gain: 500, Drift Temp: 45° C. |

### Method AB

| UPLC-MS | SHIMADZU LCMS-2020, LabSolution | |
|---|---|---|
| | Version 5.97SP1 | |
| MSD signal settings | ESI Scan pos 120-1000, Drying Gas: N₂ | |
| | Drying Gas Flow: 15(L/min) | |
| | DL Voltage: 120(v) | |
| | Qarray DC Voltage: 20(V) | |
| Column | Kinetex ® EVO C18 2.1 × 30 mm 5 um | |
| Eluent | A: 0.0375% TFA in water (v/v) | |
| | B: 0.01875% TFA in Acetonitrile (v/v) | |
| Detection signal | PDA Detector | |
| Spectrum | Range: 120-1000 nm | |
| Flow | 2.0 mL/min | |
| Column temperature | 50° C. | |
| Gradient | 0.0-0.6 min | 0% → 60% B |
| | 0.6-0.78 min | 60% |
| | 0.78-0.79 min | 60% → 0% B |
| | 0.79-0.80 min | 0% B |

### Method AC

| HPLC-MS | Waters - Alliance 2695, SQ Detector-2 | |
|---|---|---|
| MSD signal settings | Scan pos & neg 100-1500, | |
| | Source Voltage: Capillary Vol(kV)- | |
| | 3.50, Cone(V): 35 | |
| | Source Temp: Desolvation Temp(° C.): 350 | |
| | Source Gas Flow: Desolvation(L/Hr): 650, | |
| Column | XBridge C18 (4.6 × 75) mm, 3.5 μm | |
| Eluent | A: 10 mM Ammonium Bicarbonate in Water | |
| | B: Acetonitrile | |
| Flow | 1.30 mL/min | |
| Column temperature | 35° C. | |
| Gradient | 0.0-0.50 min | 5% B |
| | 0.50-1.0 min | 5% → 15% B |
| | 1.00-4.00 min | 15% → 98% B |
| | 4.00-7.0 min | 98% B |
| | 7.0-7.50 min | 98% à5% B |
| | 7.50-8.0 min | 5% B |

### Method AD

| UPLC-MS | Waters Acquity-UPLC-SQ Detector-2 | |
|---|---|---|
| MSD signal settings | Scan pos & neg 100-1500, | |
| | Source Voltage: Capillary Vol(Kv)- | |
| | 3.50, Cone(V): 30 | |
| | Source Temp: Desolvation Temp(° C.): 350 | |
| | Source Gas Flow: Desolvation (L/Hr): 700, | |
| Column | Acquity UPLC BEH C18 (3.0 × 30)mm 1.7 μm | |
| Eluent | A: 0.05% formic acid in Water | |
| | B: 0.05% formic acid in ACN | |
| Flow | 0.85 mL/min | |
| Column temperature | 50° C. | |
| Gradient | 0.0-0.05 min | 3% B |
| | 0.05-1.20 min | 3% · 98% B |
| | 1.20-1.75 min | 98% B |
| | 1.75-1.80 min | 98% · 3% B |
| | 1.80-2.10 min | 3% B |

| | |
|---|---|
| ELSD Parameters: | Gas -50 PSI; GAIN-500, DRIFT TEMP-50° C. |

### Method AE

| | |
|---|---|
| Instrument | Waters - UPLC Aquity |
| | Mobile Phase-A: 0.05% Trifluoroacetic |
| | acid in Water |
| | Mobile Phase-B: 0.05% Trifluoroacetic |
| | acid Acetonitrile |
| Gradient Programme: | [Time in min/% of B]: 0/5, 2.0/5, |
| | 6.0/90, 8.5/90, 9.0/5, 10/5 |
| Column: | Aquity-UPLC HSS C18 (3.0 × 150 mm) 1.8 μm |
| Column Temp: | 50° C. |
| Flow rate: | 0.3 mL/min |
| Detector: | PDA Detector |
| Spectrum Range: | 210-400 nm |
| ELSD Parameters: | Nebuliser temp-40° |
| | Evaporator temp-45° |
| | Gas Flow Rate-3.0 |
| ELSD Parameters: | Gas -50 PSI; GAIN-500, DRIFT TEMP-50° C. |

### Method AF

| | |
|---|---|
| Instrument | Waters - Arc-HPLC-SQ Detector-2 |
| | Mobile Phase-A: 10 mM Ammonium Acetate |
| | in water |
| | Mobile Phase-B: Acetonitrile |
| Gradient Programme: | [Time in min/% of B]: 0/2, 0.3/2, |
| | 1.3/98, 4.5/98, 5.0/2, 5.01/2 |
| Column: | X-Bridge C18, 4.6 × 75 mm, 3.5 μm |
| Column Temp: | 40° C. |
| Flow rate: | 1.2 mL/min |
| Detector: | ELSD Detector |
| Spectrum Range: | 210-400 nm |
| ELSD Parameters: | Nebuliser temp-40° |
| | Evaporator temp-45° |
| | Gas Flow Rate-3.0 |

### Method AG

| HPLC-MS | Agilent RRLC, Agilent SQD | |
|---|---|---|
| MSD signal settings | Scan pos & neg 100-1500, | |
| | Source Voltage: Capillary Vol(kV)- | |
| | 3.50, Cone(V): 25 to 50 | |
| | Source Temp: Desolvation Temp(° C.): 350 | |
| | Source Gas Flow: Desolvation(L/Hr): 650, | |
| Column | Atlantis T3 (4.6 × 250) mm, 5 μm | |
| Eluent | A: 10 mM Ammonium Bicarbonate in Water | |
| | B: Acetonitrile | |
| Flow | 1.30 mL/min | |
| Column temperature | 35° C. | |
| Gradient | 0.0-0.50 min | 5% B |
| | 0.50-1.0 min | 5% → 15% B |
| | 1.00-4.00 min | 15% → 98% B |
| | 4.00-7.0 min | 98% B |
| | 7.0-7.50 min | 98% → 5% B |
| | 7.50-8.0 min | 5% B |

### Method AH

| | |
|---|---|
| Instrument | Waters - Agilent 1290 Infinity, Agilent SQD |
| | Mobile Phase-A: 0.1& Formic Acid in Water |
| | Mobile Phase-B: 0.1% Formic Acid in |
| | Acetonitrile |
| Gradient Programme: | [Time in min/% of B]: 0/3, 0.4/3, |
| | 3.2/98, 3.8/98, 4.2/3, 4.5/3 |
| Column: | Aquity BEH C18, 2.1 × 50 mm, 1.7 μm |
| Column Temp: | 25° C. |
| Flow rate: | 0.6 mL/min |
| Detector: | ELSD Detector |
| Spectrum Range: | 210-400 nm |
| ELSD Parameters: | Nebuliser temp-40° |
| | Evaporator temp-45° |
| | Gas Flow Rate-3.0 |

The compounds according to the invention and intermediates are prepared by the methods of synthesis described hereinafter in which the substituents of the general formulae have the meanings given hereinbefore. These methods are intended as an illustration of the invention without restricting its subject matter and the scope of the compounds claimed to these examples. Where the preparation of starting compounds is not described, they are commercially obtainable or their synthesis is described in the prior art or they may be prepared analogously to known prior art compounds or methods described herein, i.e. it is within the skills of an organic chemist to synthesize these compounds. Substances described in the literature can be prepared according to the published methods of synthesis. If a chemical structure in the following is depicted without exact configuration of a stereo center, e.g. of an asymmetrically substituted carbon atom, then both configurations shall be deemed to be included and disclosed in such a representation. The representation of a stereo center in racemic form shall always deem to include and disclose both enantiomers (if no other defined stereo center exists) or all other potential diastereomers and enantiomers (if additional, defined or undefined, stereo centers exist).

### Synthesis of N,N-Bis-Substituted 3-Amino Pyrrolidines

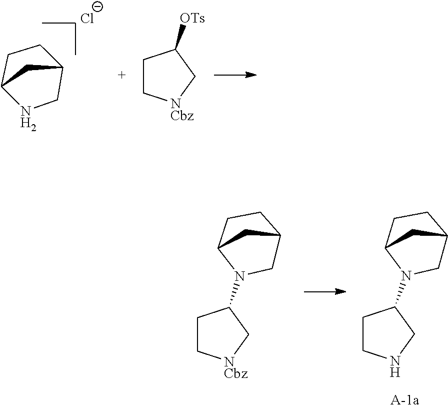

A solution of benzyl (3R)-3-[(4-methylbenzenesulfonyl)oxy]pyrrolidine-1-carboxylate (750 mg, 2.00 mmol, 1.0 equiv.) in acetonitrile (ACN) is added to a mixture of (1R,4S)-2-azabicyclo[2.2.1]heptane hydrochloride (309.1 mg, 2.2 mmol, 1.1 equiv.) and Caesium carbonate (1953 mg, 5.99 mmol, 3.0 equiv.). The reaction mixture is heated to 75° C. and stirred for 3 h.

After cooling to room temperature, the reaction mixture is diluted with CH₂Cl₂ and extracted with water. The aqueous phase is further extracted with DCM, the combined organic phases are dried over magnesium sulfate (MgSO4), filtered and the solvent is removed under reduced pressure. The residue is redissolved in MeOH (5 mL) and subsequently added to a slurry of Pd/C in CH₂Cl₂ (5 mL). The reaction mixture is pressurized with hydrogen gas (5 bar) and stirred at room temperature for 4 h. CH₂Cl_{z} is added and the suspension is filtered over celite. The filtrate is evaporated to dryness and the residue is purified by chromatography to yield product A-1a.

The following intermediate compounds (Table 1) are obtained analogously.

TABLE 1

| | | | | HPLC |
|---|---|---|---|---|
| # | Structure | tᵣₑₜ [min] | [M + H]⁺ | method |
| A-1a | 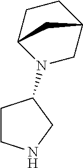 [SMILES: C1C[C@H](N2C[C@H]3CC[C@@H]2C3)CN1] | 0.75 | 167 | A |
| A-1b | 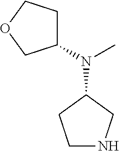 | 0.80 | 171 | A |
| A-1c | 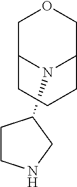 [SMILES: C1CC2COCC(C1)N2[C@H]1CCNC1] | 0.83 | 183 | A |
| A-1d | 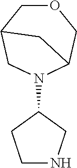 [SMILES: C1C[C@H](N2CC3COCC2C3)CN1] | 0.87 | 183 | A |
| A-1e | 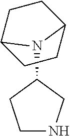 [SMILES: C1C[C@H](N2C3CCC2CC3)CN1] | 0.84 | 203 | A |
| A-1f | 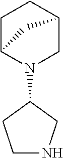 [SMILES: C1C[C@H](N2C[C@@H]3CC[C@H]2C3)CN1] | 0.85 | 167 | A |
| A-1g | 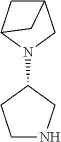 [SMILES: C1C[C@H](N2CC3CC2C3)CN1] | 0.84 | 153 | A |
| A-1h | 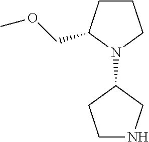 | 0.76 | 185 | A |
| A-1i | 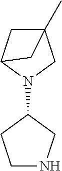 | 0.87 | 167 | A |
| A-1j | 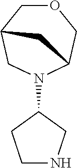 [SMILES: C1C[C@H](N2C[C@@H]3COC[C@H]2C3)CN1] | 0.83 | 183 | A |
| A-1k | 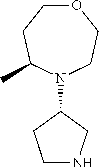 | 0.89 | 184 | A |
| A-11 | 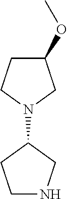 | 0.88 | 170 | A |
| A-1m | 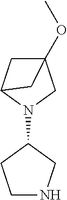 | 0.90 | 182 | A |
| A-1n | 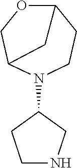 [SMILES: C1C[C@H](N2CCC3CC2CO3)CN1] | 0.89 | 183 | A |
| A-10 | 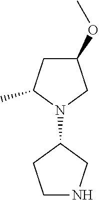 | 0.82 | 185 | A |
| A-1p | 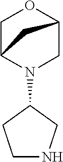 [SMILES: C1C[C@H](N2C[C@@H]3C[C@H]2CO3)CN1] | 0.82 | 169 | A |
| A-1q | 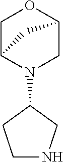 [SMILES: C1C[C@H](N2C[C@H]3C[C@@H]2CO3)CN1] | 0.82 | 169 | A |
| A-1r | 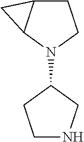 [SMILES: C1C[C@H](N2CCC3CC32)CN1] | 0.90 | 153 | A |
| A-1s | 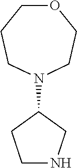 [SMILES: C1COCCN([C@H]2CCNC2)C1] | 0.85 | 170 | A |
| A-1t | 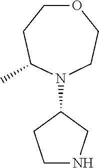 | 0.87 | 184 | A |
| A-1u | 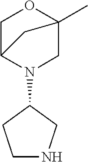 | 0.84 | 182 | A |
| A-1v | 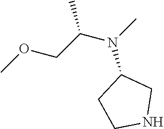 | 0.91 | 172 | A |
| A-1w | 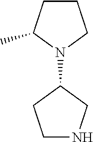 | 0.75 | 191 | A |

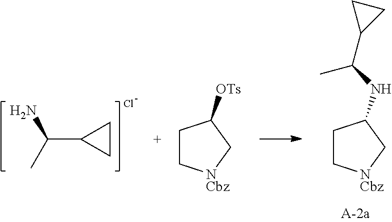

(3R)-3-[(4-methyl benzenesulfonyl)oxy]pyrrolidine-1-carboxylate (500 mg, 1.67 mmol, 1.0 equiv.), (R)-1-cyclopropylethanamine hydrochloride (428 mg 3.34 mmol, 2.0 equiv) and potassium carbonate (923 mg, 6.68 mg, 4.0 equiv.) are dissolved in ACN (5.0 mL). The reaction is stirred for 5 min at room temperature before heating up to 90° C. for 2 days. Upon completion the reaction is extracted with DCM and water. The organic layer is evaporated and purified by NP chromatography to afford A-2a.

The following intermediate compounds (Table 2) are obtained analogously.

TABLE 2

| | | | | HPLC |
|---|---|---|---|---|
| # | Structure | tᵣₑₜ [min] | [M + H]⁺ | method |
| A-2a | 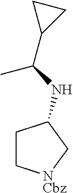 [SMILES: C[C@H](N[C@H]1CCN(C(=O)OCc2ccccc2)C1)C1CC1] | 0.80 | 289 | F |
| A-2b | 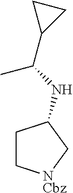 [SMILES: C[C@@H](N[C@H]1CCN(C(=O)OCc2ccccc2)C1)C1CC1] | 0.84 | 289 | F |

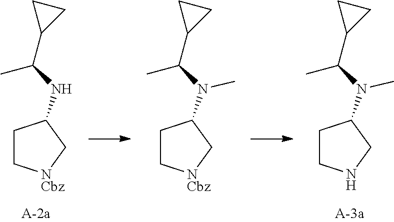 [SMILES: C[C@H](N[C@H]1CCN(C(=O)OCc2ccccc2)C1)C1CC1 ; C[C@@H](C1CC1)N(C)[C@H]1CCN(C(=O)OCc2ccccc2)C1 ; C[C@@H](C1CC1)N(C)[C@H]1CCNC1]

To an ice-cold solution of A-2a (218 mg, 0.76 mmol, 1.0 equiv.) in DMF (2 mL), NaH (51.0 mg, 1.28 mmol, 1.7 equiv.) is added and stirred at room temperature for 10 min. Then, methyl iodide (0.14 mL, 2.24 mmol, 2.96 equiv.) is added and the reaction is stirred at room temperature for 1 h. Upon completion the reaction is quenched with water and extracted with CH₂Cl₂ and sat. NaHCO₃ solution. The organic layer is dried under reduced pressure. The residue is redissolved in CH₂Cl₂:MeOH (1:2) (15 mL) and Pd/C (20 mg) is added. The reaction mixture is pressurized with hydrogen gas (6 bar) and stirred at room temperature for 2.5 h. The reaction mixture is filtered over celite and reduced to dryness under reduced pressure at 50° C. to afford A-3a.

The following intermediate compounds (Table 3) are obtained analogously.

TABLE 3

| | | | | HPLC |
|---|---|---|---|---|
| # | Structure | tᵣₑₜ [min] | [M + H]⁺ | method |
| A-3a | 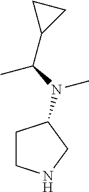 | 0.27 | 169 | F |
| A-3b | 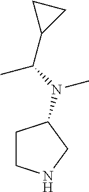 | 0.29 | 169 | F |

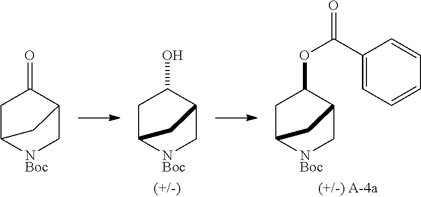 [SMILES: CC(C)(C)OC(=O)N1CC2CC1CC2=O ; CC(C)(C)OC(=O)N1C[C@@H]2C[C@H]1C[C@@H]2O ; CC(C)(C)OC(=O)N1C[C@@H]2C[C@H]1C[C@H]2OC(=O)c1ccccc1]

To a stirred solution of racemic tert-butyl 5-oxo-2-azabicyclo[2.2.1]heptane-2-carboxylate (100 g, 473.4 mmol, 1.0 equiv) in MeOH (1000 mL) NaBH₄ (17.38 g, 473.4 mmol, 1.0 equiv) is added portion wise at 0° C. Then, the reaction mixture is allowed to RT and stirred for 3 hours. The reaction mixture is quenched with ice-cold water and extracted with DCM. The combined organic layers are dried over sodium sulphate, filtered and concentrated under reduced pressure to get a crude racemic mixture of tert-butyl (1S,4S,5S)-5-hydroxy-2-azabicyclo[2.2.1]heptane-2-carboxylate and tert-butyl (1R,4R,5R)-5-hydroxy-2-azabicyclo[2.2.1]heptane-2-carboxylate, which is used without further purification.

To a stirred solution of the intermediate racemic mixture of tert-butyl (1S,4S,5S)-5-hydroxy-2-azabicyclo[2.2.1]heptane-2-carboxylate and tert-butyl (1R,4R,5R)-5-hydroxy-2-azabicyclo[2.2.1]heptane-2-carboxylate (190.00 g, 891 mmol, 1.0 equiv) in THF (3800 mL) benzoic acid (163.2 g, 1336.3 mmol, 1.5 equiv) and PPh₃ (350.37 g, 1336.3 mmol, 1.5 equiv) are added at 0° C. The resulting reaction mixture is stirred for 10 min at 0° C. and DIAD (265 mL, 1336.3 mmol, 1.5 equiv.) is added dropwise at same temperature. The reaction mixture is stirred for 16 h at RT. The reaction mixture is quenched with NaHCO₃ solution and extracted with ethyl acetate. The combined organic layers are dried over sodium sulphate, filtered and concentrated under reduced pressure The crude compound is purified by NP chromatography to afford racemic A-4a. (HPLC method Y, tᵣₑₜ=2.40 min; [M+H-^{t}butyl]=262).

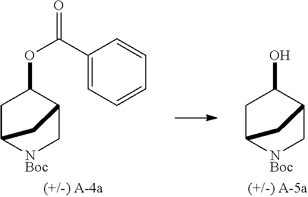 [SMILES: CC(C)(C)OC(=O)N1C[C@@H]2C[C@H]1C[C@H]2OC(=O)c1ccccc1 ; CC(C)(C)OC(=O)N1C[C@@H]2C[C@H]1C[C@H]2O]

To a stirred solution of racemic A-4a (210 g, 661.7 mmol, 1.0 equiv.) in MeOH (2100 mL) 1M KOH in MeOH (992.5 mL, 992.5 mmol, 1.5 equiv.) is added at 0° C. and stirred for 4 h at RT. The reaction mixture is quenched with ice-cold water and extracted with ethyl acetate.

The combined organic layers are dried over sodium sulphate, filtered and concentrated under reduced pressure to get crude compound, which is purified by NP chromatography to afford racemic A-5a. (HPLC method Y, t_{ref}=1.5 min; [M+H-^{t}butyl]=158)

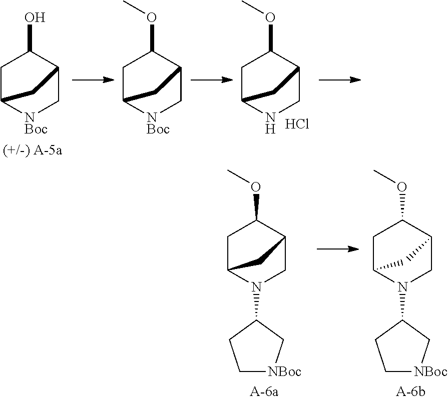

To a stirred solution of racemic A-5a (150 g, 703.3 mmol, 1 equiv.) in THF (1500 mL) 60% NaH (50.64 g, 2110 mmol, 3 equiv.) is added portion wise at 0° C. and stirred for 30 min at RT. Then, Mel (57 mL, 914 mmol, 1.3 equiv.) in THF (15 mL) is added dropwise at 0° C. and stirred for 4 h at RT. The reaction mixture is quenched with ice-cold water and extracted with ethyl acetate. The combined organic layers are dried over sodium sulphate, filtered and concentrated under reduced pressure to get a crude racemic mixture of tert-butyl (1S,4S,5R)-5-methoxy-2-azabicyclo[2.2.1]heptane-2-carboxylate and tert-butyl (1R,4R,5S)-5-methoxy-2-azabicyclo[2.2.1]heptane-2-carboxylate.

The intermediate racemic mixture of tert-butyl (1S,4S,5R)-5-methoxy-2-azabicyclo[2.2.1]heptane-2-carboxylate and tert-butyl (1R,4R,5S)-5-methoxy-2-azabicyclo[2.2.1]heptane-2-carboxylate (180.00 g, 71.92 mmol, 1 equiv.) is redissolved in DCM (1800 mL). 4M HCl in dioxane (360 mL, 1440 mmol, 1.82 equiv.) is added at 0° C. and stirred for 16 h at RT. The solvent is removed under reduced pressure to get a crude racemic mixture of (1S,4S,5R)-5-methoxy-2-azabicyclo[2.2.1]heptane hydrochloride and (1R,4R,5S)-5-methoxy-2-azabicyclo[2.2.1]heptane hydrochloride.

To a stirred solution of (R)-tert-butyl 3-(tosyloxy)pyrrolidine-1-carboxylate (150.00 g, 439 mmol, 1 equiv) and a racemic mixture of (1S,4S,5R)-5-methoxy-2-azabicyclo[2.2.1]heptane hydrochloride and (1R,4R,5S)-5-methoxy-2-azabicyclo[2.2.1]heptane hydrochloride (107.84 g, 659 mmol, 1.5 equiv) in acetonitrile (1500 mL) is added K₂CO₃ (242.87 g, 1757.3 mmol, 4 equiv) at RT and stirred for 16 h at 90° C. The solvent is removed under reduced pressure and the obtained residue is diluted ethyl acetate washed with water. The organic layer is dried over Na₂SO₄ and concentrated under reduced pressure to get crude mixture, which is purified by NP chromatography. Then, the mixture is separated with Method SFC 2 to afford A-6a and A-6b. A-6c and A-6d are prepared analogously by replacing methyl iodide with the corresponding 1-iodo-2-methoxyethane.

The following intermediate compounds (Table 4) are obtained analogously.

TABLE 4

| | | | | HPLC |
|---|---|---|---|---|
| # | Structure | tᵣₑₜ [min] | [M + H]⁺ | method |
| A-6a | 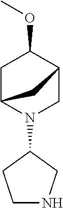 | 1.24 | 297 | Y |
| A-6b | 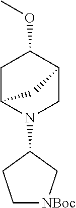 [SMILES: CO[C@H]1C[C@H]2C[C@@H]1CN2[C@H]1CCN(C(=O)OC(C)(C)C)C1] | 1.23 | 297 | Y |
| A-6c | 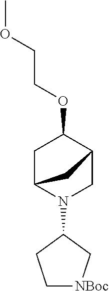 [SMILES: COCCO[C@@H]1C[C@@H]2C[C@H]1CN2[C@H]1CCN(C(=O)OC(C)(C)C)C1] | 4.599 | 341 | SFC-2 |
| A-6d | 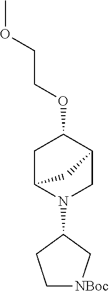 [SMILES: COCCO[C@H]1C[C@H]2C[C@@H]1CN2[C@H]1CCN(C(=O)OC(C)(C)C)C1] | 4.066 | 341 | SFC-2 |

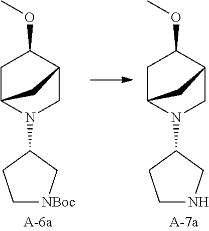 [SMILES: CO[C@@H]1C[C@@H]2C[C@H]1CN2[C@H]1CCN(C(=O)OC(C)(C)C)C1 ; CO[C@@H]1C[C@@H]2C[C@H]1CN2[C@H]1CCNC1]

To a stirred solution of A-6a (13.00 g, 43.8 mmol) in dichloromethane (130 mL) is added 4M HCl in dioxane (66 mL, 104 mmol, 2.37 mmol) at 0° C. and stirred for 16 h at RT. The solvent is removed under reduced pressure and then triturated with n-pentane to get of A-7a (H PLC method Y, tᵣₑₜ=2.40 min; [M+H-^{t}butyl]=262) as brown gummy liquid.

The following intermediate compounds (Table 5) are obtained analogously.

TABLE 5

| | | | | HPLC |
|---|---|---|---|---|
| # | Structure | tᵣₑₜ [min] | [M + H]⁺ | method |
| A-7a | 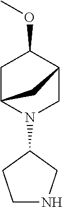 | 1.20 | 197 | AD |
| A-7b | 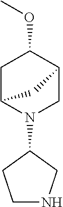 | 1.19 | 197 | AD |
| A-7c | 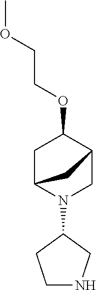 [SMILES: COCCO[C@@H]1C[C@@H]2C[C@H]1CN2[C@H]1CCNC1] | 2.21 | 241 | AG |
| A-7d | 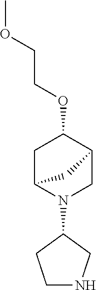 [SMILES: COCCO[C@H]1C[C@H]2C[C@@H]1CN2[C@H]1CCNC1] | 2.18 | 241 | AG |

Synthesis of N,N-bis-Substituted 2-amino Azetidines

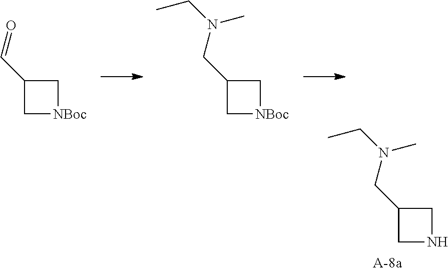

A solution of tert-butyl 3-formylazetidine-1-carboxylate (500 mg, 2.7 mmol, 1.0 equiv.) in dry methanol (7.5 mL) is prepared. To this, acetic acid (0.081 g, 1.36 mmol, 0.50 equiv.) and N-ethylmethylamine (329 mg, 5.4 mmol, 2.0 equiv.) are added. This mixture is stirred at room temperature for 10 minutes. Sodium triacetoxyborohydride (1.18 g, 5.4 mmol, 2.0 equiv.) is then added to the reaction mixture, which is stirred at room temperature for 1 h. The reaction mixture was filtered and evaporated to dryness. The residue was redissolved in dioxane (4 mL) and 6 M HCl (2.5 mL, 14.5 mmol, 10 equiv) was added. The reaction mixture is directly loaded on Isolute and purified by chromatography to give the product A-8a (HPLC method A, tᵣₑₜ=0.71 min; [M+H]=202).

The following intermediate compounds (Table 6) are obtained analogously.

TABLE 6

| | | | | HPLC |
|---|---|---|---|---|
| # | Structure | tᵣₑₜ [min] | [M + H]⁺ | method |
| A-8a | 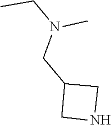 | 0.71 | 202 | A |
| A-8b | 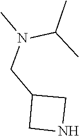 | 0.73 | 142 | A |
| A-8c | 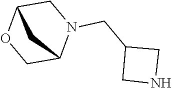 [SMILES: C1NCC1CN1C[C@@H]2C[C@H]1CO2] | 0.74 | 168 | A |
| A-8d | 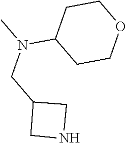 | 0.69 | 184 | A |
| A-8e | 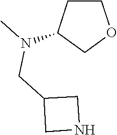 | 0.72 | 170 | A |

### Synthesis of Substituted Heterocyclic Anilines

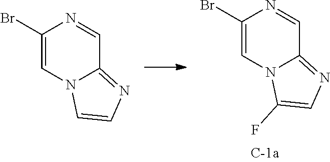 [SMILES: Brc1cn2ccnc2cn1 ; Fc1cnc2cnc(Br)cn12]

A solution of 6-bromoimidazo[1,2-a]pyrazine (24 g, 121 mmol, 1.0 equiv.) in acetonitrile (ACN, 240 mL) is cooled to 0° C. Sodium hydride (NaH, 7.27 g, 182 mmol, 1.5 equiv.) is added to the solution. The reaction mixture is stirred at room temperature for 30 minutes. Following this, Selectfluor (85.87 g, 242 mmol, 2.0 equiv.) is added in acetonitrile (480 mL). The reaction mixture is stirred at room temperature for 16 hours.

The reaction mixture is quenched with water and extracted with ethyl acetate. The organic layer is dried over sodium sulfate (Na₂SO₄) and the solvent is evaporated under reduced pressure. The crude product is purified using neutral alumina and eluted in 4% ethyl acetate/hexane to afford the product, 6-bromo-3-fluoro-imidazo[1,2-a]pyrazine C-1a (HPLC method AF, tᵣₑₜ=1.17 min; [M+H]=216).

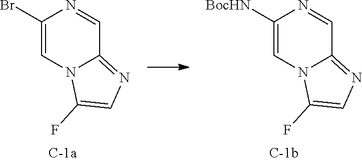 [SMILES: Fc1cnc2cnc(Br)cn12 ; CC(C)(C)OC(=O)Nc1cn2c(F)cnc2cn1]

A solution of 6-bromo-3-fluoro-imidazo[1,2-a]pyrazine (4.75 g, 22 mmol, 1.0 equiv.) in 1,4-dioxane (90.5 mL) is stirred. To this, cesium carbonate (Cs₂CO₃, 0.79 g, 33 mmol, 1.5 equiv.), tert-butyl carbonate (5.87 g, 44 mmol, 2.0 equiv.), and Xantphos (12.72 g, 22 mmol, 1.0 equiv.) are added. The mixture is degassed for 5 minutes. Following this, palladium(II) acetate (Pd(OAc)₂, 2.3 mL, 22 mmol, 1.0 equiv.) is added and the mixture is degassed for another 5 minutes. The reaction mixture is then stirred at 100° C. for 3 hours.

The reaction mixture is filtered through a Celite bed and the solvent is evaporated under reduced pressure. The crude product is purified using basic silica and eluted in 27% ethyl acetate/hexane to afford the product, tert-butyl N-(3-fluoroimidazo[1,2-a]pyrazin-6-yl)carbamate C-1b (HPLC method X, tᵣₑₜ=2.62 min; [M+H]=253).

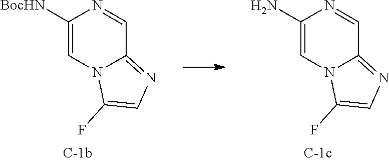 [SMILES: CC(C)(C)OC(=O)Nc1cn2c(F)cnc2cn1 ; Nc1cn2c(F)cnc2cn1]

A solution of tert-butyl N-(3-fluoroimidazo[1,2-a]pyrazin-6-yl)carbamate (4.00 g, 15.9 mmol, 1.0 equiv.) in dichloromethane (DCM, 40 mL) is stirred. To this, trifluoroacetic acid (12 mL) is added. The reaction mixture is stirred at room temperature for 4 hours.

The reaction mixture is then evaporated to afford the crude product. This is diluted with tetrahydrofuran (THF, 10 mL) and a 20% sodium hydroxide (NaOH) solution (30 mL) is added. The mixture is stirred for 1 hour and then extracted with DCM (3×15 mL). The organic layer is washed with water (2×10 mL), then a brine solution (10 mL), and dried over sodium sulfate (Na₂SO₄). The solvent is evaporated under reduced pressure to afford a pale brown solid. This is washed with diethyl ether (3×6 mL) and dried to afford the product, 3-fluoroimidazo[1,2-a]pyrazin-6-amine C-1c (HPLC method X, tᵣₑₜ=2.14 min; [M+H]=167).

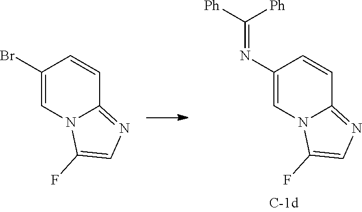 [SMILES: Fc1cnc2ccc(Br)cn12 ; Fc1cnc2ccc(N=C(c3ccccc3)c3ccccc3)cn12]

A solution of 6-bromo-3-fluoro-imidazo[1,2-a]pyridine (21.00 g, 97.7 mmol, 1.0 equiv.) in dioxane (420 mL) is stirred at room temperature and degassed with nitrogen. To this, sodium tert-butoxide (NaOtBu, 14.92 g, 155 mmol, 1.59 equiv.), benzophenone imine (27.08 g, 149 mmol, 1.53 equiv.), BINAP (15.94 g, 19.5 mmol, 0.20 equiv.), and Pd₂(dba)₃ (8.94 g, 9.77 mmol, 0.10 equiv.) are added. The reaction mixture is heated to 80° C. for 2 hours.

After the starting material is consumed (as monitored by TLC and LCMS), the reaction mixture is quenched with water and extracted with ethyl acetate (2×30 mL). The organic layer is separated, dried over anhydrous sodium sulfate (Na₂SO₄), and the solvent is evaporated under reduced pressure to afford the crude product. This is purified by column chromatography on basic silica gel, eluting with 45% ethyl acetate in petroleum ether to afford the product, N-(3-fluoroimidazo[1,2-a]pyridin-6-yl)-1,1-diphenyl-methanimine C-1d (HPLC method X, tᵣₑₜ=1.00 min; [M+H]=316).

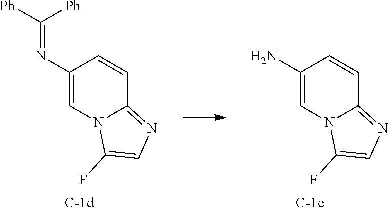 [SMILES: Fc1cnc2ccc(N=C(c3ccccc3)c3ccccc3)cn12 ; Nc1ccc2ncc(F)n2c1]

A solution of N-(3-fluoroimidazo[1,2-a]pyridin-6-yl)-1,1-diphenyl-methanimine (0.10 g, 0.317 mmol, 1.0 equiv.) in tetrahydrofuran (THF, 2 mL) is stirred at 0° C. To this, 1N hydrochloric acid (HCl, 4.0 mL, 0.317 mmol, 1.0 equiv.) is added. The reaction mixture is then stirred at room temperature for 1 hour.

The reaction mixture is dissolved in dichloromethane (50 mL) and the organic layer is separated. The aqueous layer is basified with potassium carbonate (K₂CO₃) and extracted with a 20% methanol (MeOH) in DCM solution (2×100 mL). The organic layers are combined, concentrated under reduced pressure, and dried. The solid is washed with diethyl ether and dried to afford the product, 3-fluoroimidazo[1,2-a]pyridin-6-amine C-1e (HPLC method AD, tᵣₑₜ=2.21 min; [M+H]=152).

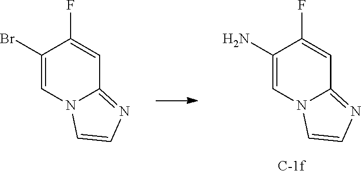 [SMILES: Fc1cc2nccn2cc1Br ; Nc1cn2ccnc2cc1F]

In a sealed tube, 6-bromo-7-fluoro-imidazo[1,2-a]pyridine (2.00 g, 9.30 mmol, 1.0 equiv.) is dissolved in dimethyl sulfoxide (DMSO, 20 mL) at room temperature. To this, L-proline (0.43 g, 3.72 mmol, 0.4 equiv.), potassium carbonate (K₂CO₃, 3.93 g, 14.0 mmol, 1.5 equiv.), copper(I) iodide (CuI, 0.35 g, 1.86 mmol, 0.2 equiv.), and 25% aqueous ammonia (10 mL) are added. The reaction mixture is stirred at 90° C. for 16 hours.

The reaction progress is monitored by LCMS. After completion, the reaction mixture is diluted with ice-cold water (10 mL) and extracted with ethyl acetate (EtOAc). The organic layer is washed with brine, filtered, and the solvent is evaporated under reduced pressure to afford the crude product. This is purified by column chromatography on basic silica gel, eluting with a 5% methanol (MeOH) in dichloromethane (DCM) solution to afford the product, 7-fluoroimidazo[1,2-a]pyridin-6-amine C-1f (HPLC method AF, tᵣₑₜ=5.92 min; [M+H]=151).

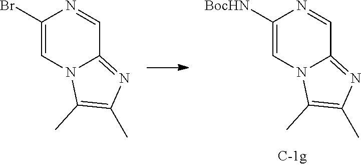 [SMILES: Cc1nc2cnc(Br)cn2c1C ; Cc1nc2cnc(NC(=O)OC(C)(C)C)cn2c1C]

A solution of 6-bromo-2,3-dimethyl-imidazo[1,2-a]pyrazine (18.00 g, 79.6 mmol, 1.0 equiv.) in 1,4-dioxane (180 mL) is stirred. To this, tert-butyl carbamate (18.65 g, 159 mmol, 2.0 equiv.) and cesium carbonate (77.82 g, 239 mmol, 3.0 equiv.) are added. The reaction mixture is degassed with argon for 5 minutes. Then, palladium(II) acetate (1.79 g, 7.96 mmol, 0.1 equiv.) and xanthphos (9.20 g, 15.9 mmol, 0.2 equiv.) are added. The reaction mixture is stirred at 100° C. for 16 hours and the progress is monitored by LCMS.

After completion, the reaction mixture is filtered through a Celite bed and washed with ethyl acetate. The filtrate is concentrated under reduced pressure to afford the crude product. This is purified by column chromatography on neutral alumina, eluting with a 60-70% ethyl acetate in hexane solution to afford the product, tert-butyl N-(2,3-dimethylimidazo[1,2-a]pyrazin-6-yl)carbamate, as a brown solid (7.00 g, 24.0 mmol, 30.17% yield) C-1g (HPLC method AD, tᵣₑₜ=2.56 min: [M+H]=249).

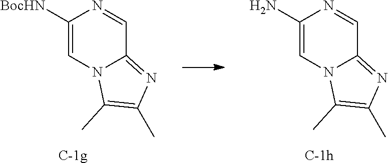

A solution of tert-butyl N-(2,3-dimethylimidazo[1,2-a]pyrazin-6-yl)carbamate C-1g (100 mg, 0.381 mmol, 1.0 equiv.) in 4M HCl in dioxane (3 mL) is prepared at 0° C. The reaction mixture is stirred at room temperature for 12 hours and the progress is monitored by LCMS.

After completion, the reaction mixture is concentrated to dryness. The residue is dissolved in water and the pH is adjusted to 8-9 with a saturated sodium carbonate solution. The product is extracted with dichloromethane (DCM). The organic layer is washed with water and then with a saturated brine solution. It is then dried over sodium sulfate (Na₂SO₄) and the solvent is evaporated to afford the product, 2,3-dimethylimidazo[1,2-a]pyrazin-6-amine, as a solid C-1h (HPLC method AD, tᵣₑₜ=2.15 min; [M+H]=163).

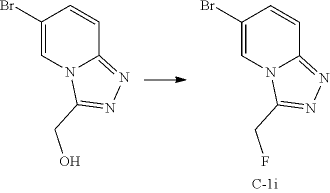 [SMILES: OCc1nnc2ccc(Br)cn12 ; FCc1nnc2ccc(Br)cn12]

A solution of (6-bromo-[1,2,4]triazolo[4,3-a]pyridin-3-yl)methanol (50 mg, 0.219 mmol, 1.0 equiv.) in dichloromethane (DCM, 2 mL) is stirred at room temperature. To this, diethylaminosulfur trifluoride (DAST, 71 mg, 0.439 mmol, 2.0 equiv.) is added at 0° C. The reaction mixture is stirred at room temperature for 16 hours. The progress of the reaction is monitored by thin-layer chromatography (TLC), which shows the desired product.

After completion, the reaction mixture is diluted with water (10 mL) and extracted with DCM (2×20 mL). The organic layer is washed with a brine solution, filtered, and the solvent is evaporated under reduced pressure to afford the crude product, 6-bromo-3-(fluoromethyl)-[1,2,4]triazolo[4,3-a]pyridine, as a brown solid C-1i (HPLC method AD, tᵣₑₜ=2.36 min; [M+H]=167).

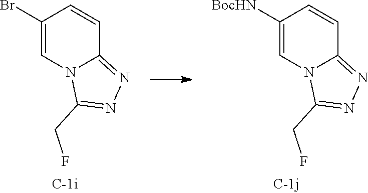 [SMILES: FCc1nnc2ccc(Br)cn12 ; CC(C)(C)OC(=O)Nc1ccc2nnc(CF)n2c1]

A solution of 6-bromo-3-(fluoromethyl)-[1,2,4]triazolo[4,3-a]pyridine (50 mg, 0.217 mmol, 1.0 equiv.) in 1,4-dioxane (2 mL) is stirred at room temperature. To this, tert-butyl carbamate (38 mg, 0.326 mmol, 1.5 equiv.) and cesium carbonate (Cs₂CO₃, 177 mg, 0.543 mmol, 2.5 equiv.) are added. The reaction mixture is degassed under a nitrogen atmosphere for 10 minutes. Then, Xantphos (13 mg, 0.0217 mmol, 0.1 equiv.) and palladium(II) acetate (4.9 mg, 0.0217 mmol, 0.1 equiv.) are added. The resulting reaction mixture is stirred at 80° C. for 16 hours. The progress of the reaction is monitored by thin-layer chromatography (TLC), which shows complete conversion of the starting material.

After completion, the reaction mixture is diluted with water (30 mL) and extracted with ethyl acetate (EtOAc, 3×50 mL). The organic layer is washed with a brine solution, filtered, and the solvent is evaporated under reduced pressure to afford the crude product, tert-butyl N-[3-(fluoromethyl)-[1,2,4]triazolo[4,3-a]pyridin-6-yl]carbamate, as an off-white solid C-1j (HPLC method AD, tᵣₑₜ=2.47 min; [M+H]=267).

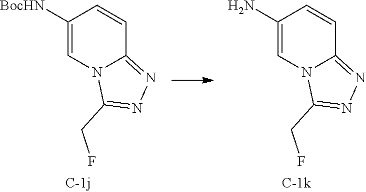 [SMILES: CC(C)(C)OC(=O)Nc1ccc2nnc(CF)n2c1 ; Nc1ccc2nnc(CF)n2c1]

A solution of tert-butyl N-[3-(fluoromethyl)-[1,2,4]triazolo[4,3-a]pyridin-6-yl]carbamate (100 mg, 0.214 mmol, 1.0 equiv.) in dichloromethane (DCM, 5 mL) is stirred at 0° C. To this, 4N hydrochloric acid (HCl) in dioxane (0.054 mL, 0.214 mmol, 1.0 equiv.) is added dropwise. The resulting reaction mixture is stirred at room temperature for 16 hours. The progress of the reaction is monitored by thin-layer chromatography (TLC), which shows complete conversion of the starting material.

After completion, the reaction mixture is concentrated under reduced pressure. The crude product is washed with diethyl ether and the solvent is evaporated under reduced pressure to afford the crude product, 3-(fluoromethyl)-[1,2,4]triazolo[4,3-a]pyridin-6-amine, as an off-white solid C-1k (H PLC method AD, tᵣₑₜ=0.87 min; [M+H]=167).

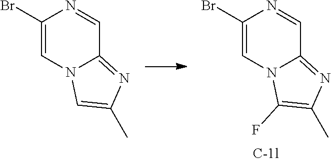

A solution of 6-bromo-2-methyl-imidazo[1,2-a]pyrazine (30.00 g, 141 mmol, 1.0 equiv.) in 1,2-dichloroethane (DCE, 750 mL) is stirred at 0° C. under a nitrogen atmosphere. To this, Selectfluor (150.36 g, 424 mmol, 3.0 equiv.) is added. The resulting reaction mixture is stirred at 70° C. for 36 hours. The progress of the reaction is monitored by thin-layer chromatography (TLC), which shows the presence of the starting material and a new spot at non-polar conditions. After completion, the reaction mixture is filtered and the filtrate is concentrated under vacuum to afford the crude product as a brown solid. The crude product is purified using chromatex silica, eluting with 12-15% ethyl acetate in hexane. This affords the product, 6-bromo-3-fluoro-2-methyl-imidazo[1,2-a]pyrazine, as a yellow solid (4 g, 14.4 mmol, 11.307% yield) C-1l (HPLC method AF, tᵣₑₜ=3.92 min; [M+H]=229).

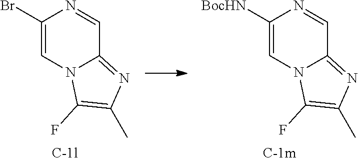 [SMILES: Cc1nc2cnc(Br)cn2c1F ; Cc1nc2cnc(NC(=O)OC(C)(C)C)cn2c1F]

A solution of 6-bromo-3-fluoro-2-methyl-imidazo[1,2-a]pyrazine (22.50 g, 97.8 mmol, 1.0 equiv.) in 1,4-dioxane (225 mL) is stirred under a nitrogen atmosphere. To this, tert-butyl carbamate (34.38 g, 293 mmol, 3.0 equiv.), cesium carbonate (127.47 g, 391 mmol, 4.0 equiv.), and BrettPhos Pd G3 (5.32 g, 5.87 mmol, 0.0600 equiv.) are added. The reaction mixture is stirred at 100° C. for 16 hours. After completion, the reaction mixture is filtered and the filtrate is concentrated under vacuum. The crude product is washed with water and extracted with dichloromethane (DCM, 2×5 mL). The combined organic layers are dried over anhydrous sodium sulfate, filtered, and concentrated under vacuum. The crude product is purified by flash column chromatography (FCC) on silica gel, eluting with 42% ethyl acetate in petroleum ether. This affords the product, tert-butyl N-(3-fluoro-2-methyl-imidazo[1,2-a]pyrazin-6-yl)carbamate, as an off-white solid C-1m (HPLC method AF, tᵣₑₜ=4.42 min; [M+H]=267).

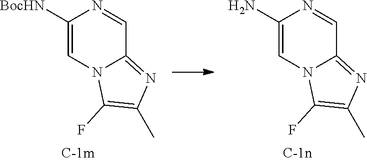 [SMILES: Cc1nc2cnc(NC(=O)OC(C)(C)C)cn2c1F ; Cc1nc2cnc(N)cn2c1F]

A solution of tert-butyl N-(3-fluoro-2-methyl-imidazo[1,2-a]pyrazin-6-yl)carbamate (20.00 g, 75.1 mmol, 1.0 equiv.) in dichloromethane (DCM, 600 mL) is stirred at 0° C. under a nitrogen atmosphere. To this, trifluoroacetic acid (TFA, 80 mL) is added. The resulting reaction mixture is stirred for 16 hours. After completion, the reaction mixture is concentrated under vacuum. The crude product is dissolved in water and washed with DCM (2×100 mL). The aqueous layer is basified with solid sodium bicarbonate (until pH ˜8.5-9) at cold conditions, and the product is extracted with 5% methanol in DCM (3×150 mL). The combined organic layers are dried over anhydrous sodium sulfate, filtered, and concentrated under vacuum.

The crude product is washed with diethyl ether (2×30 mL) and dried to afford the product, 3-fluoro-2-methyl-imidazo[1,2-a]pyrazin-6-amine, as an off-white solid C-1n (HPLC method AF, tᵣₑₜ=3.37 min; [M+H]=167).

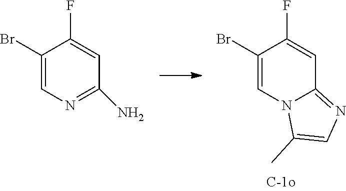

A solution of 2-bromo-1,1-diethoxy-propane (76.26 g, 361 mmol, 2.3 equiv.) is treated with 1HCl (38.1 mL) and stirred for 1 hour at 90° C. The reaction mixture is cooled to room temperature and treated with solid sodium bicarbonate until pH 7. To this, 5-bromo-4-fluoro-pyridin-2-amine (30.00 g, 157 mmol, 1.0 equiv.) in methanol (70 mL) is added. The reaction mixture is stirred for 8 hours at 90° C. After completion, the reaction mixture is concentrated under vacuum. The crude product is basified with sodium bicarbonate, and the reaction mixture is extracted with ethyl acetate (2×150 mL). The combined organic layers are dried over anhydrous sodium sulfate, filtered, and concentrated under vacuum.

The product, 6-bromo-7-fluoro-3-methyl-imidazo[1,2-a]pyridine, is obtained as a crude solid C-1o (HPLC method AD, tᵣₑₜ=2.50 min; [M+H]=228).

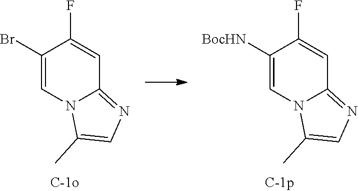 [SMILES: Cc1cnc2cc(F)c(Br)cn12 ; Cc1cnc2cc(F)c(NC(=O)OC(C)(C)C)cn12]

A solution of 6-bromo-7-fluoro-3-methyl-imidazo[1,2-a]pyridine C-1o (20.00 g, 87.3 mmol, 1.0 equiv.) in toluene (300 mL) is stirred under nitrogen for 10 minutes. To this, tert-butyl carbamate (61.38 g, 524 mmol, 6.0 equiv.), cesium carbonate (227.60 g, 699 mmol, 8.0 equiv.), and BrettPhos Pd G3 (7.92 g, 8.73 mmol, 0.1 equiv.) are added. The reaction mixture is stirred for 16 hours at 120° C. The progress of the reaction is monitored by thin-layer chromatography (TLC), using 50% ethyl acetate in petroleum ether.

After completion, the reaction mixture is filtered and concentrated under vacuum. The crude product is washed with water and extracted with dichloromethane (DCM, 2×50 mL). The combined organic layers are dried over anhydrous sodium sulfate, filtered, and concentrated under vacuum.

The crude product is purified by column chromatography on basic silica, using 30-40% ethyl acetate in petroleum ether as the eluent. The pure fractions are combined and concentrated under vacuum to afford the product, tert-butyl N-(7-fluoro-3-methyl-imidazo[1,2-a]pyridin-6-yl)carbamate, as an off-white solid C-1p (H PLC method AD, tᵣₑₜ=2.54 min; [M+H]=266).

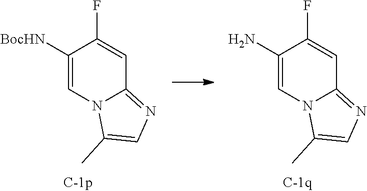 [SMILES: Cc1cnc2cc(F)c(NC(=O)OC(C)(C)C)cn12 ; Cc1cnc2cc(F)c(N)cn12]

A solution of tert-butyl N-(7-fluoro-3-methyl-imidazo[1,2-a]pyridin-6-yl)carbamate (10.20 g, 38.4 mmol, 1.0 equiv.) in dichloromethane (DCM, 102 mL) is cooled to 0° C. under a nitrogen atmosphere. To this, 4 M dioxane HCl (51 mL) is added. The reaction mixture is stirred for 16 hours. After completion, the reaction mixture is concentrated under vacuum. The crude product is dissolved in water and washed with DCM (2×100 mL). The aqueous layer is basified with solid sodium bicarbonate (pH ˜8.5-9) at cold conditions, and the product is extracted with 5% methanol in DCM (3×150 mL). The combined organic layers are dried over anhydrous sodium sulfate, filtered, and concentrated under vacuum. The crude product is purified by flash column chromatography on chromatex, using 2-4% methanol in DCM as the eluent. The product, 7-fluoro-3-methyl-imidazo[1,2-a]pyridin-6-amine C-1q, is obtained as an off-white solid (4.15 g, 22.6 mmol, 58.68% yield) (HPLC method AD, tᵣₑₜ=2.32 min; [M+H]=166).

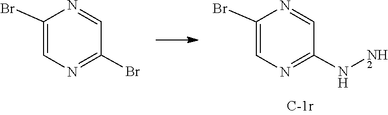 [SMILES: Brc1cnc(Br)cn1 ; NNc1cnc(Br)cn1]

2,5-Dibromopyrazine (10 g, 42 mmol, 1 equiv.) is dissolved in isopropanol (100 mL) and hydrazine hydrate (14 mL, 210 mmol, 5 equiv.) is added. The reaction mixture is heated to 65° C. and stirred for 16 h. Upon completion of the reaction, the reaction is cooled to 0° C. and the precipitate is filtered, washed with isopropanol and dried to give C-1r (5 g, 21.7 mmol; TLC: 50% EtOAc in Hexane, R_{f}: 0.5; HPLC method AC; tᵣₑₜ=2.3 min; [M+H]+=189).

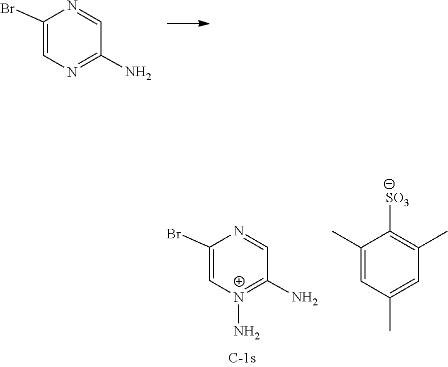

5-Bromopyrazin-2-amine (34 g, 195 mmol, 1 equiv.) is dissolved in DCM (6.75 mL) at 0° C. then O-(mesitylsulfonyl)hydroxylamine (50 g, 234 mmol, 1.2 equiv.) is added. The reaction mixture is stirred at 30° C. for 16 h. Upon complete conversion, the reaction mixture is filtered and the precipates is dried under vacuum to obtain C-1s (60 g, 72 mmol; TLC: 70% EtOAc in Hexane, R_{f}: 0.3; HPLC method: AC; tᵣₑₜ=0.86 min; [M+H]+=189).

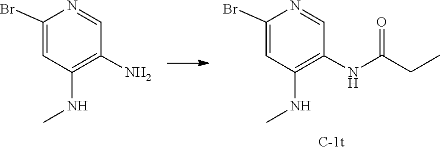

To a stirred solution of 6-Bromo-N4-methylpyridine-3,4-diamine (43 g, 213 mmol, 1 equiv.) is dissolved in propionic acid (16 mL, 213 mmol, 1 equiv.). EDC-HCl (31 g, 319 mmol, 1.5 equiv.) and DIPEA (56 mL, 319 mmol, 1.5 equiv.) are added and the reaction mixture is stirred at room temperature for 16 h. Upon complete conversion, the reaction is diluted with DCM (200 mL) and washed three times with water then with brine. The combined organic layers are dried over Na₂SO₄, filtered, and concentrated in vacuo. The crude material is purified by NP-chromatography to give C-1t (30 g, 101 mmol; TLC: 10% MeOH in DCM, R_{f}: 0.4; HPLC method: AC; tᵣₑₜ=2.3 min; [M+H]+=258)

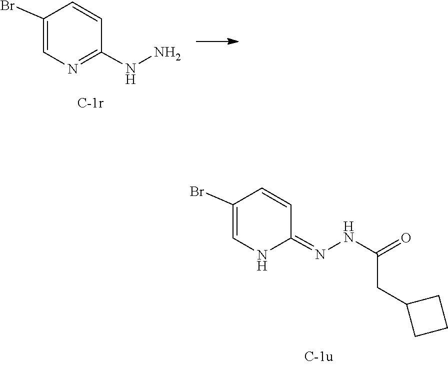 [SMILES: NNc1ccc(Br)cn1 ; O=C(CC1CCC1)N/N=c1\ccc(Br)c[nH]1]

5-bromo-2-hydrazineylpyridine C-1r (10 g, 53 mmol, 1 equiv.) is dissolved in toluene (100 mL) and 2-cylcobutylacetic acid (6.1 g, 53 mmol, 1 equiv.) is added at 0° C. Next, reaction mixture is heated to 120° C. and stirred for 3 days. Upon complete conversion the reaction mixture is concentrated in vacuo and the crude material used without further purification (4.5 g, 4.6 mmol; TLC: 5% MeOH in DCM, R_{f}: 0.3; HPLC method: AC; tᵣₑₜ=1.25 min; [M+H]+=284).

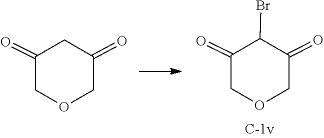 [SMILES: O=C1COCC(=O)C1 ; O=C1COCC(=O)C1Br]

A solution of tetrahydropyran-3,5-dione (10 g, 88 mmol, 1 equiv.) in DMF (420 mL) is stirred to 15 minutes. Next, NBS (17.2 g, 96 mmol, 1.1 equiv.) is added and the reaction mixture is stirred at room temperature for 4 h. Upon complete conversion, the reaction mixture is diluted with water and extracted with EtOAc. The organic layers are washed with brine, dried over Na₂SO₄, filtered, and concentrated in vacuo. The crude material is purified by NP chromatography to give C-1v (6 g, 30 mmol; TLC: 50% EtOAc in Hexane, R_{f}: 0.5; HPLC method: AC; tᵣₑₜ=0.8 min; [M+H]+=193).

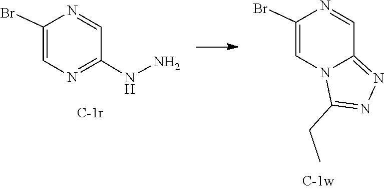

C-1r (2 g, 10.6 mmol, 1 equiv.) is dissolved in DCM (20 mL) and propionaldehyde (0.91 mL, 12.7 mmol, 1.2 equiv.) is added at room temperature and stirred for 30 minutes. Iodobenzene diacetate (3.4 g, 10.6 mmol, 1 equiv.) is added and the reaction mixture is stirred at room temperature for 6 h. Upon completion of the reaction, the reaction mixture is diluted with DCM (50 mL) and basified with sat. aqueous sodium hydrogen carbonate to pH 7. The organic layers are washed with water (25 mL) and dried over Na₂SO₄, filtered and concentrated in vacuo. The crude material is purified by NP chromatography to product C-1w (500 mg, 2.1 mmol; TLC: 50% EtOAc in Hexane, R_{f}: 0.5; HPLC method: AC; tᵣₑₜ=2.3 min; [M+H]+=189).

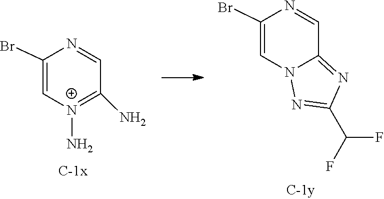 [SMILES: Nc1cnc(Br)c[n+]1N ; FC(F)c1nc2cnc(Br)cn2n1]

A solution of C-1x (60 g, 154 mmol, 1 equiv.) in difluoroacetic acid (120 mL) is heated to 130° C. and stirred for 4 h. Upon completion of the reaction, the reaction mixture is cooled and concentrated in vacuo. The crude material is dissolved in DCM and washed sequentially with water and brine. The combined organic layers are dried over Na₂SO₄, filtered, and concentrated in vacuo. The crude product is purified by NP-chromatography to give C-1y (15 g, 48 mmol; TLC: 20% EtOAc in Hexane, R_{f}: 0.6; HPLC method AC; tᵣₑₜ=2.5 min; [M+H]+=250).

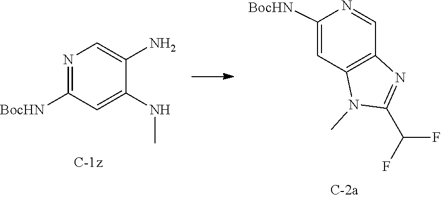 [SMILES: CNc1cc(NC(=O)OC(C)(C)C)ncc1N ; Cn1c(C(F)F)nc2cnc(NC(=O)OC(C)(C)C)cc21]

A solution of tert-butyl (5-amino-4-(methylamino)pyridin-2-yl)carbamate C-1z (32 g, 134 mmol, 1 equiv.) in THF (320 mL) is stirred at 0° C. and Et₃N (39 mL, 268 mmol, 2 equiv.) is added. After 5 minutes, difluoroacetic anhydride (28 g, 161 mmol, 1.2 equiv.) is added at the same temperature. The reaction is heated to 80° C. and stirred for 16 h. Upon complete conversion, the reaction is concentrated in vacuo. The crude material is diluted with water and extracted twice with EtOAc. The combined organic layers are washed with brine, dried over Na₂SO₄, filtered, and concentrated in vacuo. The crude material is triturated with Et₂O (2×25 mL) and the solid filtered and dried under vacuum to give C-2a (12.5 g, 41.9 mmol; TLC: 50% EtOAc in Hexane, R_{f}: 0.5; AD; tᵣₑₜ=1.9 min; [M+H]+=299).

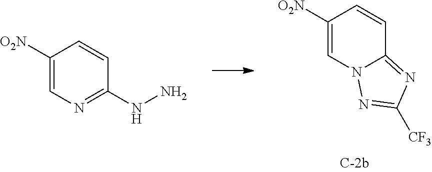 [SMILES: NNc1ccc([N+](=O)[O-])cn1 ; O=[N+]([O-])c1ccc2nc(C(F)(F)F)nn2c1]

5-Nitro-2-pyridylhydrazine (10 g, 65 mmol, 1 equiv.) is dissolved in TFA (50 mL, 614 mmol, 9.5 equiv.) and the reaction mixture is heated to 120° C. and stirred for 16 h. Upon complete conversion, the reaction mixture is concentrated in vacuo. The residue is basified with sat. aq. NaHCO₃, extracted twice with EtOAc and dried over Na₂SO₄, filtered, and concentrated in vacuo. The crude material is purified by NP-chromatography to give C-2b.

The following product (Table 7) are available in an analogous manner, but TFA is exchanged with difluoroacetic acid.

TABLE 7

| # | Structure | Tᵣₑₜ [min] | [M + H]⁺ | HPLC method |
|---|---|---|---|---|
| C-2c | 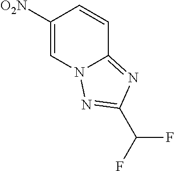 [SMILES: O=[N+]([O-])c1ccc2nc(C(F)F)nn2c1] | 2.5 | 215 | AC |

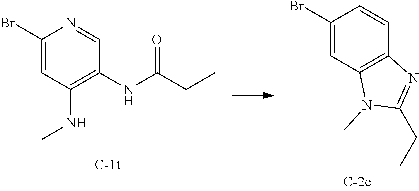

C-1t (30 g, 116 mmol, 1 equiv.) is dissolved in AcOH (150 mL) and heated to 100° C. and stirred for 16 h. Upon complete conversion, the reaction mixture is concentrated, then basified with sodium carbonate to pH 8.5. The aqueous mixture is extracted three times with DCM and the combined organic layers are washed sequentially with ice cold water and brine. The combined organic layers are dried over Na₂SO₄, filtered, and concentrated in vacuo. The crude material is purified by NP chromatography to give C-2e (20 g, 59 mmol; TLC: 10% MeOH in DCM, R_{f}: 0.5; HPLC method AD; tᵣₑₜ=1.1 min; [M+H]+=241).

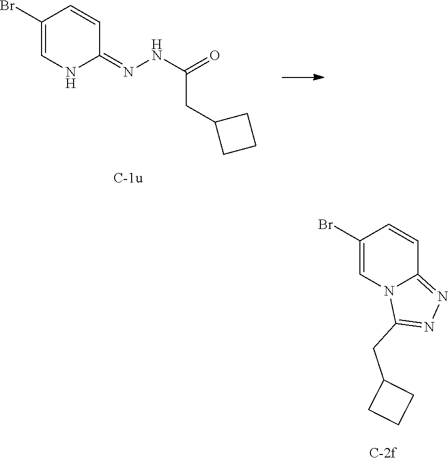 [SMILES: O=C(CC1CCC1)N/N=c1\ccc(Br)c[nH]1 ; Brc1ccc2nnc(CC3CCC3)n2c1]

C-1u (14 g, 74 mmol, 1 equiv.) is dissolved in toluene (140 mL) at 0° C., next pTSA (1.7 g, 9.8 mmol, 0.2 equiv.) is added and the reaction is heated to 120° C. and stirred for 72 h. Upon complete conversion, the reaction is basified with sat. aq. NaHCO₃ and extracted three times with EtOAc. The combined organic layers are dried over Na₂SO₄, filtered, and concentrated in vacuo. The crude material is purified by NP chromatography to give C-2f (8 g, 27 mmol; TLC: 50% EtOAc in Hexane, R_{f}: 0.5; HPLC method AC; tᵣₑₜ=1.4 min; [M+H]+=266).

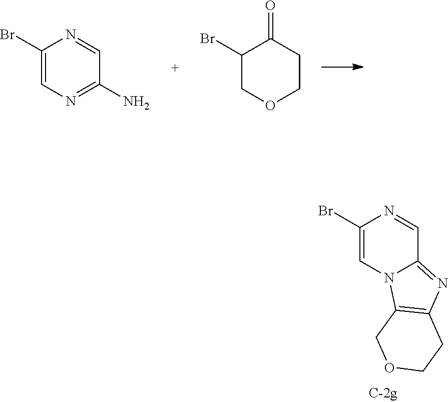 [SMILES: Nc1cnc(Br)cn1 ; O=C1CCOCC1Br ; Brc1cn2c3c(nc2cn1)CCOC3]

3-Bromotetrahydropyran-4-one (10 g, 55.9 mmol, 1 equiv.) and 5-bromopyrazine-2-amine (45.6 g, 84 mmol, 1.5 equiv.) are dissolved in IPA (100 mL) and stirred for 15 minutes. Next, pyridinium p-toluenesulfonate (1.4 g, 5.6 mmol, 0.1 equiv.) is added and the reaction mixture is stirred at 90° C. for 1 h. Upon complete conversion, the reaction mixture is diluted with water and basified with sat. aqueous NaHCO₃ and extracted twice with 10% MeOH in DCM. The combined organic layers are washed with brine and concentrated in vacuo. The crude material is purified by NP chromatography to give C-2g (1.4 g, 8.9 mmol; TLC: 60% EtOAc in PE, R_{f}=0.5; HPLC method AC; tᵣₑₜ=2.3; [M+H]+=254).

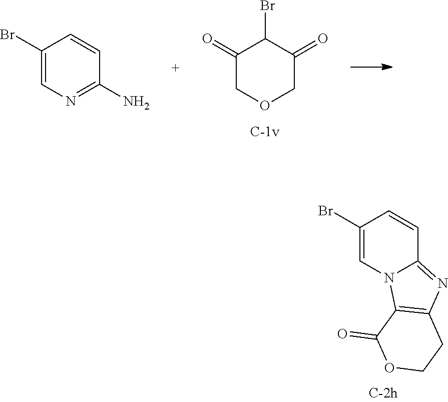 [SMILES: Nc1ccc(Br)cn1 ; O=C1COCC(=O)C1Br ; O=C1OCCc2nc3ccc(Br)cn3c21]

5-Bromopyridin-2-amine (29.6 g, 171 mmol, 1.1 equiv.) is added to a solution of C-1v (30 g, 155 mmol, 1 equiv.) in dimethoxyethane (300 mL) at room temperature. Next, the reaction mixture is heated to 85° C. and stirred for 16 h. Upon complete conversion, the reaction mixture is concentrated in vacuo. The crude material is basified with sat. aqueous NaHCO₃ and extracted with EtOAc. The combined organic layers are dried over Na₂SO₄, filtered, and concentrated in vacuo. The crude material is purified by NP chromatography to give C-2h (6 g, 20 mmol; TLC: 5% DCM in MeOH; R_{f}=0.4; HPLC method AC; tᵣₑₜ=2.5; [M+H]+=267).

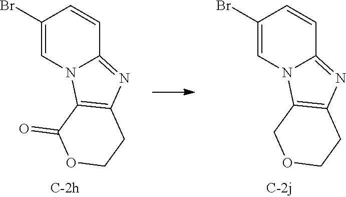 [SMILES: O=C1OCCc2nc3ccc(Br)cn3c21 ; Brc1ccc2nc3c(n2c1)COCC3]

A solution of C-2h (6 g, 22 mmol, 1 equiv.) in THF (30 mL) is cooled to −20° C. and stirred for 15 minutes. Next, borane dimethyl sulfide (3 M, 56 nL, 112 mmol, 5 equiv.) is added dropwise at the same temperature. After complete addition, the reaction mixture is heated to 70° C. and stirred for 4 h. Upon complete conversion, the reaction mixture is cooled to 0° C., quenched with methanol and stirred for a further 1 h. The reaction mixture is concentrated in vacuo and purified by NP chromatography to give C-2j (3.1 g, 3.2 mmol; TLC: 50% EtOAc in PE, R_{f}=0.4; HPLC method AC; tᵣₑₜ=2.4; [M+H]+=253).

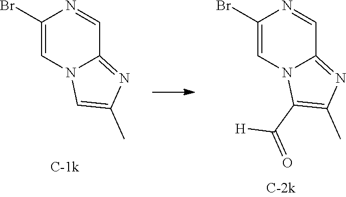

To a solution of POCl₃ (188 g, 588 mmol, 12 equiv.) in DMF (183 mL) at 0° C. is added C-1k (10.2 g, 28 mmol, 1 equiv.) In DMF (204 mL). The reaction mixture is stirred at 85° C. for 8 h. Upon complete conversion, the reaction mixture is quenched with sat. aq NaHCO₃ and extracted three times with Et₂O. The combined organic layers are concentrated in vacuo and purified by NP chromatography to give C-2k (6 g, 23 mmol; TLC: 40% EtOAc in PE, R_{f}=0.6; HPLC method AC; tᵣₑₜ=2.4; [M+H]+=240).

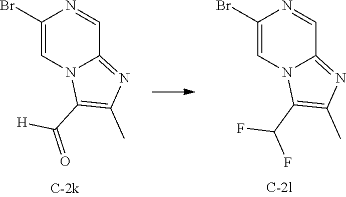

C-2k (6 g, 25 mmol, 1 equiv.) is dissolved in DCM (120 mL), cooled to 0° C. and DAST (12 g, 75 mmol, 3 equiv.) is added. The reaction mixture is then stirred at 40° C. for 32 h. Upon complete conversion, the reaction mixture is quenched with ice cold water and extracted three times with DCM. The combined organic layers are washed with sat. aq NaHCO₃ and concentrated in vacuo. The crude material is purified by NP chromatography to give C-21 (4.2 g, 14 mmol; TLC: 40% EtOAc in Hexane, R_{f}: 0.6; HPLC method AC; tᵣₑₜ=2.5 min; [M+H]+=263).

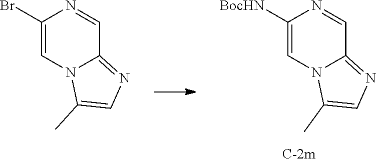 [SMILES: Cc1cnc2cnc(Br)cn12 ; Cc1cnc2cnc(NC(=O)OC(C)(C)C)cn12]

To a stirred solution of 6-bromo-3-methyl-imidazo[1,2-a]pyrazine (22.00 g, 104 mmol, 1.00 equiv) in 1,4-dioxane (330 mL) tert-butyl carbamate (36.42 g, 311 mmol, 3.00 equiv) and cesium carbonate (134.87 g, 415 mmol, 4.00 equiv) are added. Then, nitrogen gas is purged into the reaction for 5-10 min, then finally added Brettphos Pd G3 (5.64 g, 6.22 mmol, 0.06 equiv). The reaction is stirred at 100° C. for 16 hours. Upon completion the reaction is cooled to 0° C. and diluted with ethyl acetate, filtered over celite and washed with water and brine. The combined organic layers is dried with Na₂SO₄, concentrated under reduced pressure and purified with NP chromatography to afford C-2m. (HPLC method D; tᵣₑₜ=2.52 min; [M+H]+=249)

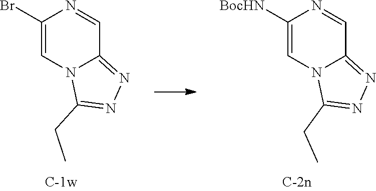 [SMILES: CCc1nnc2cnc(Br)cn12 ; CCc1nnc2cnc(NC(=O)OC(C)(C)C)cn12]

C-1w (10 g, 44 mmol, 1 equiv.) is dissolved in toluene (100 mL), then t-butyl carbamate (15.5 g, 132 mmol, 3 equiv.) and cesium carbonate (57.3 g, 176 mmol, 4 equiv.) are added. The reaction mixture is purged with nitrogen gas for 5-10 minutes, and subsequently BrettPhos Pd G3 (4 g, 4.4 mmol, 0.1 equiv.) is added. The reaction mixture is stirred at 130° C. for 4 h. Upon complete conversion, the reaction mixture is cooled to 0° C., diluted with EtOAc and filtered over a pad of Celite. The organic layers are washed with water and brine, then dried over Na₂SO₄, filtered, and concentrated in vacuo to give the crude solid. The material is purified by NP chromatography to give product C-2n (4.5 g, 16.1 mmol; TLC: 70% EtOAc in Hexane, R_{f}: 0.5; HPLC method AC; tᵣₑₜ=2.5 min, [M+H]+=264).

The following products C-2 (Table 8) are available in an analogous manner. In some cases dioxane (c=0.4 M) is used instead of toluene.

TABLE 8

| | | | | HPLC |
|---|---|---|---|---|
| # | Structure | Tᵣₑₜ [min] | [M + H]⁺ | method |
| C-2n | 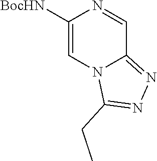 [SMILES: CCc1nnc2cnc(NC(=O)OC(C)(C)C)cn12] | 2.5 | 264 | AC |
| C-2o dioxane | 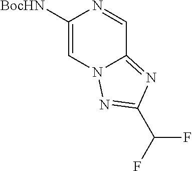 [SMILES: CC(C)(C)OC(=O)Nc1cn2nc(C(F)F)nc2cn1] | 2.7 | 286 | AC |
| C-2p dioxane | 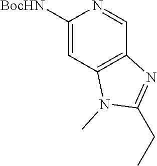 [SMILES: CCc1nc2cnc(NC(=O)OC(C)(C)C)cc2n1C] | 1.1 | 277 | AD |

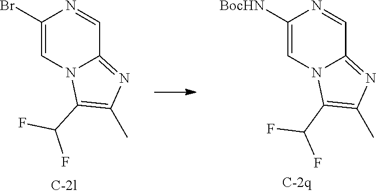 [SMILES: Cc1nc2cnc(Br)cn2c1C(F)F ; Cc1nc2cnc(NC(=O)OC(C)(C)C)cn2c1C(F)F]

C-21 (4.2 g, 61 mmol, 1 equiv.) is dissolved in dioxane (84 mL), then t-butyl carbamate (2.8 g, 24 mmol, 1.5 equiv.), cesium carbonate (13 g, 40 mmol, 1.5 equiv.), and Xantphos (0.93 g, 1.6 mmol, 0.1 equiv.) are added. The reaction mixture is purged with nitrogen gas for 5-10 minutes, and subsequently Pd(OAc)₂ (0.049 mL, 0.48 mmol, 0.03 equiv.) is added. The reaction mixture is stirred at 100° C. for 16 h. Upon complete conversion, the reaction mixture is cooled to 0° C., diluted with EtOAc and filtered over a pad of Celite. The organic layers are washed with water and brine, then dried over Na₂SO₄, filtered, and concentrated in vacuo to give the crude solid. The material is purified by NP chromatography to give product C-2q (3 g, 8.2 mmol; TLC: 5% MeOH in DCM, R_{f}: 0.5; HPLC method AC; tᵣₑₜ=2.6 min, [M+H]+=299).

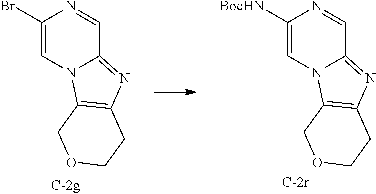 [SMILES: Brc1cn2c3c(nc2cn1)CCOC3 ; CC(C)(C)OC(=O)Nc1cn2c3c(nc2cn1)CCOC3]

C-2g (2.5 g, 9.9 mmol, 1 equiv.) is dissolved in toluene (25 mL) and degassed with nitrogen for 15 minutes. Next, K₂CO₃ (4.1 g, 29 mmol, 3 equiv.) is added and the reaction mixture stirred for 15 minutes continuing to purge with nitrogen. Copper iodide (0.19 g, 0.9 mmol, 0.1 equiv.) and 1,2-dimethylethylenediamine (0.87 g, 9.8 mmol, 1 equiv.) are added and the reaction mixture stirred at 120° C. for 2 h. Upon complete conversion, the reaction mixture is filtered over a pad of Celite® using EtOAc. The filtrate is washed with water, and the combined organic layer dried over Na₂SO₄, filtered and concentrated in vacuo. The crude material is purified by NP chromatography to give C-2r.

The following products C-2 (Table 9) are available in an analogous manner.

TABLE 9

| | | | | HPLC |
|---|---|---|---|---|
| # | Structure | Tᵣₑₜ [min] | [M + H]⁺ | method |
| C-2r | 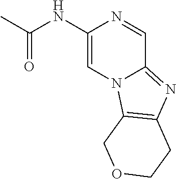 [SMILES: CC(=O)Nc1cn2c3c(nc2cn1)CCOC3] | 2.14 | 233 | AC |
| C-2s | 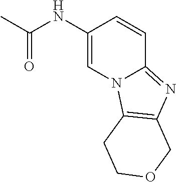 [SMILES: CC(=O)Nc1ccc2nc3c(n2c1)CCOC3] | 2.21 | 232 | AC |

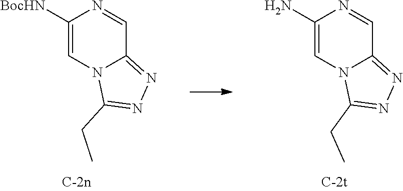 [SMILES: CCc1nnc2cnc(NC(=O)OC(C)(C)C)cn12 ; CCc1nnc2cnc(N)cn12]

C-2n (4.5 g, 17.1 mmol, 1 equiv.) is dissolved in ACN (180 mL) and water (180 mL). The reaction mixture is heated to 110° C. and stirred for 24 h. Upon completion of the reaction, the reaction mixture is concentrated in vacuo and purified by NP-chromatography (using basic silica) to give product C-2t (0.64 g, 3.8 mmol; TLC: 10% MeOH in DCM, R_{f}: 0.5; HPLC method AC; tᵣₑₜ=1.1 min; [M+H]+=164).

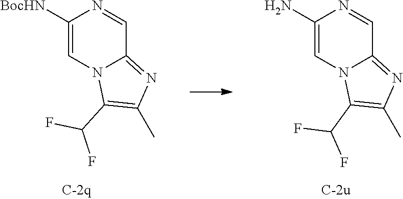 [SMILES: Cc1nc2cnc(NC(=O)OC(C)(C)C)cn2c1C(F)F ; Cc1nc2cnc(N)cn2c1C(F)F]

C-2q (2.8 g, 9.4 mmol, 1 equiv.) is dissolved in DCM (28 mL) and TFA (5.6 mL) at 0° C. The reaction mixture is gradually warmed up and stirred at room temperature for 24 h. Upon completion of the reaction, the reaction mixture is concentrated in vacuo. The crude material is basified with sat. aqueous NaHCO₃ and extracted twice with 10% MeOH in DCM. The combined organic layers are concentrated in vacuo and purified by NP chromatography to give C-2u (291 mg, 1.3 mmol; TLC: 5% MeOH in DCM, R_{f}: 0.5; HPLC method AD; tᵣₑₜ=2.5 min; [M+H]+=186

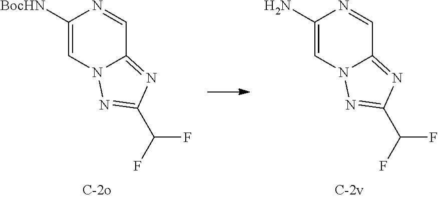 [SMILES: CC(C)(C)OC(=O)Nc1cn2nc(C(F)F)nc2cn1 ; Nc1cn2nc(C(F)F)nc2cn1]

C-2o (13 g, 45.6 mmol, 1 equiv.) is dissolved in HFIP (130 mL) in a steelbomb and the reaction mixture is heated to 100° C. and stirred for 16 h. Upon completion of the reaction, the reaction is concentrated in vacuo and the crude material is washed twice with diethyl ether to give C-2v (2.1 g, 10 mmol; TLC: 50% EtOAc in Hexane, R_{f}: 0.3; HPLC method AD; tᵣₑₜ=2.5 min; [M+H]+=186).

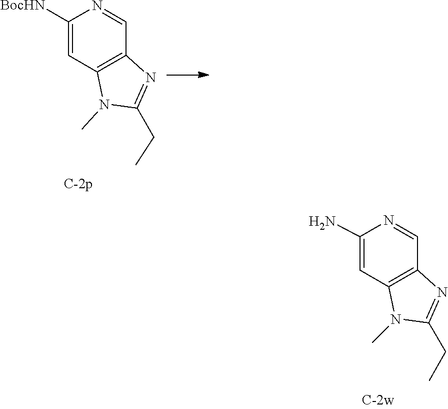 [SMILES: CCc1nc2cnc(NC(=O)OC(C)(C)C)cc2n1C ; CCc1nc2cnc(N)cc2n1C]

C-2p (13 g, 47 mmol, 1 equiv.) is dissolved in 6 N HCl (130 mL) and the reaction mixture is stirred for 16 h. Upon complete conversion, the reaction mixture is basified with sodium carbonate and extracted three times with DCM. The combined organic layers are washed ice cold water and brine, dried over Na₂SO₄, filtered, and concentrated in vacuo. The crude material is purified by NP chromatography to give C-2w (4.7 g, 24.9 mmol; TLC: 10% MeOH in DCM, R_{f}: 0.2; HPLC method AD; tᵣₑₜ=2.1 min; [M+H]+=186).

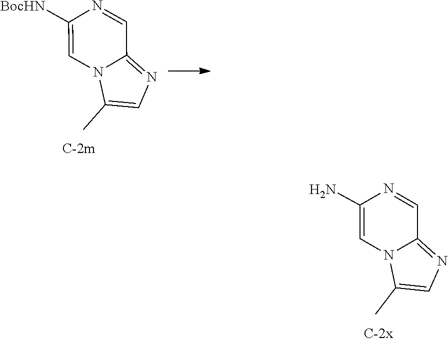 [SMILES: Cc1cnc2cnc(NC(=O)OC(C)(C)C)cn12 ; Cc1cnc2cnc(N)cn12]

To a stirred solution of C-2m (50 mg, 0.20 mmol, 1.00 equiv.) in DCM (3 mL) TFA (0.15 mL, 1.84 mmol, 9.15 equiv.) is added at 0° C. The reaction is stirred at room temperature for 7 h. Upon completion the reaction is concentrated under reduced pressure and diluted with water and ethyl acetate. The aq. layer is basified with sat. NaHCO₃ and extracted with ethyl acetate and dried over Na₂SO₄ to afford C-2x. (HPLC method AC; tᵣₑₜ=2.12 min; [M+H]=149)

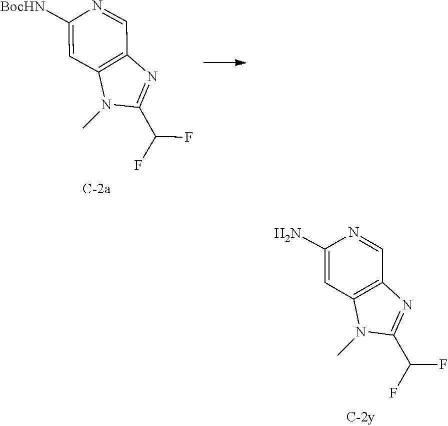 [SMILES: Cn1c(C(F)F)nc2cnc(NC(=O)OC(C)(C)C)cc21 ; Cn1c(C(F)F)nc2cnc(N)cc21]

C-2a (12.5 g, 41.9 mmol, 1 equiv.) is dissolved in DCM (62 mL) and HCl in dioxane (4 M, 126 mL) is added at 0° C. The reaction mixture is stirred at room temperature for 16 h. Upon complete conversion, the reaction mixture is concentrated in vacuo to give C-2y (11 g, 55 mmol; TLC: 10% MeOH in DCM; R_{f}: 0.2; HPLC method AD; tᵣₑₜ=3.5; [M+H]+=199) which is used in the next step without further purification.

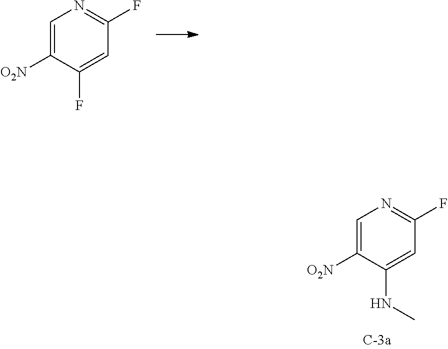

To a solution of methylamine (6.55 g, 15.2 mmol, 1.2 equiv.) and triethylamine (1.41 g, 13.9 mmol, 1.1 equiv.) in THF (5 mL), a solution of 2,4-difluoro-5-nitropyridine (2.11 g, 12.7 mmol, 1.0 equiv.) in THF (5 mL) is added dropwise. The reaction is stirred at room temperature for 40 min. The reaction is extracted with ethylacetate and water. The organic layer is dried over MgSO₄ and evaporated to dryness to afford C-3a (HPLC method C; tᵣₑₜ=0.66; [M+H]+=170), which is used without further purification.

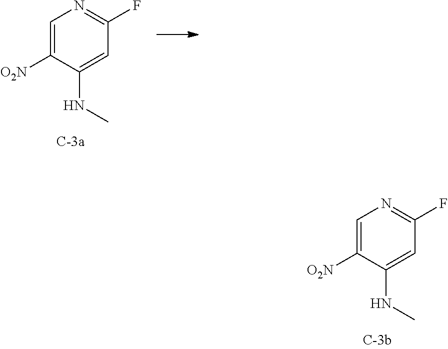

C-3a (2.2 g, 11.6 mmol, 1.0 equiv.) is dissolved in NH₃ (28% in water, 30 mL) and stirred at 60° C. for 2 h. The reaction is dissolved in ethyl acetate/2-propanol and extracted with water. The organic layer is dried over MgSO₄, filtered and evaporated under reduced pressure to afford C-3b (HPLC method C; tᵣₑₜ=0.50-0.54; [M+H]+=169), which is used without further purification.

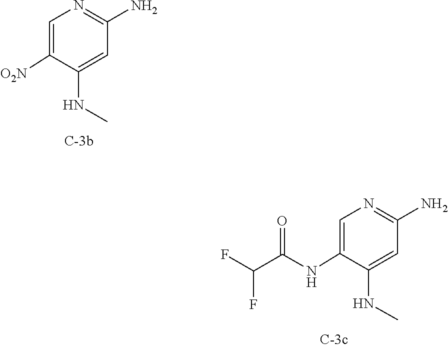

To a solution of C-3b (2.05 g, 12.2 mmol, 1.0 equiv.) in difluoroacetic acid (15 mL) Fe (3.5 g, 5.12 mmol, 5.1 equiv.) is added. The reaction is stirred at 60° C. for 2 h. The reaction is diluted with ethyl acetate and sat. NaHCO₃ is added slowly. The mixture is filtered over celite and then extracted. The organic layer is dried over MgSO₄ and evaporated to dryness to afford C-3c (HPLC method C; tᵣₑₜ=0.14; [M+H]+=217), which is used without further purification.

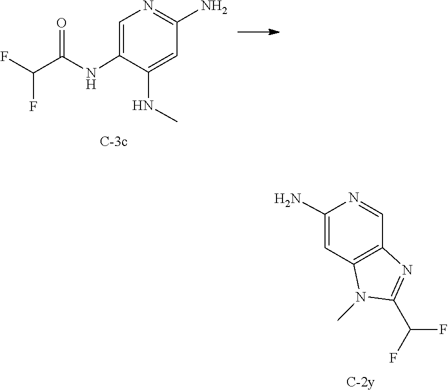

C-3c (3.21 g, 11.9 mmol, 1.0 equiv.) is dissolved in DCE (20 mL) and N,O-bis(trimethylsilyl)acetamide (10.0 mL, 40.8 mmol, 3.4 equiv) is added. The reaction mixture is stirred at 90° C. for 24 h. Upon complete conversion, the reaction mixture is extracted with ethylacetate and sat. NaHCO₃ solution. The organic layer is dried over MgSO₄ and concentrated under reduced pressure, then purified by basic RP chromatography and concentrated in vacuo to give C-2y (HPLC method C; tᵣₑₜ=0.52; [M+H]+=199).

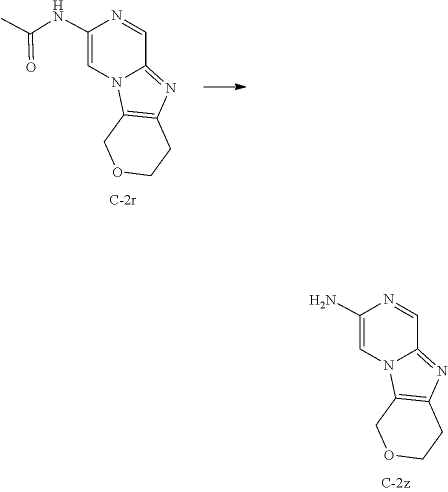 [SMILES: CC(=O)Nc1cn2c3c(nc2cn1)CCOC3 ; Nc1cn2c3c(nc2cn1)CCOC3]

C-2r (2.3 g, 9.9 mmol, 1 equiv.) is dissolved in methanol (25 mL) at 0° C. KOH (1.11 g, 19.8 mmol, 2 equiv.) is added and the reaction mixture stirred at 70° C. for 16 h. Upon complete conversion, the reaction mixture is diluted with water and extracted twice with 10% MeOH/DCM. The combined organic layers are dried over Na₂SO₄, filtered, and concentrated in vacuo. The crude material is washed three times with Et₂O to give product C-2z (0.77 g, 3.6 mmol; TLC: 10% MeOH in DCM, R_{f}: 0.2; HPLC method AD; tᵣₑₜ=4.9 min, [M+H]+=191).

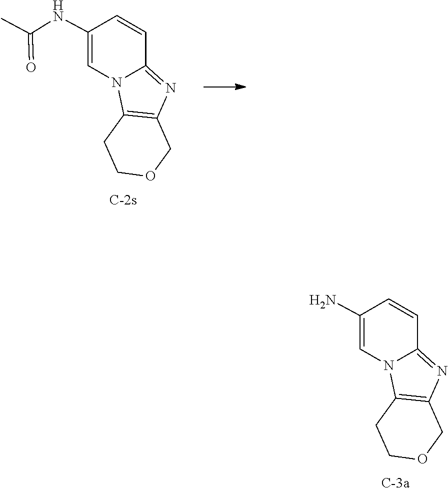 [SMILES: CC(=O)Nc1ccc2nc3c(n2c1)CCOC3 ; Nc1ccc2nc3c(n2c1)CCOC3]

C-2s (1.6 g, 6.9 mmol, 1 equiv.) and SOCl₂ (3.3 g, 28 mmol, 4 equiv.) are dissolved in methanol (80 mL) and the reaction mixture is stirred at room temperature for 5 h. Upon complete conversion, the reaction is concentrated in vacuo. The crude material is basified with sat. aqueous sodium hydrogen carbonate and extracted with 5% MeOH/DCM. The combined organic layers are washed with brine and concentrated in vacuo. The crude material is purified by NP chromatography to give C-3a (0.13 g, 0.6 mmol; TLC: 10% MeOH in DCM, R_{f}: 0.5; HPLC method AD; tᵣₑₜ=2.3 min, [M+H]+=190).

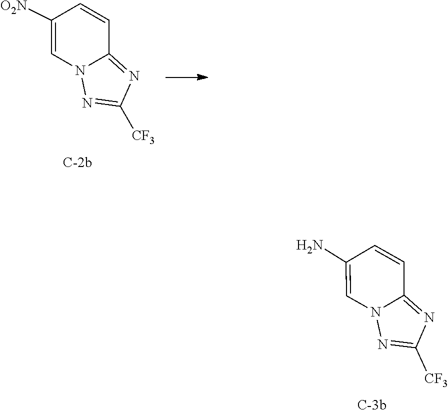 [SMILES: O=[N+]([O-])c1ccc2nc(C(F)(F)F)nn2c1 ; Nc1ccc2nc(C(F)(F)F)nn2c1]

C-2b (0.5 g, 2.1 mmol, 1 equiv.) and iron powder (360 mg, 6.5 mmol, 3 equiv.) in acetic acid (2.5 mL) is heated to 50° C. and stirred for 5 h. Upon completion of the reaction, the reaction mixture is cooled and filtered over a pad of Celite® using DCM. The filtrate is washed with water, and the combined organic layer dried over Na₂SO₄, filtered and concentrated in vacuo. The crude material is washed twice with pentane and dried under vacuum to give C-3b HPLC method AD; tᵣₑₜ=1.4 min, [M+H]+=203) The following products C-3 (Table 10) are available in an analogous manner.

TABLE 10

| | | | | HPLC |
|---|---|---|---|---|
| # | Structure | Tᵣₑₜ [min] | [M + H]⁺ | method |
| C-3c | 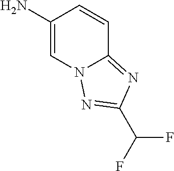 [SMILES: Nc1ccc2nc(C(F)F)nn2c1] | 1.1 | 185 | AD |

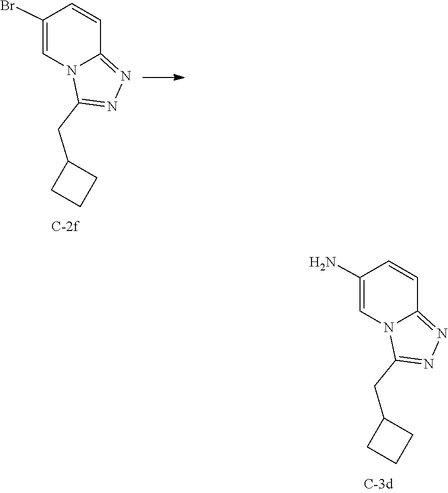 [SMILES: Brc1ccc2nnc(CC3CCC3)n2c1 ; Nc1ccc2nnc(CC3CCC3)n2c1]

C-2f (6 g, 22.5 mmol, 1 equiv.), diphenylmethanimine (6.13 g, 33.8 mmol, 1.5 equiv.), sodium t-butoxide (4.3 g, 45 mmol, 2 equiv.) and Xanthphos (1.3 g, 2.2 mmol, 0.1 equiv.) in dioxane (60 mL) is degassed for 5 minutes with nitrogen. Next, Pd(dba)₃ (1 g, 1.1 mmol, 0.05 equiv.) is added and the reaction mixture is stirred at 100° C. for 12 h. Upon complete conversion, the reaction mixture is filtered over a pad of Celite® and washed with 5% MeOH in DCM (50 mL). The filtrate is concentrated in vacuo and the crude material is purified by NP chromatography to give C-3d (1 g, 4.6 mmol; TLC: 10% MeOH in DCM, R_{f}: 0.2; HPLC method AC; tᵣₑₜ=1.2 min, [M+H]+=203).

### Synthesis of F-1

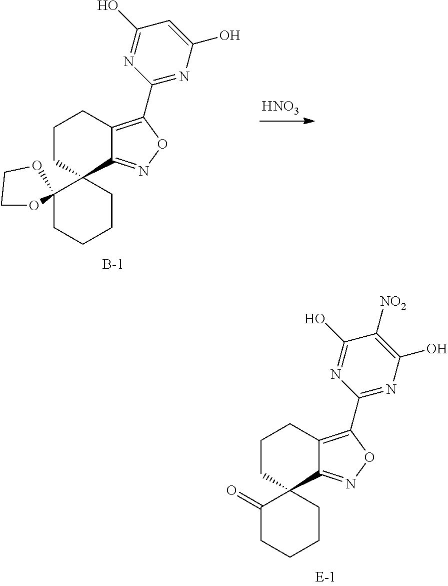

A solution of B-1 (WO2024115529, 130 g, 362 mmol, 1.0 equiv.) in glacial acetic acid (650 mL) is stirred at 0° C. To this, fuming nitric acid (65 mL, 362 mmol, 1.0 equiv.) is added. The resulting mixture is allowed to warm to room temperature and stirred for 2 hours. The progress of the reaction is monitored by TLC. Upon completion, the reaction is quenched with ice water and stirred for 30 minutes. The solid product is collected by filtration, then slurried in methyl tert-butyl ether, stirred for 30 minutes, filtered and dried. The solid is then co-evaporated with toluene under reduced pressure to give the product, E-1, as a pale yellow solid (HPLC method X, tᵣₑₜ=1.22 min; [M+H]=361).

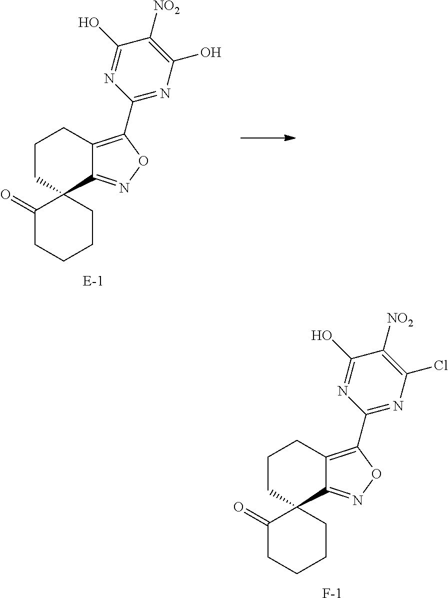

A solution of E-1 (120 g, 333 mmol, 1.0 equiv.) in toluene (1200 mL) is stirred at room temperature. To this, N,N-Diisopropylethylamine (290 mL, 1665 mmol, 5.0 equiv.) is added, followed by a dropwise addition of phosphoryl chloride (155 mL, 1665 mmol, 5.0 equiv.) at 0° C. The resulting reaction mixture is stirred at 0° C. to room temperature for 3 hours. The progress of the reaction is monitored by TLC. Upon completion, the reaction is quenched with ice water and stirred for 10 minutes. The mixture is then extracted with methyl tert-butyl ether. The organic layer is washed with aqueous sodium bicarbonate and brine, dried over anhydrous sodium sulfate, filtered and concentrated under reduced pressure to give the crude product as a brown solid. The crude product is purified by flash column chromatography, eluting with 15% ethyl acetate in hexane. The desired fractions are concentrated in vacuo to give the product, F-1, as a solid (HPLC method AD, tᵣₑₜ=01.24 min; [M+H]=397).

### Overview Scheme to O-1a and O-1b

### O-1c and O-1d are Prepared Analogously

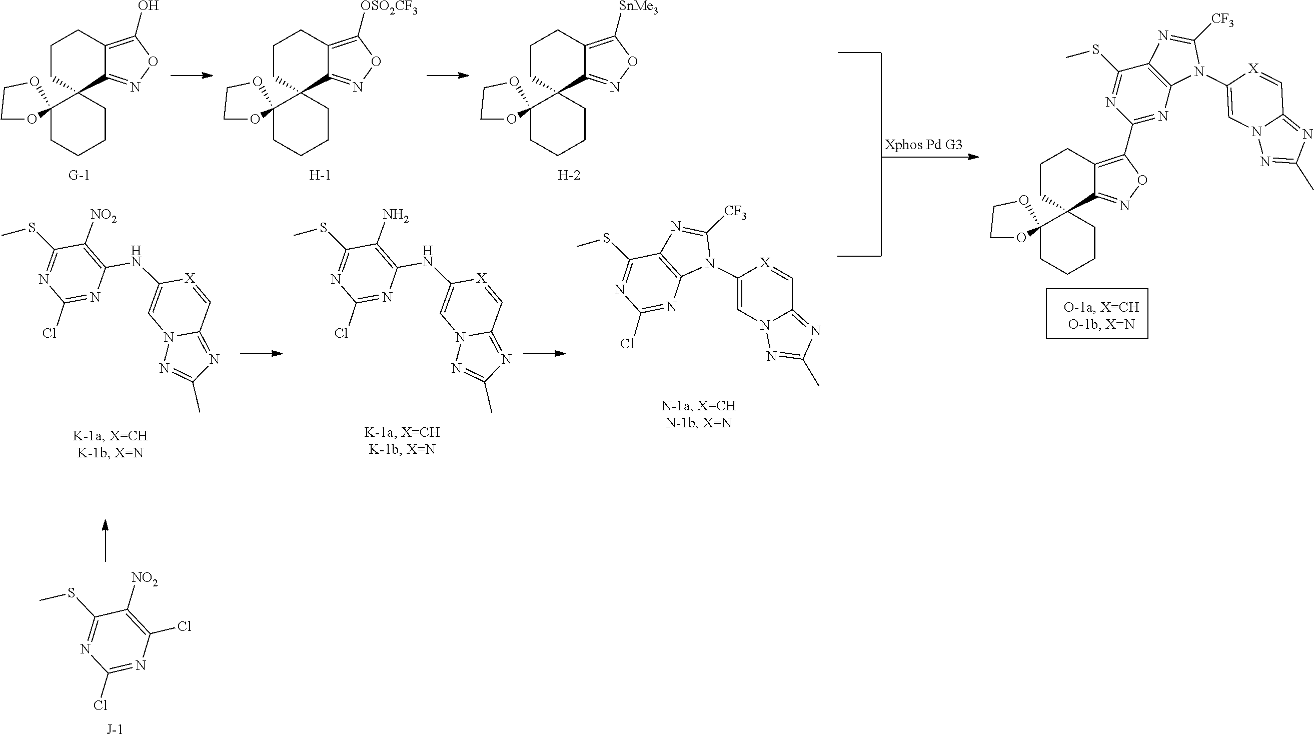

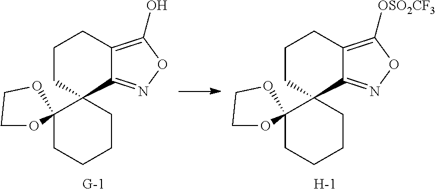

A solution of G-1 (10 g, 37.7 mmol, 1.0 equiv.) in dichloromethane (200 mL) is stirred at room temperature. To this, triethylamine (2.0 equiv., 7.61 g, 75.4 mmol) is added. The reaction mixture is then cooled to −78° C. Triflic anhydride (1.5 equiv., 15.94 g, 56.5 mmol) is added dropwise to the cooled mixture. The resulting mixture is allowed to stir at −78° C. for 4 hours. The reaction is monitored by LCMS. Upon completion, ice-cold water is added to the reaction mixture. The organic layer is separated and washed with water. The organics are then dried with sodium sulfate (Na₂SO₄) and concentrated to dryness. The crude product is purified by flash column chromatography, eluting with 10% ethyl acetate in hexane. The desired fractions are concentrated in vacuo to afford the product H-1.

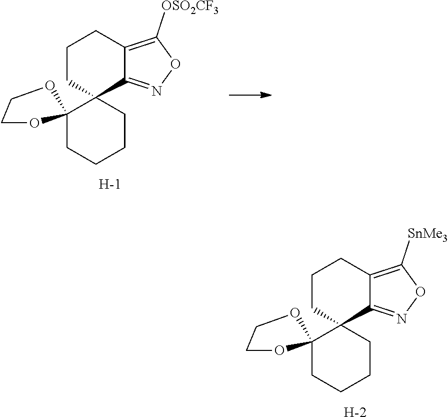

A solution of H-1 (40 g, 101 mmol, 1.0 equiv.) in 1,4-dioxane (400 mL) is degassed with nitrogen for 15-20 minutes. To this, lithium chloride (3.0 equiv., 12.68 g, 302 mmol) and hexamethylditin (1.3 equiv., 42.86 g, 131 mmol) are added. The reaction mixture is stirred at room temperature under a nitrogen atmosphere for 15 minutes. Tetrakis(triphenylphosphine)palladium(0) (0.08 equiv., 9.305 g, 8.05 mmol) is then added. The reaction mixture is heated to 100° C. and maintained at this temperature for 3 hours. Upon completion, the reaction mixture is filtered through a celite pad and washed with a 10% solution of ethyl acetate in petroleum ether. The filtrate is then concentrated under reduced pressure to afford the crude product. The crude product is purified by reverse phase column chromatography to give the desired product H-2 (HPLC method AF, tᵣₑₜ=5.18 min; [M+H]=414).

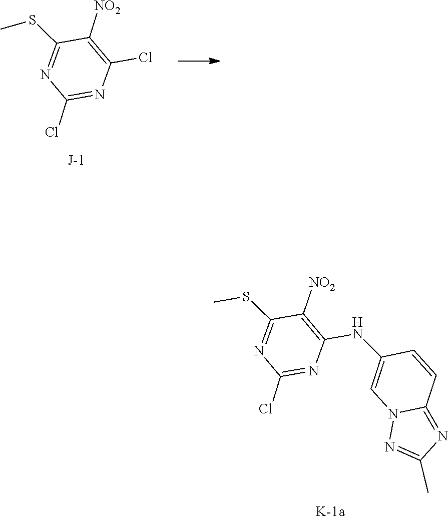 [SMILES: CSc1nc(Cl)nc(Cl)c1[N+](=O)[O-] ; CSc1nc(Cl)nc(Nc2ccc3nc(C)nn3c2)c1[N+](=O)[O-]]

A solution of 2-methyl-[1,2,4]triazolo[1,5-a]pyridin-6-amine (50 g, 337 mmol, 1.0 equiv.) in acetonitrile (250 mL) is stirred at room temperature. To this, DIPEA (2.0 equiv., 87.22 g, 675 mmol) is added and the mixture is stirred for 30 minutes. 2,4-dichloro-6-methylsulfanyl-5-nitro-pyrimidine J-1 (1.1 equiv., 89.11 g, 371 mmol) is then added and the reaction is stirred for an additional 30 minutes. Upon completion, the reaction mixture is diluted with ice-cold water and filtered. The solid is washed with ice-cold water and dried under vacuum to afford the product, K-1a, as a light yellow solid (H PLC method AD, tᵣₑₜ=1.07 min; [M+H]=352).

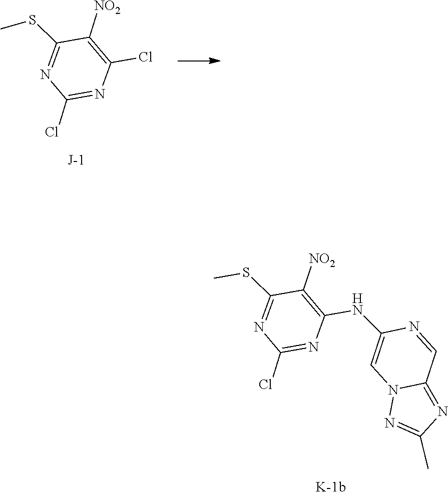 [SMILES: CSc1nc(Cl)nc(Cl)c1[N+](=O)[O-] ; CSc1nc(Cl)nc(Nc2cn3nc(C)nc3cn2)c1[N+](=O)[O-]]

A solution of 2-methyl-[1,2,4]triazolo[1,5-a]pyrazin-6-amine (3 g, 20.1 mmol, 1.0 equiv.) in acetonitrile (45 mL) is stirred at room temperature. To this, DIPEA (7 mL, 40.2 mmol, 2.0 equiv.) is added and the mixture is stirred at room temperature for 1 hour. The reaction mixture is then cooled to 0° C. and 2,4-dichloro-6-methylsulfanyl-5-nitro-pyrimidine J-1 (5.31 g, 22.1 mmol, 1.1 equiv.) is added. The reaction mixture is stirred at room temperature for 4 hours. The progress of the reaction is monitored by TLC. Upon completion, the reaction mixture is concentrated and water is added to the residue. A solid precipitates, which is filtered and dried under vacuum to give the crude product, K-1b, as a solid (HPLC method AF, tᵣₑₜ=4.65 min; [M+H]=352).

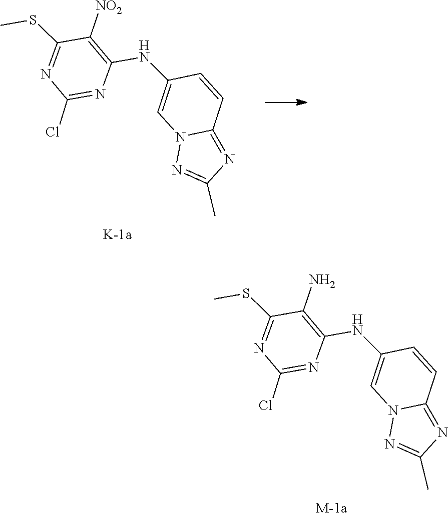 [SMILES: CSc1nc(Cl)nc(Nc2ccc3nc(C)nn3c2)c1[N+](=O)[O-] ; CSc1nc(Cl)nc(Nc2ccc3nc(C)nn3c2)c1N]

A solution of N-(2-chloro-6-methylsulfanyl-5-nitro-pyrimidin-4-yl)-2-methyl-[1,2,4]triazolo[1,5-a]pyridin-6-amine K-1a (350 g, 995 mmol, 1.0 equiv.) in ethanol (2800 mL) and water (700 mL) is stirred. To this, iron powder (3.0 equiv., 166.71 g, 2985 mmol) and ammonium chloride (3.0 equiv., 92.53 g, 2985 mmol) are added. The mixture is then stirred at 90° C. for 16 hours. The progress of the reaction is monitored by TLC and LCMS. Upon completion, the reaction mixture is concentrated under vacuum and passed through a Celite bed, washing with ethyl acetate (500 mL). The residue is diluted with water (300 mL) and extracted with ethyl acetate (2×250 mL). The combined organic layers are washed with water (200 mL) and brine (200 mL), dried over Na2SO4, and the solvent is evaporated to give the crude product. The crude product is passed through basic silica gel (60-100 mesh) and eluted with a 5% methanol/DCM mixture to give the product M-1a (HPLC method AD, tᵣₑₜ=2.53 min; [M+H]=322).

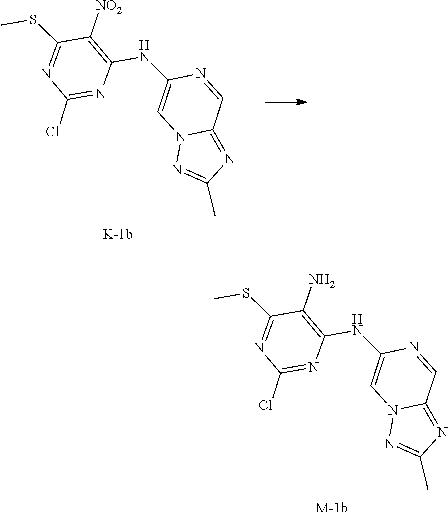 [SMILES: CSc1nc(Cl)nc(Nc2cn3nc(C)nc3cn2)c1[N+](=O)[O-] ; CSc1nc(Cl)nc(Nc2cn3nc(C)nc3cn2)c1N]

A solution of N-(2-chloro-6-methylsulfanyl-5-nitro-pyrimidin-4-yl)-2-methyl-[1,2,4]triazolo[1,5-a]pyrazin-6-amine K-1b (4.9 g, 13.9 mmol, 1.0 equiv.) in ethanol (39.2 mL) and water (9.8 mL) is stirred at room temperature. To this, iron (3.88 g, 69.5 mmol, 5.0 equiv.) and ammonium chloride (3.75 g, 69.5 mmol, 5.0 equiv.) are added. The reaction mixture is stirred at 80° C. for 16 hours. The progress of the reaction is monitored by TLC (60% ethyl acetate in petroleum ether). Upon completion, the reaction mixture is concentrated under vacuum to give a residue. The residue is dissolved in 10% methanol in dichloromethane and filtered through a Celite pad. The filtrate is concentrated under vacuum to give the crude product, M-1b, as a solid (HPLC method AF, tᵣₑₜ=4.27 min; [M+H]=323).

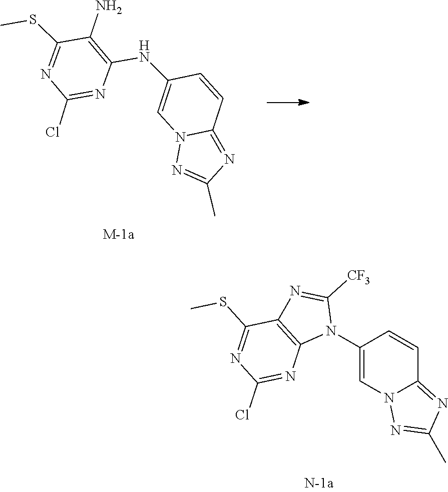

A solution of 2-chloro-6-methylsulfanyl-N4-(2-methyl-[1,2,4]triazolo[1,5-a]pyridin-6-yl)pyrimidine-4,5-diamine M-1a (283 g, 879 mmol, 1.0 equiv.) in 1,2-dichloroethane (2264 mL) is stirred at room temperature. To this, triethylamine (3.0 equiv., 267 g, 2640 mmol) is added, followed by the addition of trifluoroacetic anhydride (2.0 equiv., 369.4 g, 1760 mmol) in 1,2-dichloroethane (476 mL). The reaction mixture is stirred at room temperature for 2 hours. The progress of the reaction is monitored by TLC. Upon completion, the reaction mixture is concentrated under vacuum. The residue is diluted with cold water (500 mL) and extracted with ethyl acetate (2×500 mL). The combined organic layers are washed with water (500 mL) and brine (500 mL), dried over sodium sulfate, and the solvent is evaporated to give the crude product. The crude product is purified by flash column chromatography using 100-200 mesh silica gel and 30% ethyl acetate in petroleum ether as the eluent to give the product N-1a (HPLC method AD, tᵣₑₜ=2.72 min; [M+H]=400).

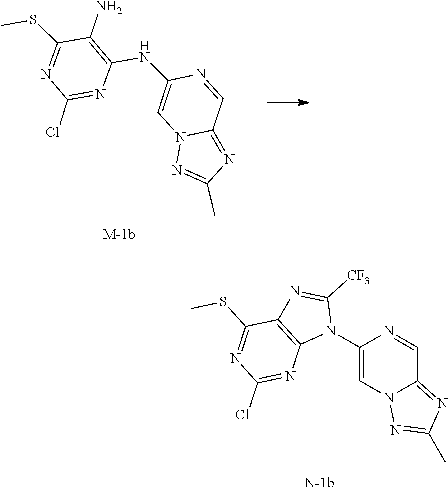

A solution of 2-chloro-6-methylsulfanyl-N4-(2-methyl-[1,2,4]triazolo[1,5-a]pyrazin-6-yl)pyrimidine-4,5-diamine M-1b (4 g, 12.4 mmol, 1.0 equiv.) in toluene (60 mL) is stirred at 0° C. To this, p-toluenesulfonic acid (1.18 g, 6.2 mmol, 0.5 equiv.) and trifluoroacetic anhydride (8 mL, 57.1 mmol, 4.61 equiv.) are added. The reaction mixture is stirred at 120° C. for 36 hours. The progress of the reaction is monitored by TLC. Upon completion, the reaction mixture is concentrated under vacuum to give a residue. The residue is basified with aqueous sodium bicarbonate solution and extracted with ethyl acetate. The organic layer is dried over sodium sulfate and concentrated under vacuum to give the crude product. The crude product is purified by flash column chromatography, eluting with 30% ethyl acetate in petroleum ether, to give the pure product N-1b as a solid (HPLC method AF, tᵣₑₜ=4.64 min; [M+H]=401).

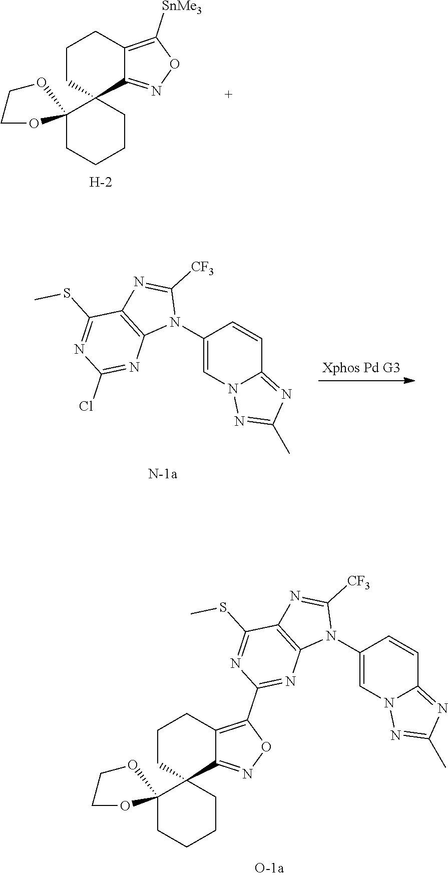

A solution of N-1a (32 g, 80 mmol, 1.0 equiv.) and H-2 (49.48 g, 120 mmol, 1.5 equiv.) in 1,4-dioxane (350 mL) is degassed under a nitrogen atmosphere for 10 minutes. To this, Xphos Pd G3 (6.78 g, 8 mmol, 0.1 equiv.) is added at room temperature. The mixture is then degassed under a nitrogen atmosphere for another 10 minutes and stirred at 120° C. in a microwave for 30 minutes. The progress of the reaction is monitored using TLC (70% ethyl acetate/hexane, Rf=0.5). Upon completion, the reaction mixture is filtered through Celite and the solvent is evaporated under vacuum to give the crude product. The crude product is purified by flash column chromatography (70% ethyl acetate/hexane) to give the product O-1a, as an off-white solid (HPLC method A, tᵣₑₜ=2.32 min; [M+H]=613).

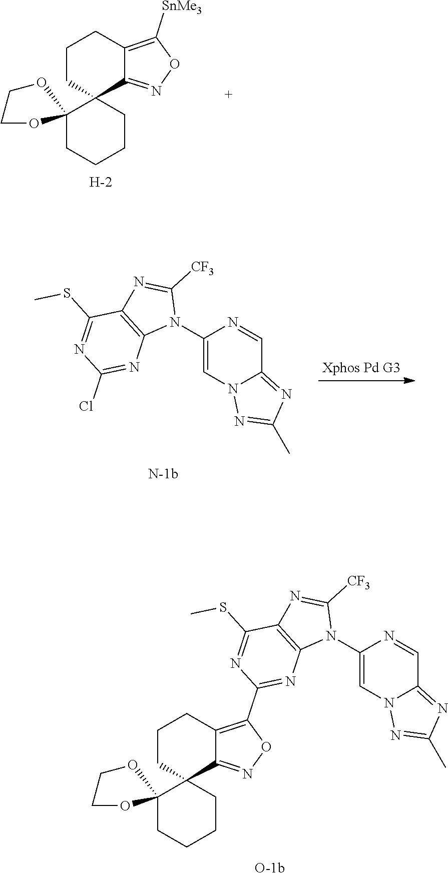

A solution of 2-chloro-6-methylsulfanyl-9-(2-methyl-[1,2,4]triazolo[1,5-a]pyrazin-6-yl)-8-(trifluoromethyl)purine N-1b (0.95 g, 2.37 mmol, 1.0 equiv.) in dioxane (15 mL) is stirred at room temperature. To this, H-2 (1.07 g, 2.61 mmol, 1.1 equiv.) is added. The reaction mixture is degassed with argon for 5 minutes. Then, XPhos Pd G3 (0.20 g, 0.237 mmol, 0.1 equiv.) is added. The reaction mixture is stirred at 120° C. for 2 hours. The progress of the reaction is monitored by TLC (50% ethyl acetate in petroleum ether). Upon completion, the reaction mixture is concentrated under vacuum to give the crude product. The crude product is purified by flash column chromatography, eluting with 30% ethyl acetate in petroleum ether, to give the pure product O-1b (HPLC method AF, tᵣₑₜ=4.27 min; [M+H]=614).

The following products O-1c and O-1d are available in an analogous manner from J-1 and C-2x and [1,2,4]Triazolo[1,5-a]pyrazin-6-amine respectively.

The following intermediate compounds (Table 11) are obtained analogously.

TABLE 11

| | | tᵣₑₜ | | |
|---|---|---|---|---|
| # | Structure | [min] | [M + H]⁺ | HPLC method |
| O-1c | 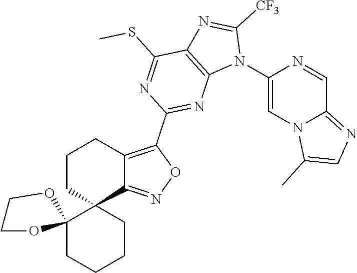 | 3.50 | 613 | AH |
| O-1d | 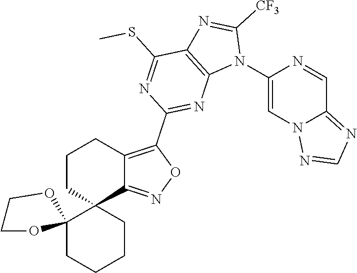 | 5.04 | 599 | Y |

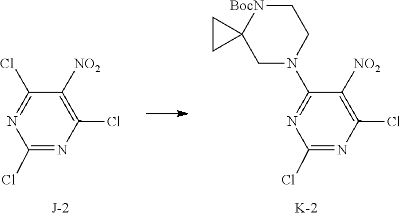

A solution of 2,4,6-trichloro-5-nitro-pyrimidine J-2 (10 g, 43.8 mmol, 1.0 equiv.) in DCM (100 mL) is stirred at room temperature. To this, N-Ethyldiisopropylamine (48.11 g, 52.5 mmol, 1.20 equiv.) and tert-butyl 4,7-diazaspiro[2.5]octane-4-carboxylate (9.29 g, 43.8 mmol, 1.0 equiv.) are added. The reaction mixture is stirred at room temperature for 1 hour.

After the reaction is complete, the reaction mixture is extracted with DCM (2×100 mL), dried over sodium sulfate, and concentrated under vacuum to afford the crude product. The crude compound is purified by column chromatography on silica gel, eluting the desired compound K-2 (HPLC method A, t_{ref}=1.60 min; [M+H]=404).

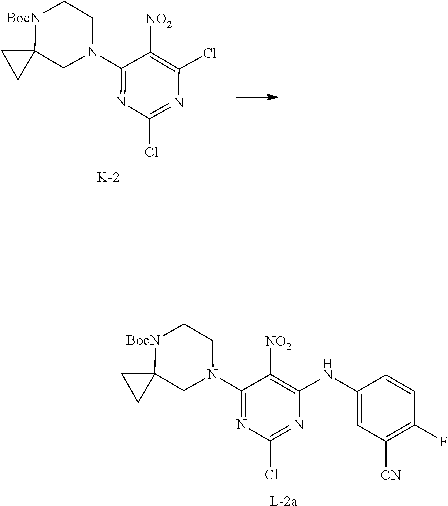

A solution of K-2 (242 mg, 0.60 mmol, 1.0 equiv.) and 5-amino-2-fluorobenzonitrile (84 mg, 0.60 mmol, 1.0 equiv.) in DCM (5 mL) is treated with DIPEA (0.093 g, 0.72 mmol, 1.2 equiv.) and stirred at room temperature for 5 days.

The reaction mixture is then diluted with DCM and extracted with an ammonium chloride solution. The organic phase is dried with magnesium sulfate, filtered, and the solvents are removed under reduced pressure to afford the product L-2a (HPLC method A, tᵣₑₜ=1.35 min; [M+H]=504).

The following products L-2 (Table 12) are available in an analogous manner.

TABLE 12

| | | tᵣₑₜ | | |
|---|---|---|---|---|
| # | Structure | [min] | [M + H]⁺ | HPLC method |
| L-2b | 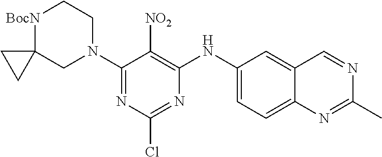 [SMILES: Cc1ncc2cc(Nc3nc(Cl)nc(N4CCN(C(=O)OC(C)(C)C)C5(CC5)C4)c3[N+](=O)[O-])ccc2n1] | 1.05 | 527 | A |

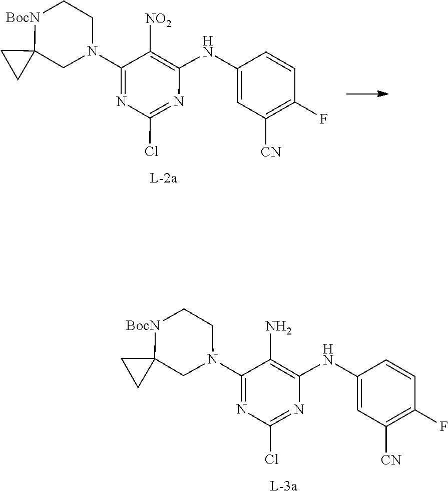

To a suspension of L-2a (100 mg, 0.18 mmol, 1.0 equiv.) in THF (20 mL), sponge nickel catalyst (50% aq. slurry, 21 mg, 0.18 mmol, 1.0 equiv.) is added. The reaction is flushed 3 times with N₂, then filled with 3 bar H₂ and stirred for 4 h at room temperature. The reaction is filtered over celite and concentrated under reduced pressure to afford L-3a without further purification. (HPLC method B, t_{ref}=0.92 min; [M+H]=474).

The following products L-2 (Table 13) are available in an analogous manner.

TABLE 13

| | | tᵣₑₜ | | |
|---|---|---|---|---|
| # | Structure | [min] | [M + H]⁺ | HPLC method |
| L-3b | 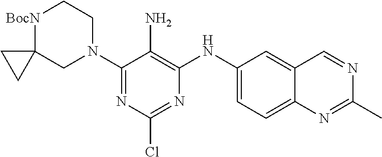 [SMILES: Cc1ncc2cc(Nc3nc(Cl)nc(N4CCN(C(=O)OC(C)(C)C)C5(CC5)C4)c3N)ccc2n1] | 1.02 | 497 | A |

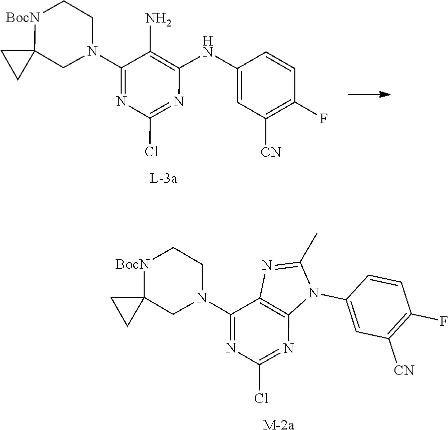

A solution of L-3a (128 mg, 0.27 mmol, 1.0 equiv.) in acetic acid (1 mL) is treated with trimethyl orthoacetate (164 mg, 1.35 mmol, 5.0 equiv.) and stirred at room temperature for 18 hours.

The reaction mixture is then diluted with DCM and extracted with a potassium carbonate solution. The organic phase is dried with magnesium sulfate, filtered, and the solvents are removed under reduced pressure to afford the crude product.

The crude product is dissolved in DMSO, filtered, and purified by preparative HPLC-MS to give M-2a (HPLC method A, tᵣₑₜ=1.55 min; [M+H]=498).

The following products M-2 (Table 14) are available in an analogous manner.

TABLE 14

| # | Structure | tᵣₑₜ [min] | [M + H]⁺ | HPLC method |
|---|---|---|---|---|
| M-2b | 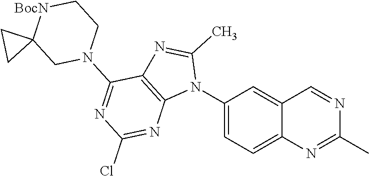 [SMILES: Cc1ncc2cc(-n3c(C)nc4c(N5CCN(C(=O)OC(C)(C)C)C6(CC6)C5)nc(Cl)nc43)ccc2n1] | 1.51 | 522 | A |

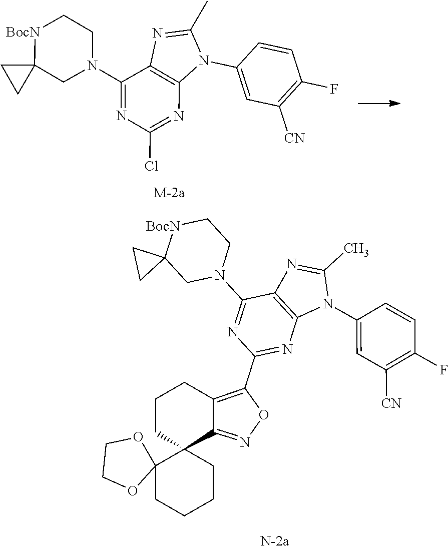

A solution of M-2a (130 mg, 0.261 mmol, 1.0 equiv.), H-2 (118 mg, 0.287 mmol, 1.1 equiv.), and XPhos Pd G3 (22.1 mg, 0.0261 mmol, 0.1 equiv.) in dioxane (2.5 mL) is degassed with argon and heated in a microwave at 120° C. for 15 minutes.

The reaction mixture is then concentrated under reduced pressure, dissolved in DCM, filtered, and purified by normal phase chromatography. The product fractions are then concentrated under reduced pressure to afford N-2a (HPLC method A, tᵣₑₜ=1.63 min; [M+H]=711)

The following products N-2 (Table 15) are available in an analogous manner.

TABLE 15

| # | Structure | tᵣₑₜ [min] | [M + H]⁺ | HPLC method |
|---|---|---|---|---|
| N-2b | 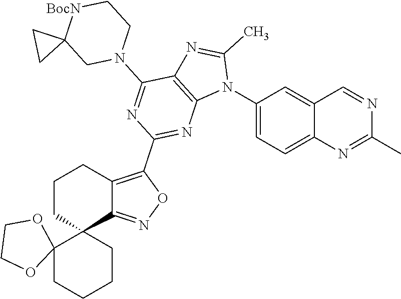 [SMILES: Cc1ncc2cc(-n3c(C)nc4c(N5CCN(C(=O)OC(C)(C)C)C6(CC6)C5)nc(-c5onc6c5CCC[C@@]65CCCCC56OCCO6)nc43)ccc2n1] | 1.78 | 743 | A |

### Coupling of Amines and Building Blocks O-1a/O-1b to P-1

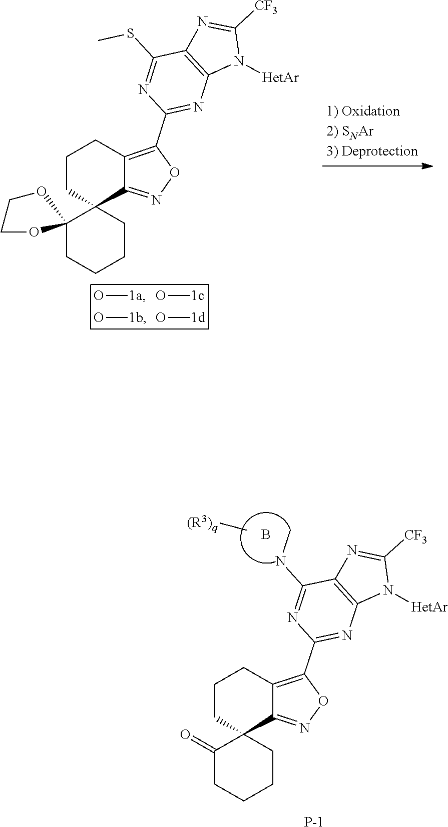

[6-methylsulfanyl-9-(2-methyl-[1,2,4]triazolo[1,5-a]pyridin-6-yl)-8-(trifluoromethyl)purin-2-yl]dispiro (X═CH) O-1a (539.1 mg, 0.880 mmol, 1.0 equiv) is dissolved in CH₂Cl₂ (5 mL), 3-chloroperbenzoic acid (334.1 mg, 1.94 mmol, 2.2 equiv) was added and the reaction mixture was stirred at room temperature for 5 h. After addition of water (20 mL), the phases were separated, and the aqueous phase was extracted with CH₂Cl₂ three times. The combined extracts were dried with Na₂SO₄ and evaporated to dryness. The crude product was used without further purification in the S_{N}Ar step.

The corresponding amine (1.5 equiv) and the crude product from above were dissolved in acetonitrile (0.1 M), cesium carbonate (2.0 equiv) was added and the reaction mixture was stirred at 90° C. for 16 h. After addition of water, the phases were separated, and the aqueous phase was extracted with CH₂Cl₂ three times. The combined extracts were dried with Na₂SO₄ and evaporated to dryness. The crude product was used without further purification in the deprotection step.

The crude product from above was dissolved in dioxane (0.2 M) and 6 M hydrochloric acid (16 equiv) was added. The reaction mixture was stirred at 50° C. for 1 hour. After addition of water, the phases were separated, and the aqueous phase was extracted with CH₂Cl₂ three times. The combined extracts were dried with Na₂SO₄ and evaporated to dryness. The crude product was purified by RP-chromatography to give the desired products P-1.

The following products P-1 (Table 16) are available in an analogous manner.

TABLE 16

| # | Structure | tᵣₑₜ [min] | [M + H]⁺ | HPLC method |
|---|---|---|---|---|
| P-1a | 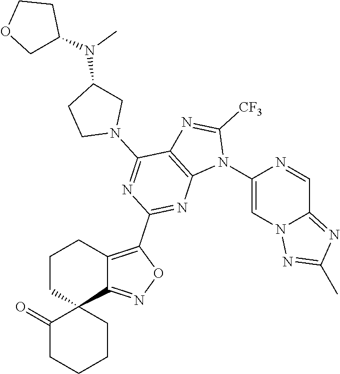 [SMILES: Cc1nc2cnc(-n3c(C(F)(F)F)nc4c(N5CC[C@H](N(C)[C@H]6CCOC6)C5)nc(-c5onc6c5CCC[C@@]65CCCCC5=O)nc43)cn2n1] | 1.07 | 692 | A |
| P-1b | 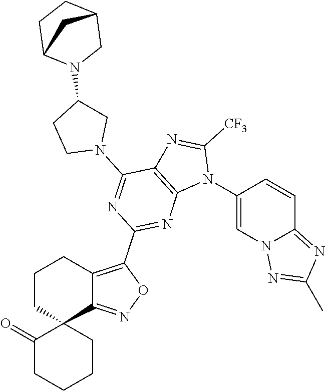 [SMILES: Cc1nc2ccc(-n3c(C(F)(F)F)nc4c(N5CC[C@H](N6C[C@H]7CC[C@@H]6C7)C5)nc(-c5onc6c5CCC[C@@]65CCCCC5=O)nc43)cn2n1] | 1.13 | 687 | A |
| P1-c | 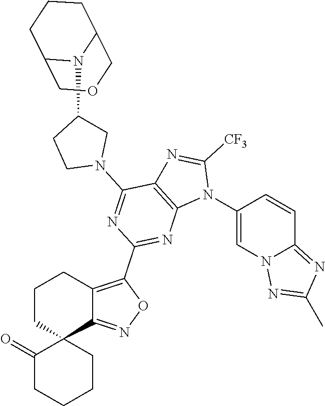 [SMILES: Cc1nc2ccc(-n3c(C(F)(F)F)nc4c(N5CC[C@H](N6C7CCCC6COC7)C5)nc(-c5onc6c5CCC[C@@]65CCCCC5=O)nc43)cn2n1] | 1.03 | 702 | A |
| P-1d | 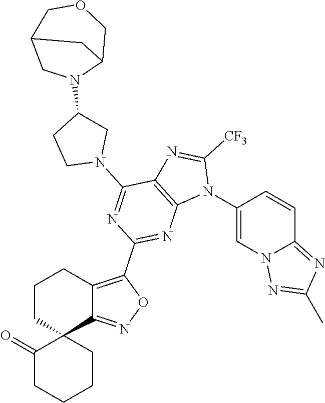 [SMILES: Cc1nc2ccc(-n3c(C(F)(F)F)nc4c(N5CC[C@H](N6CC7COCC6C7)C5)nc(-c5onc6c5CCC[C@@]65CCCCC5=O)nc43)cn2n1] | 1.09 | 702 | A |
| P-1e | 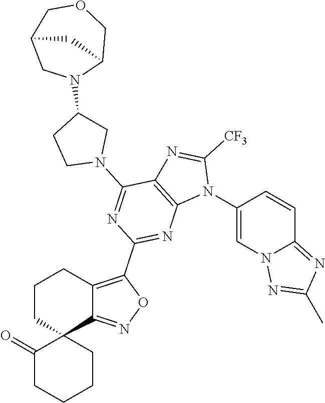 [SMILES: Cc1nc2ccc(-n3c(C(F)(F)F)nc4c(N5CC[C@H](N6C[C@H]7COC[C@@H]6C7)C5)nc(-c5onc6c5CCC[C@@]65CCCCC5=O)nc43)cn2n1] | 1.15 | 703 | A |
| P-1f | 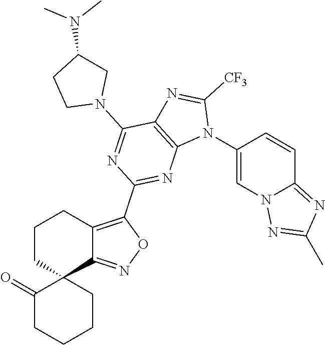 [SMILES: Cc1nc2ccc(-n3c(C(F)(F)F)nc4c(N5CC[C@H](N(C)C)C5)nc(-c5onc6c5CCC[C@@]65CCCCC5=O)nc43)cn2n1] | 1.04 | 661 | A |
| P-1g | 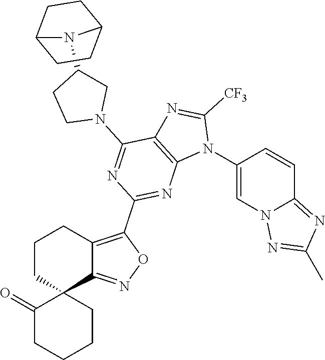 | 1.08 | 687 | A |
| P-1h | 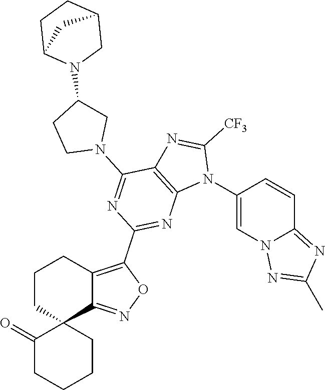 [SMILES: Cc1nc2ccc(-n3c(C(F)(F)F)nc4c(N5CC[C@H](N6C[C@@H]7CC[C@H]6C7)C5)nc(-c5onc6c5CCC[C@@]65CCCCC5=O)nc43)cn2n1] | 1.11 | 687 | A |
| P-1i | 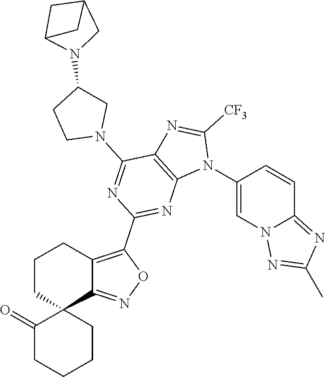 [SMILES: Cc1nc2ccc(-n3c(C(F)(F)F)nc4c(N5CC[C@H](N6CC7CC6C7)C5)nc(-c5onc6c5CCC[C@@]65CCCCC5=O)nc43)cn2n1] | 1.09 | 673 | A |
| P-1j | 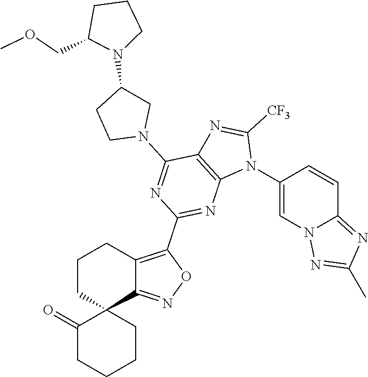 | 1.11 | 705 | A |
| P-1k | 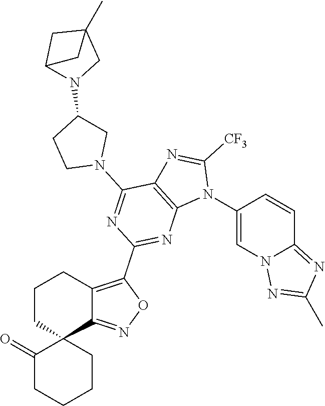 [SMILES: Cc1nc2ccc(-n3c(C(F)(F)F)nc4c(N5CC[C@H](N6CC7(C)CC6C7)C5)nc(-c5onc6c5CCC[C@@]65CCCCC5=O)nc43)cn2n1] | 1.05 | 687 | A |
| P-1l | 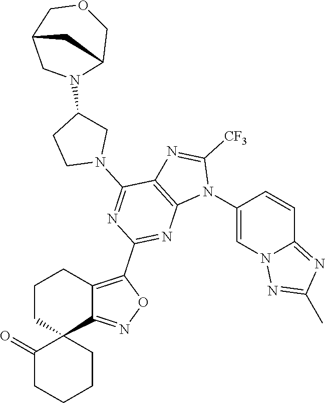 [SMILES: Cc1nc2ccc(-n3c(C(F)(F)F)nc4c(N5CC[C@H](N6C[C@@H]7COC[C@H]6C7)C5)nc(-c5onc6c5CCC[C@@]65CCCCC5=O)nc43)cn2n1] | 1.09 | 703 | A |
| P-1m | 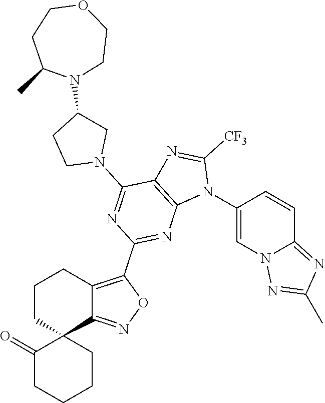 [SMILES: Cc1nc2ccc(-n3c(C(F)(F)F)nc4c(N5CC[C@H](N6CCOCC[C@@H]6C)C5)nc(-c5onc6c5CCC[C@@]65CCCCC5=O)nc43)cn2n1] | 1.09 | 705 | A |
| P-1n | 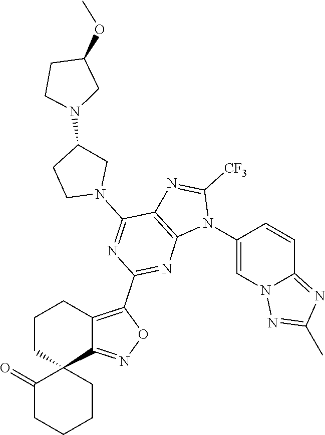 [SMILES: CO[C@@H]1CCN([C@H]2CCN(c3nc(-c4onc5c4CCC[C@@]54CCCCC4=O)nc4c3nc(C(F)(F)F)n4-c3ccc4nc(C)nn4c3)C2)C1] | 1.04 | 691 | 4 |
| P-1o | 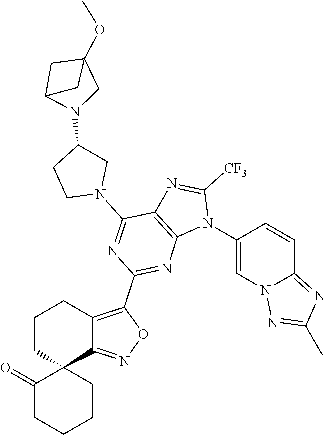 [SMILES: COC12CC(C1)N([C@H]1CCN(c3nc(-c4onc5c4CCC[C@@]54CCCCC4=O)nc4c3nc(C(F)(F)F)n4-c3ccc4nc(C)nn4c3)C1)C2] | 1.06 | 703 | 4 |
| P-1p | 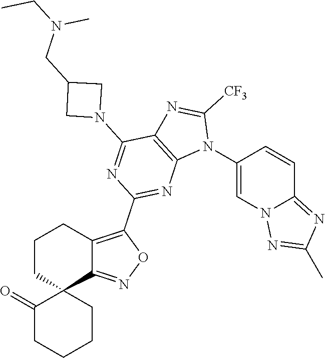 [SMILES: CCN(C)CC1CN(c2nc(-c3onc4c3CCC[C@@]43CCCCC3=O)nc3c2nc(C(F)(F)F)n3-c2ccc3nc(C)nn3c2)C1] | 0.81 | 667 | 4 |
| P-1q | 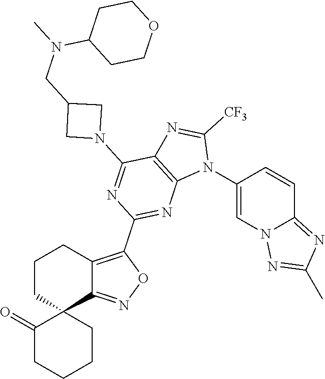 [SMILES: Cc1nc2ccc(-n3c(C(F)(F)F)nc4c(N5CC(CN(C)C6CCOCC6)C5)nc(-c5onc6c5CCC[C@@]65CCCCC5=O)nc43)cn2n1] | 0.95 | 705 | 4 |
| P-1r | 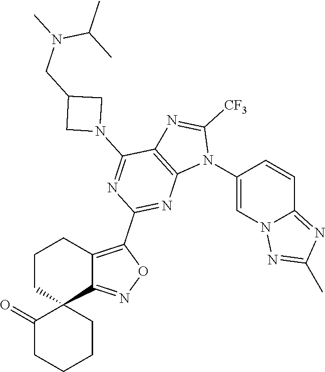 [SMILES: Cc1nc2ccc(-n3c(C(F)(F)F)nc4c(N5CC(CN(C)C(C)C)C5)nc(-c5onc6c5CCC[C@@]65CCCCC5=O)nc43)cn2n1] | 1.01 | 663 | A |
| P-1s | 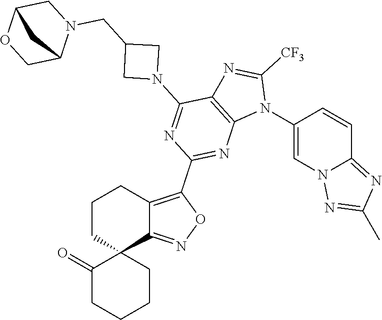 [SMILES: Cc1nc2ccc(-n3c(C(F)(F)F)nc4c(N5CC(CN6C[C@@H]7C[C@H]6CO7)C5)nc(-c5onc6c5CCC[C@@]65CCCCC5=O)nc43)cn2n1] | 0.912 | 689 | A |
| P-1t | 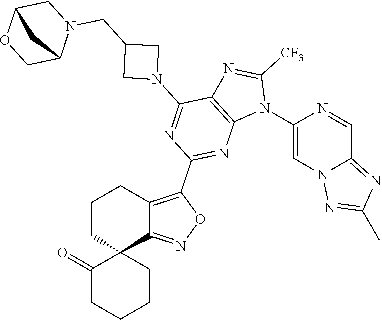 [SMILES: Cc1nc2cnc(-n3c(C(F)(F)F)nc4c(N5CC(CN6C[C@@H]7C[C@H]6CO7)C5)nc(-c5onc6c5CCC[C@@]65CCCCC5=O)nc43)cn2n1] | 0.94 | 690 | A |
| P-1u | 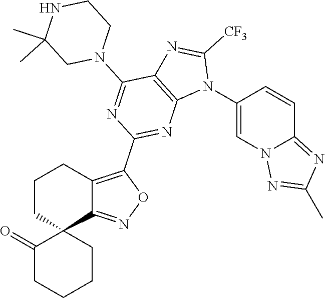 [SMILES: Cc1nc2ccc(-n3c(C(F)(F)F)nc4c(N5CCNC(C)(C)C5)nc(-c5onc6c5CCC[C@@]65CCCCC5=O)nc43)cn2n1] | 1.13 | 731 | A |
| P-1v | 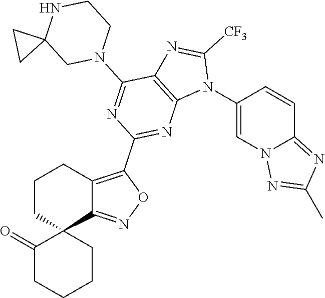 [SMILES: Cc1nc2ccc(-n3c(C(F)(F)F)nc4c(N5CCNC6(CC6)C5)nc(-c5onc6c5CCC[C@@]65CCCCC5=O)nc43)cn2n1] | 0.96 | 633 | A |
| P-1w | 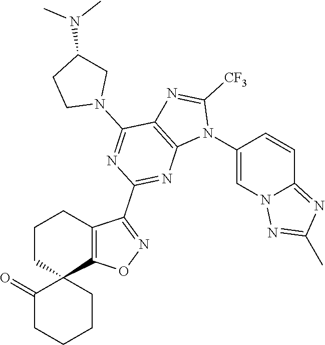 [SMILES: Cc1nc2ccc(-n3c(C(F)(F)F)nc4c(N5CC[C@H](N(C)C)C5)nc(-c5noc6c5CCC[C@@]65CCCCC5=O)nc43)cn2n1] | 1.00 | 635 | A |
| P-1x | 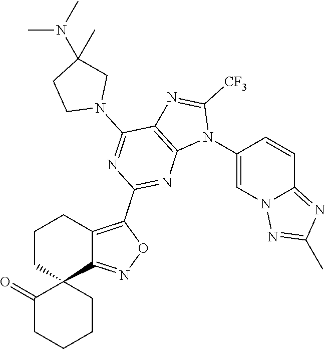 [SMILES: Cc1nc2ccc(-n3c(C(F)(F)F)nc4c(N5CCC(C)(N(C)C)C5)nc(-c5onc6c5CCC[C@@]65CCCCC5=O)nc43)cn2n1] | 1.03 | 648 | A |
| P-1y | 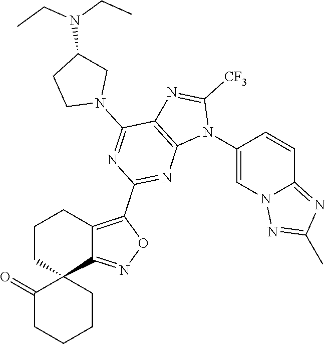 [SMILES: CCN(CC)[C@H]1CCN(c2nc(-c3onc4c3CCC[C@@]43CCCCC3=O)nc3c2nc(C(F)(F)F)n3-c2ccc3nc(C)nn3c2)C1] | 1.12 | 663 | A |
| P-1z | 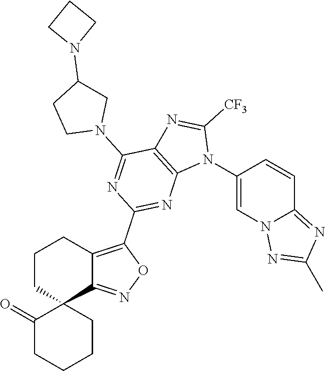 [SMILES: Cc1nc2ccc(-n3c(C(F)(F)F)nc4c(N5CCC(N6CCC6)C5)nc(-c5onc6c5CCC[C@@]65CCCCC5=O)nc43)cn2n1] | 1.01 | 647 | A |
| P-1aa | 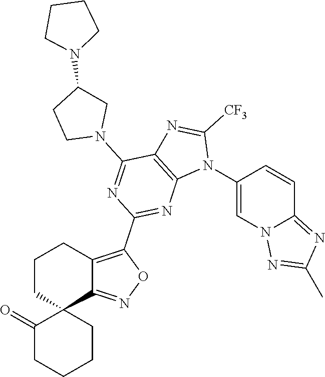 [SMILES: Cc1nc2ccc(-n3c(C(F)(F)F)nc4c(N5CC[C@H](N6CCCC6)C5)nc(-c5onc6c5CCC[C@@]65CCCCC5=O)nc43)cn2n1] | 1.03 | 635 | A |
| P-1ab | 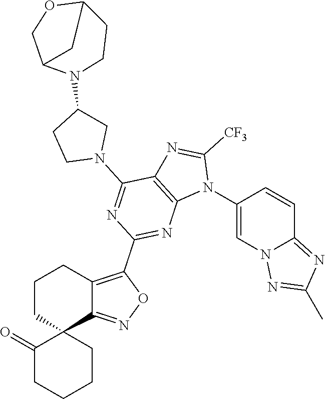 [SMILES: Cc1nc2ccc(-n3c(C(F)(F)F)nc4c(N5CC[C@H](N6CCC7CC6CO7)C5)nc(-c5onc6c5CCC[C@@]65CCCCC5=O)nc43)cn2n1] | 1.09 | 703 | A |
| P-1ac | 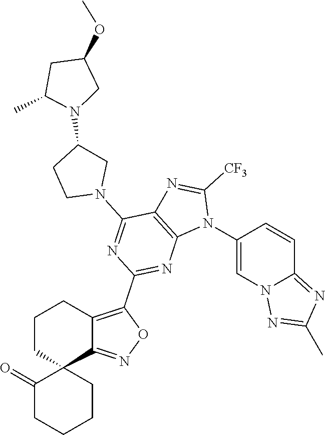 [SMILES: CO[C@@H]1C[C@@H](C)N([C@H]2CCN(c3nc(-c4onc5c4CCC[C@@]54CCCCC4=O)nc4c3nc(C(F)(F)F)n4-c3ccc4nc(C)nn4c3)C2)C1] | 1.11 | 705 | A |
| P-1ad | 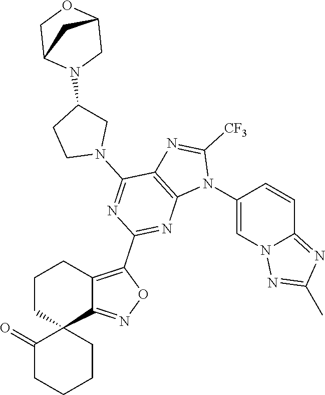 [SMILES: Cc1nc2ccc(-n3c(C(F)(F)F)nc4c(N5CC[C@H](N6C[C@@H]7C[C@H]6CO7)C5)nc(-c5onc6c5CCC[C@@]65CCCCC5=O)nc43)cn2n1] | 1.11 | 689 | A |
| P-1ae | 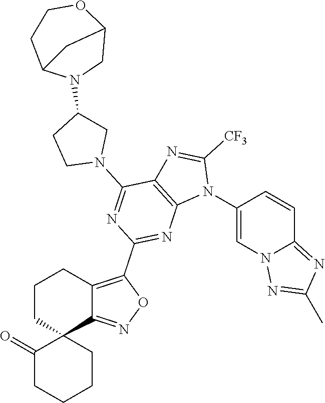 [SMILES: Cc1nc2ccc(-n3c(C(F)(F)F)nc4c(N5CC[C@H](N6CC7CC6CCO7)C5)nc(-c5onc6c5CCC[C@@]65CCCCC5=O)nc43)cn2n1] | 1.14 | 703 | A |
| P-1af | 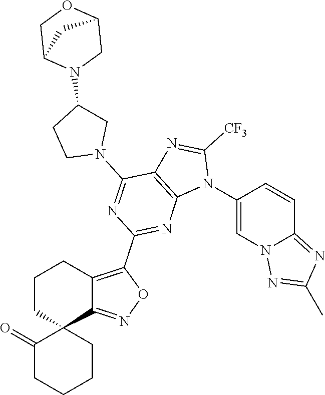 [SMILES: Cc1nc2ccc(-n3c(C(F)(F)F)nc4c(N5CC[C@H](N6C[C@H]7C[C@@H]6CO7)C5)nc(-c5onc6c5CCC[C@@]65CCCCC5=O)nc43)cn2n1] | 1.12 | 689 | A |
| P-1ag | 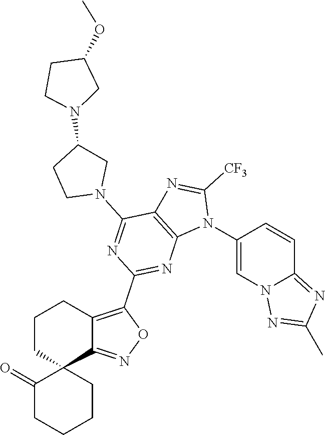 [SMILES: CO[C@H]1CCN([C@H]2CCN(c3nc(-c4onc5c4CCC[C@@]54CCCCC4=O)nc4c3nc(C(F)(F)F)n4-c3ccc4nc(C)nn4c3)C2)C1] | 1.07 | 691 | A |
| P-1ah | 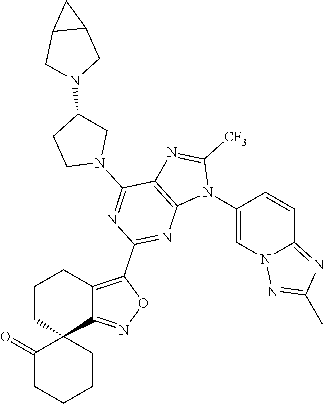 [SMILES: Cc1nc2ccc(-n3c(C(F)(F)F)nc4c(N5CC[C@H](N6CC7CC7C6)C5)nc(-c5onc6c5CCC[C@@]65CCCCC5=O)nc43)cn2n1] | 1.13 | 673 | A |
| P-1ai | 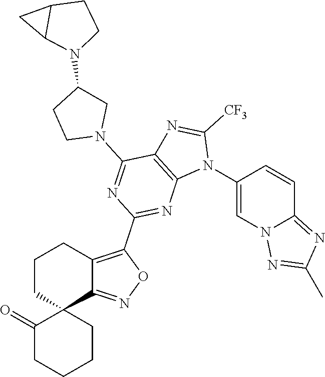 [SMILES: Cc1nc2ccc(-n3c(C(F)(F)F)nc4c(N5CC[C@H](N6CCC7CC76)C5)nc(-c5onc6c5CCC[C@@]65CCCCC5=O)nc43)cn2n1] | 1.14 | 673 | A |
| P-1aj | 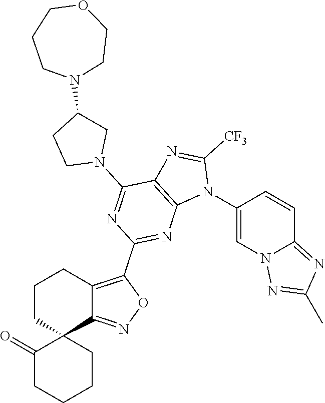 [SMILES: Cc1nc2ccc(-n3c(C(F)(F)F)nc4c(N5CC[C@H](N6CCCOCC6)C5)nc(-c5onc6c5CCC[C@@]65CCCCC5=O)nc43)cn2n1] | 1.11 | 691 | A |
| P-1ak | 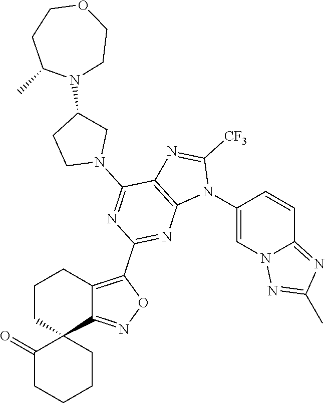 [SMILES: Cc1nc2ccc(-n3c(C(F)(F)F)nc4c(N5CC[C@H](N6CCOCC[C@H]6C)C5)nc(-c5onc6c5CCC[C@@]65CCCCC5=O)nc43)cn2n1] | 1.12 | 705 | A |
| P-1al | 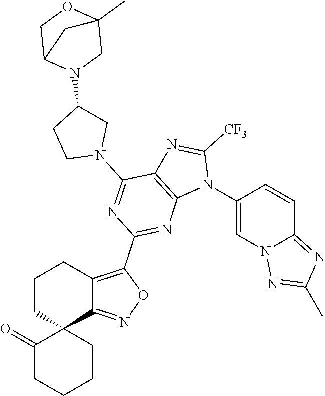 [SMILES: Cc1nc2ccc(-n3c(C(F)(F)F)nc4c(N5CC[C@H](N6CC7(C)CC6CO7)C5)nc(-c5onc6c5CCC[C@@]65CCCCC5=O)nc43)cn2n1] | 1.12 | 703 | A |
| P-1am | 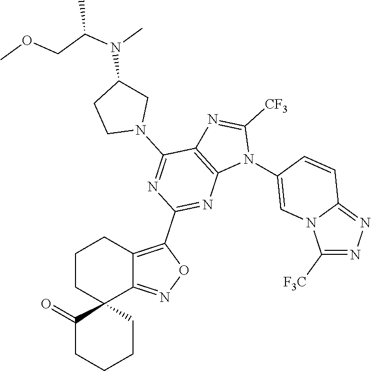 [SMILES: COC[C@H](C)N(C)[C@H]1CCN(c2nc(-c3onc4c3CCC[C@@]43CCCCC3=O)nc3c2nc(C(F)(F)F)n3-c2ccc3nnc(C(F)(F)F)n3c2)C1] | 1.13 | 747 | A |
| P-1an | 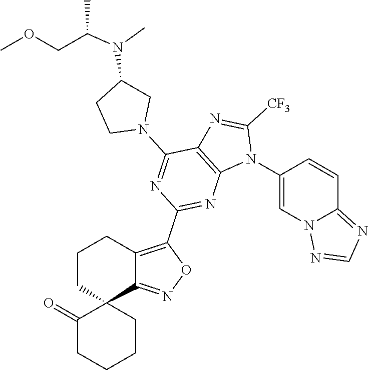 [SMILES: COC[C@H](C)N(C)[C@H]1CCN(c2nc(-c3onc4c3CCC[C@@]43CCCCC3=O)nc3c2nc(C(F)(F)F)n3-c2ccc3ncnn3c2)C1] | 1.11 | 679 | A |
| P-1ao | 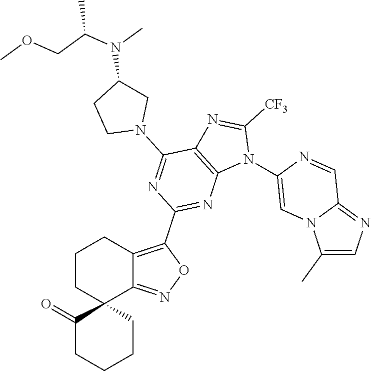 [SMILES: COC[C@H](C)N(C)[C@H]1CCN(c2nc(-c3onc4c3CCC[C@@]43CCCCC3=O)nc3c2nc(C(F)(F)F)n3-c2cn3c(C)cnc3cn2)C1] | 1.15 | 693 | A |
| P-1ap | 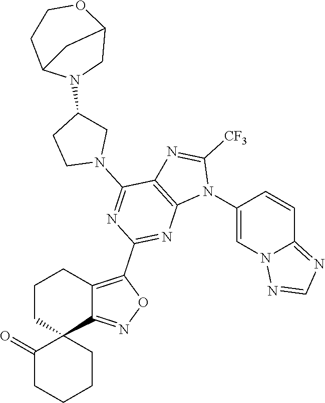 [SMILES: O=C1CCCC[C@@]12CCCc1c2noc1-c1nc(N2CC[C@H](N3CC4CC3CCO4)C2)c2nc(C(F)(F)F)n(-c3ccc4ncnn4c3)c2n1] | 1.11 | 689 | A |
| P-1aq | 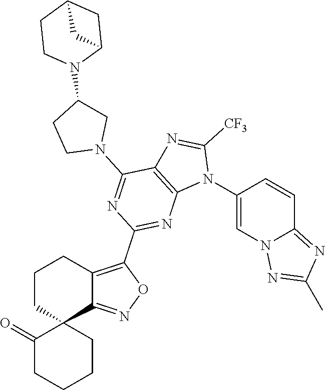 [SMILES: Cc1nc2ccc(-n3c(C(F)(F)F)nc4c(N5CC[C@H](N6CC[C@H]7C[C@@H]6C7)C5)nc(-c5onc6c5CCC[C@@]65CCCCC5=O)nc43)cn2n1] | 1.09 | 687 | A |
| P-1ar | 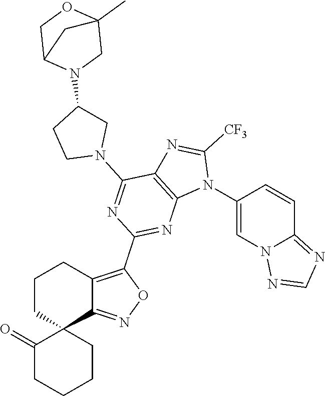 [SMILES: CC12CC(CO1)N([C@H]1CCN(c3nc(-c4onc5c4CCC[C@@]54CCCCC4=O)nc4c3nc(C(F)(F)F)n4-c3ccc4ncnn4c3)C1)C2] | 1.14 | 689 | A |
| P-1as | 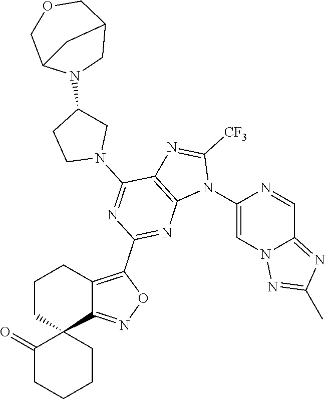 [SMILES: Cc1nc2cnc(-n3c(C(F)(F)F)nc4c(N5CC[C@H](N6CC7COCC6C7)C5)nc(-c5onc6c5CCC[C@@]65CCCCC5=O)nc43)cn2n1] | 1.13 | 704 | A |
| P-1at | 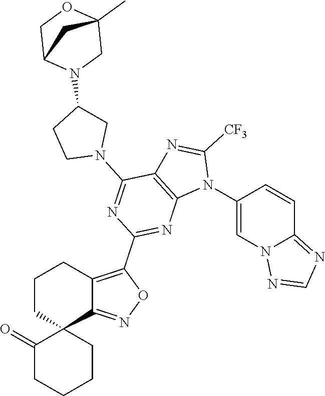 [SMILES: C[C@@]12C[C@@H](CO1)N([C@H]1CCN(c3nc(-c4onc5c4CCC[C@@]54CCCCC4=O)nc4c3nc(C(F)(F)F)n4-c3ccc4ncnn4c3)C1)C2] | 1.09 | 689 | A |
| P-1au | 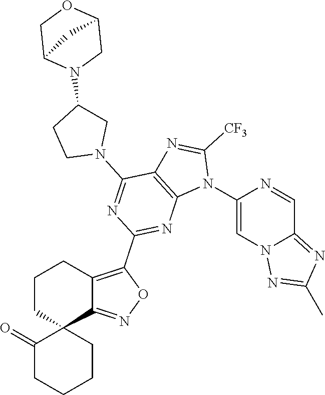 [SMILES: Cc1nc2cnc(-n3c(C(F)(F)F)nc4c(N5CC[C@H](N6C[C@H]7C[C@@H]6CO7)C5)nc(-c5onc6c5CCC[C@@]65CCCCC5=O)nc43)cn2n1] | 1.10 | 703 | A |
| P-1av | 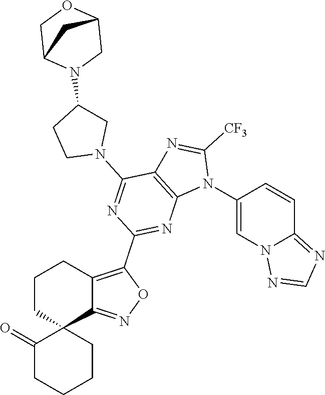 [SMILES: O=C1CCCC[C@@]12CCCc1c2noc1-c1nc(N2CC[C@H](N3C[C@@H]4C[C@H]3CO4)C2)c2nc(C(F)(F)F)n(-c3ccc4ncnn4c3)c2n1] | 1.08 | 675 | A |
| P-1aw | 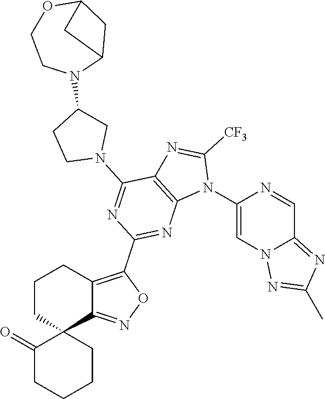 [SMILES: Cc1nc2cnc(-n3c(C(F)(F)F)nc4c(N5CC[C@H](N6CCOC7CC6C7)C5)nc(-c5onc6c5CCC[C@@]65CCCCC5=O)nc43)cn2n1] | 0.98 | 704 | A |
| P-1ay | 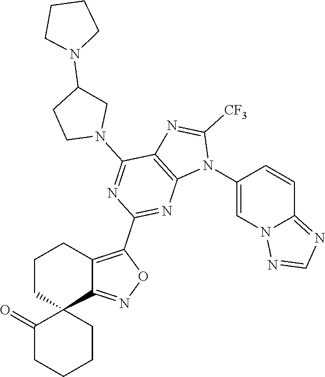 [SMILES: O=C1CCCC[C@@]12CCCc1c2noc1-c1nc(N2CCC(N3CCCC3)C2)c2nc(C(F)(F)F)n(-c3ccc4ncnn4c3)c2n1] | 1.17 | 647 | A |
| P-1az | 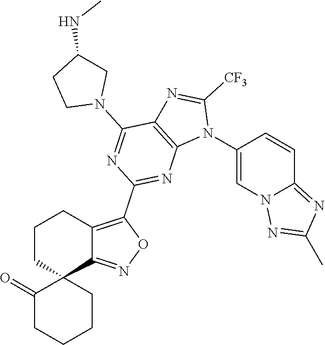 [SMILES: CN[C@H]1CCN(c2nc(-c3onc4c3CCC[C@@]43CCCCC3=O)nc3c2nc(C(F)(F)F)n3-c2ccc3nc(C)nn3c2)C1] | 1.35 | 621 | A |
| P-1ba | 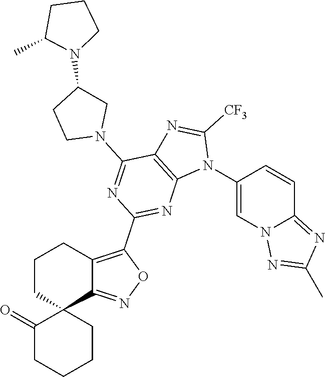 [SMILES: Cc1nc2ccc(-n3c(C(F)(F)F)nc4c(N5CC[C@H](N6CCC[C@H]6C)C5)nc(-c5onc6c5CCC[C@@]65CCCCC5=O)nc43)cn2n1] | 1.05 | 675 | A |
| P-1bb | 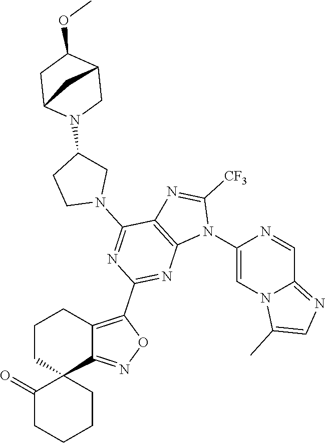 [SMILES: CO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3c(C)cnc3cn2)C1] | 1.03 | 717 | C |
| P-1bc | 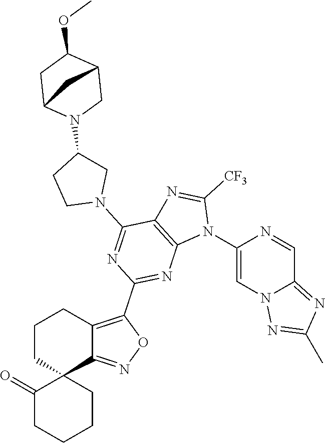 [SMILES: CO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3nc(C)nc3cn2)C1] | 1.11 | 718 | C |
| P-1bd | 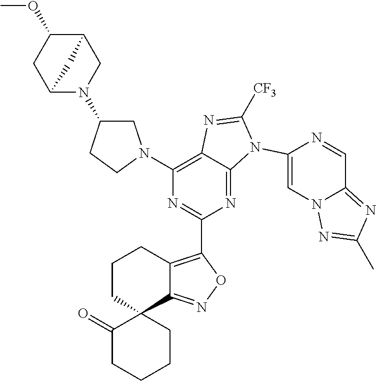 | 1.11 | 718 | C |
| P-1be | 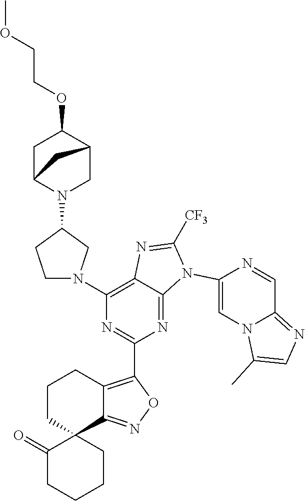 [SMILES: COCCO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3c(C)cnc3cn2)C1] | 1.02 | 761 | A |

### Coupling of Amines A-1 and Anilines C-1 to Building Block F-1

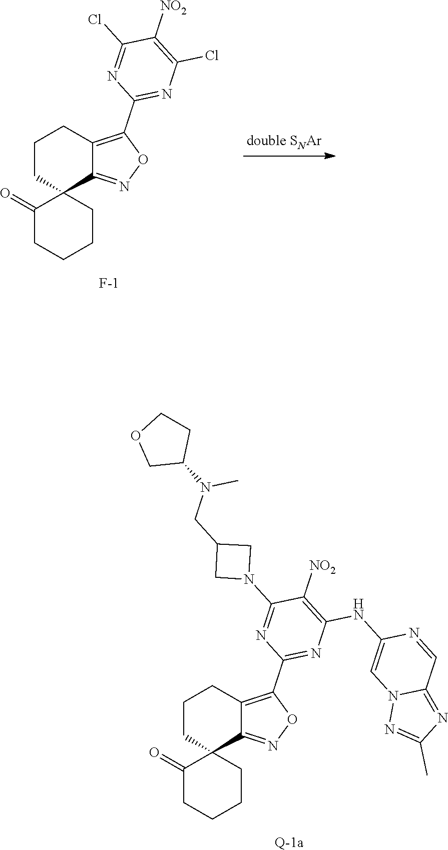

F-1a (250 mg, 0.59 mmol, 1 equiv) is dissolved in CH₂Cl₂ (1 mL) and the solution was cooled to −10° C. A solution of the amine coupling partner (3S)—N-(azetidin-3-ylmethyl)-N-methyl-tetrahydrofuran-3-amine; dihydrochloride (152 mg, 0.59 mmol, 1 equiv) in CH₂Cl₂ (1 mL) and DIPEA (0.35 mL, 2.09 mmol, 3.5 equiv) is added dropwise to this solution. After stirring for 10 min at −10° C., the mixture is evaporated to dryness and the crude was redissolved in THF (5 mL) and cooled to −10° C. Aniline coupling partner 2-methyl-[1,2,4]triazolo[1,5-A]pyrazin-6-amine (113 mg, 0.71 mmol, 1.2 equiv), followed by LiHMDS solution (1 M, 2.92 mL, 2.92 mmol, 4.9 equiv) were added and the solution was stirred for 30 min. The reaction mixture was diluted with CH₂Cl₂ and aqeuous NaHCO₃ solution and extracted with CH₂Cl₂ three times. The combined extracts are dried with Na₂SO₄ and evaporated to dryness. The crude product is purified by RP-chromatography to give the desired product product Q-1a (HPLC method A, tᵣₑₜ=0.98 min; [M+H]=644).

The following intermediate compounds Q-1 (Table 17) are obtained analogously using the corresponding amine and aniline as coupling partners.

TABLE 17

| | | | | HPLC |
|---|---|---|---|---|
| # | structure | tᵣₑₜ [min] | [M + H]⁺ | method |
| Q-1b | 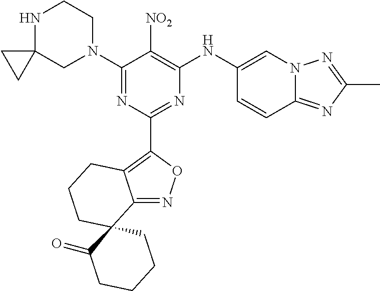 [SMILES: Cc1nc2ccc(Nc3nc(-c4onc5c4CCC[C@@]54CCCCC4=O)nc(N4CCNC5(CC5)C4)c3[N+](=O)[O-])cn2n1] | 1.00 | 585 | A |
| Q-1c | 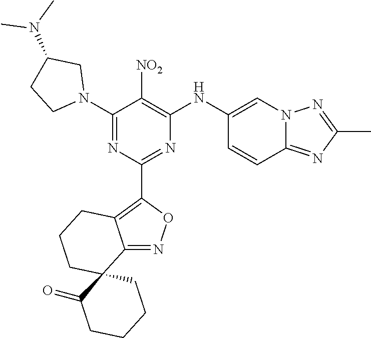 | 0.99 | 587 | A |
| Q-1d | 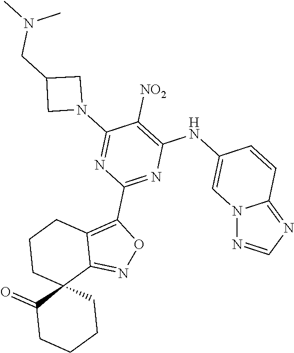 [SMILES: CN(C)CC1CN(c2nc(-c3onc4c3CCC[C@@]43CCCCC3=O)nc(Nc3ccc4ncnn4c3)c2[N+](=O)[O-])C1] | 0.90 | 573 | A |
| Q-1e | 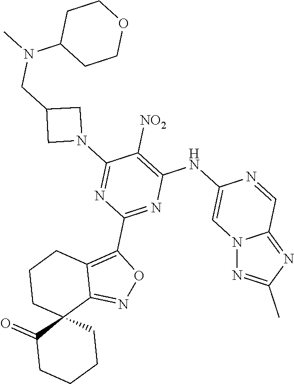 [SMILES: Cc1nc2cnc(Nc3nc(-c4onc5c4CCC[C@@]54CCCCC4=O)nc(N4CC(CN(C)C5CCOCC5)C4)c3[N+](=O)[O-])cn2n1] | 1.04 | 658 | A |
| Q-1f | 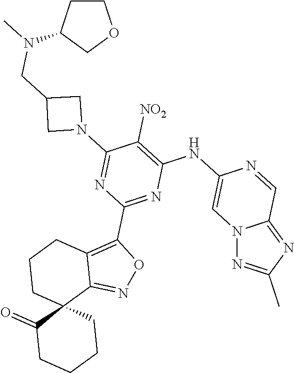 [SMILES: Cc1nc2cnc(Nc3nc(-c4onc5c4CCC[C@@]54CCCCC4=O)nc(N4CC(CN(C)[C@@H]5CCOC5)C4)c3[N+](=O)[O-])cn2n1] | 0.98 | 644 | A |
| Q-1g | 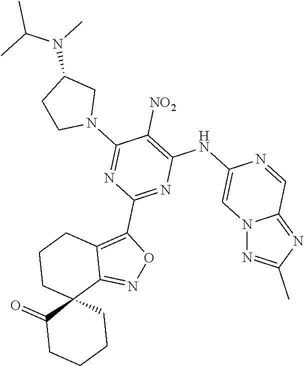 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cn2n1] | 1.06 | 616 | A |
| Q-1h | 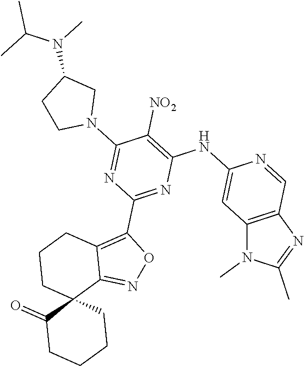 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cc2n1C] | 1.04 | 629 | A |
| Q-1i | 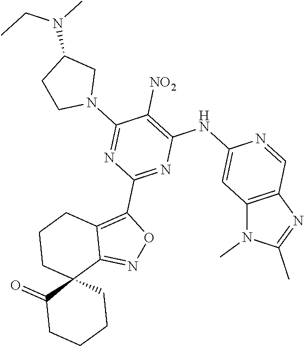 [SMILES: CCN(C)[C@H]1CCN(c2nc(-c3onc4c3CCC[C@@]43CCCCC3=O)nc(Nc3cc4c(cn3)nc(C)n4C)c2[N+](=O)[O-])C1] | 1.02 | 615 | A |
| Q-1j | 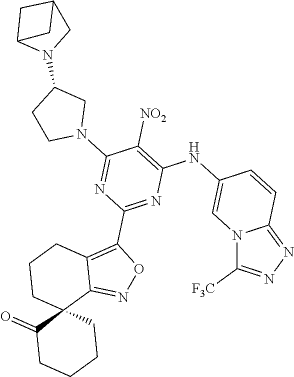 [SMILES: O=C1CCCC[C@@]12CCCc1c2noc1-c1nc(Nc2ccc3nnc(C(F)(F)F)n3c2)c([N+](=O)[O-])c(N2CC[C@H](N3CC4CC3C4)C2)n1] | 1.06 | 679 | A |
| Q-1k | 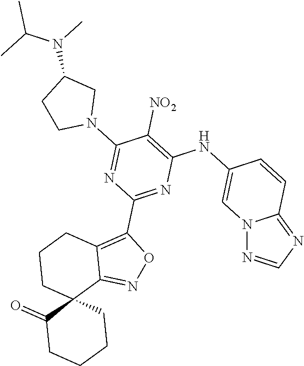 [SMILES: CC(C)N(C)[C@H]1CCN(c2nc(-c3onc4c3CCC[C@@]43CCCCC3=O)nc(Nc3ccc4ncnn4c3)c2[N+](=O)[O-])C1] | 1.16 | 601 | A |
| Q-1l | 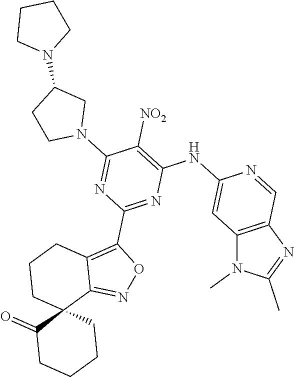 [SMILES: Cc1nc2cnc(Nc3nc(-c4onc5c4CCC[C@@]54CCCCC4=O)nc(N4CC[C@H](N5CCCC5)C4)c3[N+](=O)[O-])cc2n1C] | 1.06 | 627 | A |
| Q-1m | 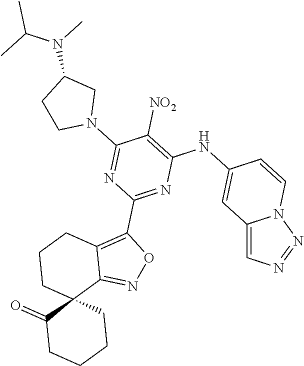 [SMILES: CC(C)N(C)[C@H]1CCN(c2nc(-c3onc4c3CCC[C@@]43CCCCC3=O)nc(Nc3ccn4nncc4c3)c2[N+](=O)[O-])C1] | 1.06 | 601 | A |
| Q-1n | 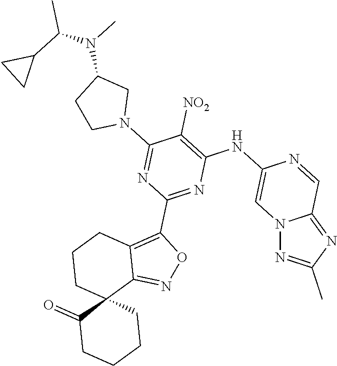 [SMILES: Cc1nc2cnc(Nc3nc(-c4onc5c4CCC[C@@]54CCCCC4=O)nc(N4CC[C@H](N(C)[C@@H](C)C5CC5)C4)c3[N+](=O)[O-])cn2n1] | 1.18 | 642 | A |
| Q-1o | 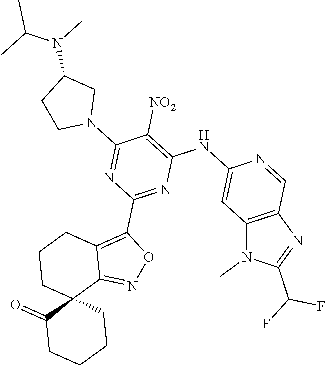 [SMILES: CC(C)N(C)[C@H]1CCN(c2nc(-c3onc4c3CCC[C@@]43CCCCC3=O)nc(Nc3cc4c(cn3)nc(C(F)F)n4C)c2[N+](=O)[O-])C1] | 1.13 | 665 | A |
| Q-1p | 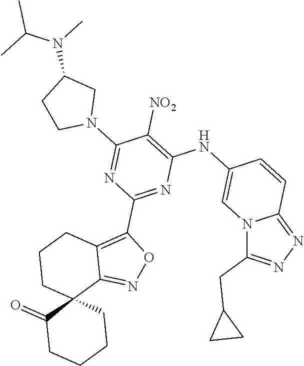 [SMILES: CC(C)N(C)[C@H]1CCN(c2nc(-c3onc4c3CCC[C@@]43CCCCC3=O)nc(Nc3ccc4nnc(CC5CC5)n4c3)c2[N+](=O)[O-])C1] | 1.11 | 655 | A |
| Q-1q | 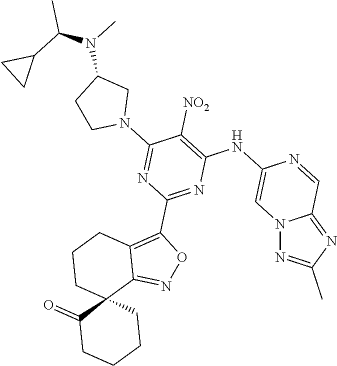 [SMILES: Cc1nc2cnc(Nc3nc(-c4onc5c4CCC[C@@]54CCCCC4=O)nc(N4CC[C@H](N(C)[C@H](C)C5CC5)C4)c3[N+](=O)[O-])cn2n1] | 1.17 | 642 | A |
| Q-1r | 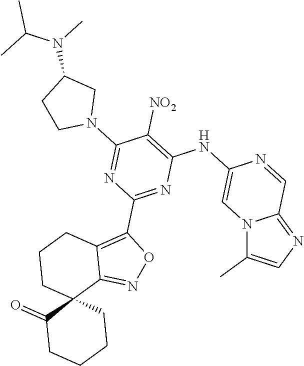 [SMILES: Cc1cnc2cnc(Nc3nc(-c4onc5c4CCC[C@@]54CCCCC4=O)nc(N4CC[C@H](N(C)C(C)C)C4)c3[N+](=O)[O-])cn12] | 1.10 | 615 | A |
| Q-1s | 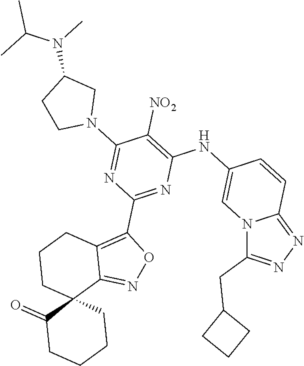 [SMILES: CC(C)N(C)[C@H]1CCN(c2nc(-c3onc4c3CCC[C@@]43CCCCC3=O)nc(Nc3ccc4nnc(CC5CCC5)n4c3)c2[N+](=O)[O-])C1] | 1.14 | 669 | A |
| Q-1t | 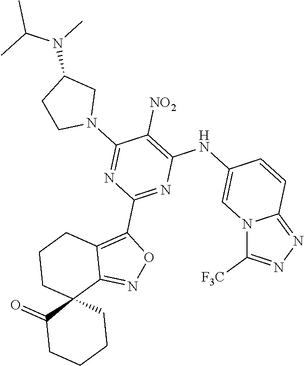 [SMILES: CC(C)N(C)[C@H]1CCN(c2nc(-c3onc4c3CCC[C@@]43CCCCC3=O)nc(Nc3ccc4nnc(C(F)(F)F)n4c3)c2[N+](=O)[O-])C1] | 1.10 | 669 | A |
| Q-1u | 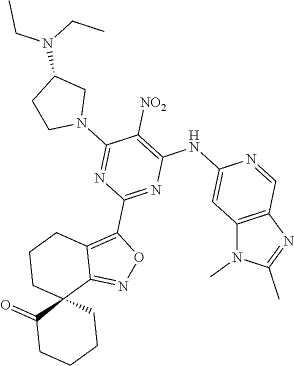 [SMILES: CCN(CC)[C@H]1CCN(c2nc(-c3onc4c3CCC[C@@]43CCCCC3=O)nc(Nc3cc4c(cn3)nc(C)n4C)c2[N+](=O)[O-])C1] | 1.07 | 629 | A |
| Q-1v | 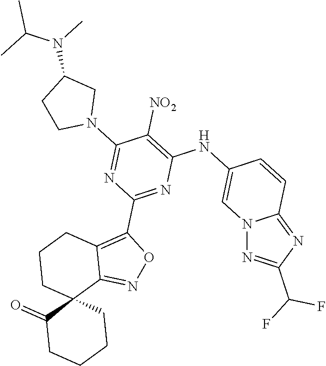 [SMILES: CC(C)N(C)[C@H]1CCN(c2nc(-c3onc4c3CCC[C@@]43CCCCC3=O)nc(Nc3ccc4nc(C(F)F)nn4c3)c2[N+](=O)[O-])C1] | 1.07 | 651 | A |
| Q-1w | 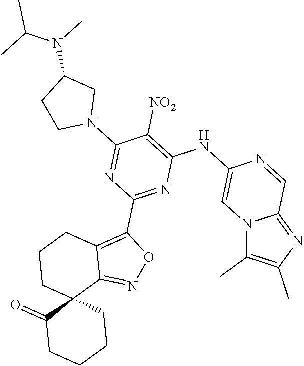 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cn2c1C] | 1.14 | 629 | A |
| Q-1x | 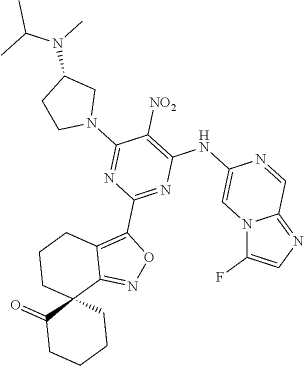 [SMILES: CC(C)N(C)[C@H]1CCN(c2nc(-c3onc4c3CCC[C@@]43CCCCC3=O)nc(Nc3cn4c(F)cnc4cn3)c2[N+](=O)[O-])C1] | 1.11 | 619 | A |
| Q-1y | 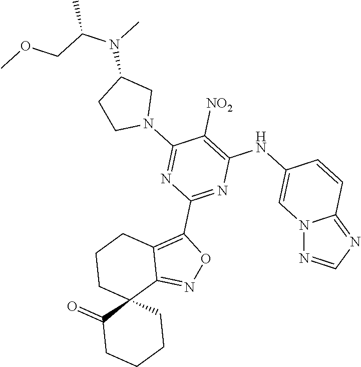 [SMILES: COC[C@H](C)N(C)[C@H]1CCN(c2nc(-c3onc4c3CCC[C@@]43CCCCC3=O)nc(Nc3ccc4ncnn4c3)c2[N+](=O)[O-])C1] | 1.11 | 679 | A |
| Q-1z | 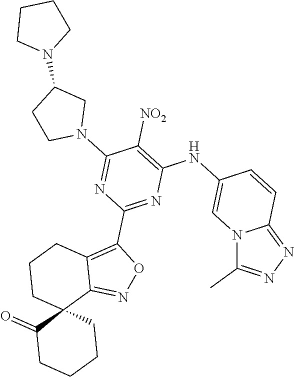 [SMILES: Cc1nnc2ccc(Nc3nc(-c4onc5c4CCC[C@@]54CCCCC4=O)nc(N4CC[C@H](N5CCCC5)C4)c3[N+](=O)[O-])cn12] | 0.99 | 613 | A |
| Q-1aa | 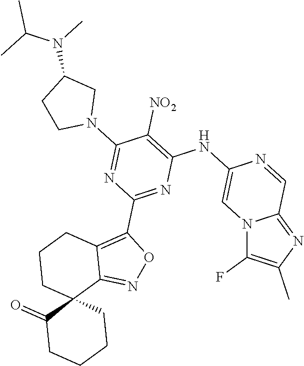 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cn2c1F] | 1.01 | 633 | A |
| Q-1ab | 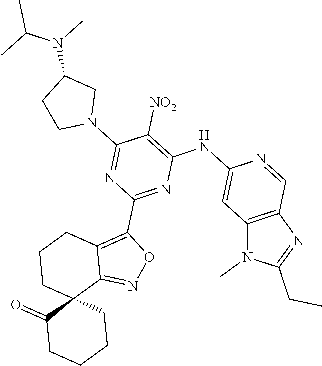 [SMILES: CCc1nc2cnc(Nc3nc(-c4onc5c4CCC[C@@]54CCCCC4=O)nc(N4CC[C@H](N(C)C(C)C)C4)c3[N+](=O)[O-])cc2n1C] | 1.14 | 633 | A |
| Q-1ac | 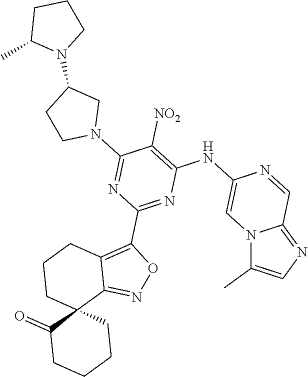 [SMILES: Cc1cnc2cnc(Nc3nc(-c4onc5c4CCC[C@@]54CCCCC4=O)nc(N4CC[C@H](N5CCC[C@H]5C)C4)c3[N+](=O)[O-])cn12] | 1.11 | 627 | A |
| Q-1ad | 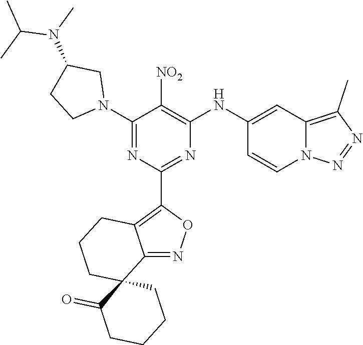 [SMILES: Cc1nnn2ccc(Nc3nc(-c4onc5c4CCC[C@@]54CCCCC4=O)nc(N4CC[C@H](N(C)C(C)C)C4)c3[N+](=O)[O-])cc12] | 1.07 | 615 | A |
| Q-1ae | 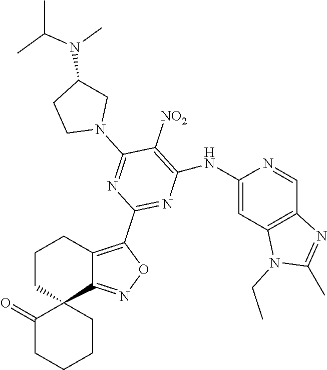 [SMILES: CCn1c(C)nc2cnc(Nc3nc(-c4onc5c4CCC[C@@]54CCCCC4=O)nc(N4CC[C@H](N(C)C(C)C)C4)c3[N+](=O)[O-])cc21] | 1.1 | 643 | A |
| Q-1af | 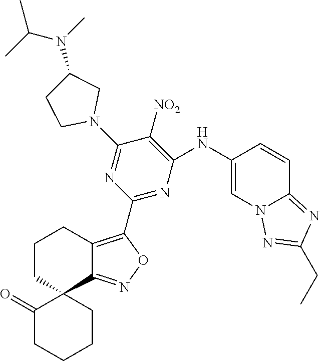 [SMILES: CCc1nc2ccc(Nc3nc(-c4onc5c4CCC[C@@]54CCCCC4=O)nc(N4CC[C@H](N(C)C(C)C)C4)c3[N+](=O)[O-])cn2n1] | 1.10 | 629 | A |
| Q-1ag | 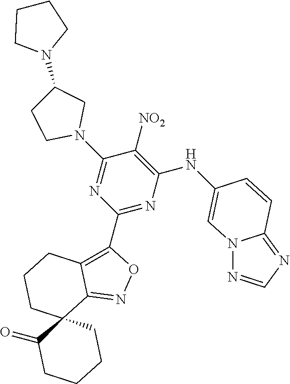 [SMILES: O=C1CCCC[C@@]12CCCc1c2noc1-c1nc(Nc2ccc3ncnn3c2)c([N+](=O)[O-])c(N2CC[C@H](N3CCCC3)C2)n1] | 1.05 | 599 | A |
| Q-1ah | 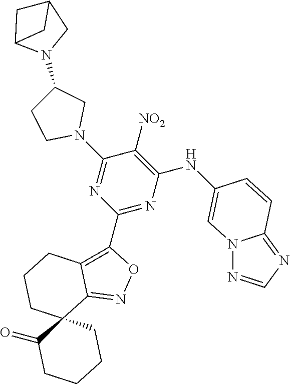 [SMILES: O=C1CCCC[C@@]12CCCc1c2noc1-c1nc(Nc2ccc3ncnn3c2)c([N+](=O)[O-])c(N2CC[C@H](N3CC4CC3C4)C2)n1] | 1.11 | 611 | A |
| Q-1ai | 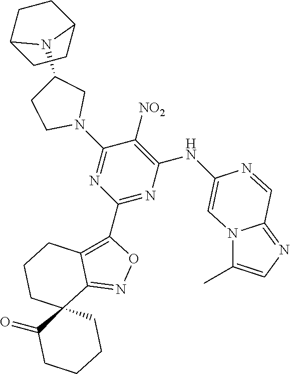 [SMILES: Cc1cnc2cnc(Nc3nc(-c4onc5c4CCC[C@@]54CCCCC4=O)nc(N4CC[C@H](N5C6CCC5CC6)C4)c3[N+](=O)[O-])cn12] | 1.04 | 639 | A |
| Q-1aj | 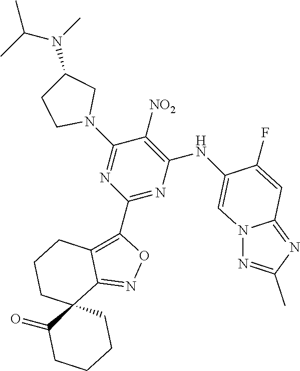 [SMILES: Cc1nc2cc(F)c(Nc3nc(-c4onc5c4CCC[C@@]54CCCCC4=O)nc(N4CC[C@H](N(C)C(C)C)C4)c3[N+](=O)[O-])cn2n1] | 1.09 | 633 | A |
| Q-1ak | 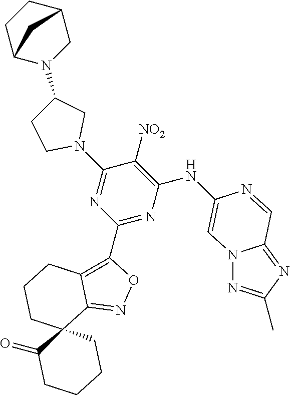 [SMILES: Cc1nc2cnc(Nc3nc(-c4onc5c4CCC[C@@]54CCCCC4=O)nc(N4CC[C@H](N5C[C@H]6CC[C@@H]5C6)C4)c3[N+](=O)[O-])cn2n1] | 1.10 | 640 | A |
| Q-1al | 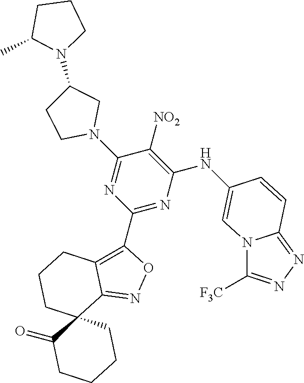 [SMILES: C[C@@H]1CCCN1[C@H]1CCN(c2nc(-c3onc4c3CCC[C@@]43CCCCC3=O)nc(Nc3ccc4nnc(C(F)(F)F)n4c3)c2[N+](=O)[O-])C1] | 1.12 | 681 | A |
| Q-1am | 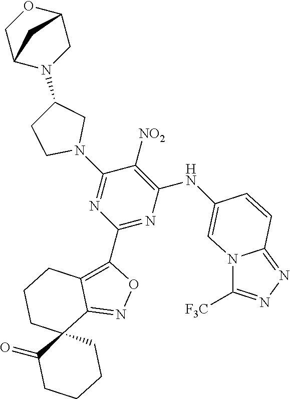 [SMILES: O=C1CCCC[C@@]12CCCc1c2noc1-c1nc(Nc2ccc3nnc(C(F)(F)F)n3c2)c([N+](=O)[O-])c(N2CC[C@H](N3C[C@@H]4C[C@H]3CO4)C2)n1] | 1.15 | 695 | A |
| Q-1an | 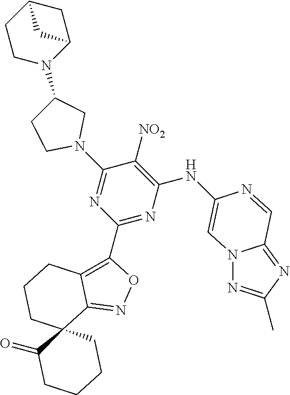 [SMILES: Cc1nc2cnc(Nc3nc(-c4onc5c4CCC[C@@]54CCCCC4=O)nc(N4CC[C@H](N5CC[C@H]6C[C@@H]5C6)C4)c3[N+](=O)[O-])cn2n1] | 1.19 | 640 | A |
| Q-1ao | 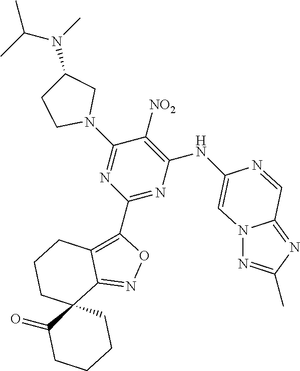 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cn2n1] | 1.12 | 616 | A |
| Q-1ap | 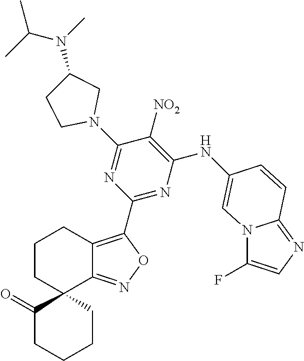 [SMILES: CC(C)N(C)[C@H]1CCN(c2nc(-c3onc4c3CCC[C@@]43CCCCC3=O)nc(Nc3ccc4ncc(F)n4c3)c2[N+](=O)[O-])C1] | 1.12 | 618 | A |
| Q-1aq | 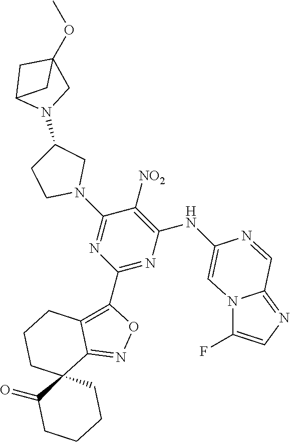 [SMILES: COC12CC(C1)N([C@H]1CCN(c3nc(-c4onc5c4CCC[C@@]54CCCCC4=O)nc(Nc4cn5c(F)cnc5cn4)c3[N+](=O)[O-])C1)C2] | 1.08 | 659 | A |
| Q-1ar | 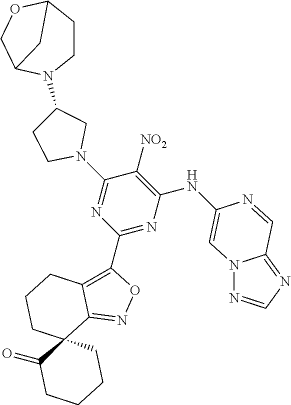 [SMILES: O=C1CCCC[C@@]12CCCc1c2noc1-c1nc(Nc2cn3ncnc3cn2)c([N+](=O)[O-])c(N2CC[C@H](N3CCC4CC3CO4)C2)n1] | 1.08 | 642 | A |
| Q-1as | 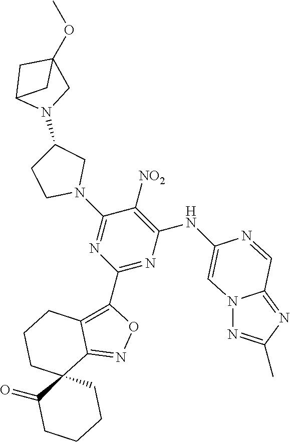 [SMILES: COC12CC(C1)N([C@H]1CCN(c3nc(-c4onc5c4CCC[C@@]54CCCCC4=O)nc(Nc4cn5nc(C)nc5cn4)c3[N+](=O)[O-])C1)C2] | 1.14 | 656 | A |
| Q-1at | 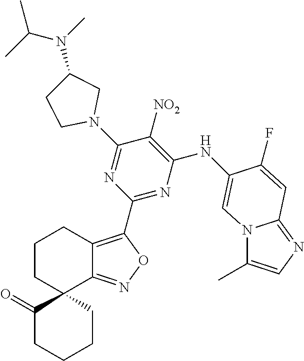 [SMILES: Cc1cnc2cc(F)c(Nc3nc(-c4onc5c4CCC[C@@]54CCCCC4=O)nc(N4CC[C@H](N(C)C(C)C)C4)c3[N+](=O)[O-])cn12] | 1.08 | 632 | A |
| Q-1au | 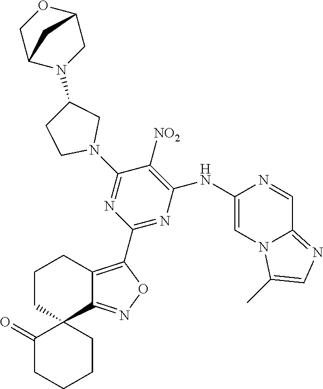 [SMILES: Cc1cnc2cnc(Nc3nc(-c4onc5c4CCC[C@@]54CCCCC4=O)nc(N4CC[C@H](N5C[C@@H]6C[C@H]5CO6)C4)c3[N+](=O)[O-])cn12] | 1.13 | 641 | A |
| Q-1av | 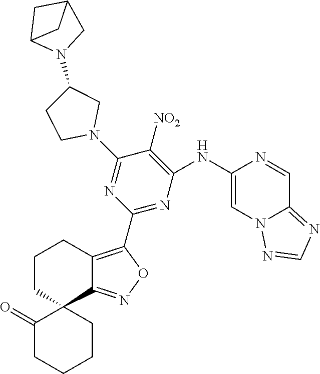 [SMILES: O=C1CCCC[C@@]12CCCc1c2noc1-c1nc(Nc2cn3ncnc3cn2)c([N+](=O)[O-])c(N2CC[C@H](N3CC4CC3C4)C2)n1] | 1.10 | 612 | A |
| Q-1aw | 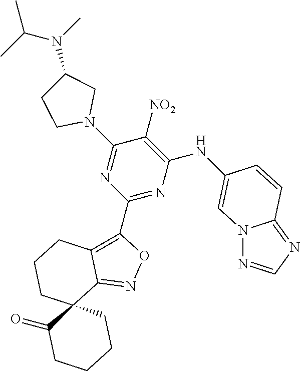 [SMILES: CC(C)N(C)[C@H]1CCN(c2nc(-c3onc4c3CCC[C@@]43CCCCC3=O)nc(Nc3ccc4ncnn4c3)c2[N+](=O)[O-])C1] | 1.04 | 601 | A |
| Q-1ax | 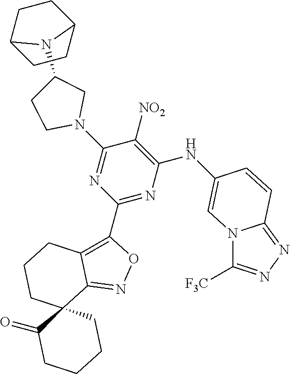 [SMILES: O=C1CCCC[C@@]12CCCc1c2noc1-c1nc(Nc2ccc3nnc(C(F)(F)F)n3c2)c([N+](=O)[O-])c(N2CC[C@H](N3C4CCC3CC4)C2)n1] | 1.17 | 693 | A |
| Q-1ay | 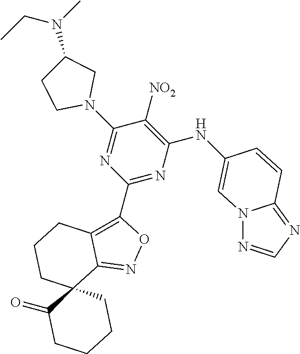 [SMILES: CCN(C)[C@H]1CCN(c2nc(-c3onc4c3CCC[C@@]43CCCCC3=O)nc(Nc3ccc4ncnn4c3)c2[N+](=O)[O-])C1] | 1.01 | 587 | A |
| Q-1az | 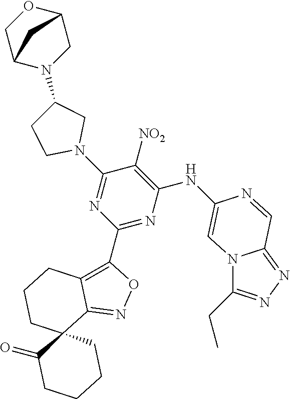 [SMILES: CCc1nnc2cnc(Nc3nc(-c4onc5c4CCC[C@@]54CCCCC4=O)nc(N4CC[C@H](N5C[C@@H]6C[C@H]5CO6)C4)c3[N+](=O)[O-])cn12] | 1.11 | 656 | A |
| Q-1ba | 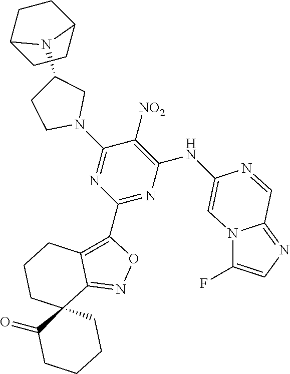 [SMILES: O=C1CCCC[C@@]12CCCc1c2noc1-c1nc(Nc2cn3c(F)cnc3cn2)c([N+](=O)[O-])c(N2CC[C@H](N3C4CCC3CC4)C2)n1] | 1.11 | 642 | A |
| Q-1bb | 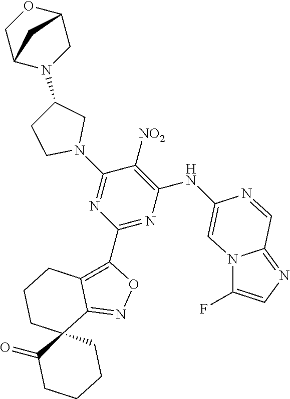 [SMILES: O=C1CCCC[C@@]12CCCc1c2noc1-c1nc(Nc2cn3c(F)cnc3cn2)c([N+](=O)[O-])c(N2CC[C@H](N3C[C@@H]4C[C@H]3CO4)C2)n1] | 1.13 | 644 | A |
| Q-1bc | 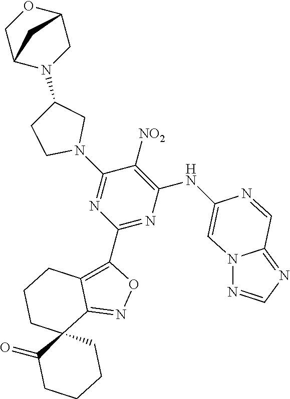 [SMILES: O=C1CCCC[C@@]12CCCc1c2noc1-c1nc(Nc2cn3ncnc3cn2)c([N+](=O)[O-])c(N2CC[C@H](N3C[C@@H]4C[C@H]3CO4)C2)n1] | 1.10 | 628 | A |
| Q-1bd | 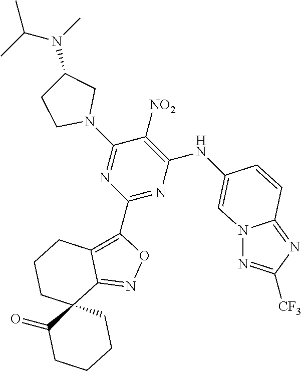 [SMILES: CC(C)N(C)[C@H]1CCN(c2nc(-c3onc4c3CCC[C@@]43CCCCC3=O)nc(Nc3ccc4nc(C(F)(F)F)nn4c3)c2[N+](=O)[O-])C1] | 1.13 | 669 | A |
| Q-1be | 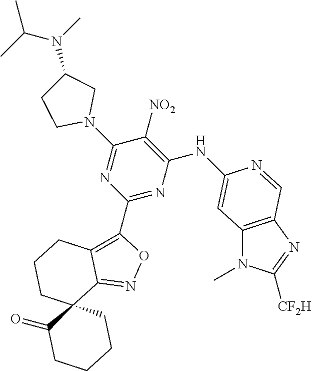 [SMILES: CC(C)N(C)[C@H]1CCN(c2nc(-c3onc4c3CCC[C@@]43CCCCC3=O)nc(Nc3cc4c(cn3)nc(C(F)F)n4C)c2[N+](=O)[O-])C1] | 1.14 | 666 | A |
| Q-1bf | 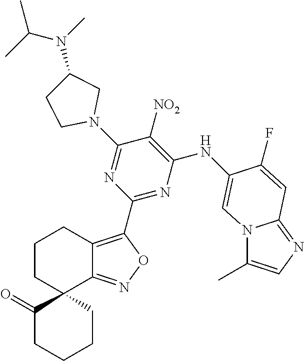 [SMILES: Cc1cnc2cc(F)c(Nc3nc(-c4onc5c4CCC[C@@]54CCCCC4=O)nc(N4CC[C@H](N(C)C(C)C)C4)c3[N+](=O)[O-])cn12] | 1.08 | 632 | A |
| Q-1bg | 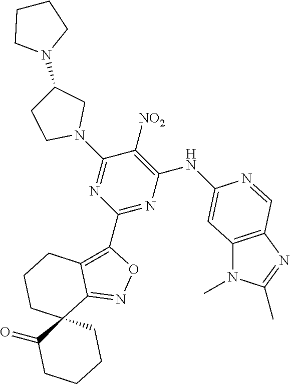 [SMILES: Cc1nc2cnc(Nc3nc(-c4onc5c4CCC[C@@]54CCCCC4=O)nc(N4CC[C@H](N5CCCC5)C4)c3[N+](=O)[O-])cc2n1C] | 0.97 | 601 | A |
| Q-1bh | 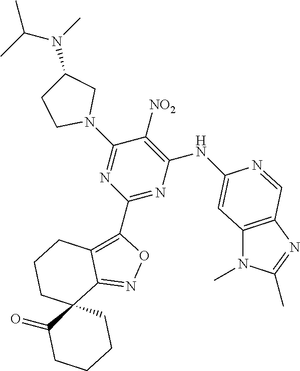 [SMILES: Cc1nc2cnc(Nc3nc(-c4onc5c4CCC[C@@]54CCCCC4=O)nc(N4CC[C@H](N(C)C(C)C)C4)c3[N+](=O)[O-])cc2n1C] | 1.05 | 629 | A |
| Q-1bi | 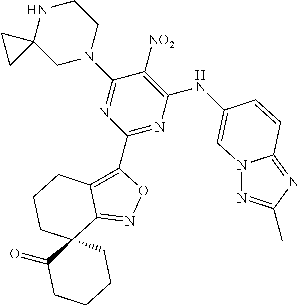 [SMILES: Cc1nc2ccc(Nc3nc(-c4onc5c4CCC[C@@]54CCCCC4=O)nc(N4CCNC5(CC5)C4)c3[N+](=O)[O-])cn2n1] | 0.95 | 623 | A |
| Q-1bj | 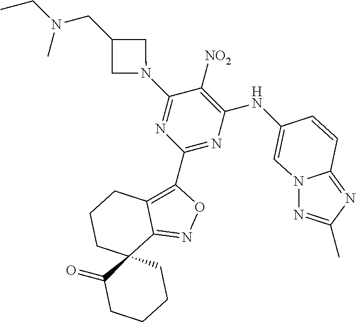 [SMILES: CCN(C)CC1CN(c2nc(-c3onc4c3CCC[C@@]43CCCCC3=O)nc(Nc3ccc4nc(C)nn4c3)c2[N+](=O)[O-])C1] | 1.03 | 601 | C |
| Q-1bk | 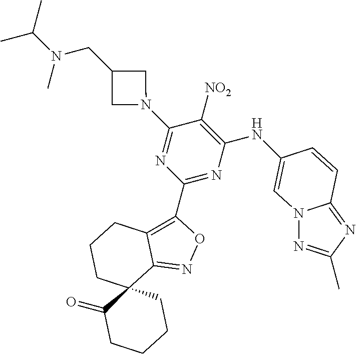 [SMILES: Cc1nc2ccc(Nc3nc(-c4onc5c4CCC[C@@]54CCCCC4=O)nc(N4CC(CN(C)C(C)C)C4)c3[N+](=O)[O-])cn2n1] | 1.04 | 615 | C |
| Q-1bl | 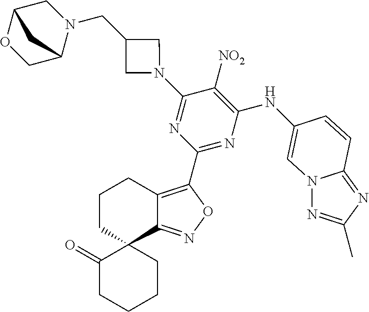 [SMILES: Cc1nc2ccc(Nc3nc(-c4onc5c4CCC[C@@]54CCCCC4=O)nc(N4CC(CN5C[C@@H]6C[C@H]5CO6)C4)c3[N+](=O)[O-])cn2n1] | 0.92 | 641 | C |
| Q-1bm | 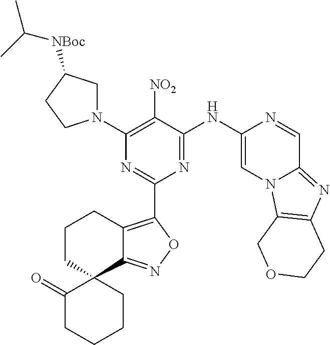 [SMILES: CC(C)N(C(=O)OC(C)(C)C)[C@H]1CCN(c2nc(-c3onc4c3CCC[C@@]43CCCCC3=O)nc(Nc3cn4c5c(nc4cn3)CCOC5)c2[N+](=O)[O-])C1] | 1.19 | 743 | C |
| Q-1bn | 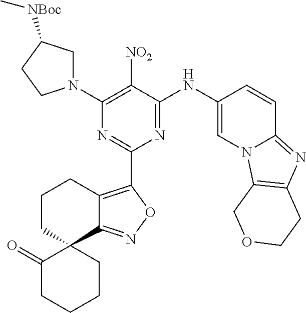 [SMILES: CN(C(=O)OC(C)(C)C)[C@H]1CCN(c2nc(-c3onc4c3CCC[C@@]43CCCCC3=O)nc(Nc3ccc4nc5c(n4c3)COCC5)c2[N+](=O)[O-])C1] | 1.10 | 714 | C |
| Q-1bo | 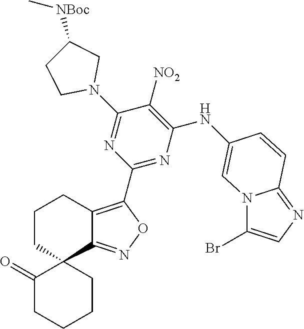 [SMILES: CN(C(=O)OC(C)(C)C)[C@H]1CCN(c2nc(-c3onc4c3CCC[C@@]43CCCCC3=O)nc(Nc3ccc4ncc(Br)n4c3)c2[N+](=O)[O-])C1] | 1.16 | 736 | C |
| Q-1bp | 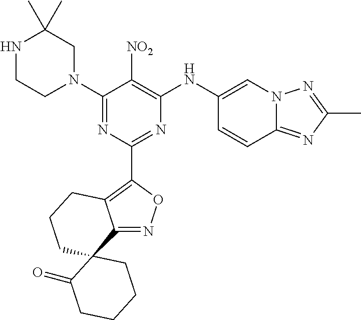 [SMILES: Cc1nc2ccc(Nc3nc(-c4onc5c4CCC[C@@]54CCCCC4=O)nc(N4CCNC(C)(C)C4)c3[N+](=O)[O-])cn2n1] | 0.94 | 587 | C |
| Q-1bq | 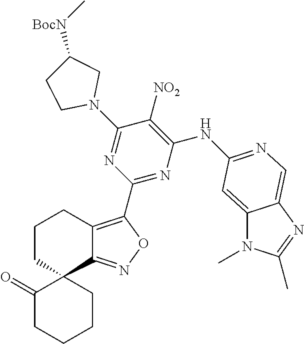 [SMILES: Cc1nc2cnc(Nc3nc(-c4onc5c4CCC[C@@]54CCCCC4=O)nc(N4CC[C@H](N(C)C(=O)OC(C)(C)C)C4)c3[N+](=O)[O-])cc2n1C] | 1.13 | 687 | C |
| Q-1br | 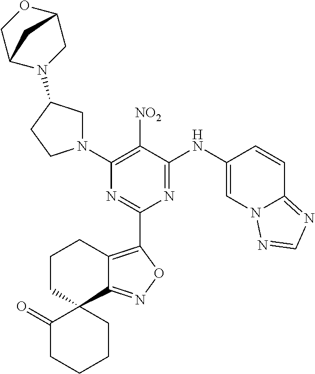 [SMILES: O=C1CCCC[C@@]12CCCc1c2noc1-c1nc(Nc2ccc3ncnn3c2)c([N+](=O)[O-])c(N2CC[C@H](N3C[C@@H]4C[C@H]3CO4)C2)n1] | 0.91 | 627 | C |
| Q-1bs | 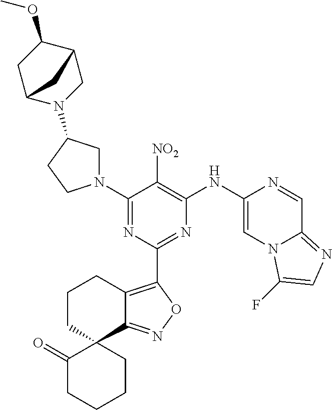 [SMILES: CO[C@@H]1C[C@@H]2C[C@H]1CN2[C@H]1CCN(c2nc(-c3onc4c3CCC[C@@]43CCCCC3=O)nc(Nc3cn4c(F)cnc4cn3)c2[N+](=O)[O-])C1] | 673 | 1.11 | C |
| Q-1bt | 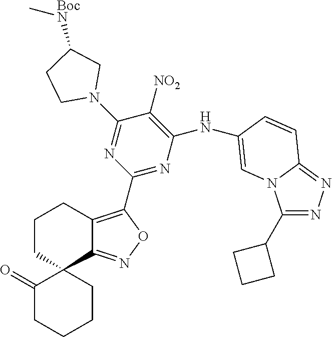 [SMILES: CN(C(=O)OC(C)(C)C)[C@H]1CCN(c2nc(-c3onc4c3CCC[C@@]43CCCCC3=O)nc(Nc3ccc4nnc(C5CCC5)n4c3)c2[N+](=O)[O-])C1] | 1.24 | 713 | C |
| Q-1bu | 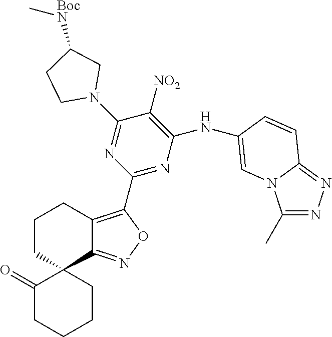 [SMILES: Cc1nnc2ccc(Nc3nc(-c4onc5c4CCC[C@@]54CCCCC4=O)nc(N4CC[C@H](N(C)C(=O)OC(C)(C)C)C4)c3[N+](=O)[O-])cn12] | 0.78 | 673 | C |
| Q-1bv | 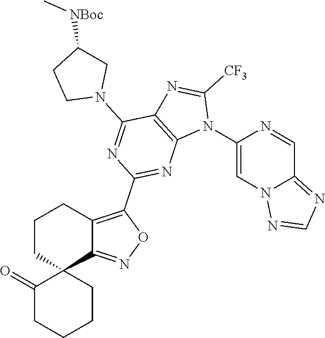 [SMILES: CN(C(=O)OC(C)(C)C)[C@H]1CCN(c2nc(-c3onc4c3CCC[C@@]43CCCCC3=O)nc3c2nc(C(F)(F)F)n3-c2cn3ncnc3cn2)C1] | 1.16 | 660 | A |
| Q-1bw | 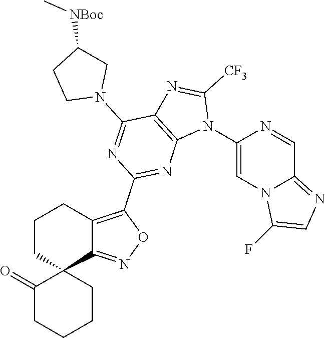 [SMILES: CN(C(=O)OC(C)(C)C)[C@H]1CCN(c2nc(-c3onc4c3CCC[C@@]43CCCCC3=O)nc3c2nc(C(F)(F)F)n3-c2cn3c(F)cnc3cn2)C1] | 1.19 | 677 | A |
| Q-1bx | 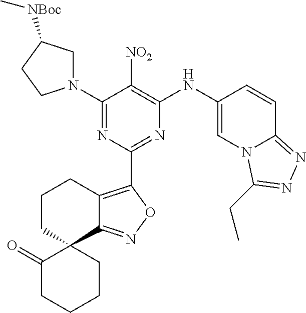 [SMILES: CCc1nnc2ccc(Nc3nc(-c4onc5c4CCC[C@@]54CCCCC4=O)nc(N4CC[C@H](N(C)C(=O)OC(C)(C)C)C4)c3[N+](=O)[O-])cn12] | 1.17 | 687 | A |
| Q-1by | 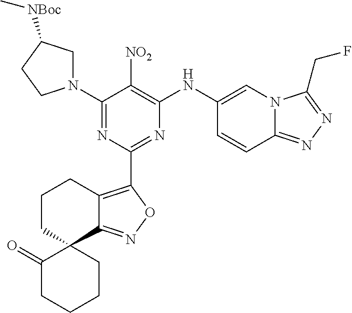 [SMILES: CN(C(=O)OC(C)(C)C)[C@H]1CCN(c2nc(-c3onc4c3CCC[C@@]43CCCCC3=O)nc(Nc3ccc4nnc(CF)n4c3)c2[N+](=O)[O-])C1] | 1.12 | 691 | A |
| Q-1bz | 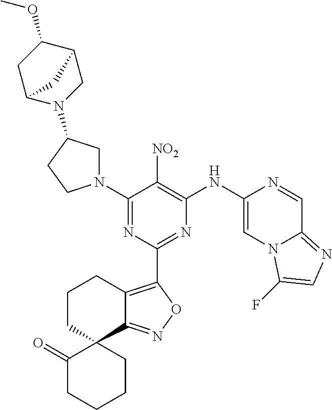 [SMILES: CO[C@H]1C[C@H]2C[C@@H]1CN2[C@H]1CCN(c2nc(-c3onc4c3CCC[C@@]43CCCCC3=O)nc(Nc3cn4c(F)cnc4cn3)c2[N+](=O)[O-])C1] | 1.05 | 673 | A |
| Q-1ca | 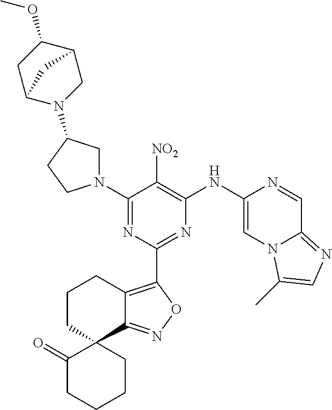 [SMILES: CO[C@H]1C[C@H]2C[C@@H]1CN2[C@H]1CCN(c2nc(-c3onc4c3CCC[C@@]43CCCCC3=O)nc(Nc3cn4c(C)cnc4cn3)c2[N+](=O)[O-])C1] | 1.07 | 669 | A |
| Q-1cb | 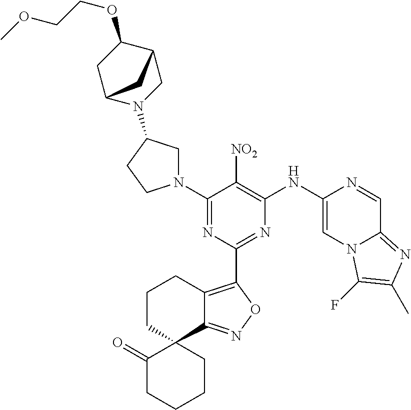 | 1.11 | 731 | A |
| Q-1cc | 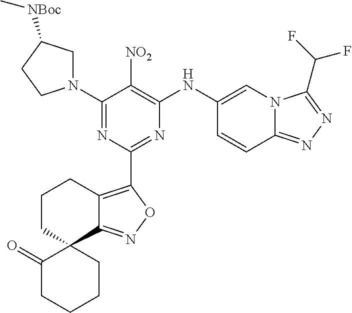 [SMILES: CN(C(=O)OC(C)(C)C)[C@H]1CCN(c2nc(-c3onc4c3CCC[C@@]43CCCCC3=O)nc(Nc3ccc4nnc(C(F)F)n4c3)c2[N+](=O)[O-])C1] | 1.20 | 709 | A |

### Synthesis of Intermediates D-1

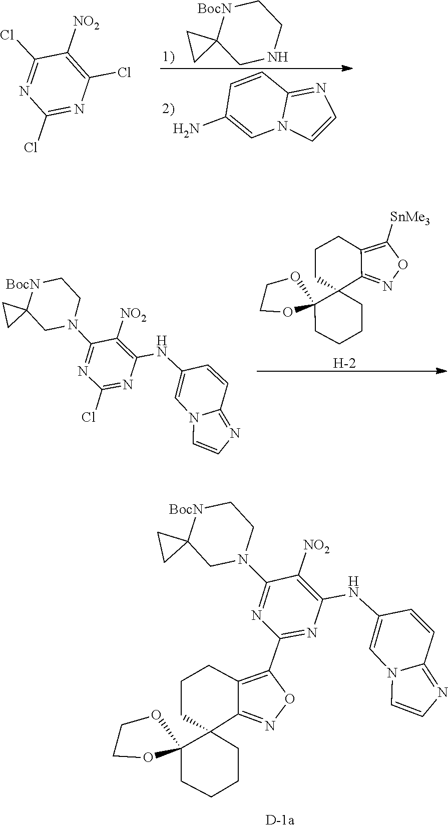

A solution of 2,4,6-trichloro-5-nitropyrimidine (3.00 g, 13.1 mmol, 1.0 equiv.) in DCM (20 mL) is cooled to −10° C. To this, a solution of tert-butyl 4,7-diazaspiro[2.5]octane-4-carboxylate (2.51 g, 11.8 mmol, 0.90 equiv.) and DIPEA (2.04 g, 15.8 mmol, 1.2 equiv.) in DCM (10 mL) is added slowly. The reaction mixture is stirred at 0° C. for 1 hour and then at room temperature for 2 hours. The reaction mixture is then diluted with additional DCM and extracted with an aqueous solution of ammonium chloride. The organic phase is dried with magnesium sulfate, filtered and the solvents are removed under reduced pressure. The crude monosubstituted nitropyrimidine (750 mg, 1.76 mmol, 1 equiv) was redissolved in THF (15 mL) and imidiazo-pyridine-6-amine (344 mg, 1.94 mmol, 1.1 equiv) was added and cooled to 0° C. LiHMDS solution in THF (3.8 mL, 3.8 mmol, 2.2 equiv) was added and the reaction mixture is stirred at room temperature for 4 h. The reaction mixture was diluted with DCM and aqeuous NaHCO₃ solution and extracted with DCM three times. The combined extracts were dried with Na₂SO₄ and evaporated to dryness.

The disubstituted pyrimidine (30 mg, 0.0587 mmol, 1.0 equiv.), (7S)-3-(trimethylstannyl)-5,6-dihydro-4H-dispiro[2,1-benzoxazole-7,1′-cyclohexane-2′,2″-[1,3]dioxolane]H-2 (34.18 mg, 0.0763 mmol, 1.3 equiv.), XPHOS Pd G4 (10.31 mg, 0.0117 mmol, 0.20 equiv.), and XPHOS (5.71 mg, 0.0117 mmol, 0.20 equiv.) is placed in a microwave vial. To this, 1,4-dioxane (1 mL) is added. The reaction mixture is flushed with argon for 5 minutes (in an ultrasound bath) and then heated to 120° C. in a microwave for 25 minutes.

The reaction mixture is then directly loaded onto an Isolute column and purified by reverse phase chromatography to give the desired product product D-1a (HPLC method A, tᵣₑₜ=1.19 min; [M+H]=714).

The following intermediate compounds D-1 (Table 18) are obtained analogously.

TABLE 18

| | | | | HPLC |
|---|---|---|---|---|
| # | Structure | tᵣₑₜ [min] | [M + H]⁺ | method |
| D-1b | 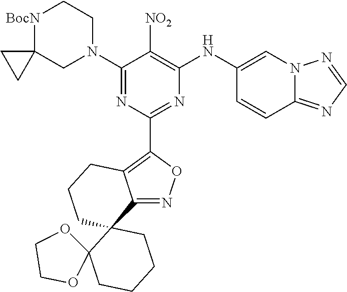 [SMILES: CC(C)(C)OC(=O)N1CCN(c2nc(-c3onc4c3CCC[C@@]43CCCCC34OCCO4)nc(Nc3ccc4ncnn4c3)c2[N+](=O)[O-])CC12CC2] | 1.17 | 685 | A |
| D-1c | 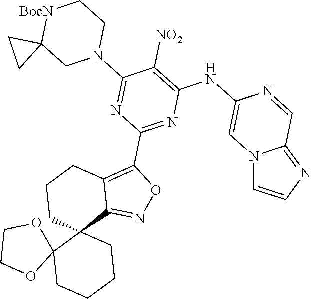 [SMILES: CC(C)(C)OC(=O)N1CCN(c2nc(-c3onc4c3CCC[C@@]43CCCCC34OCCO4)nc(Nc3cn4ccnc4cn3)c2[N+](=O)[O-])CC12CC2] | 1.22 | 744 | A |
| D-1d | 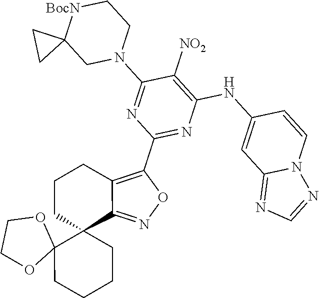 [SMILES: CC(C)(C)OC(=O)N1CCN(c2nc(-c3onc4c3CCC[C@@]43CCCCC34OCCO4)nc(Nc3ccn4ncnc4c3)c2[N+](=O)[O-])CC12CC2] | 1.22 | 771 | A |
| D-1e | 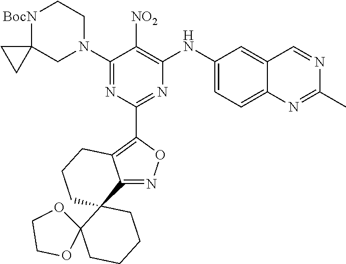 [SMILES: Cc1ncc2cc(Nc3nc(-c4onc5c4CCC[C@@]54CCCCC45OCCO5)nc(N4CCN(C(=O)OC(C)(C)C)C5(CC5)C4)c3[N+](=O)[O-])ccc2n1] | 1.22 | 740 | A |
| D-1f | 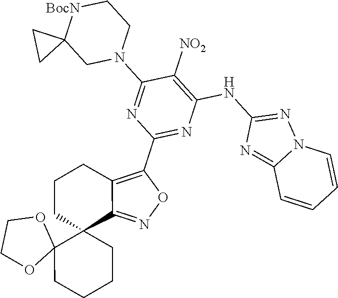 [SMILES: CC(C)(C)OC(=O)N1CCN(c2nc(-c3onc4c3CCC[C@@]43CCCCC34OCCO4)nc(Nc3nc4ccccn4n3)c2[N+](=O)[O-])CC12CC2] | 1.21 | 715 | A |
| D-1g | 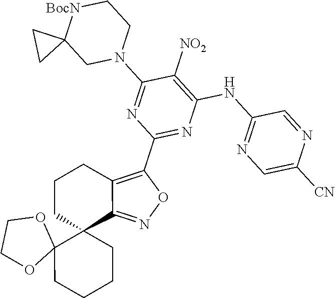 [SMILES: CC(C)(C)OC(=O)N1CCN(c2nc(-c3onc4c3CCC[C@@]43CCCCC34OCCO4)nc(Nc3cnc(C#N)cn3)c2[N+](=O)[O-])CC12CC2] | 1.26 | 701 | A |
| D-1h | 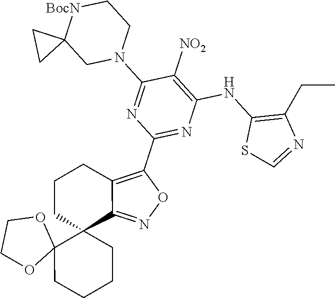 [SMILES: CCc1ncsc1Nc1nc(-c2onc3c2CCC[C@@]32CCCCC23OCCO3)nc(N2CCN(C(=O)OC(C)(C)C)C3(CC3)C2)c1[N+](=O)[O-]] | 1.27 | 709 | A |
| D-1i | 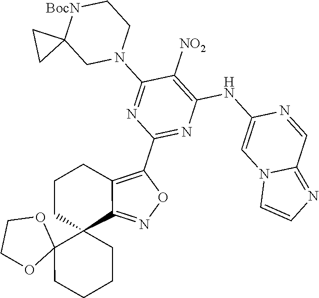 [SMILES: CC(C)(C)OC(=O)N1CCN(c2nc(-c3onc4c3CCC[C@@]43CCCCC34OCCO4)nc(Nc3cn4ccnc4cn3)c2[N+](=O)[O-])CC12CC2] | 1.22 | 715 | A |
| D-1j | 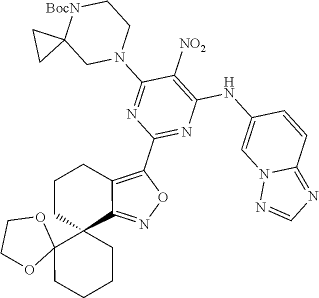 [SMILES: CC(C)(C)OC(=O)N1CCN(c2nc(-c3onc4c3CCC[C@@]43CCCCC34OCCO4)nc(Nc3ccc4ncnn4c3)c2[N+](=O)[O-])CC12CC2] | 1.22 | 715 | A |
| D-1k | 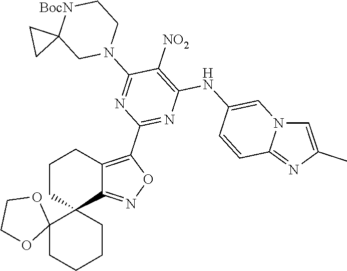 [SMILES: Cc1cn2cc(Nc3nc(-c4onc5c4CCC[C@@]54CCCCC45OCCO5)nc(N4CCN(C(=O)OC(C)(C)C)C5(CC5)C4)c3[N+](=O)[O-])ccc2n1] | 1.25 | 728 | A |
| D-1l | 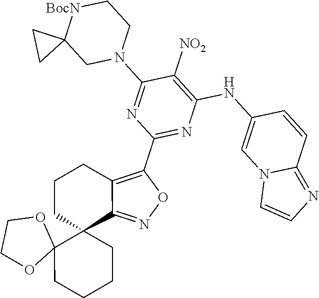 [SMILES: CC(C)(C)OC(=O)N1CCN(c2nc(-c3onc4c3CCC[C@@]43CCCCC34OCCO4)nc(Nc3ccc4nccn4c3)c2[N+](=O)[O-])CC12CC2] | 1.19 | 714 | A |
| D-1m | 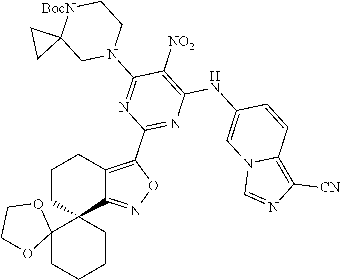 [SMILES: CC(C)(C)OC(=O)N1CCN(c2nc(-c3onc4c3CCC[C@@]43CCCCC34OCCO4)nc(Nc3ccc4c(C#N)ncn4c3)c2[N+](=O)[O-])CC12CC2] | 1.19 | 739 | A |
| D-1n | 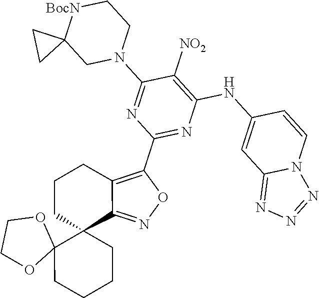 [SMILES: CC(C)(C)OC(=O)N1CCN(c2nc(-c3onc4c3CCC[C@@]43CCCCC34OCCO4)nc(Nc3ccn4nnnc4c3)c2[N+](=O)[O-])CC12CC2] | 1.25 | 716 | A |
| D-1o | 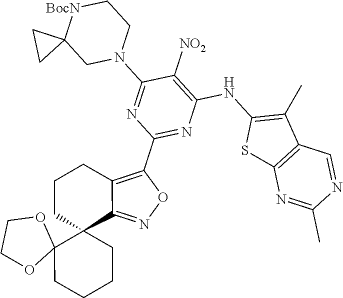 [SMILES: Cc1ncc2c(C)c(Nc3nc(-c4onc5c4CCC[C@@]54CCCCC45OCCO5)nc(N4CCN(C(=O)OC(C)(C)C)C5(CC5)C4)c3[N+](=O)[O-])sc2n1] | 1.29 | 760 | A |
| D-1p | 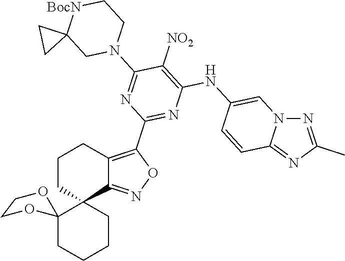 [SMILES: Cc1nc2ccc(Nc3nc(-c4onc5c4CCC[C@@]54CCCCC45OCCO5)nc(N4CCN(C(=O)OC(C)(C)C)C5(CC5)C4)c3[N+](=O)[O-])cn2n1] | 1.23 | 729 | C |
| D-1q | 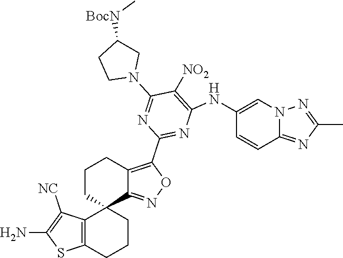 [SMILES: Cc1nc2ccc(Nc3nc(-c4onc5c4CCC[C@@]54CCCc5sc(N)c(C#N)c54)nc(N4CC[C@H](N(C)C(=O)OC(C)(C)C)C4)c3[N+](=O)[O-])cn2n1] | 2.49 | 717 | AB |
| D-1r | 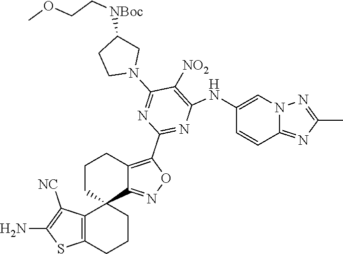 [SMILES: COCCN(C(=O)OC(C)(C)C)[C@H]1CCN(c2nc(-c3onc4c3CCC[C@@]43CCCc4sc(N)c(C#N)c43)nc(Nc3ccc4nc(C)nn4c3)c2[N+](=O)[O-])C1] | 1.26 | 761 | A |

### Synthesis of Purine Intermediates D-3

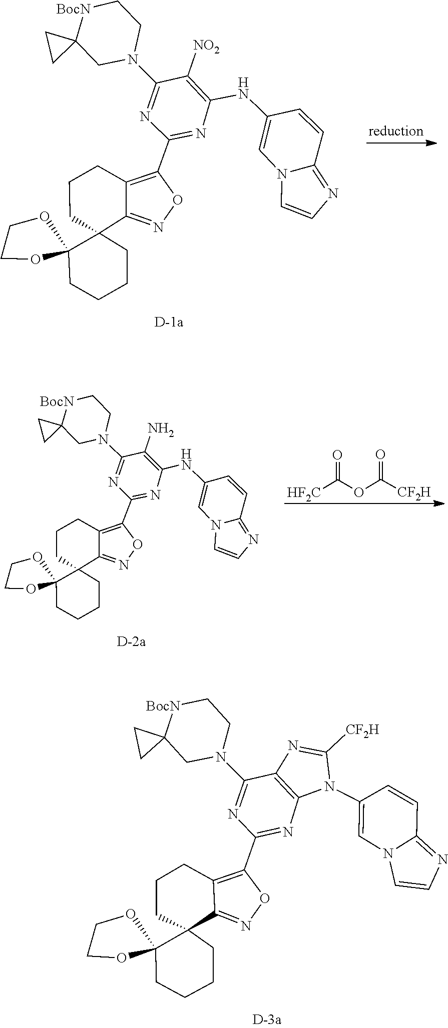

D-1a (152 mg, 0.21 mmol, 1 equiv) and Pd/C 10% (50 mg) are suspended in THF (15 mL) and stirred under hydrogen pressure (6 bar) for 1 hour at room temperature. The reaction mixture is filtered over celite and evaporated to dryness. D-2a is directly used in the next step without purification.

D-2a (59 mg, 0.086 mmol, 1 equiv) was dissolved in CH₂Cl₂ (1 mL) and cooled to −20° C. Triethyl amine (0.04 mL, 0.17 mmol, 3 equiv) and difluoroacetic anhydride (0.02 mL, 0.173 mmol, 2 equiv) were added sequentially and the reaction mixture is stirred for 20 min at −20° C. The mixture was quenched with water and then extracted with CH₂Cl₂ three times. The combined extracts were dried with Na₂SO₄ and evaporated to dryness. The crude product was purified by RP-chromatography to give the desired product product D-3a (HPLC method A, tᵣₑₜ=1.19 min; [M+H]=744).

The following intermediate compounds D-3 (Table 19) are obtained analogously.

TABLE 19

| | | | | HPLC |
|---|---|---|---|---|
| # | Structure | tᵣₑₜ [min] | [M + H]⁺ | method |
| D-3b | 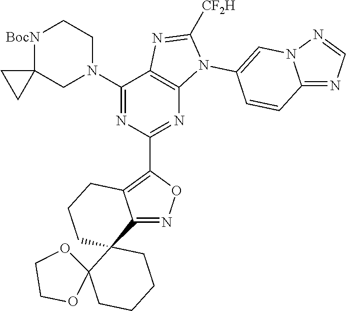 [SMILES: CC(C)(C)OC(=O)N1CCN(c2nc(-c3onc4c3CCC[C@@]43CCCCC34OCCO4)nc3c2nc(C(F)F)n3-c2ccc3ncnn3c2)CC12CC2] | 1.25 | 745 | A |
| D-3c | 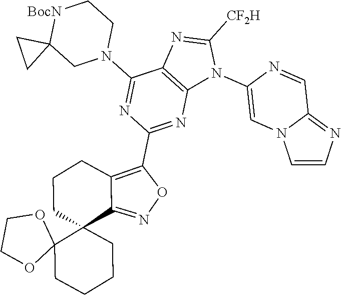 [SMILES: CC(C)(C)OC(=O)N1CCN(c2nc(-c3onc4c3CCC[C@@]43CCCCC34OCCO4)nc3c2nc(C(F)F)n3-c2cn3ccnc3cn2)CC12CC2] | 1.22 | 744 | A |
| D-3d | 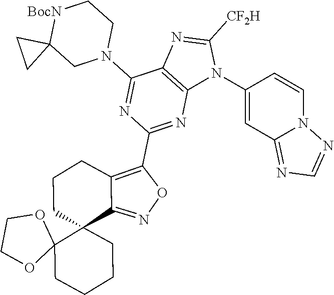 [SMILES: CC(C)(C)OC(=O)N1CCN(c2nc(-c3onc4c3CCC[C@@]43CCCCC34OCCO4)nc3c2nc(C(F)F)n3-c2ccn3ncnc3c2)CC12CC2] | 1.23 | 744 | A |
| D-3e | 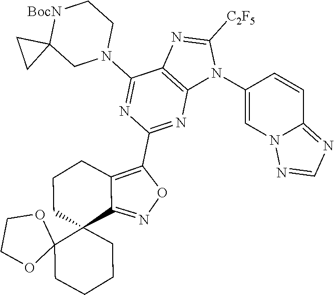 | 1.27 | 813 | A |
| D-3f | 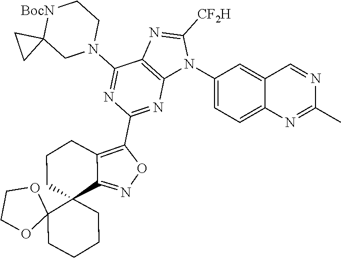 [SMILES: Cc1ncc2cc(-n3c(C(F)F)nc4c(N5CCN(C(=O)OC(C)(C)C)C6(CC6)C5)nc(-c5onc6c5CCC[C@@]65CCCCC56OCCO6)nc43)ccc2n1] | 1.27 | 770 | A |
| D-3g | 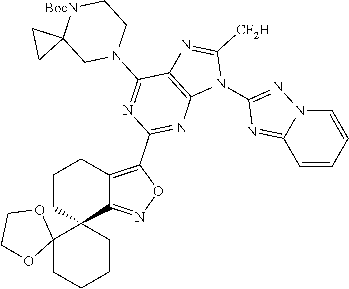 [SMILES: CC(C)(C)OC(=O)N1CCN(c2nc(-c3onc4c3CCC[C@@]43CCCCC34OCCO4)nc3c2nc(C(F)F)n3-c2nc3ccccn3n2)CC12CC2] | 1.30 | 745 | A |
| D-3h | 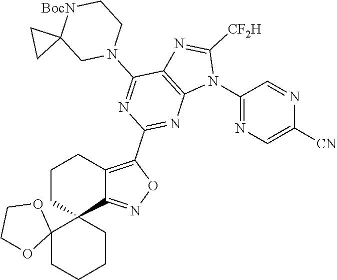 [SMILES: CC(C)(C)OC(=O)N1CCN(c2nc(-c3onc4c3CCC[C@@]43CCCCC34OCCO4)nc3c2nc(C(F)F)n3-c2cnc(C#N)cn2)CC12CC2] | 1.28 | 731 | A |
| D-3i | 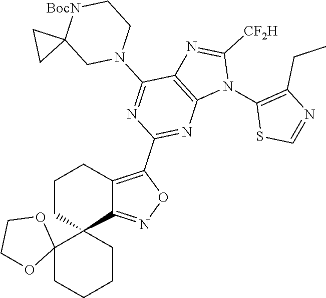 [SMILES: CCc1ncsc1-n1c(C(F)F)nc2c(N3CCN(C(=O)OC(C)(C)C)C4(CC4)C3)nc(-c3onc4c3CCC[C@@]43CCCCC34OCCO4)nc21] | 1.29 | 739 | A |
| D-3j | 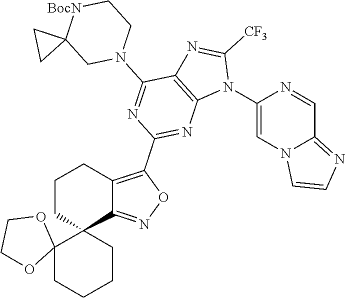 [SMILES: CC(C)(C)OC(=O)N1CCN(c2nc(-c3onc4c3CCC[C@@]43CCCCC34OCCO4)nc3c2nc(C(F)(F)F)n3-c2cn3ccnc3cn2)CC12CC2] | 1.22 | 763 | A |
| D-3k | 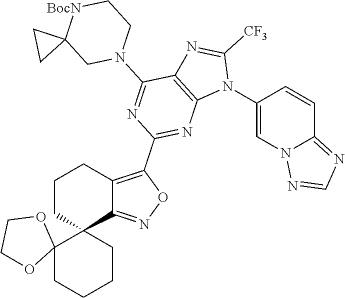 [SMILES: CC(C)(C)OC(=O)N1CCN(c2nc(-c3onc4c3CCC[C@@]43CCCCC34OCCO4)nc3c2nc(C(F)(F)F)n3-c2ccc3ncnn3c2)CC12CC2] | 1.24 | 763 | A |
| D-3l | 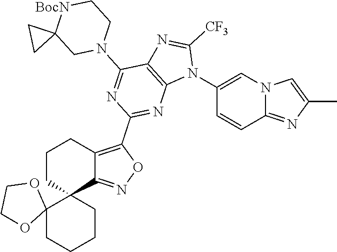 [SMILES: Cc1cn2cc(-n3c(C(F)(F)F)nc4c(N5CCN(C(=O)OC(C)(C)C)C6(CC6)C5)nc(-c5onc6c5CCC[C@@]65CCCCC56OCCO6)nc43)ccc2n1] | 1.19 | 776 | A |
| D-3m | 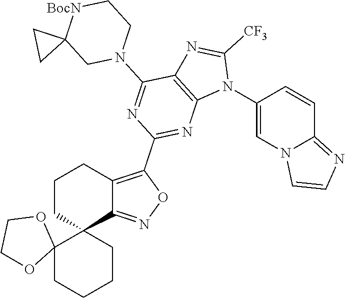 [SMILES: CC(C)(C)OC(=O)N1CCN(c2nc(-c3onc4c3CCC[C@@]43CCCCC34OCCO4)nc3c2nc(C(F)(F)F)n3-c2ccc3nccn3c2)CC12CC2] | 1.22 | 762 | A |
| D-3n | 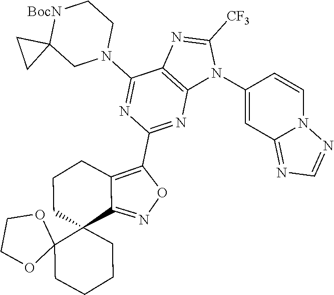 [SMILES: CC(C)(C)OC(=O)N1CCN(c2nc(-c3onc4c3CCC[C@@]43CCCCC34OCCO4)nc3c2nc(C(F)(F)F)n3-c2ccn3ncnc3c2)CC12CC2] | 1.25 | 763 | A |
| D-3o | 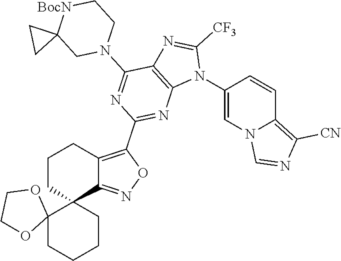 [SMILES: CC(C)(C)OC(=O)N1CCN(c2nc(-c3onc4c3CCC[C@@]43CCCCC34OCCO4)nc3c2nc(C(F)(F)F)n3-c2ccc3c(C#N)ncn3c2)CC12CC2] | 1.26 | 787 | A |
| D-3p | 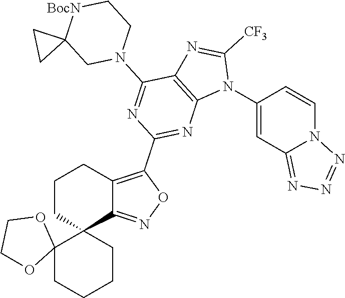 [SMILES: CC(C)(C)OC(=O)N1CCN(c2nc(-c3onc4c3CCC[C@@]43CCCCC34OCCO4)nc3c2nc(C(F)(F)F)n3-c2ccn3nnnc3c2)CC12CC2] | 1.28 | 764 | A |
| D-3q | 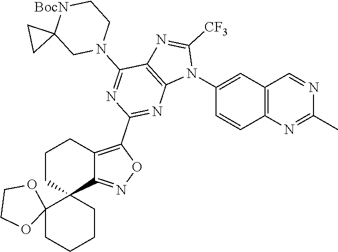 [SMILES: Cc1ncc2cc(-n3c(C(F)(F)F)nc4c(N5CCN(C(=O)OC(C)(C)C)C6(CC6)C5)nc(-c5onc6c5CCC[C@@]65CCCCC56OCCO6)nc43)ccc2n1] | 1.32 | 788 | A |
| D-3r | 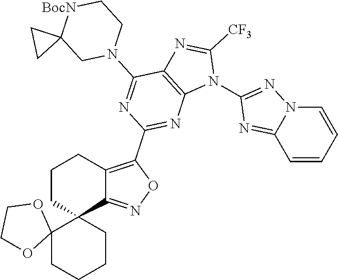 [SMILES: CC(C)(C)OC(=O)N1CCN(c2nc(-c3onc4c3CCC[C@@]43CCCCC34OCCO4)nc3c2nc(C(F)(F)F)n3-c2nc3ccccn3n2)CC12CC2] | 1.28 | 763 | A |
| D-3s | 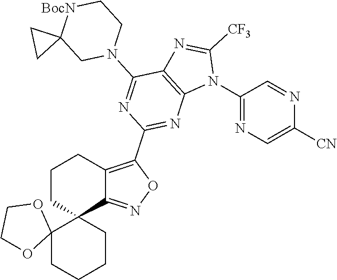 [SMILES: CC(C)(C)OC(=O)N1CCN(c2nc(-c3onc4c3CCC[C@@]43CCCCC34OCCO4)nc3c2nc(C(F)(F)F)n3-c2cnc(C#N)cn2)CC12CC2] | 1.31 | 749 | A |
| D-3t | 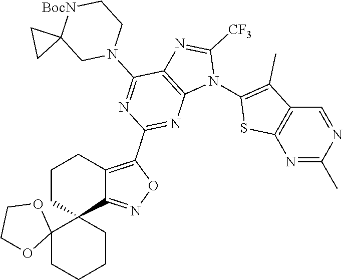 [SMILES: Cc1ncc2c(C)c(-n3c(C(F)(F)F)nc4c(N5CCN(C(=O)OC(C)(C)C)C6(CC6)C5)nc(-c5onc6c5CCC[C@@]65CCCCC56OCCO6)nc43)sc2n1] | 1.37 | 808 | A |
| D-3u | 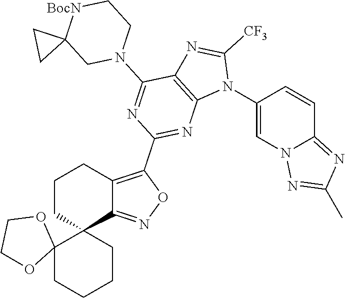 [SMILES: Cc1nc2ccc(-n3c(C(F)(F)F)nc4c(N5CCN(C(=O)OC(C)(C)C)C6(CC6)C5)nc(-c5onc6c5CCC[C@@]65CCCCC56OCCO6)nc43)cn2n1] | 1.26 | 777 | C |
| D-3v | 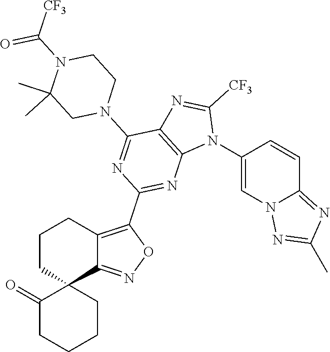 [SMILES: Cc1nc2ccc(-n3c(C(F)(F)F)nc4c(N5CCN(C(=O)C(F)(F)F)C(C)(C)C5)nc(-c5onc6c5CCC[C@@]65CCCCC5=O)nc43)cn2n1] | 1.13 | 731 | C |
| D-3w | 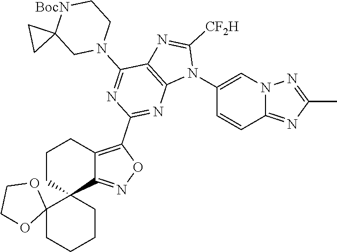 [SMILES: Cc1nc2ccc(-n3c(C(F)F)nc4c(N5CCN(C(=O)OC(C)(C)C)C6(CC6)C5)nc(-c5onc6c5CCC[C@@]65CCCCC56OCCO6)nc43)cn2n1] | 1.26 | 759 | C |

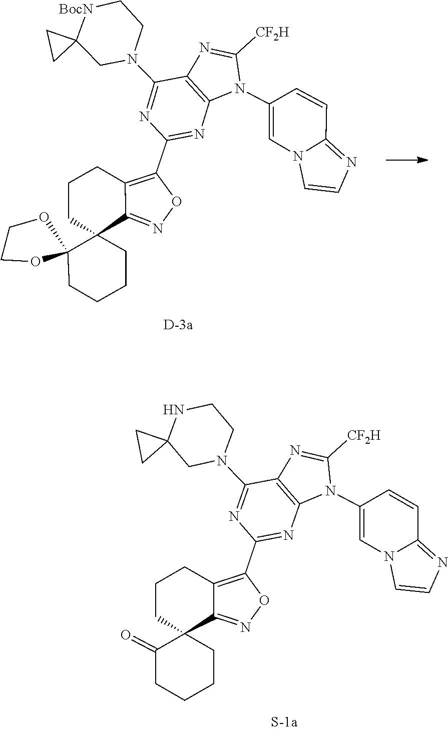

D-3a (64 mg, 0.086 mmol, 1.0 equiv) is dissolved in dioxane (5 mL), followed by addition of 6 M HCl in dioxane (0.58 mL, 3.5 mmol, 40 equiv). The reaction mixture is stirred for 30 min at room temperature, before it is quenched with NaHCO₃ solution. The suspension is filtered over celite and extracted with CH₂Cl₂ three times. The combined extracts are dried with Na₂SO₄ and evaporated to dryness. The crude product is purified by RP-chromatography to give the desired product S-1a (HPLC method A, tᵣₑₜ=0.87 min; [M+H]=600).

The following intermediate compounds S-1 (Table 20) are obtained analogously.

TABLE 20

| | | | | HPLC |
|---|---|---|---|---|
| # | Structure | tᵣₑₜ [min] | [M + H]⁺ | method |
| S-1b | 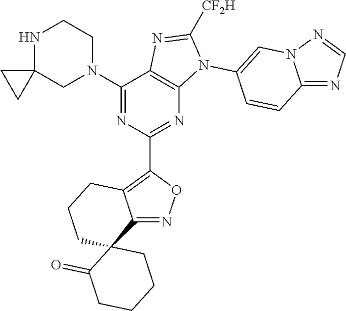 [SMILES: O=C1CCCC[C@@]12CCCc1c2noc1-c1nc(N2CCNC3(CC3)C2)c2nc(C(F)F)n(-c3ccc4ncnn4c3)c2n1] | 1.29 | 681 | A |
| S-1c | 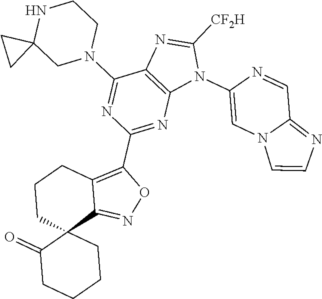 [SMILES: O=C1CCCC[C@@]12CCCc1c2noc1-c1nc(N2CCNC3(CC3)C2)c2nc(C(F)F)n(-c3cn4ccnc4cn3)c2n1] | 0.93 | 619 | A |
| S-1d | 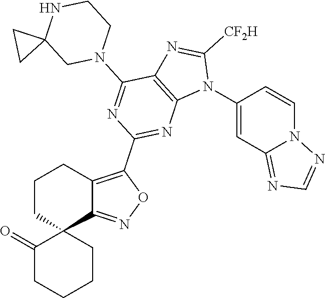 [SMILES: O=C1CCCC[C@@]12CCCc1c2noc1-c1nc(N2CCNC3(CC3)C2)c2nc(C(F)F)n(-c3ccn4ncnc4c3)c2n1] | 0.94 | 601 | A |
| S-1e | 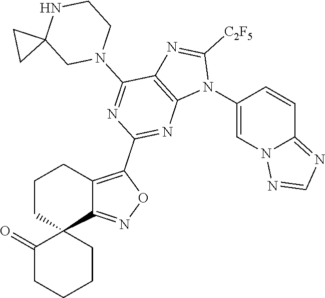 | 0.72 | 669 | A |
| S-1f | 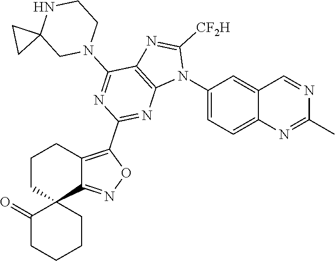 [SMILES: Cc1ncc2cc(-n3c(C(F)F)nc4c(N5CCNC6(CC6)C5)nc(-c5onc6c5CCC[C@@]65CCCCC5=O)nc43)ccc2n1] | 1.02 | 587 | A |
| S-1g | 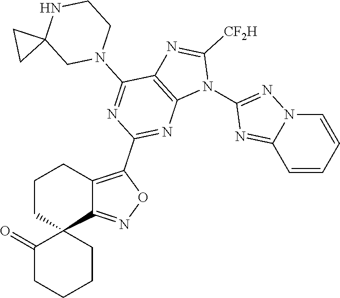 [SMILES: O=C1CCCC[C@@]12CCCc1c2noc1-c1nc(N2CCNC3(CC3)C2)c2nc(C(F)F)n(-c3nc4ccccn4n3)c2n1] | 0.98 | 601 | A |
| S-1h | 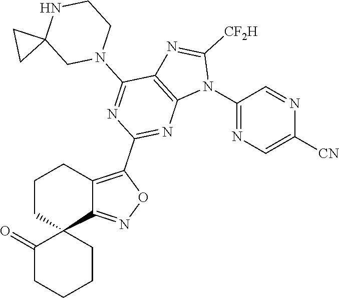 [SMILES: N#Cc1cnc(-n2c(C(F)F)nc3c(N4CCNC5(CC5)C4)nc(-c4onc5c4CCC[C@@]54CCCCC4=O)nc32)cn1] | 1.02 | 587 | A |
| S-1i | 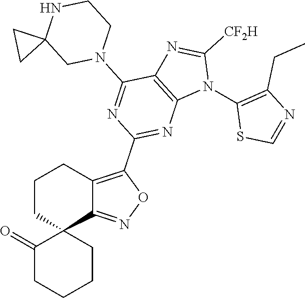 [SMILES: CCc1ncsc1-n1c(C(F)F)nc2c(N3CCNC4(CC4)C3)nc(-c3onc4c3CCC[C@@]43CCCCC3=O)nc21] | 1.01 | 595 | A |
| S-1k | 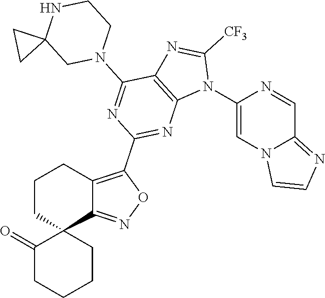 [SMILES: O=C1CCCC[C@@]12CCCc1c2noc1-c1nc(N2CCNC3(CC3)C2)c2nc(C(F)(F)F)n(-c3cn4ccnc4cn3)c2n1] | 0.94 | 619 | A |
| S-1l | 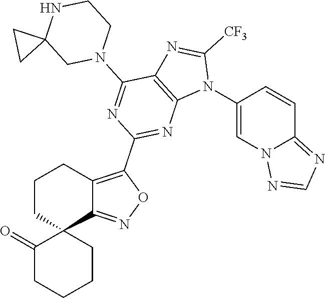 [SMILES: O=C1CCCC[C@@]12CCCc1c2noc1-c1nc(N2CCNC3(CC3)C2)c2nc(C(F)(F)F)n(-c3ccc4ncnn4c3)c2n1] | 0.94 | 619 | A |
| S-1m | 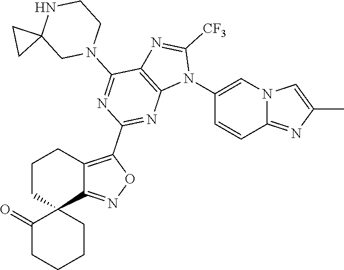 [SMILES: Cc1cn2cc(-n3c(C(F)(F)F)nc4c(N5CCNC6(CC6)C5)nc(-c5onc6c5CCC[C@@]65CCCCC5=O)nc43)ccc2n1] | 1.12 | 631 | A |
| S-1n | 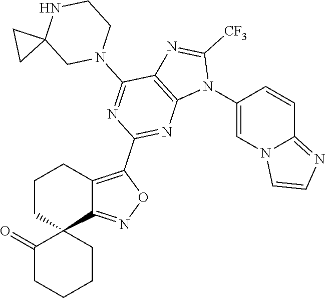 [SMILES: O=C1CCCC[C@@]12CCCc1c2noc1-c1nc(N2CCNC3(CC3)C2)c2nc(C(F)(F)F)n(-c3ccc4nccn4c3)c2n1] | 0.92 | 617 | A |
| S-1o | 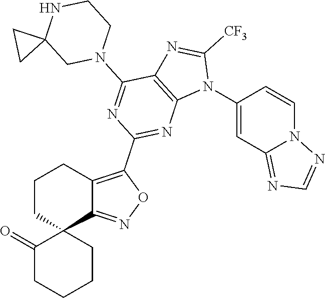 [SMILES: O=C1CCCC[C@@]12CCCc1c2noc1-c1nc(N2CCNC3(CC3)C2)c2nc(C(F)(F)F)n(-c3ccn4ncnc4c3)c2n1] | 0.96 | 613 | A |
| S-1p | 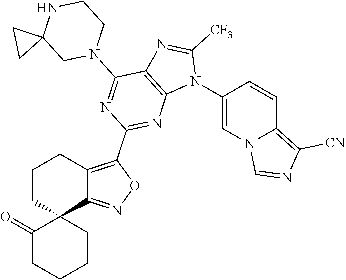 [SMILES: N#Cc1ncn2cc(-n3c(C(F)(F)F)nc4c(N5CCNC6(CC6)C5)nc(-c5onc6c5CCC[C@@]65CCCCC5=O)nc43)ccc12] | 1.01 | 643 | A |
| S-1q | 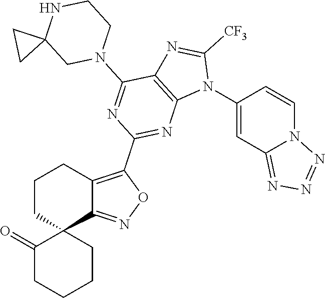 [SMILES: O=C1CCCC[C@@]12CCCc1c2noc1-c1nc(N2CCNC3(CC3)C2)c2nc(C(F)(F)F)n(-c3ccn4nnnc4c3)c2n1] | 1.03 | 620 | A |
| S-1r | 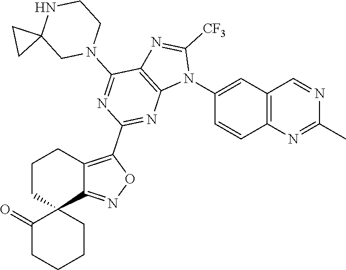 [SMILES: Cc1ncc2cc(-n3c(C(F)(F)F)nc4c(N5CCNC6(CC6)C5)nc(-c5onc6c5CCC[C@@]65CCCCC5=O)nc43)ccc2n1] | 1.00 | 644 | A |
| S-1s | 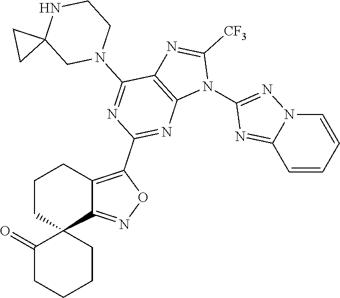 [SMILES: O=C1CCCC[C@@]12CCCc1c2noc1-c1nc(N2CCNC3(CC3)C2)c2nc(C(F)(F)F)n(-c3nc4ccccn4n3)c2n1] | 1.02 | 620 | A |
| S-1t | 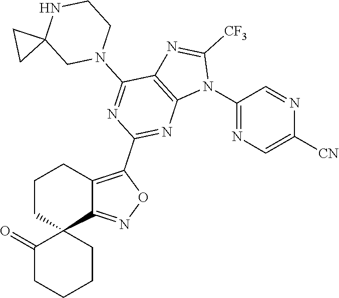 [SMILES: N#Cc1cnc(-n2c(C(F)(F)F)nc3c(N4CCNC5(CC5)C4)nc(-c4onc5c4CCC[C@@]54CCCCC4=O)nc32)cn1] | 1.06 | 605 | A |
| S-1u | 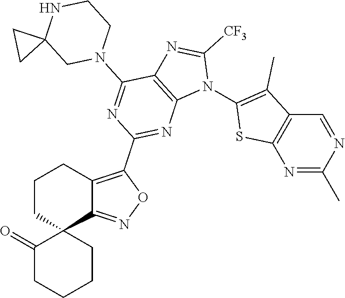 [SMILES: Cc1ncc2c(C)c(-n3c(C(F)(F)F)nc4c(N5CCNC6(CC6)C5)nc(-c5onc6c5CCC[C@@]65CCCCC5=O)nc43)sc2n1] | 1.08 | 664 | A |
| S-1v | 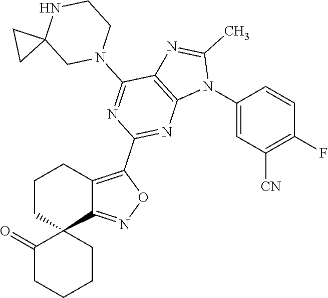 [SMILES: Cc1nc2c(N3CCNC4(CC4)C3)nc(-c3onc4c3CCC[C@@]43CCCCC3=O)nc2n1-c1ccc(F)c(C#N)c1] | 1.12 | 567 | A |
| S-1w | 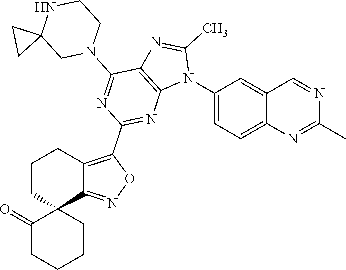 [SMILES: Cc1ncc2cc(-n3c(C)nc4c(N5CCNC6(CC6)C5)nc(-c5onc6c5CCC[C@@]65CCCCC5=O)nc43)ccc2n1] | 1.26 | 590 | A |
| S-1x | 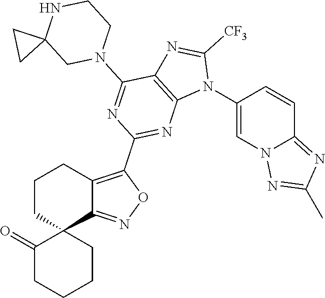 [SMILES: Cc1nc2ccc(-n3c(C(F)(F)F)nc4c(N5CCNC6(CC6)C5)nc(-c5onc6c5CCC[C@@]65CCCCC5=O)nc43)cn2n1] | 0.97 | 633 | C |
| S-1y | 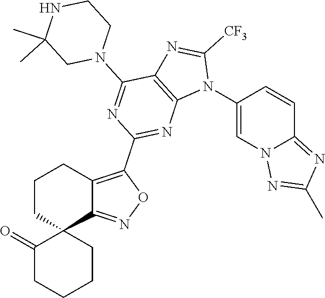 [SMILES: Cc1nc2ccc(-n3c(C(F)(F)F)nc4c(N5CCNC(C)(C)C5)nc(-c5onc6c5CCC[C@@]65CCCCC5=O)nc43)cn2n1] | 0.99 | 635 | C |
| S-1z | 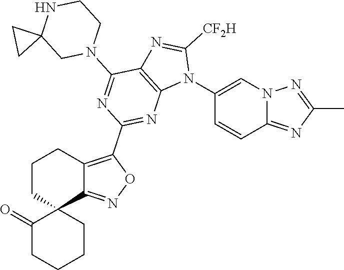 [SMILES: Cc1nc2ccc(-n3c(C(F)F)nc4c(N5CCNC6(CC6)C5)nc(-c5onc6c5CCC[C@@]65CCCCC5=O)nc43)cn2n1] | 1.00 | 615 | A |

### Reductive Purine Formation

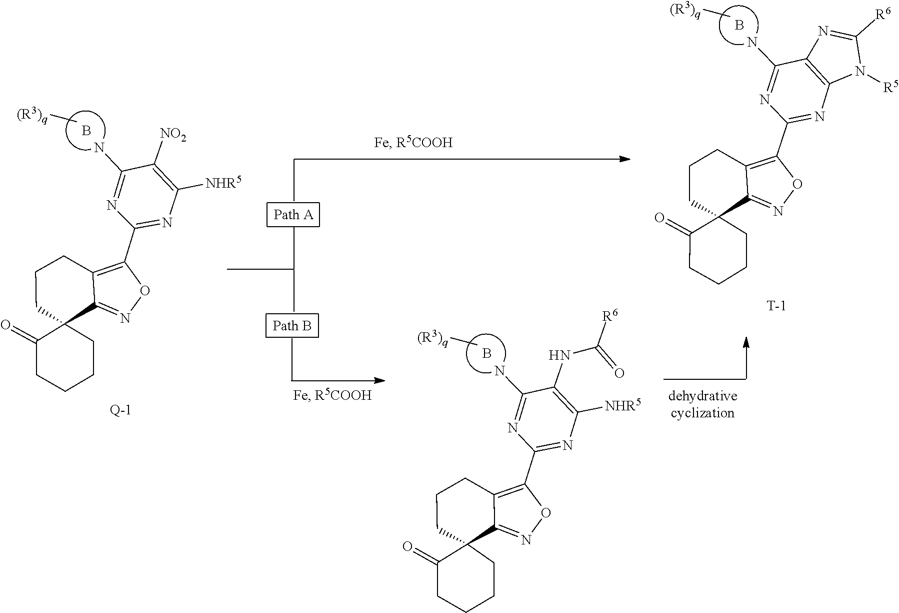

### General Procedure Path A

The Q-1 was dissolved in trifluoroacetic acid (0.13 M), followed by addition of iron powder (5.0 equiv). The reaction mixture was stirred for 16 h at 60° C. If reaction monitoring with HPLC-MS indicates full conversion, the reaction mixture was diluted with CH₂Cl₂ and aqeuous NaHCO₃ solution was added carefully until gas evolution ceased. The suspension was filtered over celite and extracted with CH₂Cl₂ three times. The combined extracts were dried with Na₂SO₄ and evaporated to dryness. The crude product was purified by RP-chromatography to give the desired product T-1.

### General Procedure Path B

The Q-1 was dissolved in trifluoroacetic acid (0.13 M), followed by addition of iron powder (5.0 equiv). The reaction mixture was stirred for 16 h at 60° C. If reaction monitoring with HPLC-MS indicates full conversion, the reaction mixture was diluted with CH₂Cl₂ and aqeuous NaHCO₃ solution was added carefully until gas evolution ceased. The suspension was filtered over celite and extracted with CH₂Cl₂ three times. The crude product was dissolved in dichloro ethane (0.2 M) and N,O-bis(trimethylsilyl)acetamide (12 equiv) was added. The reaction mixture was stirred for 1 h at 90° C. If reaction monitoring with HPLC-MS indicates full conversion, the reaction mixture was diluted with CH₂Cl₂ and aqeuous NaHCO₃ solution was added carefully until gas evolution ceased. The suspension was filtered over celite and extracted with CH₂Cl₂ three times. The combined extracts were dried with Na₂SO₄ and evaporated to dryness. The crude product was purified by RP-chromatography to give the desired product T-1.

The following intermediate products T-1 (Table 21) are obtained by applying the indicated general procedures.

TABLE 21

| | | tᵣₑₜ | | HPLC | General |
|---|---|---|---|---|---|
| # | structure | [min] | [M + H]⁺ | method | Procedure |
| T-1a | 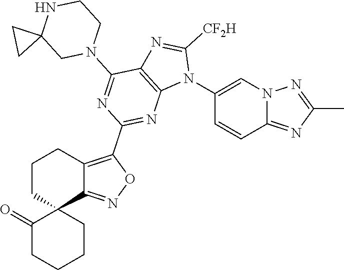 [SMILES: Cc1nc2ccc(-n3c(C(F)F)nc4c(N5CCNC6(CC6)C5)nc(-c5onc6c5CCC[C@@]65CCCCC5=O)nc43)cn2n1] | 1.00 | 615 | A | B |
| T-1b | 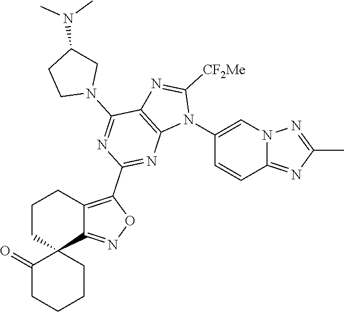 | 1.12 | 631 | A | B |
| T-1c | 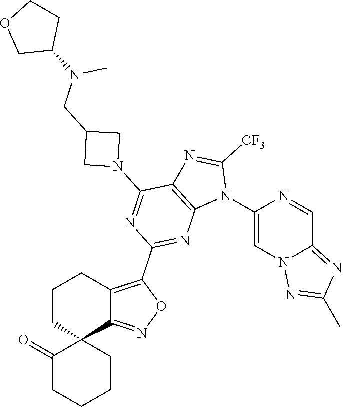 [SMILES: Cc1nc2cnc(-n3c(C(F)(F)F)nc4c(N5CC(CN(C)[C@H]6CCOC6)C5)nc(-c5onc6c5CCC[C@@]65CCCCC5=O)nc43)cn2n1] | 0.96 | 692 | A | B |
| T-1d | 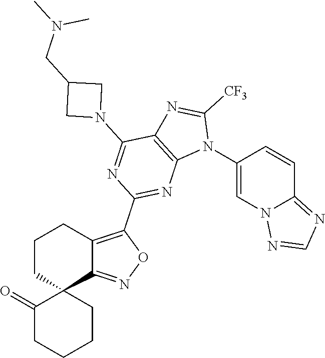 [SMILES: CN(C)CC1CN(c2nc(-c3onc4c3CCC[C@@]43CCCCC3=O)nc3c2nc(C(F)(F)F)n3-c2ccc3ncnn3c2)C1] | 0.94 | 621 | A | B |
| T-1e | 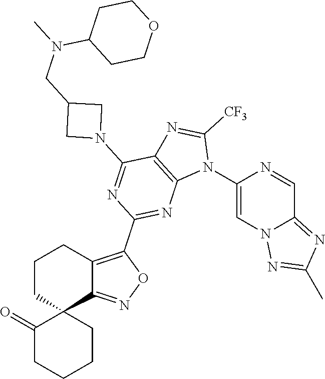 [SMILES: Cc1nc2cnc(-n3c(C(F)(F)F)nc4c(N5CC(CN(C)C6CCOCC6)C5)nc(-c5onc6c5CCC[C@@]65CCCCC5=O)nc43)cn2n1] | 0.99 | 706 | A | B |
| T-1f | 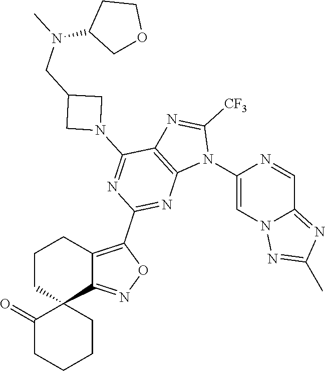 | 0.97 | 692 | A | B |
| T-1g | 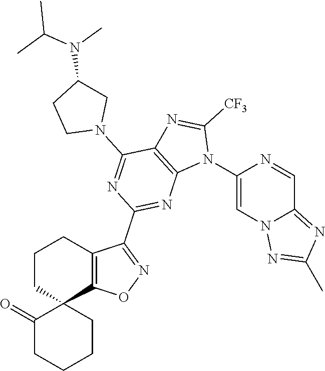 [SMILES: Cc1nc2cnc(-n3c(C(F)(F)F)nc4c(N5CC[C@H](N(C)C(C)C)C5)nc(-c5noc6c5CCC[C@@]65CCCCC5=O)nc43)cn2n1] | 1.11 | 664 | A | B |
| T-1h | 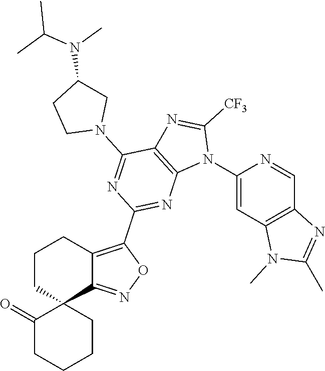 [SMILES: Cc1nc2cnc(-n3c(C(F)(F)F)nc4c(N5CC[C@H](N(C)C(C)C)C5)nc(-c5onc6c5CCC[C@@]65CCCCC5=O)nc43)cc2n1C] | 0.99 | 677 | A | B |
| T-1i | 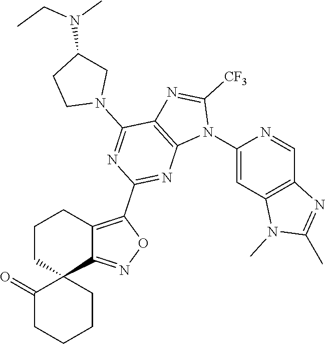 [SMILES: CCN(C)[C@H]1CCN(c2nc(-c3onc4c3CCC[C@@]43CCCCC3=O)nc3c2nc(C(F)(F)F)n3-c2cc3c(cn2)nc(C)n3C)C1] | 1.07 | 663 | A | B |
| T-1j | 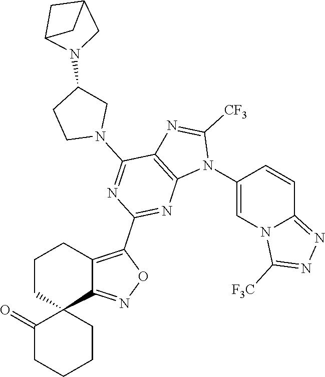 [SMILES: O=C1CCCC[C@@]12CCCc1c2noc1-c1nc(N2CC[C@H](N3CC4CC3C4)C2)c2nc(C(F)(F)F)n(-c3ccc4nnc(C(F)(F)F)n4c3)c2n1] | 1.19 | 726 | A | A |
| T-1k | 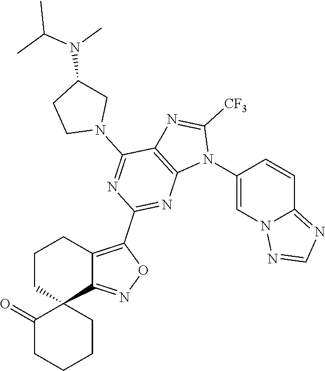 [SMILES: CC(C)N(C)[C@H]1CCN(c2nc(-c3onc4c3CCC[C@@]43CCCCC3=O)nc3c2nc(C(F)(F)F)n3-c2ccc3ncnn3c2)C1] | 1.11 | 649 | A | A |
| T-1l | 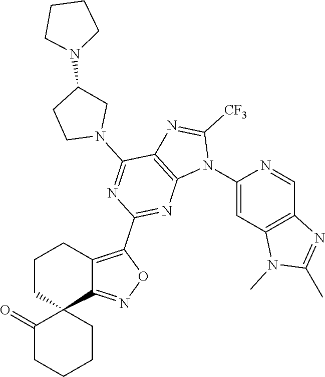 [SMILES: Cc1nc2cnc(-n3c(C(F)(F)F)nc4c(N5CC[C@H](N6CCCC6)C5)nc(-c5onc6c5CCC[C@@]65CCCCC5=O)nc43)cc2n1C] | 1.08 | 675 | A | B |
| T-1m | 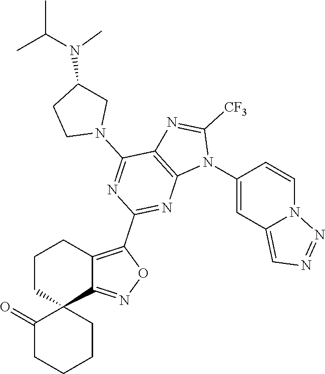 [SMILES: CC(C)N(C)[C@H]1CCN(c2nc(-c3onc4c3CCC[C@@]43CCCCC3=O)nc3c2nc(C(F)(F)F)n3-c2ccn3nncc3c2)C1] | 1.11 | 649 | A | A |
| T-1n | 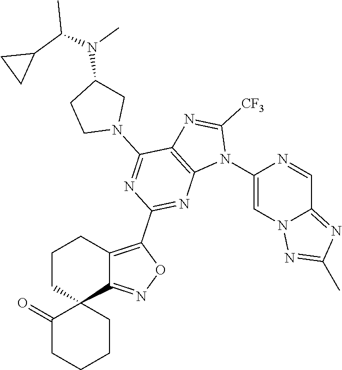 [SMILES: Cc1nc2cnc(-n3c(C(F)(F)F)nc4c(N5CC[C@H](N(C)[C@@H](C)C6CC6)C5)nc(-c5onc6c5CCC[C@@]65CCCCC5=O)nc43)cn2n1] | 1.15 | 690 | A | B |
| T-1o | 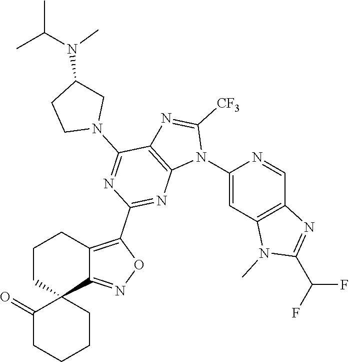 [SMILES: CC(C)N(C)[C@H]1CCN(c2nc(-c3onc4c3CCC[C@@]43CCCCC3=O)nc3c2nc(C(F)(F)F)n3-c2cc3c(cn2)nc(C(F)F)n3C)C1] | 1.15 | 713 | A | B |
| T-1p | 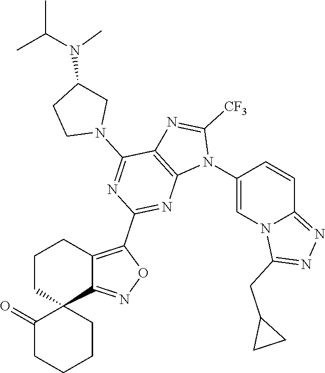 [SMILES: CC(C)N(C)[C@H]1CCN(c2nc(-c3onc4c3CCC[C@@]43CCCCC3=O)nc3c2nc(C(F)(F)F)n3-c2ccc3nnc(CC4CC4)n3c2)C1] | 1.09 | 702 | A | A |
| T-1q | 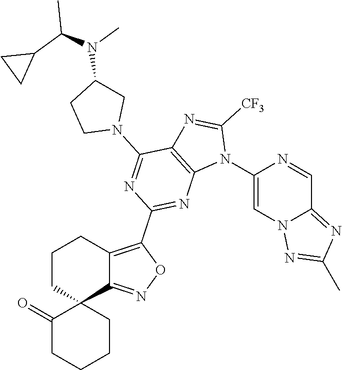 [SMILES: Cc1nc2cnc(-n3c(C(F)(F)F)nc4c(N5CC[C@H](N(C)[C@H](C)C6CC6)C5)nc(-c5onc6c5CCC[C@@]65CCCCC5=O)nc43)cn2n1] | 0.89 | 708 | A | B |
| T-1r | 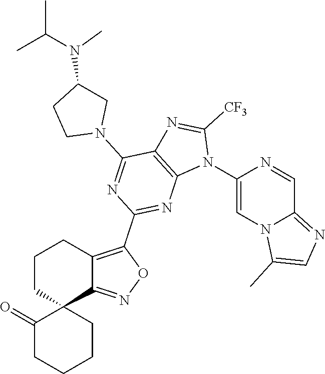 [SMILES: Cc1cnc2cnc(-n3c(C(F)(F)F)nc4c(N5CC[C@H](N(C)C(C)C)C5)nc(-c5onc6c5CCC[C@@]65CCCCC5=O)nc43)cn12] | 1.11 | 663 | A | A |
| T-1s | 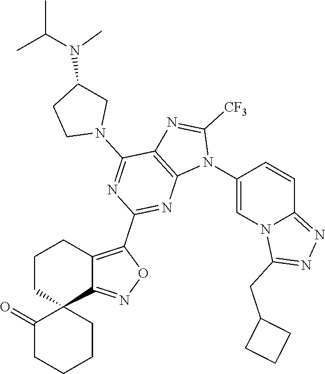 [SMILES: CC(C)N(C)[C@H]1CCN(c2nc(-c3onc4c3CCC[C@@]43CCCCC3=O)nc3c2nc(C(F)(F)F)n3-c2ccc3nnc(CC4CCC4)n3c2)C1] | 1.11 | 717 | A | A |
| T-1t | 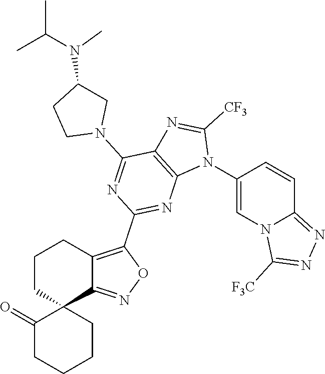 [SMILES: CC(C)N(C)[C@H]1CCN(c2nc(-c3onc4c3CCC[C@@]43CCCCC3=O)nc3c2nc(C(F)(F)F)n3-c2ccc3nnc(C(F)(F)F)n3c2)C1] | 1.13 | 717 | A | A |
| T-1u | 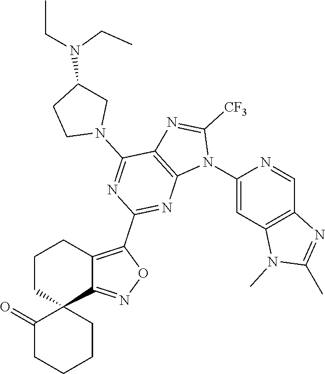 [SMILES: CCN(CC)[C@H]1CCN(c2nc(-c3onc4c3CCC[C@@]43CCCCC3=O)nc3c2nc(C(F)(F)F)n3-c2cc3c(cn2)nc(C)n3C)C1] | 1.11 | 677 | A | B |
| T-1v | 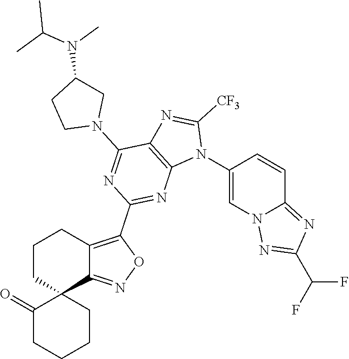 [SMILES: CC(C)N(C)[C@H]1CCN(c2nc(-c3onc4c3CCC[C@@]43CCCCC3=O)nc3c2nc(C(F)(F)F)n3-c2ccc3nc(C(F)F)nn3c2)C1] | 1.05 | 699 | A | A |
| T-1w | 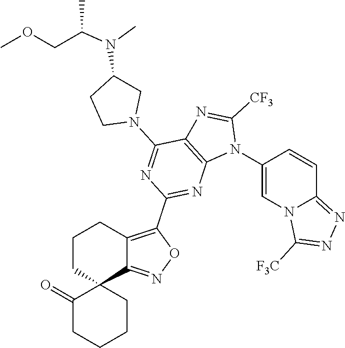 [SMILES: COC[C@H](C)N(C)[C@H]1CCN(c2nc(-c3onc4c3CCC[C@@]43CCCCC3=O)nc3c2nc(C(F)(F)F)n3-c2ccc3nnc(C(F)(F)F)n3c2)C1] | 1.13 | 747 | A | A |
| T-1x | 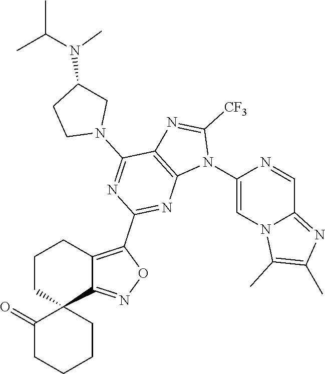 [SMILES: Cc1nc2cnc(-n3c(C(F)(F)F)nc4c(N5CC[C@H](N(C)C(C)C)C5)nc(-c5onc6c5CCC[C@@]65CCCCC5=O)nc43)cn2c1C] | 1.14 | 677 | A | B |
| T-1y | 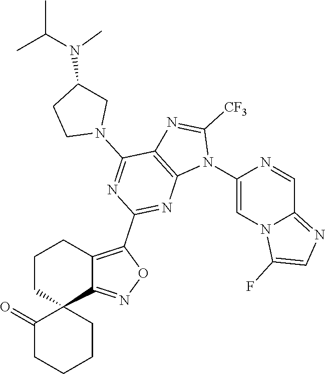 [SMILES: CC(C)N(C)[C@H]1CCN(c2nc(-c3onc4c3CCC[C@@]43CCCCC3=O)nc3c2nc(C(F)(F)F)n3-c2cn3c(F)cnc3cn2)C1] | 1.09 | 667 | A | A |
| T-1z | 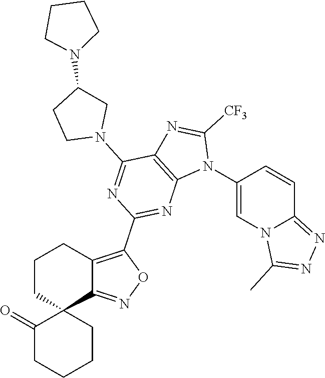 [SMILES: Cc1nnc2ccc(-n3c(C(F)(F)F)nc4c(N5CC[C@H](N6CCCC6)C5)nc(-c5onc6c5CCC[C@@]65CCCCC5=O)nc43)cn12] | 1.05 | 661 | A | A |
| T-1aa | 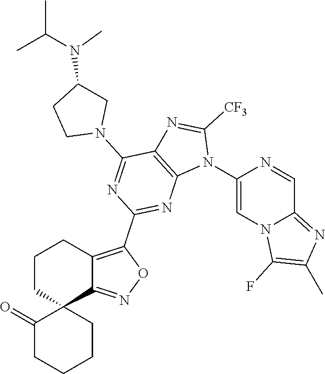 [SMILES: Cc1nc2cnc(-n3c(C(F)(F)F)nc4c(N5CC[C@H](N(C)C(C)C)C5)nc(-c5onc6c5CCC[C@@]65CCCCC5=O)nc43)cn2c1F] | 1.02 | 681 | A | B |
| T-1ab | 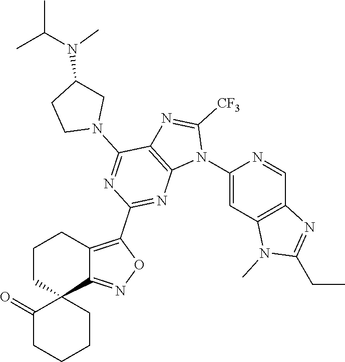 [SMILES: CCc1nc2cnc(-n3c(C(F)(F)F)nc4c(N5CC[C@H](N(C)C(C)C)C5)nc(-c5onc6c5CCC[C@@]65CCCCC5=O)nc43)cc2n1C] | 1.11 | 691 | A | B |
| T-1ac | 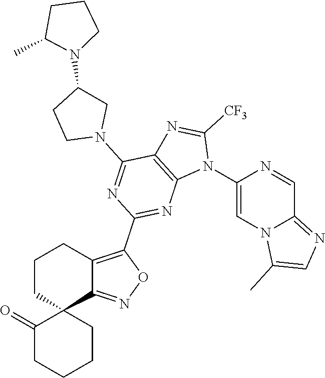 [SMILES: Cc1cnc2cnc(-n3c(C(F)(F)F)nc4c(N5CC[C@H](N6CCC[C@H]6C)C5)nc(-c5onc6c5CCC[C@@]65CCCCC5=O)nc43)cn12] | 1.14 | 675 | A | B |
| T-1ad | 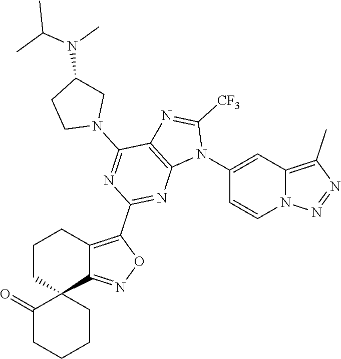 [SMILES: Cc1nnn2ccc(-n3c(C(F)(F)F)nc4c(N5CC[C@H](N(C)C(C)C)C5)nc(-c5onc6c5CCC[C@@]65CCCCC5=O)nc43)cc12] | 1.12 | 663 | A | A |
| T-1ae | 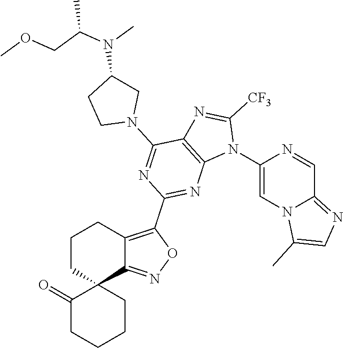 [SMILES: COC[C@H](C)N(C)[C@H]1CCN(c2nc(-c3onc4c3CCC[C@@]43CCCCC3=O)nc3c2nc(C(F)(F)F)n3-c2cn3c(C)cnc3cn2)C1] | 1.15 | 693 | A | B |
| T-1af | 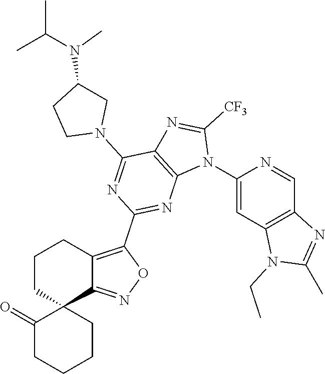 [SMILES: CCn1c(C)nc2cnc(-n3c(C(F)(F)F)nc4c(N5CC[C@H](N(C)C(C)C)C5)nc(-c5onc6c5CCC[C@@]65CCCCC5=O)nc43)cc21] | 1.12 | 691 | A | B |
| T-1ag | 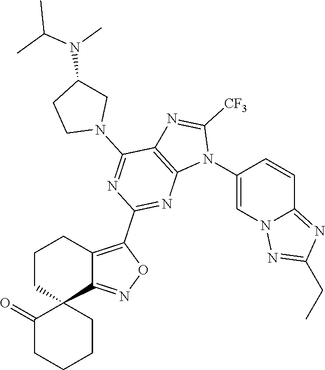 [SMILES: CCc1nc2ccc(-n3c(C(F)(F)F)nc4c(N5CC[C@H](N(C)C(C)C)C5)nc(-c5onc6c5CCC[C@@]65CCCCC5=O)nc43)cn2n1] | 1.18 | 677 | A | A |
| T-1ah | 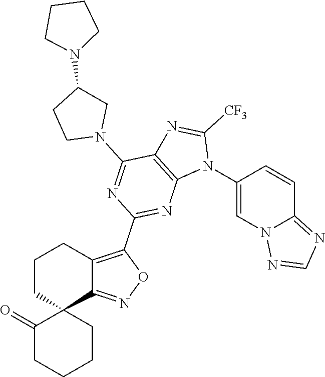 [SMILES: O=C1CCCC[C@@]12CCCc1c2noc1-c1nc(N2CC[C@H](N3CCCC3)C2)c2nc(C(F)(F)F)n(-c3ccc4ncnn4c3)c2n1] | 1.08 | 647 | A | A |
| T-1ai | 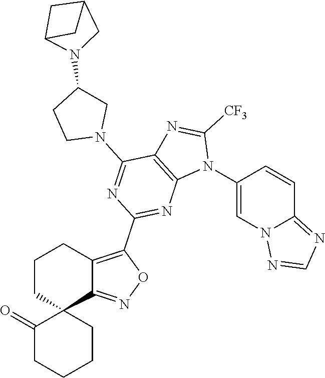 [SMILES: O=C1CCCC[C@@]12CCCc1c2noc1-c1nc(N2CC[C@H](N3CC4CC3C4)C2)c2nc(C(F)(F)F)n(-c3ccc4ncnn4c3)c2n1] | 1.13 | 659 | A | A |
| T-1aj | 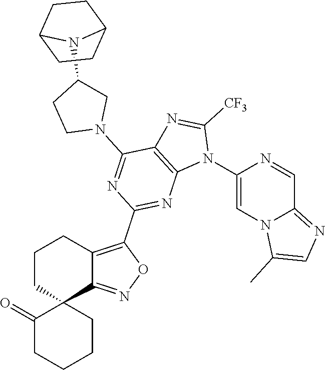 [SMILES: Cc1cnc2cnc(-n3c(C(F)(F)F)nc4c(N5CC[C@H](N6C7CCC6CC7)C5)nc(-c5onc6c5CCC[C@@]65CCCCC5=O)nc43)cn12] | 1.08 | 689 | A | B |
| T-1ak | 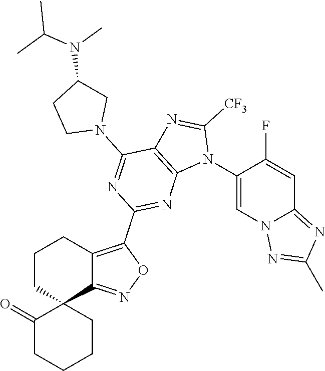 [SMILES: Cc1nc2cc(F)c(-n3c(C(F)(F)F)nc4c(N5CC[C@H](N(C)C(C)C)C5)nc(-c5onc6c5CCC[C@@]65CCCCC5=O)nc43)cn2n1] | 1.11 | 681 | A | A |
| T-1al | 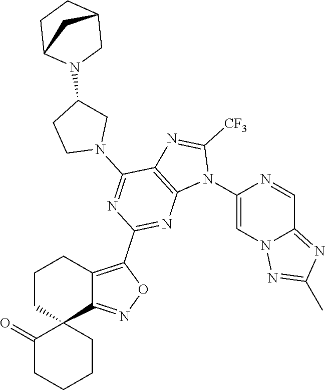 [SMILES: Cc1nc2cnc(-n3c(C(F)(F)F)nc4c(N5CC[C@H](N6C[C@H]7CC[C@@H]6C7)C5)nc(-c5onc6c5CCC[C@@]65CCCCC5=O)nc43)cn2n1] | 1.12 | 688 | A | B |
| T-1am | 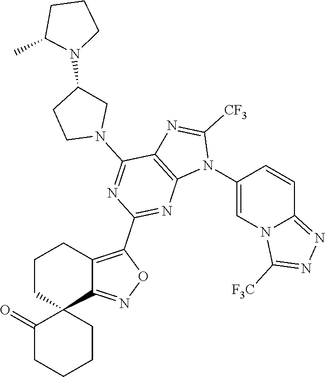 [SMILES: C[C@@H]1CCCN1[C@H]1CCN(c2nc(-c3onc4c3CCC[C@@]43CCCCC3=O)nc3c2nc(C(F)(F)F)n3-c2ccc3nnc(C(F)(F)F)n3c2)C1] | 1.11 | 729 | A | A |
| T-1an | 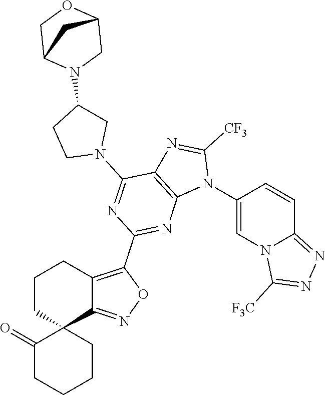 [SMILES: O=C1CCCC[C@@]12CCCc1c2noc1-c1nc(N2CC[C@H](N3C[C@@H]4C[C@H]3CO4)C2)c2nc(C(F)(F)F)n(-c3ccc4nnc(C(F)(F)F)n4c3)c2n1] | 1.13 | 743 | A | A |
| T-1ao | 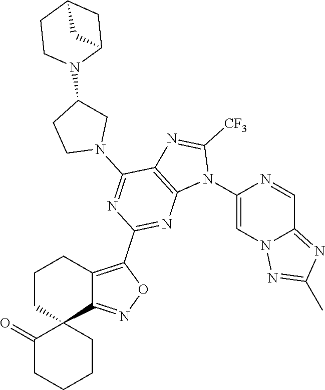 [SMILES: Cc1nc2cnc(-n3c(C(F)(F)F)nc4c(N5CC[C@H](N6CC[C@H]7C[C@@H]6C7)C5)nc(-c5onc6c5CCC[C@@]65CCCCC5=O)nc43)cn2n1] | 1.16 | 688 | A | B |
| T-1ap | 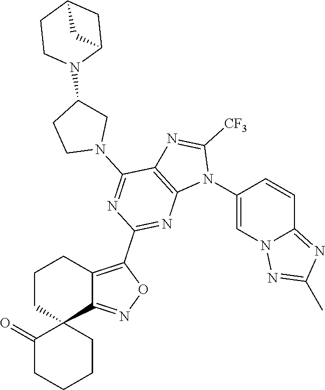 [SMILES: Cc1nc2ccc(-n3c(C(F)(F)F)nc4c(N5CC[C@H](N6CC[C@H]7C[C@@H]6C7)C5)nc(-c5onc6c5CCC[C@@]65CCCCC5=O)nc43)cn2n1] | 1.09 | 687 | A | A |
| T-1aq | 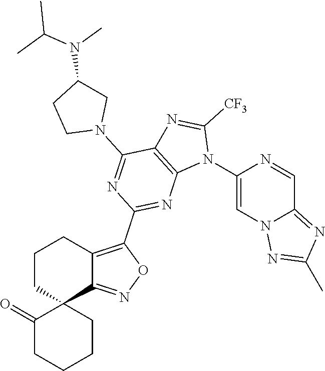 [SMILES: Cc1nc2cnc(-n3c(C(F)(F)F)nc4c(N5CC[C@H](N(C)C(C)C)C5)nc(-c5onc6c5CCC[C@@]65CCCCC5=O)nc43)cn2n1] | 0.93 | 682 | A | A |
| T-1ar | 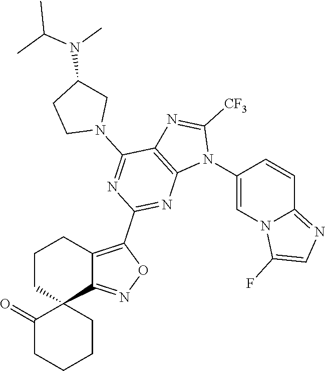 [SMILES: CC(C)N(C)[C@H]1CCN(c2nc(-c3onc4c3CCC[C@@]43CCCCC3=O)nc3c2nc(C(F)(F)F)n3-c2ccc3ncc(F)n3c2)C1] | 1.11 | 666 | A | A |
| T-1as | 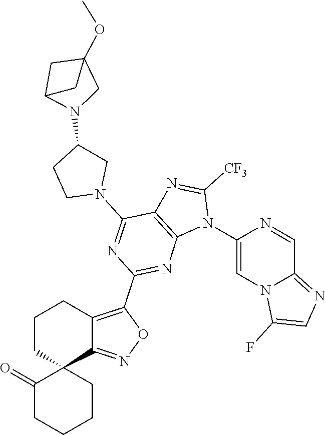 [SMILES: COC12CC(C1)N([C@H]1CCN(c3nc(-c4onc5c4CCC[C@@]54CCCCC4=O)nc4c3nc(C(F)(F)F)n4-c3cn4c(F)cnc4cn3)C1)C2] | 1.09 | 707 | A | B |
| T-1at | 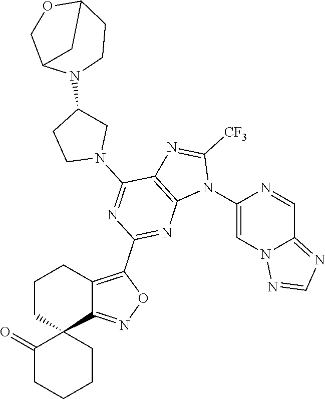 [SMILES: O=C1CCCC[C@@]12CCCc1c2noc1-c1nc(N2CC[C@H](N3CCC4CC3CO4)C2)c2nc(C(F)(F)F)n(-c3cn4ncnc4cn3)c2n1] | 1.09 | 690 | A | B |
| T-1au | 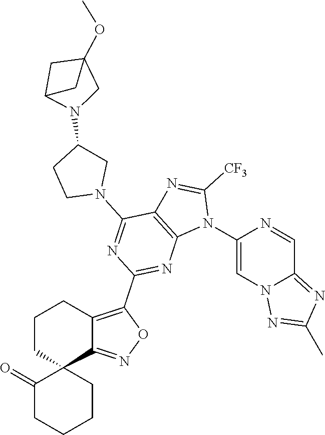 [SMILES: COC12CC(C1)N([C@H]1CCN(c3nc(-c4onc5c4CCC[C@@]54CCCCC4=O)nc4c3nc(C(F)(F)F)n4-c3cn4nc(C)nc4cn3)C1)C2] | 1.12 | 703 | A | B |
| T-1av | 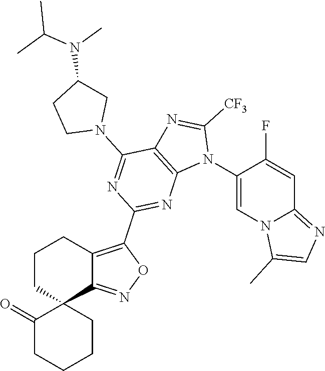 [SMILES: Cc1cnc2cc(F)c(-n3c(C(F)(F)F)nc4c(N5CC[C@H](N(C)C(C)C)C5)nc(-c5onc6c5CCC[C@@]65CCCCC5=O)nc43)cn12] | 1.10 | 680 | A | A |
| T-1aw | 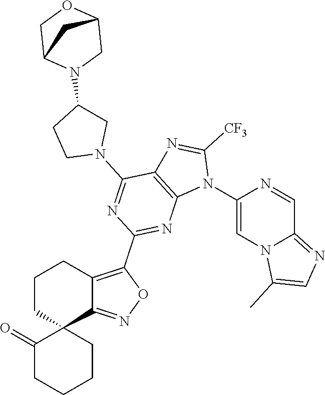 [SMILES: Cc1cnc2cnc(-n3c(C(F)(F)F)nc4c(N5CC[C@H](N6C[C@@H]7C[C@H]6CO7)C5)nc(-c5onc6c5CCC[C@@]65CCCCC5=O)nc43)cn12] | 1.14 | 689 | A | A |
| T-1ax | 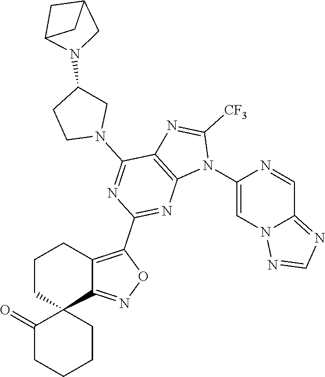 [SMILES: O=C1CCCC[C@@]12CCCc1c2noc1-c1nc(N2CC[C@H](N3CC4CC3C4)C2)c2nc(C(F)(F)F)n(-c3cn4ncnc4cn3)c2n1] | 1.08 | 660 | A | B |
| T-1ay | 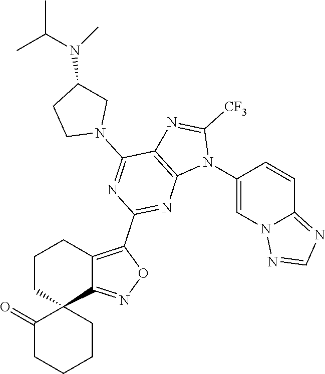 [SMILES: CC(C)N(C)[C@H]1CCN(c2nc(-c3onc4c3CCC[C@@]43CCCCC3=O)nc3c2nc(C(F)(F)F)n3-c2ccc3ncnn3c2)C1] | 1.11 | 650 | A | A |
| T-1az | 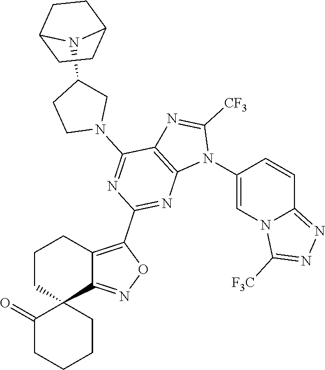 [SMILES: O=C1CCCC[C@@]12CCCc1c2noc1-c1nc(N2CC[C@H](N3C4CCC3CC4)C2)c2nc(C(F)(F)F)n(-c3ccc4nnc(C(F)(F)F)n4c3)c2n1] | 1.15 | 741 | A | A |
| T-1ba | 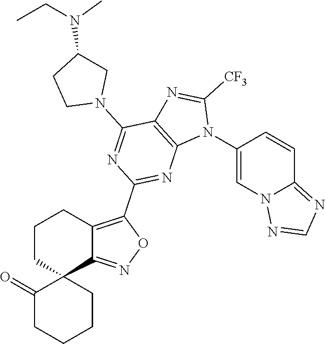 [SMILES: CCN(C)[C@H]1CCN(c2nc(-c3onc4c3CCC[C@@]43CCCCC3=O)nc3c2nc(C(F)(F)F)n3-c2ccc3ncnn3c2)C1] | 1.07 | 635 | A | A |
| T-1bb | 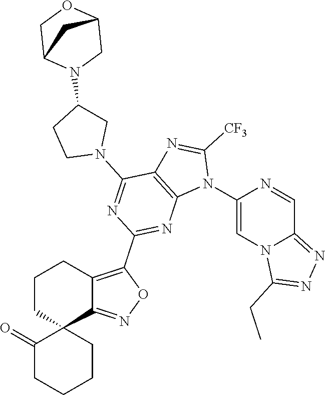 [SMILES: CCc1nnc2cnc(-n3c(C(F)(F)F)nc4c(N5CC[C@H](N6C[C@@H]7C[C@H]6CO7)C5)nc(-c5onc6c5CCC[C@@]65CCCCC5=O)nc43)cn12] | 1.12 | 703 | A | B |
| T-1bc | 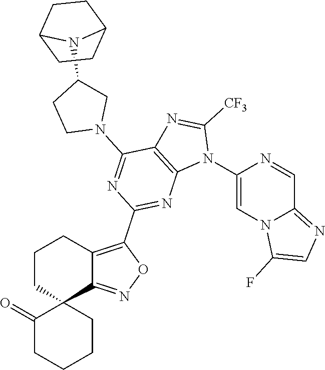 [SMILES: O=C1CCCC[C@@]12CCCc1c2noc1-c1nc(N2CC[C@H](N3C4CCC3CC4)C2)c2nc(C(F)(F)F)n(-c3cn4c(F)cnc4cn3)c2n1] | 1.13 | 691 | A | A |
| T-1bd | 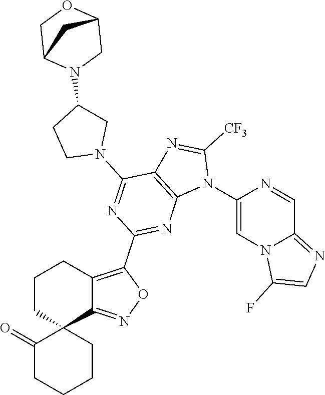 [SMILES: O=C1CCCC[C@@]12CCCc1c2noc1-c1nc(N2CC[C@H](N3C[C@@H]4C[C@H]3CO4)C2)c2nc(C(F)(F)F)n(-c3cn4c(F)cnc4cn3)c2n1] | 1.12 | 693 | A | B |
| T-1be | 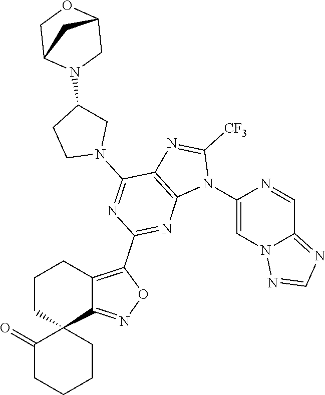 [SMILES: O=C1CCCC[C@@]12CCCc1c2noc1-c1nc(N2CC[C@H](N3C[C@@H]4C[C@H]3CO4)C2)c2nc(C(F)(F)F)n(-c3cn4ncnc4cn3)c2n1] | 1.09 | 676 | A | B |
| T-1bf | 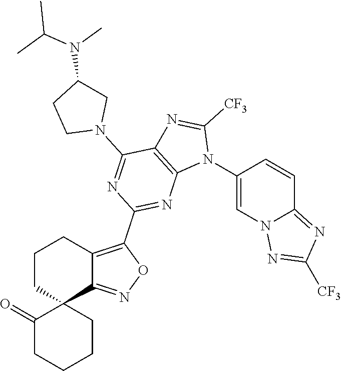 [SMILES: CC(C)N(C)[C@H]1CCN(c2nc(-c3onc4c3CCC[C@@]43CCCCC3=O)nc3c2nc(C(F)(F)F)n3-c2ccc3nc(C(F)(F)F)nn3c2)C1] | 1.11 | 717 | A | A |
| T-1bg | 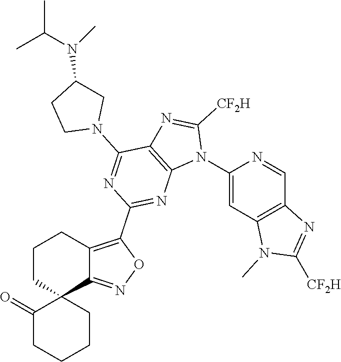 [SMILES: CC(C)N(C)[C@H]1CCN(c2nc(-c3onc4c3CCC[C@@]43CCCCC3=O)nc3c2nc(C(F)F)n3-c2cc3c(cn2)nc(C(F)F)n3C)C1] | 1.13 | 695 | A | B |
| T-1bh | 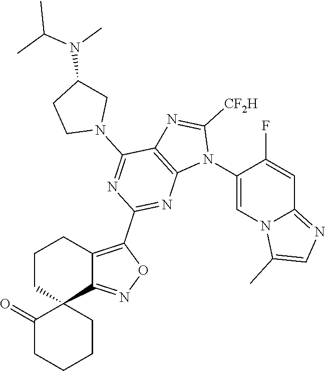 [SMILES: Cc1cnc2cc(F)c(-n3c(C(F)F)nc4c(N5CC[C@H](N(C)C(C)C)C5)nc(-c5onc6c5CCC[C@@]65CCCCC5=O)nc43)cn12] | 1.05 | 662 | A | A |
| T-1bi | 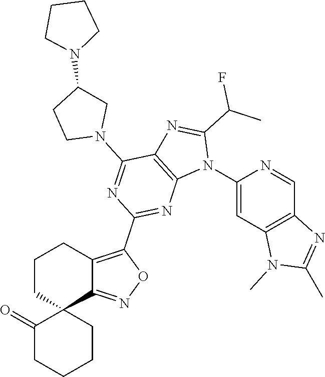 [SMILES: Cc1nc2cnc(-n3c(C(C)F)nc4c(N5CC[C@H](N6CCCC6)C5)nc(-c5onc6c5CCC[C@@]65CCCCC5=O)nc43)cc2n1C] | 1.07 | 653 | A | B |
| T-1bj | 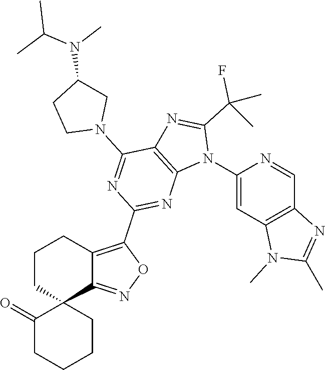 [SMILES: Cc1nc2cnc(-n3c(C(C)(C)F)nc4c(N5CC[C@H](N(C)C(C)C)C5)nc(-c5onc6c5CCC[C@@]65CCCCC5=O)nc43)cc2n1C] | 1.03 | 669 | A | B |
| T-1bk | 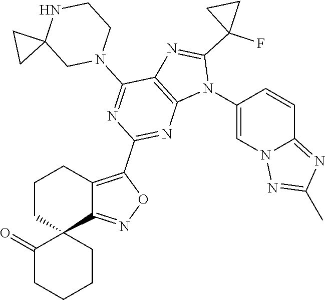 [SMILES: Cc1nc2ccc(-n3c(C4(F)CC4)nc4c(N5CCNC6(CC6)C5)nc(-c5onc6c5CCC[C@@]65CCCCC5=O)nc43)cn2n1] | 0.95 | 623 | A | A |
| T-1bl | 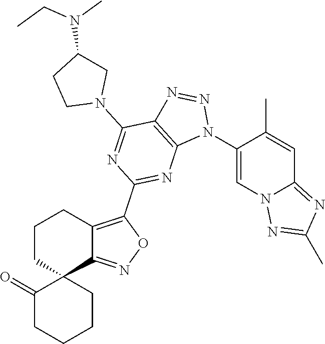 [SMILES: CCN(C)[C@H]1CCN(c2nc(-c3onc4c3CCC[C@@]43CCCCC3=O)nc3c2nnn3-c2cn3nc(C)nc3cc2C)C1] | 1.05 | 619 | A | A |
| T-1bm | 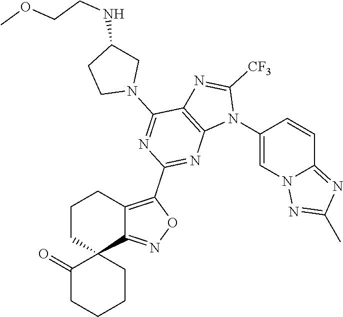 | 1.03 | 665 | A | A |
| T-1bn | 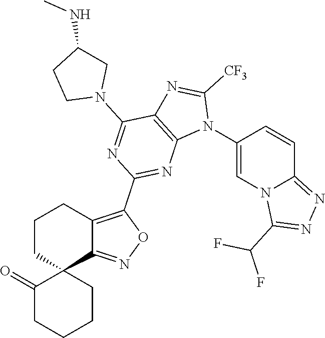 [SMILES: CN[C@H]1CCN(c2nc(-c3onc4c3CCC[C@@]43CCCCC3=O)nc3c2nc(C(F)(F)F)n3-c2ccc3nnc(C(F)F)n3c2)C1] | 1.08 | 657 | A | A |
| T-1bp | 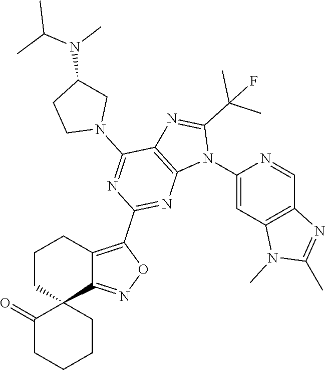 [SMILES: Cc1nc2cnc(-n3c(C(C)(C)F)nc4c(N5CC[C@H](N(C)C(C)C)C5)nc(-c5onc6c5CCC[C@@]65CCCCC5=O)nc43)cc2n1C] | 1.03 | 669 | A | B |
| T-1bq | 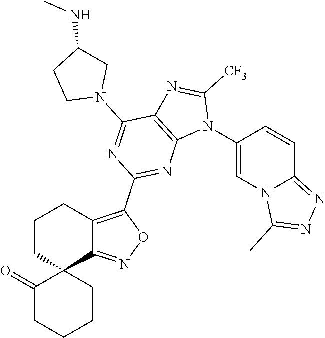 [SMILES: CN[C@H]1CCN(c2nc(-c3onc4c3CCC[C@@]43CCCCC3=O)nc3c2nc(C(F)(F)F)n3-c2ccc3nnc(C)n3c2)C1] | 0.90 | 621 | C | A |
| T-1bs | 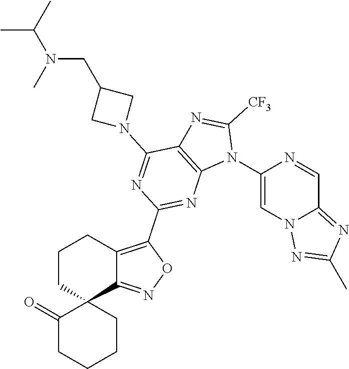 [SMILES: Cc1nc2cnc(-n3c(C(F)(F)F)nc4c(N5CC(CN(C)C(C)C)C5)nc(-c5onc6c5CCC[C@@]65CCCCC5=O)nc43)cn2n1] | 0.87 | 681 | C | B |
| T-1bt | 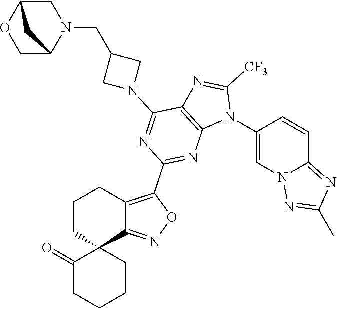 [SMILES: Cc1nc2ccc(-n3c(C(F)(F)F)nc4c(N5CC(CN6C[C@@H]7C[C@H]6CO7)C5)nc(-c5onc6c5CCC[C@@]65CCCCC5=O)nc43)cn2n1] | 0.77 | 770 | C | B |
| T-1bu | 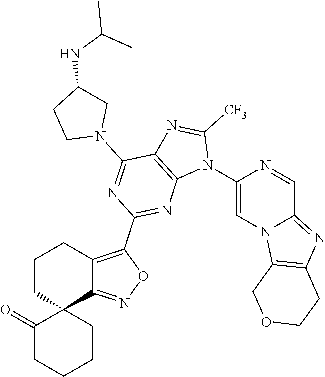 [SMILES: CC(C)N[C@H]1CCN(c2nc(-c3onc4c3CCC[C@@]43CCCCC3=O)nc3c2nc(C(F)(F)F)n3-c2cn3c4c(nc3cn2)CCOC4)C1] | 1.03 | 691 | C | B |
| T-1bv | 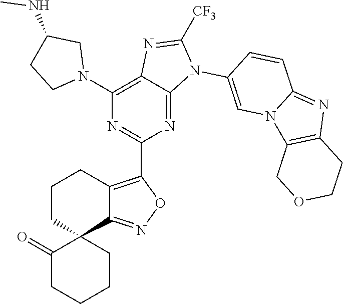 [SMILES: CN[C@H]1CCN(c2nc(-c3onc4c3CCC[C@@]43CCCCC3=O)nc3c2nc(C(F)(F)F)n3-c2ccc3nc4c(n3c2)COCC4)C1] | 0.94 | 662 | C | A |
| T-1bw | 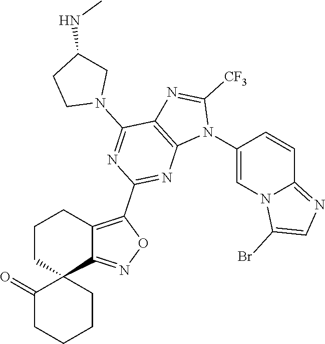 [SMILES: CN[C@H]1CCN(c2nc(-c3onc4c3CCC[C@@]43CCCCC3=O)nc3c2nc(C(F)(F)F)n3-c2ccc3ncc(Br)n3c2)C1] | 1.02 | 684 | C | A |
| T-1bx | 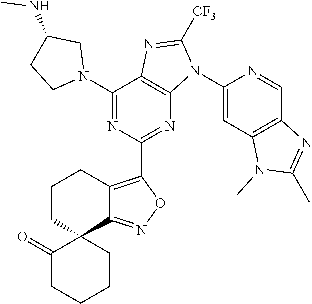 | 0.97 | 635 | C | B |
| T-1by | 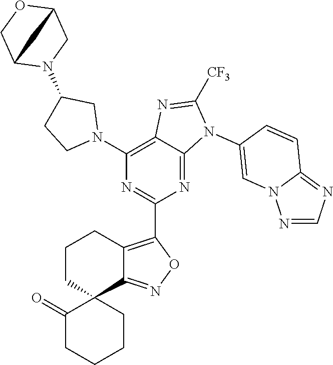 [SMILES: O=C1CCCC[C@@]12CCCc1c2noc1-c1nc(N2CC[C@H](N3C[C@@H]4C[C@H]3CO4)C2)c2nc(C(F)(F)F)n(-c3ccc4ncnn4c3)c2n1] | 0.95 | 675 | C | A |
| T-1bz | 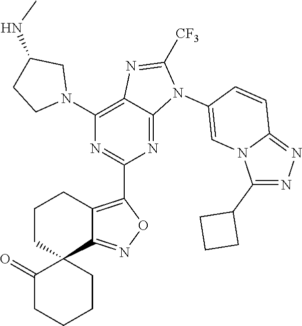 [SMILES: CN[C@H]1CCN(c2nc(-c3onc4c3CCC[C@@]43CCCCC3=O)nc3c2nc(C(F)(F)F)n3-c2ccc3nnc(C4CCC4)n3c2)C1] | 0.98 | 661 | C | A |
| T-1ca | 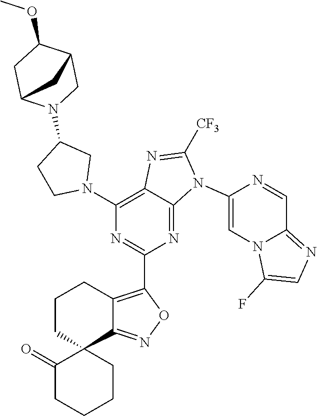 [SMILES: CO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3c(F)cnc3cn2)C1] | 1.05 | 721 | C | B |
| T-1cb | 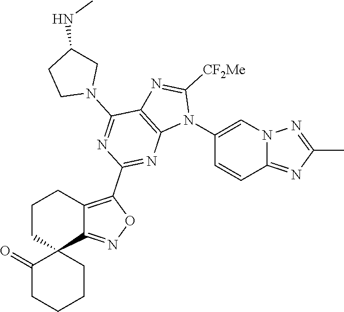 | 1.21 | 616 | A | A |
| T-1cc | 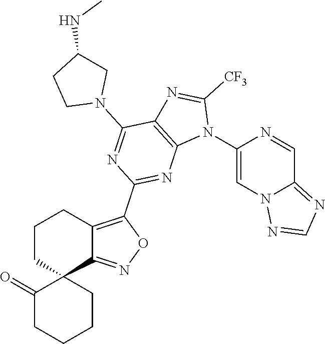 [SMILES: CN[C@H]1CCN(c2nc(-c3onc4c3CCC[C@@]43CCCCC3=O)nc3c2nc(C(F)(F)F)n3-c2cn3ncnc3cn2)C1] | 1.14 | 682 | A | A |
| T-1cd | 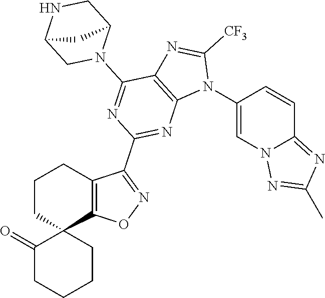 [SMILES: Cc1nc2ccc(-n3c(C(F)(F)F)nc4c(N5C[C@@H]6C[C@H]5CN6)nc(-c5noc6c5CCC[C@@]65CCCCC5=O)nc43)cn2n1] | 1.19 | 618 | A | A |
| T-1ce | 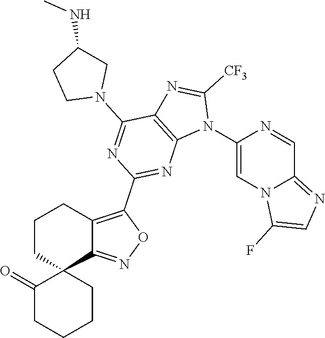 [SMILES: CN[C@H]1CCN(c2nc(-c3onc4c3CCC[C@@]43CCCCC3=O)nc3c2nc(C(F)(F)F)n3-c2cn3c(F)cnc3cn2)C1] | 1.20 | 625 | A | B |
| T-1cf | 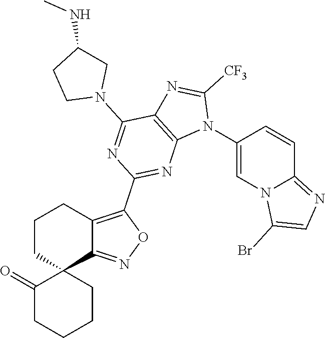 [SMILES: CN[C@H]1CCN(c2nc(-c3onc4c3CCC[C@@]43CCCCC3=O)nc3c2nc(C(F)(F)F)n3-c2ccc3ncc(Br)n3c2)C1] | 1.24 | 733 | A | A |
| T-1cg | 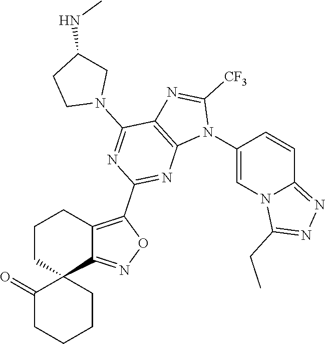 [SMILES: CCc1nnc2ccc(-n3c(C(F)(F)F)nc4c(N5CC[C@H](NC)C5)nc(-c5onc6c5CCC[C@@]65CCCCC5=O)nc43)cn12] | 1.26 | 635 | A | A |
| T-1ch | 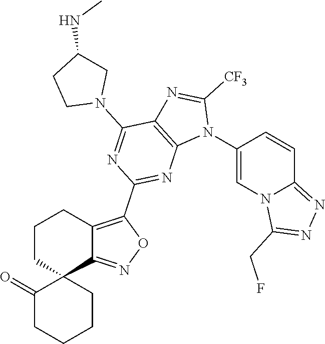 [SMILES: CN[C@H]1CCN(c2nc(-c3onc4c3CCC[C@@]43CCCCC3=O)nc3c2nc(C(F)(F)F)n3-c2ccc3nnc(CF)n3c2)C1] | 1.21 | 638 | A | A |
| T-1ci | 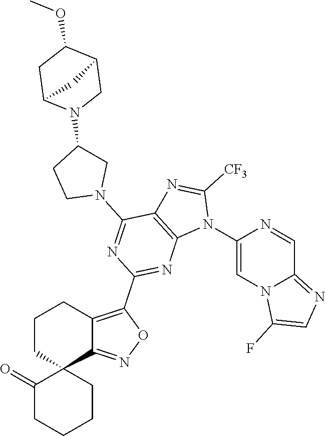 [SMILES: CO[C@H]1C[C@H]2C[C@@H]1CN2[C@H]1CCN(c2nc(-c3onc4c3CCC[C@@]43CCCCC3=O)nc3c2nc(C(F)(F)F)n3-c2cn3c(F)cnc3cn2)C1] | 0.91 | 717 | A | A |
| T-1cj | 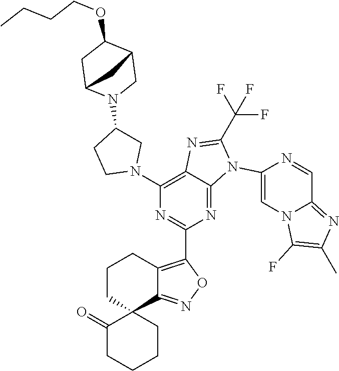 [SMILES: CCCCO[C@@H]1C[C@@H]2C[C@H]1CN2[C@H]1CCN(c2nc(-c3onc4c3CCC[C@@]43CCCCC3=O)nc3c2nc(C(F)(F)F)n3-c2cn3c(F)c(C)nc3cn2)C1] | 0.97 | 797 | A | A |
| T-1ck | 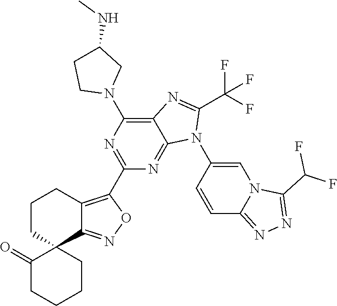 [SMILES: CN[C@H]1CCN(c2nc(-c3onc4c3CCC[C@@]43CCCCC3=O)nc3c2nc(C(F)(F)F)n3-c2ccc3nnc(C(F)F)n3c2)C1] | 1.15 | 656 | A | A |

### Reductive Amination

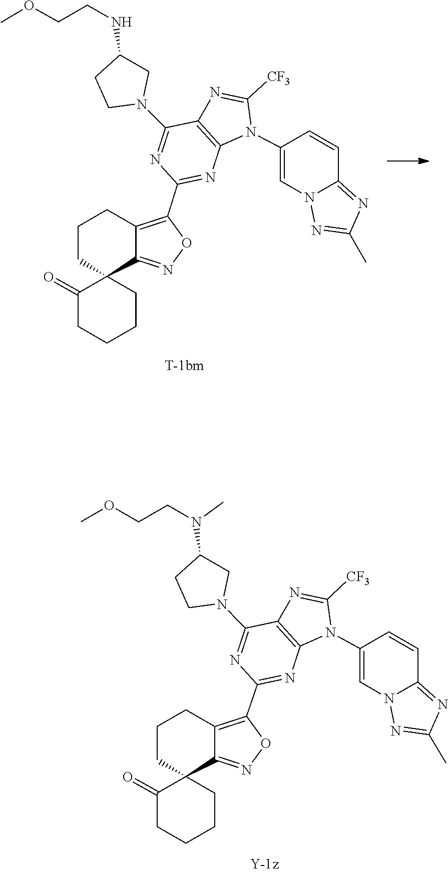

A solution of T-1bm (73 mg, 0.11 mmQ-1aol, 1.0 equiv.) in methanol is stirred with formaldehyde (9 mg, 0.121 mmol, 1.1 equiv.) and a drop of acetic acid for 5 minutes. Sodium triacetoxy borohydride (52 mg, 0.24 mmol, 2.2 equiv.) is added and the reaction is allowed to proceed for 30 minutes. The reaction mixture is diluted with water and then extracted with CH₂Cl₂ three times. The combined extracts were dried with Na₂SO₄ and evaporated to dryness. The crude product was purified by RP-chromatography to give the desired product Y-1z (HPLC method A, tᵣₑₜ=1.03 min; [M+H]=679).

The following intermediate compounds Y-1 (Table 22) are obtained analogously using the indicated carbonyl component.

TABLE 22

| | | tᵣₑₜ | | HPLC | Carbonyl |
|---|---|---|---|---|---|
| # | Structure | [min] | [M + H]⁺ | method | component |
| Y-1a | 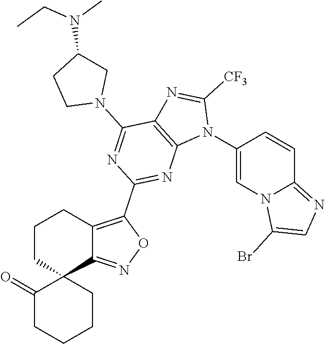 [SMILES: CCN(C)[C@H]1CCN(c2nc(-c3onc4c3CCC[C@@]43CCCCC3=O)nc3c2nc(C(F)(F)F)n3-c2ccc3ncc(Br)n3c2)C1] | 1.12 | 713 | A | 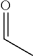 |
| Y-1b | 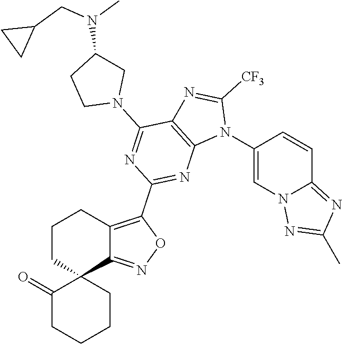 [SMILES: Cc1nc2ccc(-n3c(C(F)(F)F)nc4c(N5CC[C@H](N(C)CC6CC6)C5)nc(-c5onc6c5CCC[C@@]65CCCCC5=O)nc43)cn2n1] | 1.17 | 675 | A | 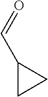 |
| Y-1c | 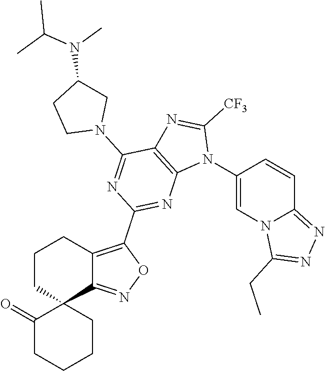 [SMILES: CCc1nnc2ccc(-n3c(C(F)(F)F)nc4c(N5CC[C@H](N(C)C(C)C)C5)nc(-c5onc6c5CCC[C@@]65CCCCC5=O)nc43)cn12] | 1.02 | 677 | A | 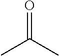 |
| Y-1d | 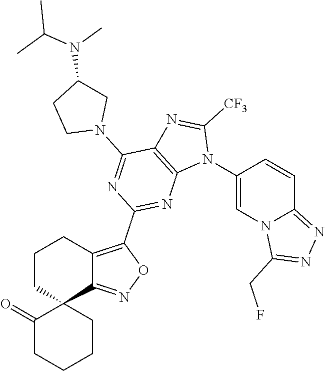 [SMILES: CC(C)N(C)[C@H]1CCN(c2nc(-c3onc4c3CCC[C@@]43CCCCC3=O)nc3c2nc(C(F)(F)F)n3-c2ccc3nnc(CF)n3c2)C1] | 1.06 | 681 | A | 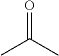 |
| Y-1e | 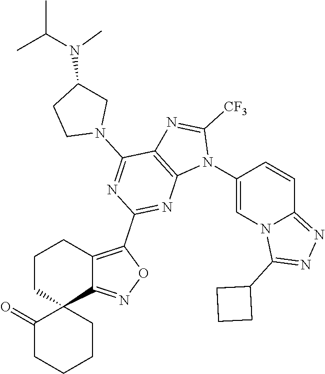 [SMILES: CC(C)N(C)[C@H]1CCN(c2nc(-c3onc4c3CCC[C@@]43CCCCC3=O)nc3c2nc(C(F)(F)F)n3-c2ccc3nnc(C4CCC4)n3c2)C1] | 1.11 | 703 | A | 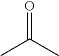 |
| Y-1f | 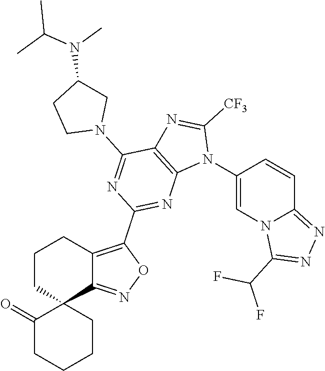 [SMILES: CC(C)N(C)[C@H]1CCN(c2nc(-c3onc4c3CCC[C@@]43CCCCC3=O)nc3c2nc(C(F)(F)F)n3-c2ccc3nnc(C(F)F)n3c2)C1] | 1.02 | 699 | A | 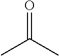 |
| Y-1g | 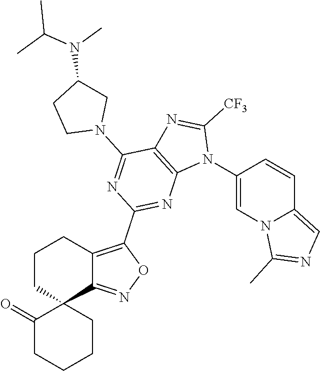 [SMILES: Cc1ncc2ccc(-n3c(C(F)(F)F)nc4c(N5CC[C@H](N(C)C(C)C)C5)nc(-c5onc6c5CCC[C@@]65CCCCC5=O)nc43)cn12] | 1.09 | 662 | A | 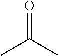 |
| Y-1h | 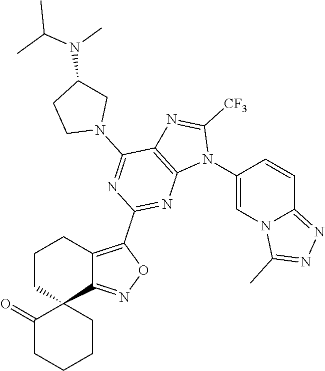 [SMILES: Cc1nnc2ccc(-n3c(C(F)(F)F)nc4c(N5CC[C@H](N(C)C(C)C)C5)nc(-c5onc6c5CCC[C@@]65CCCCC5=O)nc43)cn12] | 1.10 | 663 | A | 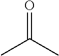 |
| Y-1i | 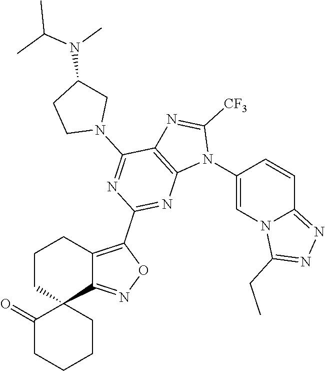 [SMILES: CCc1nnc2ccc(-n3c(C(F)(F)F)nc4c(N5CC[C@H](N(C)C(C)C)C5)nc(-c5onc6c5CCC[C@@]65CCCCC5=O)nc43)cn12] | 1.09 | 677 | A | 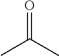 |
| Y-1j | 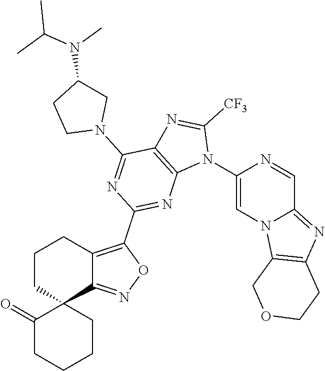 [SMILES: CC(C)N(C)[C@H]1CCN(c2nc(-c3onc4c3CCC[C@@]43CCCCC3=O)nc3c2nc(C(F)(F)F)n3-c2cn3c4c(nc3cn2)CCOC4)C1] | 1.13 | 705 | A | 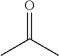 |
| Y-1k | 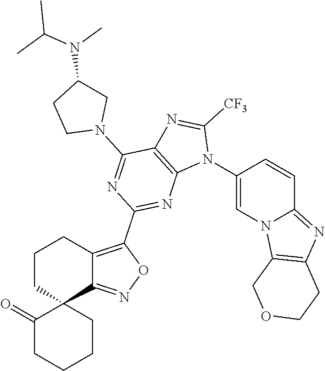 [SMILES: CC(C)N(C)[C@H]1CCN(c2nc(-c3onc4c3CCC[C@@]43CCCCC3=O)nc3c2nc(C(F)(F)F)n3-c2ccc3nc4c(n3c2)COCC4)C1] | 1.09 | 704 | A | 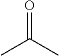 |
| Y-1l | 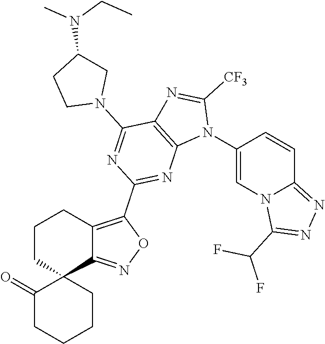 [SMILES: CCN(C)[C@H]1CCN(c2nc(-c3onc4c3CCC[C@@]43CCCCC3=O)nc3c2nc(C(F)(F)F)n3-c2ccc3nnc(C(F)F)n3c2)C1] | 1.11 | 685 | A | 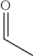 |
| Y-1m | 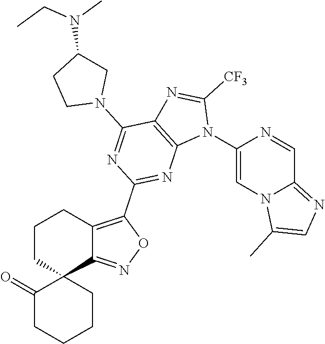 [SMILES: CCN(C)[C@H]1CCN(c2nc(-c3onc4c3CCC[C@@]43CCCCC3=O)nc3c2nc(C(F)(F)F)n3-c2cn3c(C)cnc3cn2)C1] | 1.08 | 649 | A | 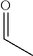 |
| Y-1n | 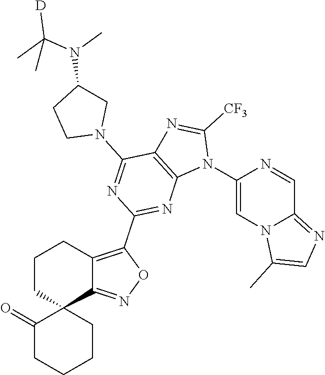 [SMILES: [2H]C(C)(C)N(C)[C@H]1CCN(c2nc(-c3onc4c3CCC[C@@]43CCCCC3=O)nc3c2nc(C(F)(F)F)n3-c2cn3c(C)cnc3cn2)C1] | 1.11 | 664 | A | 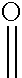 |
| Y-1o | 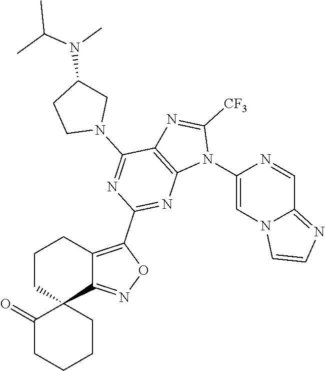 [SMILES: CC(C)N(C)[C@H]1CCN(c2nc(-c3onc4c3CCC[C@@]43CCCCC3=O)nc3c2nc(C(F)(F)F)n3-c2cn3ccnc3cn2)C1] | 1.04 | 649 | A | 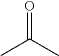 |
| Y-1p | 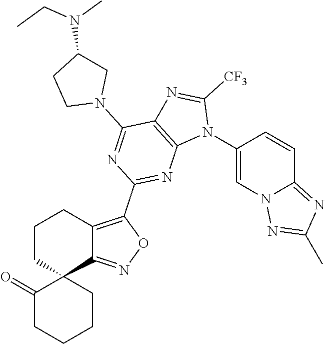 [SMILES: CCN(C)[C@H]1CCN(c2nc(-c3onc4c3CCC[C@@]43CCCCC3=O)nc3c2nc(C(F)(F)F)n3-c2ccc3nc(C)nn3c2)C1] | 1.09 | 649 | A | 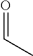 |
| Y-1q | 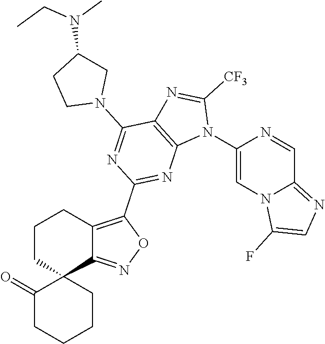 [SMILES: CCN(C)[C@H]1CCN(c2nc(-c3onc4c3CCC[C@@]43CCCCC3=O)nc3c2nc(C(F)(F)F)n3-c2cn3c(F)cnc3cn2)C1] | 1.12 | 653 | A | 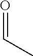 |
| Y-1r | 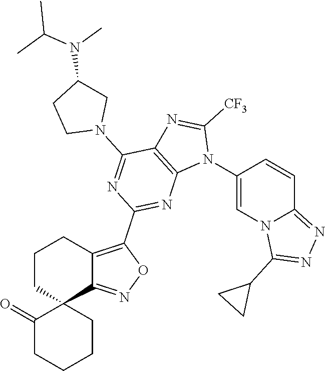 [SMILES: CC(C)N(C)[C@H]1CCN(c2nc(-c3onc4c3CCC[C@@]43CCCCC3=O)nc3c2nc(C(F)(F)F)n3-c2ccc3nnc(C4CC4)n3c2)C1] | 1.14 | 689 | A | 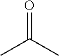 |
| Y-1s | 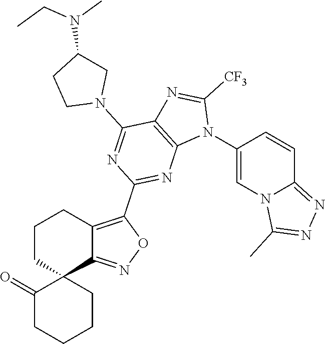 [SMILES: CCN(C)[C@H]1CCN(c2nc(-c3onc4c3CCC[C@@]43CCCCC3=O)nc3c2nc(C(F)(F)F)n3-c2ccc3nnc(C)n3c2)C1] | 1.09 | 649 | A | 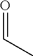 |
| Y-1t | 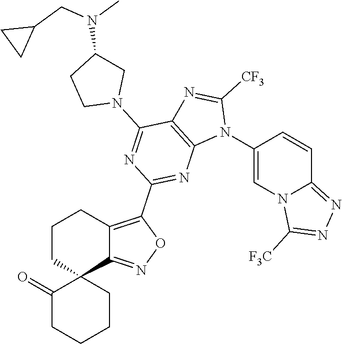 [SMILES: CN(CC1CC1)[C@H]1CCN(c2nc(-c3onc4c3CCC[C@@]43CCCCC3=O)nc3c2nc(C(F)(F)F)n3-c2ccc3nnc(C(F)(F)F)n3c2)C1] | 1.11 | 729 | A | 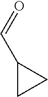 |
| Y-1u | 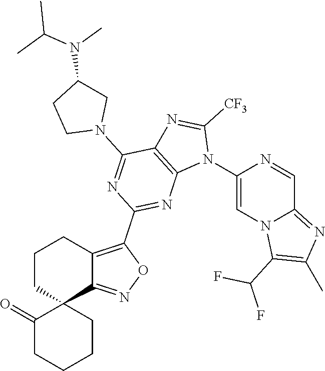 [SMILES: Cc1nc2cnc(-n3c(C(F)(F)F)nc4c(N5CC[C@H](N(C)C(C)C)C5)nc(-c5onc6c5CCC[C@@]65CCCCC5=O)nc43)cn2c1C(F)F] | 1.13 | 713 | A | 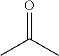 |
| Y-1v | 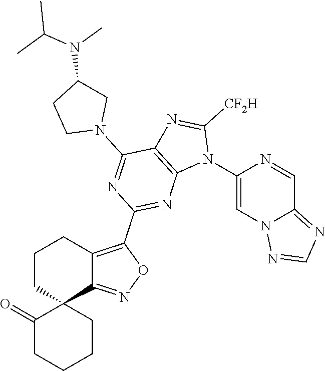 [SMILES: CC(C)N(C)[C@H]1CCN(c2nc(-c3onc4c3CCC[C@@]43CCCCC3=O)nc3c2nc(C(F)F)n3-c2cn3ncnc3cn2)C1] | 1.11 | 632 | A | 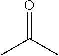 |
| Y-1w | 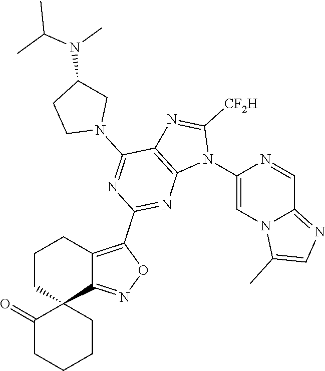 [SMILES: Cc1cnc2cnc(-n3c(C(F)F)nc4c(N5CC[C@H](N(C)C(C)C)C5)nc(-c5onc6c5CCC[C@@]65CCCCC5=O)nc43)cn12] | 1.14 | 645 | A | 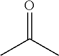 |
| Y-1x | 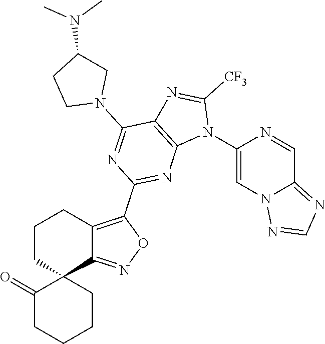 [SMILES: CN(C)[C@H]1CCN(c2nc(-c3onc4c3CCC[C@@]43CCCCC3=O)nc3c2nc(C(F)(F)F)n3-c2cn3ncnc3cn2)C1] | 1.07 | 622 | A | 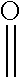 |
| Y-1y | 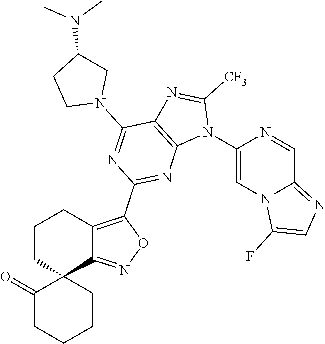 [SMILES: CN(C)[C@H]1CCN(c2nc(-c3onc4c3CCC[C@@]43CCCCC3=O)nc3c2nc(C(F)(F)F)n3-c2cn3c(F)cnc3cn2)C1] | 1.08 | 639 | A | 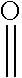 |
| Y-1z | 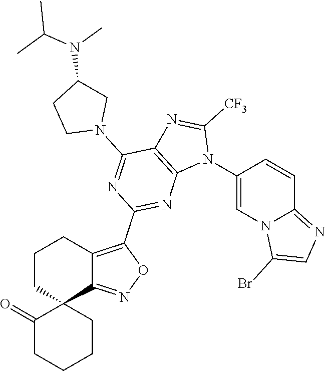 [SMILES: CC(C)N(C)[C@H]1CCN(c2nc(-c3onc4c3CCC[C@@]43CCCCC3=O)nc3c2nc(C(F)(F)F)n3-c2ccc3ncc(Br)n3c2)C1] | 1.17 | 726 | C | 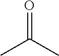 |
| Y-1ba | 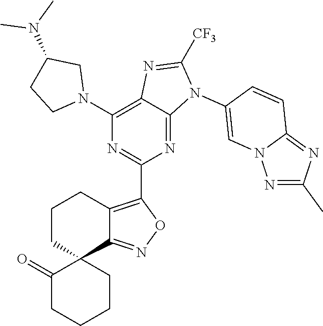 | 1.03 | 635 | C | 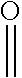 |
| Y-1bb | 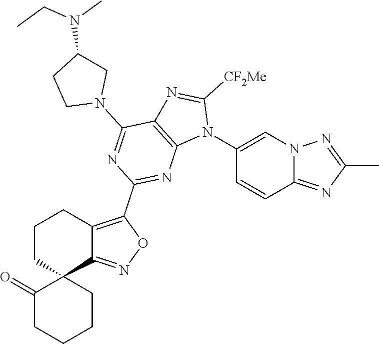 | 1.11 | 644 | A | 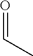 |
| Y-1bc | 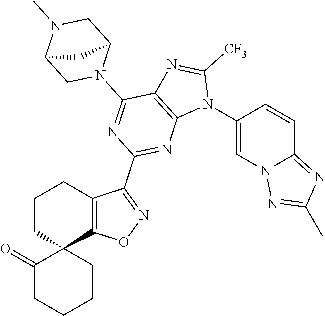 [SMILES: Cc1nc2ccc(-n3c(C(F)(F)F)nc4c(N5C[C@@H]6C[C@H]5CN6C)nc(-c5noc6c5CCC[C@@]65CCCCC5=O)nc43)cn2n1] | 1.12 | 632 | A | 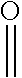 |
| Y-1bd | 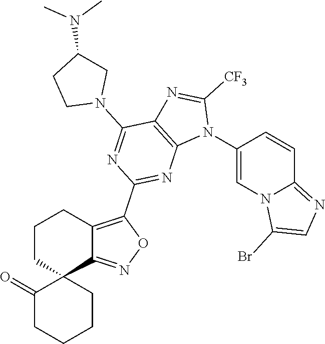 [SMILES: CN(C)[C@H]1CCN(c2nc(-c3onc4c3CCC[C@@]43CCCCC3=O)nc3c2nc(C(F)(F)F)n3-c2ccc3ncc(Br)n3c2)C1] | 1.17 | 698 | A | 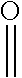 |
| Y-1be | 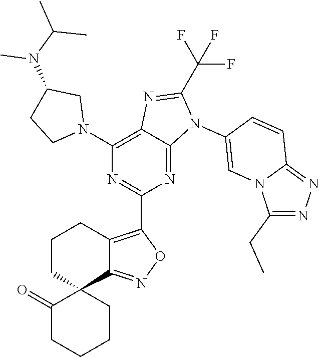 [SMILES: CCc1nnc2ccc(-n3c(C(F)(F)F)nc4c(N5CC[C@H](N(C)C(C)C)C5)nc(-c5onc6c5CCC[C@@]65CCCCC5=O)nc43)cn12] | 1.21 | 677 | A | 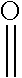 |

### Synthesis of Azapurine

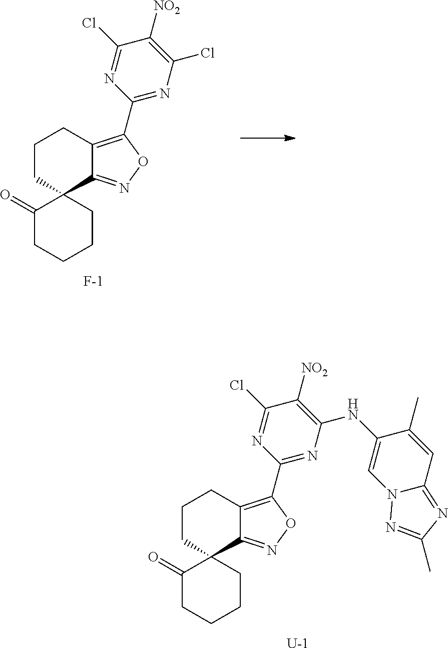

A solution of (7S)-3-(4,6-dichloro-5-nitropyrimidin-2-yl)-5,6-dihydro-4H-spiro[2,1-benzoxazole-7,1′-cyclohexan]-6′-one F-1 (300 mg, 1.0 equiv.) in dry THF (6 mL) is prepared. To this, 2,7-dimethyl-[1,2,4]triazolo[1,5-a]pyridin-6-amine (109.1 mg, 1.0 equiv.) is added and the solution is cooled to −10° C. Then, LiHMDS (1.0M in THF, 2.68 mL, 4.0 equiv.) is added dropwise and the reaction mixture is stirred at −10° C. for 1 hour. The reaction mixture is then extracted with DCM/saturated NaHCO₃ solution. The combined organic layers are dried and the solvent is removed to get a yellow solid. U-1 (HPLC method A, tᵣₑₜ=0.81 min; [M+H]=523).

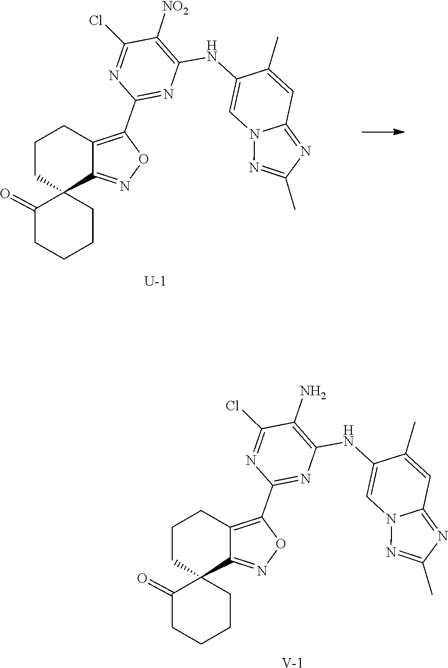

A solution of (7S)-3-[4-chloro-6-({2,7-dimethyl-[1,2,4]triazolo[1,5-a]pyridin-6-yl}amino)-5-nitropyrimidin-2-yl]-5,6-dihydro-4H-spiro[2,1-benzoxazole-7,1′-cyclohexan]-6′-one U-1 (987 mg, 1.0 equiv.) in acetic acid (8 mL) is prepared. To this, iron (543.3 mg, 5.0 equiv.) is added and the reaction mixture is stirred at 40° C. for 1 hour.

The reaction mixture is then diluted in DCM, and saturated NaHCO₃ and K₂CO₃ are added until no more CO₂ is produced. Methanol is added and the reaction mixture is filtered over celite. The phases are separated, and the organic layer is dried over MgSO₄ and concentrated under reduced pressure to give the desired product V-1 (HPLC method A, tᵣₑₜ=0.64 min; [M+H]=493).

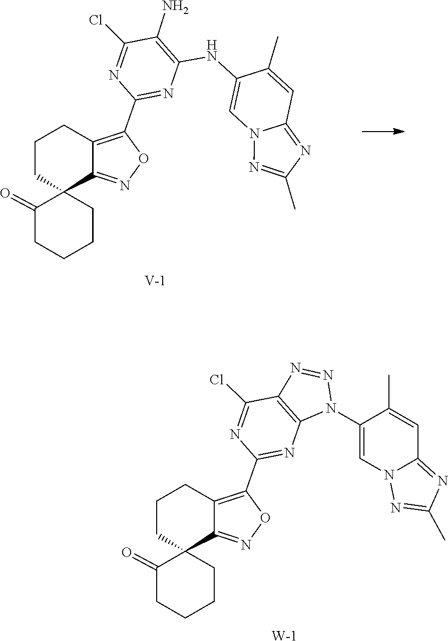

A solution of (7S)-3-[5-amino-4-chloro-6-({2,7-dimethyl-[1,2,4]triazolo[1,5-a]pyridin-6-yl}amino)pyrimidin-2-yl]-5,6-dihydro-4H-spiro[2,1-benzoxazole-7,1′-cyclohexan]-6′-one V-1 (780 mg, 1.0 equiv.) in acetic acid (4 mL) is prepared. The reaction mixture is stirred at 0° C. and a solution of sodium nitrite (180.1 mg, 2.0 equiv.) in water (4 mL) is added dropwise. The reaction mixture is allowed to reach room temperature and is stirred for 1 hour. The reaction mixture is then diluted in DCM, and saturated NaHCO₃ is added. The phases are separated, and the organic layer is dried over MgSO₄ and concentrated under reduced pressure to give the desired product W-1 (HPLC method A, tᵣₑₜ=0.97 min; [M+H]=503).

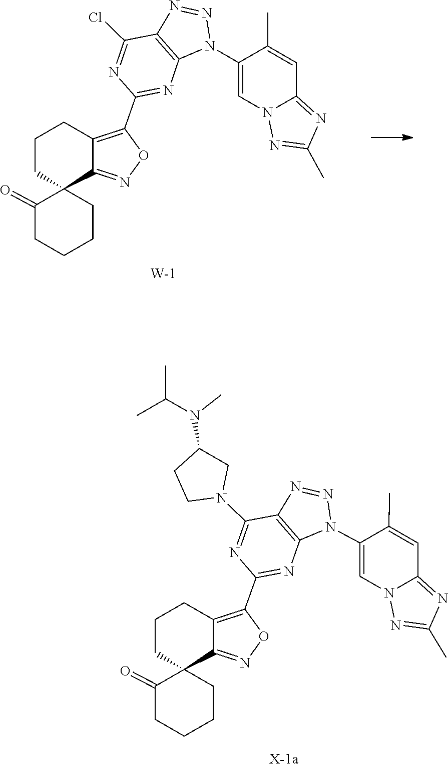

A solution of (7S)-3-(7-chloro-3-{2,7-dimethyl-[1,2,4]triazolo[1,5-a]pyridin-6-yl}-3H-[1,2,3]triazolo[4,5-d]pyrimidin-5-yl)-5,6-dihydro-4H-spiro[2,1-benzoxazole-7,1′-cyclohexan]-6′-one W-1 (100 mg, 1.0 equiv.) and (S)—N-isopropyl-N-methylpyrrolidin-3-amine (48 mg, 0.22 mmol, 1.1 equiv) in NMP (1 mL) is prepared. To this, DIPEA (104.2 μL, 3.0 equiv.) is added and the reaction mixture is shaken at 60° C. 72 h. The reaction mixture is then diluted in DCM, and saturated NaHCO₃ is added. The phases are separated, and the organic layer is dried over MgSO₄ and concentrated under reduced pressure The crude product was purified by RP-chromatography to give the desired product X-1a (HPLC method A, t_{ref}=1.13 min; [M+H]=609).

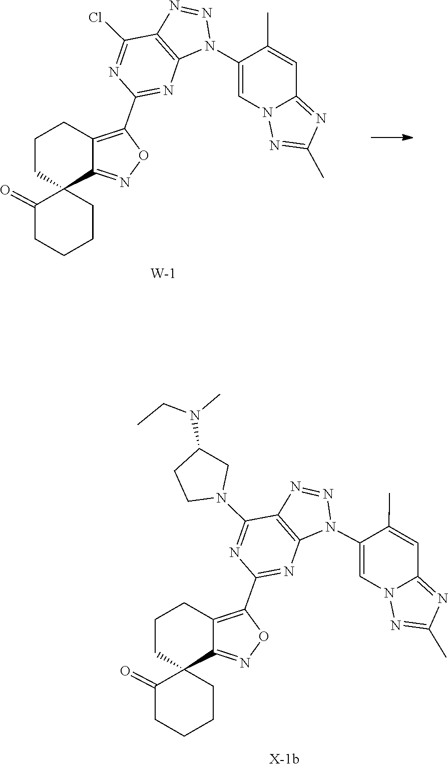

A solution of (7S)-3-(7-chloro-3-{2,7-dimethyl-[1,2,4]triazolo[1,5-a]pyridin-6-yl}-3H-[1,2,3]triazolo[4,5-d]pyrimidin-5-yl)-5,6-dihydro-4H-spiro[2,1-benzoxazole-7,1′-cyclohexan]-6′-one W-1 (100 mg, 1.0 equiv.) and (S)—N-ethyl-N-methylpyrrolidin-3-amine (44 mg, 0.22 mmol, 1.1 equiv) in NMP (1 mL) is prepared. To this, DIPEA (104.2 μL, 3.0 equiv.) is added and the reaction mixture is shaken at 60° C. over the weekend. The reaction mixture is then diluted in DCM, and saturated NaHCO₃ is added. The phases are separated, and the organic layer is dried over MgSO₄ and concentrated under reduced pressure The crude product was purified by RP-chromatography to give the desired product X-1b (HPLC method A, tᵣₑₜ=1.11 min; [M+H]=596).

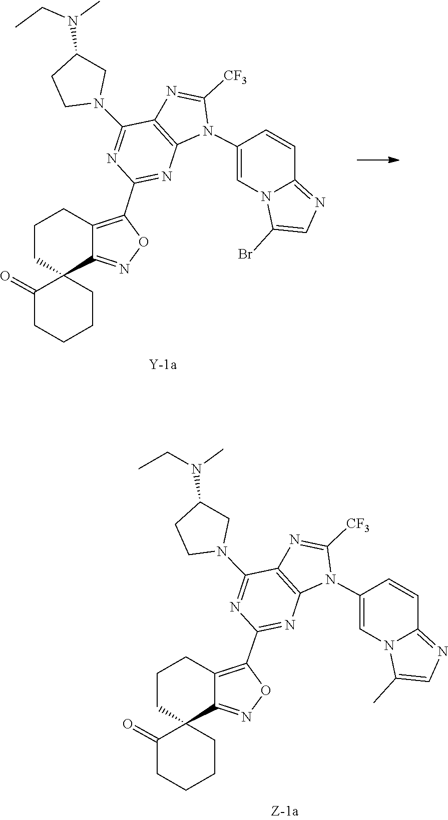 [SMILES: CCN(C)[C@H]1CCN(c2nc(-c3onc4c3CCC[C@@]43CCCCC3=O)nc3c2nc(C(F)(F)F)n3-c2ccc3ncc(Br)n3c2)C1 ; CCN(C)[C@H]1CCN(c2nc(-c3onc4c3CCC[C@@]43CCCCC3=O)nc3c2nc(C(F)(F)F)n3-c2ccc3ncc(C)n3c2)C1]

A solution of Y-1a (179 mg, 0.25 mmol, 1.0 equiv.) in a mixture of dioxane, water, and methanol is added to sodium carbonate (80 mg, 0.75 mmol, 3.0 equiv.) and TetrakisPalladium(0) (58 mg, 0.05 mmol, 0.2 equiv.) in a microwave vial. Trimethylboroxine (35.47 μL, 0.25 mmol, 1.0 equiv.) is then added. The orange/brown reaction mixture is degassed with argon and heated in the microwave to 120° C. for 4 hours. The reaction mixture, which has turned darker, is heated again to 120° C. for 2 hours in the microwave. After filtration, the crude product was purified by RP-chromatography to give the desired product Z-1a (HPLC method A, tᵣₑₜ=1.08 min; [M+H]=648).

The following intermediate compounds Z-1 (Table 23) are obtained analogously. For vinylations, 2,4,6-trivinylcyclotriboroxine pyridine complex is used. For cyanations, zinc cyanide is used.

TABLE 23

| | | tᵣₑₜ | | HPLC |
|---|---|---|---|---|
| # | Structure | [min] | [M + H]⁺ | method |
| Z-1a | 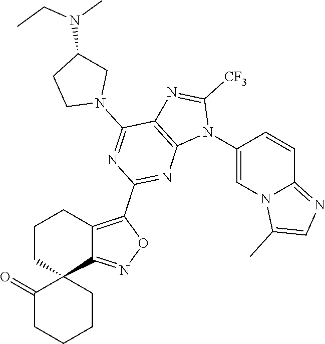 [SMILES: CCN(C)[C@H]1CCN(c2nc(-c3onc4c3CCC[C@@]43CCCCC3=O)nc3c2nc(C(F)(F)F)n3-c2ccc3ncc(C)n3c2)C1] | 1.11 | 634 | A |
| Z-1b | 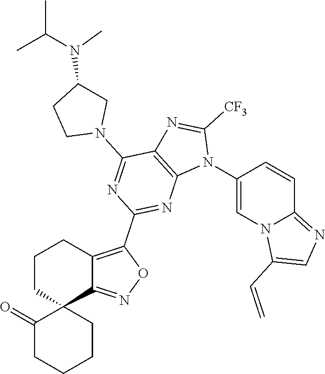 [SMILES: C=Cc1cnc2ccc(-n3c(C(F)(F)F)nc4c(N5CC[C@H](N(C)C(C)C)C5)nc(-c5onc6c5CCC[C@@]65CCCCC5=O)nc43)cn12] | 1.13 | 674 | A |
| Z-1c | 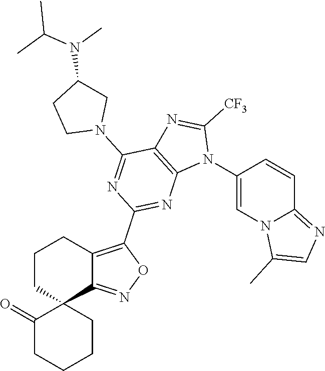 [SMILES: Cc1cnc2ccc(-n3c(C(F)(F)F)nc4c(N5CC[C@H](N(C)C(C)C)C5)nc(-c5onc6c5CCC[C@@]65CCCCC5=O)nc43)cn12] | 1.08 | 661 | A |
| Z-1d | 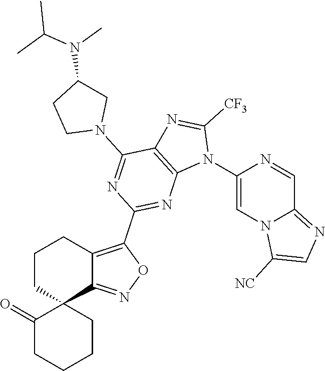 [SMILES: CC(C)N(C)[C@H]1CCN(c2nc(-c3onc4c3CCC[C@@]43CCCCC3=O)nc3c2nc(C(F)(F)F)n3-c2cn3c(C#N)cnc3cn2)C1] | 1.01 | 674 | A |
| Z-1e | 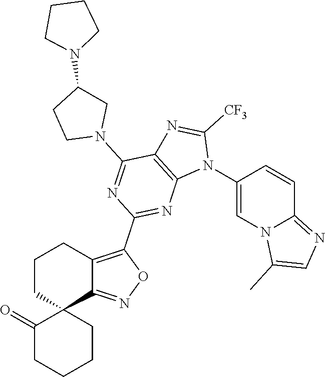 [SMILES: Cc1cnc2ccc(-n3c(C(F)(F)F)nc4c(N5CC[C@H](N6CCCC6)C5)nc(-c5onc6c5CCC[C@@]65CCCCC5=O)nc43)cn12] | 1.08 | 646 | A |
| Z-1f | 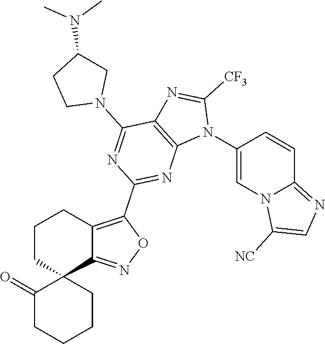 [SMILES: CN(C)[C@H]1CCN(c2nc(-c3onc4c3CCC[C@@]43CCCCC3=O)nc3c2nc(C(F)(F)F)n3-c2ccc3ncc(C#N)n3c2)C1] | 1.05 | 645 | A |
| Z-1g | 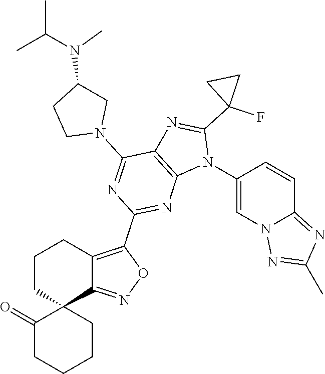 [SMILES: Cc1nc2ccc(-n3c(C4(F)CC4)nc4c(N5CC[C@H](N(C)C(C)C)C5)nc(-c5onc6c5CCC[C@@]65CCCCC5=O)nc43)cn2n1] | 1.12 | 653 | A |
| Z-1h | 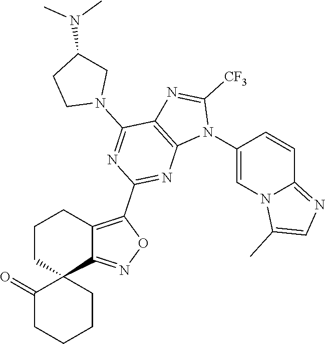 [SMILES: Cc1cnc2ccc(-n3c(C(F)(F)F)nc4c(N5CC[C@H](N(C)C)C5)nc(-c5onc6c5CCC[C@@]65CCCCC5=O)nc43)cn12] | 1.09 | 634 | A |

### Gewald Reaction

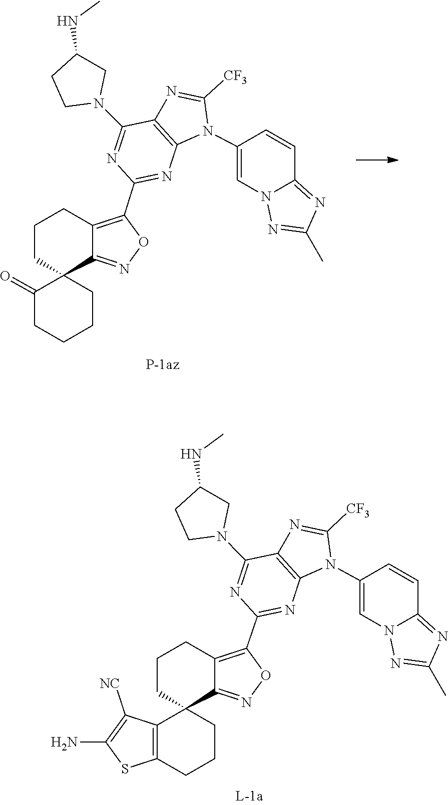 [SMILES: CN[C@H]1CCN(c2nc(-c3onc4c3CCC[C@@]43CCCCC3=O)nc3c2nc(C(F)(F)F)n3-c2ccc3nc(C)nn3c2)C1 ; CN[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1]

A solution of P-1az (420 mg, 1.0 equiv.) in anhydrous ethanol (4.2 mL) and anhydrous toluene (2.1 mL) is degassed with argon. To this, malononitrile (182.5 mg, 4.0 equiv.), sulfur (86.8 mg, 4.0 equiv.), and ammonium acetate (260.8 mg, 5.0 equiv.) are added. The reaction mixture is stirred at 55° C. for 3 hours.

The reaction mixture is then allowed to cool down to room temperature and washed with 0.5 M sodium hydroxide solution (3×40 mL). The organic phase is dried over magnesium sulfate, filtered, and the solvent is evaporated. The crude product is purified by silica gel chromatography (DCM/MeOH, 0-10% MeOH, 60 mL/min) to give the desired product L-1a (HPLC method A, tᵣₑₜ=1.00 min; [M+H]=701).

The following intermediate compounds L-1 (Table 24) are obtained analogously.

TABLE 24

| | | tᵣₑₜ | | HPLC |
|---|---|---|---|---|
| # | structure | [min] | [M + H]⁺ | method |
| L-1b | 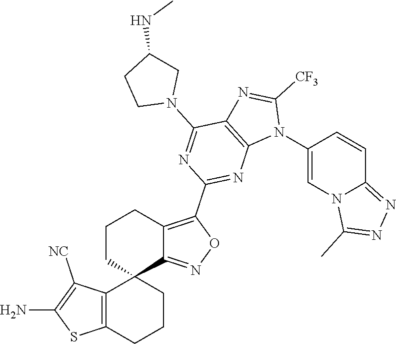 [SMILES: CN[C@H]1CCN(c2nc(-c3onc4c3CCC[C@@]43CCCc4sc(N)c(C#N)c43)nc3c2nc(C(F)(F)F)n3-c2ccc3nnc(C)n3c2)C1] | 0.85 | 621 | A |
| L-1c | 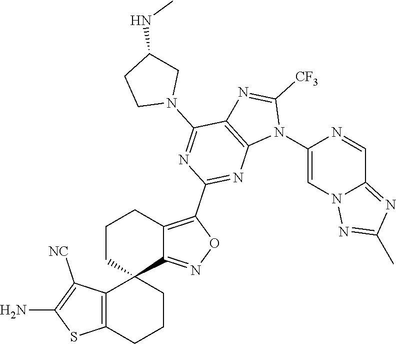 [SMILES: CN[C@H]1CCN(c2nc(-c3onc4c3CCC[C@@]43CCCc4sc(N)c(C#N)c43)nc3c2nc(C(F)(F)F)n3-c2cn3nc(C)nc3cn2)C1] | 1.65 | 744 | A |
| L-1d | 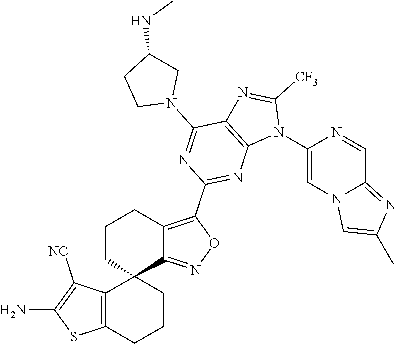 [SMILES: CN[C@H]1CCN(c2nc(-c3onc4c3CCC[C@@]43CCCc4sc(N)c(C#N)c43)nc3c2nc(C(F)(F)F)n3-c2cn3cc(C)nc3cn2)C1] | 0.99 | 701 | A |
| L-1e | 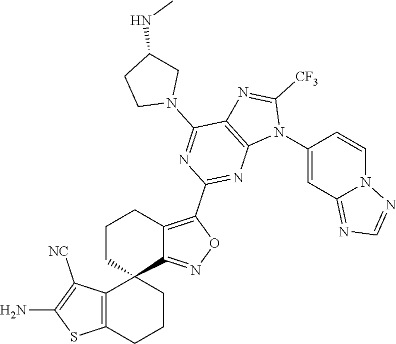 [SMILES: CN[C@H]1CCN(c2nc(-c3onc4c3CCC[C@@]43CCCc4sc(N)c(C#N)c43)nc3c2nc(C(F)(F)F)n3-c2ccn3ncnc3c2)C1] | 1.01 | 687 | A |
| L-1f | 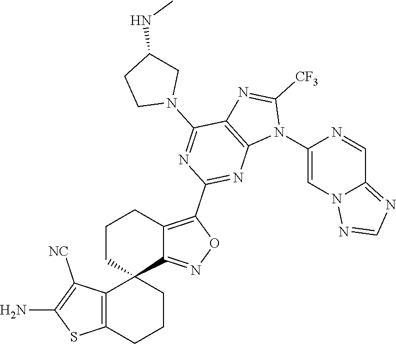 [SMILES: CN[C@H]1CCN(c2nc(-c3onc4c3CCC[C@@]43CCCc4sc(N)c(C#N)c43)nc3c2nc(C(F)(F)F)n3-c2cn3ncnc3cn2)C1] | 1.01 | 688 | A |
| L-1g | 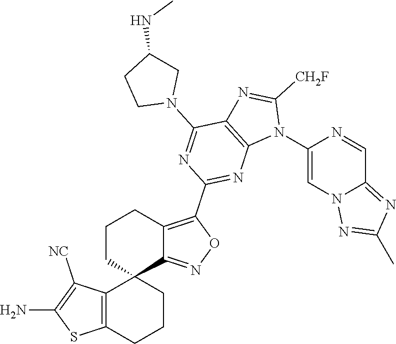 | 0.95 | 666 | A |
| L-1h | 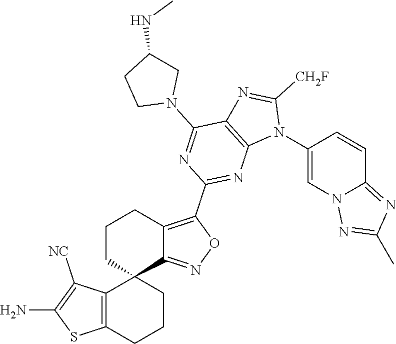 | 1.29 | 665 | A |
| L-1i | 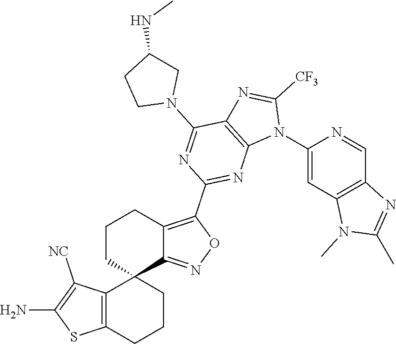 [SMILES: CN[C@H]1CCN(c2nc(-c3onc4c3CCC[C@@]43CCCc4sc(N)c(C#N)c43)nc3c2nc(C(F)(F)F)n3-c2cc3c(cn2)nc(C)n3C)C1] | 1.00 | 715 | A |
| L-1j | 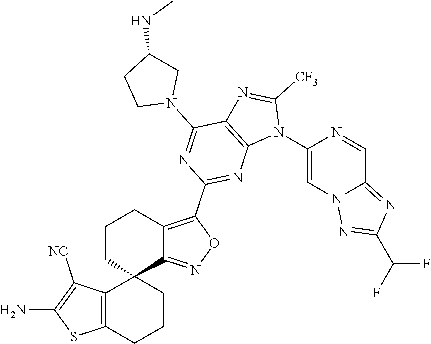 [SMILES: CN[C@H]1CCN(c2nc(-c3onc4c3CCC[C@@]43CCCc4sc(N)c(C#N)c43)nc3c2nc(C(F)(F)F)n3-c2cn3nc(C(F)F)nc3cn2)C1] | 1.00 | 738 | 4 |
| L-1k | 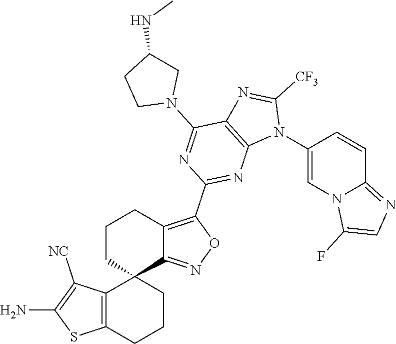 [SMILES: CN[C@H]1CCN(c2nc(-c3onc4c3CCC[C@@]43CCCc4sc(N)c(C#N)c43)nc3c2nc(C(F)(F)F)n3-c2ccc3ncc(F)n3c2)C1] | 1.05 | 704 | 4 |
| L-1l | 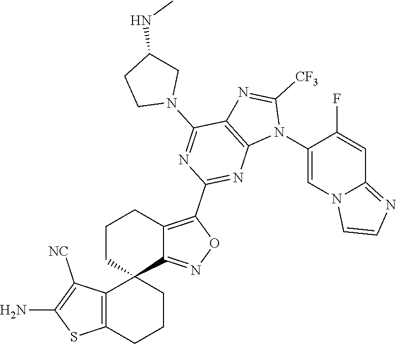 [SMILES: CN[C@H]1CCN(c2nc(-c3onc4c3CCC[C@@]43CCCc4sc(N)c(C#N)c43)nc3c2nc(C(F)(F)F)n3-c2cn3ccnc3cc2F)C1] | 1.02 | 704 | A |
| L-1m | 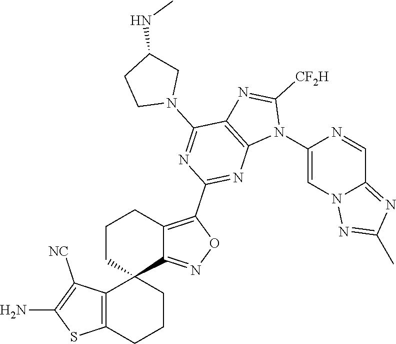 [SMILES: CN[C@H]1CCN(c2nc(-c3onc4c3CCC[C@@]43CCCc4sc(N)c(C#N)c43)nc3c2nc(C(F)F)n3-c2cn3nc(C)nc3cn2)C1] | 1.00 | 684 | A |
| L-1n | 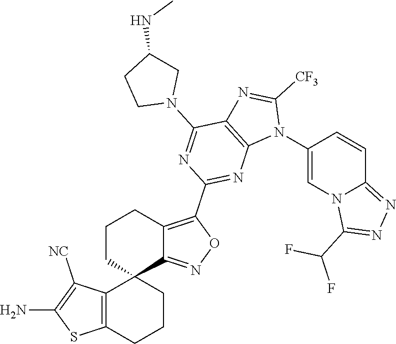 [SMILES: CN[C@H]1CCN(c2nc(-c3onc4c3CCC[C@@]43CCCc4sc(N)c(C#N)c43)nc3c2nc(C(F)(F)F)n3-c2ccc3nnc(C(F)F)n3c2)C1] | 1.01 | 737 | A |

### Synthesis of Final Compounds II

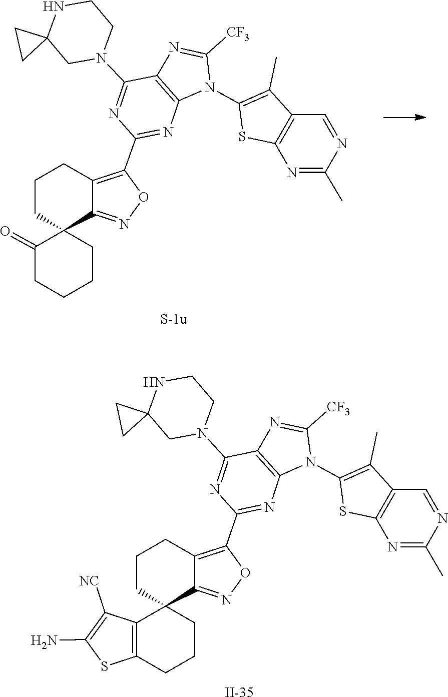

A solution of S-1u (38 mg, 0.06 mmol, 1.0 equiv.) in dry ethanol (1 mL) is added to a mixture of malononitrile (7.6 mg, 0.12 mmol, 2.0 equiv.), sulfur (3.6 mg, 0.12 mmol, 2.0 equiv.), and ammonium acetate (8.8 mg, 0.12 mmol, 2.0 equiv.). The reaction mixture is stirred at 60° C. for 3 hours. HPLC-MS shows full conversion of the starting material to the product after 3 hours. The reaction mixture is directly loaded onto Isolute and purified by RP-chromatography to give the desired product 11-35 (H PLC method A, tᵣₑₜ=1.54 min; [M+H]=744).

The following final compounds 11 (Table 25) are obtained analogously from the respective compounds S-1, T-1, Y-1, or Z-1. The crude product is purified by chromatography if necessary.

TABLE 25

| | | tᵣₑₜ | | HPLC |
|---|---|---|---|---|
| # | structure | [min] | [M + H]⁺ | method |
| II-1 | 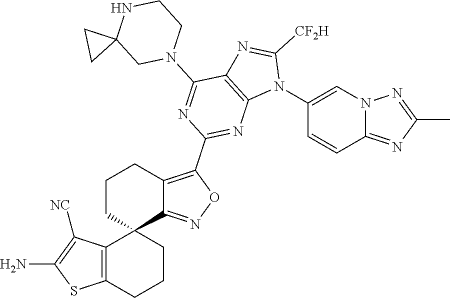 [SMILES: Cc1nc2ccc(-n3c(C(F)F)nc4c(N5CCNC6(CC6)C5)nc(-c5onc6c5CCC[C@@]65CCCc6sc(N)c(C#N)c65)nc43)cn2n1] | 1.00 | 695 | B |
| II-2 | 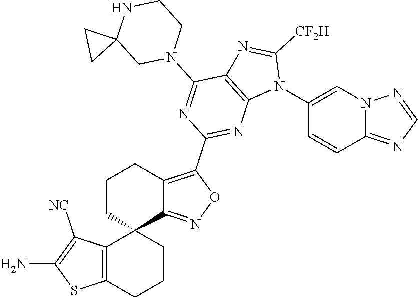 [SMILES: N#Cc1c(N)sc2c1[C@@]1(CCC2)CCCc2c1noc2-c1nc(N2CCNC3(CC3)C2)c2nc(C(F)F)n(-c3ccc4ncnn4c3)c2n1] | 1.01 | 681 | B |
| II-3 | 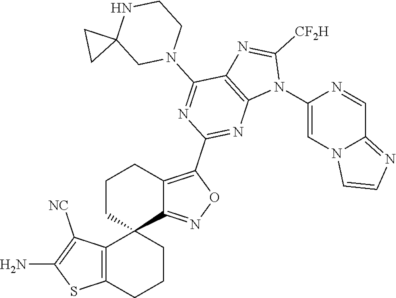 [SMILES: N#Cc1c(N)sc2c1[C@@]1(CCC2)CCCc2c1noc2-c1nc(N2CCNC3(CC3)C2)c2nc(C(F)F)n(-c3cn4ccnc4cn3)c2n1] | 1.29 | 681 | B |
| II-4 | 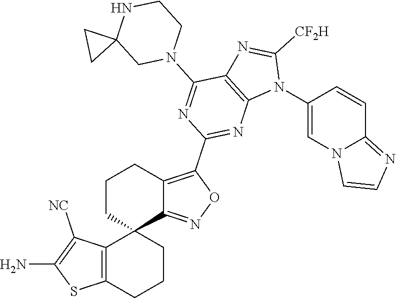 [SMILES: N#Cc1c(N)sc2c1[C@@]1(CCC2)CCCc2c1noc2-c1nc(N2CCNC3(CC3)C2)c2nc(C(F)F)n(-c3ccc4nccn4c3)c2n1] | 1.28 | 680 | B |
| II-5 | 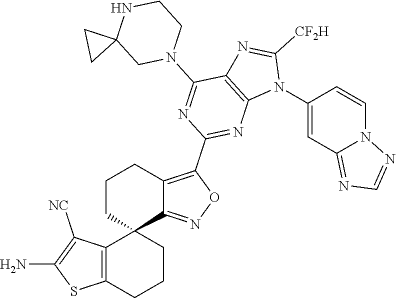 [SMILES: N#Cc1c(N)sc2c1[C@@]1(CCC2)CCCc2c1noc2-c1nc(N2CCNC3(CC3)C2)c2nc(C(F)F)n(-c3ccn4ncnc4c3)c2n1] | 1.11 | 681 | B |
| II-6 | 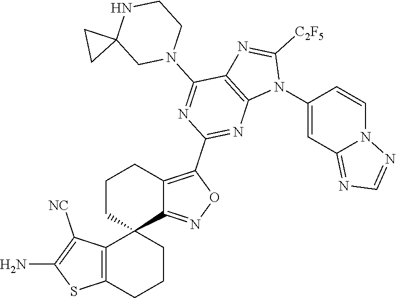 | 1.08 | 749 | B |
| II-7 | 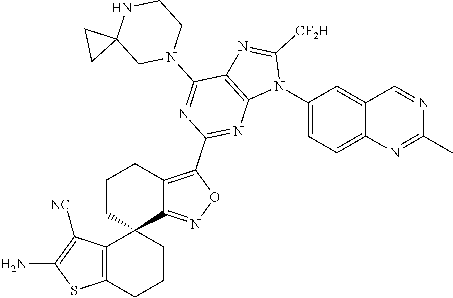 [SMILES: Cc1ncc2cc(-n3c(C(F)F)nc4c(N5CCNC6(CC6)C5)nc(-c5onc6c5CCC[C@@]65CCCc6sc(N)c(C#N)c65)nc43)ccc2n1] | 1.45 | 724 | B |
| II-8 | 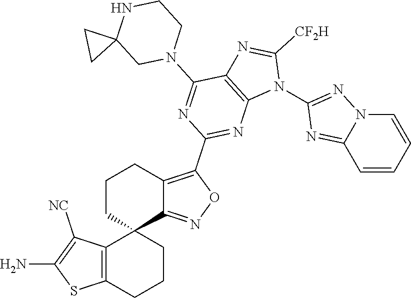 [SMILES: N#Cc1c(N)sc2c1[C@@]1(CCC2)CCCc2c1noc2-c1nc(N2CCNC3(CC3)C2)c2nc(C(F)F)n(-c3nc4ccccn4n3)c2n1] | 1.37 | 681 | B |
| II-9 | 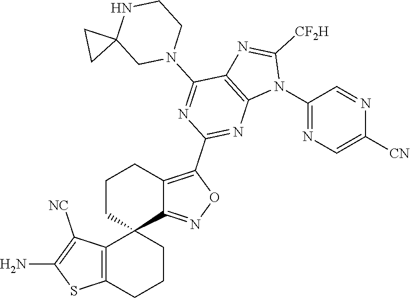 [SMILES: N#Cc1cnc(-n2c(C(F)F)nc3c(N4CCNC5(CC5)C4)nc(-c4onc5c4CCC[C@@]54CCCc5sc(N)c(C#N)c54)nc32)cn1] | 1.46 | 667 | B |
| II-10 | 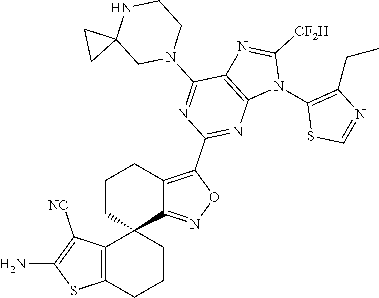 [SMILES: CCc1ncsc1-n1c(C(F)F)nc2c(N3CCNC4(CC4)C3)nc(-c3onc4c3CCC[C@@]43CCCc4sc(N)c(C#N)c43)nc21] | 1.44 | 675 | B |
| II-11 | 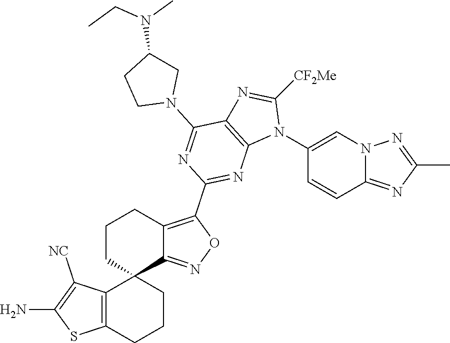 | 1.54 | 725 | B |
| II-12 | 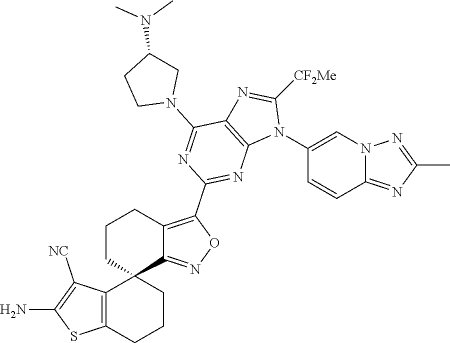 | 1.48 | 711 | B |
| II-13 | 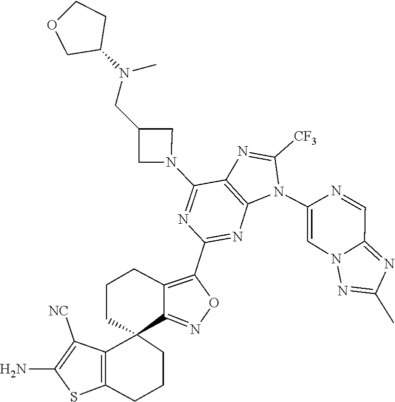 [SMILES: Cc1nc2cnc(-n3c(C(F)(F)F)nc4c(N5CC(CN(C)[C@H]6CCOC6)C5)nc(-c5onc6c5CCC[C@@]65CCCc6sc(N)c(C#N)c65)nc43)cn2n1] | 0.99 | 772 | B |
| II-14 | 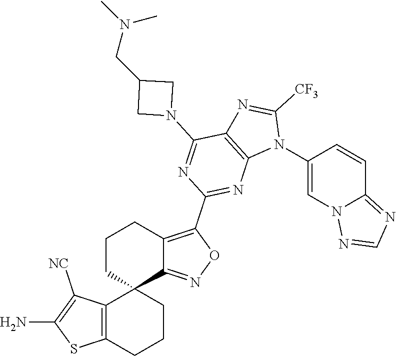 [SMILES: CN(C)CC1CN(c2nc(-c3onc4c3CCC[C@@]43CCCc4sc(N)c(C#N)c43)nc3c2nc(C(F)(F)F)n3-c2ccc3ncnn3c2)C1] | 0.97 | 701 | B |
| II-15 | 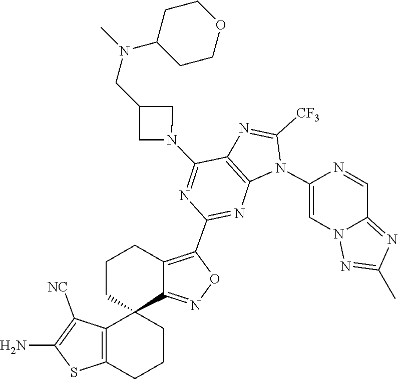 [SMILES: Cc1nc2cnc(-n3c(C(F)(F)F)nc4c(N5CC(CN(C)C6CCOCC6)C5)nc(-c5onc6c5CCC[C@@]65CCCc6sc(N)c(C#N)c65)nc43)cn2n1] | 1.05 | 786 | B |
| II-16 | 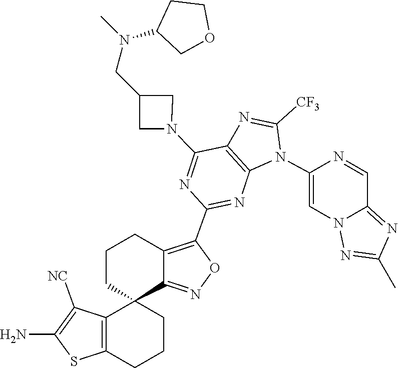 | 0.99 | 772 | B |
| II-17 | 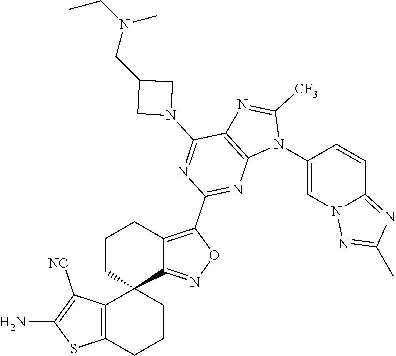 [SMILES: CCN(C)CC1CN(c2nc(-c3onc4c3CCC[C@@]43CCCc4sc(N)c(C#N)c43)nc3c2nc(C(F)(F)F)n3-c2ccc3nc(C)nn3c2)C1] | 1.10 | 729 | B |
| II-18 | 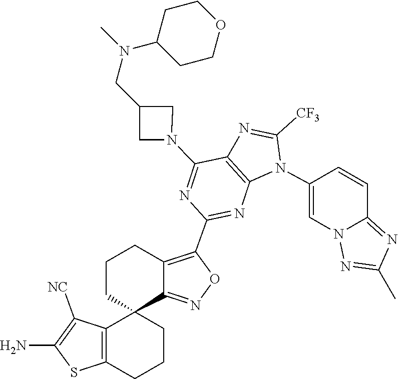 [SMILES: Cc1nc2ccc(-n3c(C(F)(F)F)nc4c(N5CC(CN(C)C6CCOCC6)C5)nc(-c5onc6c5CCC[C@@]65CCCc6sc(N)c(C#N)c65)nc43)cn2n1] | 0.98 | 785 | B |
| II-19 | 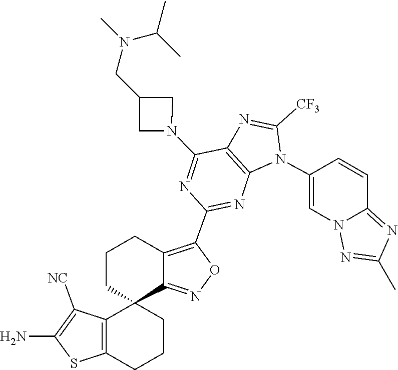 [SMILES: Cc1nc2ccc(-n3c(C(F)(F)F)nc4c(N5CC(CN(C)C(C)C)C5)nc(-c5onc6c5CCC[C@@]65CCCc6sc(N)c(C#N)c65)nc43)cn2n1] | 1.05 | 743 | B |
| II-20 | 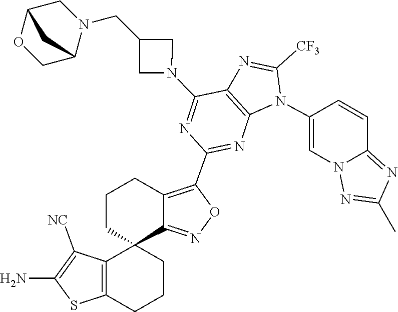 [SMILES: Cc1nc2ccc(-n3c(C(F)(F)F)nc4c(N5CC(CN6C[C@@H]7C[C@H]6CO7)C5)nc(-c5onc6c5CCC[C@@]65CCCc6sc(N)c(C#N)c65)nc43)cn2n1] | 0.93 | 769 | B |
| II-21 | 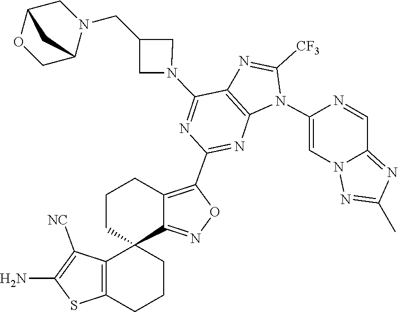 [SMILES: Cc1nc2cnc(-n3c(C(F)(F)F)nc4c(N5CC(CN6C[C@@H]7C[C@H]6CO7)C5)nc(-c5onc6c5CCC[C@@]65CCCc6sc(N)c(C#N)c65)nc43)cn2n1] | 0.96 | 770 | B |
| II-22 | 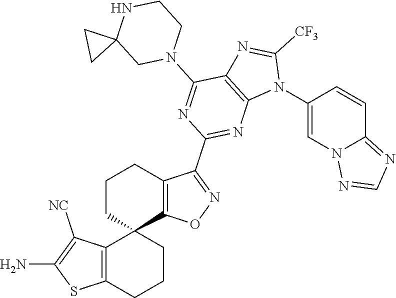 [SMILES: N#Cc1c(N)sc2c1[C@@]1(CCC2)CCCc2c(-c3nc(N4CCNC5(CC5)C4)c4nc(C(F)(F)F)n(-c5ccc6ncnn6c5)c4n3)noc21] | 1.34 | 699 | B |
| II-23 | 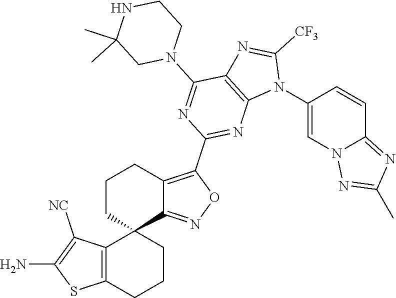 [SMILES: Cc1nc2ccc(-n3c(C(F)(F)F)nc4c(N5CCNC(C)(C)C5)nc(-c5onc6c5CCC[C@@]65CCCc6sc(N)c(C#N)c65)nc43)cn2n1] | 1.40 | 715 | B |
| II-24 | 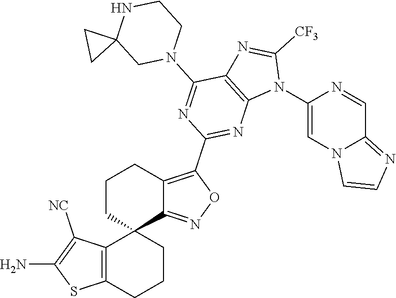 [SMILES: N#Cc1c(N)sc2c1[C@@]1(CCC2)CCCc2c1noc2-c1nc(N2CCNC3(CC3)C2)c2nc(C(F)(F)F)n(-c3cn4ccnc4cn3)c2n1] | 1.33 | 699 | B |
| II-25 | 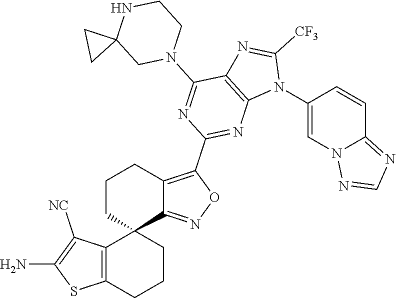 [SMILES: N#Cc1c(N)sc2c1[C@@]1(CCC2)CCCc2c1noc2-c1nc(N2CCNC3(CC3)C2)c2nc(C(F)(F)F)n(-c3ccc4ncnn4c3)c2n1] | 1.34 | 699 | B |
| II-26 | 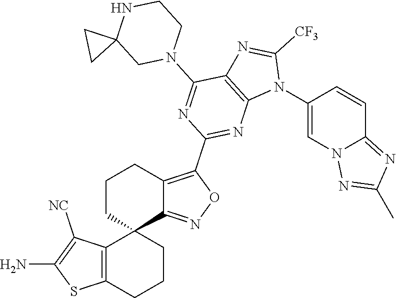 [SMILES: Cc1nc2ccc(-n3c(C(F)(F)F)nc4c(N5CCNC6(CC6)C5)nc(-c5onc6c5CCC[C@@]65CCCc6sc(N)c(C#N)c65)nc43)cn2n1] | 1.37 | 713 | B |
| II-27 | 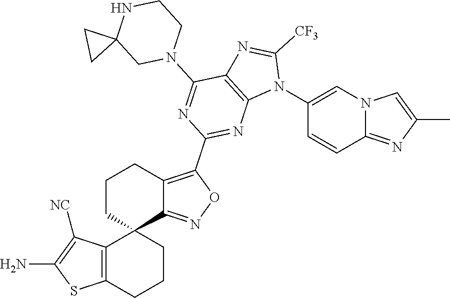 [SMILES: Cc1cn2cc(-n3c(C(F)(F)F)nc4c(N5CCNC6(CC6)C5)nc(-c5onc6c5CCC[C@@]65CCCc6sc(N)c(C#N)c65)nc43)ccc2n1] | 1.37 | 712 | B |
| II-28 | 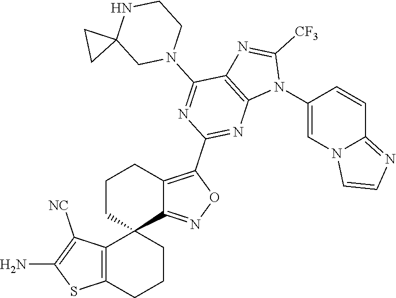 [SMILES: N#Cc1c(N)sc2c1[C@@]1(CCC2)CCCc2c1noc2-c1nc(N2CCNC3(CC3)C2)c2nc(C(F)(F)F)n(-c3ccc4nccn4c3)c2n1] | 0.95 | 698 | B |
| II-29 | 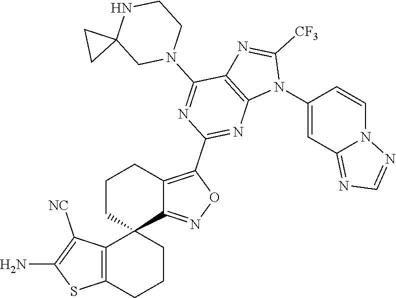 [SMILES: N#Cc1c(N)sc2c1[C@@]1(CCC2)CCCc2c1noc2-c1nc(N2CCNC3(CC3)C2)c2nc(C(F)(F)F)n(-c3ccn4ncnc4c3)c2n1] | 1.35 | 699 | B |
| II-30 | 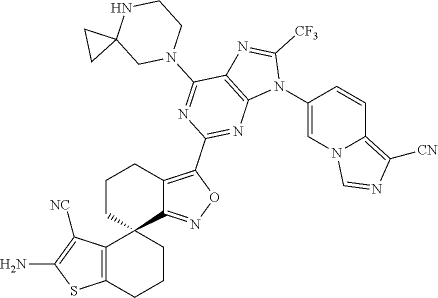 [SMILES: N#Cc1c(N)sc2c1[C@@]1(CCC2)CCCc2c1noc2-c1nc(N2CCNC3(CC3)C2)c2nc(C(F)(F)F)n(-c3ccc4c(C#N)ncn4c3)c2n1] | 1.41 | 723 | B |
| II-31 | 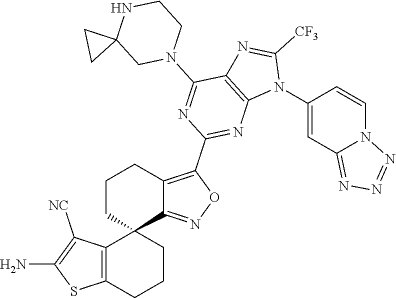 [SMILES: N#Cc1c(N)sc2c1[C@@]1(CCC2)CCCc2c1noc2-c1nc(N2CCNC3(CC3)C2)c2nc(C(F)(F)F)n(-c3ccn4nnnc4c3)c2n1] | 1.37 | 700 | B |
| II-32 | 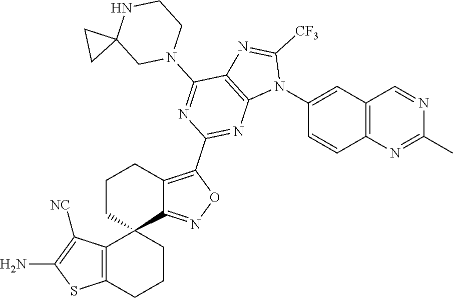 [SMILES: Cc1ncc2cc(-n3c(C(F)(F)F)nc4c(N5CCNC6(CC6)C5)nc(-c5onc6c5CCC[C@@]65CCCc6sc(N)c(C#N)c65)nc43)ccc2n1] | 1.45 | 724 | B |
| II-33 | 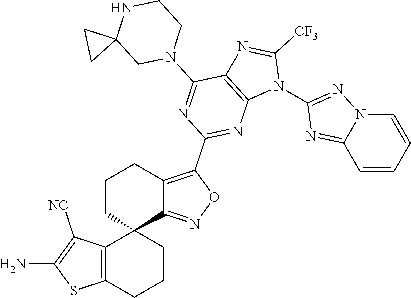 [SMILES: N#Cc1c(N)sc2c1[C@@]1(CCC2)CCCc2c1noc2-c1nc(N2CCNC3(CC3)C2)c2nc(C(F)(F)F)n(-c3nc4ccccn4n3)c2n1] | 1.04 | 699 | B |
| II-34 | 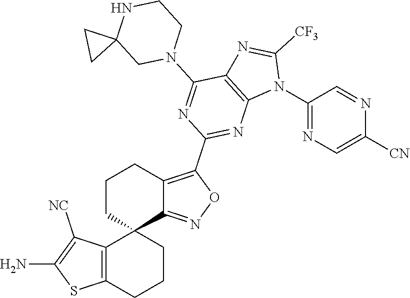 [SMILES: N#Cc1cnc(-n2c(C(F)(F)F)nc3c(N4CCNC5(CC5)C4)nc(-c4onc5c4CCC[C@@]54CCCc5sc(N)c(C#N)c54)nc32)cn1] | 1.511 | 685 | B |
| II-35 | 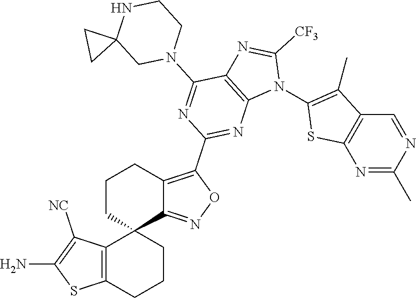 [SMILES: Cc1ncc2c(C)c(-n3c(C(F)(F)F)nc4c(N5CCNC6(CC6)C5)nc(-c5onc6c5CCC[C@@]65CCCc6sc(N)c(C#N)c65)nc43)sc2n1] | 1.54 | 744 | B |
| II-36 | 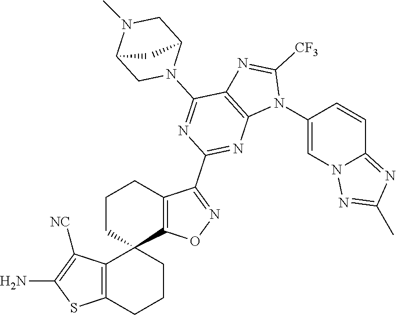 [SMILES: Cc1nc2ccc(-n3c(C(F)(F)F)nc4c(N5C[C@@H]6C[C@H]5CN6C)nc(-c5noc6c5CCC[C@@]65CCCc6sc(N)c(C#N)c65)nc43)cn2n1] | 1.41 | 713 | B |
| II-37 | 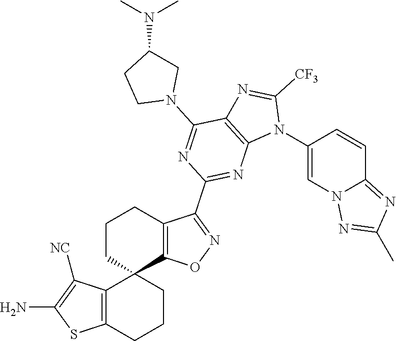 [SMILES: Cc1nc2ccc(-n3c(C(F)(F)F)nc4c(N5CC[C@H](N(C)C)C5)nc(-c5noc6c5CCC[C@@]65CCCc6sc(N)c(C#N)c65)nc43)cn2n1] | 1.45 | 715 | B |
| II-38 | 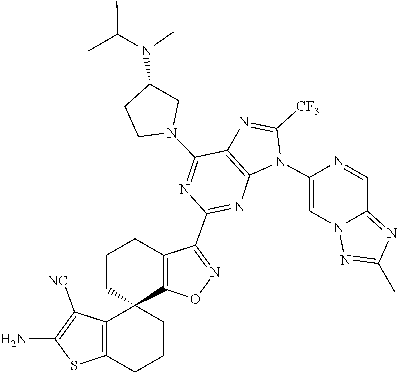 [SMILES: Cc1nc2cnc(-n3c(C(F)(F)F)nc4c(N5CC[C@H](N(C)C(C)C)C5)nc(-c5noc6c5CCC[C@@]65CCCc6sc(N)c(C#N)c65)nc43)cn2n1] | 1.61 | 744 | B |
| II-39 | 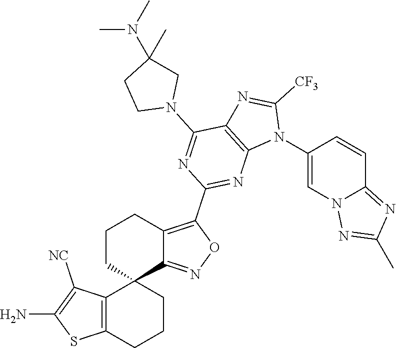 [SMILES: Cc1nc2ccc(-n3c(C(F)(F)F)nc4c(N5CCC(C)(N(C)C)C5)nc(-c5onc6c5CCC[C@@]65CCCc6sc(N)c(C#N)c65)nc43)cn2n1] | 1.53 | 729 | B |
| II-40 | 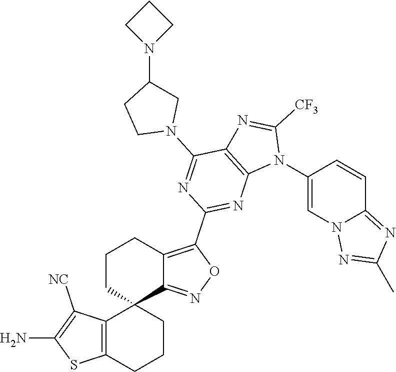 [SMILES: Cc1nc2ccc(-n3c(C(F)(F)F)nc4c(N5CCC(N6CCC6)C5)nc(-c5onc6c5CCC[C@@]65CCCc6sc(N)c(C#N)c65)nc43)cn2n1] | 1.53 | 727 | B |
| II-41 | 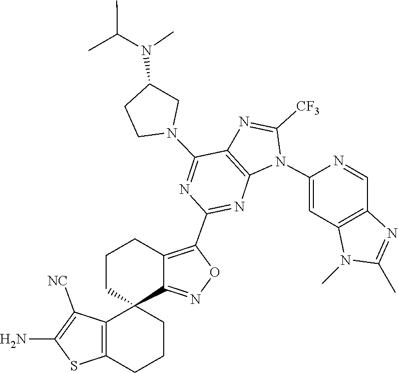 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] | 1.039 | 729 | B |
| II-42 | 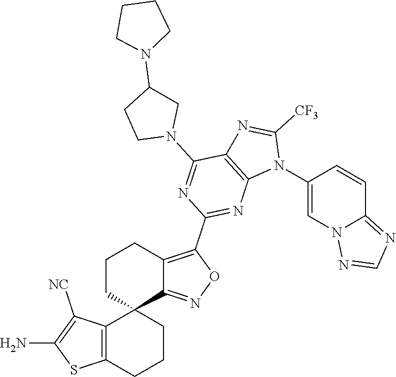 [SMILES: N#Cc1c(N)sc2c1[C@@]1(CCC2)CCCc2c1noc2-c1nc(N2CCC(N3CCCC3)C2)c2nc(C(F)(F)F)n(-c3ccc4ncnn4c3)c2n1] | 1.521 | 727 | B |
| II-43 | 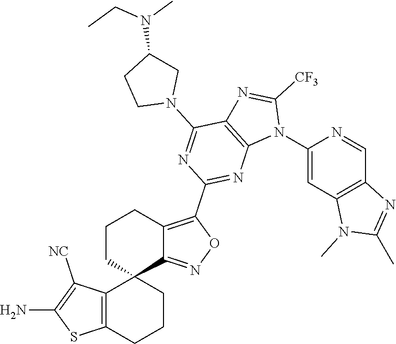 [SMILES: CCN(C)[C@H]1CCN(c2nc(-c3onc4c3CCC[C@@]43CCCc4sc(N)c(C#N)c43)nc3c2nc(C(F)(F)F)n3-c2cc3c(cn2)nc(C)n3C)C1] | 1.491 | 743 | B |
| II-44 | 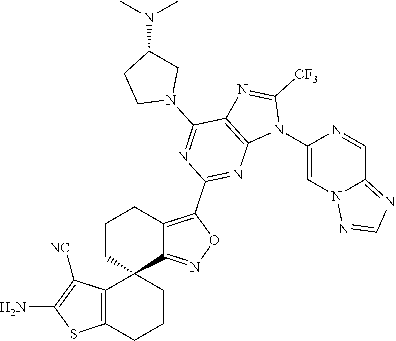 [SMILES: CN(C)[C@H]1CCN(c2nc(-c3onc4c3CCC[C@@]43CCCc4sc(N)c(C#N)c43)nc3c2nc(C(F)(F)F)n3-c2cn3ncnc3cn2)C1] | 1.431 | 702 | B |
| II-45 | 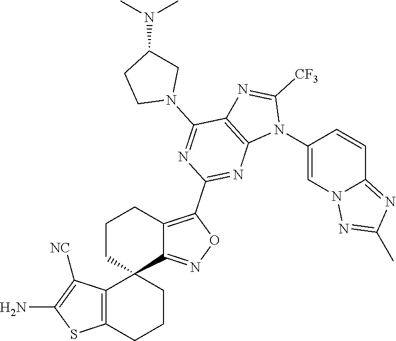 [SMILES: Cc1nc2ccc(-n3c(C(F)(F)F)nc4c(N5CC[C@H](N(C)C)C5)nc(-c5onc6c5CCC[C@@]65CCCc6sc(N)c(C#N)c65)nc43)cn2n1] | 1.46 | 715 | B |
| II-46 | 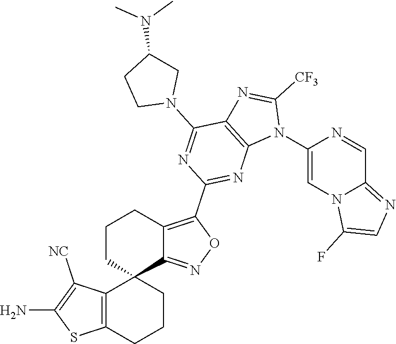 [SMILES: CN(C)[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] | 1.491 | 719 | B |
| II-47 | 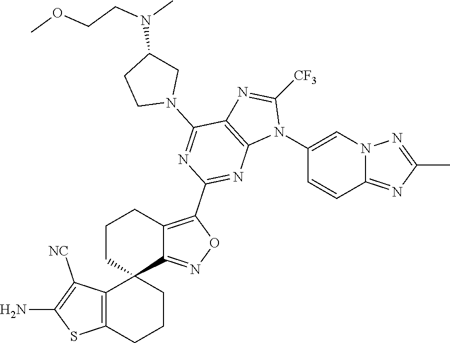 [SMILES: COCCN(C)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1] | 1.481 | 759 | B |
| II-48 | 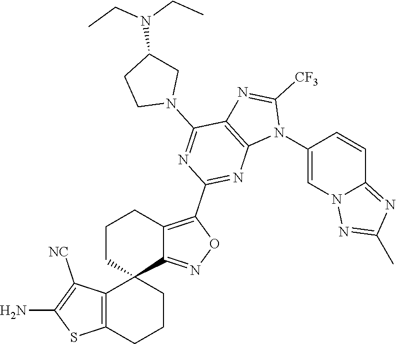 [SMILES: CCN(CC)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1] | 1.61 | 743 | B |
| II-49 | 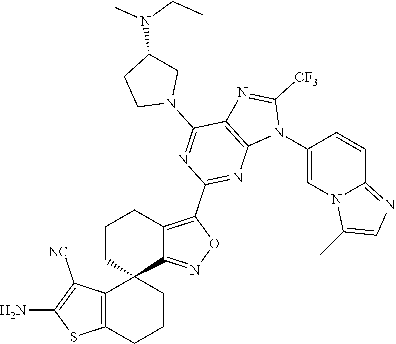 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3ncc(C)n3c2)C1] | 1.51 | 728 | B |
| II-50 | 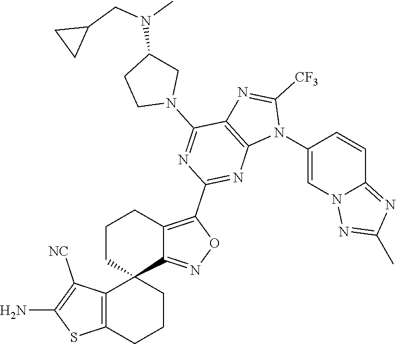 [SMILES: Cc1nc2ccc(-n3c(C(F)(F)F)nc4c(N5CC[C@H](N(C)CC6CC6)C5)nc(-c5onc6c5CCC[C@@]65CCCc6sc(N)c(C#N)c65)nc43)cn2n1] | 1.60 | 755 | B |
| II-51 | 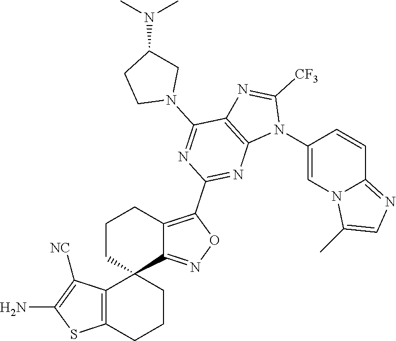 [SMILES: Cc1cnc2ccc(-n3c(C(F)(F)F)nc4c(N5CC[C@H](N(C)C)C5)nc(-c5onc6c5CCC[C@@]65CCCc6sc(N)c(C#N)c65)nc43)cn12] | 1.45 | 714 | B |
| II-52 | 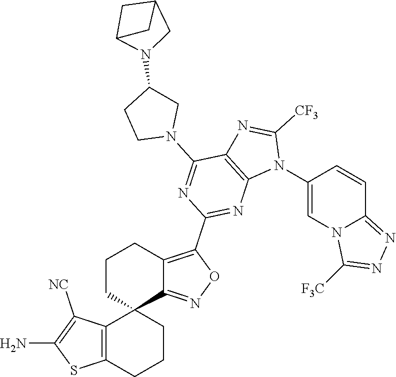 [SMILES: N#Cc1c(N)sc2c1[C@@]1(CCC2)CCCc2c1noc2-c1nc(N2CC[C@H](N3CC4CC3C4)C2)c2nc(C(F)(F)F)n(-c3ccc4nnc(C(F)(F)F)n4c3)c2n1] | 1.61 | 807 | B |
| II-53 | 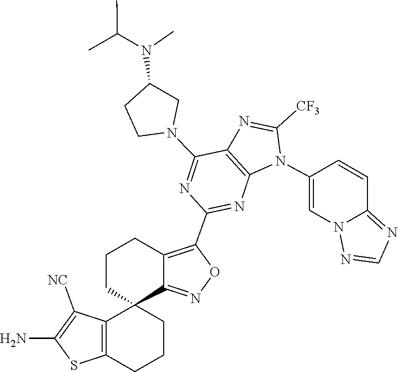 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1] | 1.59 | 729 | B |
| II-54 | 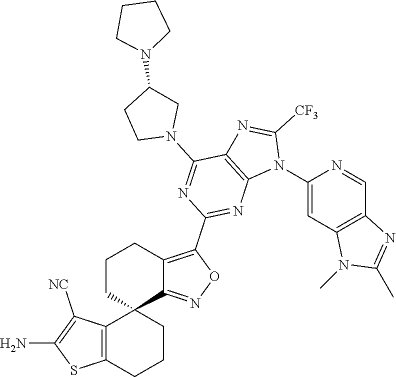 [SMILES: Cc1nc2cnc(-n3c(C(F)(F)F)nc4c(N5CC[C@H](N6CCCC6)C5)nc(-c5onc6c5CCC[C@@]65CCCc6sc(N)c(C#N)c65)nc43)cc2n1C] | 1.53 | 755 | B |
| II-55 | 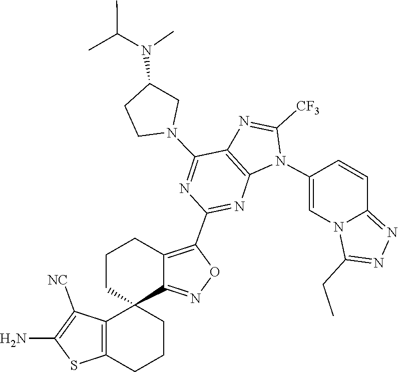 [SMILES: CCc1nnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.56 | 757 | B |
| II-56 | 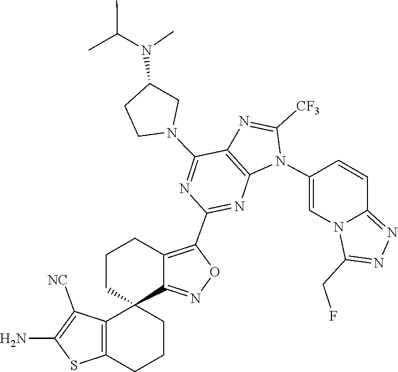 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CF)n3c2)C1] | 1.55 | 761 | B |
| II-57 | 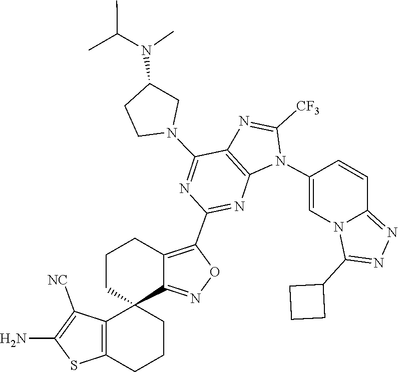 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C4CCC4)n3c2)C1] | 1.61 | 783 | B |
| II-58 | 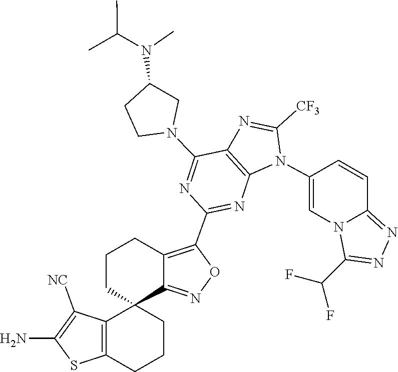 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C(F)F)n3c2)C1] | 1.60 | 779 | B |
| II-59 | 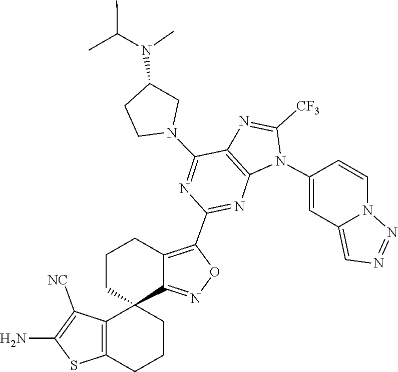 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccn3nncc3c2)C1] | 1.62 | 729 | B |
| II-60 | 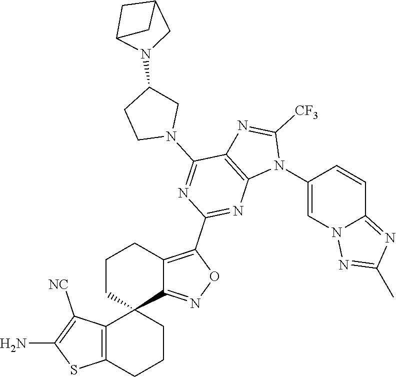 [SMILES: Cc1nc2ccc(-n3c(C(F)(F)F)nc4c(N5CC[C@H](N6CC7CC6C7)C5)nc(-c5onc6c5CCC[C@@]65CCCc6sc(N)c(C#N)c65)nc43)cn2n1] | 1.55 | 753 | B |
| II-61 | 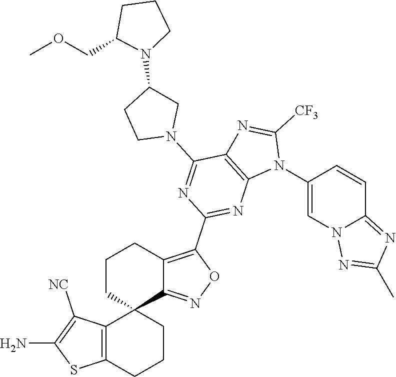 [SMILES: COC[C@@H]1CCCN1[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1] | 1.59 | 785 | B |
| II-62 | 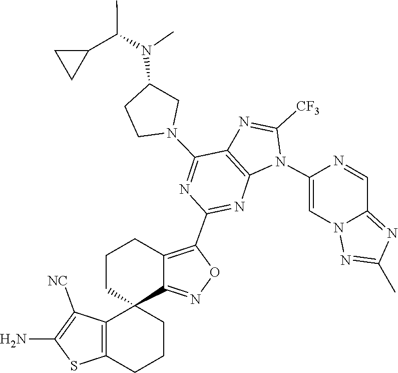 [SMILES: Cc1nc2cnc(-n3c(C(F)(F)F)nc4c(N5CC[C@H](N(C)[C@@H](C)C6CC6)C5)nc(-c5onc6c5CCC[C@@]65CCCc6sc(N)c(C#N)c65)nc43)cn2n1] | 1.74 | 770 | B |
| II-63 | 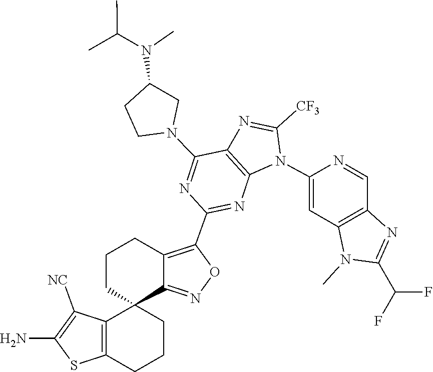 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cc3c(cn2)nc(C(F)F)n3C)C1] | 1.66 | 793 | B |
| II-64 | 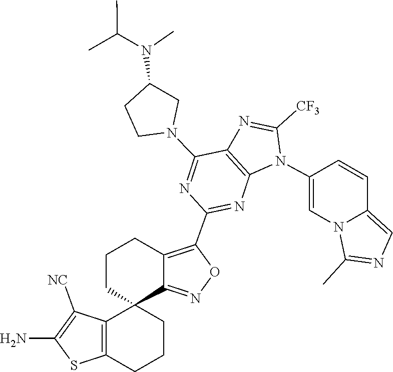 [SMILES: Cc1ncc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.65 | 742 | B |
| II-65 | 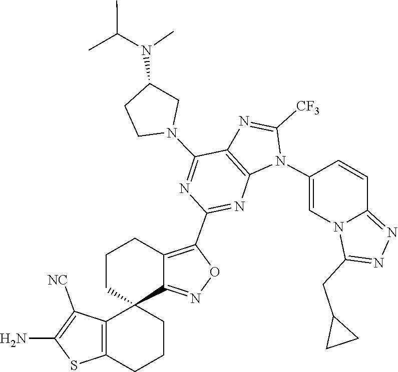 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CC4)n3c2)C1] | 1.61 | 783 | B |
| II-66 | 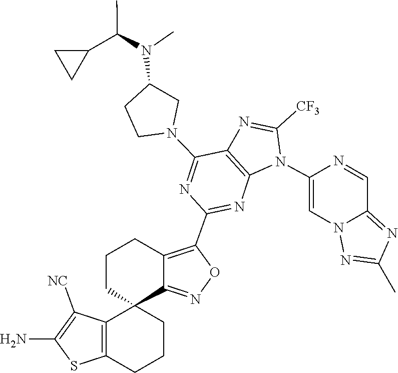 [SMILES: Cc1nc2cnc(-n3c(C(F)(F)F)nc4c(N5CC[C@H](N(C)[C@H](C)C6CC6)C5)nc(-c5onc6c5CCC[C@@]65CCCc6sc(N)c(C#N)c65)nc43)cn2n1] | 1.74 | 770 | B |
| II-67 | 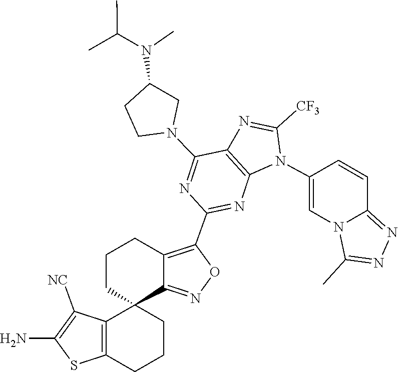 [SMILES: Cc1nnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.53 | 743 | B |
| II-68 | 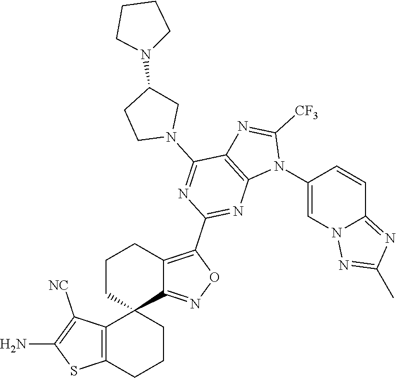 [SMILES: Cc1nc2ccc(-n3c(C(F)(F)F)nc4c(N5CC[C@H](N6CCCC6)C5)nc(-c5onc6c5CCC[C@@]65CCCc6sc(N)c(C#N)c65)nc43)cn2n1] | 1.58 | 741 | B |
| II-69 | 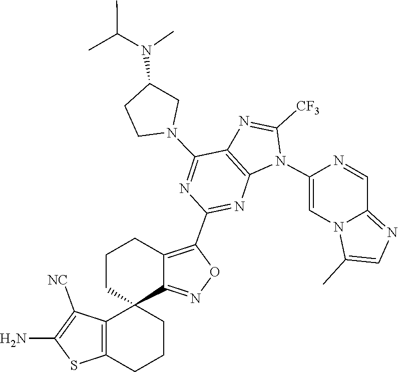 [SMILES: Cc1cnc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.62 | 743 | B |
| II-70 | 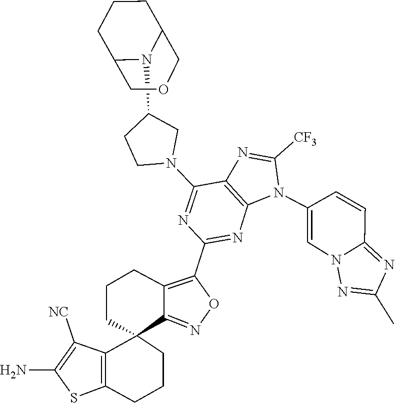 [SMILES: Cc1nc2ccc(-n3c(C(F)(F)F)nc4c(N5CC[C@H](N6C7CCCC6COC7)C5)nc(-c5onc6c5CCC[C@@]65CCCc6sc(N)c(C#N)c65)nc43)cn2n1] | 1.50 | 783 | B |
| II-71 | 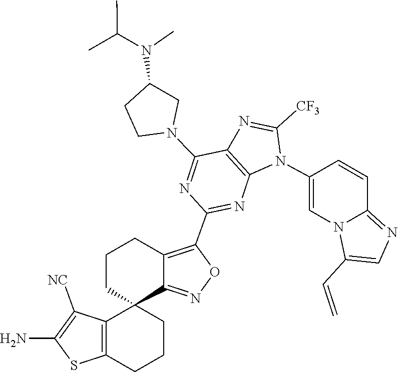 [SMILES: C=Cc1cnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.66 | 754 | B |
| II-72 | 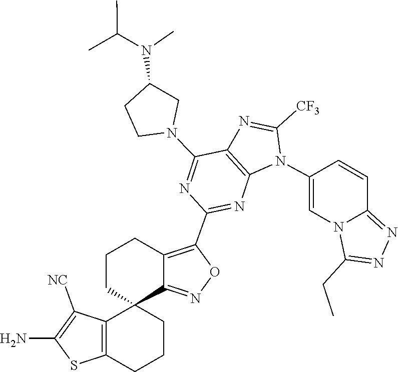 [SMILES: CCc1nnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.48 | 743 | B |
| II-73 | 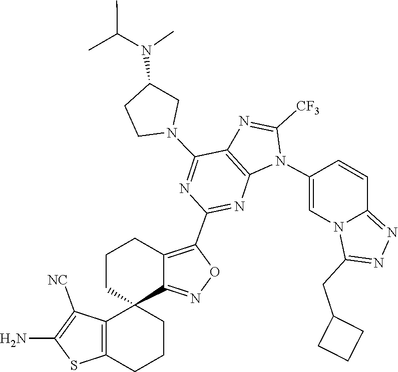 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CC4CCC4)n3c2)C1] | 1.66 | 797 | B |
| II-74 | 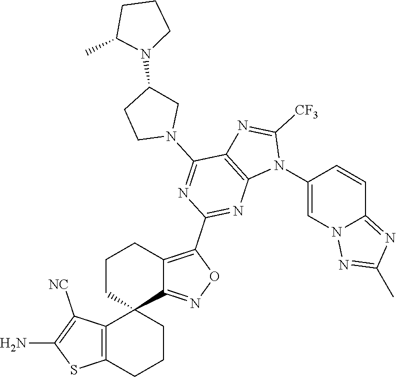 [SMILES: Cc1nc2ccc(-n3c(C(F)(F)F)nc4c(N5CC[C@H](N6CCC[C@H]6C)C5)nc(-c5onc6c5CCC[C@@]65CCCc6sc(N)c(C#N)c65)nc43)cn2n1] | 1.60 | 755 | B |
| II-75 | 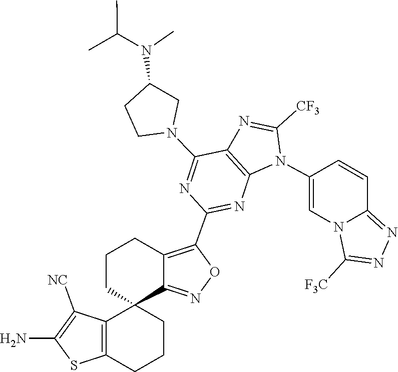 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] | 1.66 | 797 | B |
| II-76 | 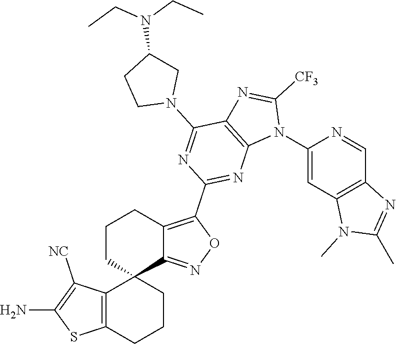 [SMILES: CCN(CC)[C@H]1CCN(c2nc(-c3onc4c3CCC[C@@]43CCCc4sc(N)c(C#N)c43)nc3c2nc(C(F)(F)F)n3-c2cc3c(cn2)nc(C)n3C)C1] | 1.57 | 757 | B |
| II-77 | 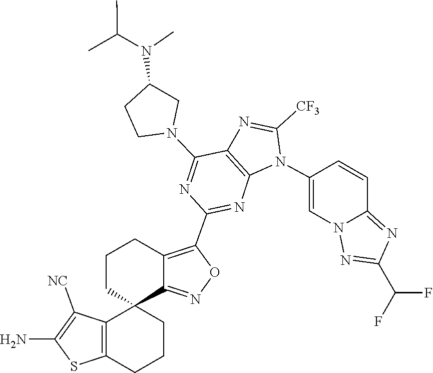 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nc(C(F)F)nn3c2)C1] | 1.64 | 779 | B |
| II-78 | 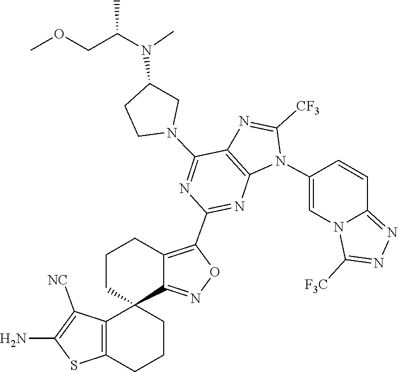 [SMILES: COC[C@H](C)N(C)[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] | 1.63 | 827 | B |
| II-79 | 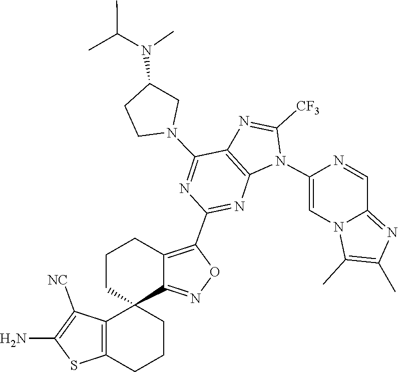 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1C] | 1.63 | 757 | B |
| II-80 | 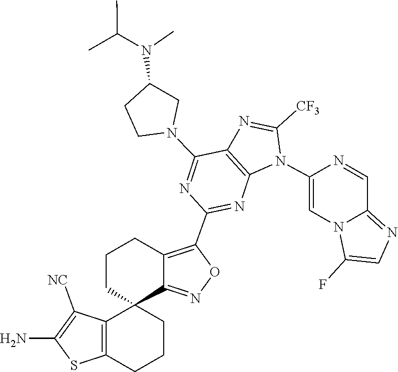 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] | 1.63 | 747 | B |
| II-81 | 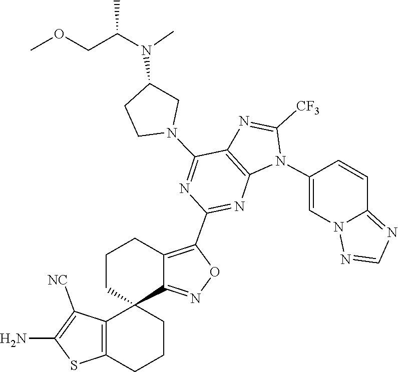 [SMILES: COC[C@H](C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1] | 1.59 | 759 | B |
| II-82 | 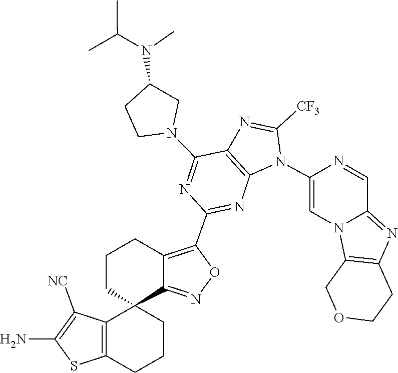 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3c4c(nc3cn2)CCOC4)C1] | 1.60 | 785 | B |
| II-83 | 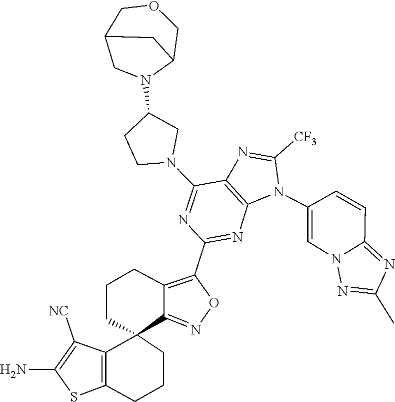 [SMILES: Cc1nc2ccc(-n3c(C(F)(F)F)nc4c(N5CC[C@H](N6CC7COCC6C7)C5)nc(-c5onc6c5CCC[C@@]65CCCc6sc(N)c(C#N)c65)nc43)cn2n1] | 1.50 | 783 | B |
| II-84 | 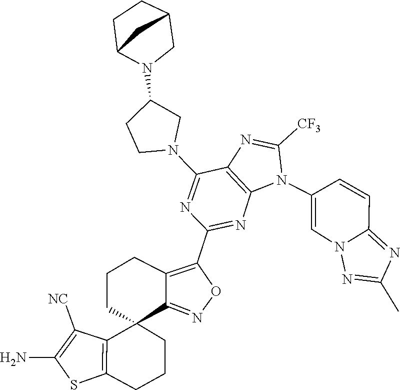 [SMILES: Cc1nc2ccc(-n3c(C(F)(F)F)nc4c(N5CC[C@H](N6C[C@H]7CC[C@@H]6C7)C5)nc(-c5onc6c5CCC[C@@]65CCCc6sc(N)c(C#N)c65)nc43)cn2n1] | 1.65 | 767 | B |
| II-85 | 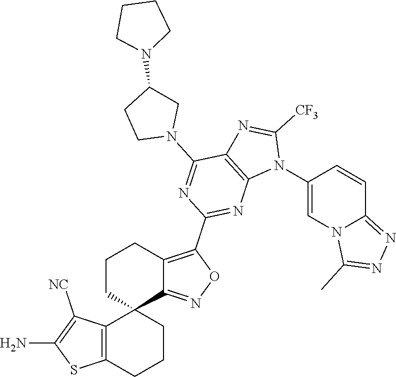 [SMILES: Cc1nnc2ccc(-n3c(C(F)(F)F)nc4c(N5CC[C@H](N6CCCC6)C5)nc(-c5onc6c5CCC[C@@]65CCCc6sc(N)c(C#N)c65)nc43)cn12] | 1.49 | 741 | B |
| II-86 | 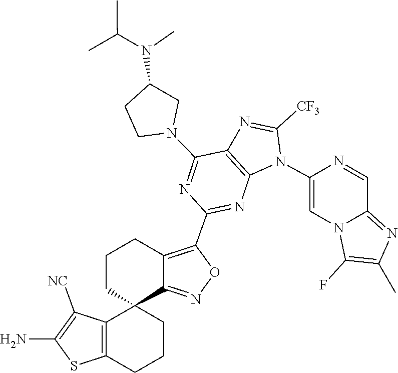 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1F] | 1.69 | 761 | B |
| II-87 | 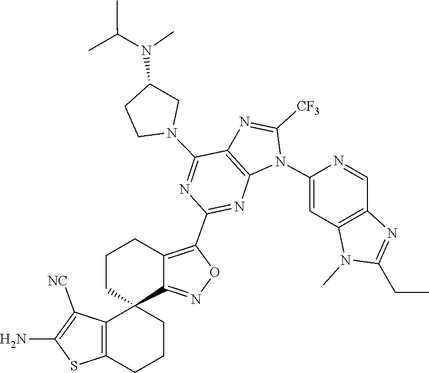 [SMILES: CCc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] | 1.62 | 771 | B |
| II-88 | 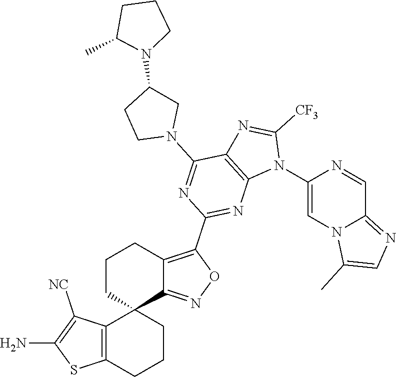 [SMILES: Cc1cnc2cnc(-n3c(C(F)(F)F)nc4c(N5CC[C@H](N6CCC[C@H]6C)C5)nc(-c5onc6c5CCC[C@@]65CCCc6sc(N)c(C#N)c65)nc43)cn12] | 1.61 | 755 | B |
| II-89 | 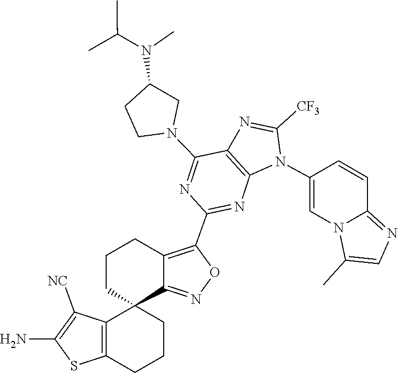 [SMILES: Cc1cnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.63 | 742 | B |
| II-90 | 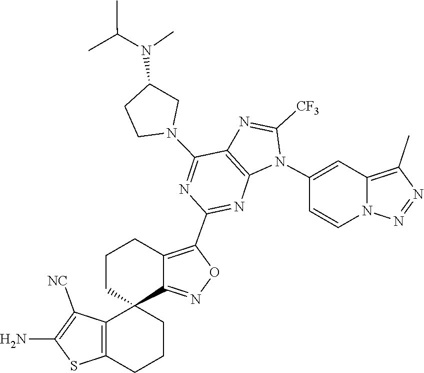 [SMILES: Cc1nnn2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cc12] | 1.64 | 743 | B |
| II-91 | 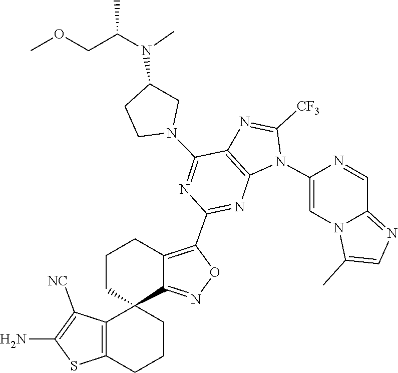 [SMILES: COC[C@H](C)N(C)[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] | 1.61 | 773 | B |
| II-92 | 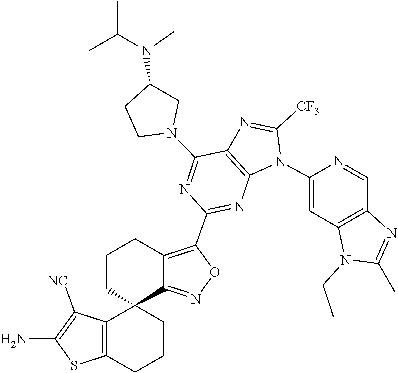 [SMILES: CCn1c(C)nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc21] | 1.61 | 771 | B |
| II-93 | 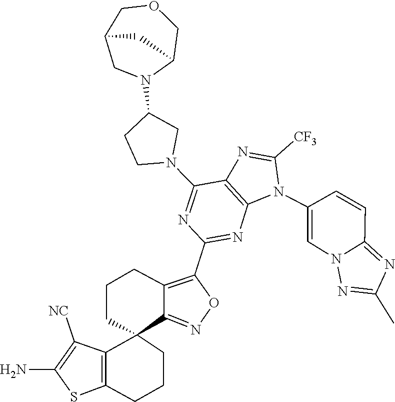 [SMILES: Cc1nc2ccc(-n3c(C(F)(F)F)nc4c(N5CC[C@H](N6C[C@H]7COC[C@@H]6C7)C5)nc(-c5onc6c5CCC[C@@]65CCCc6sc(N)c(C#N)c65)nc43)cn2n1] | 1.49 | 783 | B |
| II-94 | 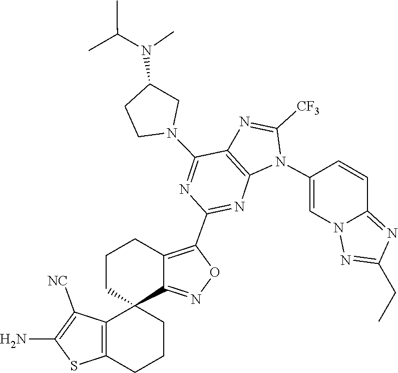 [SMILES: CCc1nc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1] | 1.66 | 757 | B |
| II-95 | 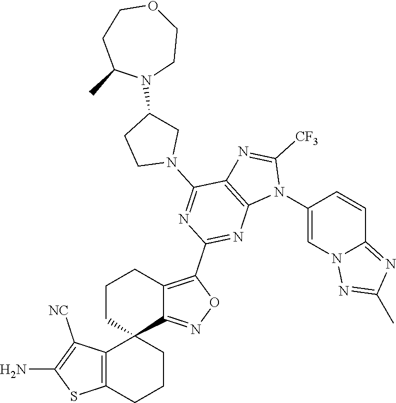 [SMILES: Cc1nc2ccc(-n3c(C(F)(F)F)nc4c(N5CC[C@H](N6CCOCC[C@@H]6C)C5)nc(-c5onc6c5CCC[C@@]65CCCc6sc(N)c(C#N)c65)nc43)cn2n1] | 1.59 | 785 | B |
| II-96 | 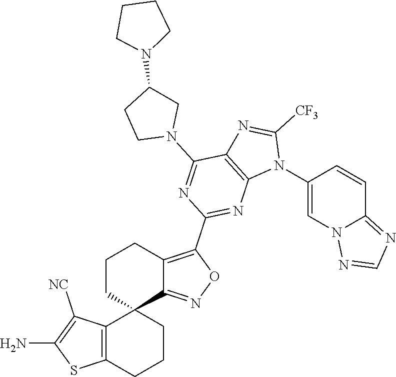 [SMILES: N#Cc1c(N)sc2c1[C@@]1(CCC2)CCCc2c1noc2-c1nc(N2CC[C@H](N3CCCC3)C2)c2nc(C(F)(F)F)n(-c3ccc4ncnn4c3)c2n1] | 1.57 | 727 | B |
| II-97 | 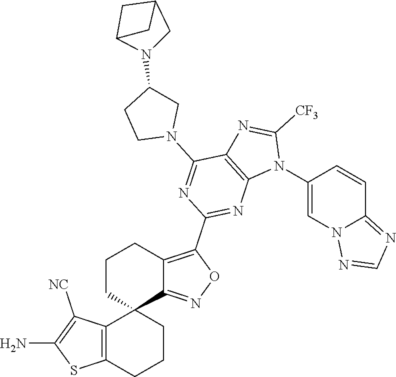 [SMILES: N#Cc1c(N)sc2c1[C@@]1(CCC2)CCCc2c1noc2-c1nc(N2CC[C@H](N3CC4CC3C4)C2)c2nc(C(F)(F)F)n(-c3ccc4ncnn4c3)c2n1] | 1.53 | 793 | B |
| II-98 | 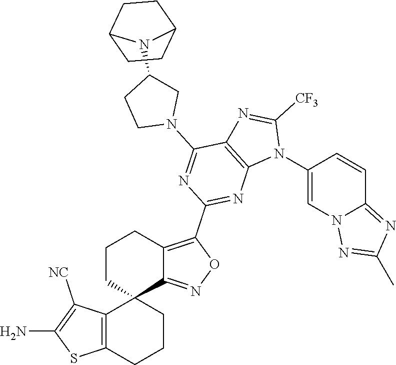 [SMILES: Cc1nc2ccc(-n3c(C(F)(F)F)nc4c(N5CC[C@H](N6C7CCC6CC7)C5)nc(-c5onc6c5CCC[C@@]65CCCc6sc(N)c(C#N)c65)nc43)cn2n1] | 1.64 | 384 (dictation) | B |
| II-99 | 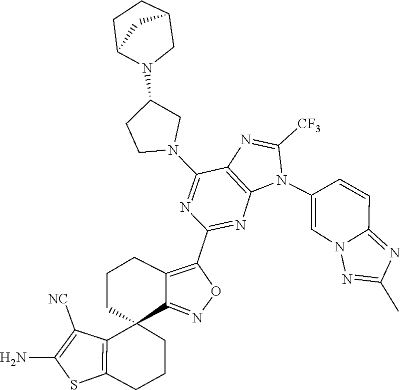 [SMILES: Cc1nc2ccc(-n3c(C(F)(F)F)nc4c(N5CC[C@H](N6C[C@@H]7CC[C@H]6C7)C5)nc(-c5onc6c5CCC[C@@]65CCCc6sc(N)c(C#N)c65)nc43)cn2n1] | 1.65 | 767 | B |
| II-100 | 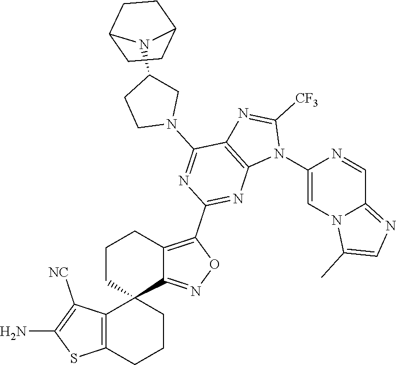 [SMILES: Cc1cnc2cnc(-n3c(C(F)(F)F)nc4c(N5CC[C@H](N6C7CCC6CC7)C5)nc(-c5onc6c5CCC[C@@]65CCCc6sc(N)c(C#N)c65)nc43)cn12] | 1.54 | 767 | B |
| II-101 | 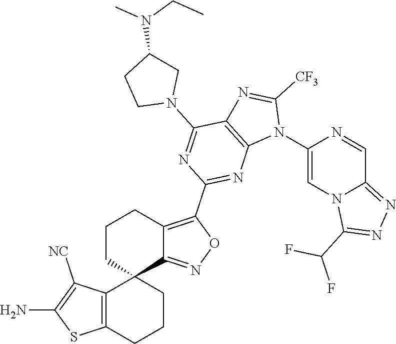 [SMILES: CCN(C)[C@H]1CCN(c2nc(-c3onc4c3CCC[C@@]43CCCc4sc(N)c(C#N)c43)nc3c2nc(C(F)(F)F)n3-c2cn3c(C(F)F)nnc3cn2)C1] | 1.53 | 765 | B |
| II-102 | 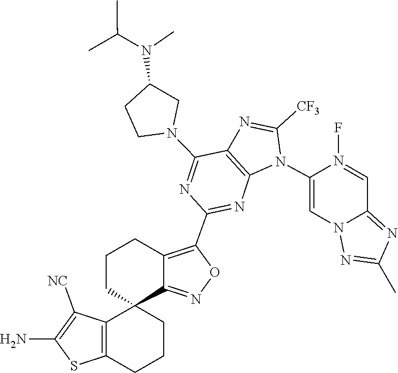 | 1.56 | 761 | B |
| II-103 | 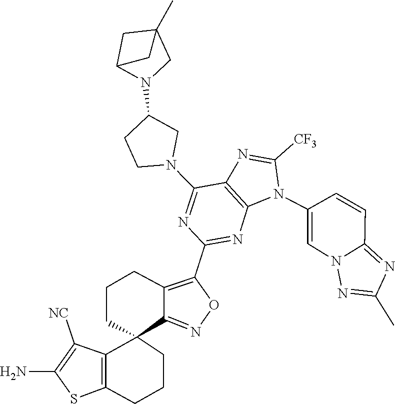 [SMILES: Cc1nc2ccc(-n3c(C(F)(F)F)nc4c(N5CC[C@H](N6CC7(C)CC6C7)C5)nc(-c5onc6c5CCC[C@@]65CCCc6sc(N)c(C#N)c65)nc43)cn2n1] | 1.64 | 767 | B |
| II-104 | 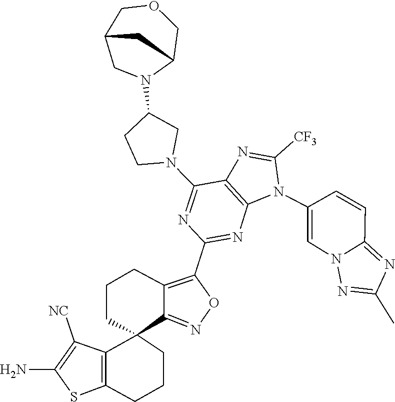 [SMILES: Cc1nc2ccc(-n3c(C(F)(F)F)nc4c(N5CC[C@H](N6C[C@@H]7COC[C@H]6C7)C5)nc(-c5onc6c5CCC[C@@]65CCCc6sc(N)c(C#N)c65)nc43)cn2n1] | 1.49 | 783 | B |
| II-105 | 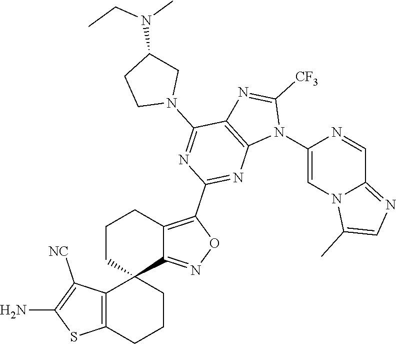 [SMILES: CCN(C)[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] | 1.53 | 729 | B |
| II-106 | 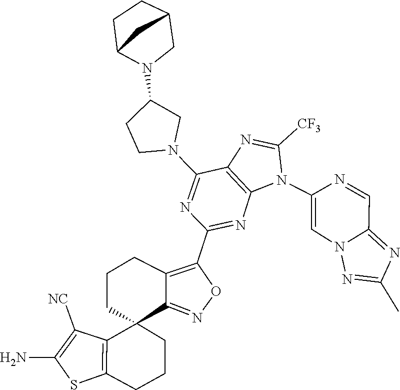 [SMILES: Cc1nc2cnc(-n3c(C(F)(F)F)nc4c(N5CC[C@H](N6C[C@H]7CC[C@@H]6C7)C5)nc(-c5onc6c5CCC[C@@]65CCCc6sc(N)c(C#N)c65)nc43)cn2n1] | 1.69 | 768 | B |
| II-107 | 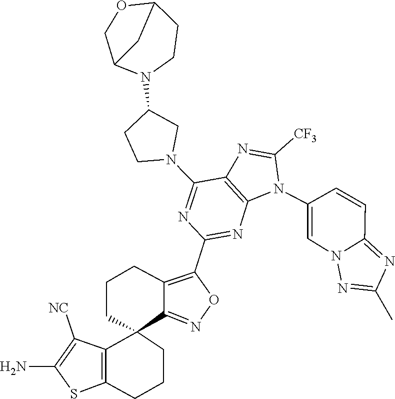 [SMILES: Cc1nc2ccc(-n3c(C(F)(F)F)nc4c(N5CC[C@H](N6CCC7CC6CO7)C5)nc(-c5onc6c5CCC[C@@]65CCCc6sc(N)c(C#N)c65)nc43)cn2n1] | 1.44 | 783 | B |
| II-108 | 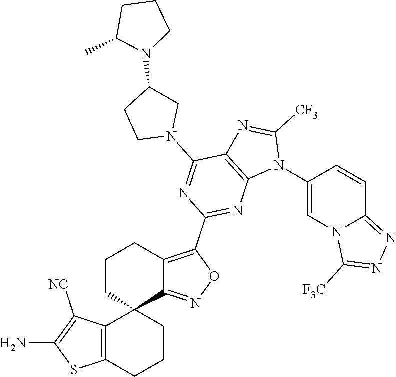 [SMILES: C[C@@H]1CCCN1[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] | 1.67 | 809 | B |
| II-109 | 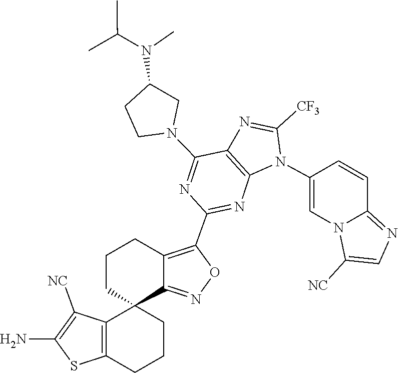 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncc(C#N)n3c2)C1] | 1.64 | 754 | B |
| II-110 | 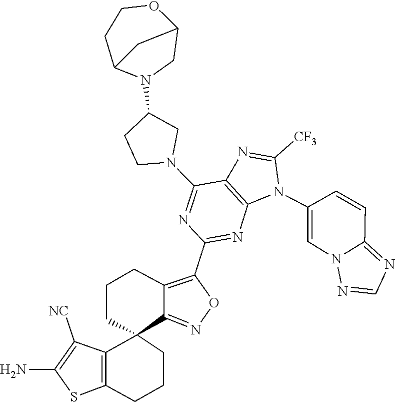 [SMILES: N#Cc1c(N)sc2c1[C@@]1(CCC2)CCCc2c1noc2-c1nc(N2CC[C@H](N3CC4CC3CCO4)C2)c2nc(C(F)(F)F)n(-c3ccc4ncnn4c3)c2n1] | 1.47 | 769 | B |
| II-111 | 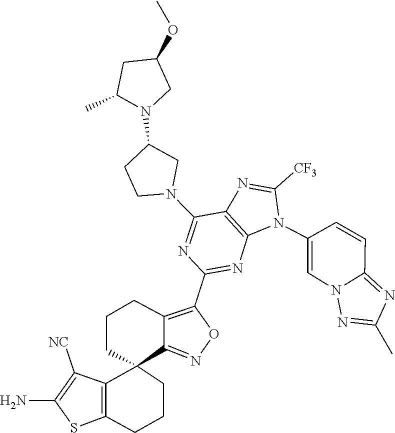 [SMILES: CO[C@@H]1C[C@@H](C)N([C@H]2CCN(c3nc(-c4onc5c4CCC[C@@]54CCCc5sc(N)c(C#N)c54)nc4c3nc(C(F)(F)F)n4-c3ccc4nc(C)nn4c3)C2)C1] | 1.55 | 785 | B |
| II-112 | 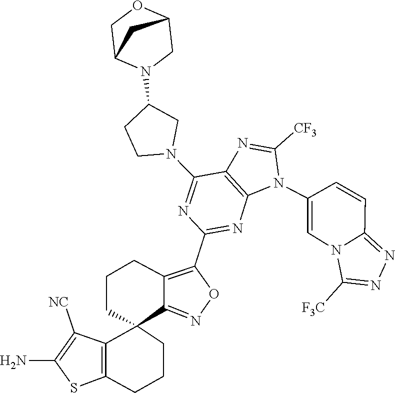 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3ccc4nnc(C(F)(F)F)n4c3)c2n1] | 1.48 | 823 | B |
| II-113 | 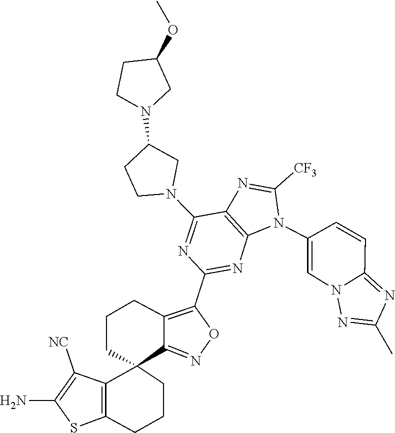 [SMILES: CO[C@@H]1CCN([C@H]2CCN(c3nc(-c4onc5c4CCC[C@@]54CCCc5sc(N)c(C#N)c54)nc4c3nc(C(F)(F)F)n4-c3ccc4nc(C)nn4c3)C2)C1] | 1.49 | 771 | B |
| II-114 | 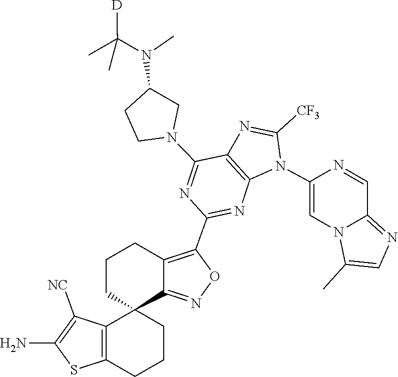 [SMILES: [2H]C(C)(C)N(C)[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] | 1.61 | 744 | B |
| II-115 | 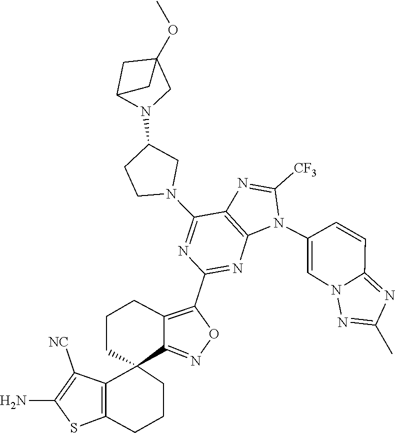 [SMILES: COC12CC(C1)N([C@H]1CCN(c3nc(-c4onc5c4CCC[C@@]54CCCc5sc(N)c(C#N)c54)nc4c3nc(C(F)(F)F)n4-c3ccc4nc(C)nn4c3)C1)C2] | 1.50 | 783 | B |
| II-116 | 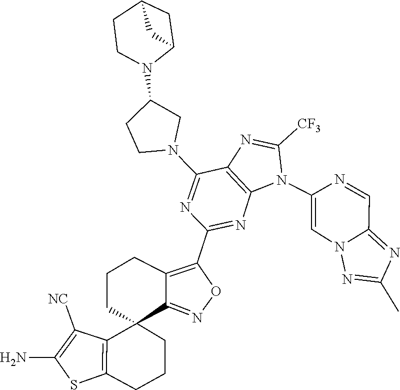 [SMILES: Cc1nc2cnc(-n3c(C(F)(F)F)nc4c(N5CC[C@H](N6CC[C@H]7C[C@@H]6C7)C5)nc(-c5onc6c5CCC[C@@]65CCCc6sc(N)c(C#N)c65)nc43)cn2n1] | 1.69 | 768 | B |
| II-117 | 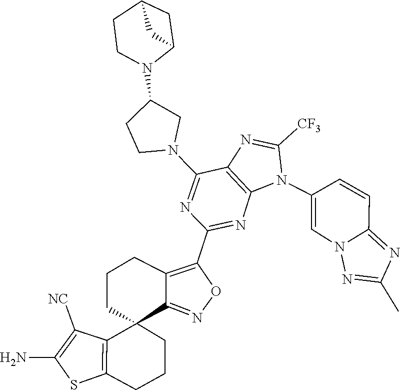 [SMILES: Cc1nc2ccc(-n3c(C(F)(F)F)nc4c(N5CC[C@H](N6CC[C@H]7C[C@@H]6C7)C5)nc(-c5onc6c5CCC[C@@]65CCCc6sc(N)c(C#N)c65)nc43)cn2n1] | 1.65 | 767 | B |
| II-118 | 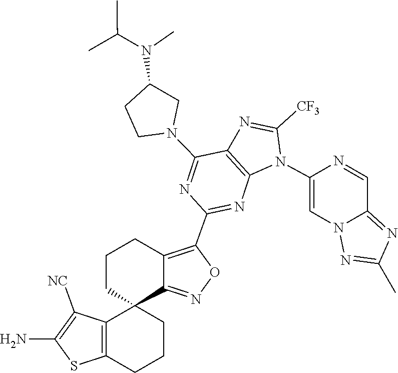 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1] | 1.65 | 744 | B |
| II-119 | 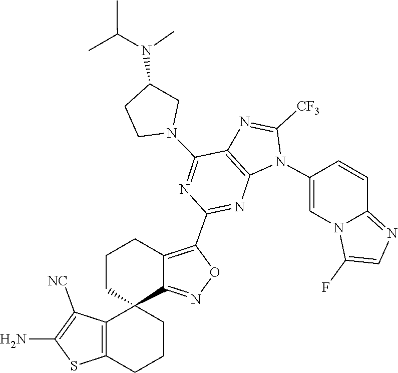 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncc(F)n3c2)C1] | 1.65 | 746 | B |
| II-120 | 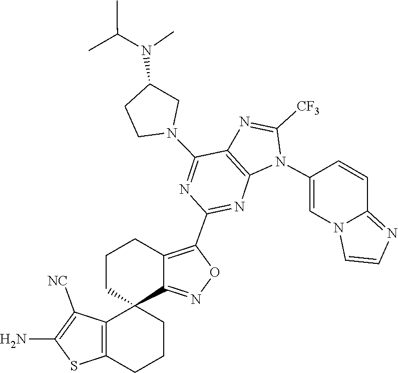 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nccn3c2)C1] | 1.57 | 729 | B |
| II-121 | 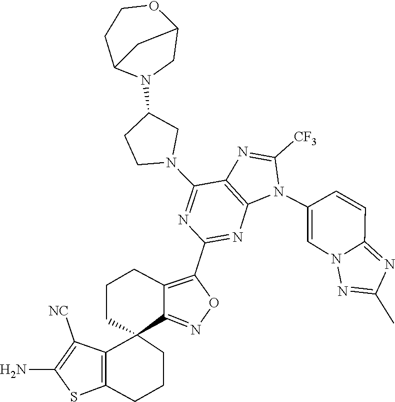 [SMILES: Cc1nc2ccc(-n3c(C(F)(F)F)nc4c(N5CC[C@H](N6CC7CC6CCO7)C5)nc(-c5onc6c5CCC[C@@]65CCCc6sc(N)c(C#N)c65)nc43)cn2n1] | 1.50 | 783 | B |
| II-122 | 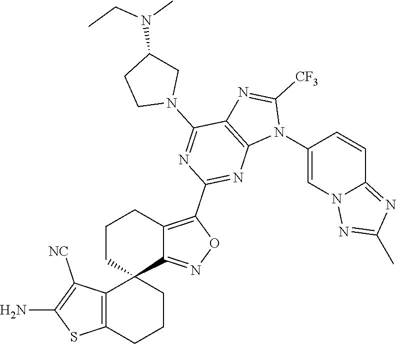 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1] | 1.50 | 729 | B |
| II-123 | 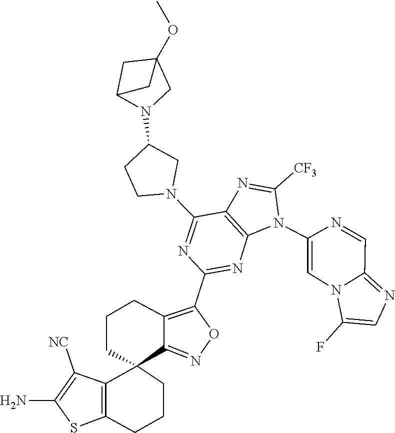 [SMILES: COC12CC(C1)N([C@H]1CCN(c3nc(-c4onc5c4CCC[C@@]54CCCc5sc(N)c(C#N)c54)nc4c3nc(C(F)(F)F)n4-c3cn4c(F)cnc4cn3)C1)C2] | 1.51 | 787 | B |
| II-124 | 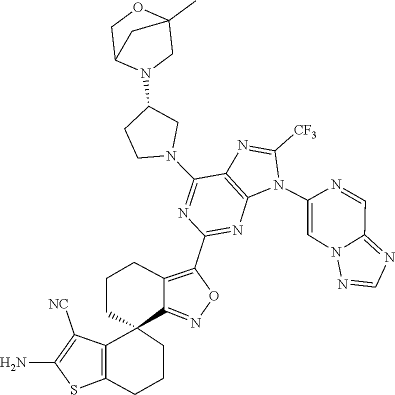 [SMILES: CC12CC(CO1)N([C@H]1CCN(c3nc(-c4onc5c4CCC[C@@]54CCCc5sc(N)c(C#N)c54)nc4c3nc(C(F)(F)F)n4-c3cn4ncnc4cn3)C1)C2] | 1.45 | 769 | B |
| II-125 | 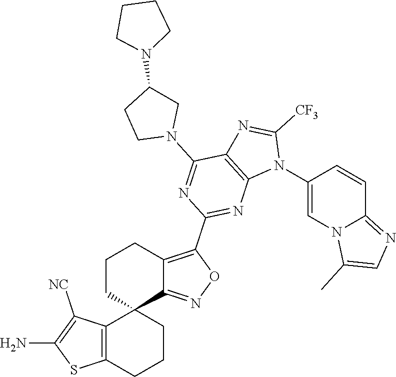 [SMILES: Cc1cnc2ccc(-n3c(C(F)(F)F)nc4c(N5CC[C@H](N6CCCC6)C5)nc(-c5onc6c5CCC[C@@]65CCCc6sc(N)c(C#N)c65)nc43)cn12] | 1.58 | 740 | B |
| II-126 | 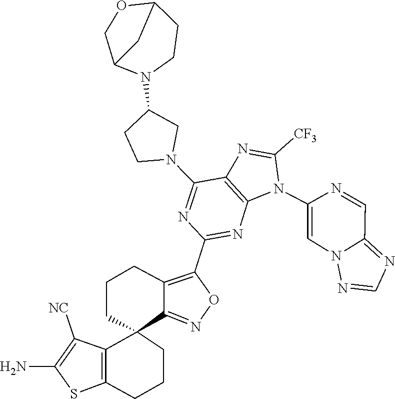 [SMILES: N#Cc1c(N)sc2c1[C@@]1(CCC2)CCCc2c1noc2-c1nc(N2CC[C@H](N3CCC4CC3CO4)C2)c2nc(C(F)(F)F)n(-c3cn4ncnc4cn3)c2n1] | 1.40 | 769 | B |
| II-127 | 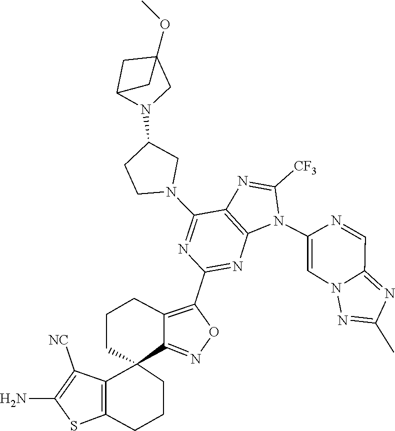 [SMILES: COC12CC(C1)N([C@H]1CCN(c3nc(-c4onc5c4CCC[C@@]54CCCc5sc(N)c(C#N)c54)nc4c3nc(C(F)(F)F)n4-c3cn4nc(C)nc4cn3)C1)C2] | 1.65 | 812p | B |
| II-128 | 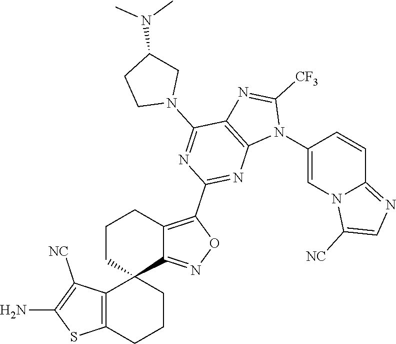 [SMILES: CN(C)[C@H]1CCN(c2nc(-c3onc4c3CCC[C@@]43CCCc4sc(N)c(C#N)c43)nc3c2nc(C(F)(F)F)n3-c2ccc3ncc(C#N)n3c2)C1] | 1.45 | 725 | B |
| II-129 | 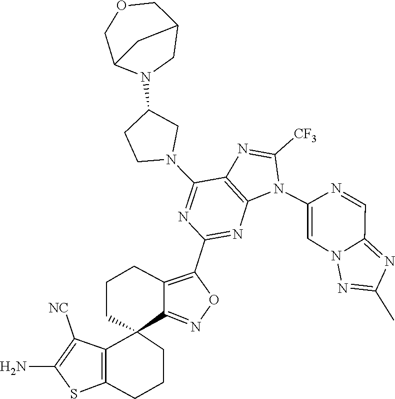 [SMILES: Cc1nc2cnc(-n3c(C(F)(F)F)nc4c(N5CC[C@H](N6CC7COCC6C7)C5)nc(-c5onc6c5CCC[C@@]65CCCc6sc(N)c(C#N)c65)nc43)cn2n1] | 1.52 | 784 | B |
| II-130 | 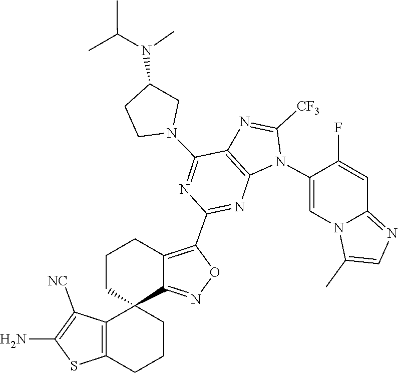 [SMILES: Cc1cnc2cc(F)c(-n3c(C(F)(F)F)nc4c(N5CC[C@H](N(C)C(C)C)C5)nc(-c5onc6c5CCC[C@@]65CCCc6sc(N)c(C#N)c65)nc43)cn12] | 1.66 | 760 | B |
| II-131 | 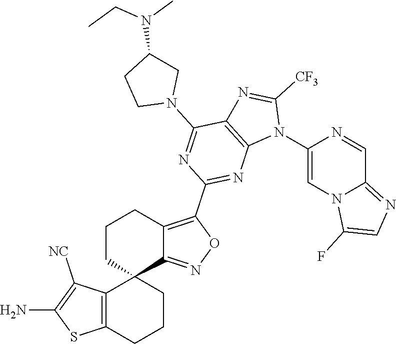 [SMILES: CCN(C)[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] | 1.57 | 733 | B |
| II-132 | 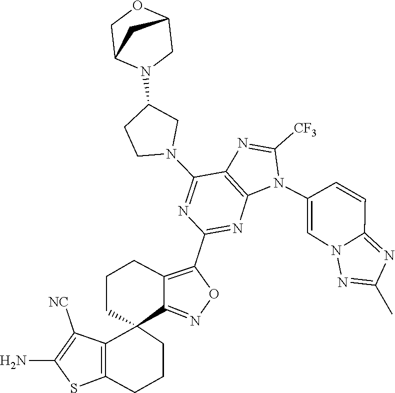 [SMILES: Cc1nc2ccc(-n3c(C(F)(F)F)nc4c(N5CC[C@H](N6C[C@@H]7C[C@H]6CO7)C5)nc(-c5onc6c5CCC[C@@]65CCCc6sc(N)c(C#N)c65)nc43)cn2n1] | 1.41 | 769 | B |
| II-133 | 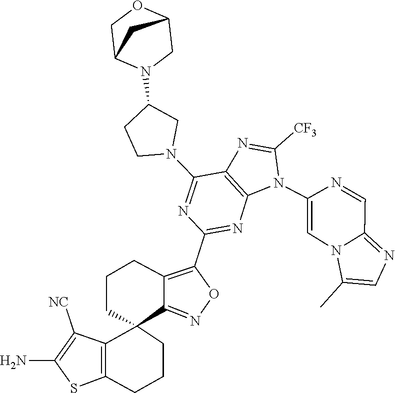 [SMILES: Cc1cnc2cnc(-n3c(C(F)(F)F)nc4c(N5CC[C@H](N6C[C@@H]7C[C@H]6CO7)C5)nc(-c5onc6c5CCC[C@@]65CCCc6sc(N)c(C#N)c65)nc43)cn12] | 1.39 | 769 | B |
| II-134 | 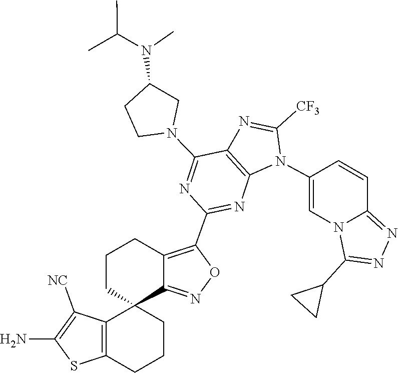 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C4CC4)n3c2)C1] | 1.69 | 769 | B |
| II-135 | 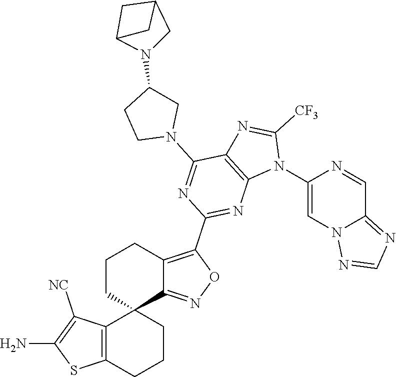 [SMILES: N#Cc1c(N)sc2c1[C@@]1(CCC2)CCCc2c1noc2-c1nc(N2CC[C@H](N3CC4CC3C4)C2)c2nc(C(F)(F)F)n(-c3cn4ncnc4cn3)c2n1] | 1.56 | 740 | B |
| II-136 | 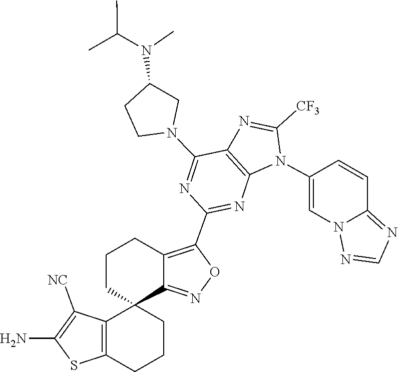 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1] | 1.51 | 729 | B |
| II-137 | 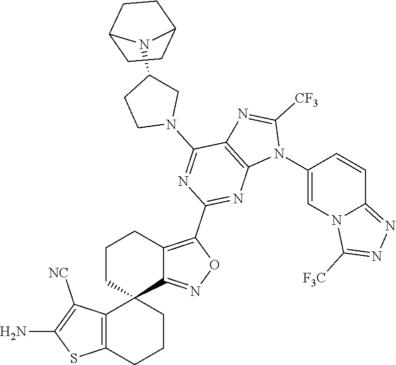 [SMILES: N#Cc1c(N)sc2c1[C@@]1(CCC2)CCCc2c1noc2-c1nc(N2CC[C@H](N3C4CCC3CC4)C2)c2nc(C(F)(F)F)n(-c3ccc4nnc(C(F)(F)F)n4c3)c2n1] | 1.68 | 821 | B |
| II-138 | 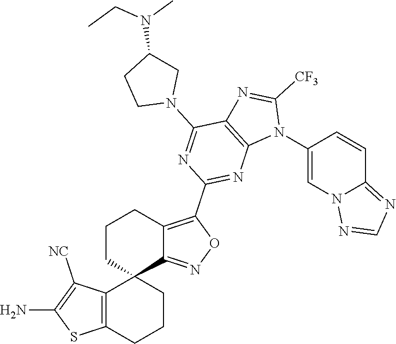 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1] | 1.51 | 715 | B |
| II-139 | 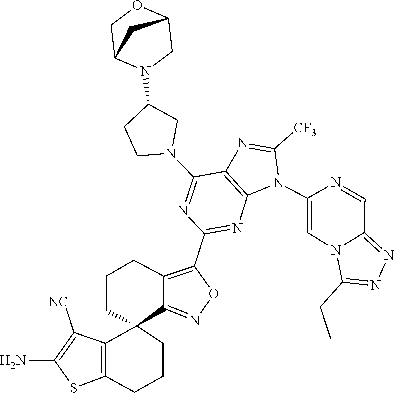 [SMILES: CCc1nnc2cnc(-n3c(C(F)(F)F)nc4c(N5CC[C@H](N6C[C@@H]7C[C@H]6CO7)C5)nc(-c5onc6c5CCC[C@@]65CCCc6sc(N)c(C#N)c65)nc43)cn12] | 1.38 | 784 | B |
| II-140 | 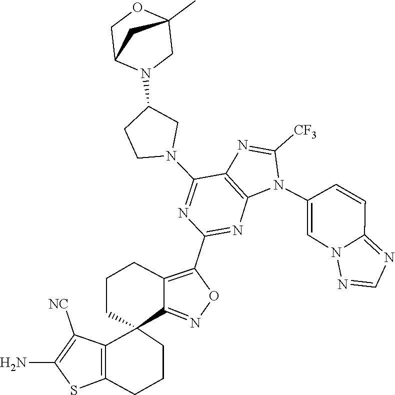 [SMILES: C[C@@]12C[C@@H](CO1)N([C@H]1CCN(c3nc(-c4onc5c4CCC[C@@]54CCCc5sc(N)c(C#N)c54)nc4c3nc(C(F)(F)F)n4-c3ccc4ncnn4c3)C1)C2] | 1.45 | 769 | B |
| II-141 | 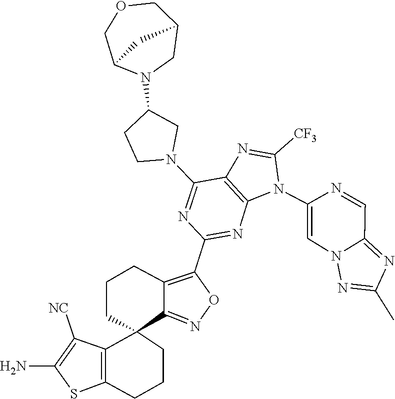 [SMILES: Cc1nc2cnc(-n3c(C(F)(F)F)nc4c(N5CC[C@H](N6C[C@@H]7COC[C@H]6C7)C5)nc(-c5onc6c5CCC[C@@]65CCCc6sc(N)c(C#N)c65)nc43)cn2n1] | 1.53 | 784 | B |
| II-142 | 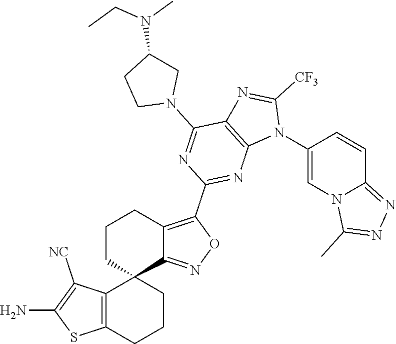 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3nnc(C)n3c2)C1] | 1.45 | 729 | B |
| II-143 | 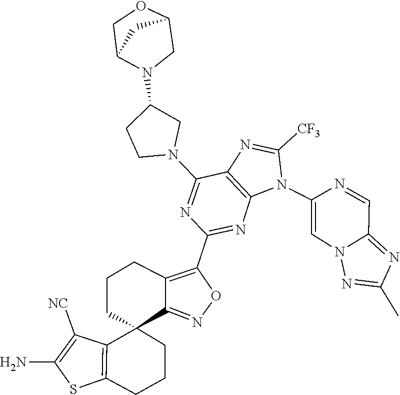 [SMILES: Cc1nc2cnc(-n3c(C(F)(F)F)nc4c(N5CC[C@H](N6C[C@H]7C[C@@H]6CO7)C5)nc(-c5onc6c5CCC[C@@]65CCCc6sc(N)c(C#N)c65)nc43)cn2n1] | 1.41 | 769 | B |
| II-144 | 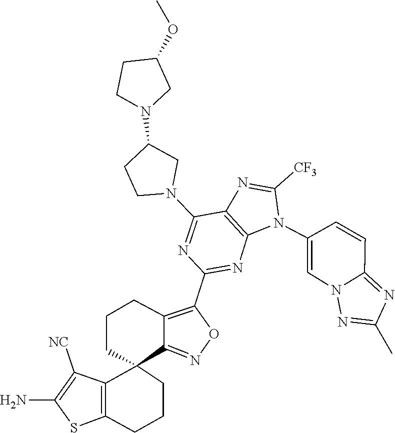 [SMILES: CO[C@H]1CCN([C@H]2CCN(c3nc(-c4onc5c4CCC[C@@]54CCCc5sc(N)c(C#N)c54)nc4c3nc(C(F)(F)F)n4-c3ccc4nc(C)nn4c3)C2)C1] | 1.49 | 771 | B |
| II-145 | 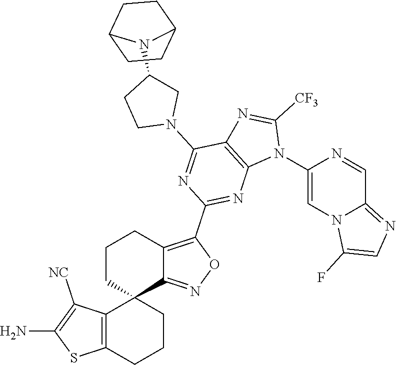 [SMILES: N#Cc1c(N)sc2c1[C@@]1(CCC2)CCCc2c1noc2-c1nc(N2CC[C@H](N3C4CCC3CC4)C2)c2nc(C(F)(F)F)n(-c3cn4c(F)cnc4cn3)c2n1] | 1.65 | 771 | B |
| II-146 | 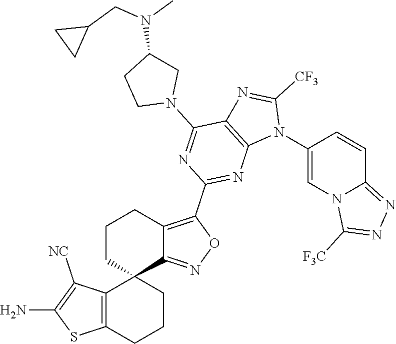 [SMILES: CN(CC1CC1)[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] | 1.66 | 809 | B |
| II-147 | 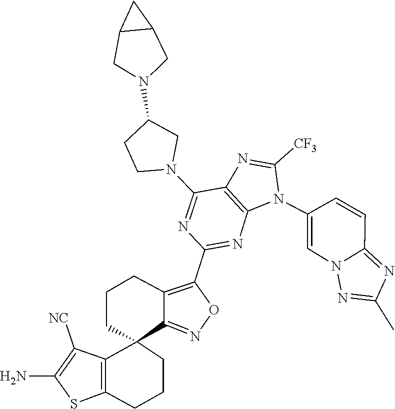 [SMILES: Cc1nc2ccc(-n3c(C(F)(F)F)nc4c(N5CC[C@H](N6CC7CC7C6)C5)nc(-c5onc6c5CCC[C@@]65CCCc6sc(N)c(C#N)c65)nc43)cn2n1] | 1.71 | 753 | B |
| II-148 | 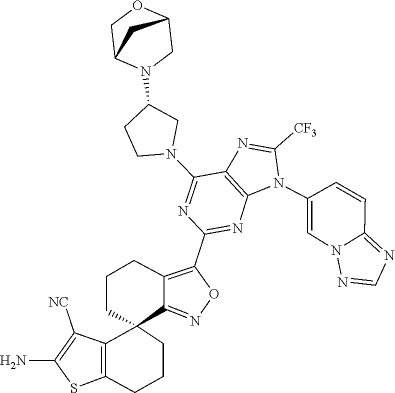 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3ccc4ncnn4c3)c2n1] | 1.38 | 755 | B |
| II-149 | 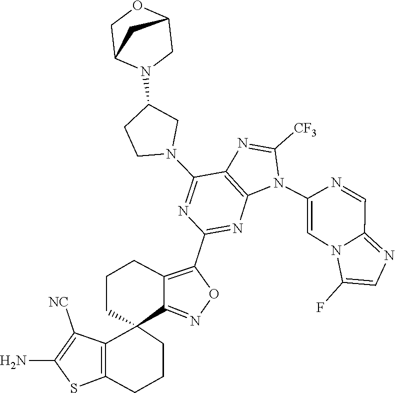 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3cn4c(F)cnc4cn3)c2n1] | 1.44 | 773 | B |
| II-150 | 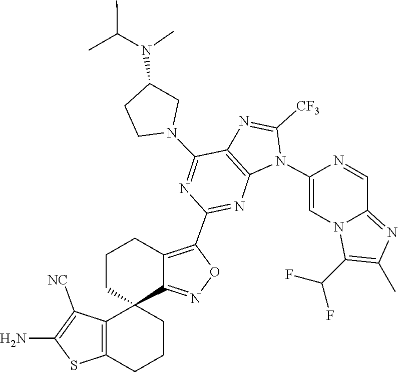 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1C(F)F] | 1.69 | 793 | B |
| II-151 | 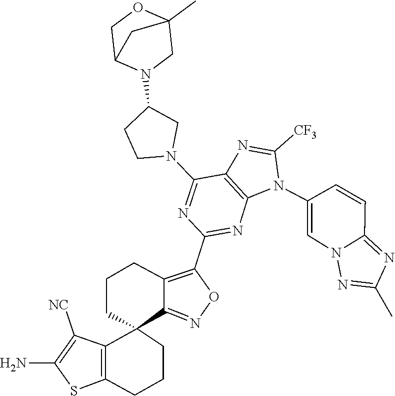 [SMILES: Cc1nc2ccc(-n3c(C(F)(F)F)nc4c(N5CC[C@H](N6CC7(C)CC6CO7)C5)nc(-c5onc6c5CCC[C@@]65CCCc6sc(N)c(C#N)c65)nc43)cn2n1] | 1.47 | 783 | B |
| II-152 | 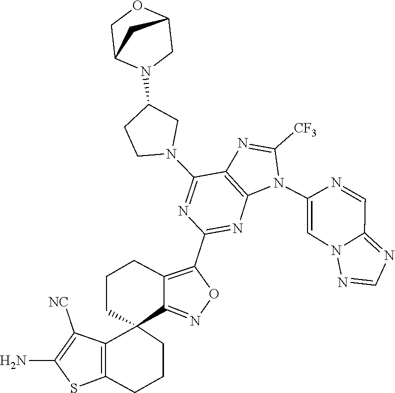 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3cn4ncnc4cn3)c2n1] | 1.41 | 756 | B |
| II-153 | 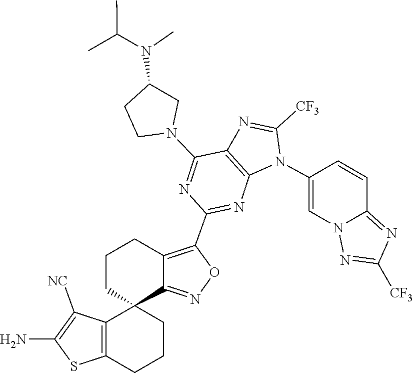 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nc(C(F)(F)F)nn3c2)C1] | 1.76 | 797 | B |
| II-154 | 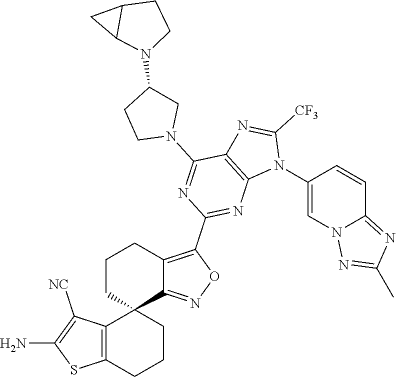 [SMILES: Cc1nc2ccc(-n3c(C(F)(F)F)nc4c(N5CC[C@H](N6CCC7CC76)C5)nc(-c5onc6c5CCC[C@@]65CCCc6sc(N)c(C#N)c65)nc43)cn2n1] | 1.58 | 753 | B |
| II-155 | 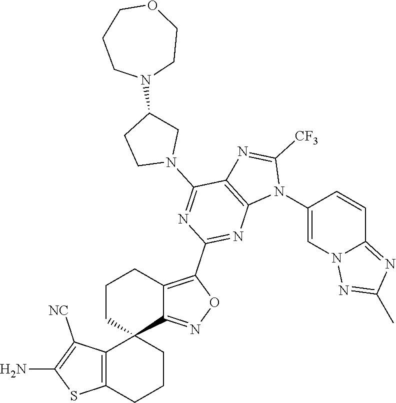 [SMILES: Cc1nc2ccc(-n3c(C(F)(F)F)nc4c(N5CC[C@H](N6CCCOCC6)C5)nc(-c5onc6c5CCC[C@@]65CCCc6sc(N)c(C#N)c65)nc43)cn2n1] | 1.47 | 771 | B |
| II-156 | 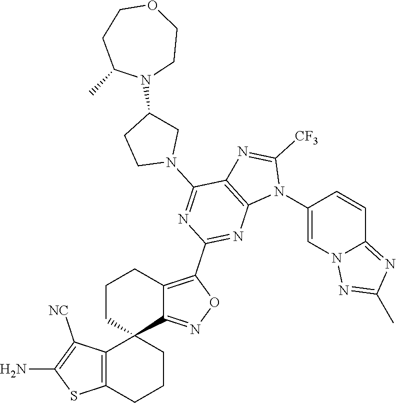 [SMILES: Cc1nc2ccc(-n3c(C(F)(F)F)nc4c(N5CC[C@H](N6CCOCC[C@H]6C)C5)nc(-c5onc6c5CCC[C@@]65CCCc6sc(N)c(C#N)c65)nc43)cn2n1] | 1.57 | 785 | B |
| II-157 | 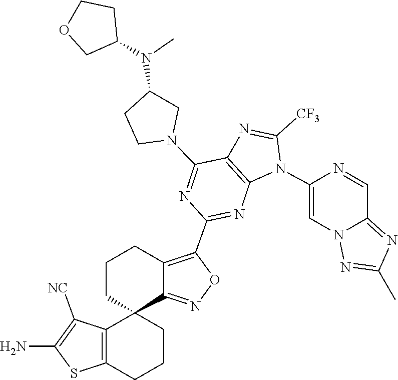 [SMILES: Cc1nc2cnc(-n3c(C(F)(F)F)nc4c(N5CC[C@H](N(C)[C@H]6CCOC6)C5)nc(-c5onc6c5CCC[C@@]65CCCc6sc(N)c(C#N)c65)nc43)cn2n1] | 1.48 | 772 | B |
| II-158 | 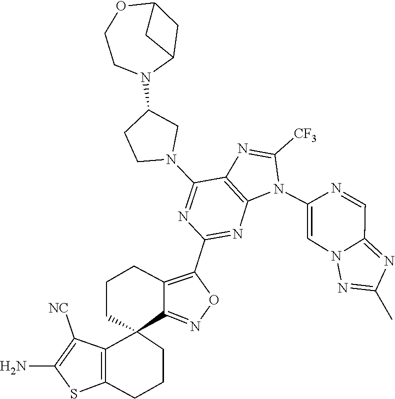 [SMILES: Cc1nc2cnc(-n3c(C(F)(F)F)nc4c(N5CC[C@H](N6CCOC7CC6C7)C5)nc(-c5onc6c5CCC[C@@]65CCCc6sc(N)c(C#N)c65)nc43)cn2n1] | 1.48 | 784 | B |
| II-159 | 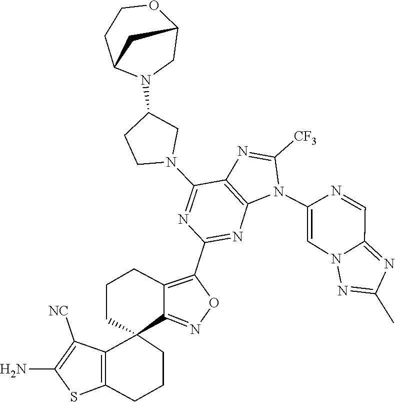 [SMILES: Cc1nc2cnc(-n3c(C(F)(F)F)nc4c(N5CC[C@H](N6C[C@@H]7C[C@H]6CCO7)C5)nc(-c5onc6c5CCC[C@@]65CCCc6sc(N)c(C#N)c65)nc43)cn2n1] | 1.53 | 784 | B |
| II-160 | 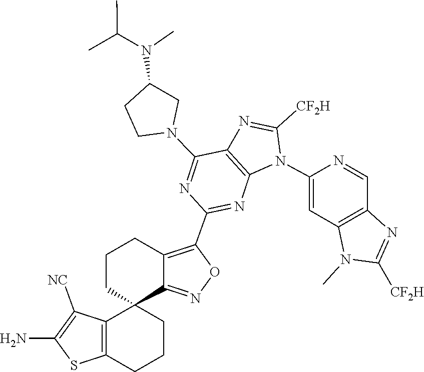 [SMILES: CC(C)N(C)[C@H]1CCN(c2nc(-c3onc4c3CCC[C@@]43CCCc4sc(N)c(C#N)c43)nc3c2nc(C(F)F)n3-c2cc3c(cn2)nc(C(F)F)n3C)C1] | 1.62 | 775 | B |
| II-161 | 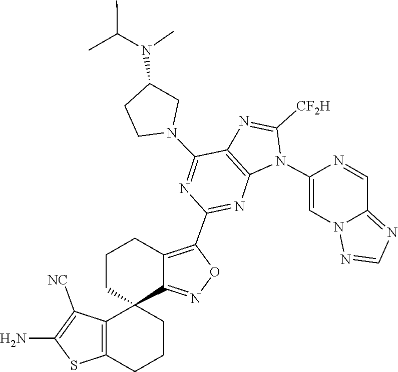 [SMILES: CC(C)N(C)[C@H]1CCN(c2nc(-c3onc4c3CCC[C@@]43CCCc4sc(N)c(C#N)c43)nc3c2nc(C(F)F)n3-c2cn3ncnc3cn2)C1] | 1.56 | 712 | B |
| II-162 | 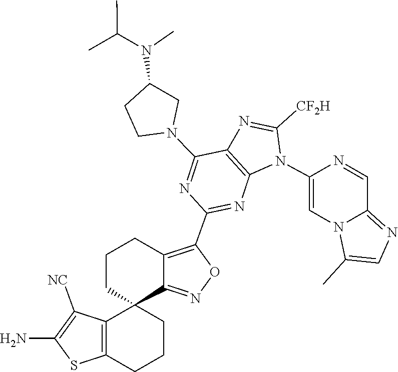 [SMILES: Cc1cnc2cnc(-n3c(C(F)F)nc4c(N5CC[C@H](N(C)C(C)C)C5)nc(-c5onc6c5CCC[C@@]65CCCc6sc(N)c(C#N)c65)nc43)cn12] | 1.57 | 724 | B |
| II-163 | 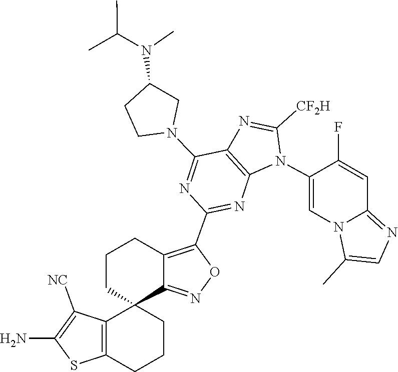 [SMILES: Cc1cnc2cc(F)c(-n3c(C(F)F)nc4c(N5CC[C@H](N(C)C(C)C)C5)nc(-c5onc6c5CCC[C@@]65CCCc6sc(N)c(C#N)c65)nc43)cn12] | 1.57 | 742 | B |
| II-164 | 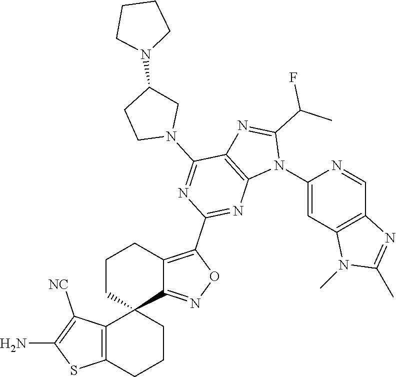 [SMILES: Cc1nc2cnc(-n3c(C(C)F)nc4c(N5CC[C@H](N6CCCC6)C5)nc(-c5onc6c5CCC[C@@]65CCCc6sc(N)c(C#N)c65)nc43)cc2n1C] | 1.50 | 733 | B |
| II-165 | 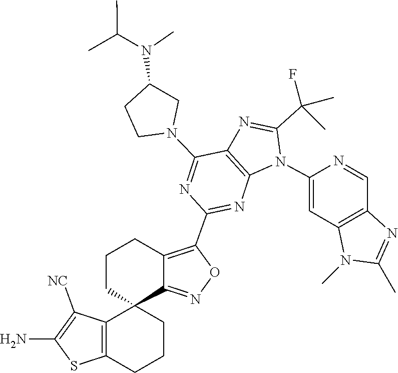 [SMILES: Cc1nc2cnc(-n3c(C(C)(C)F)nc4c(N5CC[C@H](N(C)C(C)C)C5)nc(-c5onc6c5CCC[C@@]65CCCc6sc(N)c(C#N)c65)nc43)cc2n1C] | 1.56 | 749 | B |
| II-166 | 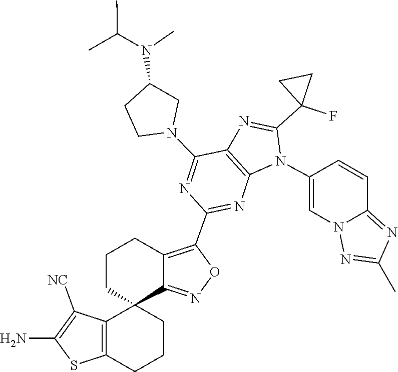 [SMILES: Cc1nc2ccc(-n3c(C4(F)CC4)nc4c(N5CC[C@H](N(C)C(C)C)C5)nc(-c5onc6c5CCC[C@@]65CCCc6sc(N)c(C#N)c65)nc43)cn2n1] | 1.59 | 733 | B |
| II-167 | 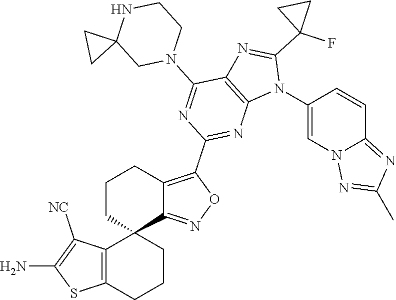 [SMILES: Cc1nc2ccc(-n3c(C4(F)CC4)nc4c(N5CCNC6(CC6)C5)nc(-c5onc6c5CCC[C@@]65CCCc6sc(N)c(C#N)c65)nc43)cn2n1] | 1.35 | 703 | B |
| II-168 | 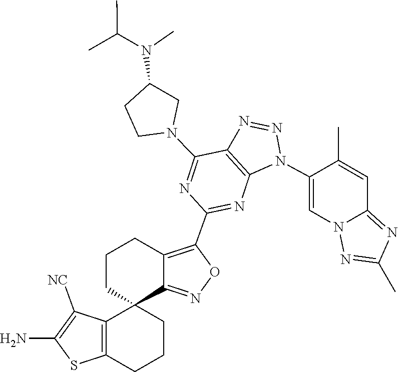 [SMILES: Cc1nc2cc(C)c(-n3nnc4c(N5CC[C@H](N(C)C(C)C)C5)nc(-c5onc6c5CCC[C@@]65CCCc6sc(N)c(C#N)c65)nc43)cn2n1] | 1.51 | 690 | B |
| II-169 | 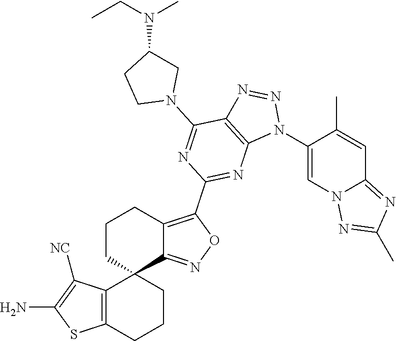 [SMILES: CCN(C)[C@H]1CCN(c2nc(-c3onc4c3CCC[C@@]43CCCc4sc(N)c(C#N)c43)nc3c2nnn3-c2cn3nc(C)nc3cc2C)C1] | 1.43 | 676 | B |
| II-170 | 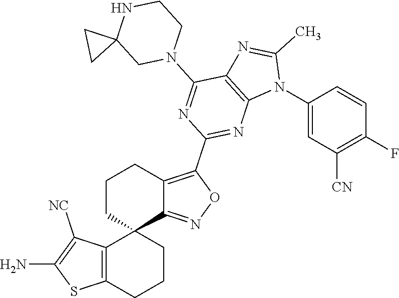 [SMILES: Cc1nc2c(N3CCNC4(CC4)C3)nc(-c3onc4c3CCC[C@@]43CCCc4sc(N)c(C#N)c43)nc2n1-c1ccc(F)c(C#N)c1] | 1.41 | 647 | B |
| II-171 | 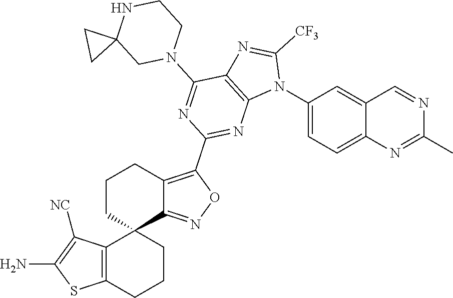 [SMILES: Cc1ncc2cc(-n3c(C(F)(F)F)nc4c(N5CCNC6(CC6)C5)nc(-c5onc6c5CCC[C@@]65CCCc6sc(N)c(C#N)c65)nc43)ccc2n1] | 1.30 | 670 | B |
| II-172 | 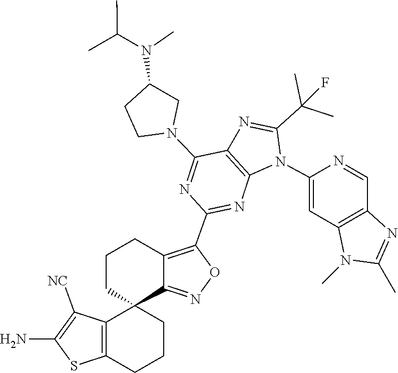 [SMILES: Cc1nc2cnc(-n3c(C(C)(C)F)nc4c(N5CC[C@H](N(C)C(C)C)C5)nc(-c5onc6c5CCC[C@@]65CCCc6sc(N)c(C#N)c65)nc43)cc2n1C] | 1.56 | 749 | B |
| II-173 | 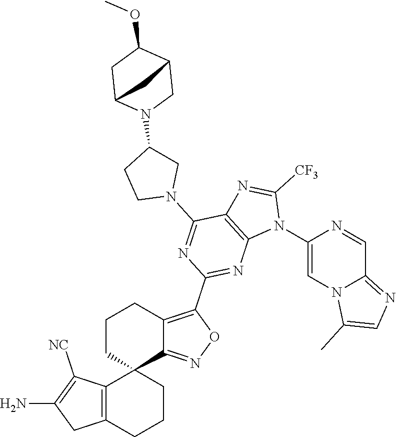 [SMILES: CO[C@@H]1C[C@@H]2C[C@H]1CN2[C@H]1CCN(c2nc(-c3onc4c3CCC[C@@]43CCCC4=C3C(C#N)=C(N)C4)nc3c2nc(C(F)(F)F)n3-c2cn3c(C)cnc3cn2)C1] | 1.53 | 797 | B |
| II-174 | 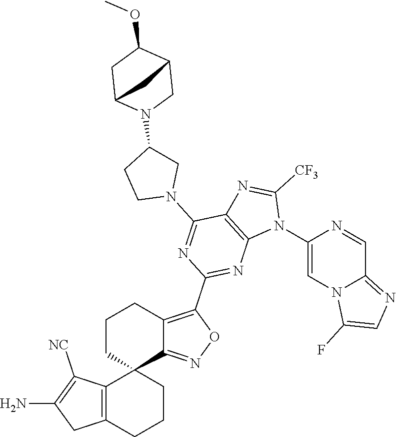 [SMILES: CO[C@@H]1C[C@@H]2C[C@H]1CN2[C@H]1CCN(c2nc(-c3onc4c3CCC[C@@]43CCCC4=C3C(C#N)=C(N)C4)nc3c2nc(C(F)(F)F)n3-c2cn3c(F)cnc3cn2)C1] | 1.56 | 801 | B |
| II-175 | 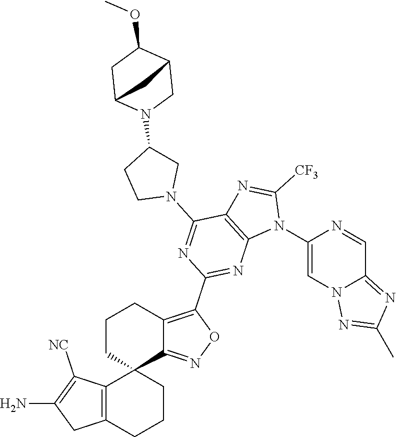 [SMILES: CO[C@@H]1C[C@@H]2C[C@H]1CN2[C@H]1CCN(c2nc(-c3onc4c3CCC[C@@]43CCCC4=C3C(C#N)=C(N)C4)nc3c2nc(C(F)(F)F)n3-c2cn3nc(C)nc3cn2)C1] | 1.58 | 798 | B |
| II-176 | 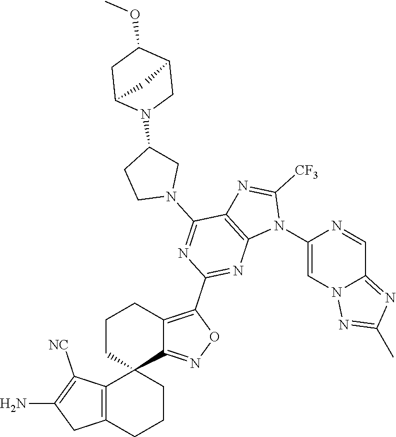 [SMILES: CO[C@H]1C[C@H]2C[C@@H]1CN2[C@H]1CCN(c2nc(-c3onc4c3CCC[C@@]43CCCC4=C3C(C#N)=C(N)C4)nc3c2nc(C(F)(F)F)n3-c2cn3nc(C)nc3cn2)C1] | 1.58 | 798 | B |
| II-177 | 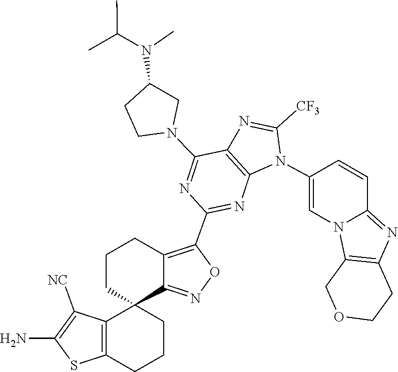 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nc4c(n3c2)COCC4)C1] | 1.60 | 784 | B |
| II-178 | 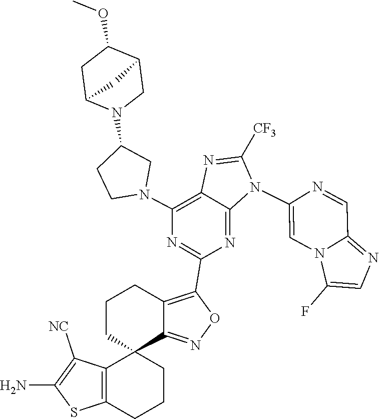 [SMILES: CO[C@H]1C[C@H]2C[C@@H]1CN2[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] | 1.55 | 801 | B |
| II-179 | 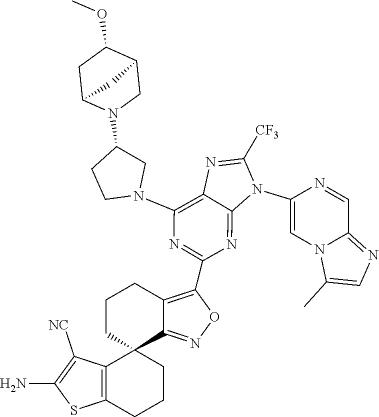 [SMILES: CO[C@H]1C[C@H]2C[C@@H]1CN2[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] | 1.53 | 797 | B |
| II-180 | 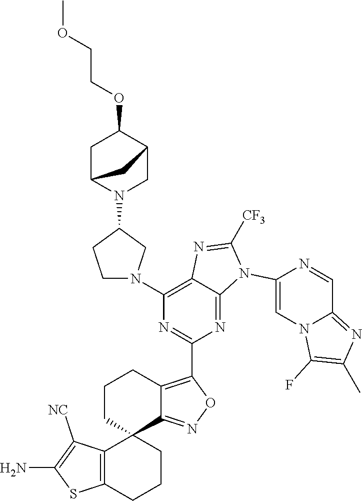 [SMILES: COCCO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(F)c(C)nc3cn2)C1] | 1.16 | 859 | A |
| II-181 | 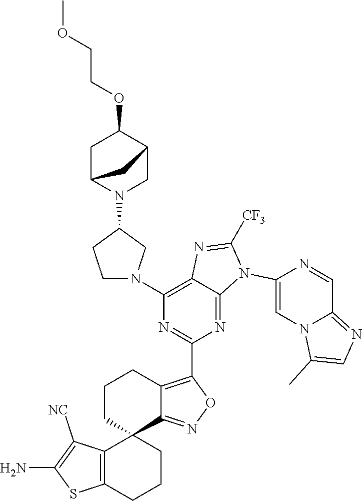 [SMILES: COCCO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] | 1.50 | 841 | B |

### Reductive Amination

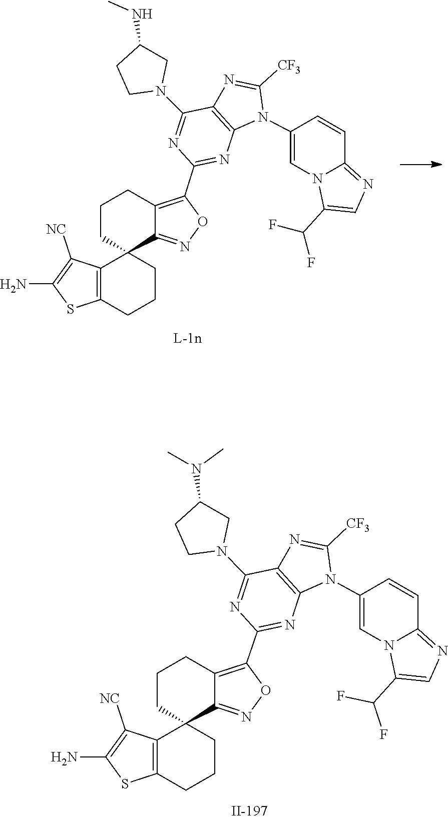 [SMILES: CN[C@H]1CCN(c2nc(-c3onc4c3CCC[C@@]43CCCc4sc(N)c(C#N)c43)nc3c2nc(C(F)(F)F)n3-c2ccc3ncc(C(F)F)n3c2)C1 ; CN(C)[C@H]1CCN(c2nc(-c3onc4c3CCC[C@@]43CCCc4sc(N)c(C#N)c43)nc3c2nc(C(F)(F)F)n3-c2ccc3ncc(C(F)F)n3c2)C1]

A solution of L-1n (90 mg, 0122 mmol, 1.0 equiv.) in methanol is stirred with formaldehyde (11.9 mg, 0.147 mmol, 1.2 equiv.) and a drop of acetic acid for 5 minutes. Sodium cyanoborohydride solution (0.24 mL, 0.24 mmol, 1.0 equiv.) is added and the reaction is allowed to proceed for 30 minutes. The reaction mixture is basified with ammonia, filtered and purified by chromatography to give the desired product 11-197 (H PLC method A, tᵣₑₜ=1.471 min; [M+H]=751).

The following final compounds II (Table 26) are obtained analogously using the indicated carbonyl component. If no carbonyl component is shown, acetone is used. The crude product is purified by chromatography if necessary.

TABLE 26

| | | tᵣₑₜ | | HPLC | Carbonyl |
|---|---|---|---|---|---|
| # | structure | [min] | [M + H]⁺ | method | component |
| II-182 | 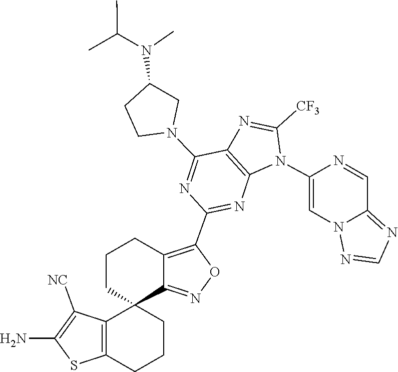 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3ncnc3cn2)C1] | 1.61 | 730 | B | 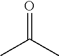 |
| II-183 | 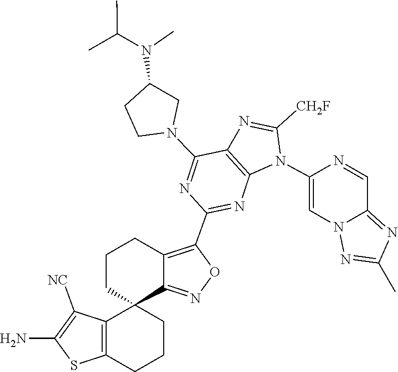 | 1.54 | 708 | B | 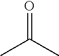 |
| II-184 | 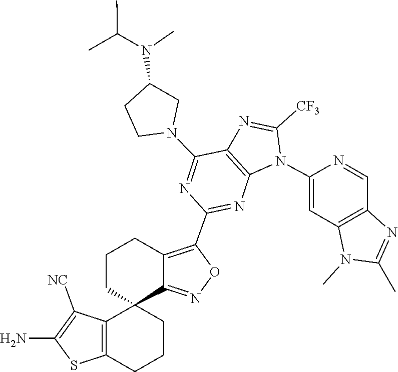 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] | 1.58 | 757 | B | 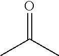 |
| II-185 | 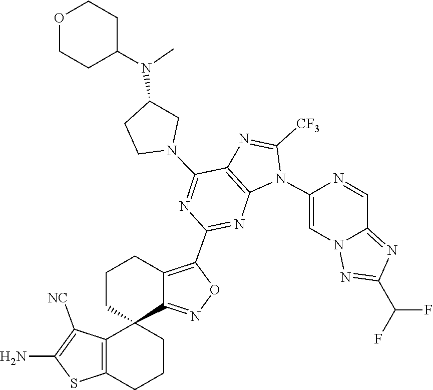 [SMILES: CN(C1CCOCC1)[C@H]1CCN(c2nc(-c3onc4c3CCC[C@@]43CCCc4sc(N)c(C#N)c43)nc3c2nc(C(F)(F)F)n3-c2cn3nc(C(F)F)nc3cn2)C1] | 1.56 | 822 | B | 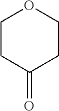 |
| II-186 | 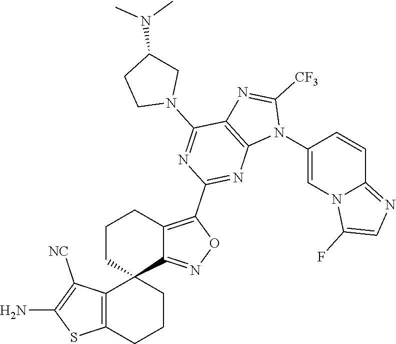 [SMILES: CN(C)[C@H]1CCN(c2nc(-c3onc4c3CCC[C@@]43CCCc4sc(N)c(C#N)c43)nc3c2nc(C(F)(F)F)n3-c2ccc3ncc(F)n3c2)C1] | 1.50 | 718 | B | 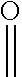 |
| II-187 | 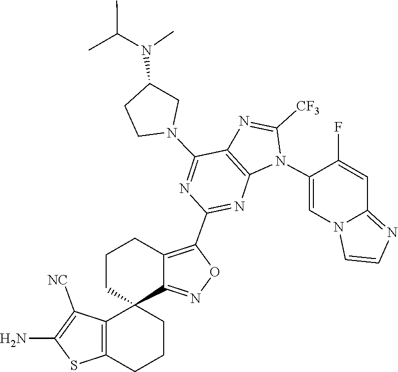 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3ccnc3cc2F)C1] | 1.63 | 746 | B | 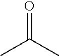 |
| II-188 | 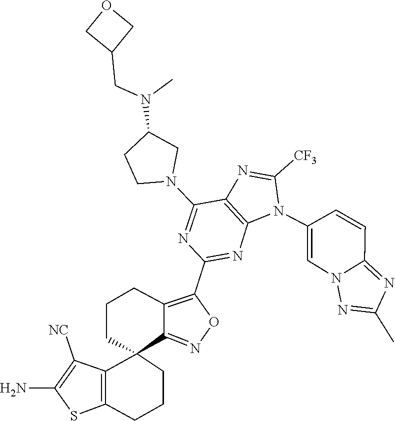 [SMILES: Cc1nc2ccc(-n3c(C(F)(F)F)nc4c(N5CC[C@H](N(C)CC6COC6)C5)nc(-c5onc6c5CCC[C@@]65CCCc6sc(N)c(C#N)c65)nc43)cn2n1] | 1.42 | 771 | B | 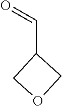 |
| II-189 | 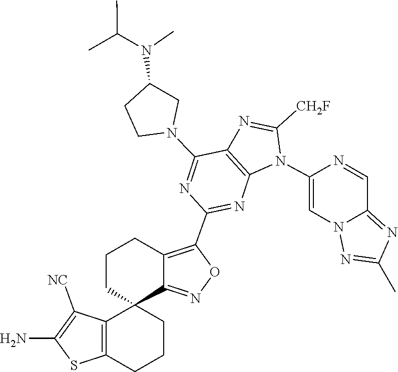 | 1.60 | 726 | B | 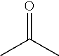 |
| II-190 | 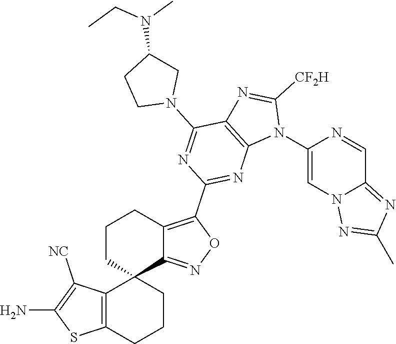 [SMILES: CCN(C)[C@H]1CCN(c2nc(-c3onc4c3CCC[C@@]43CCCc4sc(N)c(C#N)c43)nc3c2nc(C(F)F)n3-c2cn3nc(C)nc3cn2)C1] | 1.52 | 712 | B | 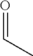 |
| II-191 | 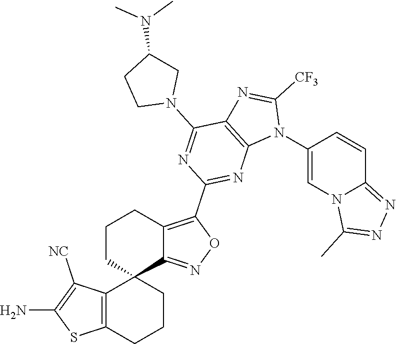 [SMILES: Cc1nnc2ccc(-n3c(C(F)(F)F)nc4c(N5CC[C@H](N(C)C)C5)nc(-c5onc6c5CCC[C@@]65CCCc6sc(N)c(C#N)c65)nc43)cn12] | 1.36 | 715 | B | 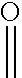 |
| II-192 | 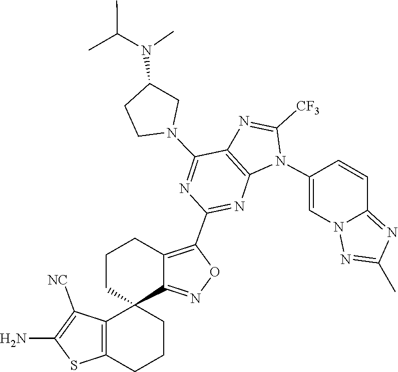 [SMILES: Cc1nc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1] | 1.61 | 743 | B | 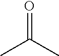 |
| II-193 | 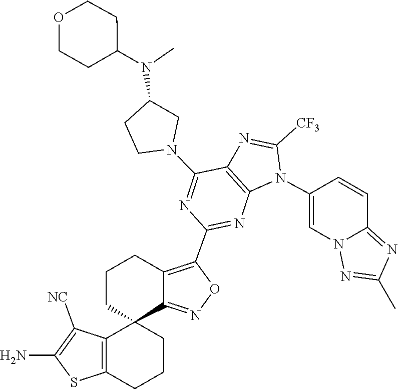 [SMILES: Cc1nc2ccc(-n3c(C(F)(F)F)nc4c(N5CC[C@H](N(C)C6CCOCC6)C5)nc(-c5onc6c5CCC[C@@]65CCCc6sc(N)c(C#N)c65)nc43)cn2n1] | 1.47 | 785 | B | 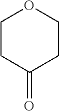 |
| II-194 | 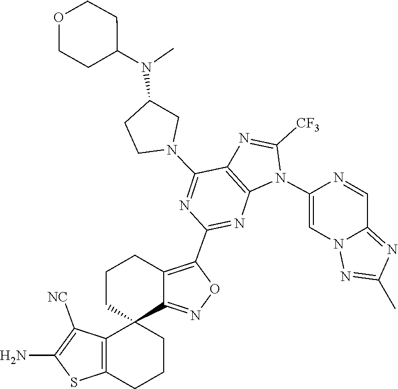 [SMILES: Cc1nc2cnc(-n3c(C(F)(F)F)nc4c(N5CC[C@H](N(C)C6CCOCC6)C5)nc(-c5onc6c5CCC[C@@]65CCCc6sc(N)c(C#N)c65)nc43)cn2n1] | 1.51 | 786 | B | 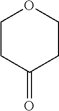 |
| II-195 | 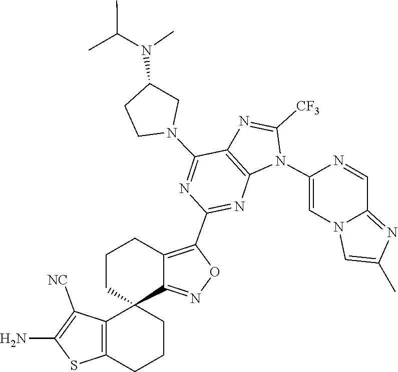 [SMILES: Cc1cn2cc(-n3c(C(F)(F)F)nc4c(N5CC[C@H](N(C)C(C)C)C5)nc(-c5onc6c5CCC[C@@]65CCCc6sc(N)c(C#N)c65)nc43)ncc2n1] | 1.60 | 743 | B | 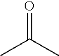 |
| II-196 | 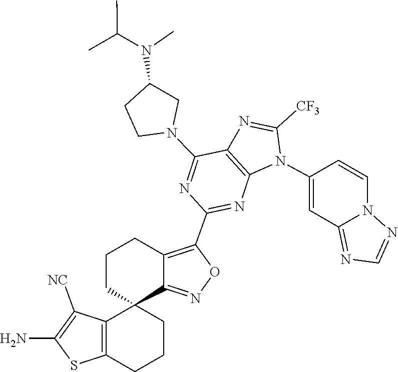 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccn3ncnc3c2)C1] | 1.59 | 729 | B | 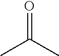 |

### Assay Description and Data

### KRAS TR-FRET Assays

This assay measures the inhibitory effect of compounds on KRAS::CRAF protein::protein interactions in the presence of GTP using Time-resolved fluorescence energy transfer (TR-FRET).

The following wildtype or mutant alleles of KRAS and CRAF protein are used at the given concentrations:

KRAS (WT) 1-169, N-terminal 6His-tag, C-terminal avi-tag (Boehringer Ingelheim in house protein prep); final assay concentration 15 nM.

KRAS (G12D) 1-169, N-terminal 6His-tag, C-terminal avi-tag (Xtal BioStructures, Inc.); final assay concentration 15 nM,

CRAF Ras binding domain, GST-tag, TEV cleavage site (Boehringer Ingelheim in house protein prep); final assay concentration 15 nM.

Test compounds dissolved in DMSO are dispensed onto assay plates (Proxiplate 384 PLUS, white, PerkinElmer; 6008289) using an Access Labcyte Workstation with the Labcyte Echo 55x. For the chosen highest assay concentration of 100 μM, 150 nl of compound solution is transferred from a 10 mM DMSO compound stock solution. A series of eleven fivefold dilutions per compound is transferred to the assay plate, compound dilutions are tested in duplicates. DMSO is added as backfill to a total volume of 150 nl.

The assay runs on a fully automated robotic system. To 150nl of compound dilution 15 μl of a mix including the respective KRAS allele, CRAF (e.g. KRAS (G12D)::CRAF; final assay concentrations see above), GTP nucleotide (Sigma G8877; final assay concentration 10 μM), Lance Eu-W1024 labeled Streptavidin (PerkinElmer, Cat No AD0063; final assay concentration 1.5 nM) and Anti-GST surelight APC (PerkinElmer, Cat No AD0059G; final assay concentration 30 nM) in assay buffer (1×PBS, 0.1% BSA, 0.05% Tween 20) are added into columns 1-23 and 15 μL of the solution without the respective KRAS allele are added to row 24.

Plates are kept at room temperature in a darkened incubator. After 60 minutes incubation time the signal is measured in a PerkinElmer Envision HTS Multilabel Reader using the TR-FRET LANCE Ultra specs from PerkinElmer.

Each plate contains 16 wells of a negative control (diluted DMSO instead of test compound; w respective KRAS allele; column 23) and 16 wells of a positive control (diluted DMSO instead of test compound; w/o respective KRAS allele; column 24).

IC50 values are calculated and analyzed with Boehringer Ingelheim's MEGALAB IC50 application using a 4 parametric logistic model.

### Ba/F3 Cell Model Generation and Proliferation Assay

Ba/F3 cells are ordered from DSMZ (ACC300, Lot17) and grown in RPMI-1640 (ATCC 30-2001)+10% FCS+10 ng/mL IL-3 at 37° C. in 5% CO₂ atmosphere. A plasmid containing the KRASG12D sequence is obtained from GeneScript. To generate a KRASG12D-dependent Ba/F3 model, Ba/F3 cells are transduced with a retrovirus containing a vector that harbors the KRASG12D isoform. Platinum-E cells (Cell Biolabs) are used for retrovirus packaging. Retrovirus is added to Ba/F3 cells. To ensure infection, 4 μg/mL polybrene is added and cells are spinfected. Infection efficiency is confirmed by measuring GFP-positive cells using a cell analyzer. Cells with an infection efficiency of 10% to 20% are further cultivated and puromycin selection with 1 μg/mL is initiated. As a control, parental Ba/F3 cells are used to show selection status. Selection is considered successful when parental Ba/F3 cells cultures died. To evaluate the transforming potential of the KRASG12D mutation, the growth medium is no longer supplemented with IL-3. Ba/F3 cells harboring the empty vector are used as a control. Approximately ten days before conducting the experiments, puromycin is left out.

For proliferation assays, Ba/F3 cells are seeded into 384-well plates at 1.5×10³ cells/60 μL in growth media (RPMI-1640+10% FCS). Compounds (10 mM stock in DMSO) are added at logarithmic dose series using the ECHO acoustic liquid handler system (Beckman Coulter), normalizing for added DMSO and including DMSO controls. All compound treatments are performed in technical duplicates. For the TO time point measurement, untreated cells are analyzed at the time of compound addition. Treated cells are incubated for 72 h at 37° C. with 5% CO₂ and cell viability is measured in the PerkinElmer Envision Multimode Reader using AlamarBlue™ (ThermoFisher) viability stain. Viability (stated as percent of control) is defined as relative fluorescence units RFU of each well divided by the RFU of cells in DMSO controls. IC₅₀ values are determined from viability measurements by non-linear regression using a four-parameter model.

IC₅₀ values of representative compounds according to the invention measured with this assay are presented in Table 27.

Additional Proliferation Assays with Mutant Cancer Cell Lines

### AsPC-1 CTG Proliferation Assay (120 h) (Pancreatic Cancer, G12D)

AsPC-1 cells (ATCC CRL-1682) are dispensed into black 384-well plates, flat and clear bottom (Greiner, PNr. 781091) at a density of 500 cells per well in 60 μl RPMI ATCC-Formulation (PAN P04-18047)+10% FCS (fetal calf serum, HyClone, SH30084.03). Cells are incubated overnight at 37° C. in a humidified tissue culture incubator at 5% CO₂. Compounds (10 mM stock in DMSO) are added at logarithmic dose series using the ECHO acoustic liquid handler system (Beckman Coulter), normalizing for added DMSO and including DMSO controls. All compound treatments are performed in technical duplicates. For the TO time point measurement, untreated cells are analyzed at the time of compound addition. Plates are incubated for 120 h, and cell viability is measured using CellTiter-Glo® 2.0 Cell Viability Reagent (Promega G9243). Viability (stated as percent of control) is defined as relative luminescence units RLU of each well divided by the RLU of cells in DMSO controls. IC₅₀ values are determined from viability measurements by non-linear regression using a four-parameter model.

### GP2d CTG Proliferation Assay (120 h) (Adenocarcinoma, KRAS G12D Mutant)

GP2d cells (ECACC No. 95090714) are dispensed into black 384-well plates, flat and clear bottom (Greiner, PNr. 781091) at a density of 500 cells per well in 60 μl DMEM (Sigma D6429)+10% FCS (fetal calf serum, HyClone, SH30084.03). Cells are incubated overnight at 37° C. in a humidified tissue culture incubator at 5% CO₂. Compounds (10 mM stock in DMSO) are added at logarithmic dose series using the ECHO acoustic liquid handler system (Beckman Coulter), normalizing for added DMSO and including DMSO controls. All compound treatments are performed in technical duplicates. For the TO time point measurement, untreated cells are analyzed at the time of compound addition. Plates are incubated for 120 h, and cell viability is measured using CellTiter-Glo® 2.0 Cell Viability Reagent (Promega G9243). Viability (stated as percent of control) is defined as relative luminescence units RLU of each well divided by the RLU of cells in DMSO controls. IC₅₀ values are determined from viability measurements by non-linear regression using a four-parameter model.

Table of example compounds disclosed herein contain IC₅₀ values determined using the above assays (see Table 27)

TABLE 27

| | | KRASG12D::CRAF | KRAS_WT::CRAF | ASPC-1 | GP2D |
|---|---|---|---|---|---|
| | BAF3_KRAS_G12D | GTP | GTP | IC₅₀ | IC50 |
| # | IC₅₀ (nM) | IC₅₀ (nM) | IC₅₀ (nM) | (nM) | (nM) |
| II-1 | 2.6 | 20.7 | 78 | 26 | 2 |
| II-2 | 3.3 | 16.0 | 66 | 20 | 3 |
| II-3 | 6.1 | 27.7 | 117 | 237 | 17 |
| II-4 | 8.4 | 35.5 | 180 | 701 | 4 |
| II-5 | 13.3 | 38.4 | 214 | 92 | 11 |
| II-6 | 5.3 | 110.0 | 675 | 162 | 13 |
| II-7 | 7.3 | 65.6 | 527 | 69 | 7 |
| II-8 | 131.1 | 835.2 | 8536 | 815 | 187 |
| II-9 | 273.4 | 1467.5 | >20000 | 770 | 304 |
| II-10 | 207.6 | 5648.7 | >100000 | 1801 | >1000 |
| II-11 | 17.5 | 47.3 | 2879 | 246 | 70 |
| II-12 | 8.6 | 30.2 | 2318 | 158 | 13 |
| II-13 | 26.0 | 110.1 | 1729 | 170 | 37 |
| II-14 | 9.3 | 14.4 | 336 | 223 | 5 |
| II-15 | 14.3 | 62.9 | 1611 | 102 | 18 |
| II-16 | 10.5 | 55.6 | 1452 | 148 | 31 |
| II-17 | 3.0 | 12.6 | 469 | 43 | 14 |
| II-18 | 12.5 | 25.0 | 947 | 44 | 12 |
| II-19 | 2.4 | 11.5 | 699 | 31 | 3 |
| II-20 | 1.8 | 12.6 | 862 | 30 | 3 |
| II-21 | 10.9 | 23.0 | 1612 | 41 | 10 |
| II-22 | 4.7 | 18.1 | 62 | 13 | 3 |
| II-23 | 3.0 | 10.9 | 33 | 56 | 3 |
| II-24 | 4.0 | 36.1 | 115 | 284 | 9 |
| II-25 | 2.8 | 20.3 | 79 | 35 | 3 |
| II-26 | 2.8 | 18.5 | 75 | 23 | 3 |
| II-27 | 10.1 | 61.3 | 253 | 109 | 8 |
| II-28 | 4.6 | 39.9 | 166 | 416 | 5 |
| II-29 | 7.6 | 39.8 | 199 | 50 | 12 |
| II-30 | 17.2 | 100.4 | 504 | 159 | 5 |
| II-31 | 3.4 | 45.5 | 243 | 149 | 6 |
| II-32 | 8.2 | 72.2 | 456 | 75 | 15 |
| II-33 | 164.1 | 2316.0 | 24501 | 1542 | 245 |
| II-34 | 187.5 | 1010.6 | 37971 | 935 | 168 |
| II-35 | 139.7 | 1062.8 | >100000 | 2804 | 149 |
| II-36 | 9.4 | 52.4 | 554 | 64 | 8 |
| II-37 | 6.6 | 20.0 | 1188 | 163 | |
| II-38 | 4.5 | 17.7 | 2131 | 30 | 4 |
| II-39 | 11.9 | 82.2 | 724 | 472 | 28 |
| II-40 | 12.1 | 63.6 | 4954 | 87 | 18 |
| II-41 | 6.2 | 10.8 | 245 | 82 | 8 |
| II-42 | 9.1 | 36.1 | 5472 | 188 | 8 |
| II-43 | 6.7 | 17.0 | 553 | 142 | 8 |
| II-44 | 11.1 | 20.5 | 763 | 135 | 38 |
| II-45 | 7.0 | 21.2 | 798 | 55 | 23 |
| II-46 | 21.3 | 26.1 | 1038 | 62 | 24 |
| II-47 | 14.1 | 48.4 | 2131 | 285 | 45 |
| II-48 | 3.9 | 16.6 | 932 | 76 | 8 |
| II-49 | 7.9 | 35.7 | 3881 | 135 | 12 |
| II-50 | 5.9 | 26.8 | 1606 | 51 | 11 |
| II-51 | 8.7 | 42.9 | 3626 | 358 | 12 |
| II-52 | 2.9 | 8.0 | 1028 | 5 | 2 |
| II-53 | 2.1 | 10.6 | 1170 | 9 | 3 |
| II-54 | 1.9 | 14.8 | 1145 | 10 | 5 |
| II-55 | 2.6 | 7.0 | 938 | 12 | 2 |
| II-56 | 6.2 | 8.0 | 897 | 14 | 2 |
| II-57 | 3.6 | 14.2 | 1436 | 15 | 3 |
| II-58 | 3.1 | 12.7 | 2097 | 17 | 4 |
| II-59 | 1.5 | 31.0 | 3777 | 17 | 5 |
| II-60 | 2.4 | 4.9 | 1195 | 17 | 2 |
| II-61 | 18.0 | 122.9 | >100000 | 18 | 67 |
| II-62 | 1.4 | 18.3 | 4843 | 19 | 3 |
| II-63 | 3.3 | 27.5 | 2184 | 20 | 8 |
| II-64 | 8.6 | 37.8 | 9459 | 20 | 3 |
| II-65 | 1.4 | 9.6 | 1324 | 20 | 3 |
| II-66 | 1.3 | 17.9 | 5020 | 20 | 3 |
| II-67 | 3.6 | 9.0 | 799 | 20 | 2 |
| II-68 | 5.8 | 26.1 | 5106 | 21 | 5 |
| II-69 | 2.5 | 10.8 | 870 | 21 | 4 |
| II-70 | 6.8 | 121.3 | >100000 | 23 | 36 |
| II-71 | 2.9 | 37.3 | >20000 | 23 | 10 |
| II-72 | 5.7 | 9.6 | 984 | 24 | 9 |
| II-73 | 2.1 | 8.0 | 2482 | 25 | 4 |
| II-74 | 2.7 | 27.1 | 11848 | 25 | 7 |
| II-75 | 1.9 | 11.8 | 2336 | 25 | 3 |
| II-76 | 5.3 | 20.8 | 714 | 27 | 7 |
| II-77 | 2.6 | 28.4 | >100000 | 28 | 3 |
| II-78 | 4.9 | 63.6 | >20000 | 28 | 9 |
| II-79 | 4.2 | 12.6 | 1080 | 29 | 6 |
| II-80 | 4.9 | 13.3 | 1122 | 29 | 7 |
| II-81 | 3.1 | 29.5 | 2954 | 29 | 9 |
| II-82 | 1.6 | 19.2 | 1226 | 30 | 2 |
| II-83 | 5.0 | 18.9 | 1968 | 30 | 4 |
| II-84 | 2.4 | 11.4 | 2003 | 31 | 1 |
| II-85 | 4.8 | 8.9 | 1035 | 31 | 200 |
| II-86 | 3.3 | 14.5 | 1023 | 32 | 8 |
| II-87 | 1.6 | 13.2 | 817 | 33 | 3 |
| II-88 | 3.6 | 12.0 | 1291 | 34 | 7 |
| II-89 | 1.6 | 21.0 | 9588 | 35 | 4 |
| II-90 | 3.0 | 18.3 | 4512 | 35 | 4 |
| II-91 | 5.2 | 46.9 | 3072 | 35 | 11 |
| II-92 | 7.2 | 26.0 | 2651 | 36 | 6 |
| II-93 | 3.6 | 27.3 | 4879 | 37 | 7 |
| II-94 | 6.2 | 18.6 | 2811 | 37 | 5 |
| II-95 | 2.0 | 41.6 | >20000 | 38 | 11 |
| II-96 | 3.5 | 21.7 | 2345 | 38 | 11 |
| II-97 | 5.1 | 11.3 | 2172 | 39 | 3 |
| II-98 | 5.5 | 20.5 | 1510 | 39 | 4 |
| II-99 | 4.8 | 12.4 | 1802 | 40 | 3 |
| II-100 | 10.2 | 8.7 | 731 | 41 | 3 |
| II-101 | 9.6 | 21.7 | 2227 | 41 | 10 |
| II-102 | 3.4 | 51.9 | >20000 | 41 | 9 |
| II-103 | 4.0 | 11.1 | 1647 | 42 | 2 |
| II-104 | 3.4 | 27.3 | 9264 | 42 | 9 |
| II-105 | 6.9 | | | 43 | 21 |
| II-106 | 3.7 | 19.2 | 3132 | 44 | 8 |
| II-107 | 5.0 | 61.4 | 4067 | 44 | 15 |
| II-108 | 2.1 | 20.7 | 3094 | 45 | 4 |
| II-109 | 12.9 | 20.6 | 3131 | 50 | 19 |
| II-110 | 5.8 | 43.8 | 4052 | 51 | 8 |
| II-111 | 3.8 | 46.8 | >100000 | 53 | 15 |
| II-112 | 9.3 | 59.6 | 3363 | 53 | 49 |
| II-113 | 6.8 | 37.9 | 3280 | 53 | 25 |
| II-114 | 2.0 | 6.2 | 689 | 53 | 4 |
| II-115 | 5.1 | 46.3 | 6011 | 55 | 6 |
| II-116 | 7.0 | 27.8 | 4887 | 55 | 7 |
| II-117 | 3.9 | 16.8 | 1681 | 55 | 4 |
| II-118 | 4.0 | 14.0 | 2257 | 54 | 9 |
| II-119 | 2.6 | 6.4 | 2563 | 58 | 5 |
| II-120 | 15.2 | 32.6 | 4346 | 58 | 15 |
| II-121 | 8.9 | 56.4 | 3607 | 60 | 7 |
| II-122 | 4.5 | 13.3 | 871 | 61 | 43 |
| II-123 | 6.7 | 27.6 | 6065 | 62 | 19 |
| II-124 | 7.4 | 67.3 | 9702 | 63 | 24 |
| II-125 | 5.1 | 38.9 | 10660 | 63 | 7 |
| II-126 | 6.6 | 46.6 | 6107 | 67 | 18 |
| II-127 | 10.3 | 83.0 | >100000 | 69 | 27 |
| II-128 | 11.7 | 44.4 | 1716 | 69 | 14 |
| II-129 | 4.7 | 27.6 | 4008 | 70 | 11 |
| II-130 | 8.1 | 58.9 | >100000 | 72 | 12 |
| II-131 | 10.3 | 28.8 | 1531 | 73 | 14 |
| II-132 | 5.7 | 28.4 | 3039 | 73 | 17 |
| II-133 | 10.3 | 53.2 | 2535 | 78 | 10 |
| II-134 | 9.8 | 53.4 | 21900 | 78 | 9 |
| II-135 | 10.5 | 15.3 | 1811 | 79 | 4 |
| II-136 | 7.4 | 7.6 | 933 | 82 | 2 |
| II-137 | 4.3 | 29.6 | 2126 | 84 | 6 |
| II-138 | 11.9 | 41.3 | 9876 | 85 | 16 |
| II-139 | 8.5 | 43.1 | 6574 | 85 | 26 |
| II-140 | 10.5 | 63.2 | 15393 | 87 | 16 |
| II-141 | 5.1 | 70.0 | 6085 | 87 | 42 |
| II-142 | 18.3 | 12.5 | 934 | 94 | 5 |
| II-143 | 7.2 | 76.9 | 4546 | 95 | 53 |
| II-144 | 13.5 | 51.3 | 2718 | 95 | 60 |
| II-145 | 7.6 | 37.3 | 1737 | 99 | 15 |
| II-146 | 7.2 | 46.5 | 7437 | 107 | 27 |
| II-147 | 11.0 | 138.3 | >100000 | 113 | 39 |
| II-148 | 3.4 | 49.8 | 3171 | 119 | 10 |
| II-149 | 15.8 | 65.0 | 3637 | 120 | 68 |
| II-150 | 6.5 | 56.8 | 4583 | 129 | 23 |
| II-151 | 15.6 | 80.0 | 5494 | 135 | 28 |
| II-152 | 8.8 | 79.6 | >20000 | 154 | 33 |
| II-153 | 5.2 | 87.0 | >100000 | 155 | 14 |
| II-154 | 14.4 | 117.5 | >100000 | 156 | 20 |
| II-155 | 12.7 | 73.5 | 3705 | 170 | 49 |
| II-156 | 8.3 | 100.2 | >100000 | 172 | 33 |
| II-157 | 15.4 | 87.4 | >20000 | 174 | 37 |
| II-158 | 11.2 | 161.5 | >100000 | 213 | 72 |
| II-159 | 10.1 | 107.4 | 6573 | 219 | 67 |
| II-160 | 4.2 | 34.1 | >4000 | 21 | 17 |
| II-161 | 3.9 | 18.1 | 1860 | 22 | 34 |
| II-162 | 4.6 | 13.1 | 807 | 22 | 18 |
| II-163 | 7.9 | 35.2 | 26766 | 55 | 6 |
| II-164 | 10.2 | 19.2 | 1445 | 23 | 2 |
| II-165 | 3.9 | 10.9 | 883 | 41 | 1 |
| II-166 | 7.6 | 38.7 | 5413 | 75 | 23 |
| II-167 | 11.9 | 48.6 | 249 | 42 | 5 |
| II-168 | 12.9 | 52.1 | 4404 | 83 | 25 |
| II-169 | 35.2 | 94.8 | >4000 | 307 | 34 |
| II-170 | 23.0 | 256.2 | 3550 | 278 | 47 |
| II-171 | 16.8 | 50.1 | 450 | 108 | 8 |
| II-172 | 3.9 | 10.9 | 883 | 41 | 1 |
| II-173 | | 18.8 | 1052 | 8 | 1 |
| II-174 | | 10.3 | 1474 | 22 | 2 |
| II-175 | 2.0 | 16.3 | 3148 | 24 | 4 |
| II-176 | 1.9 | 15.1 | 2109 | 30 | 5 |
| II-177 | 0.9 | 19.9 | 16617 | 31 | 2 |
| II-178 | | 11 | 1364 | 33 | 5 |
| II-179 | | 9 | 886 | 11 | 2 |
| II-180 | | 9 | 1721 | 13 | 2 |
| II-181 | | 7 | 594 | 8 | 1 |
| II-182 | 9.0 | 19.1 | 3519 | 41 | 11 |
| II-183 | 2.4 | 17.3 | 2067 | 18 | 13 |
| II-184 | 2.7 | 13.6 | 647 | 9 | 2 |
| II-185 | 7.8 | 91.7 | >100000 | 90 | 24 |
| II-186 | 15.5 | 32.4 | 2162 | 125 | 21 |
| II-187 | 13.2 | 97.5 | >20000 | 189 | 35 |
| II-188 | 14.3 | 53.0 | 4048 | 244 | 27 |
| II-189 | 4.4 | 23.9 | 4781 | 41 | 11 |
| II-190 | 13.0 | 41.7 | 4522 | 101 | 32 |
| II-191 | 9.8 | 8.7 | 311 | 64 | 7 |
| II-192 | 1.1 | 9.7 | 1582 | 17 | 3 |
| II-193 | 1.3 | 19.3 | 4853 | 18 | 4 |
| II-194 | 5.2 | 40.2 | 8949 | 25 | 12 |
| II-195 | 7.5 | 18.5 | 1766 | 36 | 10 |
| II-196 | 3.6 | 22.7 | 11293 | 38 | 10 |
| II-197 | 6.5 | 20.0 | 658 | 93 | 8 |

### RAT PK Data

PK studies are performed in male Han Wistar rats to measure plasma clearance and volume of distribution (Vss) in vivo.

Sample preparation for plasma samples (protein precipitation performed using 96-well plate) was performed in general as following:

An aliquot of 10 μL plasma sample (and calibration standard, quality control, dilution quality control (if available), single blank, and double blank sample) were added to the 96-well plate respectively. Each aliquout (except the double blank) was quenched with 300 μL of IS1 (double blank samples were quenched with 300 μL of ACN), followed by vortex-mixing for 10 min at 800 rpm and centrifugation for 15 min at 3220×g at 4° C. After centrifugation, an aliquot of 50 μL supernatant was transferred to another 96-well plate and again centrifuged for 5 min at 3220×g at 4° C.

LC-MS/MS method

| MS | Sciex Triple Quad 6500+ | |
|---|---|---|
| MS conditions | ESI: positive; MRM detection | |
| Column | ACQUITY UPLC HSS T3 1.8 μm | |
| | 2.1 × 50 mm column | |
| Column temperature | 45° C. | |
| Solvent | A: 0.1% formic acid in water | |
| | B: 0.1% formic acid in ACN | |
| Flow | 0.60 mL/min | |
| Gradient | 0.00-1.30 min | 10% to 95% B |
| | 1.50-1.51 min | 95% B |
| | 1.51-1.60 min | 95% to 10% B |

PK parameters (CL, Vss) were calculated with entimiCE ToxKin application using trapezoidal rule (linear extrapolation for c0; logarithmic extrapolation for calculation of trapezoidterminal phase). For extrapolation to infinity, regression line rule using last 3 concentration values was applied.

TABLE 28

| | Dose | Plasma Clearance | Vss |
|---|---|---|---|
| # | [mg/kg] | [ml/min/kg] | [l/kg] |
| II-24 | 5 | 6.0 | 1.8 |
| II-45 | 5 | 13.2 | 7.1 |
| II-50 | 5 | 12.6 | 5.3 |
| II-60 | 5 | 30.0 | 10.7 |
| II-69 | 5 | 27.0 | 5.8 |
| II-75 | 5 | 25.0 | 10.9 |
| II-79 | 5 | 21.0 | 5.7 |
| II-80 | 5 | 14.0 | 5.0 |
| II-87 | 5 | 20.8 | 5.8 |
| II-118 | 5 | 16.4 | 5.9 |
| II-122 | 5 | 22.0 | 6.4 |
| II-148 | 5 | 3.4 | 1.6 |
| II-170 | 5 | 28.0 | 6.6 |
| II-171 | 5 | 24.9 | 4.1 |
| II-173 | 1 | 2.3 | 0.9 |
| II-174 | 0.5 | 6.2 | 3.9 |
| II-175 | 1 | 13.9 | 6.5 |
| II-176 | 1 | 22.2 | 7.1 |
| II-184 | 5 | 27.0 | 5.5 |
| II-192 | 5 | 24.0 | 7.7 |

1. A compound of the formula (I) 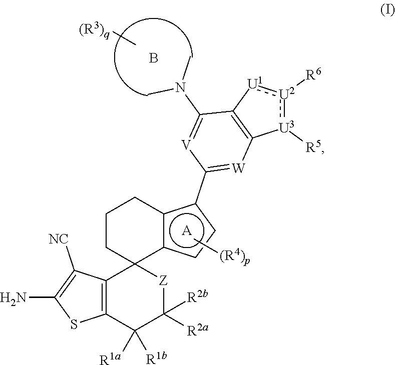
wherein
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
and/or, optionally, one of R^{1a} or R^{1b} and one of R^{2a} or R^{2b} together with the carbon atoms they are attached to form a cyclopropane ring;
Z is —(CR^{7a}R^{7b})ₙ—;
each R^{7a} and R^{7b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocyclyl;
or R^{7a} and R^{7b} together with the carbon atom they are attached to form a cyclopropane ring;
n is selected from the group consisting of 0, 1 and 2;
W is nitrogen (—N═) or —CH═;
V is nitrogen (—N═) or —CH═;
ring A is a ring selected from the group consisting of pyrrole, furan, thiophene, imidazole, pyrazole, oxazole, isoxazole, thiazole, isothiazole and triazole;
each R⁴, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocyclyl;
p is selected from the group consisting of 0, 1, 2 and 3;
U¹ is nitrogen (—N═) or sulphur (—S—);
U² is nitrogen (—N═) or —CR⁶═;
U³ is nitrogen (—NR⁵—) or —CR⁵═;
ring B is a 4-11 membered heterocycle;
each R³, if present, is independently selected from the group consisting of R⁸ and R⁹;
each R⁸ is independently selected from the group consisting of —OR⁹, —NR⁹R⁹, halogen, and —CN;
each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
each R¹⁰ is independently selected from the group consisting of —OR¹¹, —NR¹¹R¹¹ and halogen;
each R¹¹ is independently selected from the group consisting of C₁₋₆alkyl, C₃₋₁₀cycloalkyl, and 3-11 membered heterocyclyl; wherein the C₁₋₆alkyl, C₃₋₁₀cycloalkyl, and 3-11 membered heterocyclyl are all optionally substituted with one or more identical or different R¹²;
each R¹² is independently selected from the group consisting of halogen, C₁₋₆alkyl, C₁₋₆alkoxy, C₁₋₆haloalkyl;
q is selected from the group consisting of 0, 1, 2, and 3;
R⁵ is selected from the group consisting of C₆₋₁₀aryl, and 5-13 membered heteroaryl;
wherein the C₆₋₁₀aryl, and 5-13 membered heteroaryl are all optionally substituted with one or more, identical or different R¹³;
each R¹³ is independently selected from the group consisting of halogen, —CN, C₁₋₆alkyl, C₁₋₆alkenyl, C₁₋₆alkynyl, C₁₋₆haloalkyl, and C₃₋₁₀cycloalkyl, wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴;
each R¹⁴ is independently selected from the group consisting of halogen, —CN, C₁₋₆alkyl, C₁₋₆haloalkyl, and C₃₋₁₀cycloalkyl;
R⁶ is a selected from the group consisting of hydrogen, C₁₋₆alkyl, C₁₋₆haloalkyl, C₃₋₁₀cycloalkyl and 3-11 membered heterocyclyl; wherein C₁₋₆alkyl, C₁₋₆haloalkyl, C₃₋₁₀cycloalkyl and 3-11 membered heterocyclyl are all optionally substituted with one or more, identical or different R¹⁵;
each R¹⁵ is independently selected from the group consisting of halogen, C₁₋₆alkyl, and C₁₋₆haloalkyl;
or a salt thereof.

2. The compound according to claim 1 of the formula (II) or the salt thereof 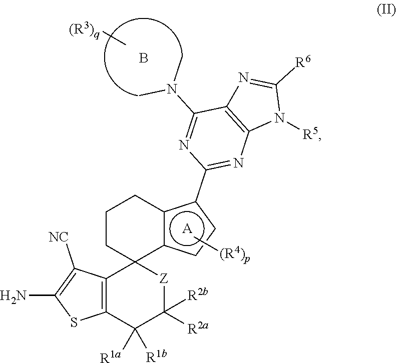
wherein
R^{1a}, R^{1b}, R^{2a}, R^{2b}, R³, R⁴, R⁵, R⁶ Z, ring A, ring B, p and q are as defined in claim 1.

3. The compound according to claim 1 of the formula (IIb) or the salt thereof 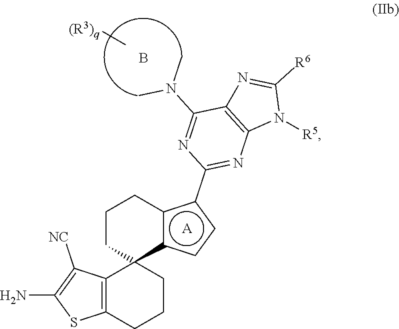
wherein
ring A, ring B, R³, R⁵, R⁶, and q are as defined in claim 1 and 2.

4. The compound or salt according to claim 1, wherein ring A is selected from the group consisting of

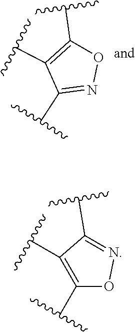

5. The compound or salt according to claim 1, wherein ring B is a 4-11 membered heterocycle.

6. The compound or salt according to claim 1, wherein each R³, if present, is independently selected from the group consisting of R⁸ and R⁹;
each R⁸ is independently selected from the group consisting of —OR⁹ and —NR⁹R⁹;
each R⁹ is independently selected from the group consisting of hydrogen, C₁₋₆alkyl, C₁₋₆alkoxy, C₁₋₆haloalkyl, C₃₋₁₀cycloalkyl, and 3-11 membered heterocyclyl, wherein the C₁₋₆alkyl, C₁₋₆alkoxy, C₁₋₆haloalkyl, C₃₋₁₀cycloalkyl and 3-11 membered heterocyclyl, are all optionally substituted with one or more, identical or different R¹⁰ and/or R¹¹;
each R¹⁰ is independently selected from the group consisting of —OR¹¹, —NR¹¹R¹¹ and halogen; each R¹¹ is independently selected from the group consisting of C₁₋₆alkyl, C₃₋₁₀cycloalkyl, and 3-11 membered heterocyclyl.

7. The compound or salt according to claim 5, wherein ring B—(R³)q is selected from the group consisting of

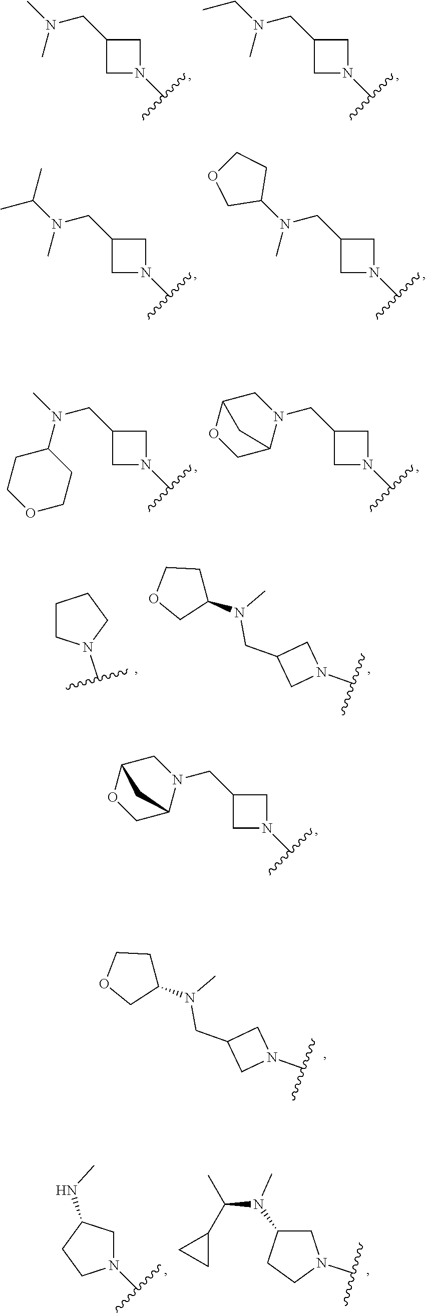

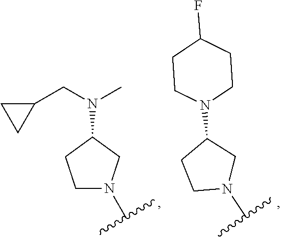 [SMILES: *N1CC[C@H](N(C)CC2CC2)C1 ; *N1CC[C@H](N2CCC(F)CC2)C1]

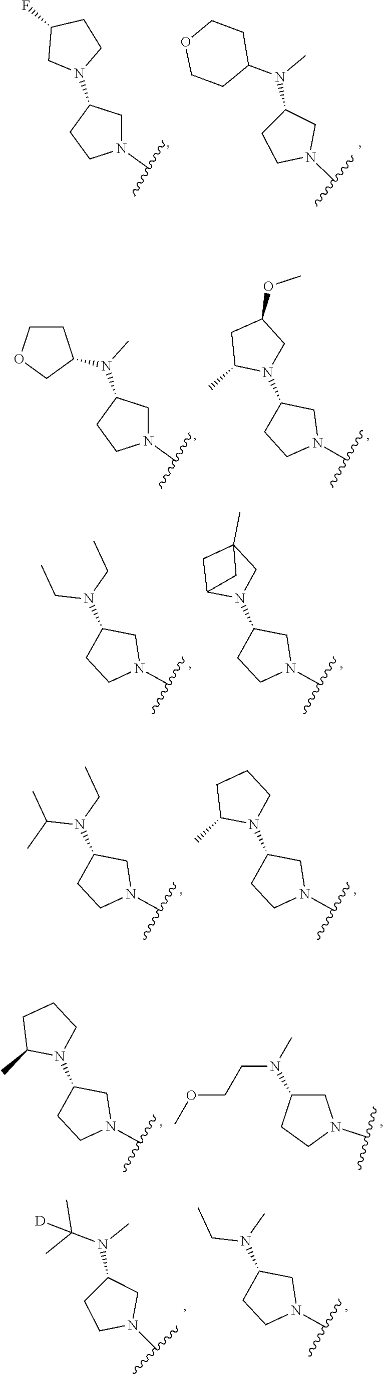

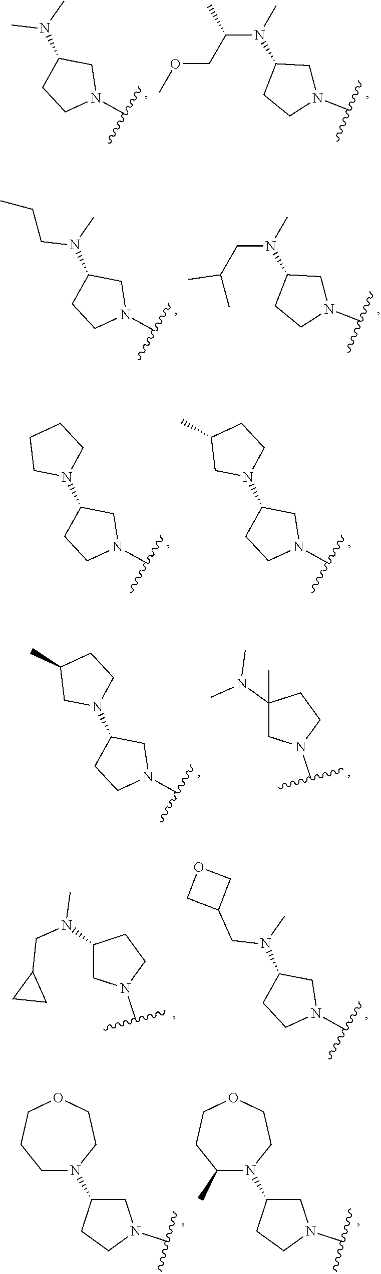

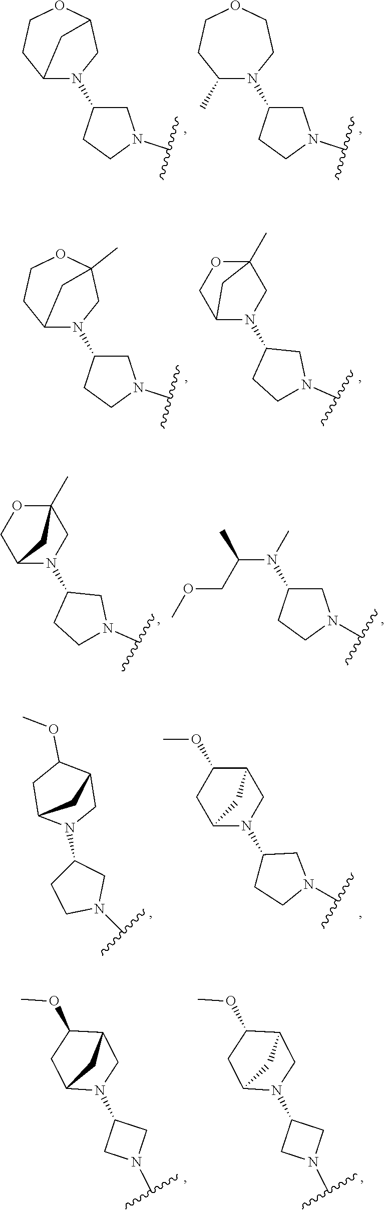

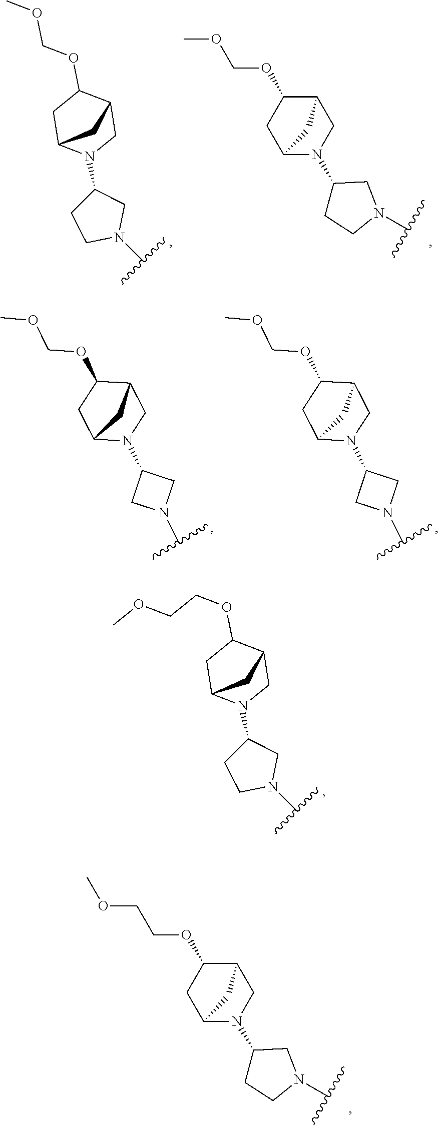

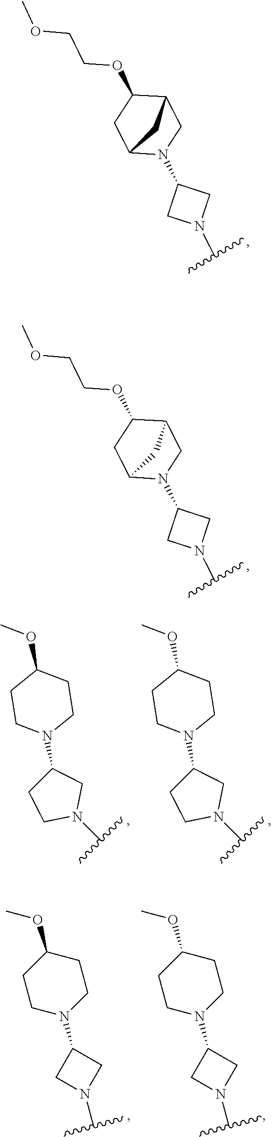

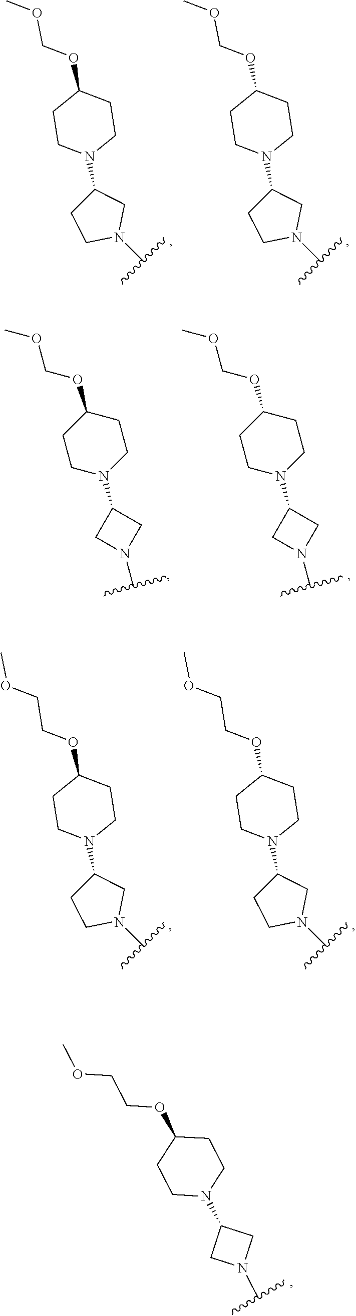

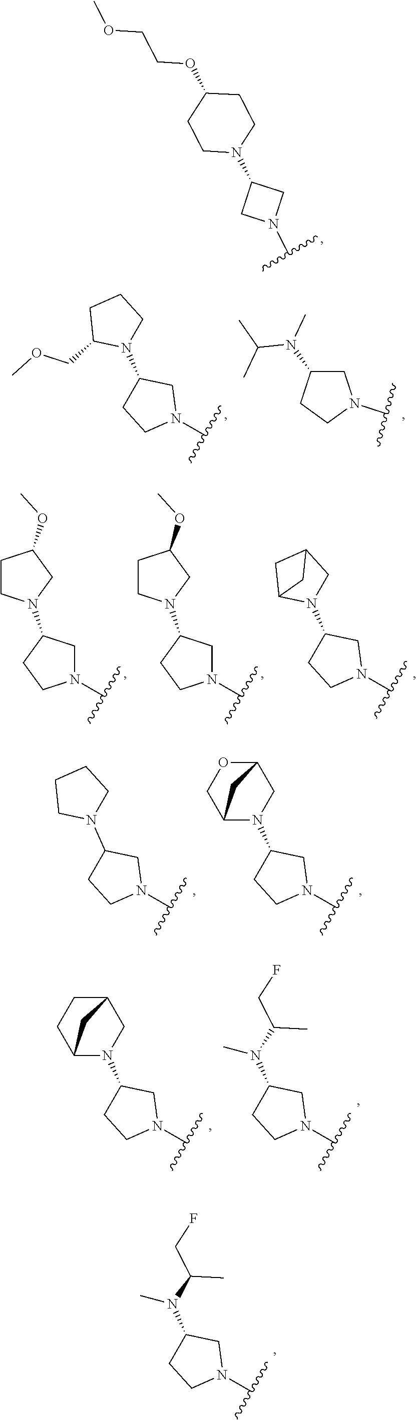

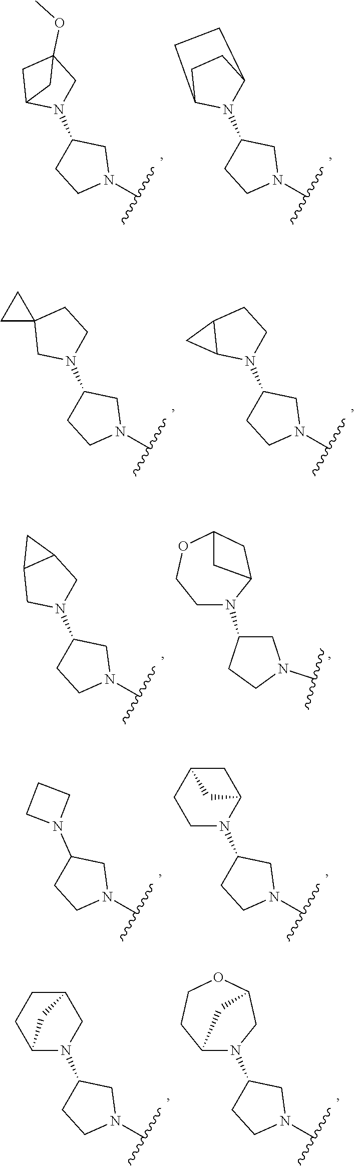

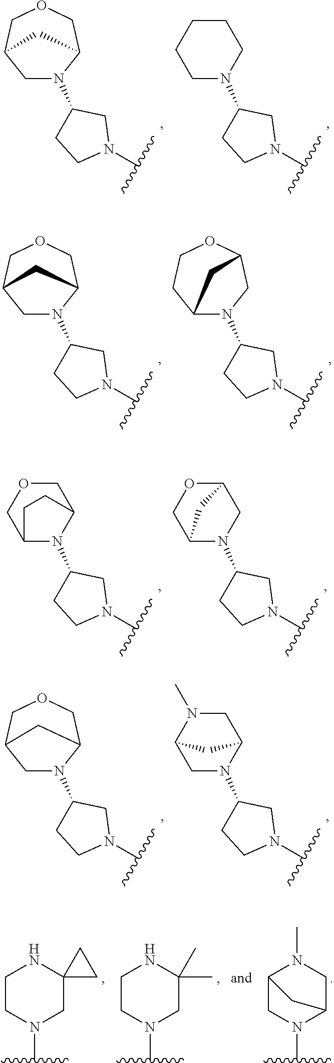

8. The compound or salt according to claim 1, wherein R⁵ is a 5-13 membered heteroaryl; wherein the 5-13 membered heteroaryl is optionally substituted with one or more, identical or different R¹³; each R¹³ is independently selected from the group consisting of halogen, —ON, C₁₋₆alkyl, C₁₋₆alkenyl,
C₁₋₆alkynyl, C₁₋₆haloalkyl, and C₃₋₁₀cycloalkyl; wherein the C₁₋₆alkyl, C₁₋₆alkenyl, C₁₋₆alkynyl, C₁₋₆haloalkyl, and C₃₋₁₀cycloalkyl are all optionally substituted with one or more identical or different R¹⁴; each R¹⁴ is independently selected from the group consisting of halogen, —ON, C₁₋₆alkyl, C₁₋₆haloalkyl, and C₃₋₁₀cycloalkyl.

9. The compound or salt according to claim 8, wherein R⁵ is selected from the group consisting of

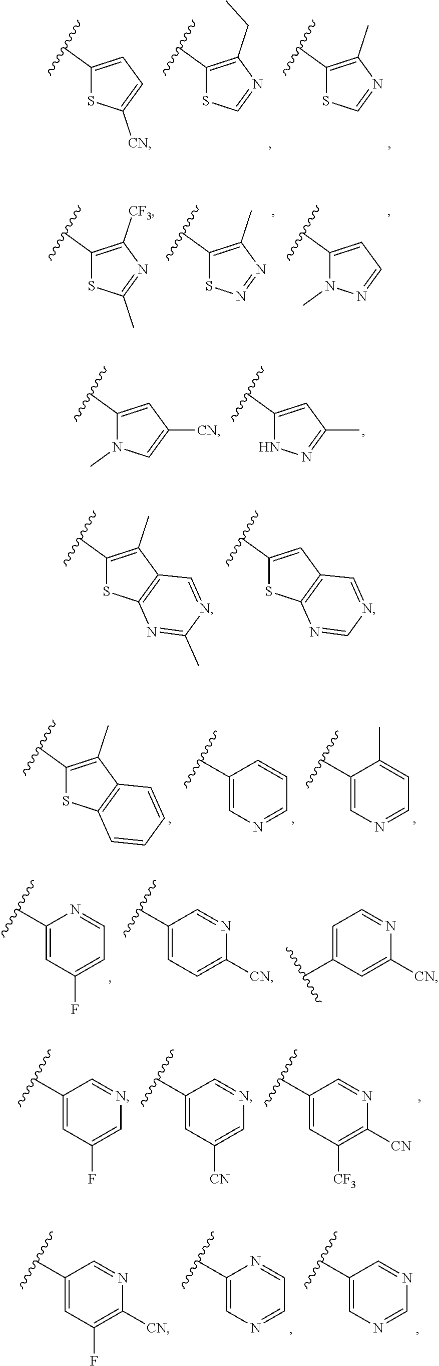

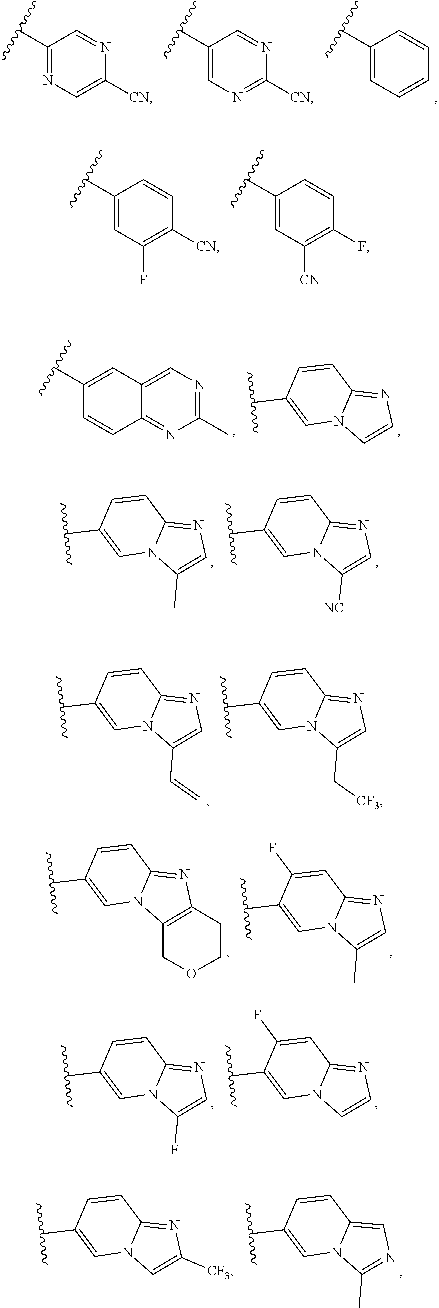

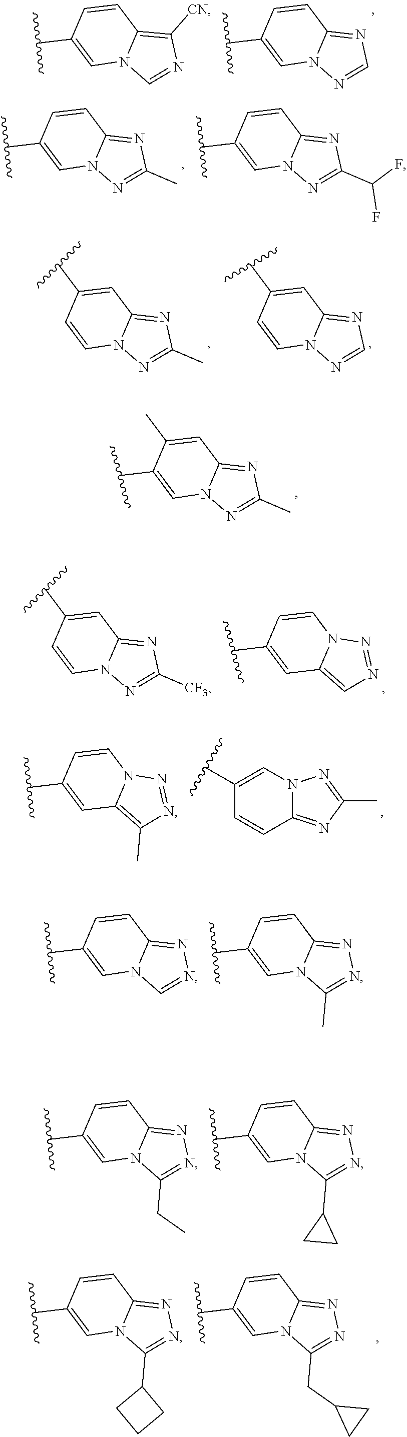

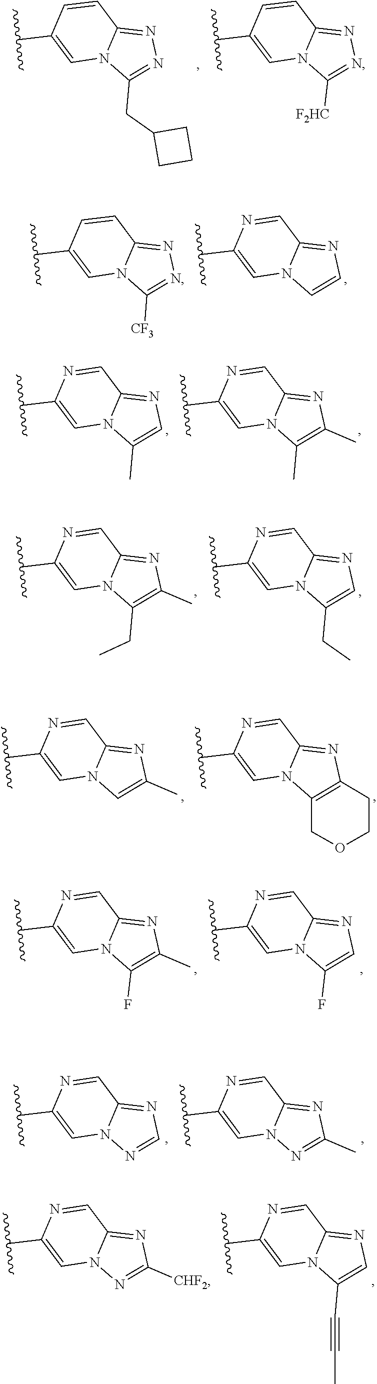

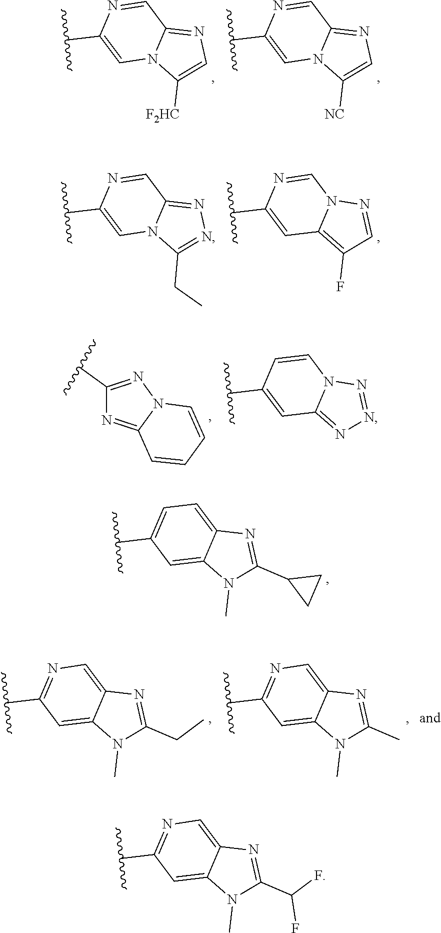

10. The compound or salt according to claim 1, wherein R⁶ is a selected from the group consisting of C₁₋₆alkyl, C₁₋₆haloalkyl, and C₃₋₁₀cycloalkyl; wherein
C₁₋₆alkyl, C₁₋₆haloalkyl, and C₃₋₁₀cycloalkyl are all optionally substituted with one or more, identical or different R¹⁵; each R¹⁵ is independently selected from the group consisting of halogen, C₁₋₆alkyl, and C₁₋₆haloalkyl.

11. The compound or salt according to claim 10, wherein R⁶ is selected from the group consisting of

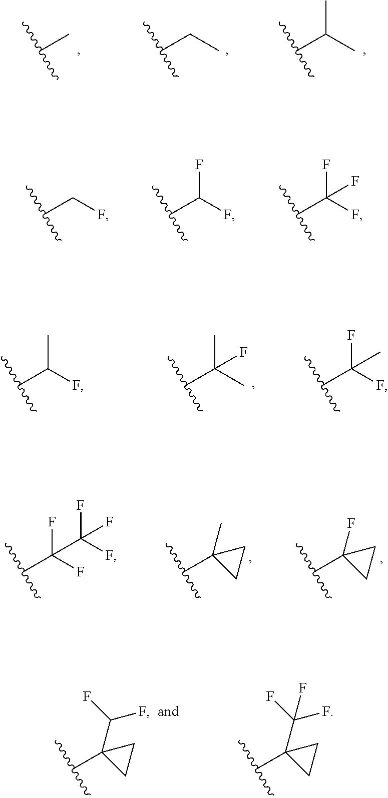

12. A compound selected from the group consisting of 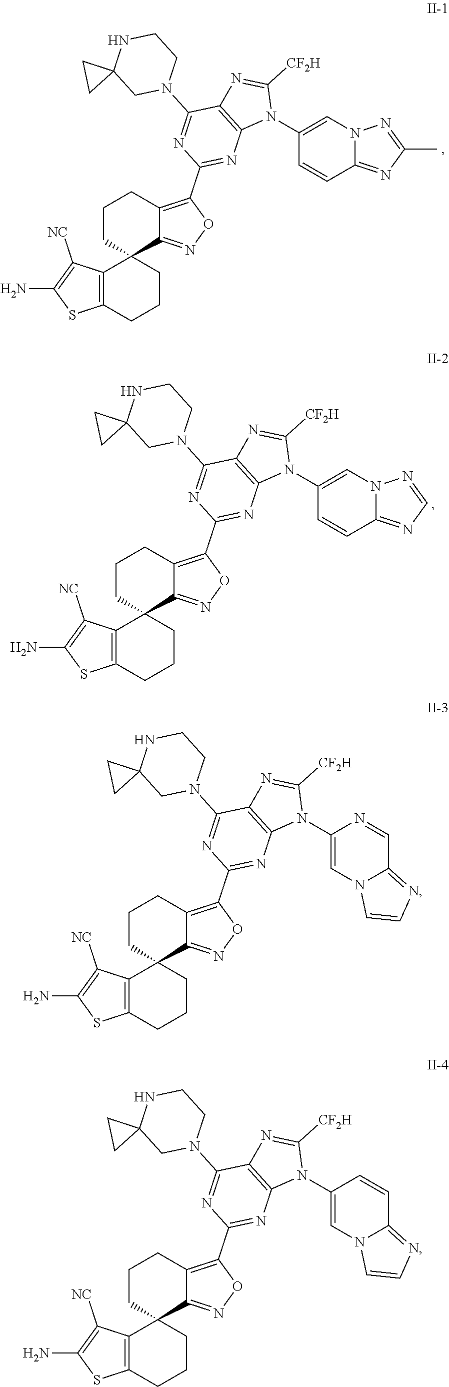 [SMILES: Cc1nc2ccc(-n3c(C(F)F)nc4c(N5CCNC6(CC6)C5)nc(-c5onc6c5CCC[C@@]65CCCc6sc(N)c(C#N)c65)nc43)cn2n1 ; N#Cc1c(N)sc2c1[C@@]1(CCC2)CCCc2c1noc2-c1nc(N2CCNC3(CC3)C2)c2nc(C(F)F)n(-c3ccc4ncnn4c3)c2n1 ; N#Cc1c(N)sc2c1[C@@]1(CCC2)CCCc2c1noc2-c1nc(N2CCNC3(CC3)C2)c2nc(C(F)F)n(-c3cn4ccnc4cn3)c2n1 ; N#Cc1c(N)sc2c1[C@@]1(CCC2)CCCc2c1noc2-c1nc(N2CCNC3(CC3)C2)c2nc(C(F)F)n(-c3ccc4nccn4c3)c2n1] 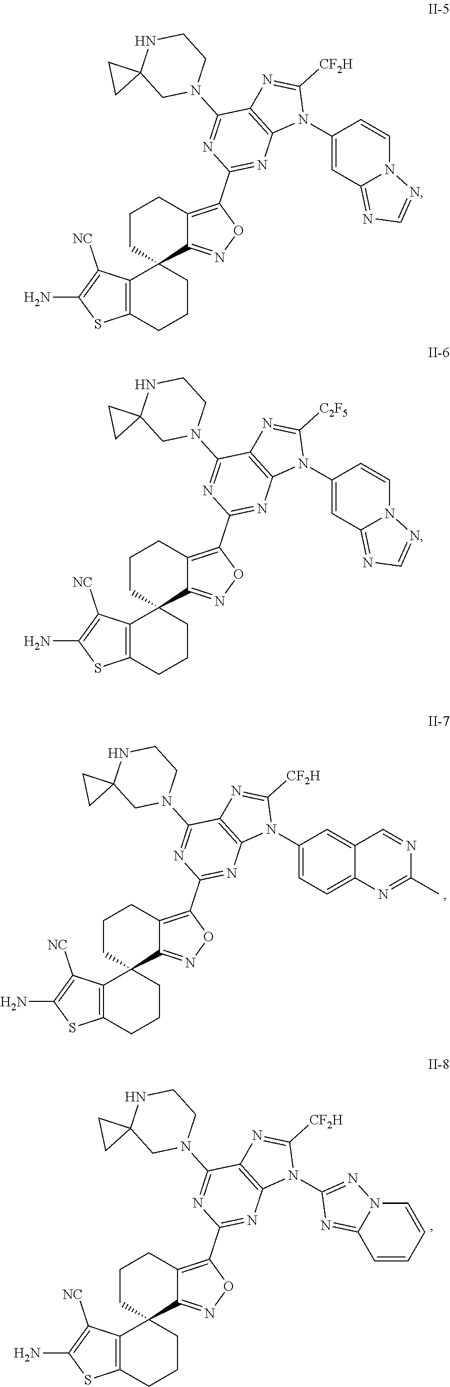 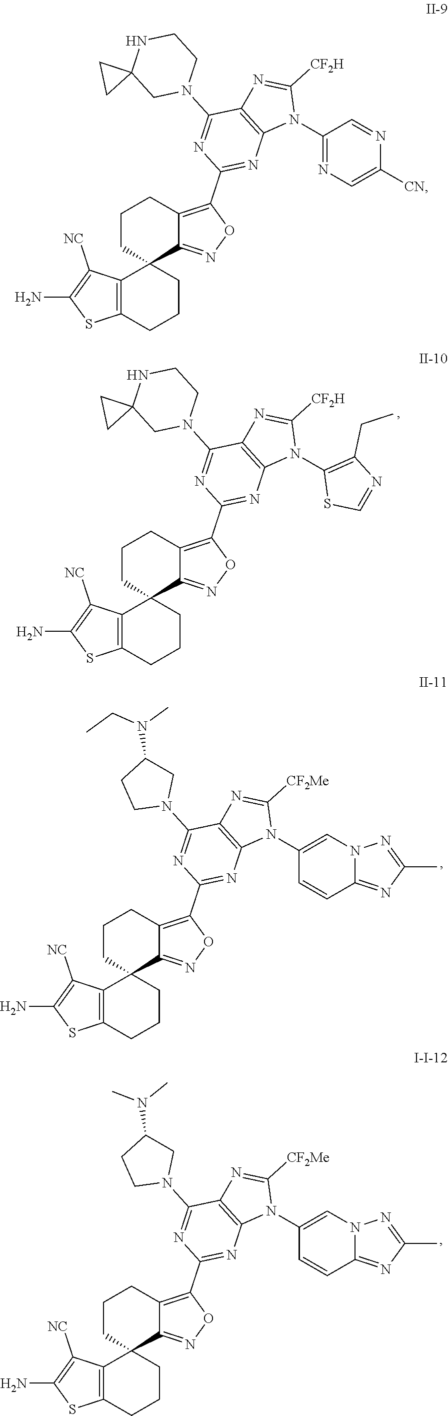 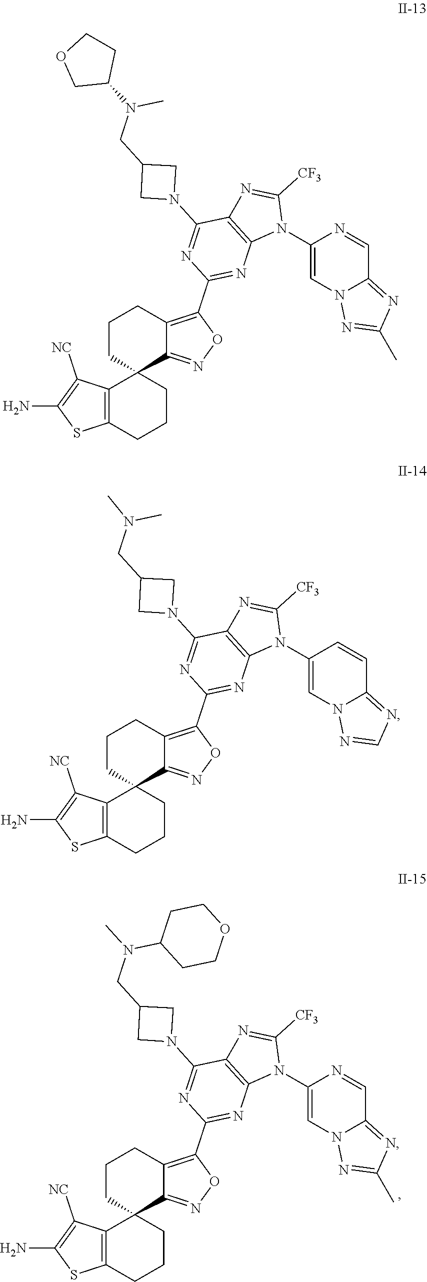 [SMILES: Cc1nc2cnc(-n3c(C(F)(F)F)nc4c(N5CC(CN(C)[C@H]6CCOC6)C5)nc(-c5onc6c5CCC[C@@]65CCCc6sc(N)c(C#N)c65)nc43)cn2n1 ; CN(C)CC1CN(c2nc(-c3onc4c3CCC[C@@]43CCCc4sc(N)c(C#N)c43)nc3c2nc(C(F)(F)F)n3-c2ccc3ncnn3c2)C1 ; Cc1nc2cnc(-n3c(C(F)(F)F)nc4c(N5CC(CN(C)C6CCOCC6)C5)nc(-c5onc6c5CCC[C@@]65CCCc6sc(N)c(C#N)c65)nc43)cn2n1] 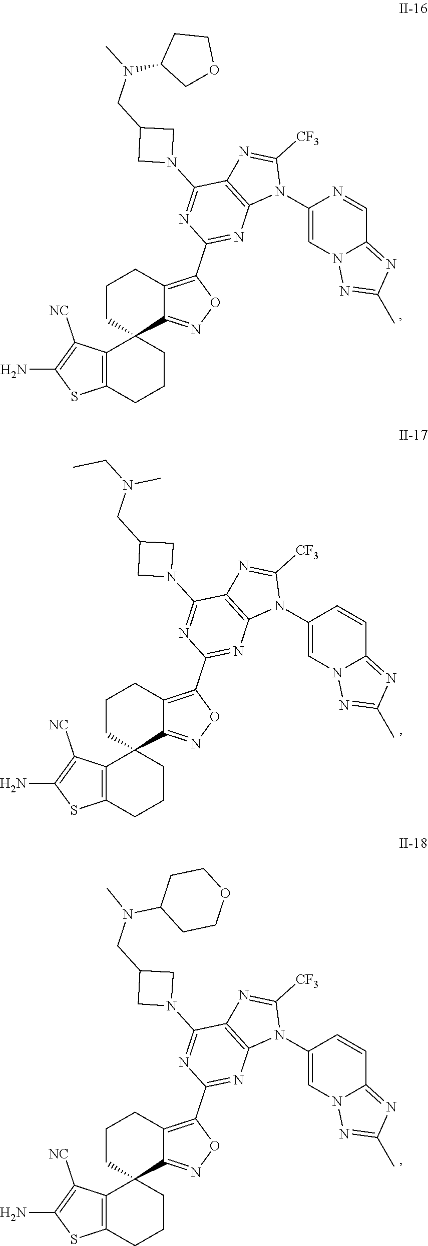 [SMILES: Cc1nc2cnc(-n3c(C(F)(F)F)nc4c(N5CC(CN(C)[C@@H]6CCOC6)C5)nc(-c5onc6c5CCC[C@@]65CCCc6sc(N)c(C#N)c65)nc43)cn2n1 ; CCN(C)CC1CN(c2nc(-c3onc4c3CCC[C@@]43CCCc4sc(N)c(C#N)c43)nc3c2nc(C(F)(F)F)n3-c2ccc3nc(C)nn3c2)C1 ; Cc1nc2ccc(-n3c(C(F)(F)F)nc4c(N5CC(CN(C)C6CCOCC6)C5)nc(-c5onc6c5CCC[C@@]65CCCc6sc(N)c(C#N)c65)nc43)cn2n1] 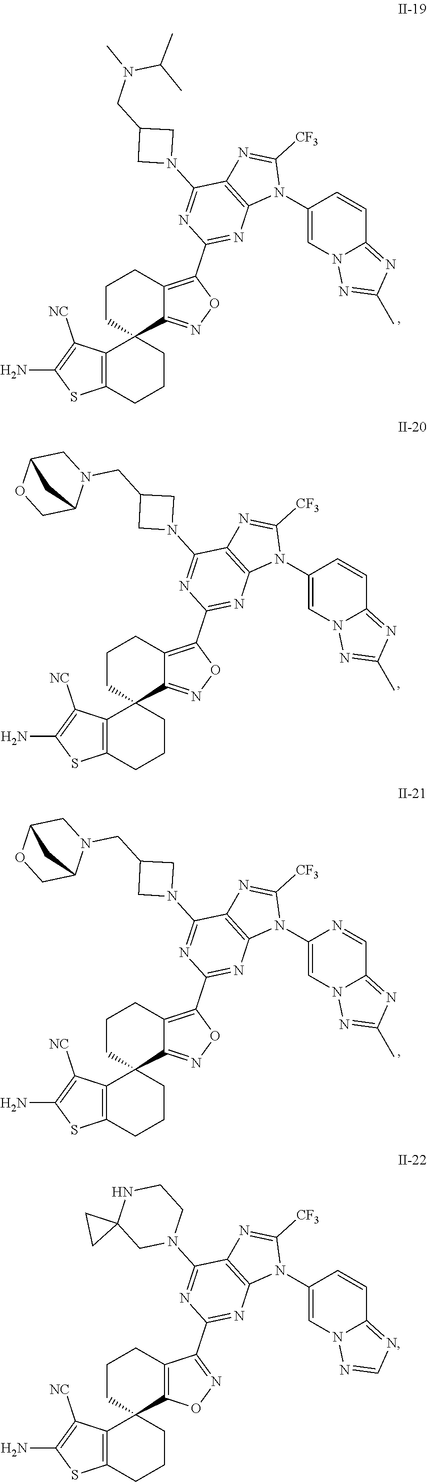 [SMILES: Cc1nc2ccc(-n3c(C(F)(F)F)nc4c(N5CC(CN(C)C(C)C)C5)nc(-c5onc6c5CCC[C@@]65CCCc6sc(N)c(C#N)c65)nc43)cn2n1 ; Cc1nc2ccc(-n3c(C(F)(F)F)nc4c(N5CC(CN6C[C@@H]7C[C@H]6CO7)C5)nc(-c5onc6c5CCC[C@@]65CCCc6sc(N)c(C#N)c65)nc43)cn2n1 ; Cc1nc2cnc(-n3c(C(F)(F)F)nc4c(N5CC(CN6C[C@@H]7C[C@H]6CO7)C5)nc(-c5onc6c5CCC[C@@]65CCCc6sc(N)c(C#N)c65)nc43)cn2n1 ; N#Cc1c(N)sc2c1[C@@]1(CCC2)CCCc2c(-c3nc(N4CCNC5(CC5)C4)c4nc(C(F)(F)F)n(-c5ccc6ncnn6c5)c4n3)noc21] 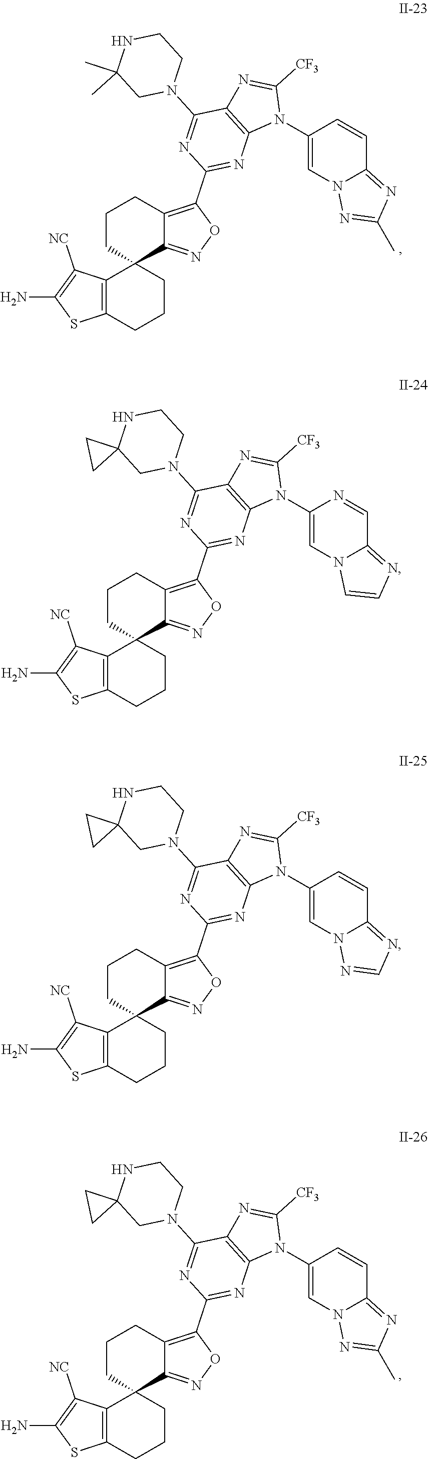 [SMILES: Cc1nc2ccc(-n3c(C(F)(F)F)nc4c(N5CCNC(C)(C)C5)nc(-c5onc6c5CCC[C@@]65CCCc6sc(N)c(C#N)c65)nc43)cn2n1 ; N#Cc1c(N)sc2c1[C@@]1(CCC2)CCCc2c1noc2-c1nc(N2CCNC3(CC3)C2)c2nc(C(F)(F)F)n(-c3cn4ccnc4cn3)c2n1 ; N#Cc1c(N)sc2c1[C@@]1(CCC2)CCCc2c1noc2-c1nc(N2CCNC3(CC3)C2)c2nc(C(F)(F)F)n(-c3ccc4ncnn4c3)c2n1 ; Cc1nc2ccc(-n3c(C(F)(F)F)nc4c(N5CCNC6(CC6)C5)nc(-c5onc6c5CCC[C@@]65CCCc6sc(N)c(C#N)c65)nc43)cn2n1] 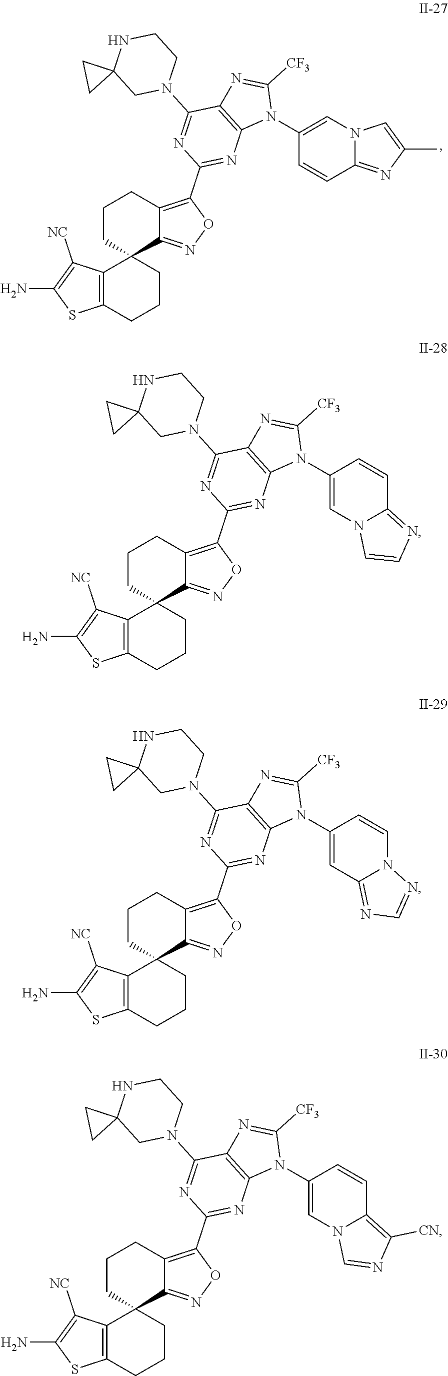 [SMILES: Cc1cn2cc(-n3c(C(F)(F)F)nc4c(N5CCNC6(CC6)C5)nc(-c5onc6c5CCC[C@@]65CCCc6sc(N)c(C#N)c65)nc43)ccc2n1 ; N#Cc1c(N)sc2c1[C@@]1(CCC2)CCCc2c1noc2-c1nc(N2CCNC3(CC3)C2)c2nc(C(F)(F)F)n(-c3ccc4nccn4c3)c2n1 ; N#Cc1c(N)sc2c1[C@@]1(CCC2)CCCc2c1noc2-c1nc(N2CCNC3(CC3)C2)c2nc(C(F)(F)F)n(-c3ccn4ncnc4c3)c2n1 ; N#Cc1c(N)sc2c1[C@@]1(CCC2)CCCc2c1noc2-c1nc(N2CCNC3(CC3)C2)c2nc(C(F)(F)F)n(-c3ccc4c(C#N)ncn4c3)c2n1] 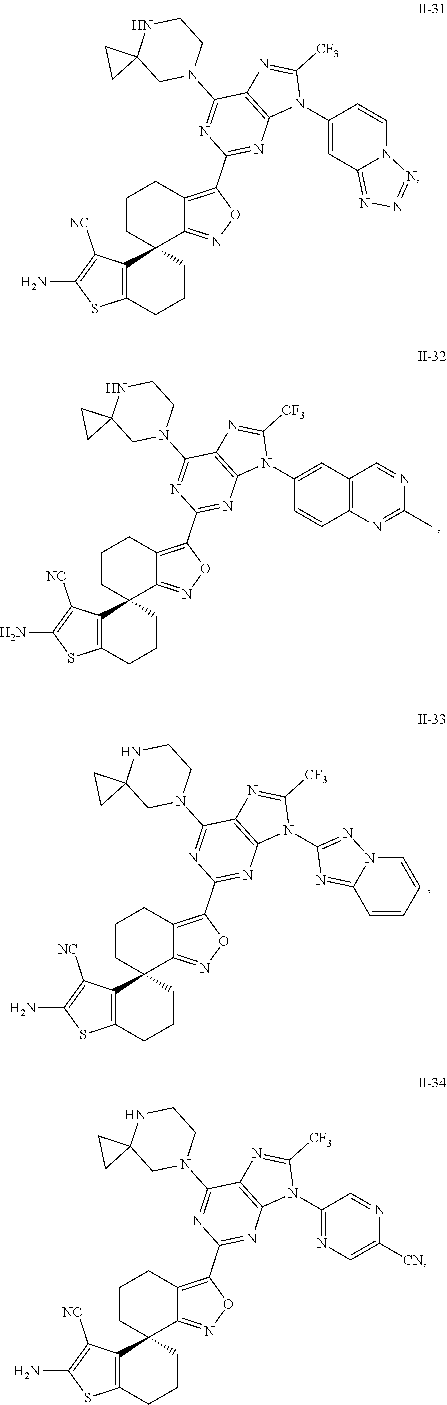 [SMILES: N#Cc1c(N)sc2c1[C@@]1(CCC2)CCCc2c1noc2-c1nc(N2CCNC3(CC3)C2)c2nc(C(F)(F)F)n(-c3ccn4nnnc4c3)c2n1 ; Cc1ncc2cc(-n3c(C(F)(F)F)nc4c(N5CCNC6(CC6)C5)nc(-c5onc6c5CCC[C@@]65CCCc6sc(N)c(C#N)c65)nc43)ccc2n1 ; N#Cc1c(N)sc2c1[C@@]1(CCC2)CCCc2c1noc2-c1nc(N2CCNC3(CC3)C2)c2nc(C(F)(F)F)n(-c3nc4ccccn4n3)c2n1 ; N#Cc1cnc(-n2c(C(F)(F)F)nc3c(N4CCNC5(CC5)C4)nc(-c4onc5c4CCC[C@@]54CCCc5sc(N)c(C#N)c54)nc32)cn1] 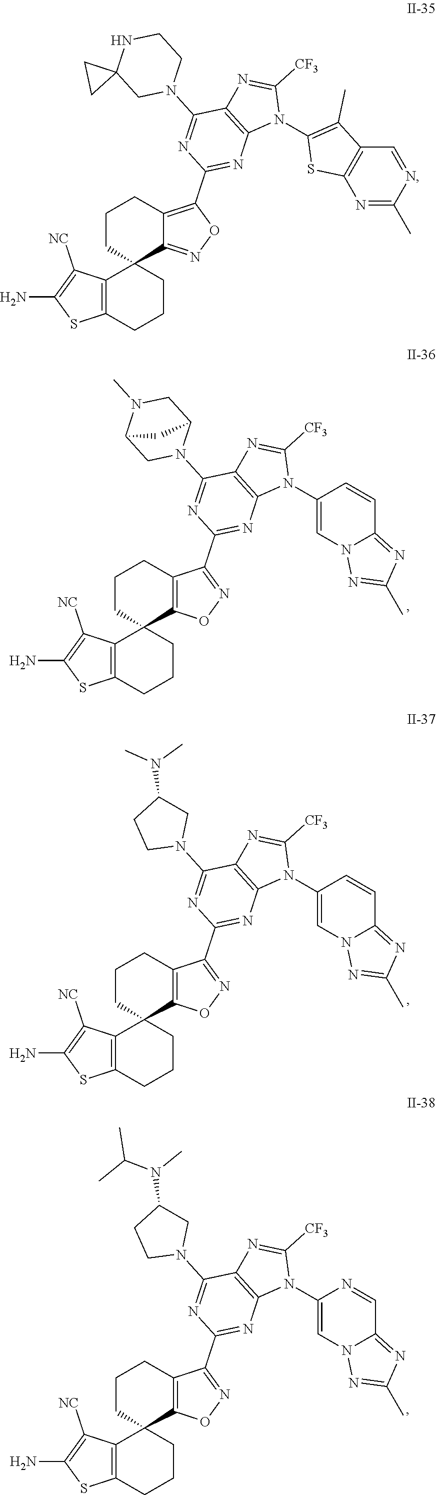 [SMILES: Cc1ncc2c(C)c(-n3c(C(F)(F)F)nc4c(N5CCNC6(CC6)C5)nc(-c5onc6c5CCC[C@@]65CCCc6sc(N)c(C#N)c65)nc43)sc2n1 ; Cc1nc2ccc(-n3c(C(F)(F)F)nc4c(N5C[C@@H]6C[C@H]5CN6C)nc(-c5noc6c5CCC[C@@]65CCCc6sc(N)c(C#N)c65)nc43)cn2n1 ; Cc1nc2ccc(-n3c(C(F)(F)F)nc4c(N5CC[C@H](N(C)C)C5)nc(-c5noc6c5CCC[C@@]65CCCc6sc(N)c(C#N)c65)nc43)cn2n1 ; Cc1nc2cnc(-n3c(C(F)(F)F)nc4c(N5CC[C@H](N(C)C(C)C)C5)nc(-c5noc6c5CCC[C@@]65CCCc6sc(N)c(C#N)c65)nc43)cn2n1] 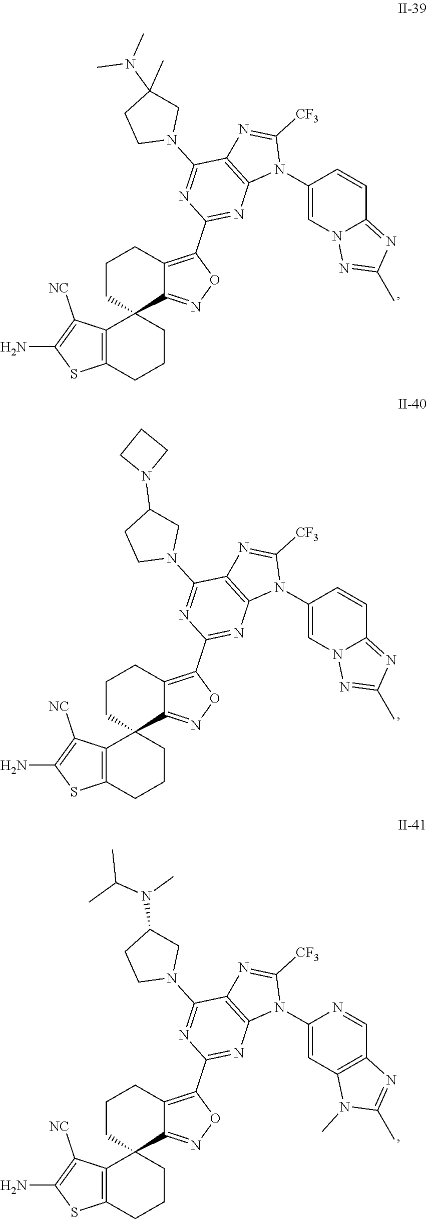 [SMILES: Cc1nc2ccc(-n3c(C(F)(F)F)nc4c(N5CCC(C)(N(C)C)C5)nc(-c5onc6c5CCC[C@@]65CCCc6sc(N)c(C#N)c65)nc43)cn2n1 ; Cc1nc2ccc(-n3c(C(F)(F)F)nc4c(N5CCC(N6CCC6)C5)nc(-c5onc6c5CCC[C@@]65CCCc6sc(N)c(C#N)c65)nc43)cn2n1 ; Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C] 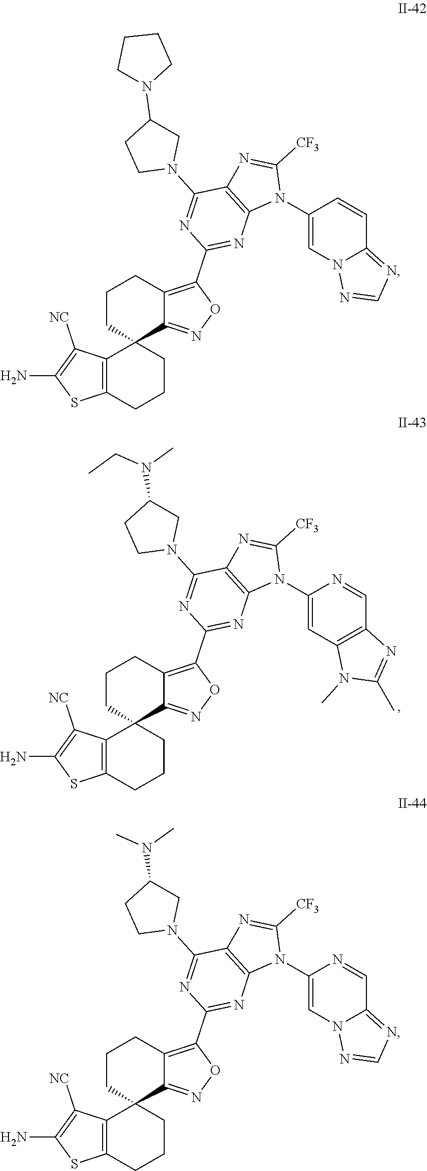 [SMILES: N#Cc1c(N)sc2c1[C@@]1(CCC2)CCCc2c1noc2-c1nc(N2CCC(N3CCCC3)C2)c2nc(C(F)(F)F)n(-c3ccc4ncnn4c3)c2n1 ; CCN(C)[C@H]1CCN(c2nc(-c3onc4c3CCC[C@@]43CCCc4sc(N)c(C#N)c43)nc3c2nc(C(F)(F)F)n3-c2cc3c(cn2)nc(C)n3C)C1 ; CN(C)[C@H]1CCN(c2nc(-c3onc4c3CCC[C@@]43CCCc4sc(N)c(C#N)c43)nc3c2nc(C(F)(F)F)n3-c2cn3ncnc3cn2)C1] 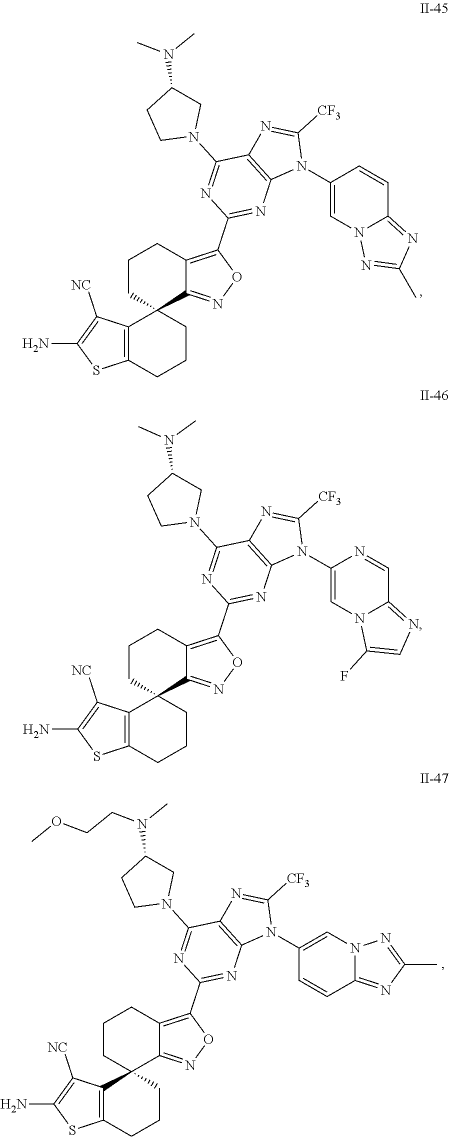 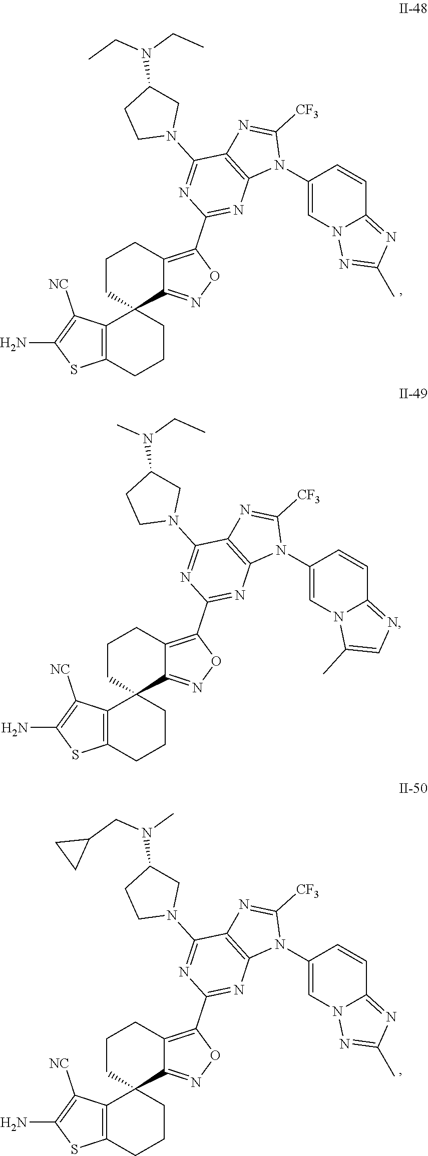 [SMILES: CCN(CC)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1 ; CCN(C)[C@H]1CCN(c2nc(-c3onc4c3CCC[C@@]43CCCc4sc(N)c(C#N)c43)nc3c2nc(C(F)(F)F)n3-c2ccc3ncc(C)n3c2)C1 ; Cc1nc2ccc(-n3c(C(F)(F)F)nc4c(N5CC[C@H](N(C)CC6CC6)C5)nc(-c5onc6c5CCC[C@@]65CCCc6sc(N)c(C#N)c65)nc43)cn2n1] 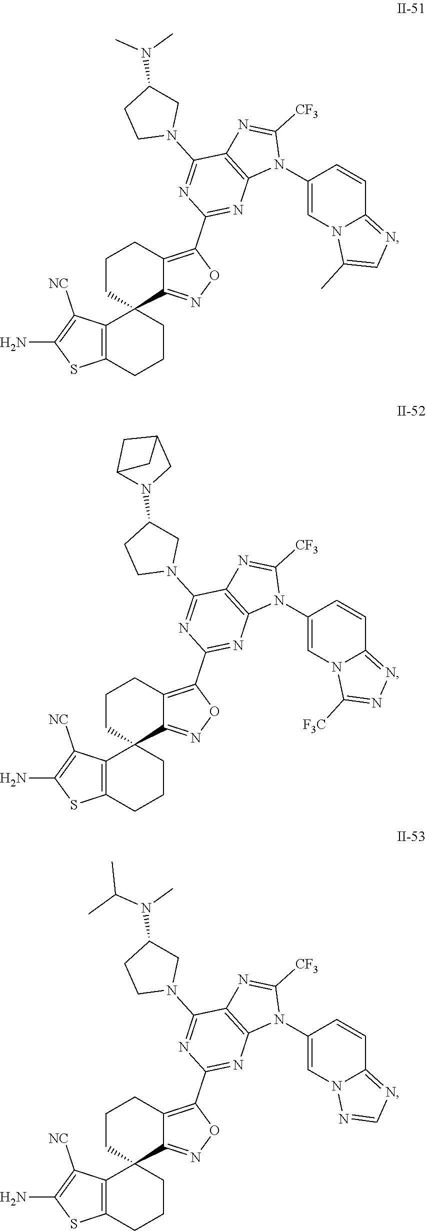 [SMILES: Cc1cnc2ccc(-n3c(C(F)(F)F)nc4c(N5CC[C@H](N(C)C)C5)nc(-c5onc6c5CCC[C@@]65CCCc6sc(N)c(C#N)c65)nc43)cn12 ; N#Cc1c(N)sc2c1[C@@]1(CCC2)CCCc2c1noc2-c1nc(N2CC[C@H](N3CC4CC3C4)C2)c2nc(C(F)(F)F)n(-c3ccc4nnc(C(F)(F)F)n4c3)c2n1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1] 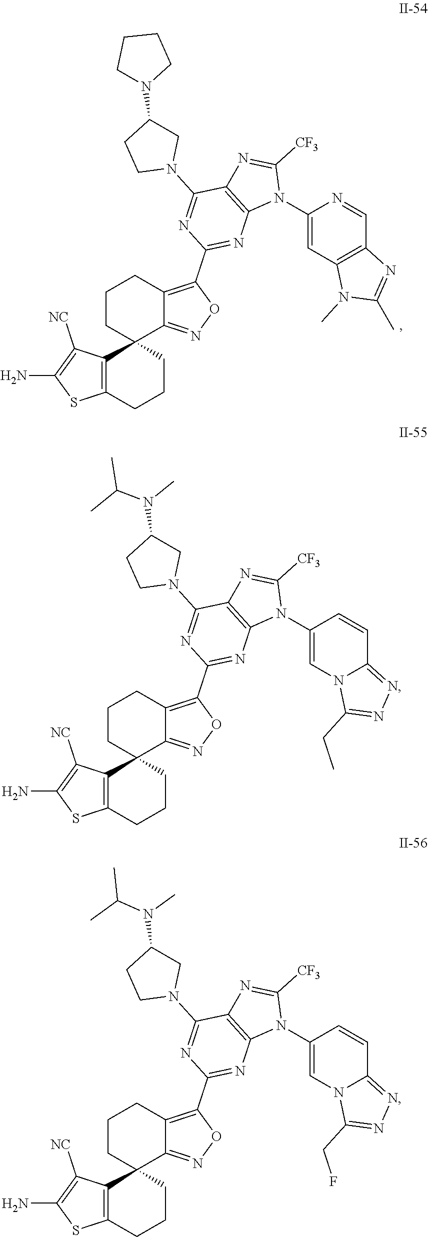 [SMILES: Cc1nc2cnc(-n3c(C(F)(F)F)nc4c(N5CC[C@H](N6CCCC6)C5)nc(-c5onc6c5CCC[C@@]65CCCc6sc(N)c(C#N)c65)nc43)cc2n1C ; CCc1nnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12 ; CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(CF)n3c2)C1] 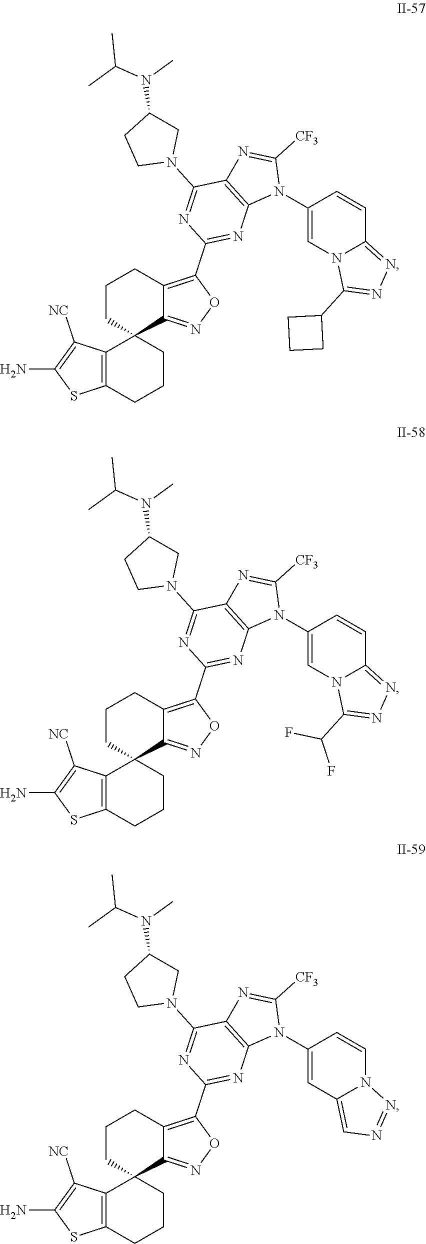 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C4CCC4)n3c2)C1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C(F)F)n3c2)C1 ; CC(C)N(C)[C@H]1CCN(c2nc(-c3onc4c3CCC[C@@]43CCCc4sc(N)c(C#N)c43)nc3c2nc(C(F)(F)F)n3-c2ccn3nncc3c2)C1] 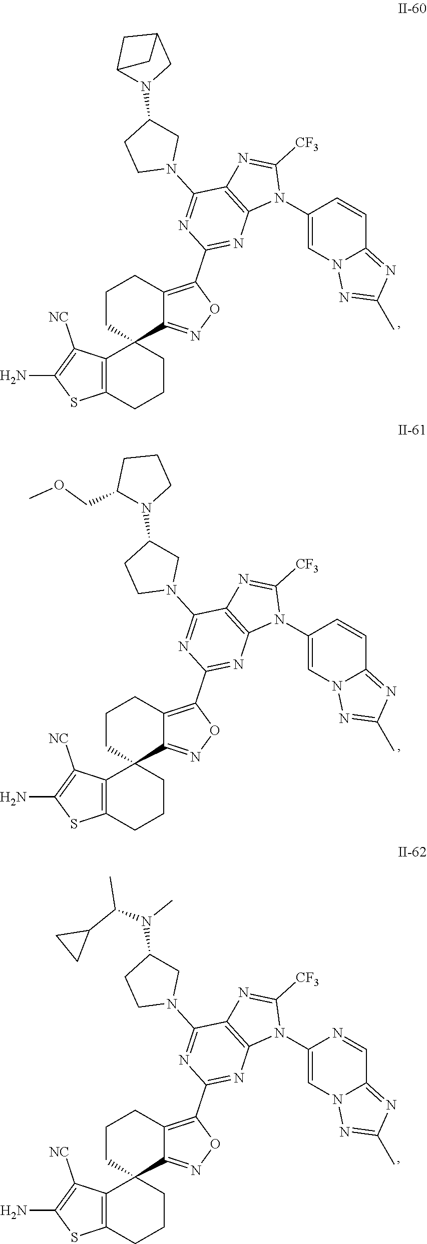 [SMILES: Cc1nc2ccc(-n3c(C(F)(F)F)nc4c(N5CC[C@H](N6CC7CC6C7)C5)nc(-c5onc6c5CCC[C@@]65CCCc6sc(N)c(C#N)c65)nc43)cn2n1 ; COC[C@@H]1CCCN1[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1 ; Cc1nc2cnc(-n3c(C(F)(F)F)nc4c(N5CC[C@H](N(C)[C@@H](C)C6CC6)C5)nc(-c5onc6c5CCC[C@@]65CCCc6sc(N)c(C#N)c65)nc43)cn2n1] 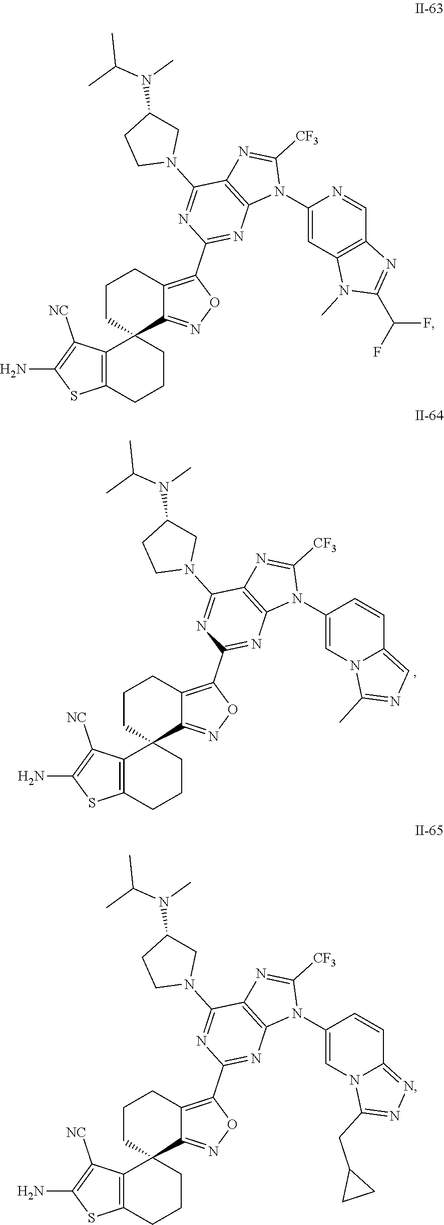 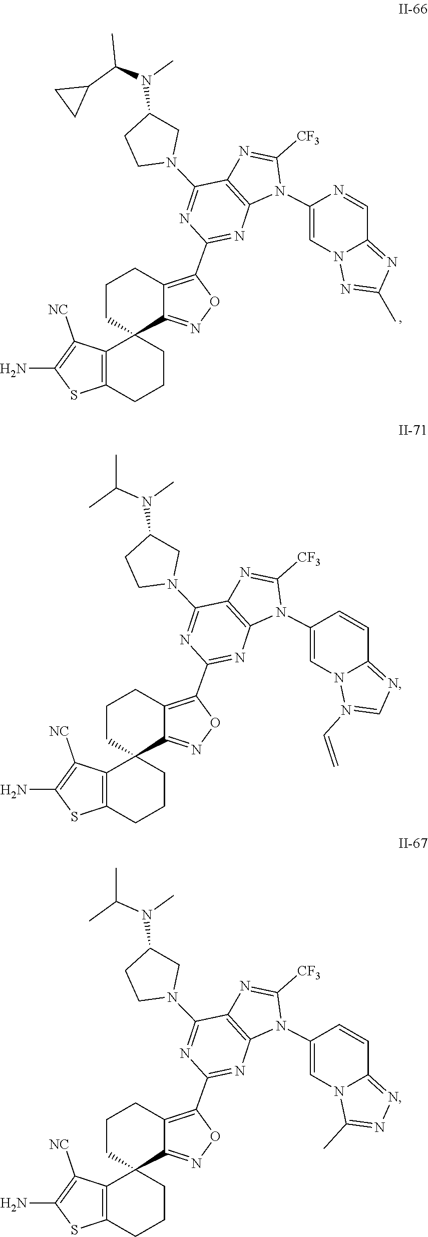 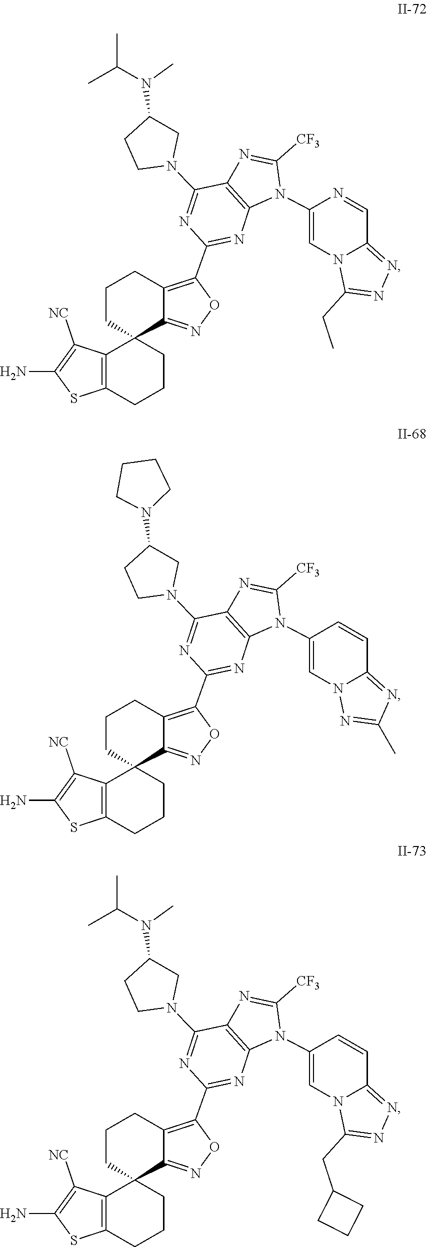 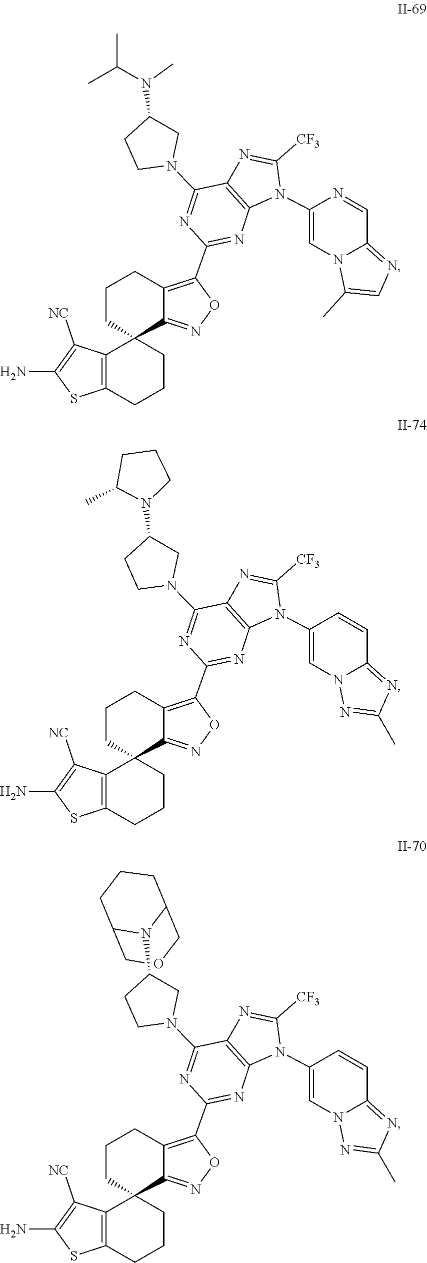 [SMILES: Cc1cnc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn12 ; Cc1nc2ccc(-n3c(C(F)(F)F)nc4c(N5CC[C@H](N6CCC[C@H]6C)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N6C7CCCC6COC7)C5)nc(-c5onc6c5CCC[C@@]65CCCc6sc(N)c(C#N)c65)nc43)cn2n1] 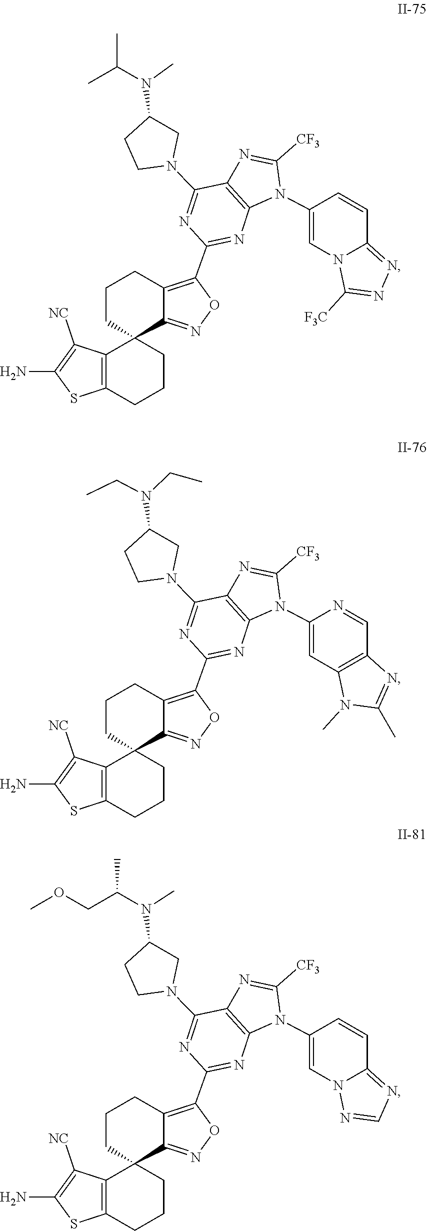 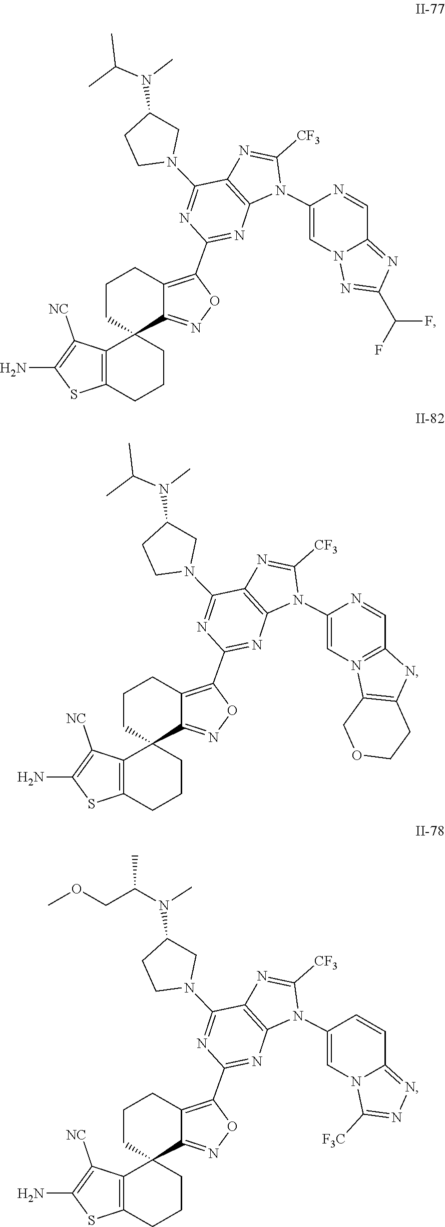 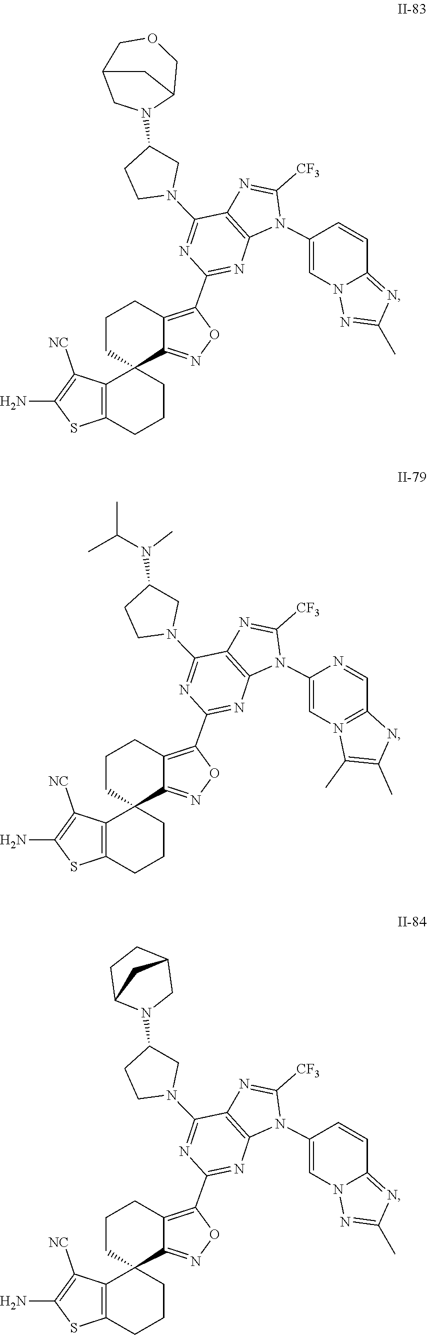 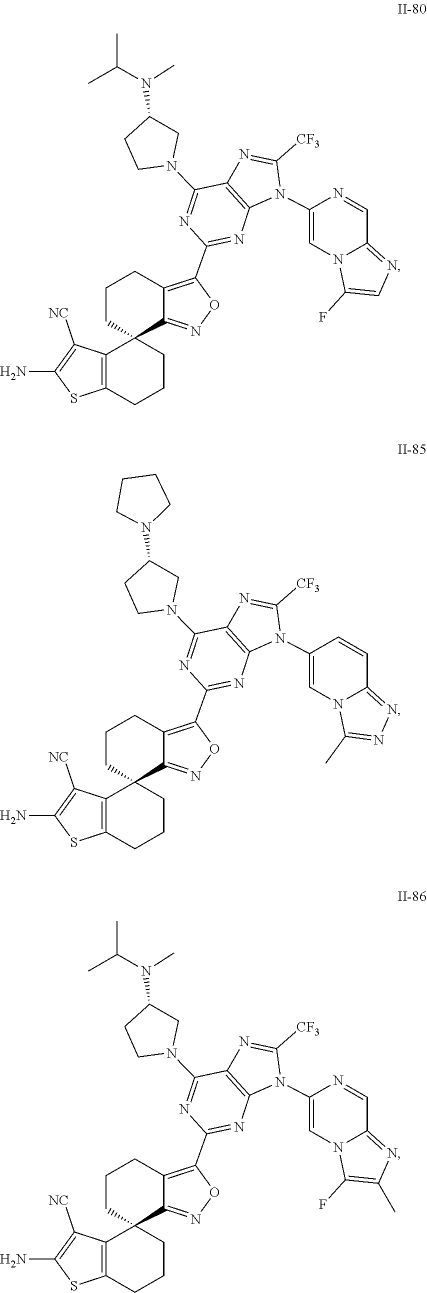 [SMILES: CC(C)N(C)[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1 ; Cc1nnc2ccc(-n3c(C(F)(F)F)nc4c(N5CC[C@H](N6CCCC6)C5)nc(-c5onc6c5CCC[C@@]65CCCc6sc(N)c(C#N)c65)nc43)cn12 ; Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1F] 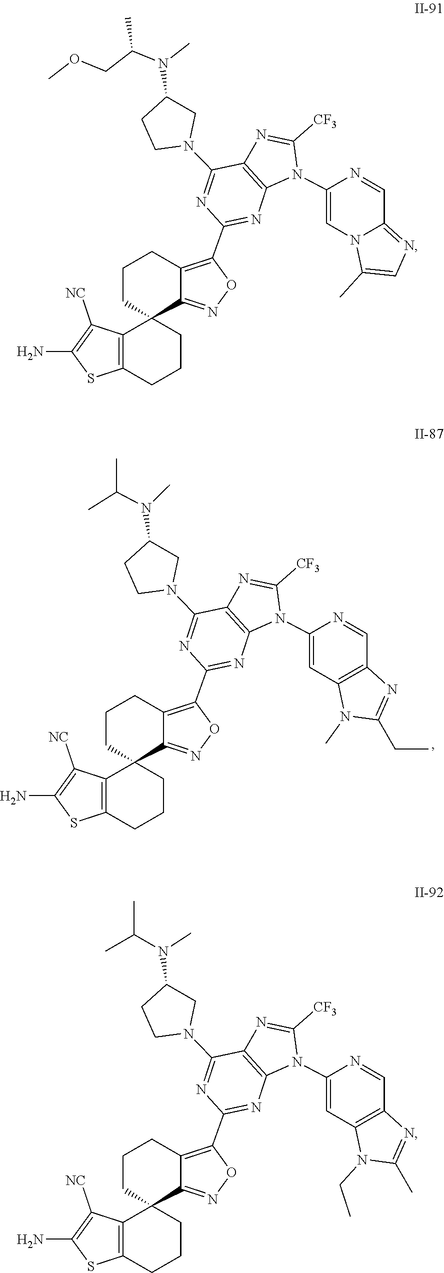 [SMILES: COC[C@H](C)N(C)[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1 ; CCc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C ; CCn1c(C)nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc21] 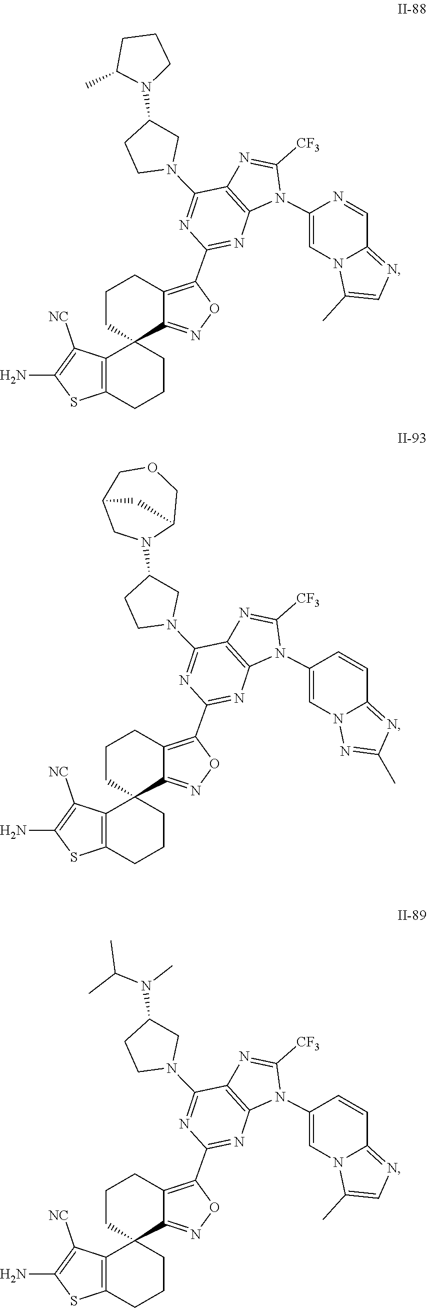 [SMILES: Cc1cnc2cnc(-n3c(C(F)(F)F)nc4c(N5CC[C@H](N6CCC[C@H]6C)C5)nc(-c5onc6c5CCC[C@@]65CCCc6sc(N)c(C#N)c65)nc43)cn12 ; Cc1nc2ccc(-n3c(C(F)(F)F)nc4c(N5CC[C@H](N6C[C@H]7COC[C@@H]6C7)C5)nc(-c5onc6c5CCC[C@@]65CCCc6sc(N)c(C#N)c65)nc43)cn2n1 ; Cc1cnc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn12] 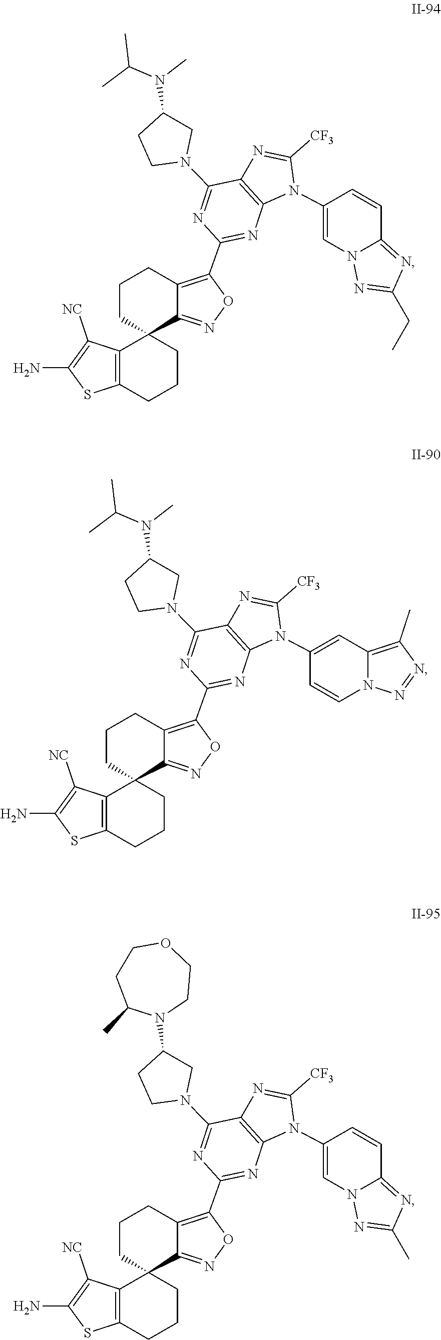 [SMILES: CCc1nc2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1 ; Cc1nnn2ccc(-n3c(C(F)(F)F)nc4c(N5CC[C@H](N(C)C(C)C)C5)nc(-c5onc6c5CCC[C@@]65CCCc6sc(N)c(C#N)c65)nc43)cc12 ; Cc1nc2ccc(-n3c(C(F)(F)F)nc4c(N5CC[C@H](N6CCOCC[C@@H]6C)C5)nc(-c5onc6c5CCC[C@@]65CCCc6sc(N)c(C#N)c65)nc43)cn2n1] 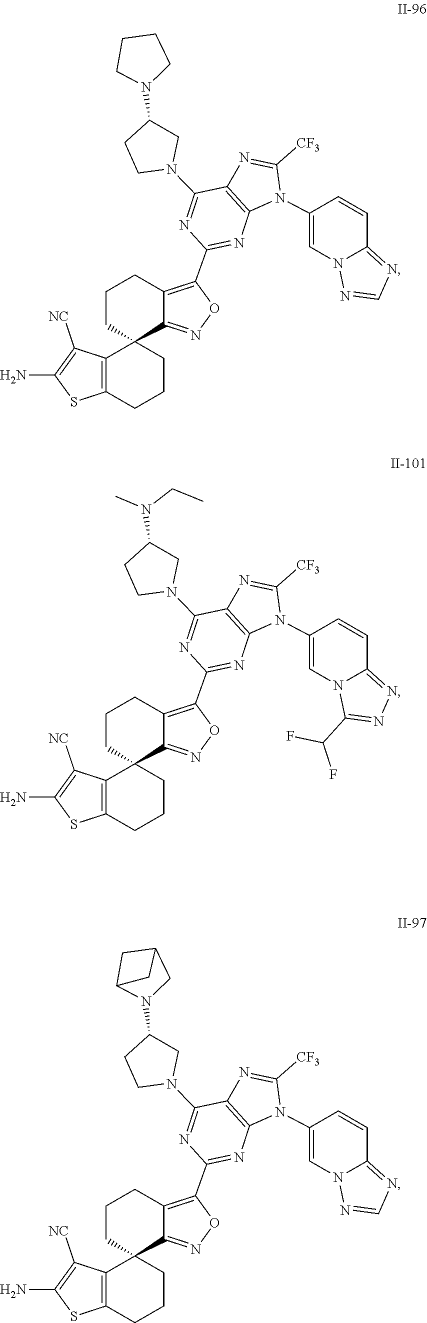 [SMILES: N#Cc1c(N)sc2c1[C@@]1(CCC2)CCCc2c1noc2-c1nc(N2CC[C@H](N3CCCC3)C2)c2nc(C(F)(F)F)n(-c3ccc4ncnn4c3)c2n1 ; CCN(C)[C@H]1CCN(c2nc(-c3onc4c3CCC[C@@]43CCCc4sc(N)c(C#N)c43)nc3c2nc(C(F)(F)F)n3-c2ccc3nnc(C(F)F)n3c2)C1 ; N#Cc1c(N)sc2c1[C@@]1(CCC2)CCCc2c1noc2-c1nc(N2CC[C@H](N3CC4CC3C4)C2)c2nc(C(F)(F)F)n(-c3ccc4ncnn4c3)c2n1] 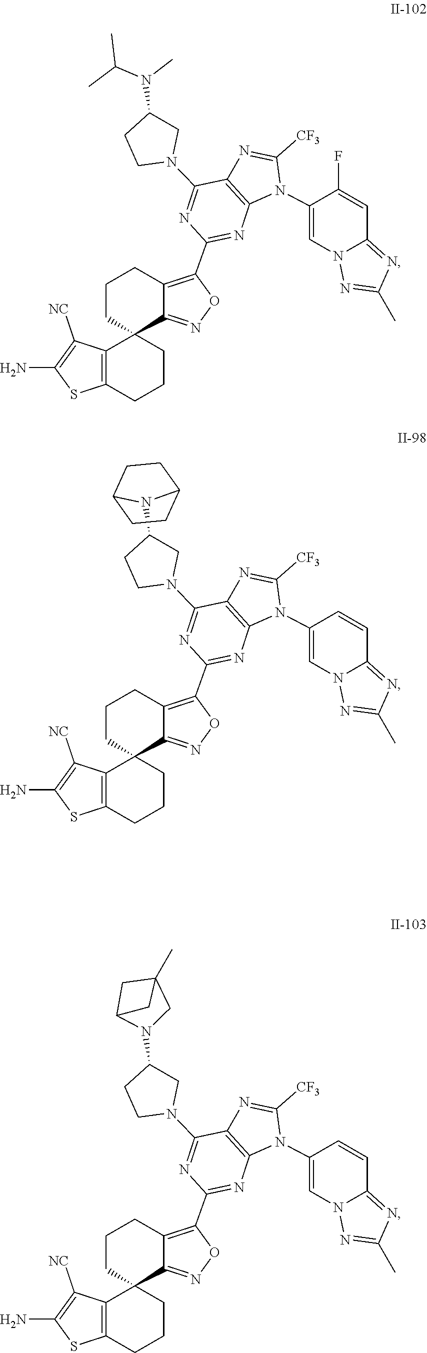 [SMILES: Cc1nc2cc(F)c(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N6C7CCC6CC7)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N6CC7(C)CC6C7)C5)nc(-c5onc6c5CCC[C@@]65CCCc6sc(N)c(C#N)c65)nc43)cn2n1] 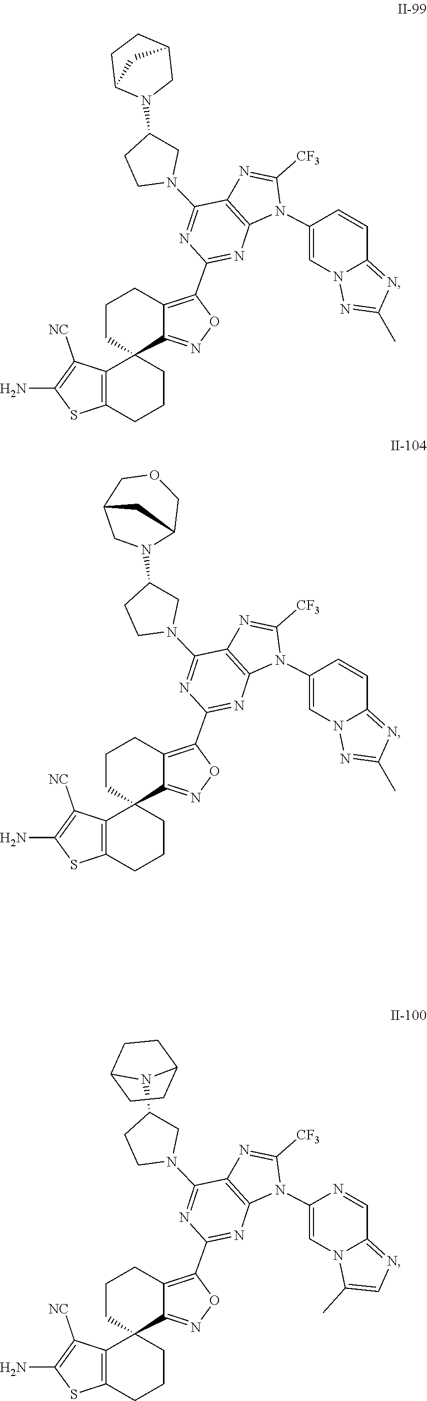 [SMILES: Cc1nc2ccc(-n3c(C(F)(F)F)nc4c(N5CC[C@H](N6C[C@@H]7CC[C@H]6C7)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N6C[C@@H]7COC[C@H]6C7)C5)nc(-c5onc6c5CCC[C@@]65CCCc6sc(N)c(C#N)c65)nc43)cn2n1 ; Cc1cnc2cnc(-n3c(C(F)(F)F)nc4c(N5CC[C@H](N6C7CCC6CC7)C5)nc(-c5onc6c5CCC[C@@]65CCCc6sc(N)c(C#N)c65)nc43)cn12] 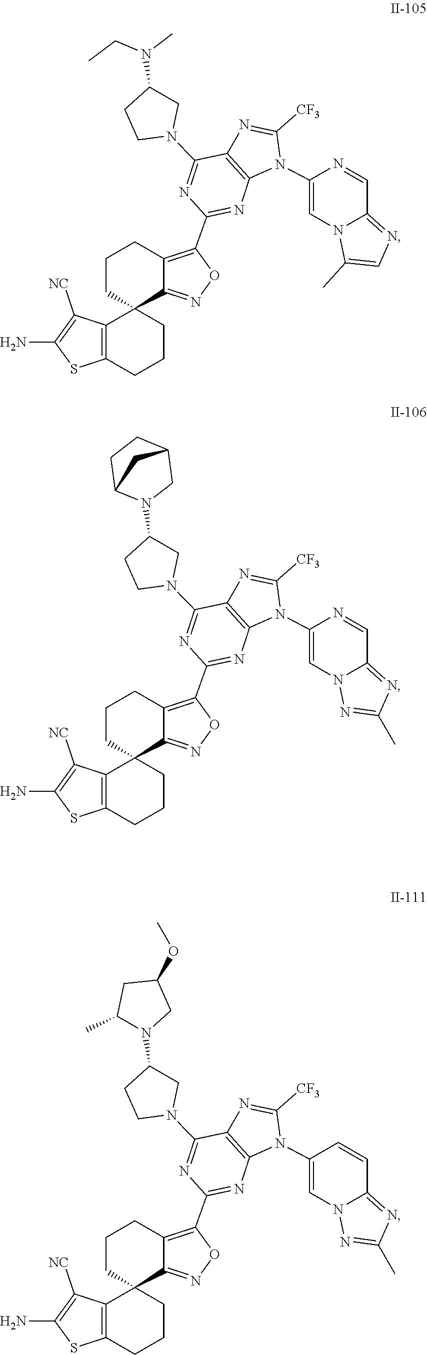 [SMILES: CCN(C)[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1 ; Cc1nc2cnc(-n3c(C(F)(F)F)nc4c(N5CC[C@H](N6C[C@H]7CC[C@@H]6C7)C5)nc(-c5onc6c5CCC[C@@]65CCCc6sc(N)c(C#N)c65)nc43)cn2n1 ; CO[C@@H]1C[C@@H](C)N([C@H]2CCN(c3nc(-c4onc5c4CCC[C@@]54CCCc5sc(N)c(C#N)c54)nc4c3nc(C(F)(F)F)n4-c3ccc4nc(C)nn4c3)C2)C1] 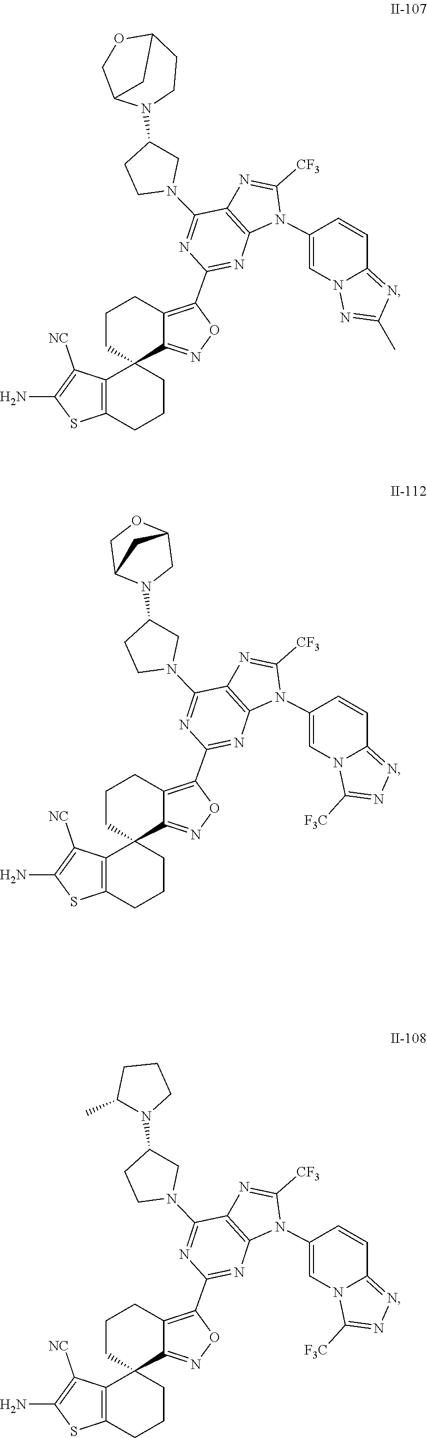 [SMILES: Cc1nc2ccc(-n3c(C(F)(F)F)nc4c(N5CC[C@H](N6CCC7CC6CO7)C5)nc(-c5onc6c5CCC[C@@]65CCCc6sc(N)c(C#N)c65)nc43)cn2n1 ; N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3ccc4nnc(C(F)(F)F)n4c3)c2n1 ; C[C@@H]1CCCN1[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1] 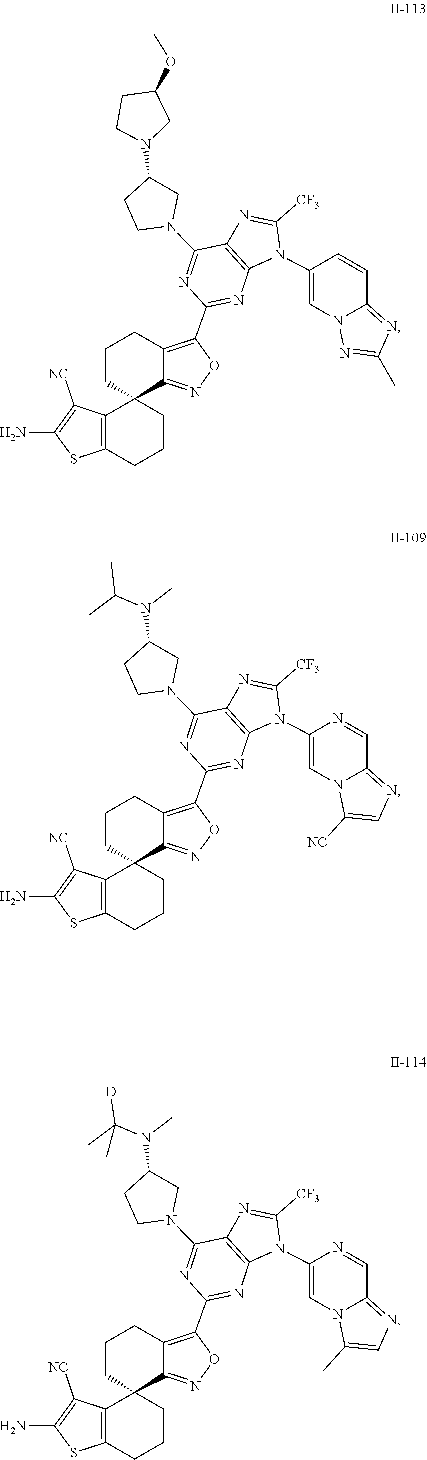 [SMILES: CO[C@@H]1CCN([C@H]2CCN(c3nc(-c4onc5c4CCC[C@@]54CCCc5sc(N)c(C#N)c54)nc4c3nc(C(F)(F)F)n4-c3ccc4nc(C)nn4c3)C2)C1 ; CC(C)N(C)[C@H]1CCN(c2nc(-c3onc4c3CCC[C@@]43CCCc4sc(N)c(C#N)c43)nc3c2nc(C(F)(F)F)n3-c2cn3c(C#N)cnc3cn2)C1 ; [2H]C(C)(C)N(C)[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] 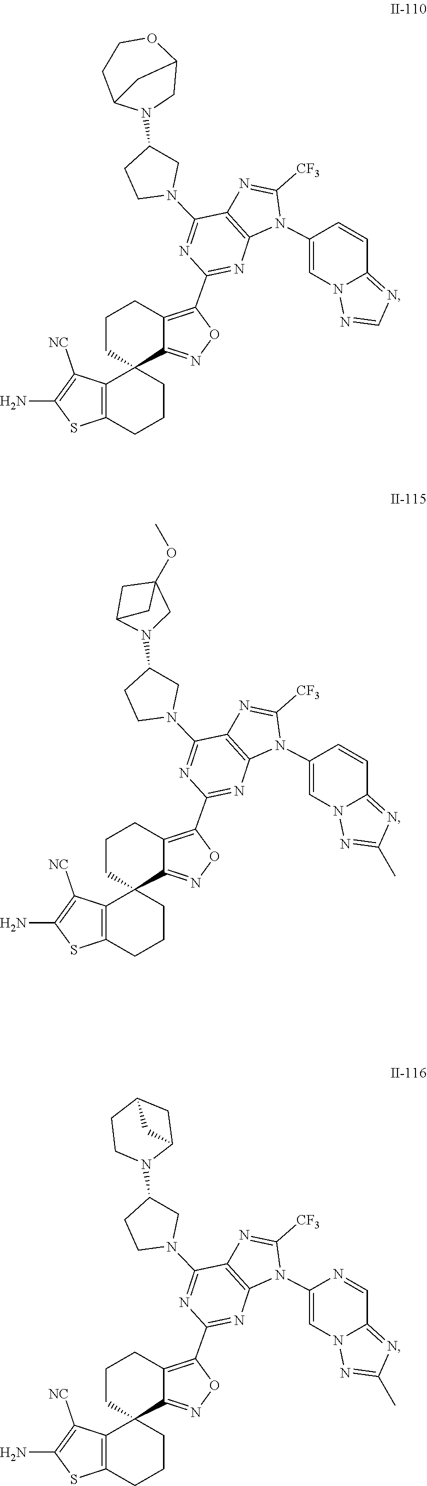 [SMILES: N#Cc1c(N)sc2c1[C@@]1(CCC2)CCCc2c1noc2-c1nc(N2CC[C@H](N3CC4CC3CCO4)C2)c2nc(C(F)(F)F)n(-c3ccc4ncnn4c3)c2n1 ; COC12CC(C1)N([C@H]1CCN(c3nc(-c4onc5c4CCC[C@@]54CCCc5sc(N)c(C#N)c54)nc4c3nc(C(F)(F)F)n4-c3ccc4nc(C)nn4c3)C1)C2 ; Cc1nc2cnc(-n3c(C(F)(F)F)nc4c(N5CC[C@H](N6CC[C@H]7C[C@@H]6C7)C5)nc(-c5onc6c5CCC[C@@]65CCCc6sc(N)c(C#N)c65)nc43)cn2n1] 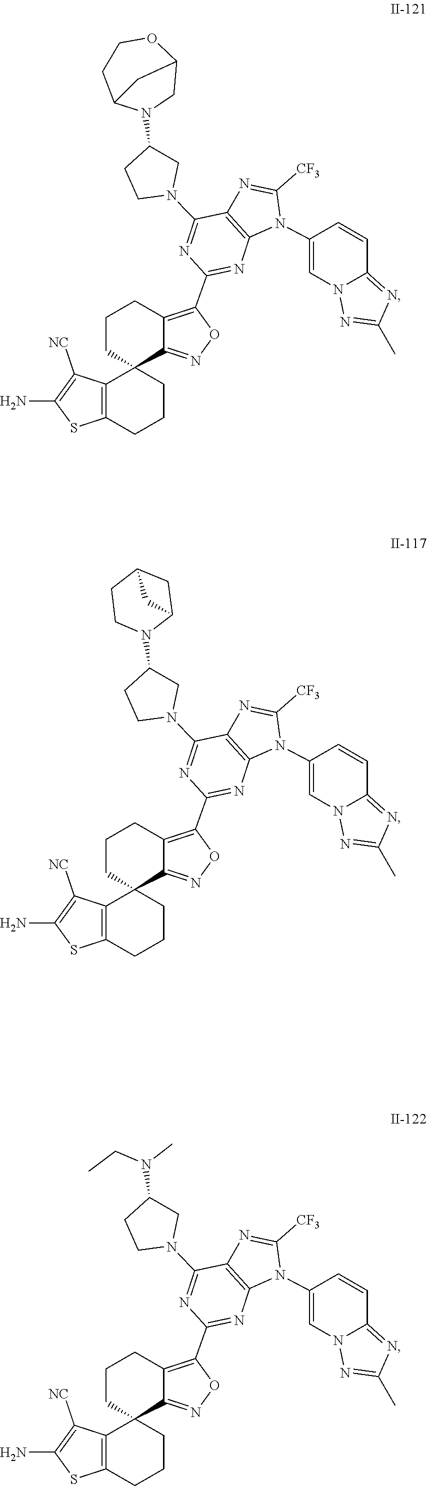 [SMILES: Cc1nc2ccc(-n3c(C(F)(F)F)nc4c(N5CC[C@H](N6CC7CC6CCO7)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N6CC[C@H]7C[C@@H]6C7)C5)nc(-c5onc6c5CCC[C@@]65CCCc6sc(N)c(C#N)c65)nc43)cn2n1 ; CCN(C)[C@H]1CCN(c2nc(-c3onc4c3CCC[C@@]43CCCc4sc(N)c(C#N)c43)nc3c2nc(C(F)(F)F)n3-c2ccc3nc(C)nn3c2)C1] 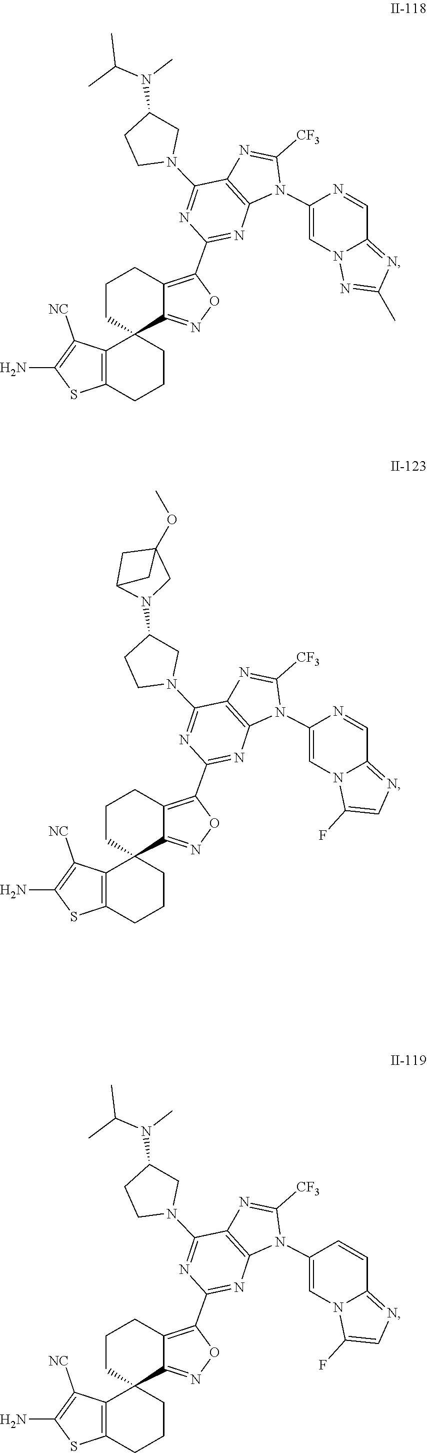 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2n1 ; COC12CC(C1)N([C@H]1CCN(c3nc(-c4onc5c4CCC[C@@]54CCCc5sc(N)c(C#N)c54)nc4c3nc(C(F)(F)F)n4-c3cn4c(F)cnc4cn3)C1)C2 ; CC(C)N(C)[C@H]1CCN(c2nc(-c3onc4c3CCC[C@@]43CCCc4sc(N)c(C#N)c43)nc3c2nc(C(F)(F)F)n3-c2ccc3ncc(F)n3c2)C1] 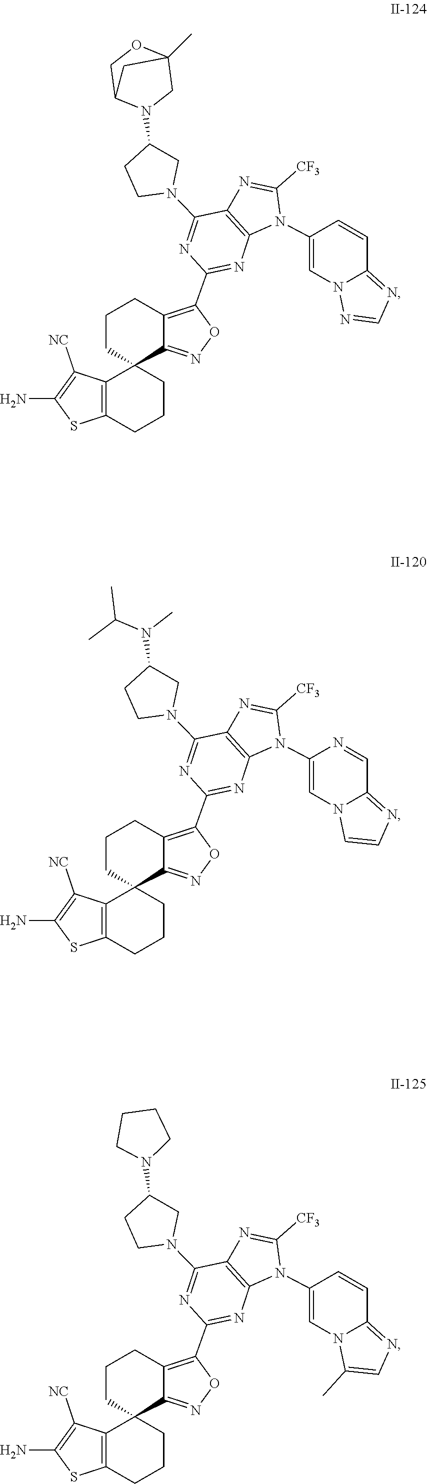 [SMILES: CC12CC(CO1)N([C@H]1CCN(c3nc(-c4onc5c4CCC[C@@]54CCCc5sc(N)c(C#N)c54)nc4c3nc(C(F)(F)F)n4-c3ccc4ncnn4c3)C1)C2 ; CC(C)N(C)[C@H]1CCN(c2nc(-c3onc4c3CCC[C@@]43CCCc4sc(N)c(C#N)c43)nc3c2nc(C(F)(F)F)n3-c2cn3ccnc3cn2)C1 ; Cc1cnc2ccc(-n3c(C(F)(F)F)nc4c(N5CC[C@H](N6CCCC6)C5)nc(-c5onc6c5CCC[C@@]65CCCc6sc(N)c(C#N)c65)nc43)cn12] 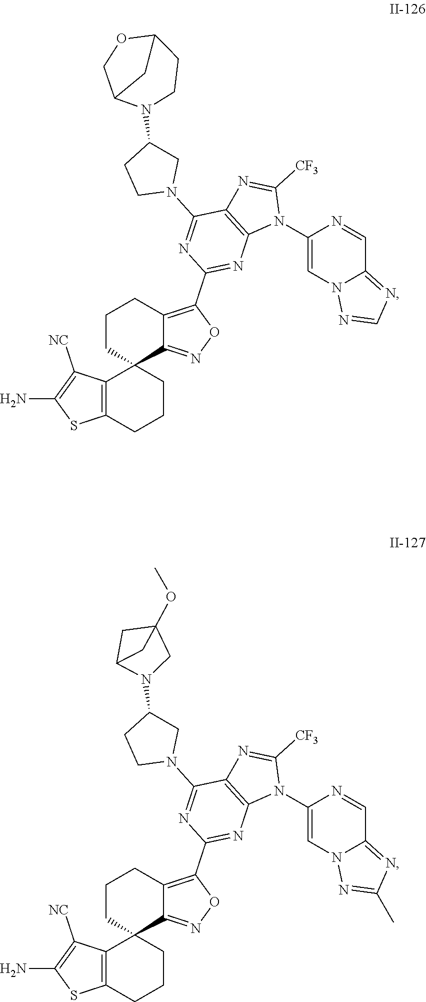 [SMILES: N#Cc1c(N)sc2c1[C@@]1(CCC2)CCCc2c1noc2-c1nc(N2CC[C@H](N3CCC4CC3CO4)C2)c2nc(C(F)(F)F)n(-c3cn4ncnc4cn3)c2n1 ; COC12CC(C1)N([C@H]1CCN(c3nc(-c4onc5c4CCC[C@@]54CCCc5sc(N)c(C#N)c54)nc4c3nc(C(F)(F)F)n4-c3cn4nc(C)nc4cn3)C1)C2] 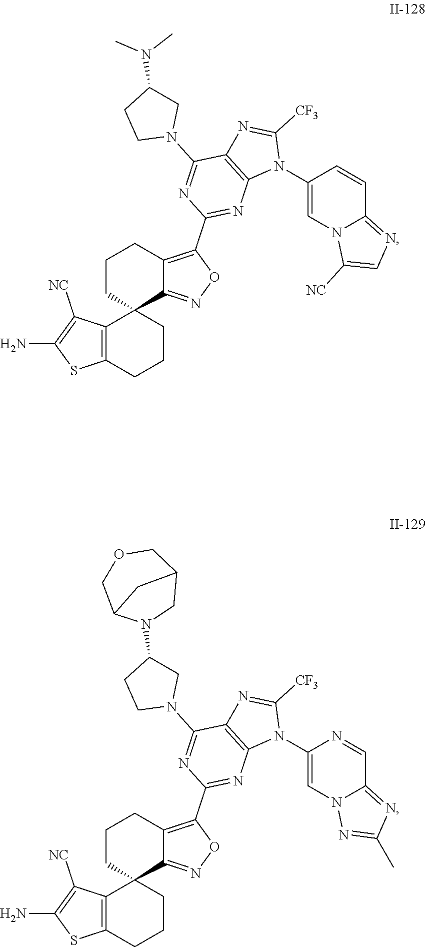 [SMILES: CN(C)[C@H]1CCN(c2nc(-c3onc4c3CCC[C@@]43CCCc4sc(N)c(C#N)c43)nc3c2nc(C(F)(F)F)n3-c2ccc3ncc(C#N)n3c2)C1 ; Cc1nc2cnc(-n3c(C(F)(F)F)nc4c(N5CC[C@H](N6CC7COCC6C7)C5)nc(-c5onc6c5CCC[C@@]65CCCc6sc(N)c(C#N)c65)nc43)cn2n1] 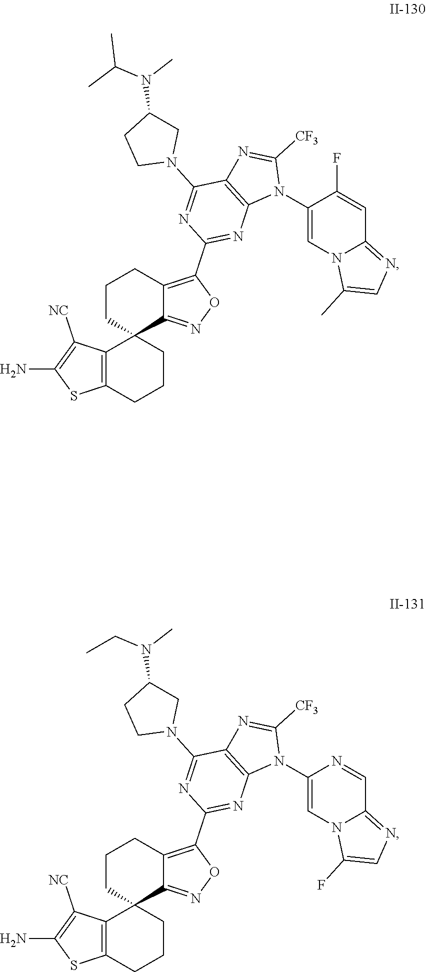 [SMILES: Cc1cnc2cc(F)c(-n3c(C(F)(F)F)nc4c(N5CC[C@H](N(C)C(C)C)C5)nc(-c5onc6c5CCC[C@@]65CCCc6sc(N)c(C#N)c65)nc43)cn12 ; CCN(C)[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1] 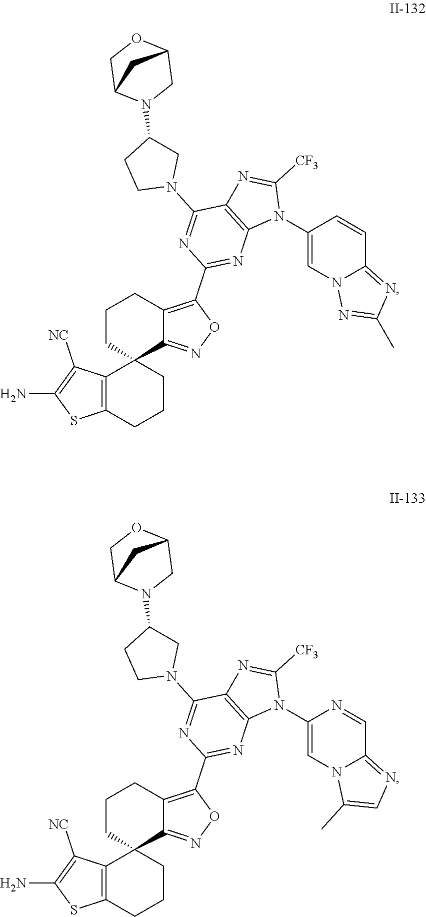 [SMILES: Cc1nc2ccc(-n3c(C(F)(F)F)nc4c(N5CC[C@H](N6C[C@@H]7C[C@H]6CO7)C5)nc(-c5onc6c5CCC[C@@]65CCCc6sc(N)c(C#N)c65)nc43)cn2n1 ; Cc1cnc2cnc(-n3c(C(F)(F)F)nc4c(N5CC[C@H](N6C[C@@H]7C[C@H]6CO7)C5)nc(-c5onc6c5CCC[C@@]65CCCc6sc(N)c(C#N)c65)nc43)cn12] 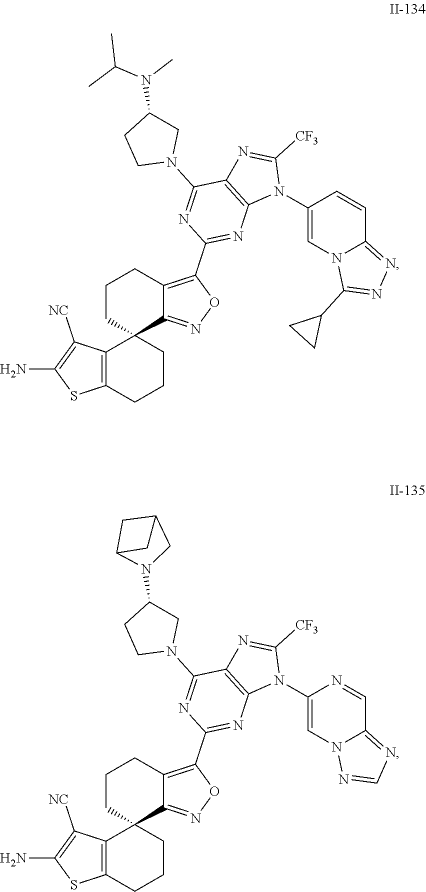 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3nnc(C4CC4)n3c2)C1 ; N#Cc1c(N)sc2c1[C@@]1(CCC2)CCCc2c1noc2-c1nc(N2CC[C@H](N3CC4CC3C4)C2)c2nc(C(F)(F)F)n(-c3cn4ncnc4cn3)c2n1] 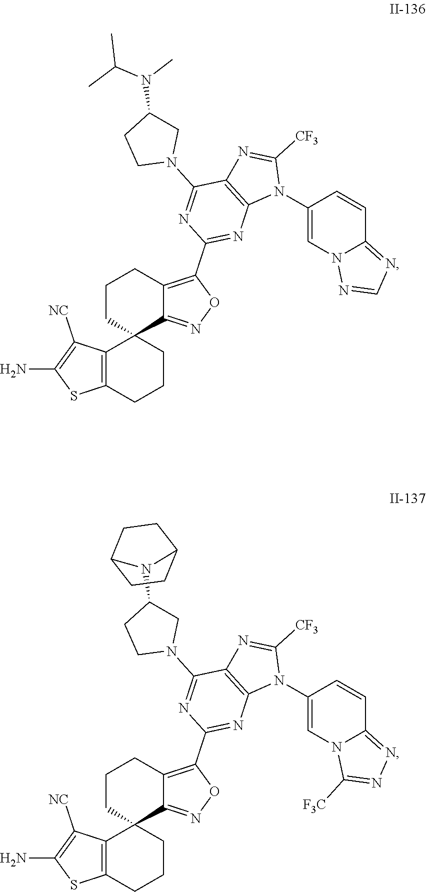 [SMILES: CC(C)N(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1 ; N#Cc1c(N)sc2c1[C@@]1(CCC2)CCCc2c1noc2-c1nc(N2CC[C@H](N3C4CCC3CC4)C2)c2nc(C(F)(F)F)n(-c3ccc4nnc(C(F)(F)F)n4c3)c2n1] 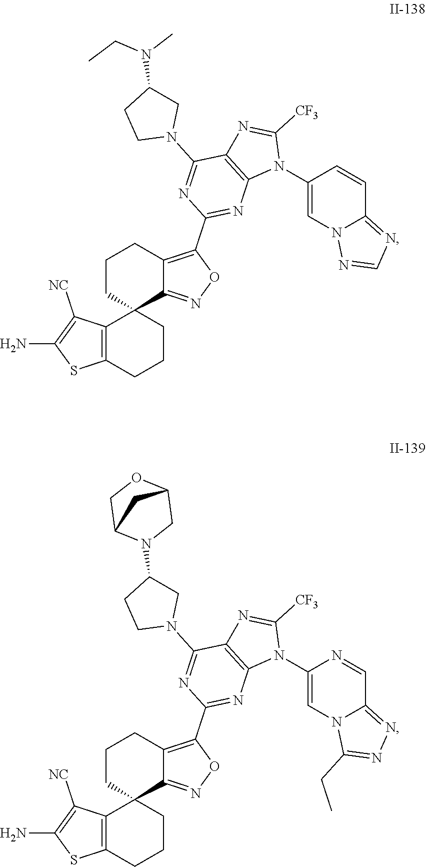 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3ncnn3c2)C1 ; CCc1nnc2cnc(-n3c(C(F)(F)F)nc4c(N5CC[C@H](N6C[C@@H]7C[C@H]6CO7)C5)nc(-c5onc6c5CCC[C@@]65CCCc6sc(N)c(C#N)c65)nc43)cn12] 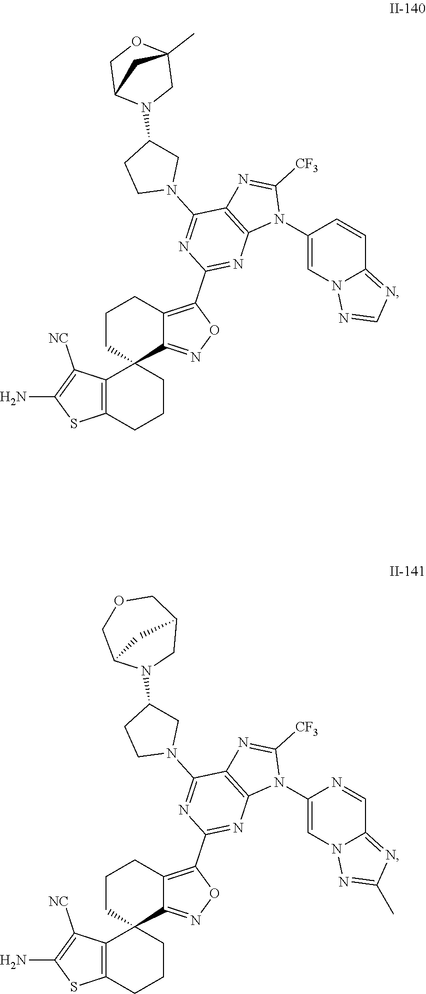 [SMILES: C[C@@]12C[C@@H](CO1)N([C@H]1CCN(c3nc(-c4onc5c4CCC[C@@]54CCCc5sc(N)c(C#N)c54)nc4c3nc(C(F)(F)F)n4-c3ccc4ncnn4c3)C1)C2 ; Cc1nc2cnc(-n3c(C(F)(F)F)nc4c(N5CC[C@H](N6C[C@@H]7COC[C@H]6C7)C5)nc(-c5onc6c5CCC[C@@]65CCCc6sc(N)c(C#N)c65)nc43)cn2n1] 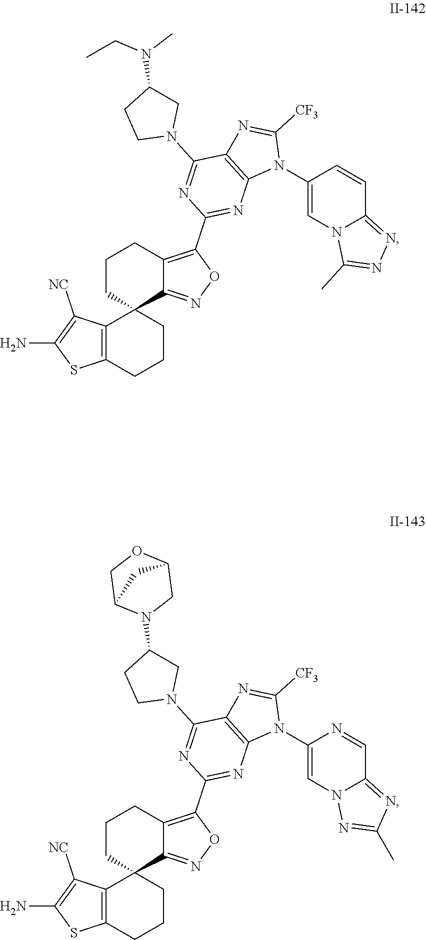 [SMILES: CCN(C)[C@H]1CCN(c2nc(-c3onc4c3CCC[C@@]43CCCc4sc(N)c(C#N)c43)nc3c2nc(C(F)(F)F)n3-c2ccc3nnc(C)n3c2)C1 ; Cc1nc2cnc(-n3c(C(F)(F)F)nc4c(N5CC[C@H](N6C[C@H]7C[C@@H]6CO7)C5)nc(-c5onc6c5CCC[C@@]65CCCc6sc(N)c(C#N)c65)nc43)cn2n1] 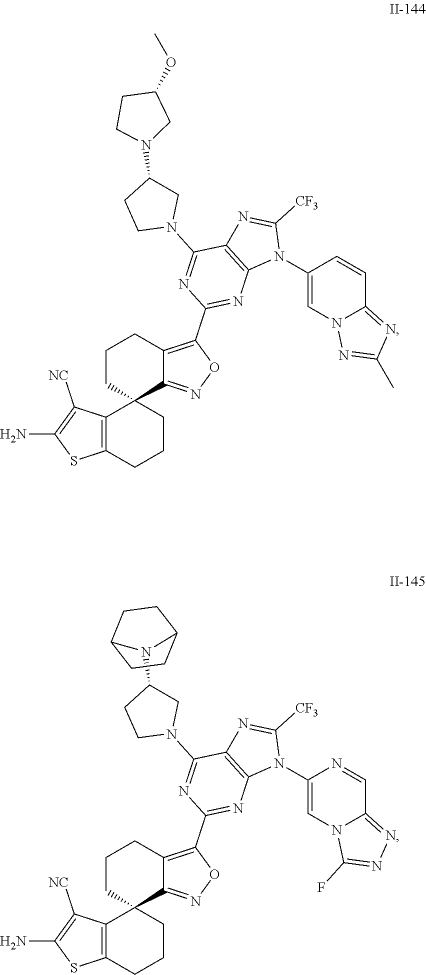 [SMILES: CO[C@H]1CCN([C@H]2CCN(c3nc(-c4onc5c4CCC[C@@]54CCCc5sc(N)c(C#N)c54)nc4c3nc(C(F)(F)F)n4-c3ccc4nc(C)nn4c3)C2)C1 ; N#Cc1c(N)sc2c1[C@@]1(CCC2)CCCc2c1noc2-c1nc(N2CC[C@H](N3C4CCC3CC4)C2)c2nc(C(F)(F)F)n(-c3cn4c(F)nnc4cn3)c2n1] 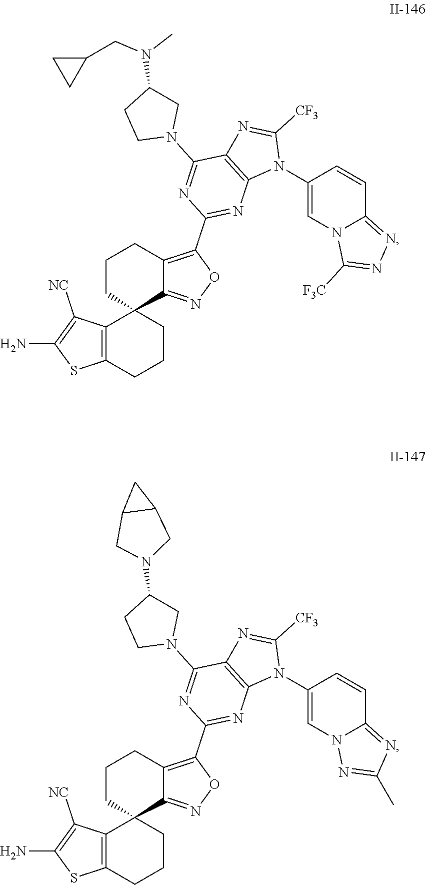 [SMILES: CN(CC1CC1)[C@H]1CCN(c2nc(-c3onc4c3CCC[C@@]43CCCc4sc(N)c(C#N)c43)nc3c2nc(C(F)(F)F)n3-c2ccc3nnc(C(F)(F)F)n3c2)C1 ; Cc1nc2ccc(-n3c(C(F)(F)F)nc4c(N5CC[C@H](N6CC7CC7C6)C5)nc(-c5onc6c5CCC[C@@]65CCCc6sc(N)c(C#N)c65)nc43)cn2n1] 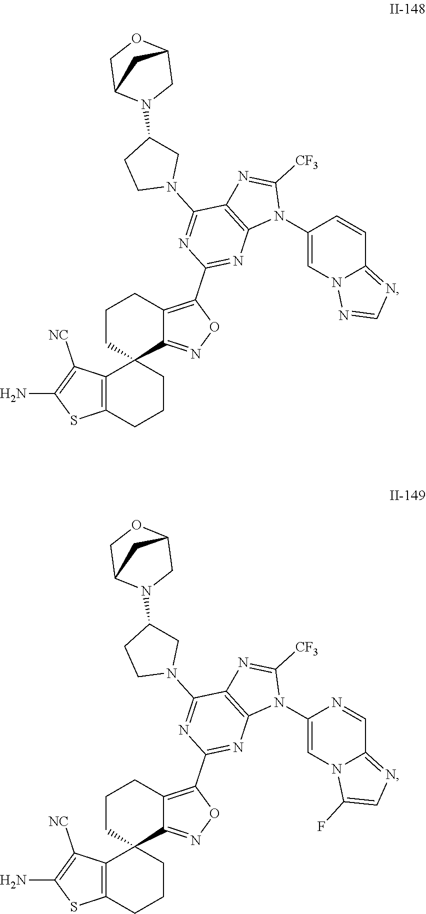 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3ccc4ncnn4c3)c2n1 ; N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3cn4c(F)cnc4cn3)c2n1] 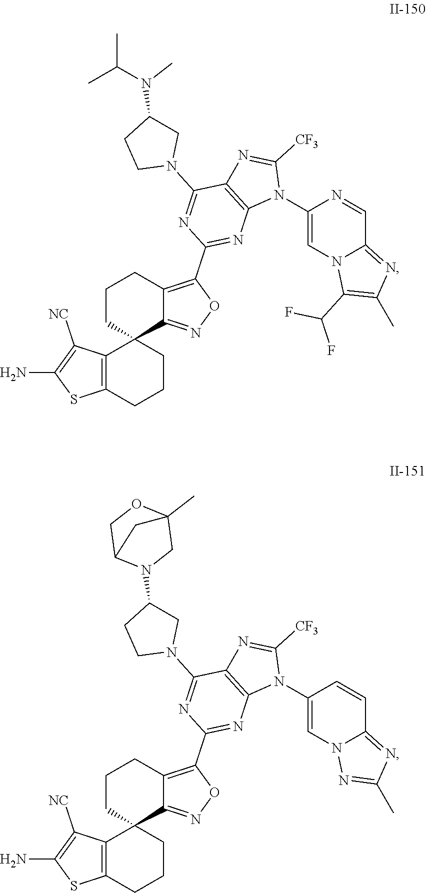 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cn2c1C(F)F ; Cc1nc2ccc(-n3c(C(F)(F)F)nc4c(N5CC[C@H](N6CC7(C)CC6CO7)C5)nc(-c5onc6c5CCC[C@@]65CCCc6sc(N)c(C#N)c65)nc43)cn2n1] 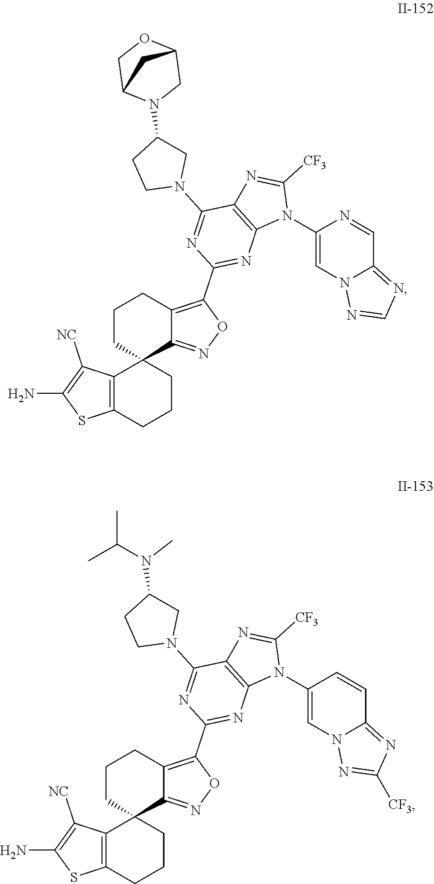 [SMILES: N#Cc1c(N)sc2c1[C@@]1(CCC2)CCCc2c1noc2-c1nc(N2CC[C@H](N3C[C@@H]4C[C@H]3CO4)C2)c2nc(C(F)(F)F)n(-c3cn4ncnc4cn3)c2n1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3nc(C(F)(F)F)nn3c2)C1] 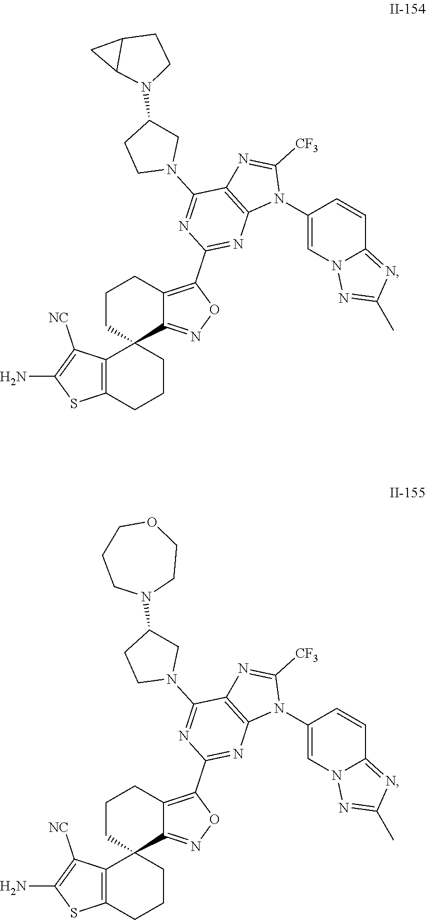 [SMILES: Cc1nc2ccc(-n3c(C(F)(F)F)nc4c(N5CC[C@H](N6CCC7CC76)C5)nc(-c5onc6c5CCC[C@@]65CCCc6sc(N)c(C#N)c65)nc43)cn2n1 ; Cc1nc2ccc(-n3c(C(F)(F)F)nc4c(N5CC[C@H](N6CCCOCC6)C5)nc(-c5onc6c5CCC[C@@]65CCCc6sc(N)c(C#N)c65)nc43)cn2n1] 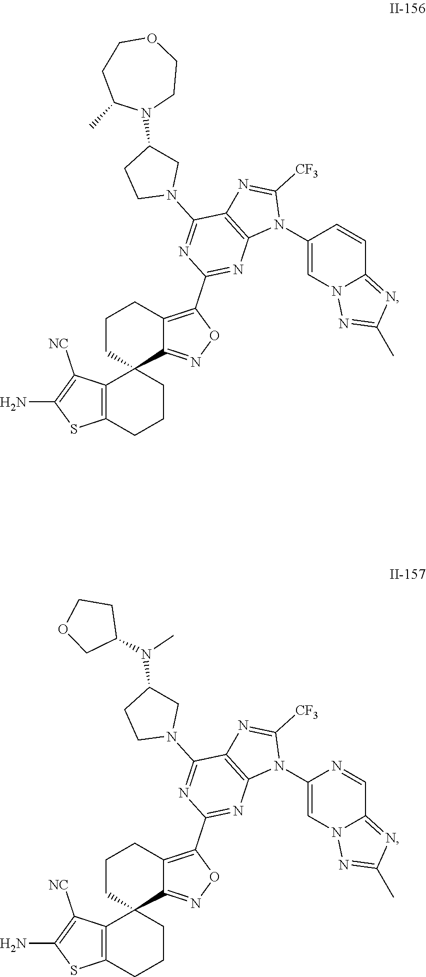 [SMILES: Cc1nc2ccc(-n3c(C(F)(F)F)nc4c(N5CC[C@H](N6CCOCC[C@H]6C)C5)nc(-c5onc6c5CCC[C@@]65CCCc6sc(N)c(C#N)c65)nc43)cn2n1 ; Cc1nc2cnc(-n3c(C(F)(F)F)nc4c(N5CC[C@H](N(C)[C@H]6CCOC6)C5)nc(-c5onc6c5CCC[C@@]65CCCc6sc(N)c(C#N)c65)nc43)cn2n1] 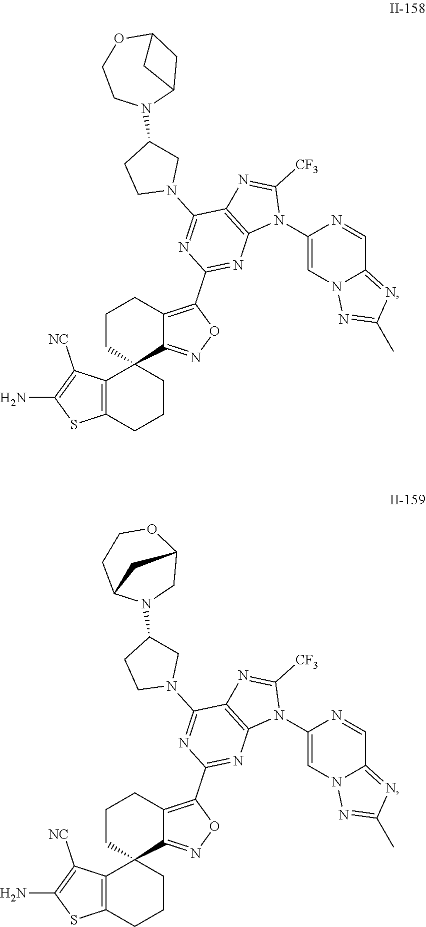 [SMILES: Cc1nc2cnc(-n3c(C(F)(F)F)nc4c(N5CC[C@H](N6CCOC7CC6C7)C5)nc(-c5onc6c5CCC[C@@]65CCCc6sc(N)c(C#N)c65)nc43)cn2n1 ; Cc1nc2cnc(-n3c(C(F)(F)F)nc4c(N5CC[C@H](N6C[C@@H]7C[C@H]6CCO7)C5)nc(-c5onc6c5CCC[C@@]65CCCc6sc(N)c(C#N)c65)nc43)cn2n1] 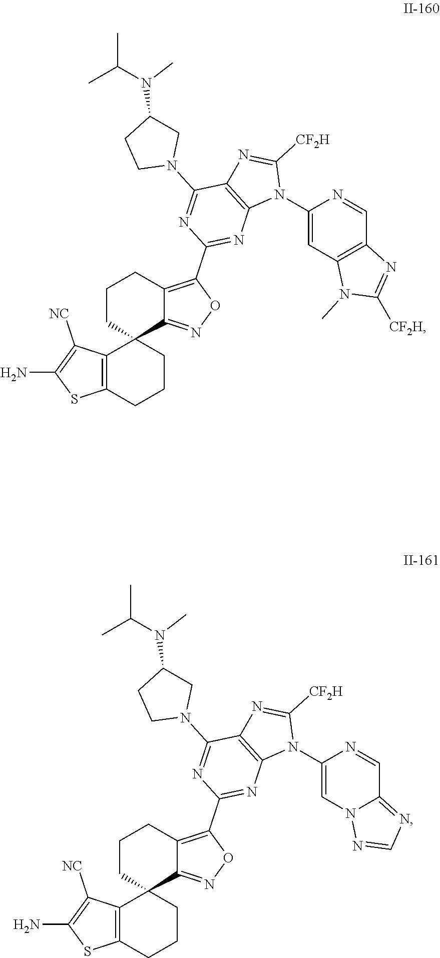 [SMILES: CC(C)N(C)[C@H]1CCN(c2nc(-c3onc4c3CCC[C@@]43CCCc4sc(N)c(C#N)c43)nc3c2nc(C(F)F)n3-c2cc3c(cn2)nc(C(F)F)n3C)C1 ; CC(C)N(C)[C@H]1CCN(c2nc(-c3onc4c3CCC[C@@]43CCCc4sc(N)c(C#N)c43)nc3c2nc(C(F)F)n3-c2cn3ncnc3cn2)C1] 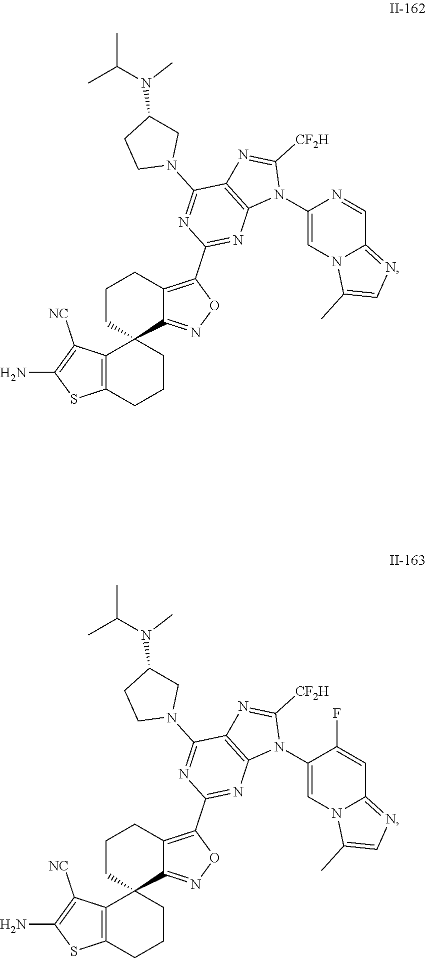 [SMILES: Cc1cnc2cnc(-n3c(C(F)F)nc4c(N5CC[C@H](N(C)C(C)C)C5)nc(-c5onc6c5CCC[C@@]65CCCc6sc(N)c(C#N)c65)nc43)cn12 ; Cc1cnc2cc(F)c(-n3c(C(F)F)nc4c(N5CC[C@H](N(C)C(C)C)C5)nc(-c5onc6c5CCC[C@@]65CCCc6sc(N)c(C#N)c65)nc43)cn12] 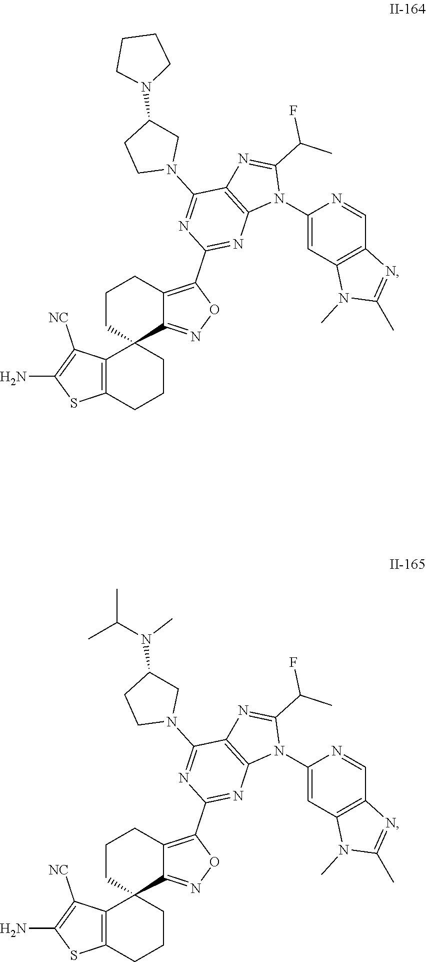 [SMILES: Cc1nc2cnc(-n3c(C(C)F)nc4c(N5CC[C@H](N6CCCC6)C5)nc(-c5onc6c5CCC[C@@]65CCCc6sc(N)c(C#N)c65)nc43)cc2n1C ; Cc1nc2cnc(-n3c(C(C)F)nc4c(N5CC[C@H](N(C)C(C)C)C5)nc(-c5onc6c5CCC[C@@]65CCCc6sc(N)c(C#N)c65)nc43)cc2n1C] 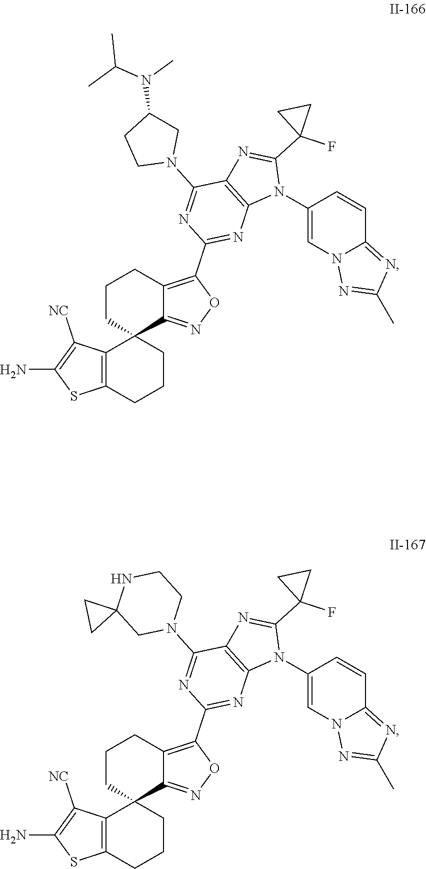 [SMILES: Cc1nc2ccc(-n3c(C4(F)CC4)nc4c(N5CC[C@H](N(C)C(C)C)C5)nc(-c5onc6c5CCC[C@@]65CCCc6sc(N)c(C#N)c65)nc43)cn2n1 ; Cc1nc2ccc(-n3c(C4(F)CC4)nc4c(N5CCNC6(CC6)C5)nc(-c5onc6c5CCC[C@@]65CCCc6sc(N)c(C#N)c65)nc43)cn2n1] 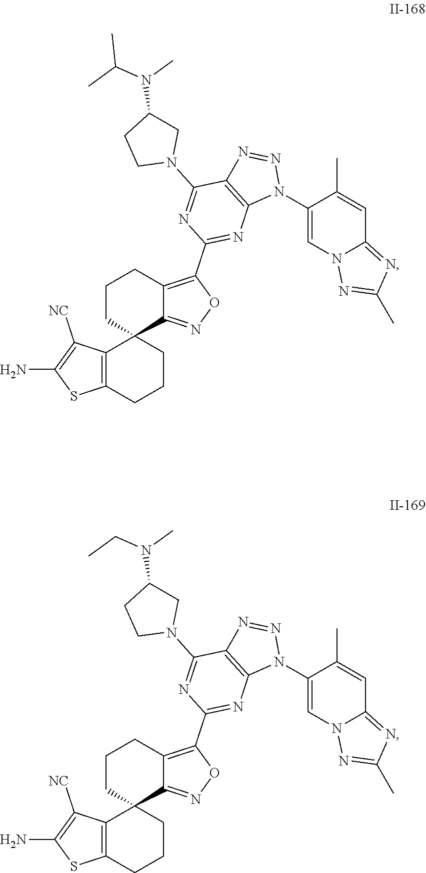 [SMILES: Cc1nc2cc(C)c(-n3nnc4c(N5CC[C@H](N(C)C(C)C)C5)nc(-c5onc6c5CCC[C@@]65CCCc6sc(N)c(C#N)c65)nc43)cn2n1 ; CCN(C)[C@H]1CCN(c2nc(-c3onc4c3CCC[C@@]43CCCc4sc(N)c(C#N)c43)nc3c2nnn3-c2cn3nc(C)nc3cc2C)C1] 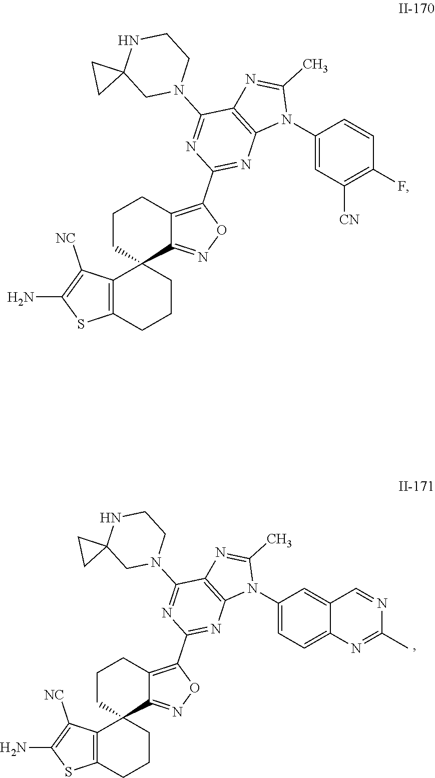 [SMILES: Cc1nc2c(N3CCNC4(CC4)C3)nc(-c3onc4c3CCC[C@@]43CCCc4sc(N)c(C#N)c43)nc2n1-c1ccc(F)c(C#N)c1 ; Cc1ncc2cc(-n3c(C)nc4c(N5CCNC6(CC6)C5)nc(-c5onc6c5CCC[C@@]65CCCc6sc(N)c(C#N)c65)nc43)ccc2n1] 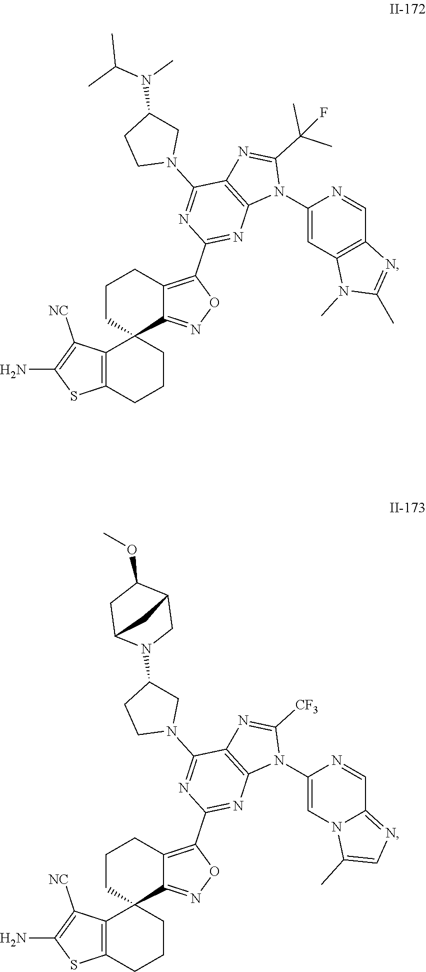 [SMILES: Cc1nc2cnc(-n3c(C(C)(C)F)nc4c(N5CC[C@H](N(C)C(C)C)C5)nc(-c5onc6c5CCC[C@@]65CCCc6sc(N)c(C#N)c65)nc43)cc2n1C ; CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] 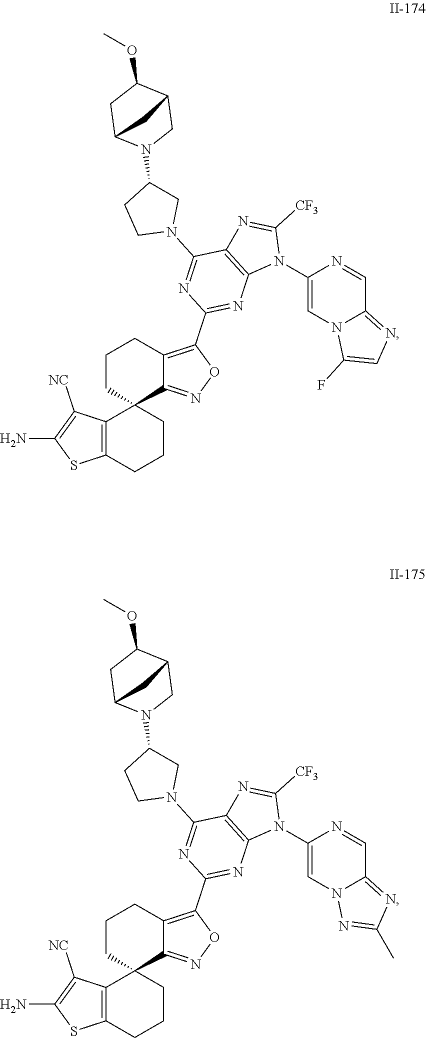 [SMILES: CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1 ; CO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3nc(C)nc3cn2)C1] 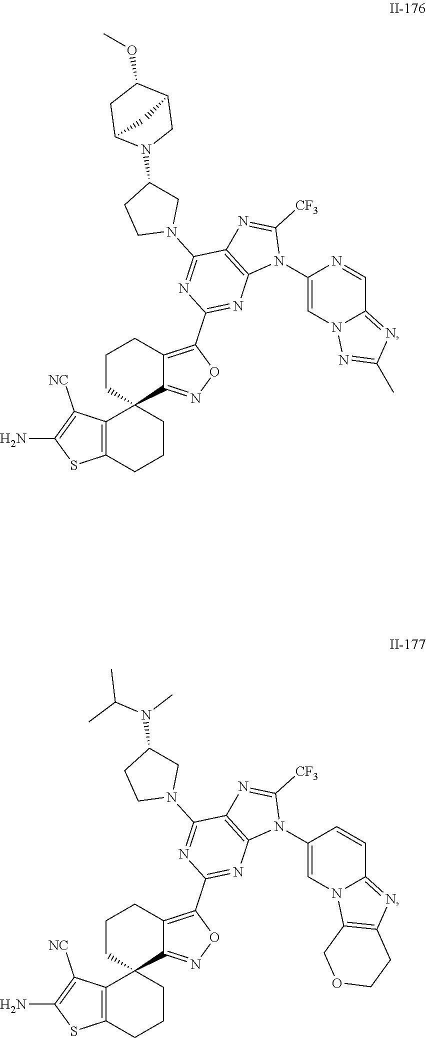 [SMILES: CO[C@H]1C[C@H]2C[C@@H]1CN2[C@H]1CCN(c2nc(-c3onc4c3CCC[C@@]43CCCc4sc(N)c(C#N)c43)nc3c2nc(C(F)(F)F)n3-c2cn3nc(C)nc3cn2)C1 ; CC(C)N(C)[C@H]1CCN(c2nc(-c3onc4c3CCC[C@@]43CCCc4sc(N)c(C#N)c43)nc3c2nc(C(F)(F)F)n3-c2ccc3nc4c(n3c2)COCC4)C1] 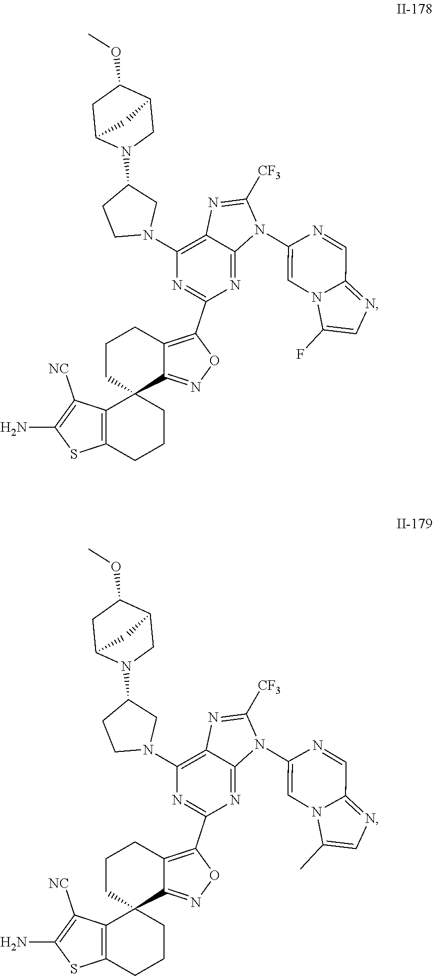 [SMILES: CO[C@H]1C[C@H]2C[C@@H]1CN2[C@H]1CCN(c2nc(-c3onc4c3CCC[C@@]43CCCc4sc(N)c(C#N)c43)nc3c2nc(C(F)(F)F)n3-c2cn3c(F)cnc3cn2)C1 ; CO[C@H]1C[C@H]2C[C@@H]1CN2[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] 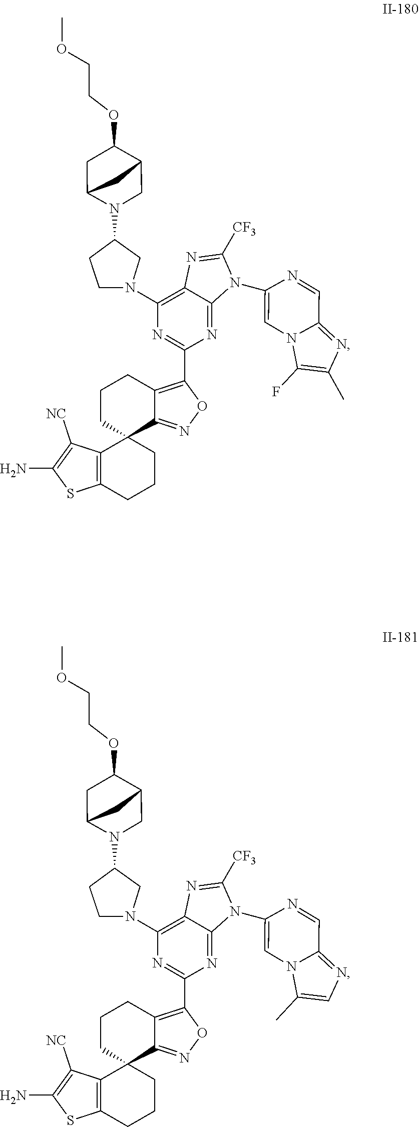 [SMILES: COCCO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(F)c(C)nc3cn2)C1 ; COCCO[C@@H]1C[C@@H]2C[C@H]1CN2[C@H]1CCN(c2nc(-c3onc4c3CCC[C@@]43CCCc4sc(N)c(C#N)c43)nc3c2nc(C(F)(F)F)n3-c2cn3c(C)cnc3cn2)C1] 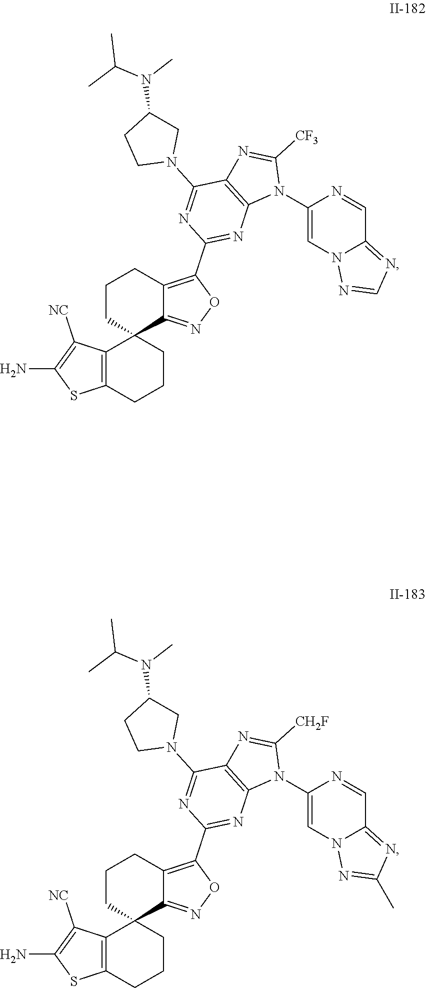 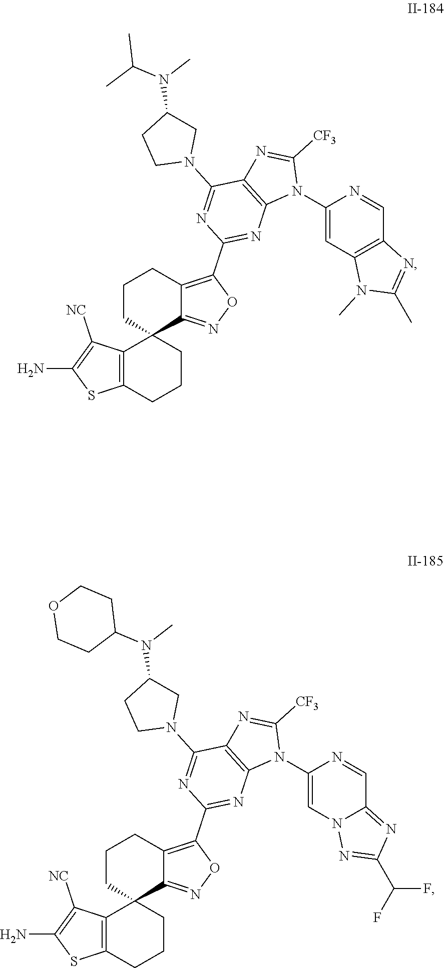 [SMILES: Cc1nc2cnc(-n3c(C(F)(F)F)nc4c(N5CC[C@H](N(C)C(C)C)C5)nc(-c5onc6c5CCC[C@@]65CCCc6sc(N)c(C#N)c65)nc43)cc2n1C ; CN(C1CCOCC1)[C@H]1CCN(c2nc(-c3onc4c3CCC[C@@]43CCCc4sc(N)c(C#N)c43)nc3c2nc(C(F)(F)F)n3-c2cn3nc(C(F)F)nc3cn2)C1] 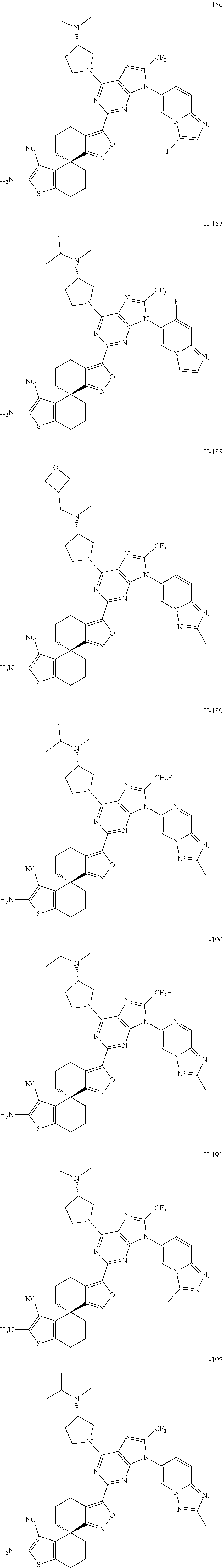 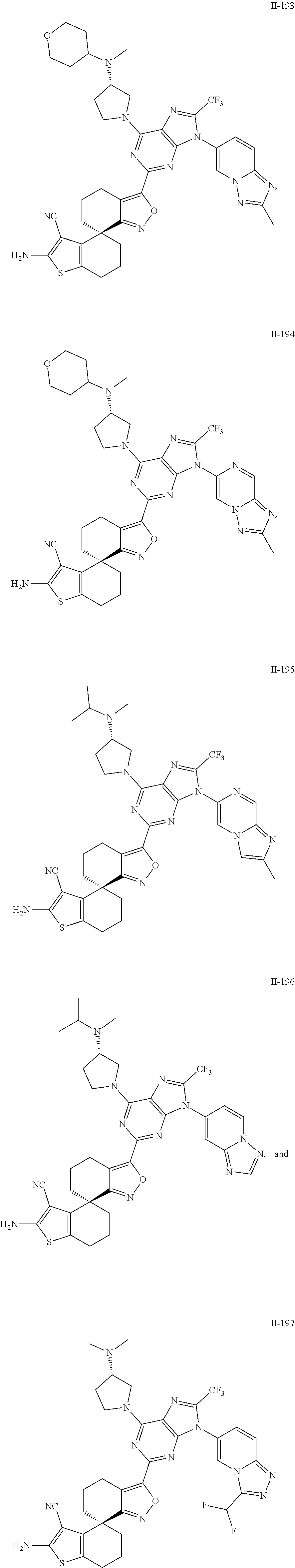
or a salt thereof.

13. A method of treating or preventing a disease or condition mediated by KRAS comprising administering a therapeutically effective amount of a compound according to claim 1—or a pharmaceutically acceptable salt.

14. A method of treating or preventing cancer comprising administering a therapeutically effective amount of a compound according to claim 1 or a pharmaceutically acceptable salt thereof.

15. The method of claim 14, wherein said compound or salt is administered in combination with one or more other pharmacologically active substance(s).

16. The method of claim 14, wherein the cancer is selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, gastroesophageal cancer, chronic lymphocytic leukemia, hepatocellular cancer, breast cancer, ovarian cancer, prostate cancer, glioblastoma, renal cancer and sarcoma.

17. The method of claim 14, wherein the cancer comprises tumor cells harboring a KRAS G12D mutation.

18. A pharmaceutical composition comprising a compound according to claim 1 or a pharmaceutically acceptable salt thereof and one or more other pharmaceutically acceptable excipient(s).
